SEQUENCE LISTING (548 sequences)

SEQ ID NO: 1 - DNA, 44 bp, Artificial Sequence
  Description of Artificial Sequence Synthetic probe
actggtaacccagacatgatcggttataactaccaccacgctga

SEQ ID NO: 2 - DNA, 44 bp, Artificial Sequence
  Description of Artificial Sequence Synthetic probe
actccctgtgggtgagcttaatggtataactaccaccacgctgg

SEQ ID NO: 3 - DNA, 51 bp, Artificial Sequence
  Description of Artificial Sequence Synthetic probe
catttggtcattggttcaagacgagttcctggcaattaattgtaaaaggtg

SEQ ID NO: 4 - DNA, 51 bp, Artificial Sequence
  Description of Artificial Sequence Synthetic probe
ctggtccgttgtggtccttctaacgttcctggcaattaattgtaaaaggta

SEQ ID NO: 5 - DNA, 26 bp, Artificial Sequence
  Description of Artificial Sequence Synthetic probe
ctaaaccatgtgtcaaaatcagcatg

SEQ ID NO: 6 - DNA, 28 bp, Artificial Sequence
  Description of Artificial Sequence Synthetic probe
aacaggaaactgtataattaccgatatc

SEQ ID NO: 7 - DNA, 20 bp, Artificial Sequence
  Description of Artificial Sequence Synthetic oligonucleotide
tataactaccaccacgctgr

SEQ ID NO: 8 - DNA, 26 bp, Artificial Sequence
  Description of Artificial Sequence Synthetic oligonucleotide
ctaaaccatgtgtcaaaatcagcatg

SEQ ID NO: 9 - DNA, 27 bp, Artificial Sequence
  Description of Artificial Sequence Synthetic oligonucleotide
gttcctggcaattaattgtaaaaggtr

SEQ ID NO: 10 - DNA, 28 bp, Artificial Sequence
  Description of Artificial Sequence Synthetic oligonucleotide
aacaggaaactgtataattaccgatatc

SEQ ID NO: 11 - DNA, 21 bp, Artificial Sequence
  Description of Artificial Sequence Synthetic primer
aggctattgatggtggtgtca

SEQ ID NO: 12 - DNA, 20 bp, Artificial Sequence
  Description of Artificial Sequence Synthetic primer
gcacgacaaaacccacttct

SEQ ID NO: 13 - DNA, 20 bp, Artificial Sequence
  Description of Artificial Sequence Synthetic primer
cgtggatgaggctggttcta

SEQ ID NO: 14 - DNA, 20 bp, Artificial Sequence
  Description of Artificial Sequence Synthetic primer
tttcgctgattttggggtcc

SEQ ID NO: 15 - DNA, 46 bp, Artificial Sequence
  Description of Artificial Sequence Synthetic probe
ctaatagctcctgtgccctcgtatgggacttctgtgcagttaacat

SEQ ID NO: 16 - DNA, 46 bp, Artificial Sequence
  Description of Artificial Sequence Synthetic probe
aatccgtcgactagcctgagaattgggacttctgtgcagttaacac

SEQ ID NO: 17 - DNA, 23 bp, Artificial Sequence
  Description of Artificial Sequence Synthetic probe
cctgataaagaacagcaacctgg

SEQ ID NO: 18 - DNA, 22 bp, Artificial Sequence
  Description of Artificial Sequence Synthetic oligonucleotide
gggacttctgtgcagttaacay

SEQ ID NO: 19 - DNA, 23 bp, Artificial Sequence
  Description of Artificial Sequence Synthetic oligonucleotide
cctgataaagaacagcaacctgg

SEQ ID NO: 20 - DNA, 20 bp, Artificial Sequence
  Description of Artificial Sequence Synthetic primer
acactactgatgctgtccgt

SEQ ID NO: 21 - DNA, 20 bp, Artificial Sequence
  Description of Artificial Sequence Synthetic primer
gcccctattaaacagcctgc

SEQ ID NO: 22 - DNA, 44 bp, Artificial Sequence
  Description of Artificial Sequence Synthetic probe
gtggcaacagaaatcaggtggtgacgaaatgcaccccgcattac

SEQ ID NO: 23 - DNA, 21 bp, Artificial Sequence
  Description of Artificial Sequence Synthetic probe
gtttggtggaccctcagattc

SEQ ID NO: 24 - DNA, 45 bp, Artificial Sequence
  Description of Artificial Sequence Synthetic probe
gtggcaacagaaatcaggtggtgagaatctgagggtccaccaaac

SEQ ID NO: 25 - DNA, 45 bp, Artificial Sequence
  Description of Artificial Sequence Synthetic probe
tagatgccgctgcaggtatggaaagaatctgagggtccaccaaac

SEQ ID NO: 26 - DNA, 20 bp, Artificial Sequence
  Description of Artificial Sequence Synthetic probe
gtaatgcggggtgcatttcg

SEQ ID NO: 27 - DNA, 45 bp, Artificial Sequence
  Description of Artificial Sequence Synthetic probe
tagatgccgctgcaggtatggaaagcaaattgcacaatttgcccc

SEQ ID NO: 28 - DNA, 18 bp, Artificial Sequence
  Description of Artificial Sequence Synthetic probe
cagcgcttcagcgttctt

SEQ ID NO: 29 - DNA, 41 bp, Artificial Sequence
  Description of Artificial Sequence Synthetic probe
tagatgccgctgcaggtatggaaagaacgctgaagcgctgg

SEQ ID NO: 30 - DNA, 21 bp, Artificial Sequence
  Description of Artificial Sequence Synthetic probe
gggcaaattgtgcaatttgcg

SEQ ID NO: 31 - DNA, 45 bp, Artificial Sequence
  Description of Artificial Sequence Synthetic probe
agaggactgctaaaggtttgtaggccaaaagaycacattggcacc

SEQ ID NO: 32 - DNA, 22 bp, Artificial Sequence
  Description of Artificial Sequence Synthetic probe
cgcaatcctgctaacaatgctg

SEQ ID NO: 33 - DNA, 45 bp, Artificial Sequence
  Description of Artificial Sequence Synthetic probe
agaggactgctaaaggtttgtagggcattgttagcaggattgcgg

SEQ ID NO: 34 - DNA, 22 bp, Artificial Sequence
  Description of Artificial Sequence Synthetic probe
gtgccaatgtgrtcttttggtg

SEQ ID NO: 35 - DNA, 42 bp, Artificial Sequence
  Description of Artificial Sequence Synthetic probe
cgtaccattgaatctggagaccttgagcgggttctgacctga

SEQ ID NO: 36 - DNA, 16 bp, Artificial Sequence
  Description of Artificial Sequence Synthetic probe
aggctctgcgcggact

SEQ ID NO: 37 - DNA, 41 bp, Artificial Sequence
  Description of Artificial Sequence Synthetic probe
cgtaccattgaatctggagaccttcaagtccgcgcagagcc

SEQ ID NO: 38 - DNA, 18 bp, Artificial Sequence
  Description of Artificial Sequence Synthetic probe
ttcaggtcagaacccgct

SEQ ID NO: 39 - DNA, 20 bp, Artificial Sequence
  Description of Artificial Sequence Synthetic oligonucleotide
cgaaatgcaccccgcattac

SEQ ID NO: 40 - DNA, 21 bp, Artificial Sequence
  Description of Artificial Sequence Synthetic oligonucleotide
gtttggtggaccctcagattc

SEQ ID NO: 41 - DNA, 21 bp, Artificial Sequence
  Description of Artificial Sequence Synthetic oligonucleotide
gaatctgagggtccaccaaac

SEQ ID NO: 42 - DNA, 20 bp, Artificial Sequence
  Description of Artificial Sequence Synthetic oligonucleotide
gtaatgcggggtgcatttcg

SEQ ID NO: 43 - DNA, 21 bp, Artificial Sequence
  Description of Artificial Sequence Synthetic oligonucleotide
gcaaattgcacaatttgcccc

SEQ ID NO: 44 - DNA, 18 bp, Artificial Sequence
  Description of Artificial Sequence Synthetic oligonucleotide
cagcgcttcagcgttctt

SEQ ID NO: 45 - DNA, 17 bp, Artificial Sequence
  Description of Artificial Sequence Synthetic oligonucleotide
gaacgctgaagcgctgg

SEQ ID NO: 46 - DNA, 21 bp, Artificial Sequence
  Description of Artificial Sequence Synthetic oligonucleotide
gggcaaattgtgcaatttgcg

SEQ ID NO: 47 - DNA, 21 bp, Artificial Sequence
  Description of Artificial Sequence Synthetic oligonucleotide
ccaaaagaycacattggcacc

SEQ ID NO: 48 - DNA, 22 bp, Artificial Sequence
  Description of Artificial Sequence Synthetic oligonucleotide
cgcaatcctgctaacaatgctg

SEQ ID NO: 49 - DNA, 21 bp, Artificial Sequence
  Description of Artificial Sequence Synthetic oligonucleotide
gcattgttagcaggattgcgg

SEQ ID NO: 50 - DNA, 22 bp, Artificial Sequence
  Description of Artificial Sequence Synthetic oligonucleotide
gtgccaatgtgrtcttttggtg

SEQ ID NO: 51 - DNA, 18 bp, Artificial Sequence
  Description of Artificial Sequence Synthetic oligonucleotide
gagcgggttctgacctga

SEQ ID NO: 52 - DNA, 16 bp, Artificial Sequence
  Description of Artificial Sequence Synthetic oligonucleotide
aggctctgcgcggact

SEQ ID NO: 53 - DNA, 17 bp, Artificial Sequence
  Description of Artificial Sequence Synthetic oligonucleotide
caagtccgcgcagagcc

SEQ ID NO: 54 - DNA, 18 bp, Artificial Sequence
  Description of Artificial Sequence Synthetic oligonucleotide
ttcaggtcagaacccgct

SEQ ID NO: 55 - DNA, 44 bp, Artificial Sequence
  Description of Artificial Sequence Synthetic primer
aatccgtcgactagcctgagaattgaccccaaaatcagcgaaat

SEQ ID NO: 56 - DNA, 24 bp, Artificial Sequence
  Description of Artificial Sequence Synthetic primer
tctggttactgccagttgaatctg

SEQ ID NO: 57 - DNA, 24 bp, Artificial Sequence
  Description of Artificial Sequence Synthetic probe
accccgcattacgtttggtggacc

SEQ ID NO: 58 - DNA, 44 bp, Artificial Sequence
  Description of Artificial Sequence Synthetic primer
agaggactgctaaaggtttgtaggttacaaacattggccgcaaa

SEQ ID NO: 59 - DNA, 18 bp, Artificial Sequence
  Description of Artificial Sequence Synthetic primer
gcgcgacattccgaagaa

SEQ ID NO: 60 - DNA, 23 bp, Artificial Sequence
  Description of Artificial Sequence Synthetic probe
acaatttgcccccagcgcttcag

SEQ ID NO: 61 - DNA, 46 bp, Artificial Sequence
  Description of Artificial Sequence Synthetic primer
cgtaccattgaatctggagaccttgggagccttgaatacaccaaaa

SEQ ID NO: 62 - DNA, 21 bp, Artificial Sequence
  Description of Artificial Sequence Synthetic primer
tgtagcacgattgcagcattg

SEQ ID NO: 63 - DNA, 24 bp, Artificial Sequence
  Description of Artificial Sequence Synthetic probe
aycacattggcacccgcaatcctg

SEQ ID NO: 64 - DNA, 43 bp, Artificial Sequence
  Description of Artificial Sequence Synthetic primer
actggtaacccagacatgatcggtagatttggacctgcgagcg

SEQ ID NO: 65 - DNA, 20 bp, Artificial Sequence
  Description of Artificial Sequence Synthetic primer
gagcggctgtctccacaagt

SEQ ID NO: 66 - DNA, 23 bp, Artificial Sequence
  Description of Artificial Sequence Synthetic probe
ttctgacctgaaggctctgcgcg

SEQ ID NO: 67 - DNA, 20 bp, Artificial Sequence
  Description of Artificial Sequence Synthetic primer
gaccccaaaatcagcgaaat

SEQ ID NO: 68 - DNA, 24 bp, Artificial Sequence
  Description of Artificial Sequence Synthetic primer
tctggttactgccagttgaatctg

SEQ ID NO: 69 - DNA, 20 bp, Artificial Sequence
  Description of Artificial Sequence Synthetic primer
ttacaaacattggccgcaaa

SEQ ID NO: 70 - DNA, 18 bp, Artificial Sequence
  Description of Artificial Sequence Synthetic primer
gcgcgacattccgaagaa

SEQ ID NO: 71 - DNA, 22 bp, Artificial Sequence
  Description of Artificial Sequence Synthetic primer
gggagccttgaatacaccaaaa

SEQ ID NO: 72 - DNA, 21 bp, Artificial Sequence
  Description of Artificial Sequence Synthetic primer
tgtagcacgattgcagcattg

SEQ ID NO: 73 - DNA, 19 bp, Artificial Sequence
  Description of Artificial Sequence Synthetic primer
agatttggacctgcgagcg

SEQ ID NO: 74 - DNA, 20 bp, Artificial Sequence
  Description of Artificial Sequence Synthetic primer
gagcggctgtctccacaagt

SEQ ID NO: 75 - DNA, 49 bp, Artificial Sequence
  Description of Artificial Sequence Synthetic probe
actccctgtgggtgagcttaatgggttacttggttccatgctatacatg

SEQ ID NO: 76 - DNA, 48 bp, Artificial Sequence
  Description of Artificial Sequence Synthetic probe
actggtaacccagacatgatcggtccaatgttacttggttccatgcta

SEQ ID NO: 77 - DNA, 23 bp, Artificial Sequence
  Description of Artificial Sequence Synthetic probe
tctctgggaccaatggtactaag

SEQ ID NO: 78 - DNA, 46 bp, Artificial Sequence
  Description of Artificial Sequence Synthetic probe
actccctgtgggtgagcttaatggccattggtcccagagacatgta

SEQ ID NO: 79 - DNA, 47 bp, Artificial Sequence
  Description of Artificial Sequence Synthetic probe
actggtaacccagacatgatcggtcttagtaccattggtcccagaga

SEQ ID NO: 80 - DNA, 24 bp, Artificial Sequence
  Description of Artificial Sequence Synthetic probe
tagcatggaaccaagtaacattgg

SEQ ID NO: 81 - DNA, 48 bp, Artificial Sequence
  Description of Artificial Sequence Synthetic probe
catttggtcattggttcaagacgaaatcatatggtttccaacccacta

SEQ ID NO: 82 - DNA, 48 bp, Artificial Sequence
  Description of Artificial Sequence Synthetic probe
ctggtccgttgtggtccttctaacaatcatatggtttccaacccactt

SEQ ID NO: 83 - DNA, 23 bp, Artificial Sequence
  Description of Artificial Sequence Synthetic probe
atggtgttggttaccaaccatac

SEQ ID NO: 84 - DNA, 47 bp, Artificial Sequence
  Description of Artificial Sequence Synthetic probe
catttggtcattggttcaagacgatatggttggtaaccaacaccatt

SEQ ID NO: 85 - DNA, 47 bp, Artificial Sequence
  Description of Artificial Sequence Synthetic probe
ctggtccgttgtggtccttctaactatggttggtaaccaacaccata

SEQ ID NO: 86 - DNA, 24 bp, Artificial Sequence
  Description of Artificial Sequence Synthetic probe
agtgggttggaaaccatatgattg

SEQ ID NO: 87 - DNA, 52 bp, Artificial Sequence
  Description of Artificial Sequence Synthetic probe
gtggcaacagaaatcaggtggtgactagttatcagactcagactaattctcc

SEQ ID NO: 88 - DNA, 52 bp, Artificial Sequence
  Description of Artificial Sequence Synthetic probe
agaggactgctaaaggtttgtaggctagttatcagactcagactaattctca

SEQ ID NO: 89 - DNA, 15 bp, Artificial Sequence
  Description of Artificial Sequence Synthetic probe
tcggcgggcacgtag

SEQ ID NO: 90 - DNA, 40 bp, Artificial Sequence
  Description of Artificial Sequence Synthetic probe
gtggcaacagaaatcaggtggtgactacgtgcccgccgag

SEQ ID NO: 91 - DNA, 40 bp, Artificial Sequence
  Description of Artificial Sequence Synthetic probe
agaggactgctaaaggtttgtaggctacgtgcccgccgat

SEQ ID NO: 92 - DNA, 28 bp, Artificial Sequence
  Description of Artificial Sequence Synthetic probe
gagaattagtctgagtctgataactagc

SEQ ID NO: 93 - DNA, 54 bp, Artificial Sequence
  Description of Artificial Sequence Synthetic probe
ctggtccgttgtggtccttctaacggaaaacagggtaatttcaaaaatcttagg

SEQ ID NO: 94 - DNA, 54 bp, Artificial Sequence
  Description of Artificial Sequence Synthetic probe
catttggtcattggttcaagacgaggaaaacagggtaatttcaaaaatcttagt

SEQ ID NO: 95 - DNA, 40 bp, Artificial Sequence
  Description of Artificial Sequence Synthetic probe
gaatttgtgtttaagaatattgatggttattttaaaatat

SEQ ID NO: 96 - DNA, 59 bp, Artificial Sequence
  Description of Artificial Sequence Synthetic probe
ctggtccgttgtggtccttctaactaaaataaccatcaatattcttaaacacaaattcc

SEQ ID NO: 97 - DNA, 59 bp, Artificial Sequence
  Description of Artificial Sequence Synthetic probe
catttggtcattggttcaagacgataaaataaccatcaatattcttaaacacaaattca

SEQ ID NO: 98 - DNA, 30 bp, Artificial Sequence
  Description of Artificial Sequence Synthetic probe
ctaagatttttgaaattaccctgttttcct

SEQ ID NO: 99 - DNA, 47 bp, Artificial Sequence
  Description of Artificial Sequence Synthetic probe
aatccgtcgactagcctgagaattacgcctattaatttagtgcgtga

SEQ ID NO: 100 - DNA, 47 bp, Artificial Sequence
  Description of Artificial Sequence Synthetic probe
ctaatagctcctgtgccctcgtatacgcctattaatttagtgcgtgg

SEQ ID NO: 101 - DNA, 20 bp, Artificial Sequence
  Description of Artificial Sequence Synthetic probe
tctccctcagggtttttcgg

SEQ ID NO: 102 - DNA, 45 bp, Artificial Sequence
  Description of Artificial Sequence Synthetic probe
aatccgtcgactagcctgagaattccgaaaaaccctgagggagat

SEQ ID NO: 103 - DNA, 45 bp, Artificial Sequence
  Description of Artificial Sequence Synthetic probe
ctaatagctcctgtgccctcgtatccgaaaaaccctgagggagac

SEQ ID NO: 104 - DNA, 23 bp, Artificial Sequence
  Description of Artificial Sequence Synthetic probe
cacgcactaaattaataggcgtg

SEQ ID NO: 105 - DNA, 55 bp, Artificial Sequence
  Description of Artificial Sequence Synthetic probe
agaggactgctaaaggtttgtaggattctaaggttggtggtaattataattacct

SEQ ID NO: 106 - DNA, 55 bp, Artificial Sequence
  Description of Artificial Sequence Synthetic probe
gtggcaacagaaatcaggtggtgaattctaaggttggtggtaattataattaccg

SEQ ID NO: 107 - DNA, 32 bp, Artificial Sequence
  Description of Artificial Sequence Synthetic probe
gtatagattgtttaggaagtctaatctcaaac

SEQ ID NO: 108 - DNA, 57 bp, Artificial Sequence
  Description of Artificial Sequence Synthetic probe
agaggactgctaaaggtttgtagggtttgagattagacttcctaaacaatctataca

SEQ ID NO: 109 - DNA, 57 bp, Artificial Sequence
  Description of Artificial Sequence Synthetic probe
gtggcaacagaaatcaggtggtgagtttgagattagacttcctaaacaatctatacc

SEQ ID NO: 110 - DNA, 30 bp, Artificial Sequence
  Description of Artificial Sequence Synthetic probe
ggtaattataattaccaccaaccttagaat

SEQ ID NO: 111 - DNA, 46 bp, Artificial Sequence
  Description of Artificial Sequence Synthetic probe
actggtaacccagacatgatcggtagcacaccttgtaatggtgttg

SEQ ID NO: 112 - DNA, 46 bp, Artificial Sequence
  Description of Artificial Sequence Synthetic probe
actccctgtgggtgagcttaatggagcacaccttgtaatggtgtta

SEQ ID NO: 113 - DNA, 31 bp, Artificial Sequence
  Description of Artificial Sequence Synthetic probe
aaggttttaattgttactttcctttacaatc

SEQ ID NO: 114 - DNA, 57 bp, Artificial Sequence
  Description of Artificial Sequence Synthetic probe
actggtaacccagacatgatcggttgattgtaaaggaaagtaacaattaaaaccttc

SEQ ID NO: 115 - DNA, 57 bp, Artificial Sequence
  Description of Artificial Sequence Synthetic probe
actccctgtgggtgagcttaatggtgattgtaaaggaaagtaacaattaaaaccttt

SEQ ID NO: 116 - DNA, 22 bp, Artificial Sequence
  Description of Artificial Sequence Synthetic probe
aacaccattacaaggtgtgcta

SEQ ID NO: 117 - DNA, 47 bp, Artificial Sequence
  Description of Artificial Sequence Synthetic probe
tagatgccgctgcaggtatggaaagcctacactatgtcacttggtgc

SEQ ID NO: 118 - DNA, 47 bp, Artificial Sequence
  Description of Artificial Sequence Synthetic probe
cgtaccattgaatctggagaccttgcctacactatgtcacttggtgt

SEQ ID NO: 119 - DNA, 31 bp, Artificial Sequence
  Description of Artificial Sequence Synthetic probe
agaaaattcagttgcttactctaataactct

SEQ ID NO: 120 - DNA, 56 bp, Artificial Sequence
  Description of Artificial Sequence Synthetic probe
tagatgccgctgcaggtatggaaaagagttattagagtaagcaactgaattttctg

SEQ ID NO: 121 - DNA, 56 bp, Artificial Sequence
  Description of Artificial Sequence Synthetic probe
cgtaccattgaatctggagaccttagagttattagagtaagcaactgaattttcta

SEQ ID NO: 122 - DNA, 22 bp, Artificial Sequence
  Description of Artificial Sequence Synthetic probe
caccaagtgacatagtgtaggc

SEQ ID NO: 123 - DNA, 45 bp, Artificial Sequence
  Description of Artificial Sequence Synthetic probe
tagatgccgctgcaggtatggaaagctccagggcaaactggaamg

SEQ ID NO: 124 - DNA, 45 bp, Artificial Sequence
  Description of Artificial Sequence Synthetic probe
cgtaccattgaatctggagaccttgctccagggcaaactggaamt

SEQ ID NO: 125 - DNA, 38 bp, Artificial Sequence
  Description of Artificial Sequence Synthetic probe
attgctgattataattataaattaccagatgattttac

SEQ ID NO: 126 - DNA, 63 bp, Artificial Sequence
  Description of Artificial Sequence Synthetic probe
tagatgccgctgcaggtatggaaagtaaaatcatctggtaatttataattataatcagcaatc

SEQ ID NO: 127 - DNA, 63 bp, Artificial Sequence
  Description of Artificial Sequence Synthetic probe
cgtaccattgaatctggagaccttgtaaaatcatctggtaatttataattataatcagcaata

SEQ ID NO: 128 - DNA, 20 bp, Artificial Sequence
  Description of Artificial Sequence Synthetic probe
kttccagtttgccctggagc

SEQ ID NO: 129 - DNA, 44 bp, Artificial Sequence
  Description of Artificial Sequence Synthetic probe
ctggtccgttgtggtccttctaacgctccagggcaaactggaaa

SEQ ID NO: 130 - DNA, 44 bp, Artificial Sequence
  Description of Artificial Sequence Synthetic probe
catttggtcattggttcaagacgagctccagggcaaactggaac

SEQ ID NO: 131 - DNA, 39 bp, Artificial Sequence
  Description of Artificial Sequence Synthetic probe
kattgctgattataattataaattaccagatgattttac

SEQ ID NO: 132 - DNA, 59 bp, Artificial Sequence
  Description of Artificial Sequence Synthetic probe
ctggtccgttgtggtccttctaacatcatctggtaatttataattataatcagcaatmt

SEQ ID NO: 133 - DNA, 59 bp, Artificial Sequence
  Description of Artificial Sequence Synthetic probe
catttggtcattggttcaagacgaatcatctggtaatttataattataatcagcaatmg

SEQ ID NO: 134 - DNA, 19 bp, Artificial Sequence
  Description of Artificial Sequence Synthetic probe
ttccagtttgccctggagc

SEQ ID NO: 135 - DNA, 75 bp, Unknown
  Description of Unknown WT template strand
cctttcttttccaatgttacttggttccatgctatacatgtctctgggaccaatggtactaagaggtttgataac

SEQ ID NO: 136 - DNA, 75 bp, Artificial Sequence
  Description of Artificial Sequence Synthetic oligonucleotide
acctttcttttccaatgttacttggttccatgctatctctgggaccaatggtactaagaggtttgataaccctgt

SEQ ID NO: 137 - DNA, 75 bp, Unknown
  Description of Unknown WT cDNA strand
ttatcaaacctcttagtaccattggtcccagagacatgtatagcatggaaccaagtaacattggaaaagaaaggt

SEQ ID NO: 138 - DNA, 75 bp, Artificial Sequence
  Description of Artificial Sequence Synthetic oligonucleotide
agggttatcaaacctcttagtaccattggtcccagagatagcatggaaccaagtaacattggaaaagaaaggtaa

SEQ ID NO: 139 - DNA, 75 bp, Artificial Sequence
  Description of Artificial Sequence Synthetic oligonucleotide
ttactttcctttacaatcatatggtttccaacccactaatggtgttggttaccaaccatacagagtagtagtact

SEQ ID NO: 140 - DNA, 75 bp, Artificial Sequence
  Description of Artificial Sequence Synthetic oligonucleotide
ttactttcctttacaatcatatggtttccaacccacttatggtgttggttaccaaccatacagagtagtagtact

SEQ ID NO: 141 - DNA, 75 bp, Artificial Sequence
  Description of Artificial Sequence Synthetic oligonucleotide
agtactactactctgtatggttggtaaccaacaccattagtgggttggaaaccatatgattgtaaaggaaagtaa

SEQ ID NO: 142 - DNA, 75 bp, Artificial Sequence
  Description of Artificial Sequence Synthetic oligonucleotide
agtactactactctgtatggttggtaaccaacaccataagtgggttggaaaccatatgattgtaaaggaaagtaa

SEQ ID NO: 143 - DNA, 75 bp, Artificial Sequence
  Description of Artificial Sequence Synthetic oligonucleotide
aggtatatgcgctagttatcagactcagactaattctcctcggcgggcacgtagtgtagctagtcaatccatcat

SEQ ID NO: 144 - DNA, 75 bp, Artificial Sequence
  Description of Artificial Sequence Synthetic oligonucleotide
aggtatatgcgctagttatcagactcagactaattctcatcggcgggcacgtagtgtagctagtcaatccatcat

SEQ ID NO: 145 - DNA, 75 bp, Artificial Sequence
  Description of Artificial Sequence Synthetic oligonucleotide
atgatggattgactagctacactacgtgcccgccgaggagaattagtctgagtctgataactagcgcatatacct

SEQ ID NO: 146 - DNA, 75 bp, Artificial Sequence
  Description of Artificial Sequence Synthetic oligonucleotide
atgatggattgactagctacactacgtgcccgccgatgagaattagtctgagtctgataactagcgcatatacct

SEQ ID NO: 147 - DNA, 78 bp, Artificial Sequence
  Description of Artificial Sequence Synthetic oligonucleotide
accttgaaggaaaacagggtaatttcaaaaatcttagggaatttgtgtttaagaatattgatggttattttaaaatat

SEQ ID NO: 148 - DNA, 78 bp, Artificial Sequence
  Description of Artificial Sequence Synthetic oligonucleotide
accttgaaggaaaacagggtaatttcaaaaatcttagtgaatttgtgtttaagaatattgatggttattttaaaatat

SEQ ID NO: 149 - DNA, 75 bp, Artificial Sequence
  Description of Artificial Sequence Synthetic oligonucleotide
ttttaaaataaccatcaatattcttaaacacaaattccctaagatttttgaaattaccctgttttccttcaaggt

SEQ ID NO: 150 - DNA, 75 bp, Artificial Sequence
  Description of Artificial Sequence Synthetic oligonucleotide
ttttaaaataaccatcaatattcttaaacacaaattcactaagatttttgaaattaccctgttttccttcaaggt

SEQ ID NO: 151 - DNA, 75 bp, Artificial Sequence
  Description of Artificial Sequence Synthetic oligonucleotide
atatattctaagcacacgcctattaatttagtgcgtgatctccctcagggtttttcggctttagaaccattggta

SEQ ID NO: 152 - DNA, 75 bp, Artificial Sequence
  Description of Artificial Sequence Synthetic oligonucleotide
atatattctaagcacacgcctattaatttagtgcgtggtctccctcagggtttttcggctttagaaccattggta

SEQ ID NO: 153 - DNA, 75 bp, Artificial Sequence
  Description of Artificial Sequence Synthetic oligonucleotide
taccaatggttctaaagccgaaaaaccctgagggagatcacgcactaaattaataggcgtgtgcttagaatatat

SEQ ID NO: 154 - DNA, 75 bp, Artificial Sequence
  Description of Artificial Sequence Synthetic oligonucleotide
taccaatggttctaaagccgaaaaaccctgagggagaccacgcactaaattaataggcgtgtgcttagaatatat

SEQ ID NO: 155 - DNA, 75 bp, Artificial Sequence
  Description of Artificial Sequence Synthetic oligonucleotide
aatcttgattctaaggttggtggtaattataattacctgtatagattgtttaggaagtctaatctcaaacctttt

SEQ ID NO: 156 - DNA, 75 bp, Artificial Sequence
  Description of Artificial Sequence Synthetic oligonucleotide
aatcttgattctaaggttggtggtaattataattaccggtatagattgtttaggaagtctaatctcaaacctttt

SEQ ID NO: 157 - DNA, 75 bp, Artificial Sequence
  Description of Artificial Sequence Synthetic oligonucleotide
aaaaggtttgagattagacttcctaaacaatctatacaggtaattataattaccaccaaccttagaatcaagatt

SEQ ID NO: 158 - DNA, 75 bp, Artificial Sequence
  Description of Artificial Sequence Synthetic oligonucleotide
aaaaggtttgagattagacttcctaaacaatctataccggtaattataattaccaccaaccttagaatcaagatt

SEQ ID NO: 159 - DNA, 76 bp, Artificial Sequence
  Description of Artificial Sequence Synthetic oligonucleotide
aatctatcaggccggtagcacaccttgtaatggtgttgaaggttttaattgttactttcctttacaatcatatggt

SEQ ID NO: 160 - DNA, 76 bp, Artificial Sequence
  Description of Artificial Sequence Synthetic oligonucleotide
aatctatcaggccggtagcacaccttgtaatggtgttaaaggttttaattgttactttcctttacaatcatatggt

SEQ ID NO: 161 - DNA, 75 bp, Artificial Sequence
  Description of Artificial Sequence Synthetic oligonucleotide
ccatatgattgtaaaggaaagtaacaattaaaaccttcaacaccattacaaggtgtgctaccggcctgatagatt

SEQ ID NO: 162 - DNA, 75 bp, Artificial Sequence
  Description of Artificial Sequence Synthetic oligonucleotide
ccatatgattgtaaaggaaagtaacaattaaaacctttaacaccattacaaggtgtgctaccggcctgatagatt

SEQ ID NO: 163 - DNA, 75 bp, Artificial Sequence
  Description of Artificial Sequence Synthetic oligonucleotide
agtcaatccatcattgcctacactatgtcacttggtgcagaaaattcagttgcttactctaataactctattgcc

SEQ ID NO: 164 - DNA, 75 bp, Artificial Sequence
  Description of Artificial Sequence Synthetic oligonucleotide
agtcaatccatcattgcctacactatgtcacttggtgtagaaaattcagttgcttactctaataactctattgcc

SEQ ID NO: 165 - DNA, 75 bp, Artificial Sequence
  Description of Artificial Sequence Synthetic oligonucleotide
ggcaatagagttattagagtaagcaactgaattttctgcaccaagtgacatagtgtaggcaatgatggattgact

SEQ ID NO: 166 - DNA, 75 bp, Artificial Sequence
  Description of Artificial Sequence Synthetic oligonucleotide
ggcaatagagttattagagtaagcaactgaattttctacaccaagtgacatagtgtaggcaatgatggattgact

SEQ ID NO: 167 - DNA, 75 bp, Artificial Sequence
  Description of Artificial Sequence Synthetic oligonucleotide
atgaagtcagacaaatcgctccagggcaaactggaamgattgctgattataattataaattaccagatgatttta

SEQ ID NO: 168 - DNA, 75 bp, Artificial Sequence
  Description of Artificial Sequence Synthetic oligonucleotide
atgaagtcagacaaatcgctccagggcaaactggaamtattgctgattataattataaattaccagatgatttta

SEQ ID NO: 169 - DNA, 75 bp, Artificial Sequence
  Description of Artificial Sequence Synthetic oligonucleotide
taaaatcatctggtaatttataattataatcagcaatckttccagtttgccctggagcgatttgtctgacttcat

SEQ ID NO: 170 - DNA, 75 bp, Artificial Sequence
  Description of Artificial Sequence Synthetic oligonucleotide
taaaatcatctggtaatttataattataatcagcaatakttccagtttgccctggagcgatttgtctgacttcat

SEQ ID NO: 171 - DNA, 75 bp, Artificial Sequence
  Description of Artificial Sequence Synthetic oligonucleotide
gatgaagtcagacaaatcgctccagggcaaactggaaakattgctgattataattataaattaccagatgatttt

SEQ ID NO: 172 - DNA, 75 bp, Artificial Sequence
  Description of Artificial Sequence Synthetic oligonucleotide
gatgaagtcagacaaatcgctccagggcaaactggaackattgctgattataattataaattaccagatgatttt

SEQ ID NO: 173 - DNA, 75 bp, Artificial Sequence
  Description of Artificial Sequence Synthetic oligonucleotide
aaaatcatctggtaatttataattataatcagcaatmtttccagtttgccctggagcgatttgtctgacttcatc

SEQ ID NO: 174 - DNA, 75 bp, Artificial Sequence
  Description of Artificial Sequence Synthetic oligonucleotide
aaaatcatctggtaatttataattataatcagcaatmgttccagtttgccctggagcgatttgtctgacttcatc

SEQ ID NO: 175 - DNA, 25 bp, Artificial Sequence
  Description of Artificial Sequence Synthetic oligonucleotide
gttacttggttccatgctatacatg

SEQ ID NO: 176 - DNA, 24 bp, Artificial Sequence
  Description of Artificial Sequence Synthetic oligonucleotide
ccaatgttacttggttccatgcta

SEQ ID NO: 177 - DNA, 23 bp, Artificial Sequence
  Description of Artificial Sequence Synthetic oligonucleotide
tctctgggaccaatggtactaag

SEQ ID NO: 178 - DNA, 22 bp, Artificial Sequence
  Description of Artificial Sequence Synthetic oligonucleotide
ccattggtcccagagacatgta

SEQ ID NO: 179 - DNA, 23 bp, Artificial Sequence
  Description of Artificial Sequence Synthetic oligonucleotide
cttagtaccattggtcccagaga

SEQ ID NO: 180 - DNA, 24 bp, Artificial Sequence
  Description of Artificial Sequence Synthetic oligonucleotide
tagcatggaaccaagtaacattgg

SEQ ID NO: 181 - DNA, 24 bp, Artificial Sequence
  Description of Artificial Sequence Synthetic oligonucleotide
aatcatatggtttccaacccacty

SEQ ID NO: 182 - DNA, 23 bp, Artificial Sequence
  Description of Artificial Sequence Synthetic oligonucleotide
atggtgttggttaccaaccatac

SEQ ID NO: 183 - DNA, 23 bp, Artificial Sequence
  Description of Artificial Sequence Synthetic oligonucleotide
tatggttggtaaccaacaccaty

SEQ ID NO: 184 - DNA, 24 bp, Artificial Sequence
  Description of Artificial Sequence Synthetic oligonucleotide
agtgggttggaaaccatatgattg

SEQ ID NO: 185 - DNA, 28 bp, Artificial Sequence
  Description of Artificial Sequence Synthetic oligonucleotide
ctagttatcagactcagactaattctcm

SEQ ID NO: 186 - DNA, 15 bp, Artificial Sequence
  Description of Artificial Sequence Synthetic oligonucleotide
tcggcgggcacgtag

SEQ ID NO: 187 - DNA, 16 bp, Artificial Sequence
  Description of Artificial Sequence Synthetic oligonucleotide
ctacgtgcccgccgab

SEQ ID NO: 188 - DNA, 28 bp, Artificial Sequence
  Description of Artificial Sequence Synthetic oligonucleotide
gagaattagtctgagtctgataactagc

SEQ ID NO: 189 - DNA, 30 bp, Artificial Sequence
  Description of Artificial Sequence Synthetic oligonucleotide
ggaaaacagggtaatttcaaaaatcttagk

SEQ ID NO: 190 - DNA, 40 bp, Artificial Sequence; identical to SEQ ID NO: 95
  Description of Artificial Sequence Synthetic oligonucleotide

SEQ ID NO: 191 - DNA, 35 bp, Artificial Sequence
  Description of Artificial Sequence Synthetic oligonucleotide
taaaataaccatcaatattcttaaacacaaattcm

SEQ ID NO: 192 - DNA, 30 bp, Artificial Sequence; identical to SEQ ID NO: 98
  Description of Artificial Sequence Synthetic oligonucleotide

SEQ ID NO: 193 - DNA, 23 bp, Artificial Sequence
  Description of Artificial Sequence Synthetic oligonucleotide
acgcctattaatttagtgcgtgr

SEQ ID NO: 194 - DNA, 20 bp, Artificial Sequence
  Description of Artificial Sequence Synthetic oligonucleotide
tctccctcagggtttttcgg

SEQ ID NO: 195 - DNA, 21 bp, Artificial Sequence
  Description of Artificial Sequence Synthetic oligonucleotide
ccgaaaaaccctgagggagay

SEQ ID NO: 196 - DNA, 23 bp, Artificial Sequence
  Description of Artificial Sequence Synthetic oligonucleotide
cacgcactaaattaataggcgtg

SEQ ID NO: 197 - DNA, 31 bp, Artificial Sequence
  Description of Artificial Sequence Synthetic oligonucleotide
attctaaggttggtggtaattataattacck

SEQ ID NO: 198 - DNA, 32 bp, Artificial Sequence; identical to SEQ ID NO: 107
  Description of Artificial Sequence Synthetic oligonucleotide

SEQ ID NO: 199 - DNA, 33 bp, Artificial Sequence
  Description of Artificial Sequence Synthetic oligonucleotide
gtttgagattagacttcctaaacaatctatacm

SEQ ID NO: 200 - DNA, 30 bp, Artificial Sequence; identical to SEQ ID NO: 110
  Description of Artificial Sequence Synthetic oligonucleotide

SEQ ID NO: 201 - DNA, 22 bp, Artificial Sequence
  Description of Artificial Sequence Synthetic oligonucleotide
agcacaccttgtaatggtgttr

SEQ ID NO: 202 - DNA, 31 bp, Artificial Sequence; identical to SEQ ID NO: 113
  Description of Artificial Sequence Synthetic oligonucleotide

SEQ ID NO: 203 - DNA, 33 bp, Artificial Sequence
  Description of Artificial Sequence Synthetic oligonucleotide
tgattgtaaaggaaagtaacaattaaaacctty

SEQ ID NO: 204 - DNA, 22 bp, Artificial Sequence
  Description of Artificial Sequence Synthetic oligonucleotide
aacaccattacaaggtgtgcta

SEQ ID NO: 205 - DNA, 23 bp, Artificial Sequence
  Description of Artificial Sequence Synthetic oligonucleotide
gcctacactatgtcacttggtgy

SEQ ID NO: 206 - DNA, 31 bp, Artificial Sequence; identical to SEQ ID NO: 119
  Description of Artificial Sequence Synthetic oligonucleotide

SEQ ID NO: 207 - DNA, 32 bp, Artificial Sequence
  Description of Artificial Sequence Synthetic oligonucleotide
agagttattagagtaagcaactgaattttctr

SEQ ID NO: 208 - DNA, 22 bp, Artificial Sequence
  Description of Artificial Sequence Synthetic oligonucleotide
caccaagtgacatagtgtaggc

SEQ ID NO: 209 - DNA, 21 bp, Artificial Sequence
  Description of Artificial Sequence Synthetic oligonucleotide
gctccagggcaaactggaamk

SEQ ID NO: 210 - DNA, 38 bp, Artificial Sequence; identical to SEQ ID NO: 125
  Description of Artificial Sequence Synthetic oligonucleotide

SEQ ID NO: 211 - DNA, 39 bp, Artificial Sequence
  Description of Artificial Sequence Synthetic oligonucleotide
gtaaaatcatctggtaatttataattataatcagcaatm

SEQ ID NO: 212 - DNA, 20 bp, Artificial Sequence
  Description of Artificial Sequence Synthetic oligonucleotide
kttccagtttgccctggagc

SEQ ID NO: 213 - DNA, 20 bp, Artificial Sequence
  Description of Artificial Sequence Synthetic oligonucleotide
gctccagggcaaactggaam

SEQ ID NO: 214 - DNA, 39 bp, Artificial Sequence; identical to SEQ ID NO: 131
  Description of Artificial Sequence Synthetic oligonucleotide

SEQ ID NO: 215 - DNA, 35 bp, Artificial Sequence
  Description of Artificial Sequence Synthetic oligonucleotide
atcatctggtaatttataattataatcagcaatmk

SEQ ID NO: 216 - DNA, 19 bp, Artificial Sequence
  Description of Artificial Sequence Synthetic oligonucleotide
ttccagtttgccctggagc

SEQ ID NO: 217 - DNA, 22 bp, Artificial Sequence
  Description of Artificial Sequence Synthetic primer
acgtggtgtttattaccctgac

SEQ ID NO: 218 - DNA, 22 bp, Artificial Sequence
  Description of Artificial Sequence Synthetic primer
gctgcattcagttgaatcacca

SEQ ID NO: 219 - DNA, 25 bp, Artificial Sequence
  Description of Artificial Sequence Synthetic primer
ttccaatgttacttggttccatgct

SEQ ID NO: 220 - DNA, 30 bp, Artificial Sequence
  Description of Artificial Sequence Synthetic primer
gggtatggcaatagagttattagagtaagc

SEQ ID NO: 221 - DNA, 27 bp, Artificial Sequence
  Description of Artificial Sequence Synthetic primer
acattcaactcaggacttgttcttacc

SEQ ID NO: 222 - DNA, 25 bp, Artificial Sequence
  Description of Artificial Sequence Synthetic primer
gctgtccaacctgaagaagaatcac

SEQ ID NO: 223 - DNA, 25 bp, Artificial Sequence
  Description of Artificial Sequence Synthetic primer
caggaagagaatcagcaactgtgtt

SEQ ID NO: 224 - DNA, 27 bp, Artificial Sequence
  Description of Artificial Sequence Synthetic primer
tgtcaagaatctcaagtgtctgtggat

SEQ ID NO: 225 - DNA, 26 bp, Artificial Sequence
  Description of Artificial Sequence Synthetic primer
atcaacttactcctacttggcgtgtt

SEQ ID NO: 226 - DNA, 26 bp, Artificial Sequence
  Description of Artificial Sequence Synthetic primer
tctagcagcaatatcaccaaggcaat

SEQ ID NO: 227 - DNA, 19 bp, Artificial Sequence
  Description of Artificial Sequence Synthetic probe
tccctcagggtttttcggc

SEQ ID NO: 228 - DNA, 19 bp, Artificial Sequence
  Description of Artificial Sequence Synthetic probe
tccctcagggtttttcggt

SEQ ID NO: 229 - DNA, 25 bp, Artificial Sequence
  Description of Artificial Sequence Synthetic probe
tttagaaccattggtagatttgcca

SEQ ID NO: 230 - DNA, 25 bp, Artificial Sequence
  Description of Artificial Sequence Synthetic probe
ggcaaatctaccaatggttctaaag

SEQ ID NO: 231 - DNA, 25 bp, Artificial Sequence
  Description of Artificial Sequence Synthetic probe
ggcaaatctaccaatggttctaaaa

SEQ ID NO: 232 - DNA, 20 bp, Artificial Sequence
  Description of Artificial Sequence Synthetic probe
ccgaaaaaccctgagggaga

SEQ ID NO: 233 - DNA, 18 bp, Artificial Sequence
  Description of Artificial Sequence Synthetic probe
cggtgatgctgctcttgc

SEQ ID NO: 234 - DNA, 18 bp, Artificial Sequence
  Description of Artificial Sequence Synthetic probe
cggtgatgctgctcttgt

SEQ ID NO: 235 - DNA, 22 bp, Artificial Sequence
  Description of Artificial Sequence Synthetic probe
tttgctgctgcttgacagattg

SEQ ID NO: 236 - DNA, 23 bp, Artificial Sequence
  Description of Artificial Sequence Synthetic probe
caatctgtcaagcagcagcaaag

SEQ ID NO: 237 - DNA, 23 bp, Artificial Sequence
  Description of Artificial Sequence Synthetic probe
caatctgtcaagcagcagcaaaa

SEQ ID NO: 238 - DNA, 18 bp, Artificial Sequence
  Description of Artificial Sequence Synthetic probe
caagagcagcatcaccgc

SEQ ID NO: 239 - DNA, 27 bp, Artificial Sequence
  Description of Artificial Sequence Synthetic probe
tgaattctcgtaactacatagcacaag

SEQ ID NO: 240 - DNA, 27 bp, Artificial Sequence
  Description of Artificial Sequence Synthetic probe
tgaattctcgtaactacatagcacaat

SEQ ID NO: 241 - DNA, 30 bp, Artificial Sequence
  Description of Artificial Sequence Synthetic probe
tagatgtagttaactttaatctcacatagc

SEQ ID NO: 242 - DNA, 31 bp, Artificial Sequence
  Description of Artificial Sequence Synthetic probe
gctatgtgagattaaagttaactacatctac

SEQ ID NO: 243 - DNA, 31 bp, Artificial Sequence
  Description of Artificial Sequence Synthetic probe
gctatgtgagattaaagttaactacatctaa

SEQ ID NO: 244 - DNA, 26 bp, Artificial Sequence
  Description of Artificial Sequence Synthetic probe
ttgtgctatgtagttacgagaattca

SEQ ID NO: 245 - DNA, 31 bp, Artificial Sequence
  Description of Artificial Sequence Synthetic probe
ttgaaattaaattggcaaagaaatttgacac

SEQ ID NO: 246 - DNA, 31 bp, Artificial Sequence
  Description of Artificial Sequence Synthetic probe
ttgaaattaaattggcaaagaaatttgacat

SEQ ID NO: 247 - DNA, 26 bp, Artificial Sequence
  Description of Artificial Sequence Synthetic probe
cttcaatggggaatgtccaaattttg

SEQ ID NO: 248 - DNA, 26 bp, Artificial Sequence
  Description of Artificial Sequence Synthetic probe
aaaatttggacattccccattgaagg

SEQ ID NO: 249 - DNA, 26 bp, Artificial Sequence
  Description of Artificial Sequence Synthetic probe
aaaatttggacattccccattgaaga

SEQ ID NO: 250 - DNA, 31 bp, Artificial Sequence
  Description of Artificial Sequence Synthetic probe
tgtcaaatttctttgccaatttaatttcaaa

SEQ ID NO: 251 - DNA, 24 bp, Artificial Sequence
  Description of Artificial Sequence Synthetic probe
gttgcacttcttgctgtttttcag

SEQ ID NO: 252 - DNA, 24 bp, Artificial Sequence
  Description of Artificial Sequence Synthetic probe
gttgcacttcttgctgtttttcat

SEQ ID NO: 253 - DNA, 23 bp, Artificial Sequence
  Description of Artificial Sequence Synthetic probe
agcgcttccaaaatcataaccct

SEQ ID NO: 254 - DNA, 23 bp, Artificial Sequence
  Description of Artificial Sequence Synthetic probe
gggttatgattttggaagcgctc

SEQ ID NO: 255 - DNA, 23 bp, Artificial Sequence
  Description of Artificial Sequence Synthetic probe
gggttatgattttggaagcgcta

SEQ ID NO: 256 - DNA, 23 bp, Artificial Sequence
  Description of Artificial Sequence Synthetic probe
tgaaaaacagcaagaagtgcaac

SEQ ID NO: 257 - DNA, 29 bp, Artificial Sequence
  Description of Artificial Sequence Synthetic probe
ttttgtaatgatccatttttgggtgttta

SEQ ID NO: 258 - DNA, 28 bp, Artificial Sequence
  Description of Artificial Sequence Synthetic probe
aattttgtaatgatccatttttgggtgt

SEQ ID NO: 259 - DNA, 27 bp, Artificial Sequence
  Description of Artificial Sequence Synthetic probe
ttaccacaaaaacaacaaaagttggat

SEQ ID NO: 260 - DNA, 28 bp, Artificial Sequence
  Description of Artificial Sequence Synthetic probe
ccaacttttgttgtttttgtggtaataa

SEQ ID NO: 261 - DNA, 27 bp, Artificial Sequence
  Description of Artificial Sequence Synthetic probe
atccaacttttgttgtttttgtggtaa

SEQ ID NO: 262 - DNA, 28 bp, Artificial Sequence
  Description of Artificial Sequence Synthetic probe
acacccaaaaatggatcattacaaaatt

SEQ ID NO: 263 - DNA, 24 bp, Artificial Sequence
  Description of Artificial Sequence Synthetic probe
ccaacaatttggcagagacattgc

SEQ ID NO: 264 - DNA, 24 bp, Artificial Sequence
  Description of Artificial Sequence Synthetic probe
ccaacaatttggcagagacattga

SEQ ID NO: 265 - DNA, 21 bp, Artificial Sequence
  Description of Artificial Sequence Synthetic probe
tgacactactgatgctgtccg

SEQ ID NO: 266 - DNA, 22 bp, Artificial Sequence
  Description of Artificial Sequence Synthetic probe
cggacagcatcagtagtgtcag

SEQ ID NO: 267 - DNA, 22 bp, Artificial Sequence
  Description of Artificial Sequence Synthetic probe
cggacagcatcagtagtgtcat

SEQ ID NO: 268 - DNA, 24 bp, Artificial Sequence
  Description of Artificial Sequence Synthetic probe
caatgtctctgccaaattgttgga

SEQ ID NO: 269 - DNA, 26 bp, Artificial Sequence
  Description of Artificial Sequence Synthetic probe
tctaataactctattgccatacccac

SEQ ID NO: 270 - DNA, 26 bp, Artificial Sequence
  Description of Artificial Sequence Synthetic probe
tctaataactctattgccatacccat

SEQ ID NO: 271 - DNA, 32 bp, Artificial Sequence
  Description of Artificial Sequence Synthetic probe
aaattttactattagtgttaccacagaaattc

SEQ ID NO: 272 - DNA, 33 bp, Artificial Sequence
  Description of Artificial Sequence Synthetic probe
gaatttctgtggtaacactaatagtaaaatttg

SEQ ID NO: 273 - DNA, 33 bp, Artificial Sequence
  Description of Artificial Sequence Synthetic probe
gaatttctgtggtaacactaatagtaaaattta

SEQ ID NO: 274 - DNA, 29 bp, Artificial Sequence
  Description of Artificial Sequence Synthetic probe
tgggtatggcaatagagttattagagtaa

SEQ ID NO: 275 - DNA, 31 bp, Artificial Sequence
  Description of Artificial Sequence Synthetic probe
gcaatttcaagtgttttaaatgatatccttt

SEQ ID NO: 276 - DNA, 31 bp, Artificial Sequence
  Description of Artificial Sequence Synthetic probe
gcaatttcaagtgttttaaatgatatccttg

SEQ ID NO: 277 - DNA, 22 bp, Artificial Sequence
  Description of Artificial Sequence Synthetic probe
cacgtcttgacaaagttgaggc

SEQ ID NO: 278 - DNA, 23 bp, Artificial Sequence
  Description of Artificial Sequence Synthetic probe
gcctcaactttgtcaagacgtga

SEQ ID NO: 279 - DNA, 23 bp, Artificial Sequence
  Description of Artificial Sequence Synthetic probe
gcctcaactttgtcaagacgtgc

SEQ ID NO: 280 - DNA, 31 bp, Artificial Sequence
  Description of Artificial Sequence Synthetic probe
aaggatatcatttaaaacacttgaaattgca

SEQ ID NO: 281 - DNA, 32 bp, Artificial Sequence
  Description of Artificial Sequence Synthetic probe
gaatttttatgaaccacaaatcattactacag

SEQ ID NO: 282 - DNA, 32 bp, Artificial Sequence
  Description of Artificial Sequence Synthetic probe
gaatttttatgaaccacaaatcattactacac

SEQ ID NO: 283 - DNA, 24 bp, Artificial Sequence
  Description of Artificial Sequence Synthetic probe
acaacacatttgtgtctggtaact

SEQ ID NO: 284 - DNA, 24 bp, Artificial Sequence
  Description of Artificial Sequence Synthetic probe
gttaccagacacaaatgtgttgtc

SEQ ID NO: 285 - DNA, 24 bp, Artificial Sequence
  Description of Artificial Sequence Synthetic probe
gttaccagacacaaatgtgttgtg

SEQ ID NO: 286 - DNA, 32 bp, Artificial Sequence
  Description of Artificial Sequence Synthetic probe
tgtagtaatgatttgtggttcataaaaattcc

SEQ ID NO: 287 - DNA, 24 bp, Artificial Sequence
  Description of Artificial Sequence Synthetic probe
cgatcatcagcacatctaggtttc

SEQ ID NO: 288 - DNA, 24 bp, Artificial Sequence
  Description of Artificial Sequence Synthetic probe
cgatcatcagcacatctaggtttt

SEQ ID NO: 289 - DNA, 19 bp, Artificial Sequence
  Description of Artificial Sequence Synthetic probe
gtccgggtgtgaccgaaag

SEQ ID NO: 290 - DNA, 16 bp, Artificial Sequence
  Description of Artificial Sequence Synthetic probe
cggtcacacccggacg

SEQ ID NO: 291 - DNA, 16 bp, Artificial Sequence
  Description of Artificial Sequence Synthetic probe
cggtcacacccggaca

SEQ ID NO: 292 - DNA, 23 bp, Artificial Sequence
  Description of Artificial Sequence Synthetic probe
aaacctagatgtgctgatgatcg

SEQ ID NO: 293 - DNA, 28 bp, Artificial Sequence
  Description of Artificial Sequence Synthetic probe
attggcttcacatatgtattgttctttc

SEQ ID NO: 294 - DNA, 28 bp, Artificial Sequence
  Description of Artificial Sequence Synthetic probe
attggcttcacatatgtattgttctttt

SEQ ID NO: 295 - DNA, 23 bp, Artificial Sequence
  Description of Artificial Sequence Synthetic probe
taccctccagatgaggatgaaga

SEQ ID NO: 296 - DNA, 23 bp, Artificial Sequence
  Description of Artificial Sequence Synthetic probe
cttcatcctcatctggagggtag

SEQ ID NO: 297 - DNA, 23 bp, Artificial Sequence
  Description of Artificial Sequence Synthetic probe
cttcatcctcatctggagggtaa

SEQ ID NO: 298 - DNA, 28 bp, Artificial Sequence
  Description of Artificial Sequence Synthetic probe
aaagaacaatacatatgtgaagccaatt

SEQ ID NO: 299 - DNA, 25 bp, Artificial Sequence
  Description of Artificial Sequence Synthetic probe
ttttattctctacagtgttcccacc

SEQ ID NO: 300 - DNA, 25 bp, Artificial Sequence
  Description of Artificial Sequence Synthetic probe
ttttattctctacagtgttcccact

SEQ ID NO: 301 - DNA, 25 bp, Artificial Sequence
  Description of Artificial Sequence Synthetic probe
tacaagttttggaccactagtgaga

SEQ ID NO: 302 - DNA, 25 bp, Artificial Sequence
  Description of Artificial Sequence Synthetic probe
ctcactagtggtccaaaacttgtag

SEQ ID NO: 303 - DNA, 25 bp, Artificial Sequence
  Description of Artificial Sequence Synthetic probe
ctcactagtggtccaaaacttgtaa

SEQ ID NO: 304 - DNA, 26 bp, Artificial Sequence
  Description of Artificial Sequence Synthetic probe
gtgggaacactgtagagaataaaact

SEQ ID NO: 305 - DNA, 23 bp, Artificial Sequence
  Description of Artificial Sequence Synthetic probe
gtccaaattcacacaatcgacgg

SEQ ID NO: 306 - DNA, 23 bp, Artificial Sequence
  Description of Artificial Sequence Synthetic probe
gtccaaattcacacaatcgacgt

SEQ ID NO: 307 - DNA, 23 bp, Artificial Sequence
  Description of Artificial Sequence Synthetic probe
ttcatccggagttgttaatccag

SEQ ID NO: 308 - DNA, 24 bp, Artificial Sequence
  Description of Artificial Sequence Synthetic probe
ctggattaacaactccggatgaac

SEQ ID NO: 309 - DNA, 24 bp, Artificial Sequence
  Description of Artificial Sequence Synthetic probe
ctggattaacaactccggatgaaa

SEQ ID NO: 310 - DNA, 23 bp, Artificial Sequence
  Description of Artificial Sequence Synthetic probe
cgtcgattgtgtgaatttggaca

SEQ ID NO: 311 - DNA, 26 bp, Artificial Sequence
  Description of Artificial Sequence Synthetic probe
aagcatacaatgtaacacaagctttc

SEQ ID NO: 312 - DNA, 26 bp, Artificial Sequence
  Description of Artificial Sequence Synthetic probe
aagcatacaatgtaacacaagctttt

SEQ ID NO: 313 - DNA, 18 bp, Artificial Sequence
  Description of Artificial Sequence Synthetic probe
ggcagacgtggtccagaa

SEQ ID NO: 314 - DNA, 17 bp, Artificial Sequence
  Description of Artificial Sequence Synthetic probe
ctggaccacgtctgccg

SEQ ID NO: 315 - DNA, 17 bp, Artificial Sequence
  Description of Artificial Sequence Synthetic probe
ctggaccacgtctgcca

SEQ ID NO: 316 - DNA, 27 bp, Artificial Sequence
  Description of Artificial Sequence Synthetic probe
aaagcttgtgttacattgtatgcttta

SEQ ID NO: 317 - DNA, 27 bp, Artificial Sequence
  Description of Artificial Sequence Synthetic probe
cagatgattttacaggctgcgttatag

SEQ ID NO: 318 - DNA, 27 bp, Artificial Sequence
  Description of Artificial Sequence Synthetic probe
cagatgattttacaggctgcgttatat

SEQ ID NO: 319 - DNA, 30 bp, Artificial Sequence
  Description of Artificial Sequence Synthetic probe
cttggaattctaacaatcttgattctaagg

SEQ ID NO: 320 - DNA, 30 bp, Artificial Sequence
  Description of Artificial Sequence Synthetic probe
cttagaatcaagattgttagaattccaagc

SEQ ID NO: 321 - DNA, 30 bp, Artificial Sequence
  Description of Artificial Sequence Synthetic probe
cttagaatcaagattgttagaattccaaga

SEQ ID NO: 322 - DNA, 25 bp, Artificial Sequence
  Description of Artificial Sequence Synthetic probe
tataacgcagcctgtaaaatcatct

SEQ ID NO: 323 - DNA, 75 bp, Artificial Sequence
  Description of Artificial Sequence Synthetic oligonucleotide
attaatttagtgcgtgatctccctcagggtttttcggctttagaaccattggtagatttgccaataggtattaac

SEQ ID NO: 324 - DNA, 75 bp, Artificial Sequence
  Description of Artificial Sequence Synthetic oligonucleotide
attaatttagtgcgtgatctccctcagggtttttcggttttagaaccattggtagatttgccaataggtattaac

SEQ ID NO: 325 - DNA, 75 bp, Artificial Sequence
  Description of Artificial Sequence Synthetic oligonucleotide
gttaatacctattggcaaatctaccaatggttctaaagccgaaaaaccctgagggagatcacgcactaaattaat

SEQ ID NO: 326 - DNA, 75 bp, Artificial Sequence
  Description of Artificial Sequence Synthetic oligonucleotide
gttaatacctattggcaaatctaccaatggttctaaaaccgaaaaaccctgagggagatcacgcactaaattaat

SEQ ID NO: 327 - DNA, 75 bp, Artificial Sequence
  Description of Artificial Sequence Synthetic oligonucleotide
gctagaatggctggcaatggcggtgatgctgctcttgctttgctgctgcttgacagattgaaccagcttgagagc

SEQ ID NO: 328 - DNA, 75 bp, Artificial Sequence
  Description of Artificial Sequence Synthetic oligonucleotide
gctagaatggctggcaatggcggtgatgctgctcttgttttgctgctgcttgacagattgaaccagcttgagagc

SEQ ID NO: 329 - DNA, 75 bp, Artificial Sequence
  Description of Artificial Sequence Synthetic oligonucleotide
gctctcaagctggttcaatctgtcaagcagcagcaaagcaagagcagcatcaccgccattgccagccattctagc

SEQ ID NO: 330 - DNA, 75 bp, Artificial Sequence
  Description of Artificial Sequence Synthetic oligonucleotide
gctctcaagctggttcaatctgtcaagcagcagcaaaacaagagcagcatcaccgccattgccagccattctagc

SEQ ID NO: 331 - DNA, 75 bp, Artificial Sequence
  Description of Artificial Sequence Synthetic oligonucleotide
cttgtgcagaatgaattctcgtaactacatagcacaagtagatgtagttaactttaatctcacatagcaatcttt

SEQ ID NO: 332 - DNA, 75 bp, Artificial Sequence
  Description of Artificial Sequence Synthetic oligonucleotide
cttgtgcagaatgaattctcgtaactacatagcacaattagatgtagttaactttaatctcacatagcaatcttt

SEQ ID NO: 333 - DNA, 75 bp, Artificial Sequence
  Description of Artificial Sequence Synthetic oligonucleotide
aaagattgctatgtgagattaaagttaactacatctacttgtgctatgtagttacgagaattcattctgcacaag

SEQ ID NO: 334 - DNA, 75 bp, Artificial Sequence
  Description of Artificial Sequence Synthetic oligonucleotide
aaagattgctatgtgagattaaagttaactacatctaattgtgctatgtagttacgagaattcattctgcacaag

SEQ ID NO: 335 - DNA, 75 bp, Artificial Sequence
  Description of Artificial Sequence Synthetic oligonucleotide
acaccttttgaaattaaattggcaaagaaatttgacaccttcaatggggaatgtccaaattttgtatttccctta

SEQ ID NO: 336 - DNA, 75 bp, Artificial Sequence
  Description of Artificial Sequence Synthetic oligonucleotide
acaccttttgaaattaaattggcaaagaaatttgacatcttcaatggggaatgtccaaattttgtatttccctta

SEQ ID NO: 337 - DNA, 75 bp, Artificial Sequence
  Description of Artificial Sequence Synthetic oligonucleotide
taagggaaatacaaaatttggacattccccattgaaggtgtcaaatttctttgccaatttaatttcaaaaggtgt

SEQ ID NO: 338 - DNA, 75 bp, Artificial Sequence
  Description of Artificial Sequence Synthetic oligonucleotide
taagggaaatacaaaatttggacattccccattgaagatgtcaaatttctttgccaatttaatttcaaaaggtgt

SEQ ID NO: 339 - DNA, 75 bp, Artificial Sequence
  Description of Artificial Sequence Synthetic oligonucleotide
ggcttattgttggcgttgcacttcttgctgtttttcagagcgcttccaaaatcataaccctcaaaaagagatggc

SEQ ID NO: 340 - DNA, 75 bp, Artificial Sequence
  Description of Artificial Sequence Synthetic oligonucleotide
ggcttattgttggcgttgcacttcttgctgtttttcatagcgcttccaaaatcataaccctcaaaaagagatggc

SEQ ID NO: 341 - DNA, 75 bp, Artificial Sequence
  Description of Artificial Sequence Synthetic oligonucleotide
gccatctctttttgagggttatgattttggaagcgctctgaaaaacagcaagaagtgcaacgccaacaataagcc

SEQ ID NO: 342 - DNA, 75 bp, Artificial Sequence
  Description of Artificial Sequence Synthetic oligonucleotide
gccatctctttttgagggttatgattttggaagcgctatgaaaaacagcaagaagtgcaacgccaacaataagcc

SEQ ID NO: 343 - DNA, 75 bp, Unknown
  Description of Unknown WT template strand
tgaatttcaattttgtaatgatccatttttgggtgtttattaccacaaaaacaacaaaagttggatggaaagtga

SEQ ID NO: 344 - DNA, 75 bp, Artificial Sequence
  Description of Artificial Sequence Synthetic oligonucleotide
gtgaatttcaattttgtaatgatccatttttgggtgtttaccacaaaaacaacaaaagttggatggaaagtgagt

SEQ ID NO: 345 - DNA, 75 bp, Unknown
  Description of Unknown WT cDNA strand
tcactttccatccaacttttgttgtttttgtggtaataaacacccaaaaatggatcattacaaaattgaaattca

SEQ ID NO: 346 - DNA, 75 bp, Artificial Sequence
  Description of Artificial Sequence Synthetic oligonucleotide
actcactttccatccaacttttgttgtttttgtggtaaacacccaaaaatggatcattacaaaattgaaattcac

SEQ ID NO: 347 - DNA, 75 bp, Artificial Sequence
  Description of Artificial Sequence Synthetic oligonucleotide
aagtttctgcctttccaacaatttggcagagacattgctgacactactgatgctgtccgtgatccacagacactt

SEQ ID NO: 348 - DNA, 75 bp, Artificial Sequence
  Description of Artificial Sequence Synthetic oligonucleotide
aagtttctgcctttccaacaatttggcagagacattgatgacactactgatgctgtccgtgatccacagacactt

SEQ ID NO: 349 - DNA, 75 bp, Artificial Sequence
  Description of Artificial Sequence Synthetic oligonucleotide
aagtgtctgtggatcacggacagcatcagtagtgtcagcaatgtctctgccaaattgttggaaaggcagaaactt

SEQ ID NO: 350 - DNA, 75 bp, Artificial Sequence
  Description of Artificial Sequence Synthetic oligonucleotide
aagtgtctgtggatcacggacagcatcagtagtgtcatcaatgtctctgccaaattgttggaaaggcagaaactt

SEQ ID NO: 351 - DNA, 75 bp, Artificial Sequence
  Description of Artificial Sequence Synthetic oligonucleotide
tcagttgcttactctaataactctattgccatacccacaaattttactattagtgttaccacagaaattctacca

SEQ ID NO: 352 - DNA, 75 bp, Artificial Sequence
  Description of Artificial Sequence Synthetic oligonucleotide
tcagttgcttactctaataactctattgccatacccataaattttactattagtgttaccacagaaattctacca

SEQ ID NO: 353 - DNA, 75 bp, Artificial Sequence
  Description of Artificial Sequence Synthetic oligonucleotide
tggtagaatttctgtggtaacactaatagtaaaatttgtgggtatggcaatagagttattagagtaagcaactga

SEQ ID NO: 354 - DNA, 75 bp, Artificial Sequence
  Description of Artificial Sequence Synthetic oligonucleotide
tggtagaatttctgtggtaacactaatagtaaaatttatgggtatggcaatagagttattagagtaagcaactga

SEQ ID NO: 355 - DNA, 75 bp, Artificial Sequence
  Description of Artificial Sequence Synthetic oligonucleotide
ttttggtgcaatttcaagtgttttaaatgatatcctttcacgtcttgacaaagttgaggctgaagtgcaaatatt

SEQ ID NO: 356 - DNA, 75 bp, Artificial Sequence
  Description of Artificial Sequence Synthetic oligonucleotide
ttttggtgcaatttcaagtgttttaaatgatatccttgcacgtcttgacaaagttgaggctgaagtgcaaatatt

SEQ ID NO: 357 - DNA, 75 bp, Artificial Sequence
  Description of Artificial Sequence Synthetic oligonucleotide
aatatttgcacttcagcctcaactttgtcaagacgtgaaaggatatcatttaaaacacttgaaattgcaccaaaa

SEQ ID NO: 358 - DNA, 75 bp, Artificial Sequence
  Description of Artificial Sequence Synthetic oligonucleotide
aatatttgcacttcagcctcaactttgtcaagacgtgcaaggatatcatttaaaacacttgaaattgcaccaaaa

SEQ ID NO: 359 - DNA, 75 bp, Artificial Sequence
  Description of Artificial Sequence Synthetic oligonucleotide
acaaaggaatttttatgaaccacaaatcattactacagacaacacatttgtgtctggtaactgtgatgttgtaat

SEQ ID NO: 360 - DNA, 75 bp, Artificial Sequence
  Description of Artificial Sequence Synthetic oligonucleotide
acaaaggaatttttatgaaccacaaatcattactacacacaacacatttgtgtctggtaactgtgatgttgtaat

SEQ ID NO: 361 - DNA, 75 bp, Artificial Sequence
  Description of Artificial Sequence Synthetic oligonucleotide
attacaacatcacagttaccagacacaaatgtgttgtctgtagtaatgatttgtggttcataaaaattcctttgt

SEQ ID NO: 362 - DNA, 75 bp, Artificial Sequence
  Description of Artificial Sequence Synthetic oligonucleotide
attacaacatcacagttaccagacacaaatgtgttgtgtgtagtaatgatttgtggttcataaaaattcctttgt

SEQ ID NO: 363 - DNA, 75 bp, Artificial Sequence
  Description of Artificial Sequence Synthetic oligonucleotide
gtccgtgttgcagccgatcatcagcacatctaggtttcgtccgggtgtgaccgaaaggtaagatggagagccttg

SEQ ID NO: 364 - DNA, 75 bp, Artificial Sequence
  Description of Artificial Sequence Synthetic oligonucleotide
gtccgtgttgcagccgatcatcagcacatctaggttttgtccgggtgtgaccgaaaggtaagatggagagccttg

SEQ ID NO: 365 - DNA, 75 bp, Artificial Sequence
  Description of Artificial Sequence Synthetic oligonucleotide
caaggctctccatcttacctttcggtcacacccggacgaaacctagatgtgctgatgatcggctgcaacacggac

SEQ ID NO: 366 - DNA, 75 bp, Artificial Sequence
  Description of Artificial Sequence Synthetic oligonucleotide
caaggctctccatcttacctttcggtcacacccggacaaaacctagatgtgctgatgatcggctgcaacacggac

SEQ ID NO: 367 - DNA, 75 bp, Artificial Sequence
  Description of Artificial Sequence Synthetic oligonucleotide
gtgagtttaaattggcttcacatatgtattgttctttctaccctccagatgaggatgaagaagaaggtgattgtg

SEQ ID NO: 368 - DNA, 75 bp, Artificial Sequence
  Description of Artificial Sequence Synthetic oligonucleotide
gtgagtttaaattggcttcacatatgtattgttctttttaccctccagatgaggatgaagaagaaggtgattgtg

SEQ ID NO: 369 - DNA, 75 bp, Artificial Sequence
  Description of Artificial Sequence Synthetic oligonucleotide
cacaatcaccttcttcttcatcctcatctggagggtagaaagaacaatacatatgtgaagccaatttaaactcac

SEQ ID NO: 370 - DNA, 75 bp, Artificial Sequence
  Description of Artificial Sequence Synthetic oligonucleotide
cacaatcaccttcttcttcatcctcatctggagggtaaaaagaacaatacatatgtgaagccaatttaaactcac

SEQ ID NO: 371 - DNA, 75 bp, Artificial Sequence
  Description of Artificial Sequence Synthetic oligonucleotide
gcaaactttaatgttttattctctacagtgttcccacctacaagttttggaccactagtgagaaaaatatttgtt

SEQ ID NO: 372 - DNA, 75 bp, Artificial Sequence
  Description of Artificial Sequence Synthetic oligonucleotide
gcaaactttaatgttttattctctacagtgttcccacttacaagttttggaccactagtgagaaaaatatttgtt

SEQ ID NO: 373 - DNA, 75 bp, Artificial Sequence
  Description of Artificial Sequence Synthetic oligonucleotide
aacaaatatttttctcactagtggtccaaaacttgtaggtgggaacactgtagagaataaaacattaaagtttgc

SEQ ID NO: 374 - DNA, 75 bp, Artificial Sequence
  Description of Artificial Sequence Synthetic oligonucleotide
aacaaatatttttctcactagtggtccaaaacttgtaagtgggaacactgtagagaataaaacattaaagtttgc

SEQ ID NO: 375 - DNA, 75 bp, Artificial Sequence
  Description of Artificial Sequence Synthetic oligonucleotide
gagcctgaagaacatgtccaaattcacacaatcgacggttcatccggagttgttaatccagtaatggaaccaatt

SEQ ID NO: 376 - DNA, 75 bp, Artificial Sequence
  Description of Artificial Sequence Synthetic oligonucleotide
gagcctgaagaacatgtccaaattcacacaatcgacgtttcatccggagttgttaatccagtaatggaaccaatt

SEQ ID NO: 377 - DNA, 75 bp, Artificial Sequence
  Description of Artificial Sequence Synthetic oligonucleotide
aattggttccattactggattaacaactccggatgaaccgtcgattgtgtgaatttggacatgttcttcaggctc

SEQ ID NO: 378 - DNA, 75 bp, Artificial Sequence
  Description of Artificial Sequence Synthetic oligonucleotide
aattggttccattactggattaacaactccggatgaaacgtcgattgtgtgaatttggacatgttcttcaggctc

SEQ ID NO: 379 - DNA, 75 bp, Artificial Sequence
  Description of Artificial Sequence Synthetic oligonucleotide
gtactgccactaaagcatacaatgtaacacaagctttcggcagacgtggtccagaacaaacccaaggaaattttg

SEQ ID NO: 380 - DNA, 75 bp, Artificial Sequence
  Description of Artificial Sequence Synthetic oligonucleotide
gtactgccactaaagcatacaatgtaacacaagcttttggcagacgtggtccagaacaaacccaaggaaattttg

SEQ ID NO: 381 - DNA, 75 bp, Artificial Sequence
  Description of Artificial Sequence Synthetic oligonucleotide
caaaatttccttgggtttgttctggaccacgtctgccgaaagcttgtgttacattgtatgctttagtggcagtac

SEQ ID NO: 382 - DNA, 75 bp, Artificial Sequence
  Description of Artificial Sequence Synthetic oligonucleotide
caaaatttccttgggtttgttctggaccacgtctgccaaaagcttgtgttacattgtatgctttagtggcagtac

SEQ ID NO: 383 - DNA, 75 bp, Artificial Sequence
  Description of Artificial Sequence Synthetic oligonucleotide
ttataaattaccagatgattttacaggctgcgttatagcttggaattctaacaatcttgattctaaggttggtgg

SEQ ID NO: 384 - DNA, 75 bp, Artificial Sequence
  Description of Artificial Sequence Synthetic oligonucleotide
ttataaattaccagatgattttacaggctgcgttatatcttggaattctaacaatcttgattctaaggttggtgg

SEQ ID NO: 385 - DNA, 75 bp, Artificial Sequence
  Description of Artificial Sequence Synthetic oligonucleotide
ccaccaaccttagaatcaagattgttagaattccaagctataacgcagcctgtaaaatcatctggtaatttataa

SEQ ID NO: 386 - DNA, 75 bp, Artificial Sequence
  Description of Artificial Sequence Synthetic oligonucleotide
ccaccaaccttagaatcaagattgttagaattccaagatataacgcagcctgtaaaatcatctggtaatttataa

SEQ ID NO: 387 - DNA, 19 bp, Artificial Sequence
  Description of Artificial Sequence Synthetic oligonucleotide
tccctcagggtttttcggy

SEQ ID NO: 388 - DNA, 25 bp, Artificial Sequence
  Description of Artificial Sequence Synthetic oligonucleotide
tttagaaccattggtagatttgcca

SEQ ID NO: 389 - DNA, 25 bp, Artificial Sequence
  Description of Artificial Sequence Synthetic oligonucleotide
ggcaaatctaccaatggttctaaar

SEQ ID NO: 390 - DNA, 20 bp, Artificial Sequence
  Description of Artificial Sequence Synthetic oligonucleotide
ccgaaaaaccctgagggaga

SEQ ID NO: 391 - DNA, 18 bp, Artificial Sequence
  Description of Artificial Sequence Synthetic oligonucleotide
cggtgatgctgctcttgy

SEQ ID NO: 392 - DNA, 22 bp, Artificial Sequence
  Description of Artificial Sequence Synthetic oligonucleotide
tttgctgctgcttgacagattg

SEQ ID NO: 393 - DNA, 23 bp, Artificial Sequence
  Description of Artificial Sequence Synthetic oligonucleotide
caatctgtcaagcagcagcaaar

SEQ ID NO: 394 - DNA, 18 bp, Artificial Sequence
  Description of Artificial Sequence Synthetic oligonucleotide
caagagcagcatcaccgc

SEQ ID NO: 395 - DNA, 27 bp, Artificial Sequence
  Description of Artificial Sequence Synthetic oligonucleotide
tgaattctcgtaactacatagcacaak

SEQ ID NO: 396 - DNA, 30 bp, Artificial Sequence; identical to SEQ ID NO: 241
  Description of Artificial Sequence Synthetic oligonucleotide

SEQ ID NO: 397 - DNA, 31 bp, Artificial Sequence
  Description of Artificial Sequence Synthetic oligonucleotide
gctatgtgagattaaagttaactacatctam

SEQ ID NO: 398 - DNA, 26 bp, Artificial Sequence
  Description of Artificial Sequence Synthetic oligonucleotide
ttgtgctatgtagttacgagaattca

SEQ ID NO: 399 - DNA, 31 bp, Artificial Sequence
  Description of Artificial Sequence Synthetic oligonucleotide
ttgaaattaaattggcaaagaaatttgacay

SEQ ID NO: 400 - DNA, 26 bp, Artificial Sequence
  Description of Artificial Sequence Synthetic oligonucleotide
cttcaatggggaatgtccaaattttg

SEQ ID NO: 401 - DNA, 26 bp, Artificial Sequence
  Description of Artificial Sequence Synthetic oligonucleotide
aaaatttggacattccccattgaagr

SEQ ID NO: 402 - DNA, 31 bp, Artificial Sequence; identical to SEQ ID NO: 250
  Description of Artificial Sequence Synthetic oligonucleotide

SEQ ID NO: 403 - DNA, 24 bp, Artificial Sequence
  Description of Artificial Sequence Synthetic oligonucleotide
gttgcacttcttgctgtttttcak

SEQ ID NO: 404 - DNA, 23 bp, Artificial Sequence
  Description of Artificial Sequence Synthetic oligonucleotide
agcgcttccaaaatcataaccct

SEQ ID NO: 405 - DNA, 23 bp, Artificial Sequence
  Description of Artificial Sequence Synthetic oligonucleotide
gggttatgattttggaagcgctm

SEQ ID NO: 406 - DNA, 23 bp, Artificial Sequence
  Description of Artificial Sequence Synthetic oligonucleotide
tgaaaaacagcaagaagtgcaac

SEQ ID NO: 407 - DNA, 29 bp, Artificial Sequence
  Description of Artificial Sequence Synthetic oligonucleotide
ttttgtaatgatccatttttgggtgttta

SEQ ID NO: 408 - DNA, 28 bp, Artificial Sequence
  Description of Artificial Sequence Synthetic oligonucleotide
aattttgtaatgatccatttttgggtgt

SEQ ID NO: 409 - DNA, 27 bp, Artificial Sequence
  Description of Artificial Sequence Synthetic oligonucleotide
ttaccacaaaaacaacaaaagttggat

SEQ ID NO: 410 - DNA, 28 bp, Artificial Sequence
  Description of Artificial Sequence Synthetic oligonucleotide
ccaacttttgttgtttttgtggtaataa

SEQ ID NO: 411 - DNA, 27 bp, Artificial Sequence
  Description of Artificial Sequence Synthetic oligonucleotide
atccaacttttgttgtttttgtggtaa

SEQ ID NO: 412 - DNA, 28 bp, Artificial Sequence
  Description of Artificial Sequence Synthetic oligonucleotide
acacccaaaaatggatcattacaaaatt

SEQ ID NO: 413 - DNA, 24 bp, Artificial Sequence
  Description of Artificial Sequence Synthetic oligonucleotide
ccaacaatttggcagagacattgm

SEQ ID NO: 414 - DNA, 21 bp, Artificial Sequence
  Description of Artificial Sequence Synthetic oligonucleotide
tgacactactgatgctgtccg

SEQ ID NO: 415 - DNA, 22 bp, Artificial Sequence
  Description of Artificial Sequence Synthetic oligonucleotide
cggacagcatcagtagtgtcak

SEQ ID NO: 416 - DNA, 24 bp, Artificial Sequence
  Description of Artificial Sequence Synthetic oligonucleotide
caatgtctctgccaaattgttgga

SEQ ID NO: 417 - DNA, 26 bp, Artificial Sequence
  Description of Artificial Sequence Synthetic oligonucleotide
tctaataactctattgccatacccay

SEQ ID NO: 418 - DNA, 32 bp, Artificial Sequence; identical to SEQ ID NO: 271
  Description of Artificial Sequence Synthetic oligonucleotide

SEQ ID NO: 419 - DNA, 33 bp, Artificial Sequence
  Description of Artificial Sequence Synthetic oligonucleotide
gaatttctgtggtaacactaatagtaaaatttr

SEQ ID NO: 420 - DNA, 29 bp, Artificial Sequence
  Description of Artificial Sequence Synthetic oligonucleotide
tgggtatggcaatagagttattagagtaa

SEQ ID NO: 421 - DNA, 31 bp, Artificial Sequence
  Description of Artificial Sequence Synthetic oligonucleotide
gcaatttcaagtgttttaaatgatatccttk

SEQ ID NO: 422 - DNA, 22 bp, Artificial Sequence
  Description of Artificial Sequence Synthetic oligonucleotide
cacgtcttgacaaagttgaggc

SEQ ID NO: 423 - DNA, 23 bp, Artificial Sequence
  Description of Artificial Sequence Synthetic oligonucleotide
gcctcaactttgtcaagacgtgm

SEQ ID NO: 424 - DNA, 31 bp, Artificial Sequence; identical to SEQ ID NO: 280
  Description of Artificial Sequence Synthetic oligonucleotide

SEQ ID NO: 425 - DNA, 32 bp, Artificial Sequence
  Description of Artificial Sequence Synthetic oligonucleotide
gaatttttatgaaccacaaatcattactacas

SEQ ID NO: 426 - DNA, 24 bp, Artificial Sequence
  Description of Artificial Sequence Synthetic oligonucleotide
acaacacatttgtgtctggtaact

SEQ ID NO: 427 - DNA, 24 bp, Artificial Sequence
  Description of Artificial Sequence Synthetic oligonucleotide
gttaccagacacaaatgtgttgts

SEQ ID NO: 428 - DNA, 32 bp, Artificial Sequence; identical to SEQ ID NO: 286
  Description of Artificial Sequence Synthetic oligonucleotide

SEQ ID NO: 429 - DNA, 24 bp, Artificial Sequence
  Description of Artificial Sequence Synthetic oligonucleotide
cgatcatcagcacatctaggttty

SEQ ID NO: 430 - DNA, 19 bp, Artificial Sequence
  Description of Artificial Sequence Synthetic oligonucleotide
gtccgggtgtgaccgaaag

SEQ ID NO: 431 - DNA, 16 bp, Artificial Sequence
  Description of Artificial Sequence Synthetic oligonucleotide
cggtcacacccggacr

SEQ ID NO: 432 - DNA, 23 bp, Artificial Sequence
  Description of Artificial Sequence Synthetic oligonucleotide
aaacctagatgtgctgatgatcg

SEQ ID NO: 433 - DNA, 28 bp, Artificial Sequence
  Description of Artificial Sequence Synthetic oligonucleotide
attggcttcacatatgtattgttcttty

SEQ ID NO: 434 - DNA, 23 bp, Artificial Sequence
  Description of Artificial Sequence Synthetic oligonucleotide
taccctccagatgaggatgaaga

SEQ ID NO: 435 - DNA, 23 bp, Artificial Sequence
  Description of Artificial Sequence Synthetic oligonucleotide
cttcatcctcatctggagggtar

SEQ ID NO: 436 - DNA, 28 bp, Artificial Sequence
  Description of Artificial Sequence Synthetic oligonucleotide
aaagaacaatacatatgtgaagccaatt

SEQ ID NO: 437 - DNA, 25 bp, Artificial Sequence
  Description of Artificial Sequence Synthetic oligonucleotide
ttttattctctacagtgttcccacy

SEQ ID NO: 438 - DNA, 25 bp, Artificial Sequence
  Description of Artificial Sequence Synthetic oligonucleotide
tacaagttttggaccactagtgaga

SEQ ID NO: 439 - DNA, 25 bp, Artificial Sequence
  Description of Artificial Sequence Synthetic oligonucleotide
ctcactagtggtccaaaacttgtar

SEQ ID NO: 440 - DNA, 26 bp, Artificial Sequence
  Description of Artificial Sequence Synthetic oligonucleotide
gtgggaacactgtagagaataaaact

SEQ ID NO: 441 - DNA, 23 bp, Artificial Sequence
  Description of Artificial Sequence Synthetic oligonucleotide
gtccaaattcacacaatcgacgk

SEQ ID NO: 442 - DNA, 23 bp, Artificial Sequence
  Description of Artificial Sequence Synthetic oligonucleotide
ttcatccggagttgttaatccag

SEQ ID NO: 443 - DNA, 24 bp, Artificial Sequence
  Description of Artificial Sequence Synthetic oligonucleotide
ctggattaacaactccggatgaam

SEQ ID NO: 444 - DNA, 23 bp, Artificial Sequence
  Description of Artificial Sequence Synthetic oligonucleotide
cgtcgattgtgtgaatttggaca

SEQ ID NO: 445 - DNA, 26 bp, Artificial Sequence
  Description of Artificial Sequence Synthetic oligonucleotide
aagcatacaatgtaacacaagcttty

SEQ ID NO: 446 - DNA, 18 bp, Artificial Sequence
  Description of Artificial Sequence Synthetic oligonucleotide
ggcagacgtggtccagaa

SEQ ID NO: 447 - DNA, 17 bp, Artificial Sequence
  Description of Artificial Sequence Synthetic oligonucleotide
ctggaccacgtctgccr

SEQ ID NO: 448 - DNA, 27 bp, Artificial Sequence
  Description of Artificial Sequence Synthetic oligonucleotide
aaagcttgtgttacattgtatgcttta

SEQ ID NO: 449 - DNA, 27 bp, Artificial Sequence
  Description of Artificial Sequence Synthetic oligonucleotide
cagatgattttacaggctgcgttatak

SEQ ID NO: 450 - DNA, 30 bp, Artificial Sequence; identical to SEQ ID NO: 319
  Description of Artificial Sequence Synthetic oligonucleotide

SEQ ID NO: 451 - DNA, 30 bp, Artificial Sequence
  Description of Artificial Sequence Synthetic oligonucleotide
cttagaatcaagattgttagaattccaagm

SEQ ID NO: 452 - DNA, 25 bp, Artificial Sequence
  Description of Artificial Sequence Synthetic oligonucleotide
tataacgcagcctgtaaaatcatct

SEQ ID NO: 453 - DNA, 22 bp, Artificial Sequence
  Description of Artificial Sequence Synthetic primer
cgtggtgtttattaccctgaca

SEQ ID NO: 454 - DNA, 22 bp, Artificial Sequence
  Description of Artificial Sequence Synthetic primer
ataagtagggactgggtcttcg

SEQ ID NO: 455 - DNA, 22 bp, Artificial Sequence
  Description of Artificial Sequence Synthetic primer
ggatggaaagtgagttcagagt

SEQ ID NO: 456 - DNA, 22 bp, Artificial Sequence
  Description of Artificial Sequence Synthetic primer
gctgtccaacctgaagaagaat

SEQ ID NO: 457 - DNA, 22 bp, Artificial Sequence
  Description of Artificial Sequence Synthetic primer
caggctgcgttatagcttggaa

SEQ ID NO: 458 - DNA, 22 bp, Artificial Sequence
  Description of Artificial Sequence Synthetic primer
tgctggtgcatgtagaagttca

SEQ ID NO: 459 - DNA, 22 bp, Artificial Sequence
  Description of Artificial Sequence Synthetic primer
tgagtgtgacatacccattggt

SEQ ID NO: 460 - DNA, 22 bp, Artificial Sequence
  Description of Artificial Sequence Synthetic primer
tgctgcattcagttgaatcacc

SEQ ID NO: 461 - DNA, 27 bp, Artificial Sequence
  Description of Artificial Sequence Synthetic primer
acattcaactcaggacttgttcttacc

SEQ ID NO: 462 - DNA, 30 bp, Artificial Sequence
  Description of Artificial Sequence Synthetic primer
cagcctcttattatgttagacttctcagtg

SEQ ID NO: 463 - DNA, 27 bp, Artificial Sequence
  Description of Artificial Sequence Synthetic primer
gttggatggaaagtgagttcagagttt

SEQ ID NO: 464 - DNA, 27 bp, Artificial Sequence
  Description of Artificial Sequence Synthetic primer
tggcaaatctaccaatggttctaaagc

SEQ ID NO: 465 - DNA, 25 bp, Artificial Sequence
  Description of Artificial Sequence Synthetic primer
caggaagagaatcagcaactgtgtt

SEQ ID NO: 466 - DNA, 24 bp, Artificial Sequence
  Description of Artificial Sequence Synthetic primer
tccaagctataacgcagcctgtaa

SEQ ID NO: 467 - DNA, 25 bp, Artificial Sequence
  Description of Artificial Sequence Synthetic primer
caatcttgattctaaggttggtggt

SEQ ID NO: 468 - DNA, 27 bp, Artificial Sequence
  Description of Artificial Sequence Synthetic primer
gtactactactctgtatggttggtaac

SEQ ID NO: 469 - DNA, 27 bp, Artificial Sequence
  Description of Artificial Sequence Synthetic primer
tccacagacacttgagattcttgacat

SEQ ID NO: 470 - DNA, 26 bp, Artificial Sequence
  Description of Artificial Sequence Synthetic primer
aacacgccaagtaggagtaagttgat

SEQ ID NO: 471 - DNA, 31 bp, Artificial Sequence
  Description of Artificial Sequence Synthetic primer
gctagttatcagactcagactaattctcctc

SEQ ID NO: 472 - DNA, 26 bp, Artificial Sequence
  Description of Artificial Sequence Synthetic primer
attgctgcattcagttgaatcaccac

SEQ ID NO: 473 - DNA, 30 bp, Artificial Sequence
  Description of Artificial Sequence Synthetic primer
gtcagtgtgttaatcttacaaccagaactc

SEQ ID NO: 474 - DNA, 30 bp, Artificial Sequence
  Description of Artificial Sequence Synthetic primer
gcctcttattatgttagacttctcagtgga

SEQ ID NO: 475 - DNA, 25 bp, Artificial Sequence
  Description of Artificial Sequence Synthetic primer
tggatggaaagtgagttcagagttt

SEQ ID NO: 476 - DNA, 25 bp, Artificial Sequence
  Description of Artificial Sequence Synthetic primer
ctgaagaagaatcaccaggagtcaa

SEQ ID NO: 477 - DNA, 24 bp, Artificial Sequence
  Description of Artificial Sequence Synthetic primer
tacaggctgcgttatagcttggaa

SEQ ID NO: 478 - DNA, 24 bp, Artificial Sequence
  Description of Artificial Sequence Synthetic primer
aacagttgctggtgcatgtagaag

SEQ ID NO: 479 - DNA, 28 bp, Artificial Sequence
  Description of Artificial Sequence Synthetic primer
tcatgcagatcaacttactcctacttgg

SEQ ID NO: 480 - DNA, 30 bp, Artificial Sequence
  Description of Artificial Sequence Synthetic primer
catagacactggtagaatttctgtggtaac

SEQ ID NO: 481 - DNA, 20 bp, Artificial Sequence
  Description of Artificial Sequence Synthetic primer
aacgccaccagatttgcatc

SEQ ID NO: 482 - DNA, 21 bp, Artificial Sequence
  Description of Artificial Sequence Synthetic primer
acgcagcctgtaaaatcatct

SEQ ID NO: 483 - DNA, 23 bp, Artificial Sequence
  Description of Artificial Sequence Synthetic primer
actgtgttgctgattattctgtc

SEQ ID NO: 484 - DNA, 23 bp, Artificial Sequence
  Description of Artificial Sequence Synthetic primer
ctataacgcagcctgtaaaatca

SEQ ID NO: 485 - DNA, 20 bp, Artificial Sequence
  Description of Artificial Sequence Synthetic primer
aaacttgtgcccttttggtg

SEQ ID NO: 486 - DNA, 21 bp, Artificial Sequence
  Description of Artificial Sequence Synthetic primer
aggggtactgctgttatgtct

SEQ ID NO: 487 - DNA, 20 bp, Artificial Sequence
  Description of Artificial Sequence Synthetic primer
gcagtagcgcgaacaaaatc

SEQ ID NO: 488 - DNA, 25 bp, Artificial Sequence
  Description of Artificial Sequence Synthetic primer
atttgacatgagtaaatttcccctt

SEQ ID NO: 489 - DNA, 21 bp, Artificial Sequence
  Description of Artificial Sequence Synthetic primer
gcatccttgatttcaccttgc

SEQ ID NO: 490 - DNA, 26 bp, Artificial Sequence
  Description of Artificial Sequence Synthetic primer
ttaaattaaggggtactgctgttatg

SEQ ID NO: 491 - DNA, 22 bp, Artificial Sequence
  Description of Artificial Sequence Synthetic primer
cgcgaacaaaatctgaaggagt

SEQ ID NO: 492 - DNA, 48 bp, Artificial Sequence
  Description of Artificial Sequence Synthetic probe
actggtaacccagacatgatcggtgctgattttgacacatggtttagt

SEQ ID NO: 493 - DNA, 48 bp, Artificial Sequence
  Description of Artificial Sequence Synthetic probe
actccctgtgggtgagcttaatgggctgattttgacacatggtttagc

SEQ ID NO: 494 - DNA, 21 bp, Artificial Sequence
  Description of Artificial Sequence Synthetic probe
cagcgtggtggtagttatact

SEQ ID NO: 495 - DNA, 53 bp, Artificial Sequence
  Description of Artificial Sequence Synthetic probe
catttggtcattggttcaagacgagatatcggtaattatacagtttcctgttc

SEQ ID NO: 496 - DNA, 53 bp, Artificial Sequence
  Description of Artificial Sequence Synthetic probe
ctggtccgttgtggtccttctaacgatatcggtaattatacagtttcctgttt

SEQ ID NO: 497 - DNA, 26 bp, Artificial Sequence
  Description of Artificial Sequence Synthetic probe
accttttacaattaattgccaggaac

SEQ ID NO: 498 - DNA, 47 bp, Artificial Sequence
  Description of Artificial Sequence Synthetic probe
ctaatagctcctgtgccctcgtatcaggttgctgttctttatcagga

SEQ ID NO: 499 - DNA, 47 bp, Artificial Sequence
  Description of Artificial Sequence Synthetic probe
aatccgtcgactagcctgagaattcaggttgctgttctttatcaggg

SEQ ID NO: 500 - DNA, 22 bp, Artificial Sequence
  Description of Artificial Sequence Synthetic probe
tgttaactgcacagaagtccct

SEQ ID NO: 501 - DNA, 54 bp, Artificial Sequence
  Description of Artificial Sequence Synthetic probe
gtggcaacagaaatcaggtggtgaagtactcaatggtctttgttcttttttttg

SEQ ID NO: 502 - DNA, 54 bp, Artificial Sequence
  Description of Artificial Sequence Synthetic probe
agaggactgctaaaggtttgtaggagtactcaatggtctttgttcttttttttt

SEQ ID NO: 503 - DNA, 28 bp, Artificial Sequence
  Description of Artificial Sequence Synthetic probe
tatgaaaatgcctttttaccttttgcta

SEQ ID NO: 504 - DNA, 51 bp, Artificial Sequence
  Description of Artificial Sequence Synthetic probe
gtggcaacagaaatcaggtggtgagcaaaaggtaaaaaggcattttcatac

SEQ ID NO: 505 - DNA, 51 bp, Artificial Sequence
  Description of Artificial Sequence Synthetic probe
agaggactgctaaaggtttgtagggcaaaaggtaaaaaggcattttcataa

SEQ ID NO: 506 - DNA, 30 bp, Artificial Sequence
  Description of Artificial Sequence Synthetic probe
aaaaaaaagaacaaagaccattgagtactc

SEQ ID NO: 507 - DNA, 75 bp, Artificial Sequence
  Description of Artificial Sequence Synthetic oligonucleotide
ttgctaacaaacatgctgattttgacacatggtttagtcagcgtggtggtagttatactaatgacaaagcttgcc

SEQ ID NO: 508 - DNA, 75 bp, Artificial Sequence
  Description of Artificial Sequence Synthetic oligonucleotide
ttgctaacaaacatgctgattttgacacatggtttagccagcgtggtggtagttatactaatgacaaagcttgcc

SEQ ID NO: 509 - DNA, 75 bp, Artificial Sequence
  Description of Artificial Sequence Synthetic oligonucleotide
ggcaagctttgtcattagtataactaccaccacgctgactaaaccatgtgtcaaaatcagcatgtttgttagcaa

SEQ ID NO: 510 - DNA, 75 bp, Artificial Sequence
  Description of Artificial Sequence Synthetic oligonucleotide
ggcaagctttgtcattagtataactaccaccacgctggctaaaccatgtgtcaaaatcagcatgtttgttagcaa

SEQ ID NO: 511 - DNA, 75 bp, Artificial Sequence
  Description of Artificial Sequence Synthetic oligonucleotide
cagtacatcgatatcggtaattatacagtttcctgttcaccttttacaattaattgccaggaacctaaattgggt

SEQ ID NO: 512 - DNA, 75 bp, Artificial Sequence
  Description of Artificial Sequence Synthetic oligonucleotide
cagtacatcgatatcggtaattatacagtttcctgtttaccttttacaattaattgccaggaacctaaattgggt

SEQ ID NO: 513 - DNA, 75 bp, Artificial Sequence
  Description of Artificial Sequence Synthetic oligonucleotide
acccaatttaggttcctggcaattaattgtaaaaggtgaacaggaaactgtataattaccgatatcgatgtactg

SEQ ID NO: 514 - DNA, 75 bp, Artificial Sequence
  Description of Artificial Sequence Synthetic oligonucleotide
acccaatttaggttcctggcaattaattgtaaaaggtaaacaggaaactgtataattaccgatatcgatgtactg

SEQ ID NO: 515 - DNA, 75 bp, Artificial Sequence
  Description of Artificial Sequence Synthetic oligonucleotide
acaaatacttctaaccaggttgctgttctttatcaggatgttaactgcacagaagtccctgttgctattcatgca

SEQ ID NO: 516 - DNA, 75 bp, Artificial Sequence
  Description of Artificial Sequence Synthetic oligonucleotide
acaaatacttctaaccaggttgctgttctttatcagggtgttaactgcacagaagtccctgttgctattcatgca

SEQ ID NO: 517 - DNA, 75 bp, Artificial Sequence
  Description of Artificial Sequence Synthetic oligonucleotide
tgcatgaatagcaacagggacttctgtgcagttaacatcctgataaagaacagcaacctggttagaagtatttgt

SEQ ID NO: 518 - DNA, 75 bp, Artificial Sequence
  Description of Artificial Sequence Synthetic oligonucleotide
tgcatgaatagcaacagggacttctgtgcagttaacaccctgataaagaacagcaacctggttagaagtatttgt

SEQ ID NO: 519 - DNA, 75 bp, Artificial Sequence
  Description of Artificial Sequence Synthetic oligonucleotide
tactccagagtactcaatggtctttgttcttttttttgtatgaaaatgcctttttaccttttgctatgggtatta

SEQ ID NO: 520 - DNA, 75 bp, Artificial Sequence
  Description of Artificial Sequence Synthetic oligonucleotide
tactccagagtactcaatggtctttgttcttttttttttatgaaaatgcctttttaccttttgctatgggtatta

SEQ ID NO: 521 - DNA, 75 bp, Artificial Sequence
  Description of Artificial Sequence Synthetic oligonucleotide
taatacccatagcaaaaggtaaaaaggcattttcatacaaaaaaaagaacaaagaccattgagtactctggacta

SEQ ID NO: 522 - DNA, 75 bp, Artificial Sequence
  Description of Artificial Sequence Synthetic oligonucleotide
taatacccatagcaaaaggtaaaaaggcattttcataaaaaaaaaagaacaaagaccattgagtactctggacta

SEQ ID NO: 523 - DNA, 24 bp, Artificial Sequence
  Description of Artificial Sequence Synthetic oligonucleotide
gctgattttgacacatggtttagy

SEQ ID NO: 524 - DNA, 21 bp, Artificial Sequence
  Description of Artificial Sequence Synthetic oligonucleotide
cagcgtggtggtagttatact

SEQ ID NO: 525 - DNA, 29 bp, Artificial Sequence
  Description of Artificial Sequence Synthetic oligonucleotide
gatatcggtaattatacagtttcctgtty

SEQ ID NO: 526 - DNA, 26 bp, Artificial Sequence
  Description of Artificial Sequence Synthetic oligonucleotide
accttttacaattaattgccaggaac

SEQ ID NO: 527 - DNA, 23 bp, Artificial Sequence
  Description of Artificial Sequence Synthetic oligonucleotide
caggttgctgttctttatcaggr

SEQ ID NO: 528 - DNA, 22 bp, Artificial Sequence
  Description of Artificial Sequence Synthetic oligonucleotide
tgttaactgcacagaagtccct

SEQ ID NO: 529 - DNA, 30 bp, Artificial Sequence
  Description of Artificial Sequence Synthetic oligonucleotide
agtactcaatggtctttgttcttttttttk

SEQ ID NO: 530 - DNA, 28 bp, Artificial Sequence
  Description of Artificial Sequence Synthetic oligonucleotide
tatgaaaatgcctttttaccttttgcta

SEQ ID NO: 531 - DNA, 27 bp, Artificial Sequence
  Description of Artificial Sequence Synthetic oligonucleotide
gcaaaaggtaaaaaggcattttcatam

SEQ ID NO: 532 - DNA, 30 bp, Artificial Sequence; identical to SEQ ID NO: 506
  Description of Artificial Sequence Synthetic oligonucleotide

SEQ ID NO: 533 - DNA, 51 bp, Artificial Sequence
  Description of Artificial Sequence Synthetic probe
cgtaccattgaatctggagaccttacatagaagttatttgactcctggtga

SEQ ID NO: 534 - DNA, 51 bp, Artificial Sequence
  Description of Artificial Sequence Synthetic probe
tagatgccgctgcaggtatggaaaacatagaagttatttgactcctggtgg

SEQ ID NO: 535 - DNA, 23 bp, Artificial Sequence
  Description of Artificial Sequence Synthetic probe
ttcttcttcaggttggacagctg

SEQ ID NO: 536 - DNA, 47 bp, Artificial Sequence
  Description of Artificial Sequence Synthetic probe
cgtaccattgaatctggagaccttagctgtccaacctgaagaagaat

SEQ ID NO: 537 - DNA, 47 bp, Artificial Sequence
  Description of Artificial Sequence Synthetic probe
tagatgccgctgcaggtatggaaaagctgtccaacctgaagaagaac

SEQ ID NO: 538 - DNA, 29 bp, Artificial Sequence
  Description of Artificial Sequence Synthetic probe
caccaggagtcaaataacttctatgtaaa

SEQ ID NO: 539 - DNA, 75 bp, Artificial Sequence
  Description of Artificial Sequence Synthetic oligonucleotide
ttacttgctttacatagaagttatttgactcctggtgattcttcttcaggttggacagctggtgctgcagcttat

SEQ ID NO: 540 - DNA, 75 bp, Artificial Sequence
  Description of Artificial Sequence Synthetic oligonucleotide
ttacttgctttacatagaagttatttgactcctggtggttcttcttcaggttggacagctggtgctgcagcttat

SEQ ID NO: 541 - DNA, 75 bp, Artificial Sequence
  Description of Artificial Sequence Synthetic oligonucleotide
ataagctgcagcaccagctgtccaacctgaagaagaatcaccaggagtcaaataacttctatgtaaagcaagtaa

SEQ ID NO: 542 - DNA, 75 bp, Artificial Sequence
  Description of Artificial Sequence Synthetic oligonucleotide
ataagctgcagcaccagctgtccaacctgaagaagaaccaccaggagtcaaataacttctatgtaaagcaagtaa

SEQ ID NO: 543 - DNA, 27 bp, Artificial Sequence
  Description of Artificial Sequence Synthetic oligonucleotide
acatagaagttatttgactcctggtgr

SEQ ID NO: 544 - DNA, 23 bp, Artificial Sequence
  Description of Artificial Sequence Synthetic oligonucleotide
ttcttcttcaggttggacagctg

SEQ ID NO: 545 - DNA, 23 bp, Artificial Sequence
  Description of Artificial Sequence Synthetic oligonucleotide
agctgtccaacctgaagaagaay

SEQ ID NO: 546 - DNA, 29 bp, Artificial Sequence
  Description of Artificial Sequence Synthetic oligonucleotide
caccaggagtcaaataacttctatgtaaa

SEQ ID NO: 547 - protein, 6 aa, Artificial Sequence
  Description of Artificial Sequence Synthetic 6xHis tag
HHHHHH

SEQ ID NO: 548 - protein, 8 aa, Artificial Sequence
  Description of Artificial Sequence Synthetic peptide
DYKDDDDK

(19) United States
(12) Patent Application Publication
(10) Pub. No.: US 2022/0003766 A1
(43) Pub. Date: Jan. 6, 2022

(54) METHODS AND KITS FOR VIRUS DETECTION

(71) Applicant: Meso Scale Technologies, LLC., Rockville, MD (US)

(72) Inventors: Jacob N. WOHLSTADTER, Potomac, MD (US); George SIGAL, Rockville, MD (US); Anu MATHEW, North Potomac, MD (US); Anastasia AKSYUK, Rockville, MD (US); David ROUTENBERG, Gaithersburg, MD (US); Martin STENGELIN, Gaithersburg, MD (US); John KENTEN, Boyds, MD (US); Timothy J. BREAK, Clarksburg, MD (US); Seth B. HARKINS, Bethesda, MD (US); Michael TSIONSKY, Derwood, MD (US)

(21) Appl. No.: 17/246,360

(22) Filed: Apr. 30, 2021

Related U.S. Application Data
(60) Provisional application No. 63/174,247, filed on Apr. 13, 2021.
(60) Provisional application No. 63/168,367, filed on Mar. 31, 2021.
(60) Provisional application No. 63/161,019, filed on Mar. 15, 2021.
(60) Provisional application No. 63/152,648, filed on Feb. 23, 2021.
(60) Provisional application No. 63/128,356, filed on Dec. 21, 2020.
(60) Provisional application No. 63/123,108, filed on Dec. 9, 2020.
(60) Provisional application No. 63/087,956, filed on Oct. 6, 2020.
(60) Provisional application No. 63/074,073, filed on Sep. 3, 2020.
(60) Provisional application No. 63/062,175, filed on Aug. 6, 2020.
(60) Provisional application No. 63/047,644, filed on Jul. 2, 2020.
(60) Provisional application No. 63/039,890, filed on Jun. 16, 2020.
(60) Provisional application No. 63/034,177, filed on Jun. 3, 2020.
(60) Provisional application No. 63/032,031, filed on May 29, 2020.
(60) Provisional application No. 63/027,234, filed on May 19, 2020.
(60) Provisional application No. 63/025,609, filed on May 15, 2020.
(60) Provisional application No. 63/018,893, filed on May 1, 2020.

(51) Int. Cl. G01N 33/569; C12Q 1/70

(52) U.S. Cl. CPC G01N 33/56983; G01N 2333/165; C12Q 1/701

(57) ABSTRACT

The invention relates to methods and kits for detecting a virus, e.g., a respiratory virus such as a coronavirus, in a biological sample. The invention also relates to methods and kits for detecting and/or quantifying biomarkers, e.g., antibody biomarkers against a viral antigen; inflammatory and/or tissue damage response biomarkers; and/or extracellular vesicles in response to a viral infection.

### SEQUENCE LISTING

The instant application contains a Sequence Listing which has been submitted electronically in ASCII format and is hereby incorporated by reference in its entirety. Said ASCII copy, created on Apr. 27, 2021, is named 0076-0031US1_SL.txt and is 137,432 bytes in size.

### FIELD OF THE INVENTION

The invention relates to methods and kits for detecting a virus, e.g., a respiratory virus such as a coronavirus, in a biological sample. The invention also relates to methods and kits for detecting and/or quantifying biomarkers, e.g., antibody biomarkers against a viral antigen; or inflammatory and/or tissue damage response biomarkers in response to a viral infection.

### BACKGROUND

Respiratory viruses, including coronaviruses, can cause outbreaks of severe respiratory illnesses that place great burden on communities and healthcare systems. During an outbreak, large-scale tests are needed to identify infected but asymptomatic or mildly ill individuals, which can mitigate widespread disease transmission.

The COVID-19 pandemic created an urgent need for assays for multiple reasons, for example: to detect infection, to determine the stage of infection, e.g., viral load, to determine transmissibility of the virus, to determine presence or absence of virus, e.g., on surfaces, to aid in the development of vaccines, for epidemiological studies, to follow the immune status and past viral exposure of individuals, for research into factors contributing to morbidity and mortality of viral infection. Although some assays were developed early in the pandemic, they were slow or low throughput, lacked sensitivity, were inaccurate, were expensive, or otherwise inadequate. For example, current PCR-based tests, e.g., for SARS-CoV-2, are analytically sensitive but require a lengthy, complex, and expensive sample processing procedure, and may be difficult to run at the scale needed to screen large populations. Moreover, accurate and sensitive serology tests can be useful for epidemiological studies and to identify individuals who are immune or at low risk of infection. Thus, high-quality assays are desperately needed to address the pandemic.

### SUMMARY OF THE INVENTION

In embodiments, the invention provides an immunoassay method for detecting a coronavirus in a biological sample, comprising: (a) contacting the biological sample with a binding reagent that specifically binds a component of the coronavirus; (b) forming a binding complex comprising the binding reagent and the coronavirus component; and (c) detecting the binding complex, thereby detecting the coronavirus in the biological sample. In embodiments, the coronavirus component is a coronavirus nucleic acid. In embodiments, the coronavirus component is a coronavirus protein. In embodiments, the coronavirus is SARS-CoV-2.

In embodiments, the invention provides an immunoassay or nucleic acid detection method for detecting at least one respiratory virus in a biological sample, comprising: (a) contacting the biological sample with a binding reagent that specifically binds a component of at least one respiratory virus in the biological sample; (b) forming a binding complex comprising the binding reagent and the respiratory virus component; and (c) detecting the binding complex, thereby detecting the at least one respiratory virus in the biological sample. In embodiments, the method is a multiplexed immunoassay method. In embodiments, the respiratory virus is a coronavirus. In embodiments, the respiratory virus is SARS-CoV-2.

In embodiments, the invention provides an immunoassay method comprising: quantifying the amounts of one or more respiratory virus antigens and/or or one or more biomarkers capable of binding to a respiratory virus antigen in a biological sample, wherein the respiratory virus is a coronavirus, an influenza virus, a paramyxovirus, an adenovirus, a bocavirus, a pneumovirus, an enterovirus, a rhinovirus, or a combination thereof, wherein the quantifying comprises measuring the concentrations of each of the one or more antigens and/or biomarkers in an immunoassay. In embodiments, the one or more biomarkers is a host biomarker. In embodiments, the method is a multiplexed immunoassay. In embodiments, the one or more biomarkers is an antibody biomarker. In embodiments, the immunoassay is a bridging serology assay. In embodiments, the immunoassay is a classical serology assay. In embodiments, the immunoassay is a competitive immunoassay. In embodiments, the respiratory virus is a coronavirus. In embodiments, the respiratory virus is SARS-CoV-2.

In embodiments, the invention provides a multiplexed immunoassay method comprising: quantifying the amounts of one or more viral antigens and/or biomarkers capable of binding to a viral antigen thereof in a biological sample, wherein the viral antigen comprises a spike protein (S), spike protein subunit 1 (S1), spike protein subunit 2 (S2), membrane protein (M; sometimes also called the matrix protein), envelope protein (E), nucleocapsid protein (N), or a variant or subunit, domain, or fragment thereof, or any combination thereof, wherein the quantifying comprises measuring the concentrations of each of the one or more antigens and/or biomarkers in an immunoassay. In embodiments, the one or more biomarkers is a host biomarker.

In embodiments, the invention provides an immunoassay method comprising: quantifying the amounts of one or more biomarkers in a biological sample, wherein the one or more biomarkers comprises C-reactive protein (CRP), IFNα2, IFN-γ, IL-6, IL-10, MCP-1, IP-10, troponin, skeletal troponin-I (sTnI), IL-1β, IL-2, IL-4, IL-7, granulocyte colony-stimulating factor (G-CSF). MIP-1α, TNF-α, ferritin, CD147, neurofilament light (NfL), kidney injury molecule-1 (KIM-1), IL-8, MIP-10, MCP-4, thymus and activation regulated chemokine (TARC, also known as CCL17), vascular endothelial growth factor receptor-1 (VEGFR-1, also known as Flt-1), phosphatidylinositol-glycan biosynthesis class F protein (PlGF). vascular endothelial growth factor A (VEGF-A), vascular endothelial growth factor C (VEGF-C), intercellular adhesion molecule 1 (ICAM-1), serum amyloid A (SAA), vascular cell adhesion protein 1 (VCAM-1), angiopoietin-2 (Ang-2), Lectin Binding Protein (LBP), an inflammatory damage biomarker, a tissue damage biomarker, a T cell activation biomarker, an inflammatory response biomarker, a tissue damage biomarker, an extracellular vesicle, or any combination thereof, wherein the quantifying comprises measuring the concentrations of each of the one or more biomarkers in an immunoassay. In embodiments, the one or more biomarkers is a host biomarker. In embodiments, the method is a multiplexed immunoassay.

In embodiments, the invention provides a kit comprising, in one or more vials, containers, or compartments: (a) a binding reagent that specifically binds a respiratory virus component; and (b) a detection reagent that specifically binds the respiratory virus component. In embodiments, the kit comprises a surface.

In embodiments, the invention provides a kit comprising, in one or more vials, containers, or compartments: (a) a viral antigen that specifically binds a biomarker; and (b) a detection reagent that specifically binds the biomarker. In embodiments, the kit comprises a surface. In embodiments, the invention provides a kit comprising, in one or more vials, containers, or compartments: (a) a binding reagent that specifically binds a biomarker; and (b) a detection reagent that specifically binds the biomarker. In embodiments, the kit comprises a surface.

### BRIEF DESCRIPTION OF THE DRAWINGS

The following drawings form part of the present specification and are included to further demonstrate exemplary embodiments of certain aspects of the present invention.

FIG. 1 relates to Example 4. FIG. 1 shows the results of an embodiment of a serology assay described herein. A panel of viral antigens were immobilized as binding reagents, and anti-IgG or anti-IgM antibodies were used as detection reagents. The serology assay was tested on serum samples from COVID-19 positive (red circles) and normal (non-COVID-19) (blue circles) patients, diluted 500-fold. Higher signal indicates increased number of antibodies bound to the immobilized antigen.

FIG. 2 relates to Example 5A. FIG. 2 shows the results of an embodiment of a bridging serology assay described herein. SARS-CoV-2 S-RBD was immobilized as binding reagent, and labeled S-RBD was used as detection reagent. The bridging serology assay was tested on serum samples from COVID-19 positive (red circles) and normal (non-COVID-19) (blue circles) patients, diluted 10-fold or 100-fold. Higher signal indicates increased number of antibodies bound to the immobilized antigen.

FIG. 3 relates to Example 6A. FIG. 3 shows the results of an embodiment of a neutralization serology assay described herein. SARS-CoV-2 S protein was immobilized as binding reagent, and labeled ACE2 was added as a competitor to SARS-CoV-2 antibodies that may be present. The neutralization serology assay was tested on serum samples from COVID-19 positive (red circles) and normal (non-COVID-19) (blue circles) patients, diluted 10-fold or 100-fold. Lower signal (generated by competitor) indicates increased number of antibodies bound to the immobilized antigen.

FIGS. 4 and 5 relate to Example 7. FIG. 4 shows the results of embodiments of SARS-CoV-2 detection assays described herein, utilizing a binding reagent that specifically binds SARS-CoV-2 nucleocapsid (N) protein. The blue curve presents the calibration curve for an assay utilizing a detection reagent comprising a detectable label. The red curve presents the calibration curve of an assay utilizing a detection reagent comprising a nucleic acid probe. The assays were tested on recombinant sample containing known concentrations of the SARS-CoV-2 N protein. The graph shows the best 4 parameter logic (4PL) fits to the data. The limit of detection (the concentration that provides a signal 2.5 standard deviations above background, calculated using the 4PL fit) is shown as a vertical dashed line.

FIG. 5 shows the assay signal as a function of virus concentration, presented as genome equivalents per mL, for a radiation-inactivated SARS-CoV-2 viral preparation determined based on the calibration curves shown in FIG. 4. The graph shows the best 4 parameter logic (4PL) fits to the data. The limit of detection (the concentration that provides a signal 2.5 standard deviations above background, calculated using the 4PL fit) is shown as a vertical dashed line.

FIG. 6 relates to Example 8. FIG. 6 shows the results of an embodiment of a SARS-CoV-2 detection assay described herein, utilizing a binding reagent that specifically binds SARS-CoV-2 nucleocapsid (N) protein and a detection reagent comprising a nucleic acid probe. The detection assay was tested on extracted swab samples from commercial vendors that included 10 putatively negative samples (blue) and 20 putatively positive samples (red).

FIG. 7 illustrates an embodiment of the methods described herein for detecting a viral nucleic acid. An oligonucleotide binding reagent comprises, in 5′ to 3′ order, a targeting agent complement, an amplification primer, a target hybridization region, an amplification blocker, and a secondary targeting agent complement (TAC). The binding reagent hybridizes with the analyte nucleic acid, e.g., coronavirus nucleic acid to form a binding complex. The binding complex is contacted with a Cas nickase, which nicks the binding reagent to remove the secondary TAC and amplification blocker, thereby activating the amplification primer for an amplification cycle. After one or more amplification cycles, the reaction mixture sample is incubated on a secondary surface comprising a secondary targeting agent, which removes any cleaved amplification blocker-secondary targeting agent complement and uncleaved binding reagent. Following the incubation on the secondary surface, the reaction mixture sample is then incubated on a surface comprising a targeting agent to immobilize the binding complex(es) onto the surface. The immobilized binding complex(es) is then subjected to extension and detection as described herein.

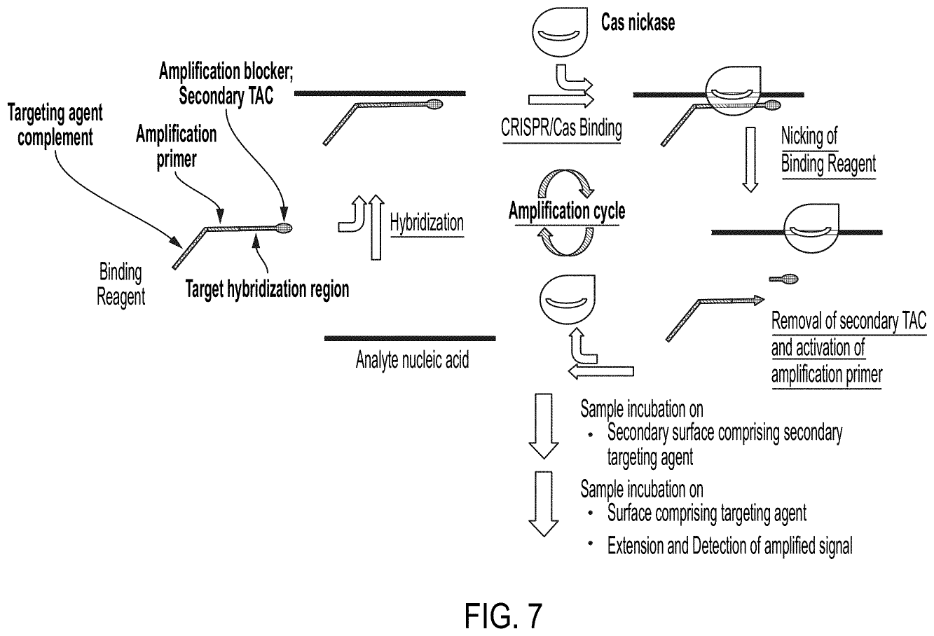

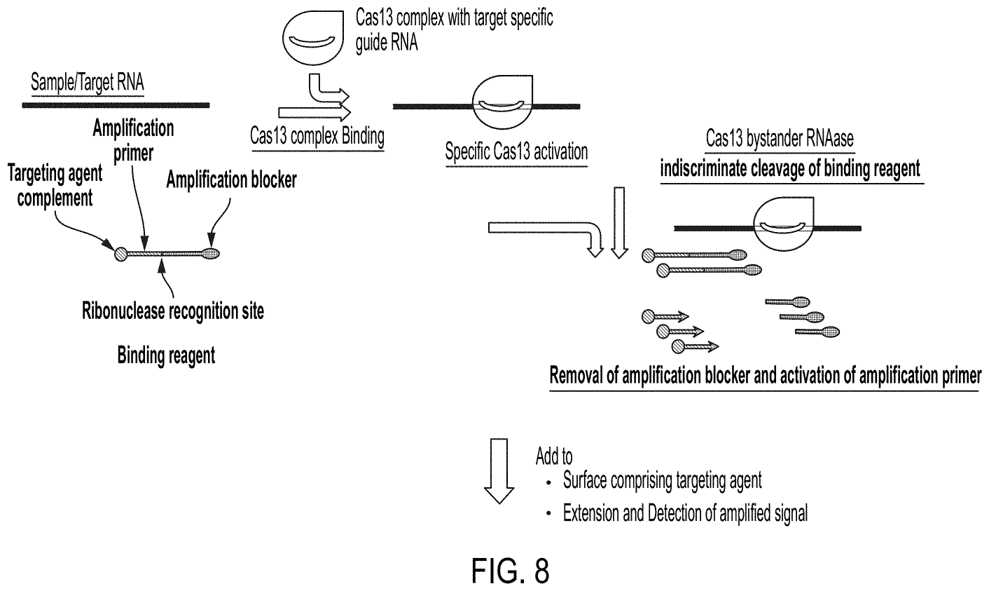

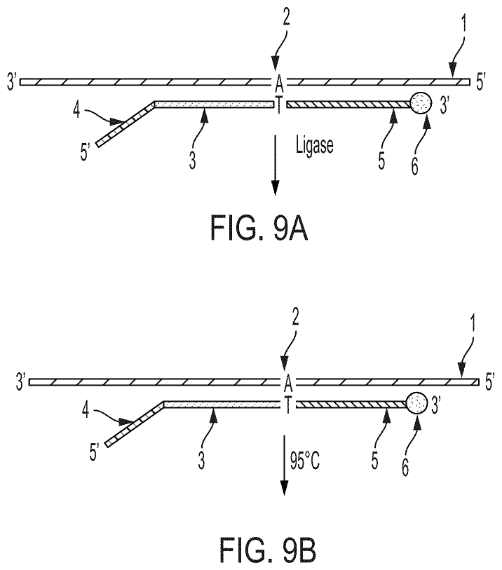

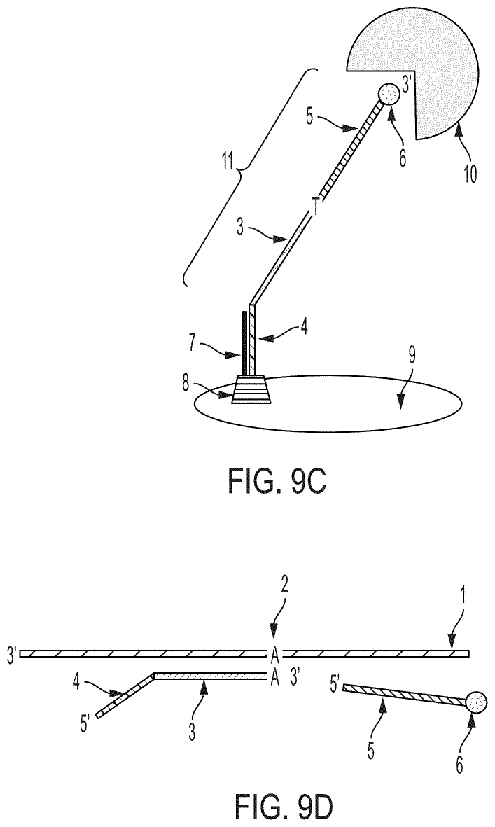

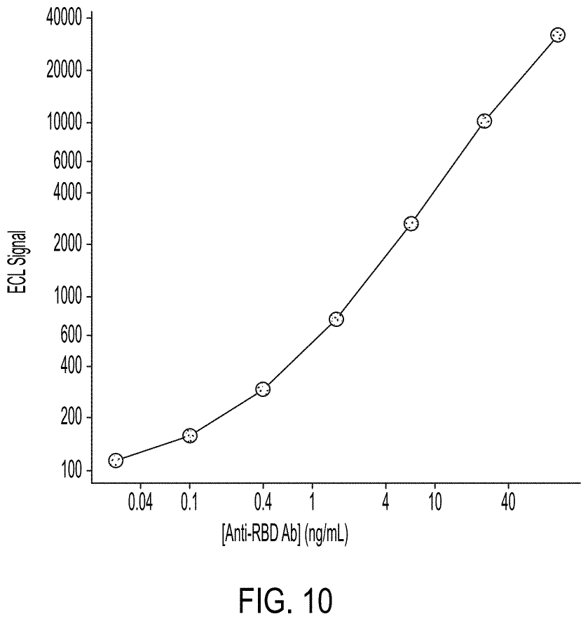

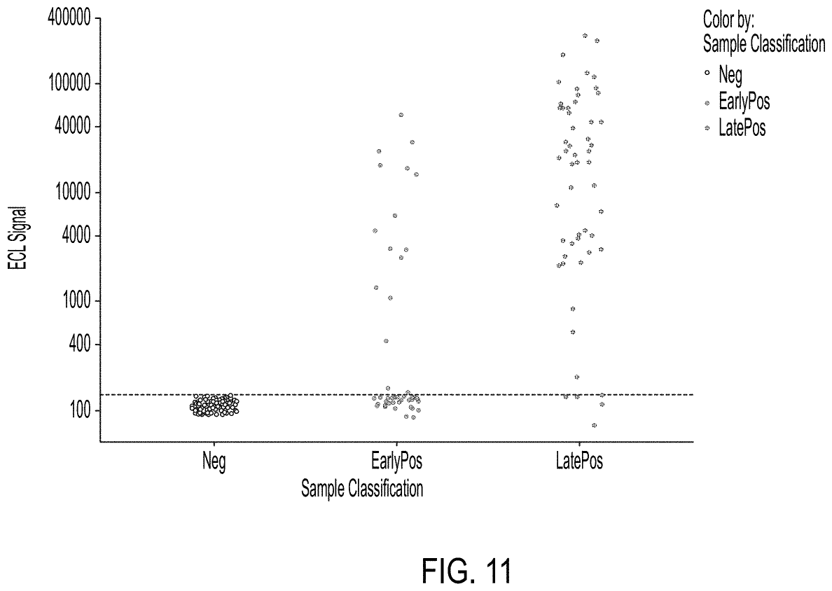

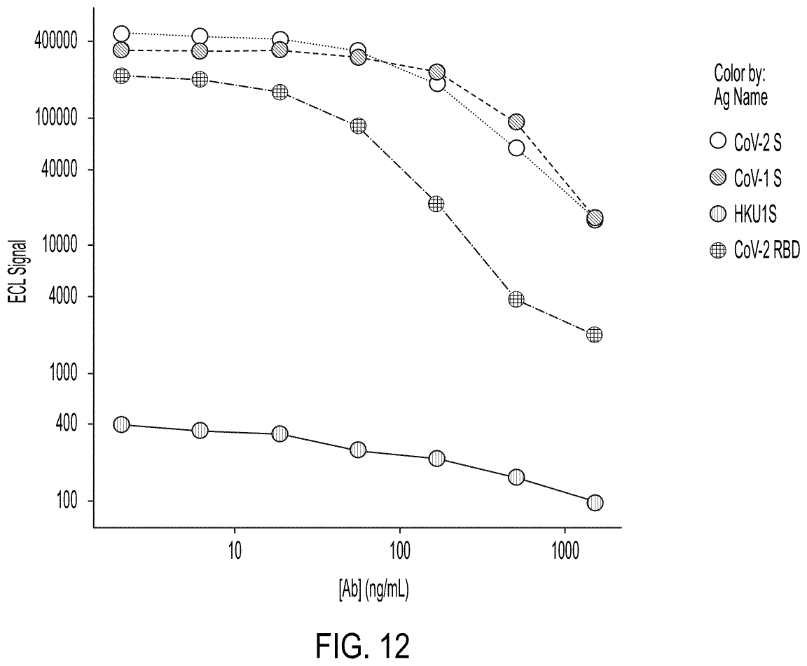

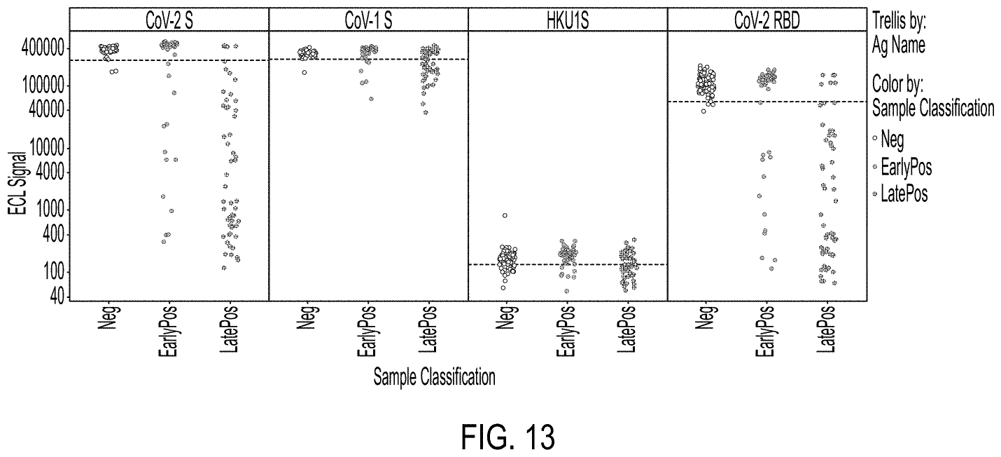

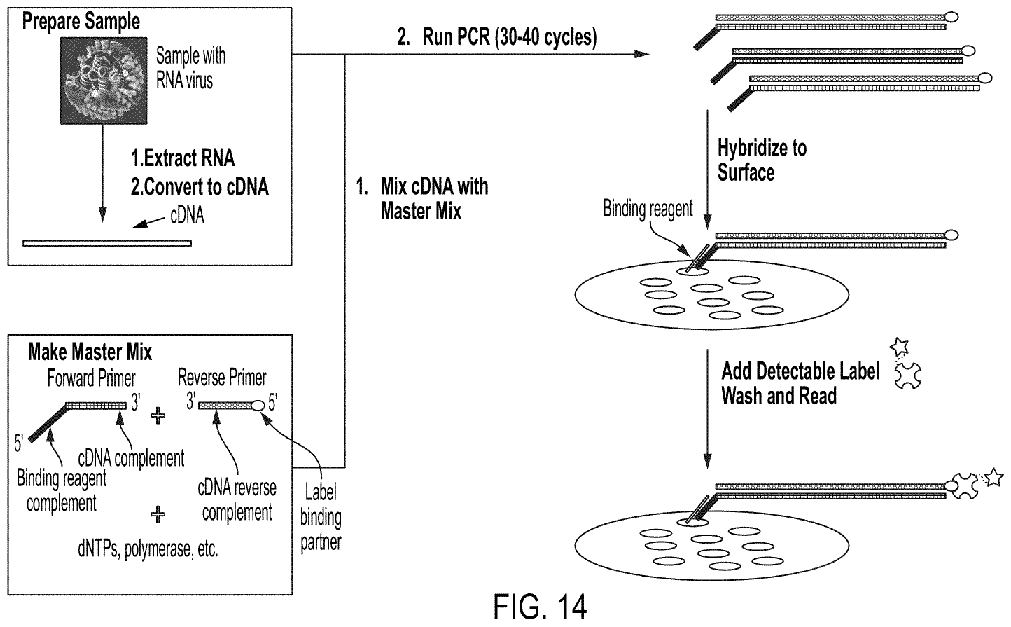

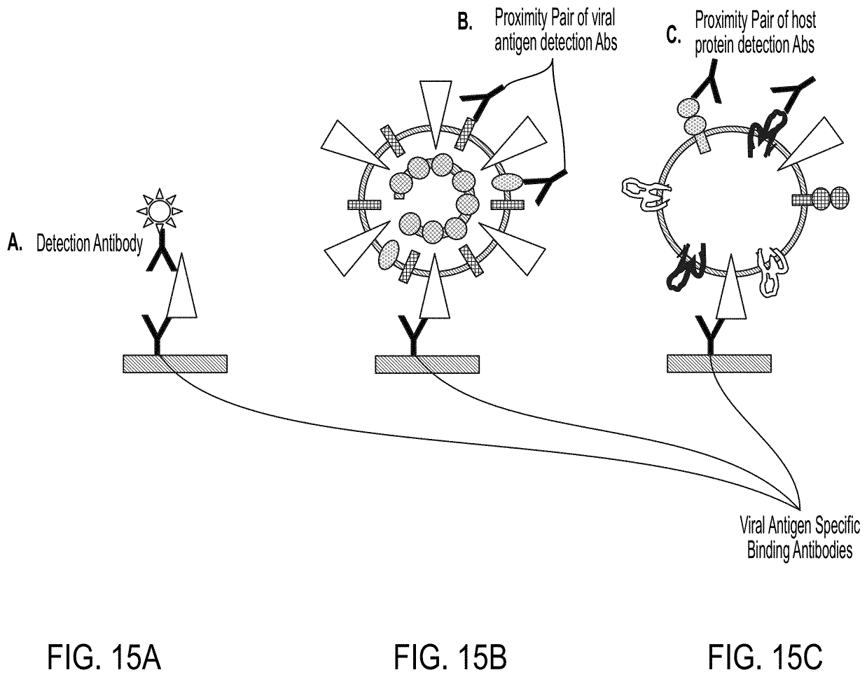

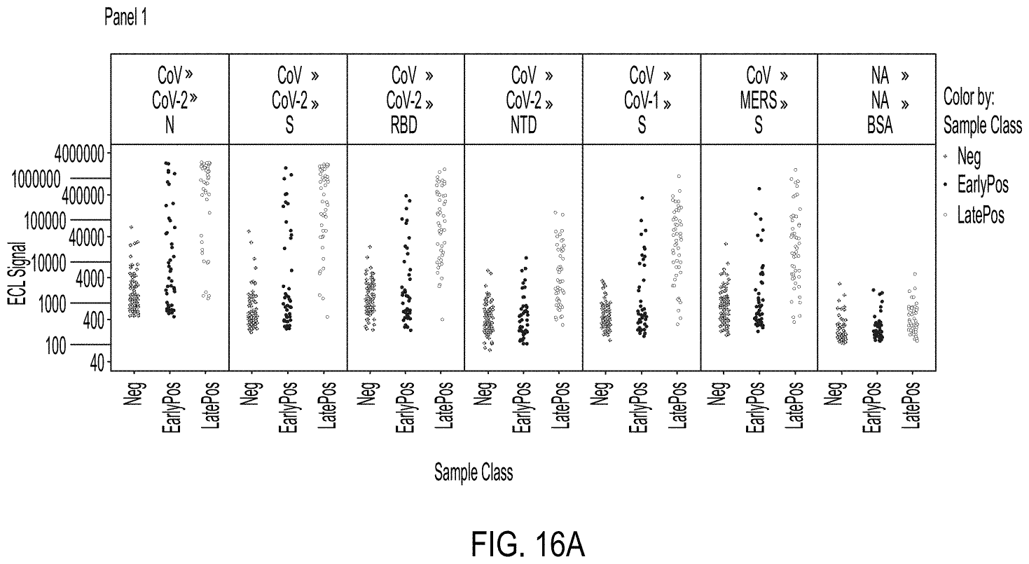

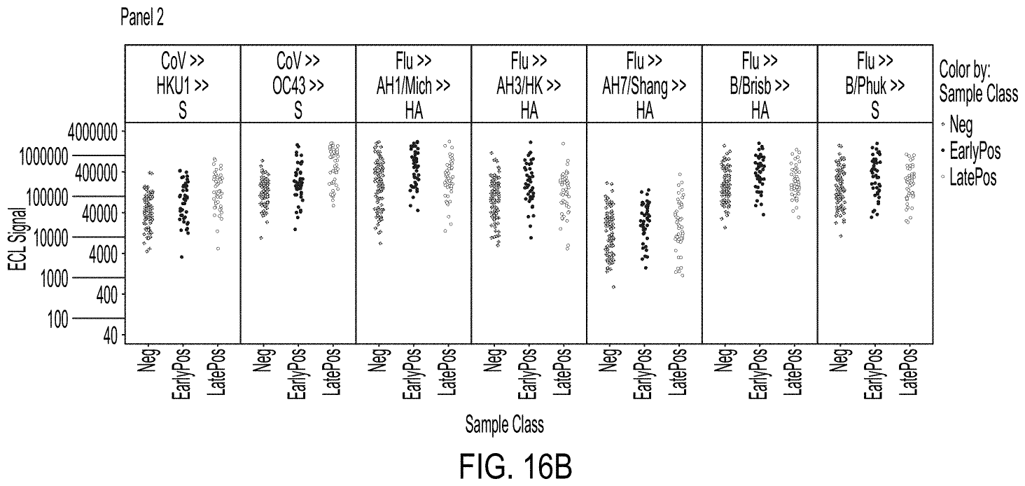

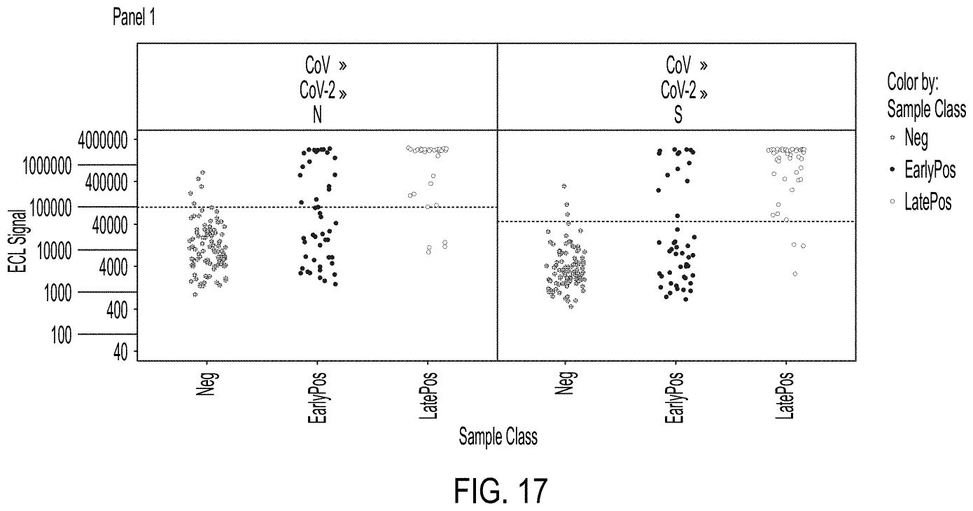

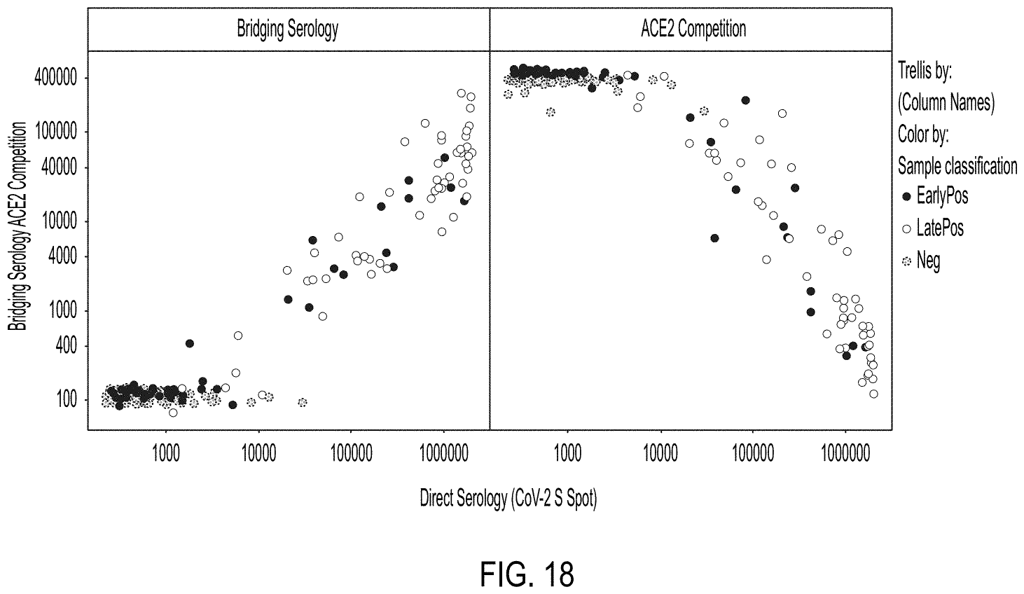

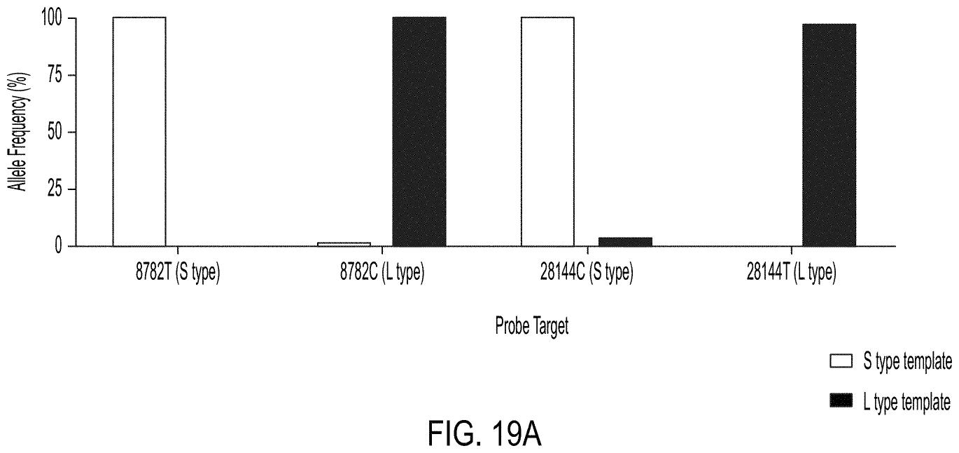

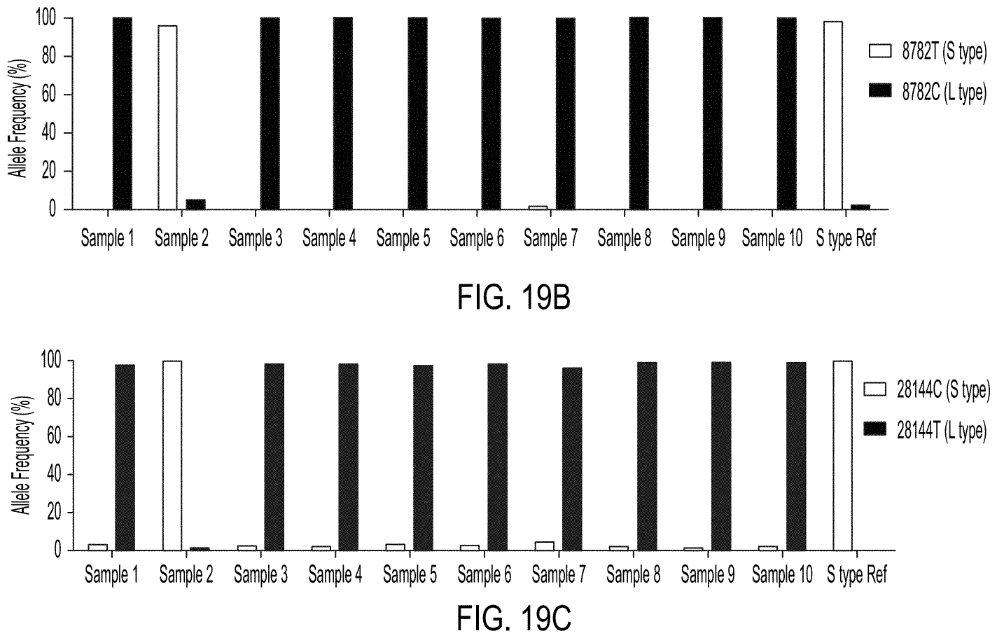

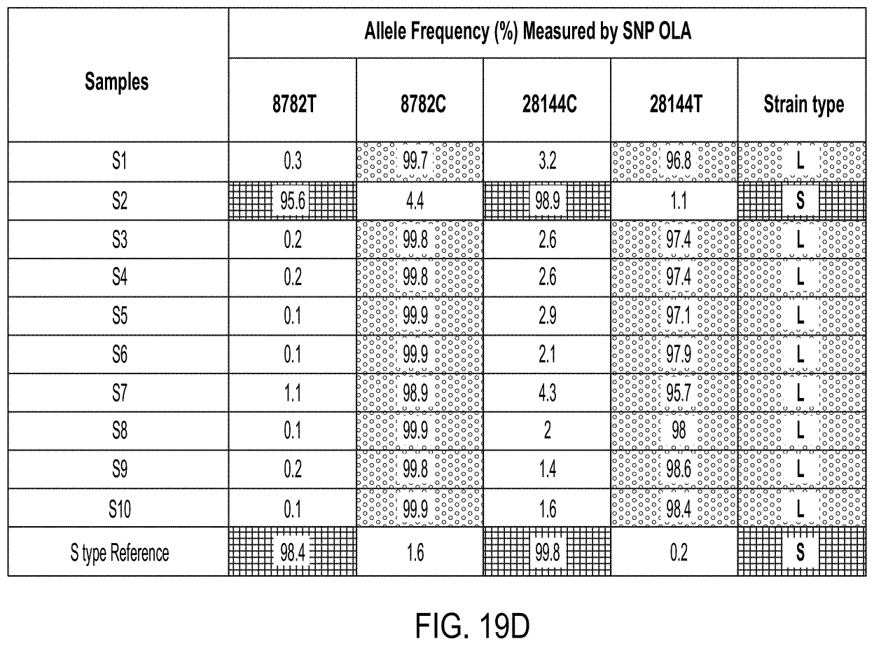

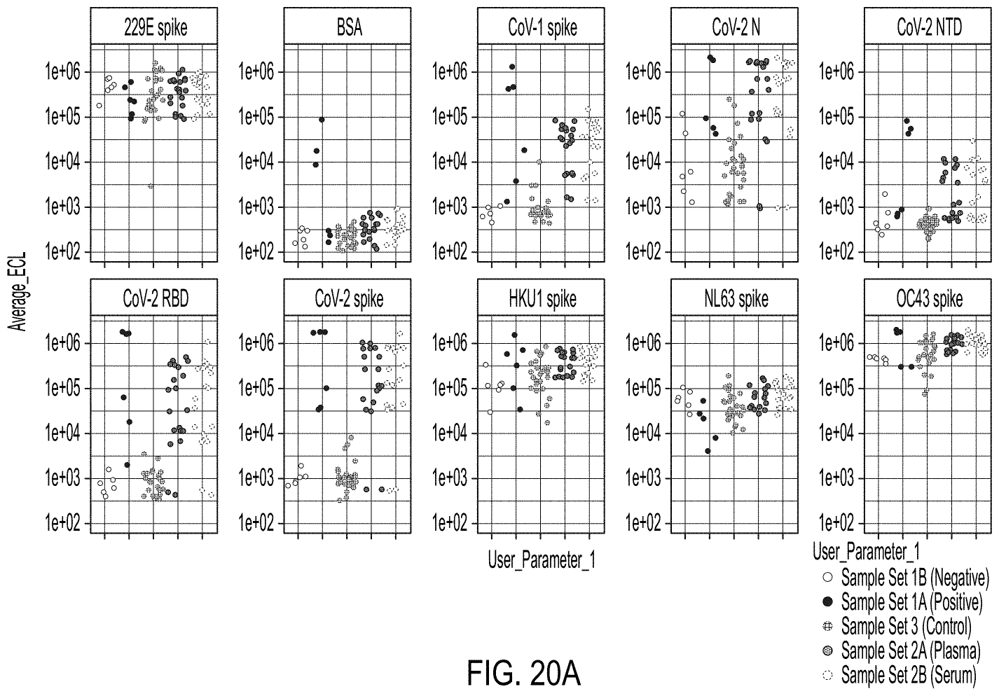

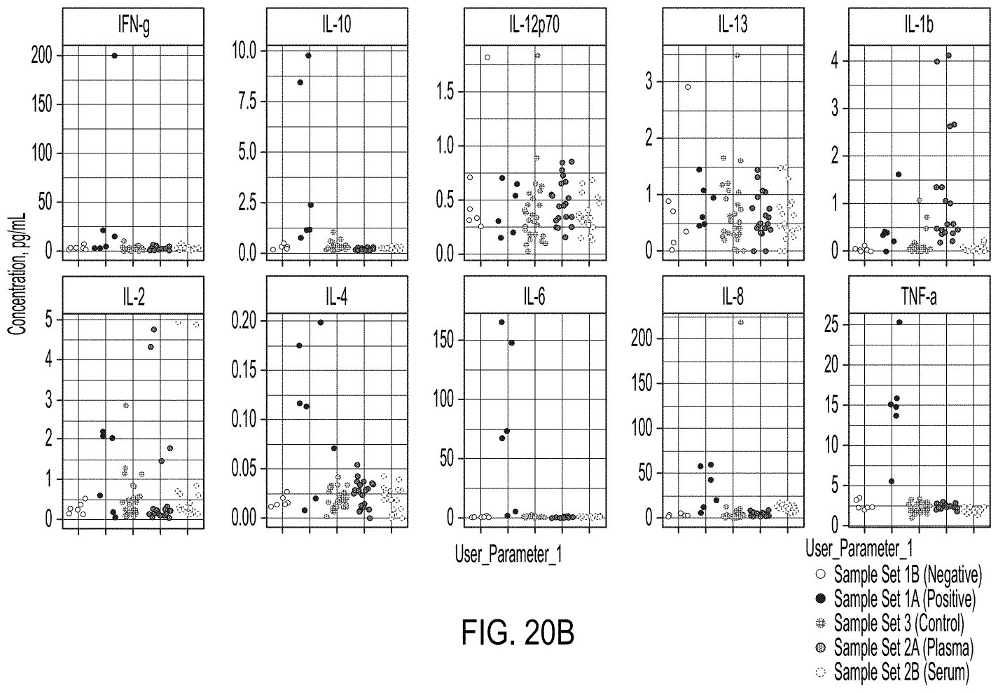

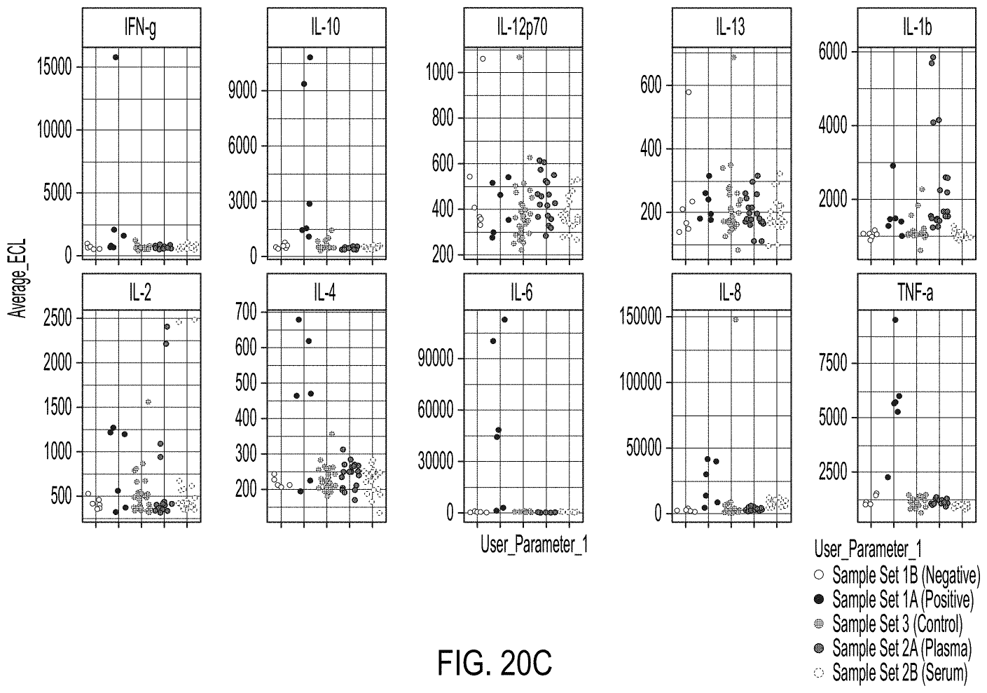

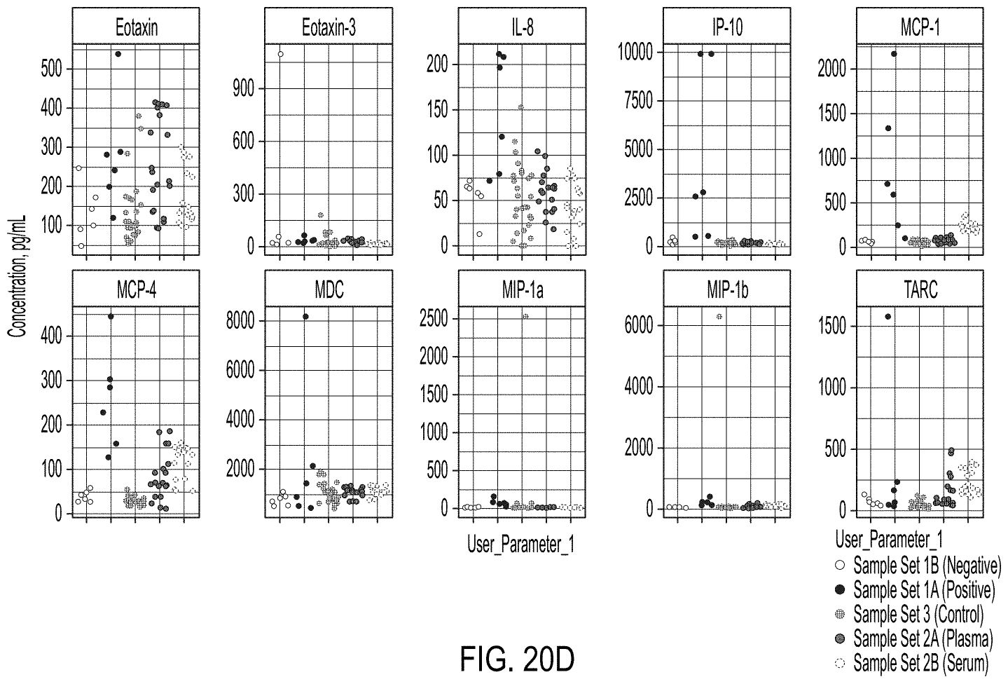

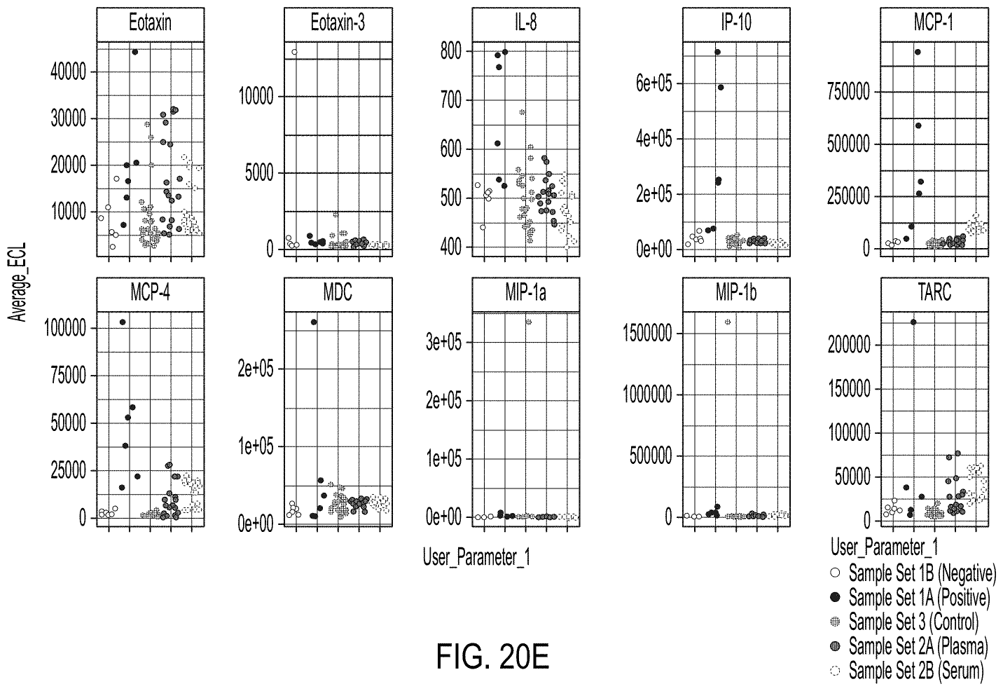

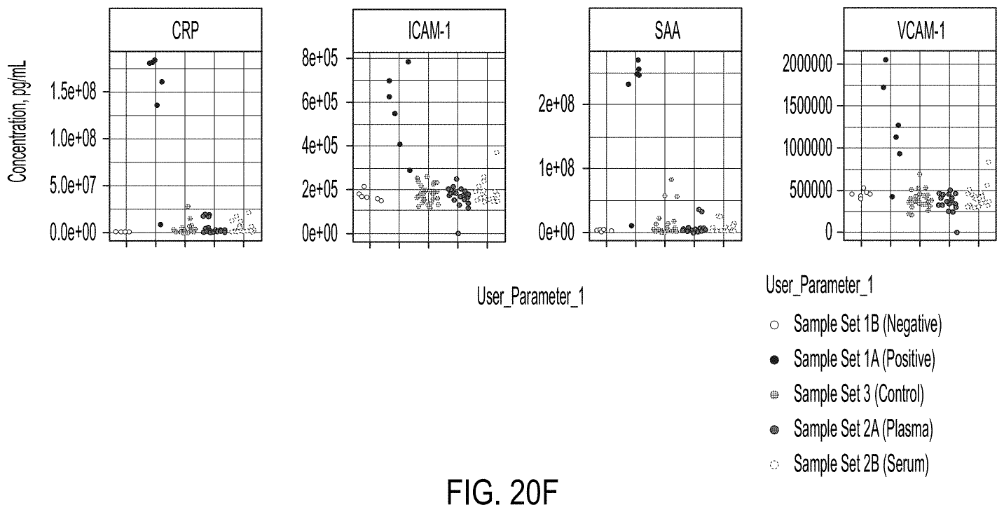

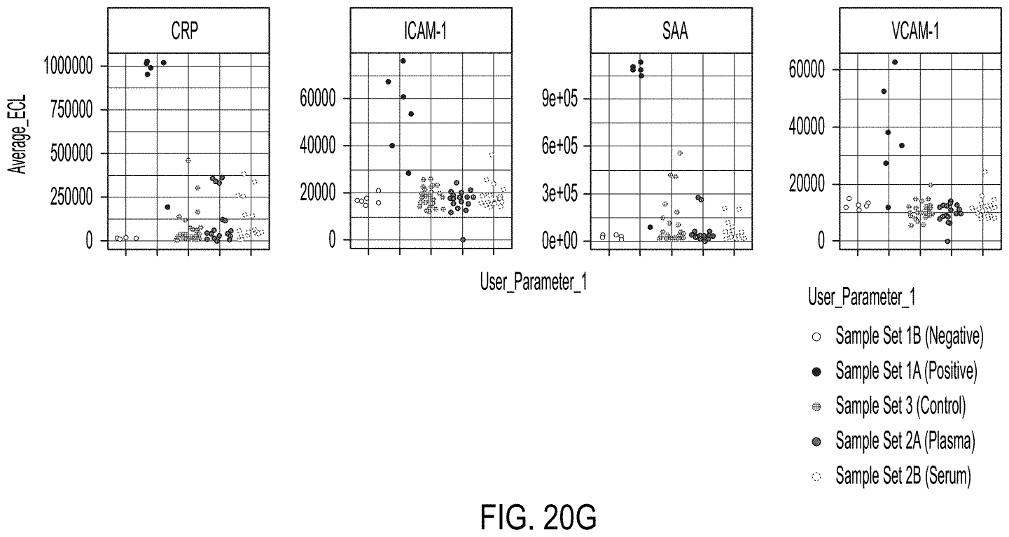

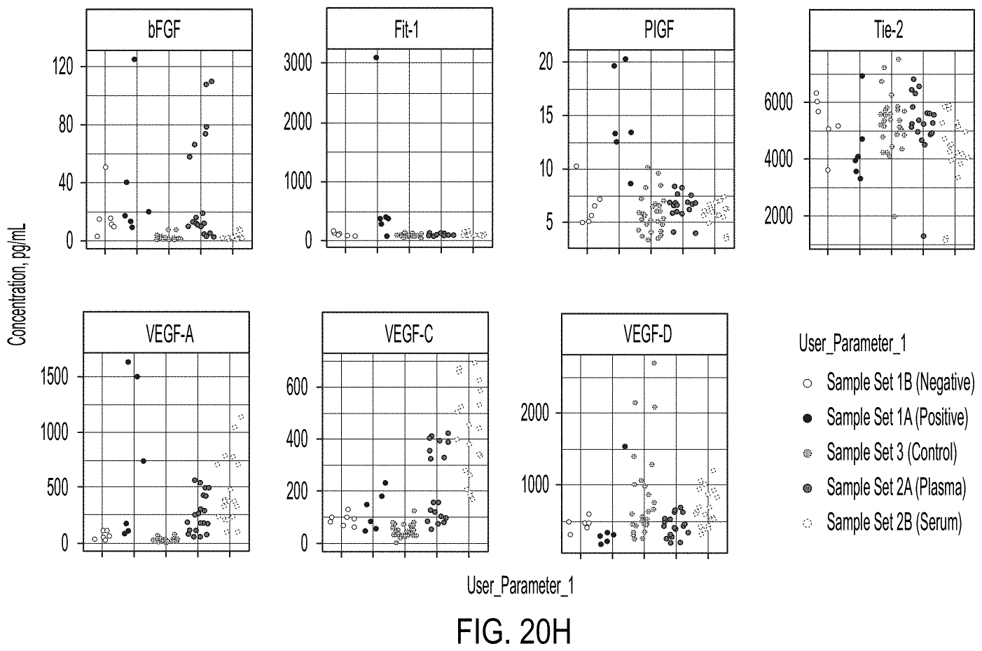

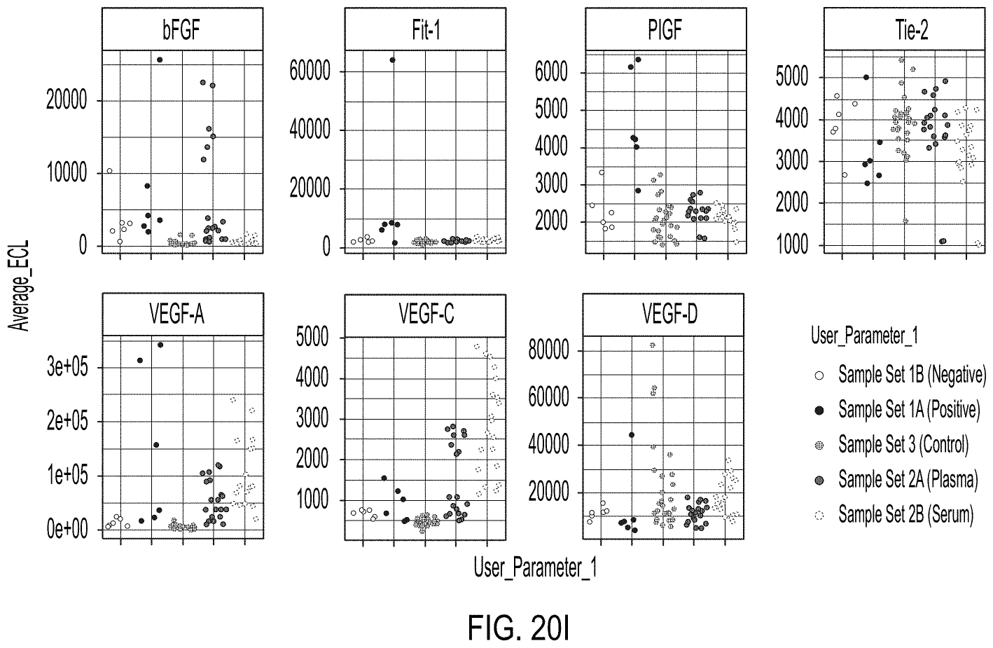

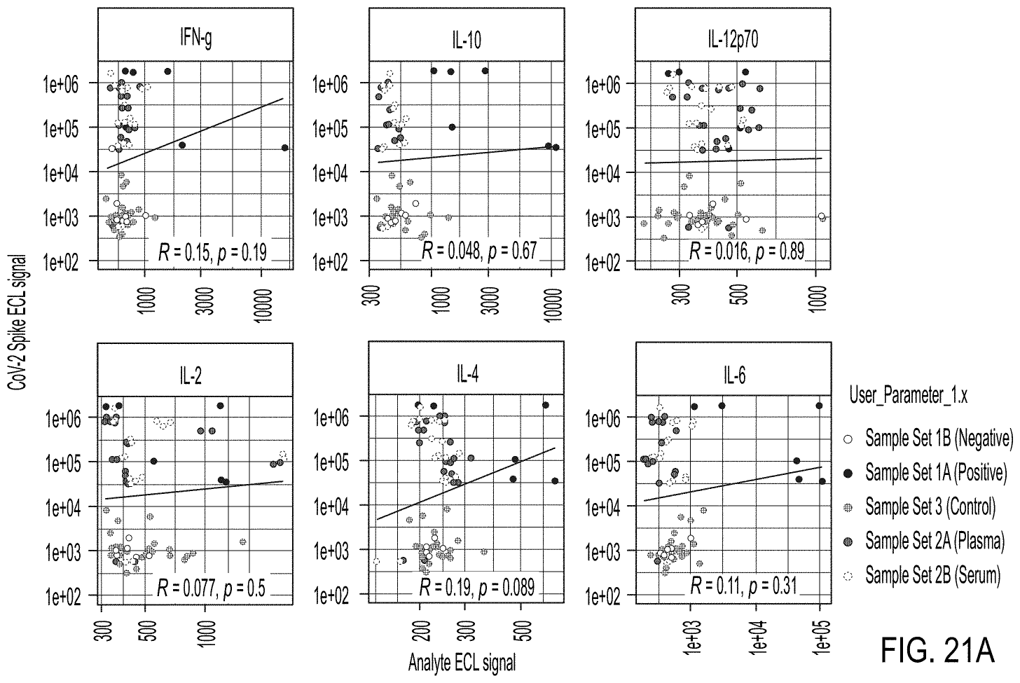

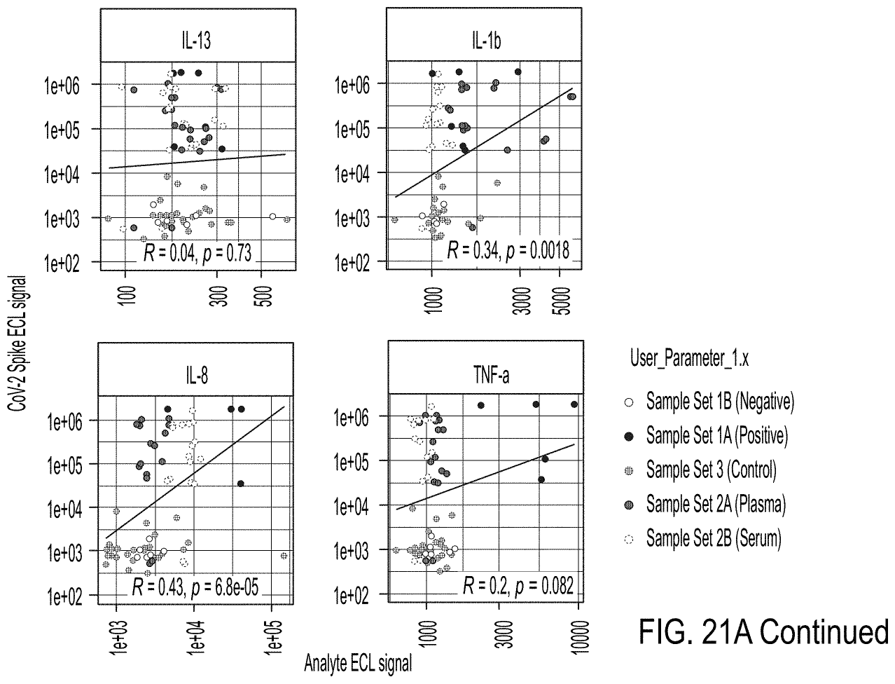

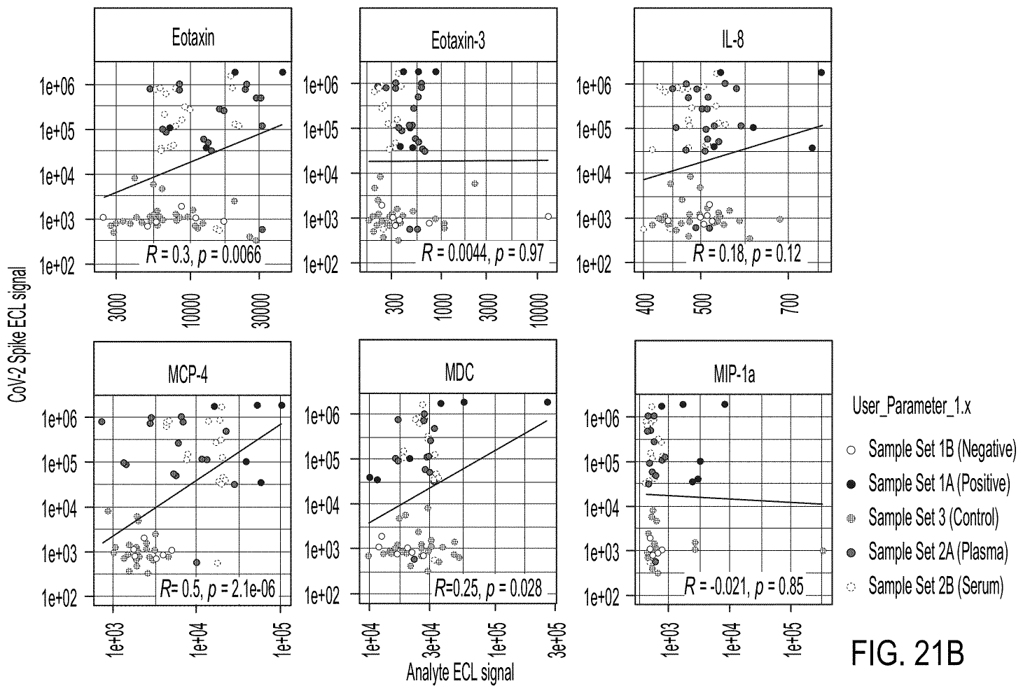

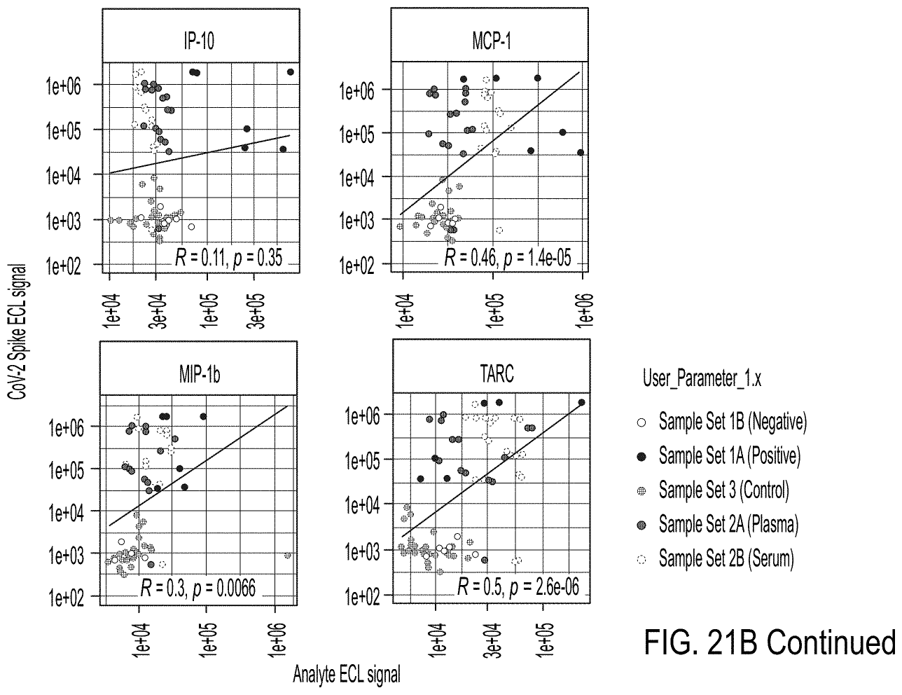

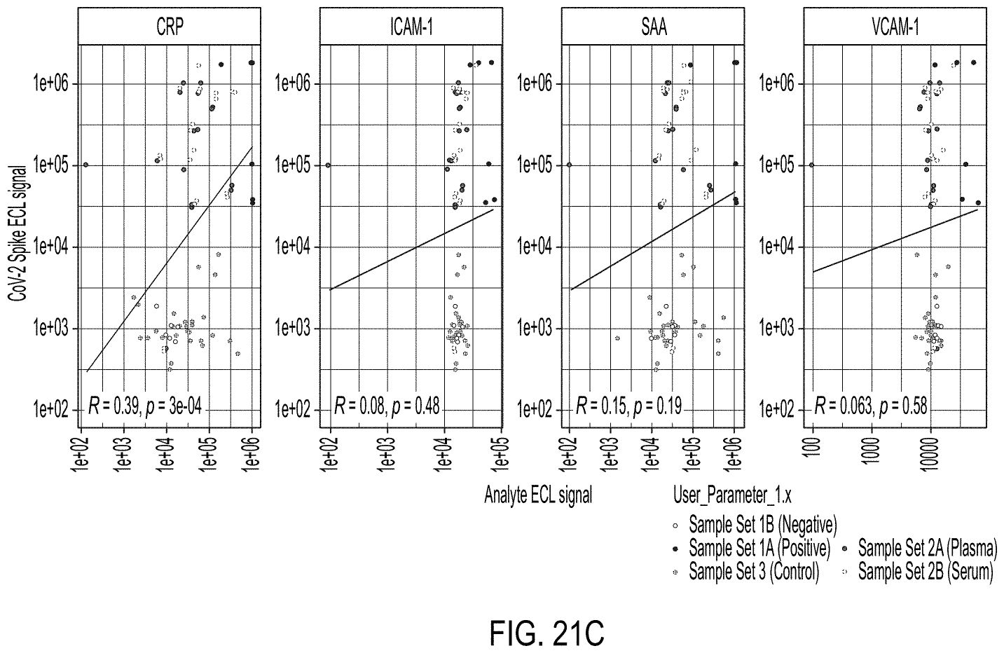

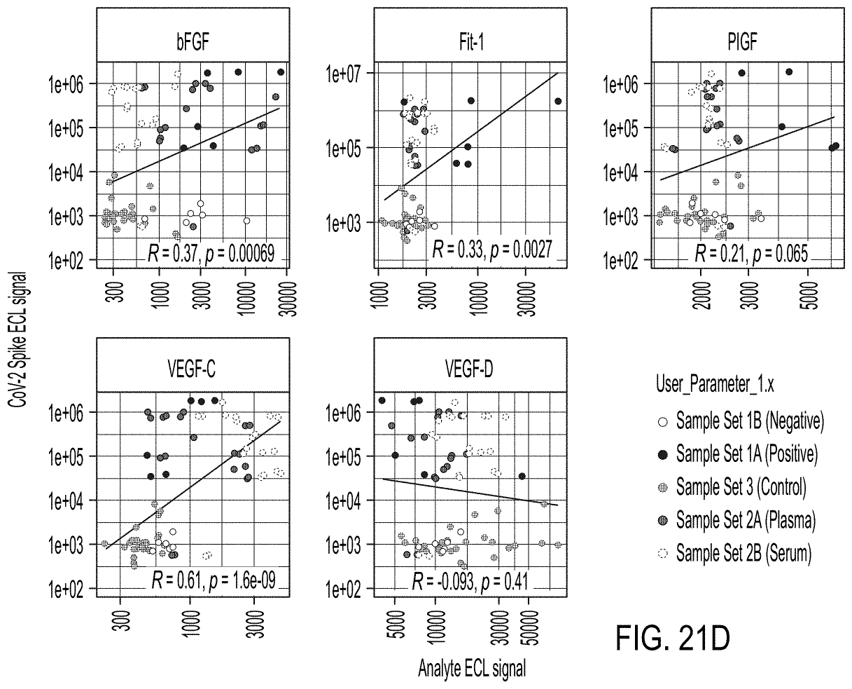

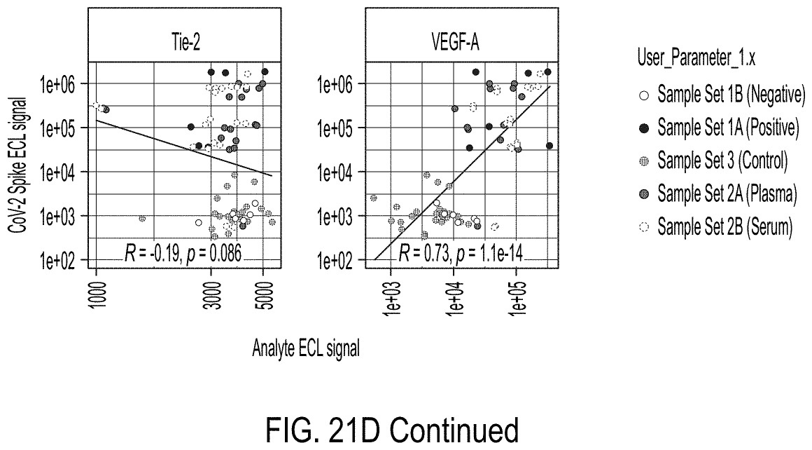

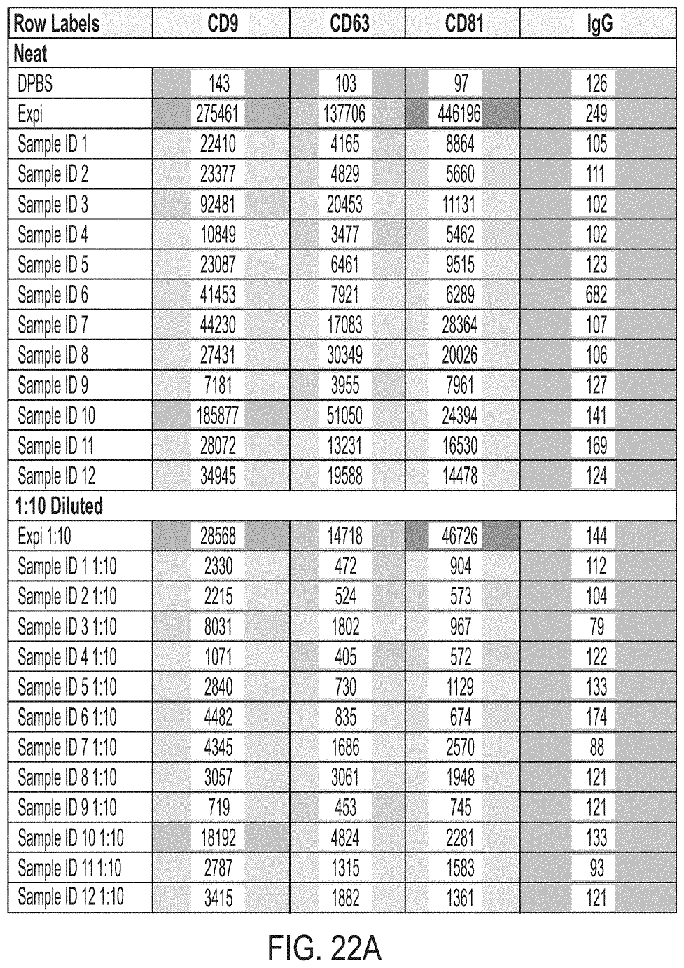

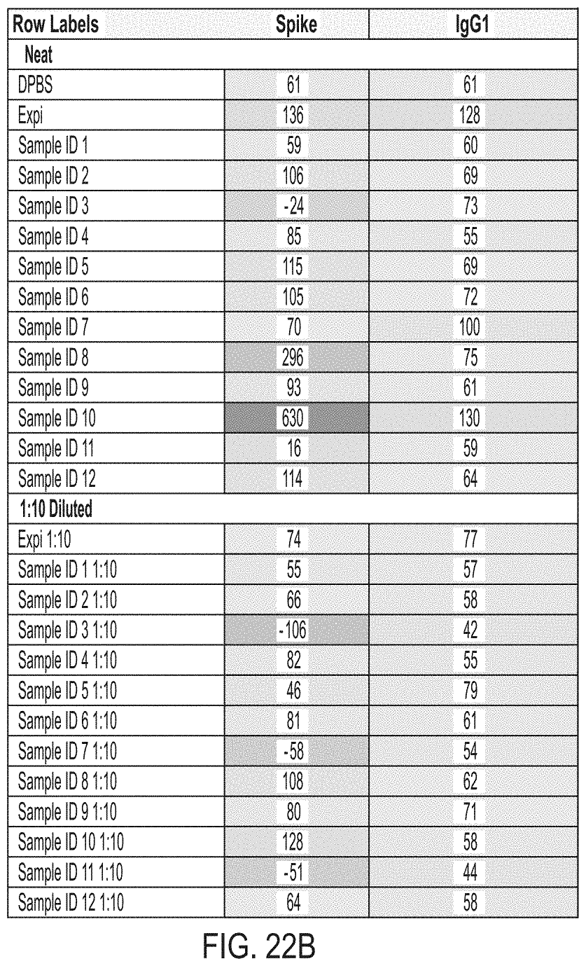

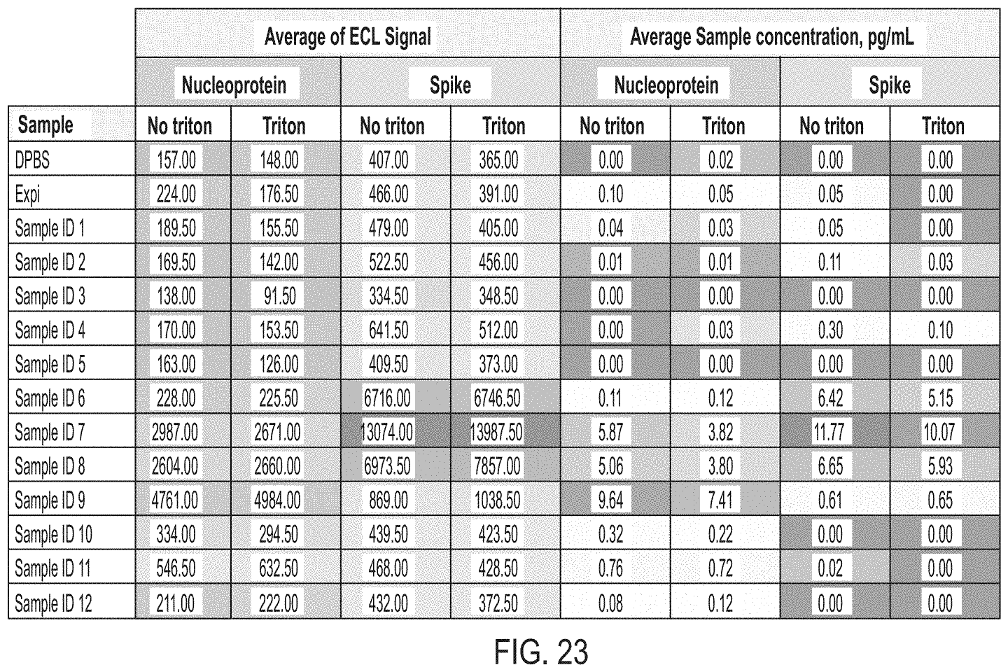

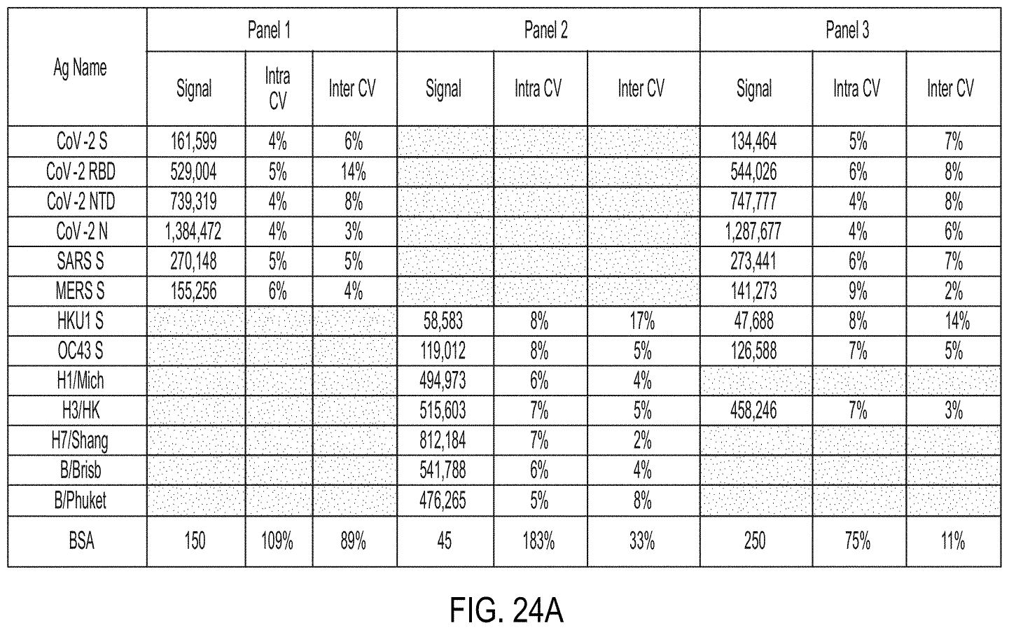

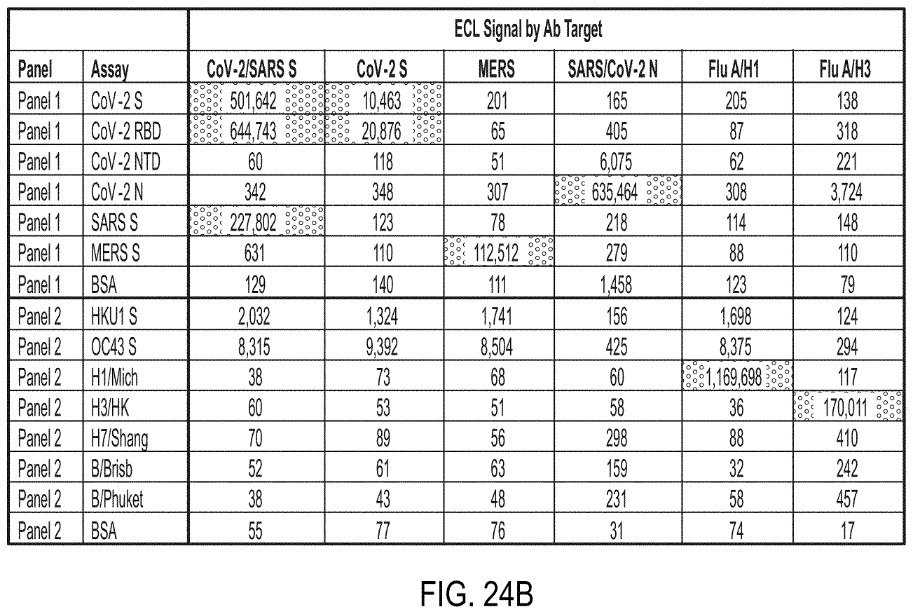

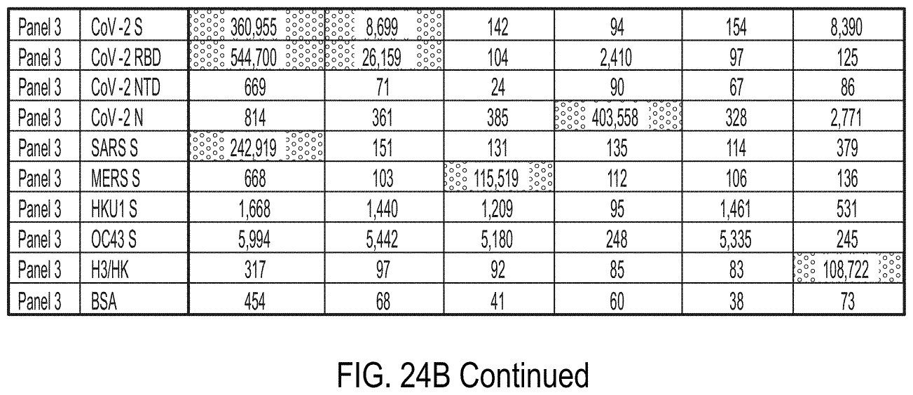

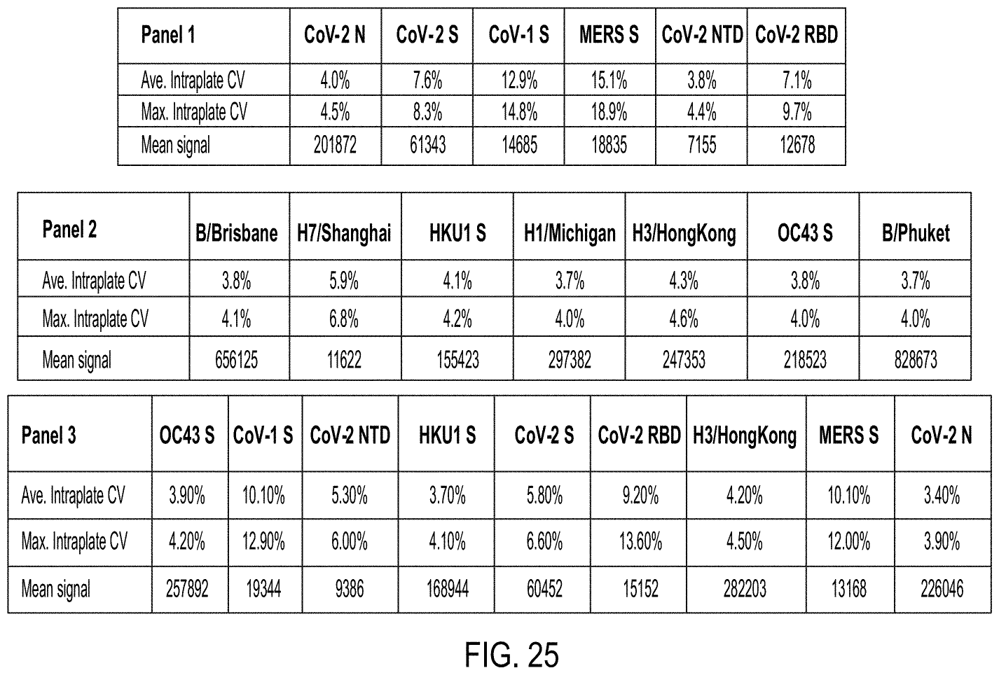

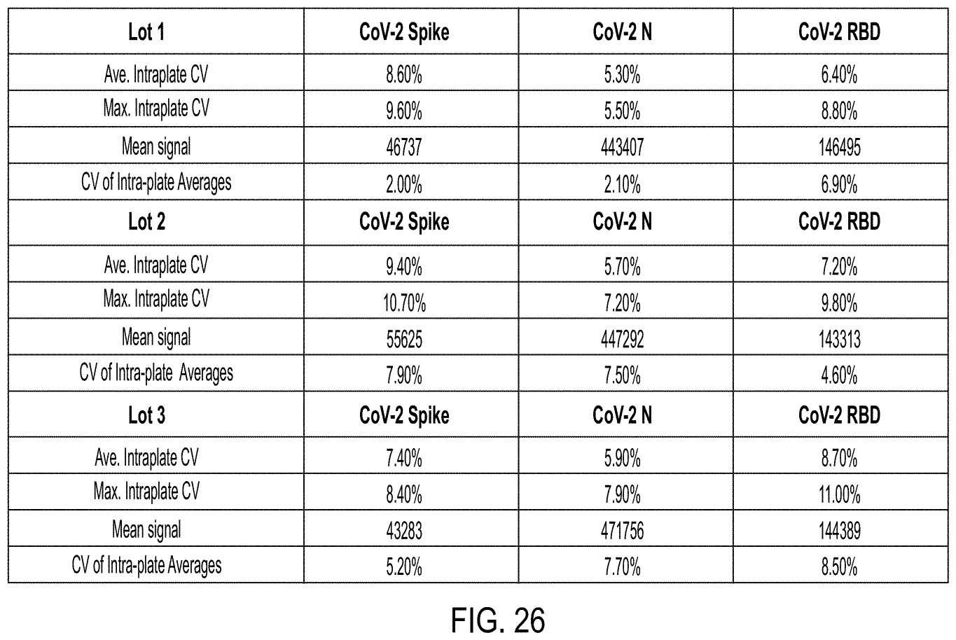

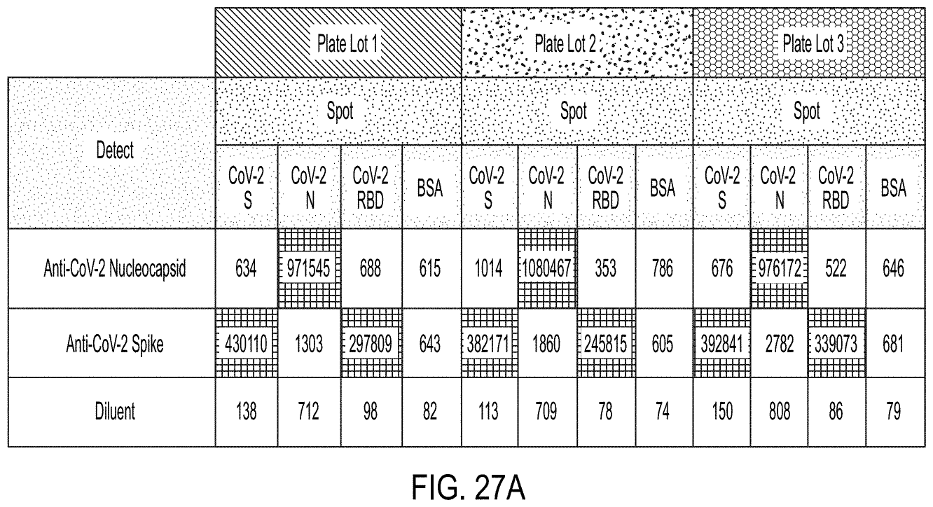

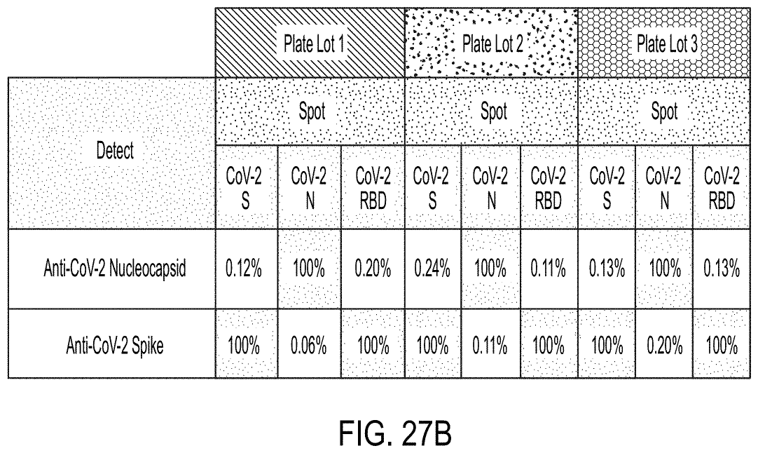

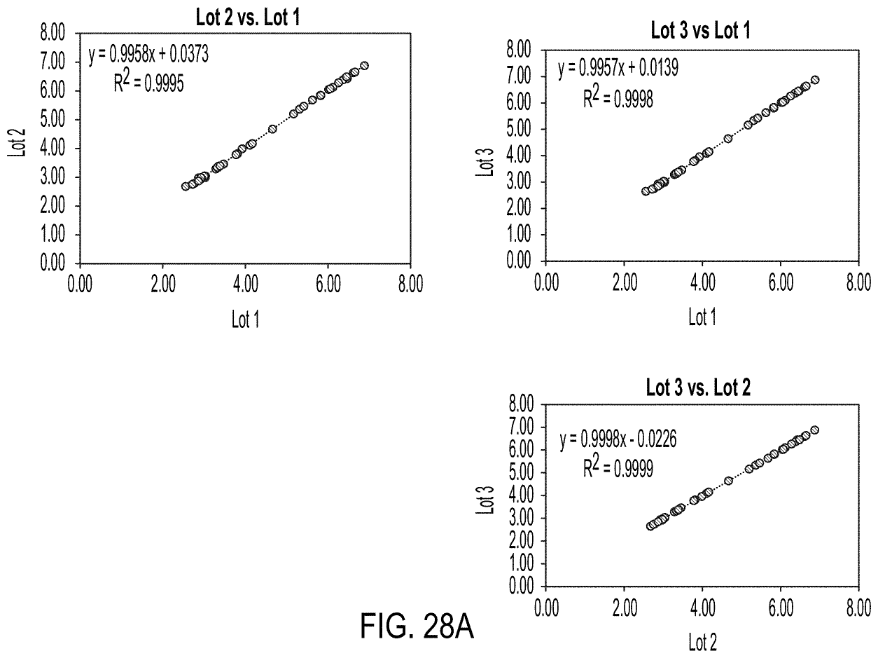

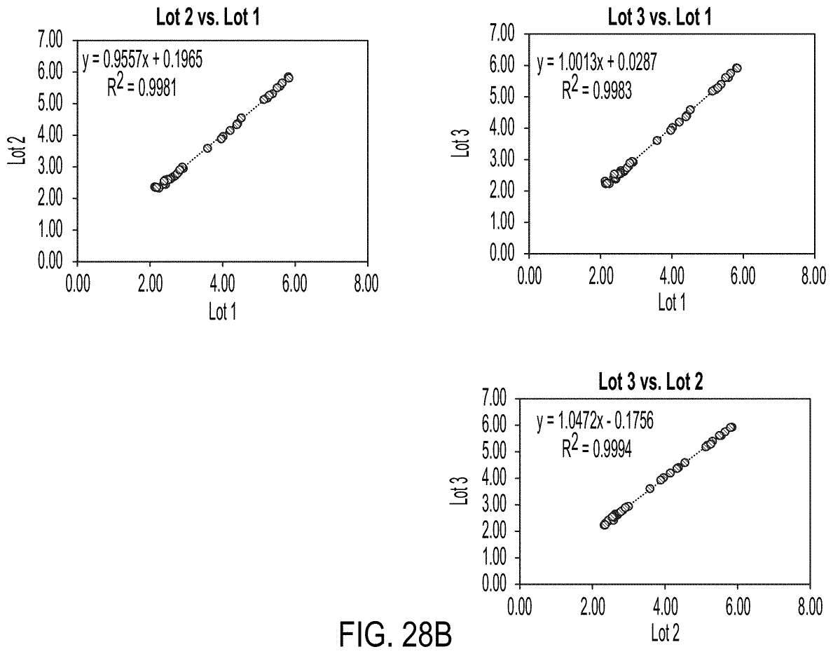

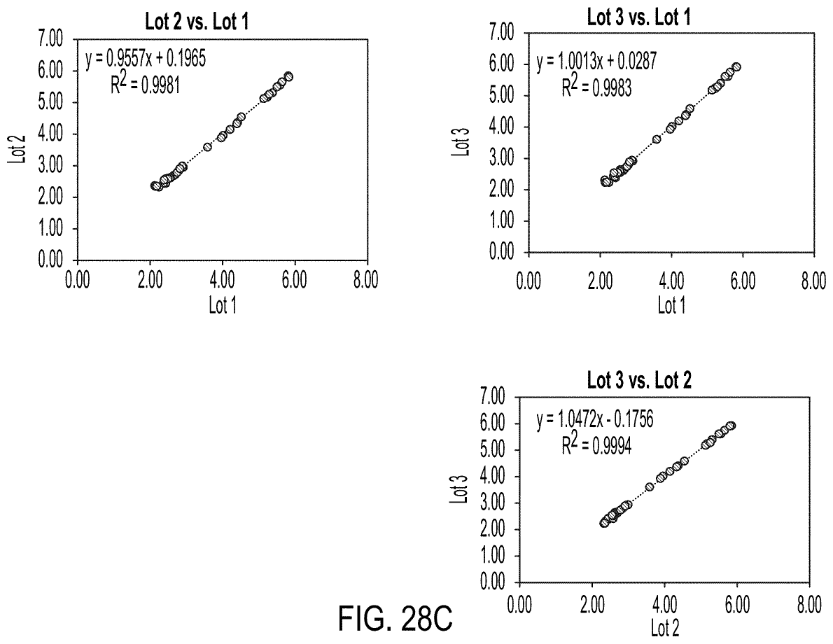

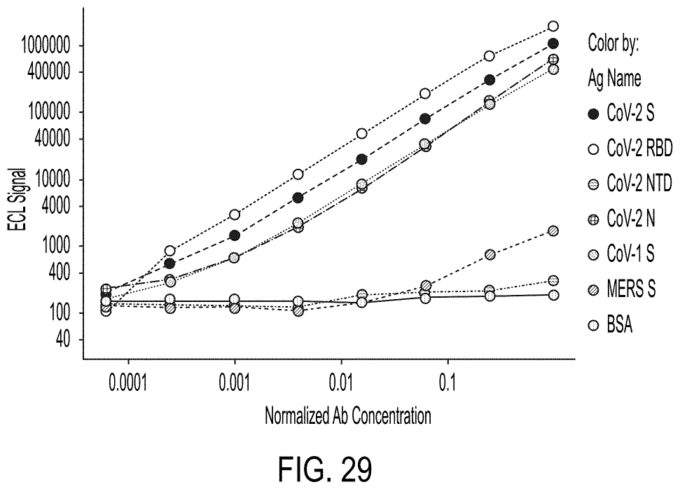

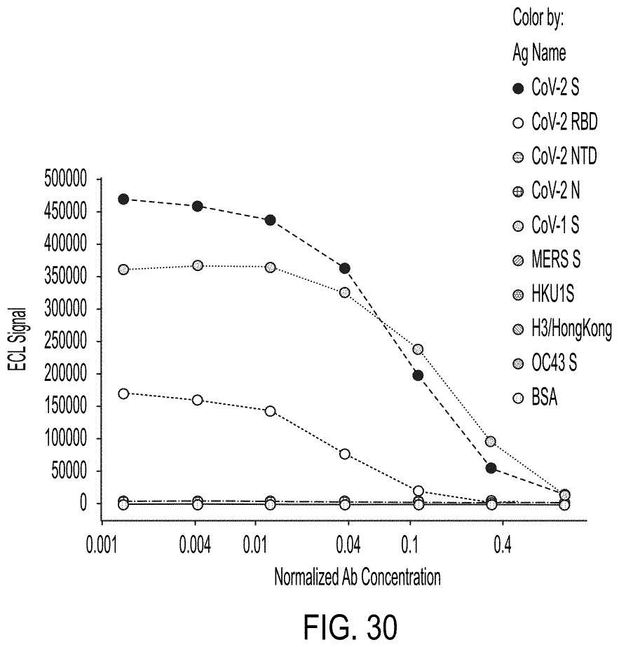

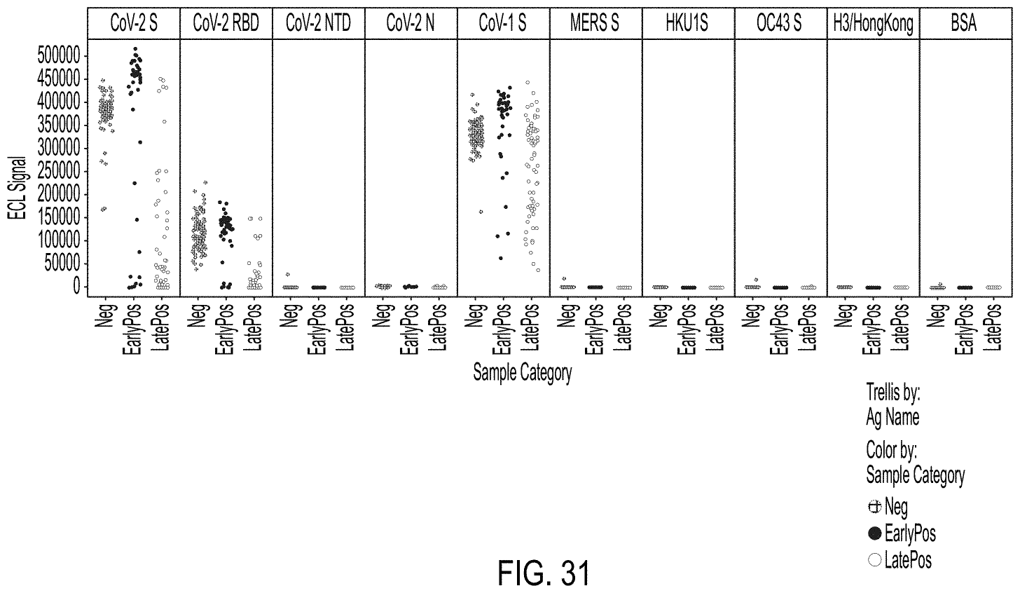

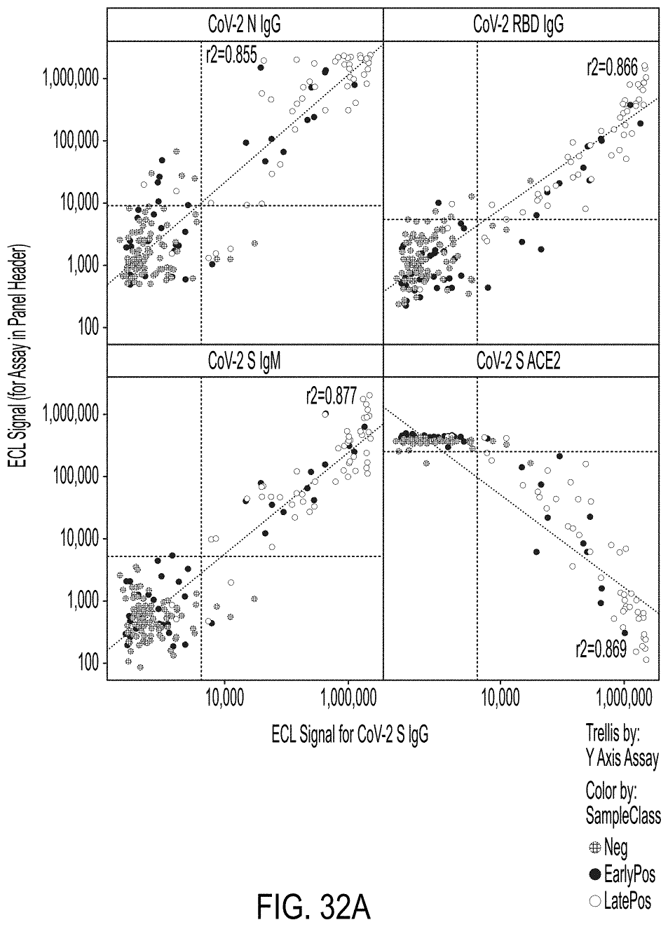

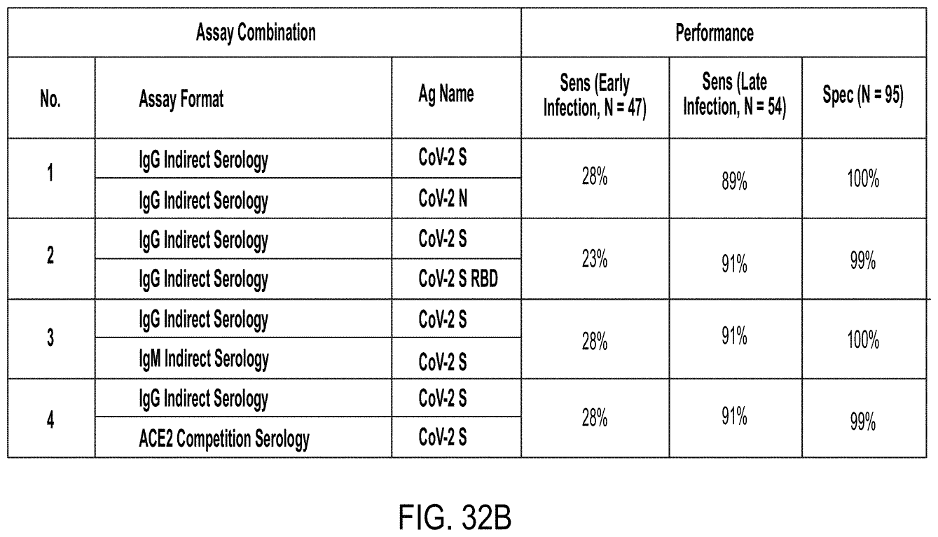

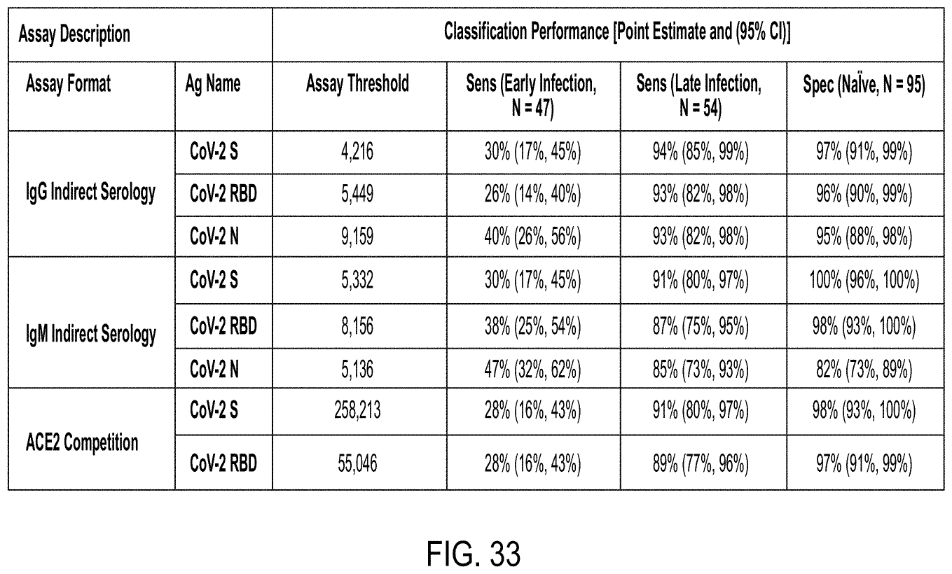

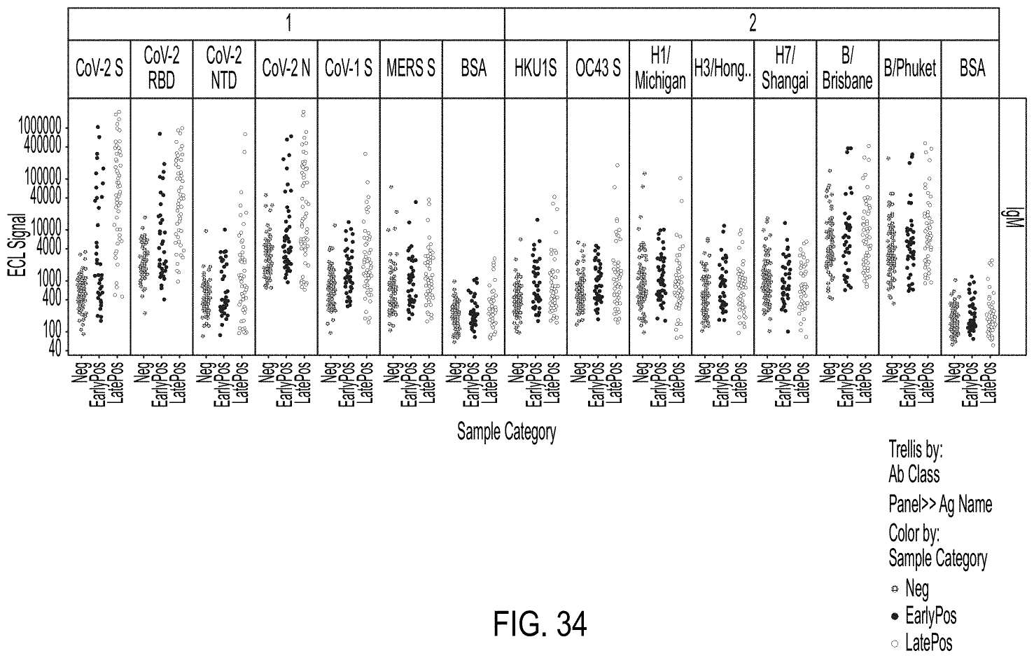

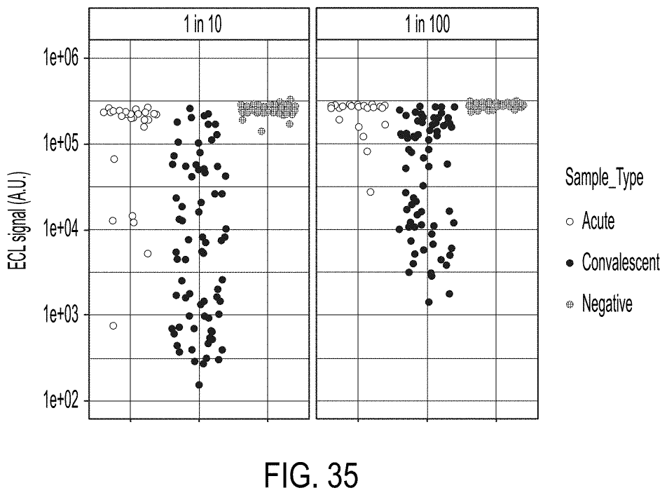

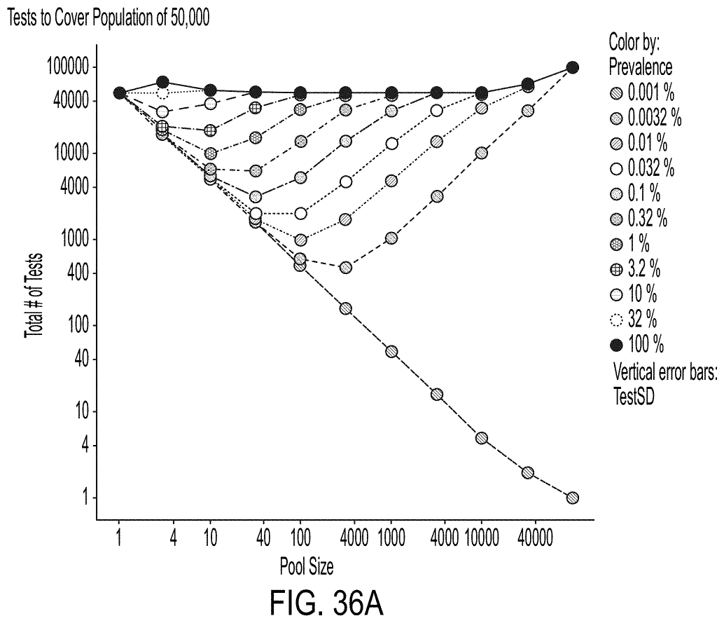

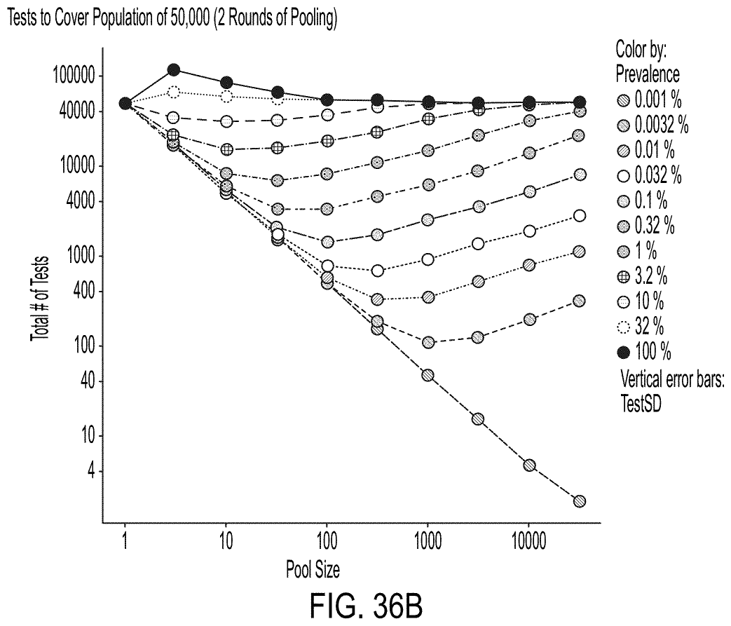

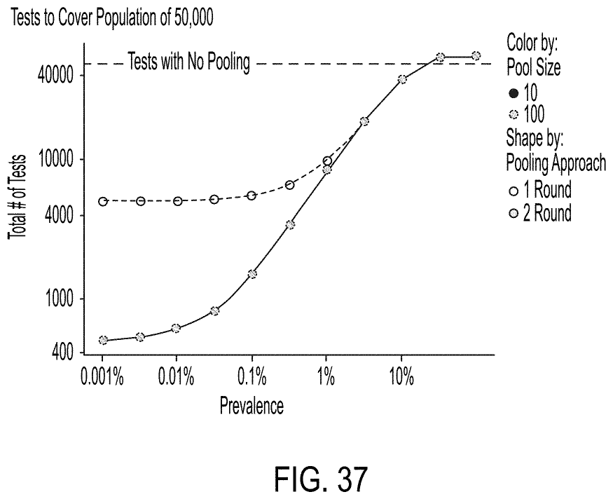

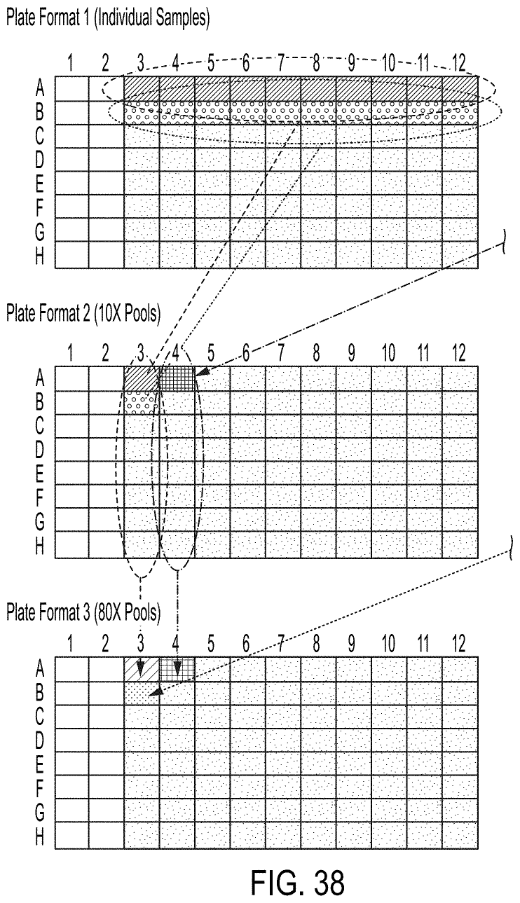

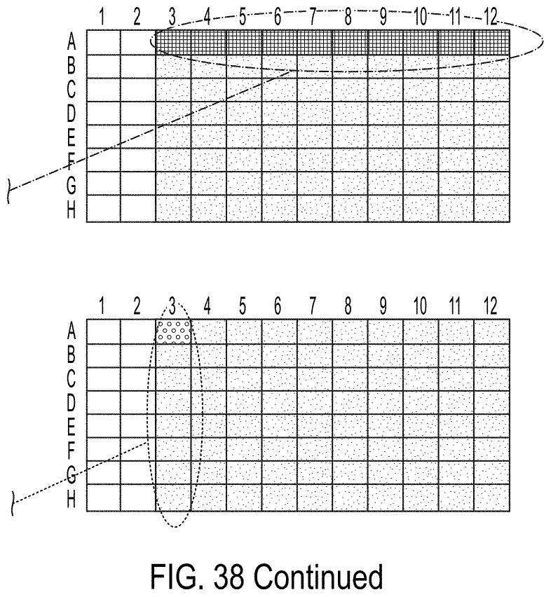

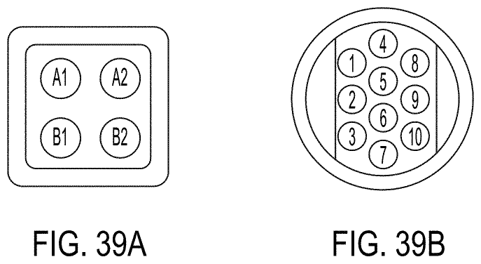

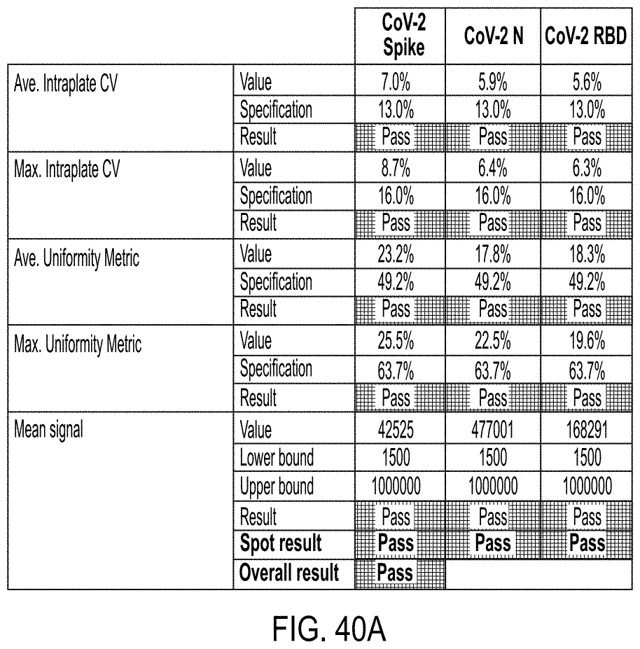

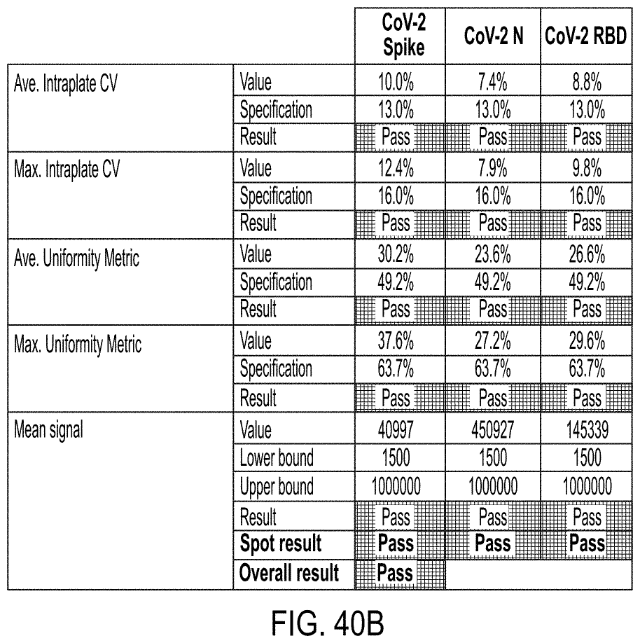

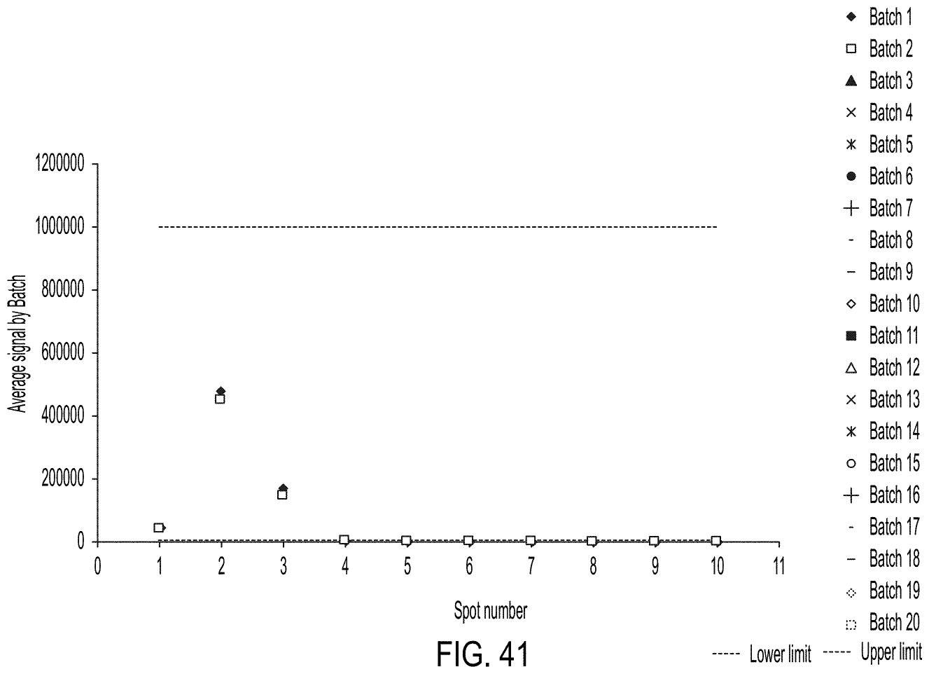

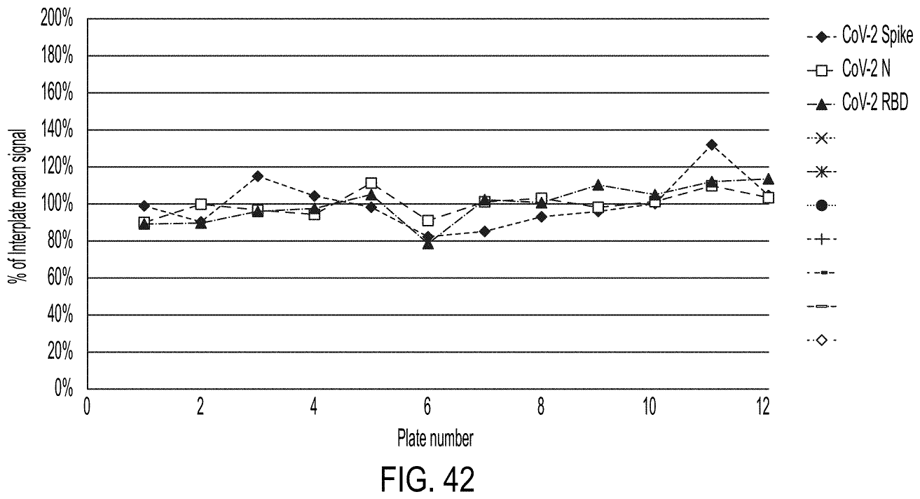

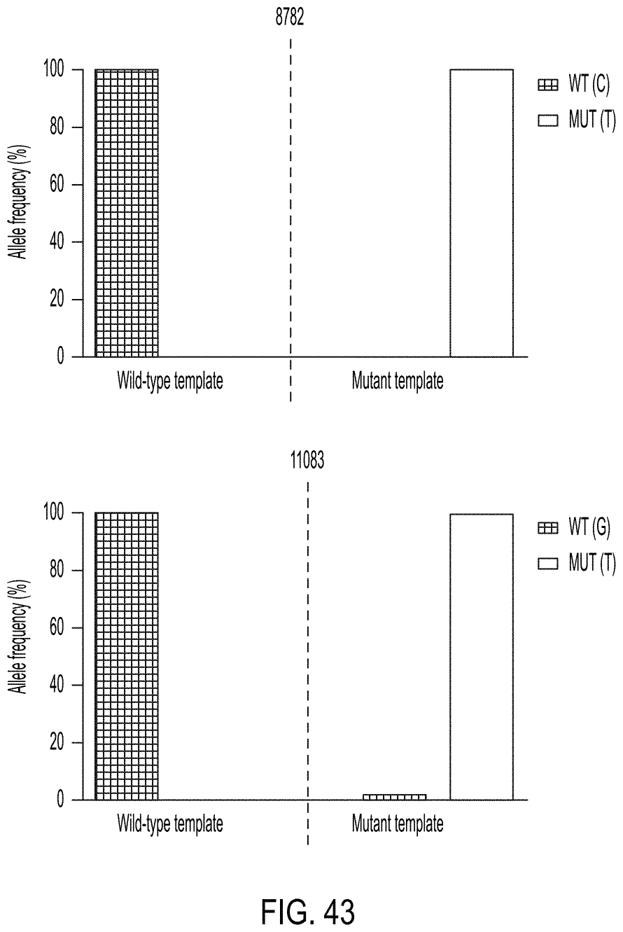

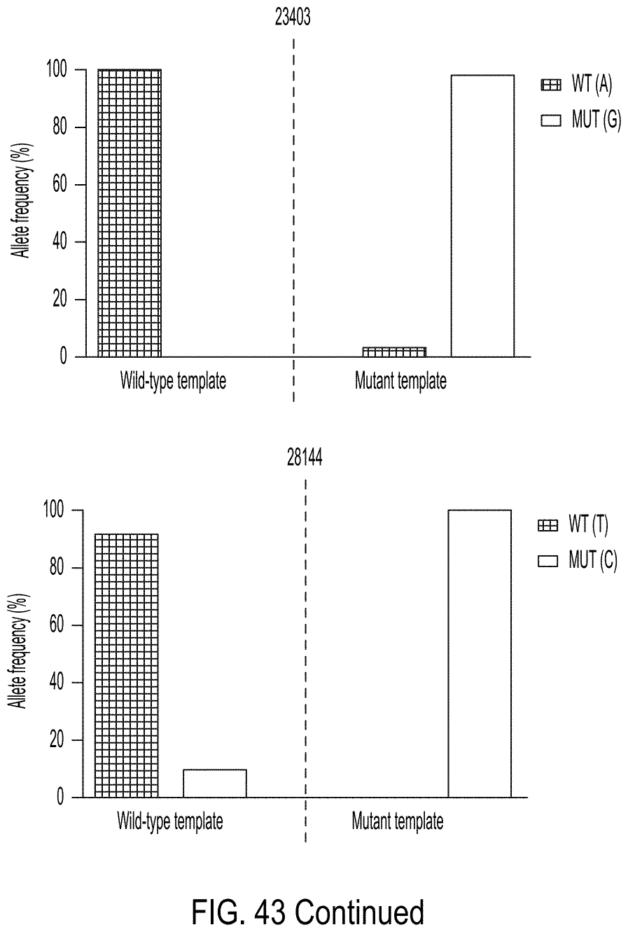

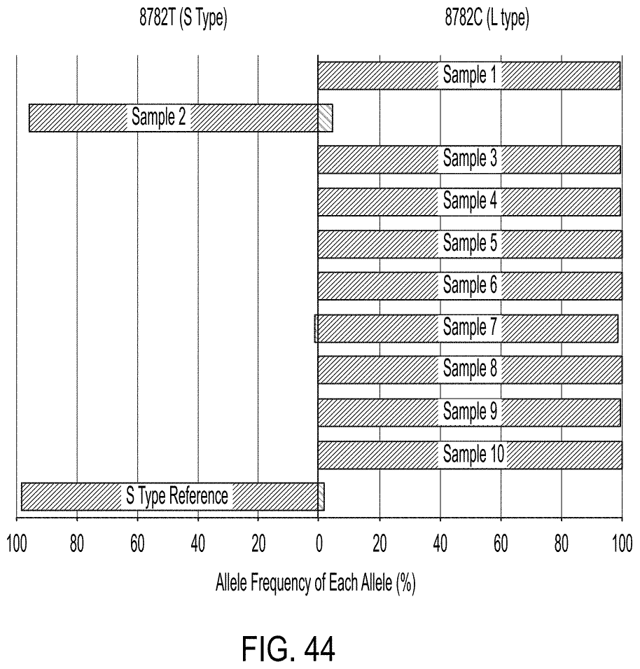

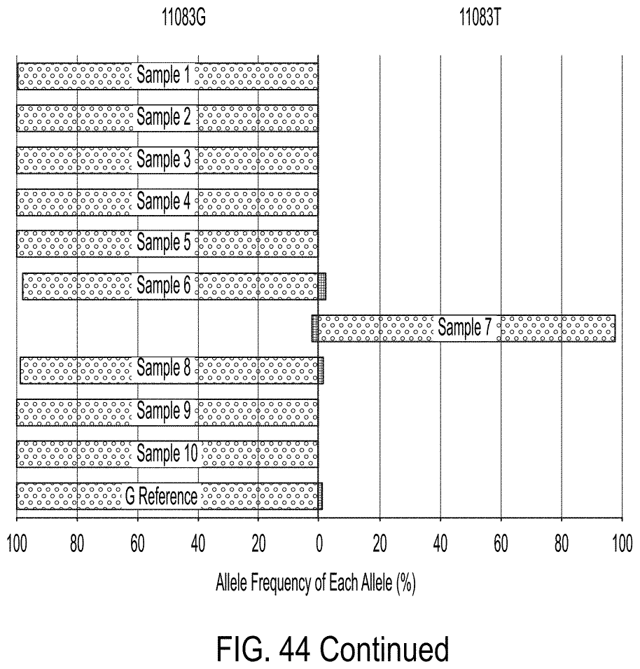

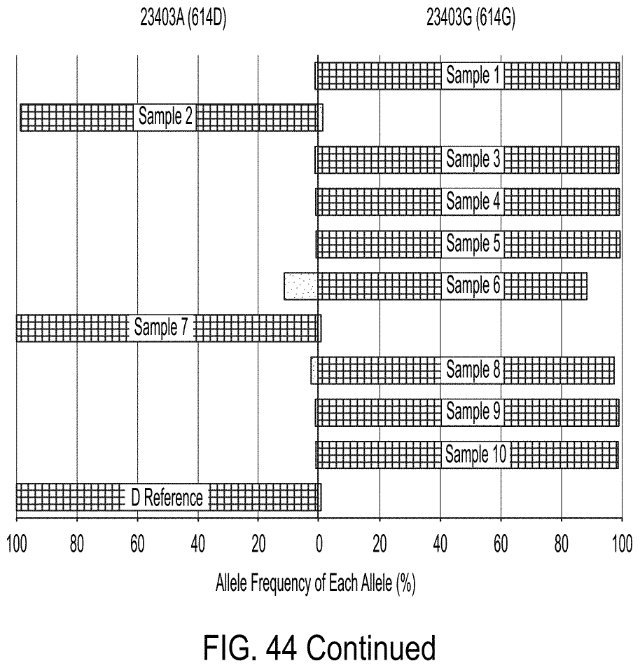

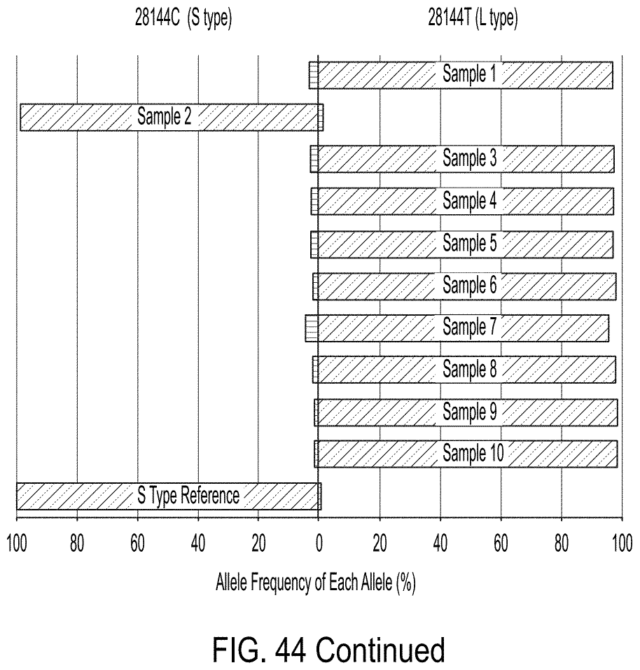

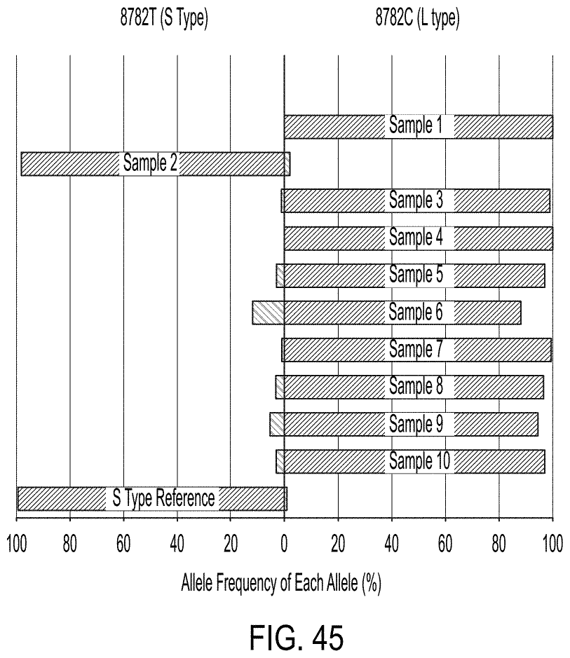

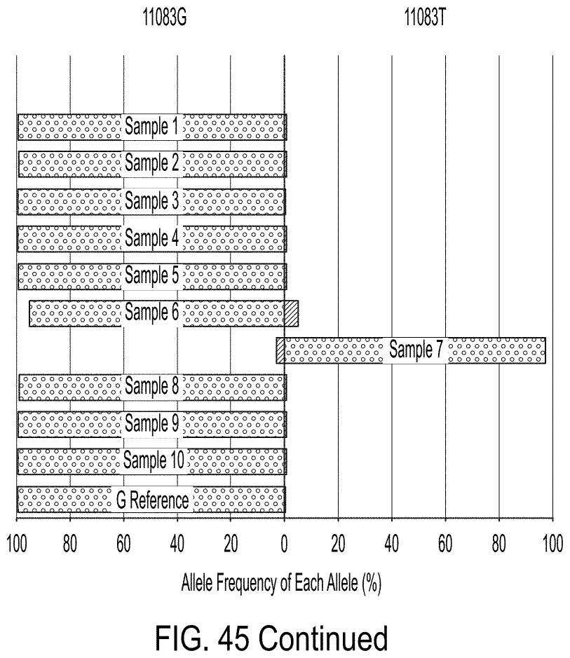

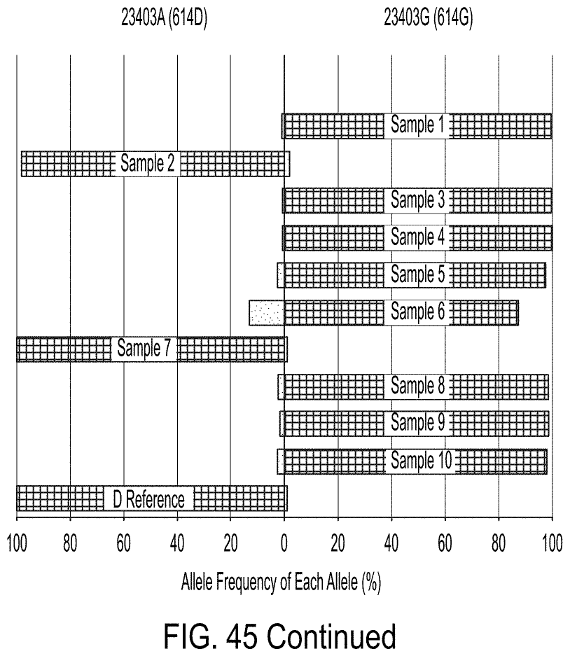

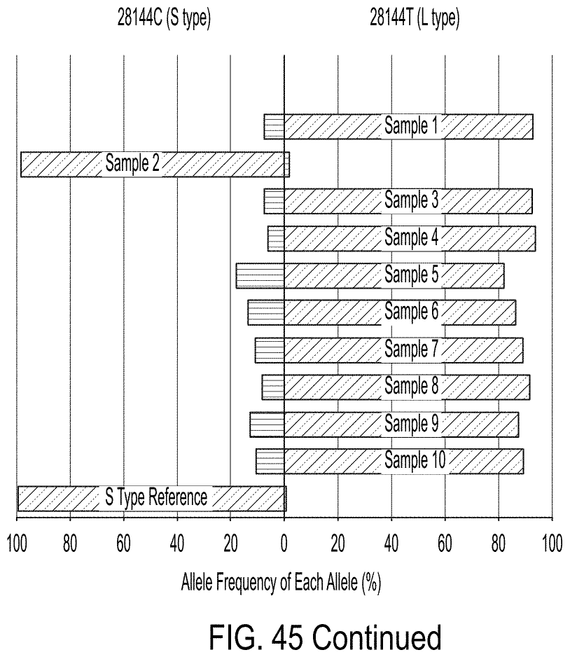

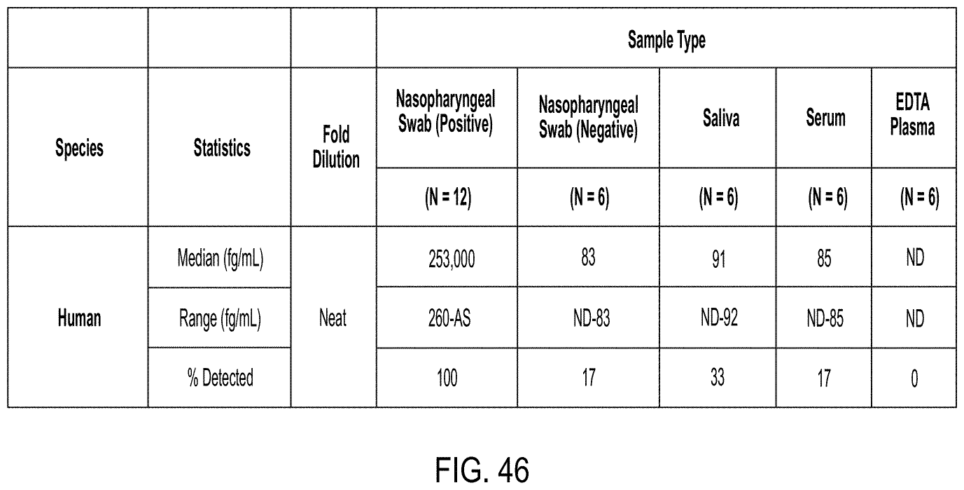

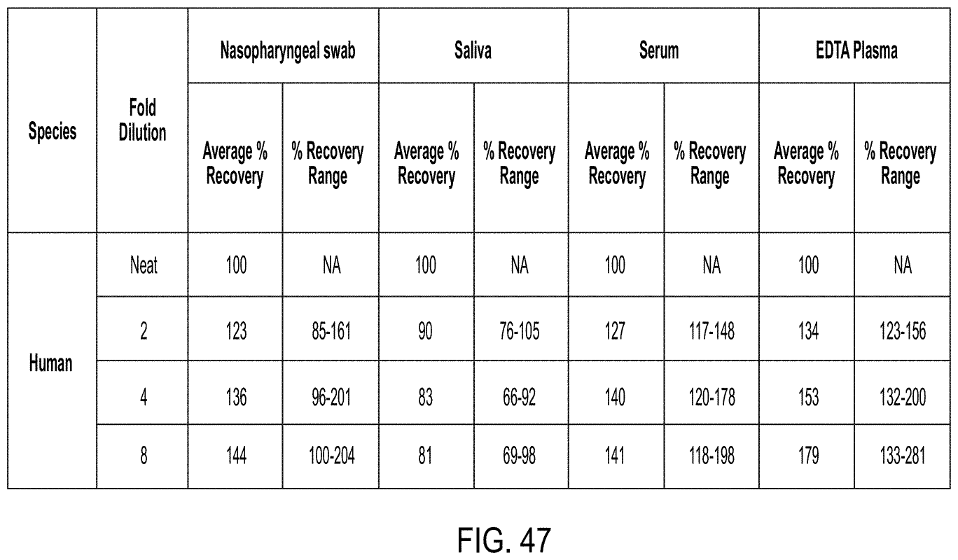

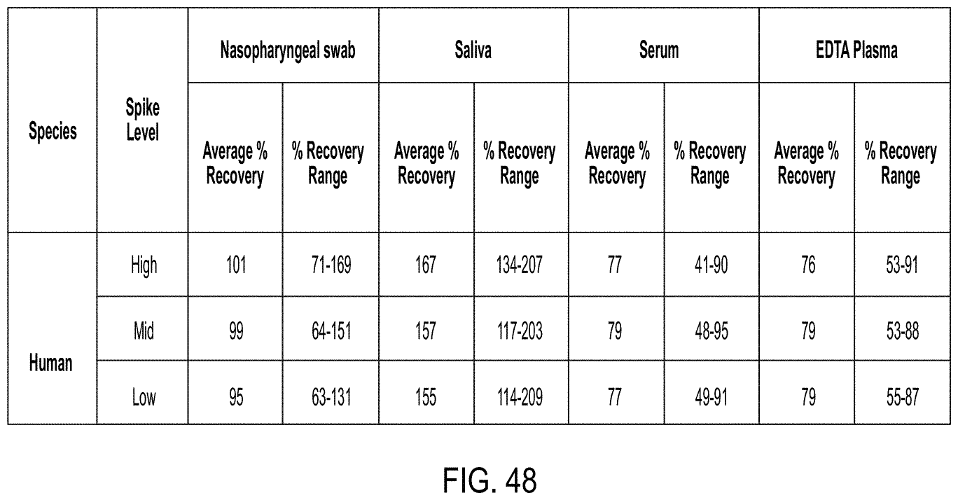

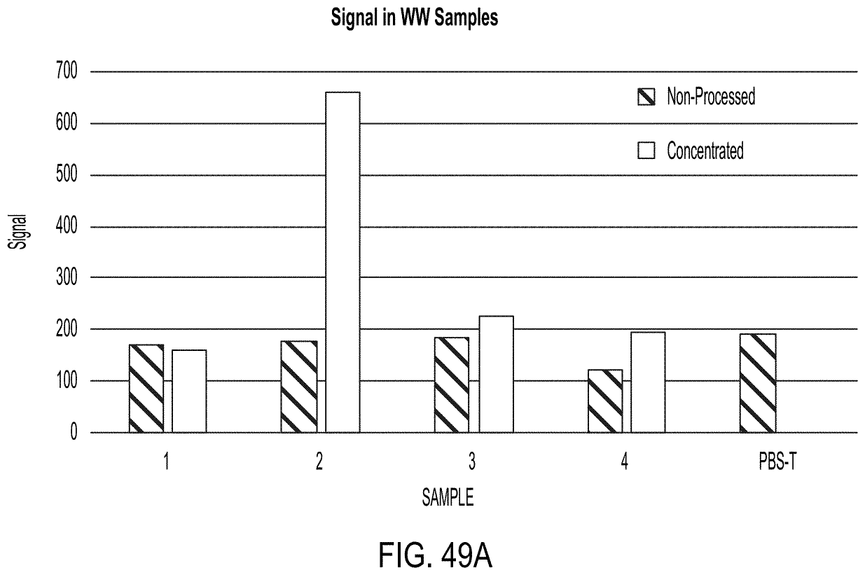

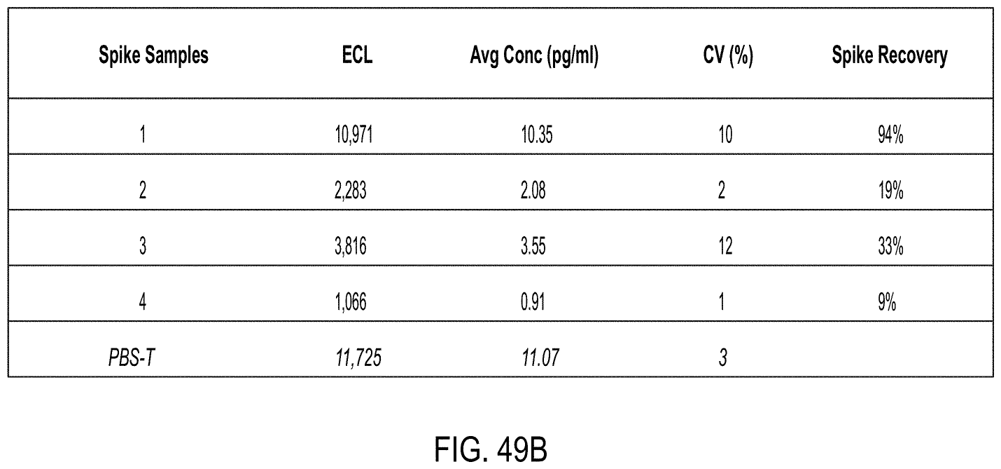

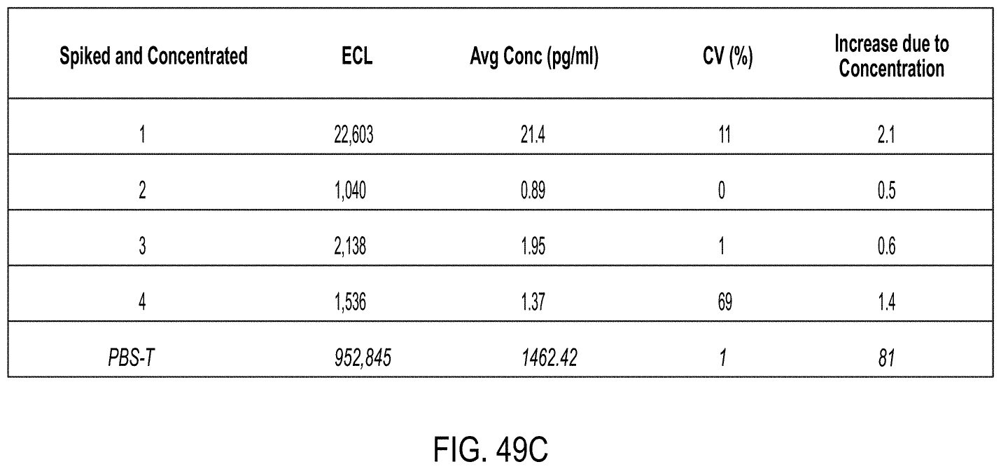

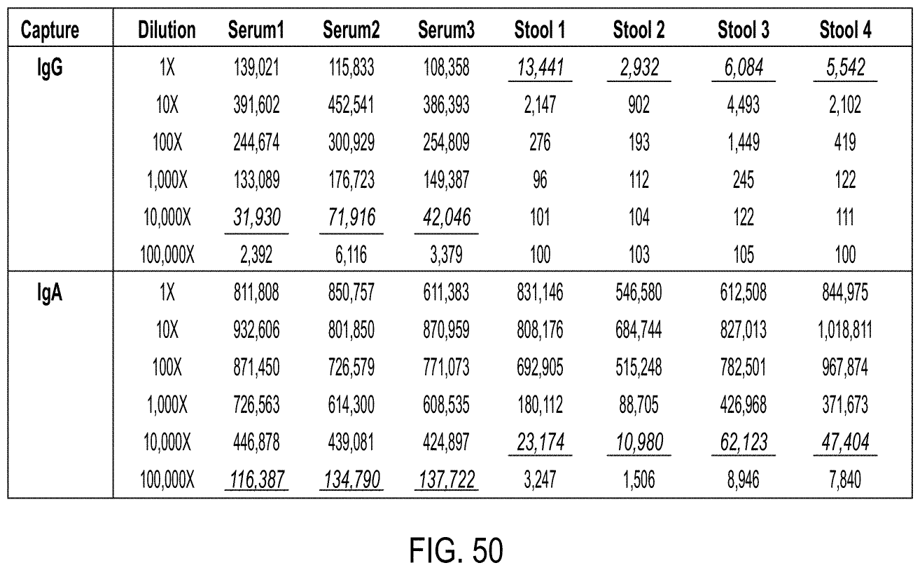

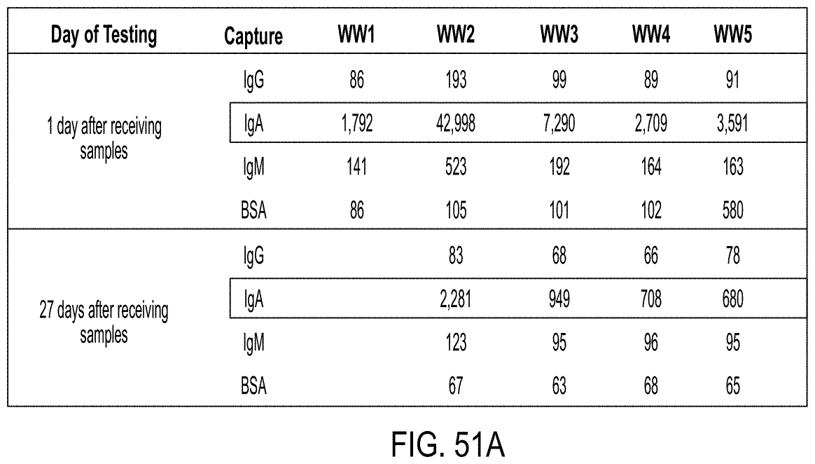

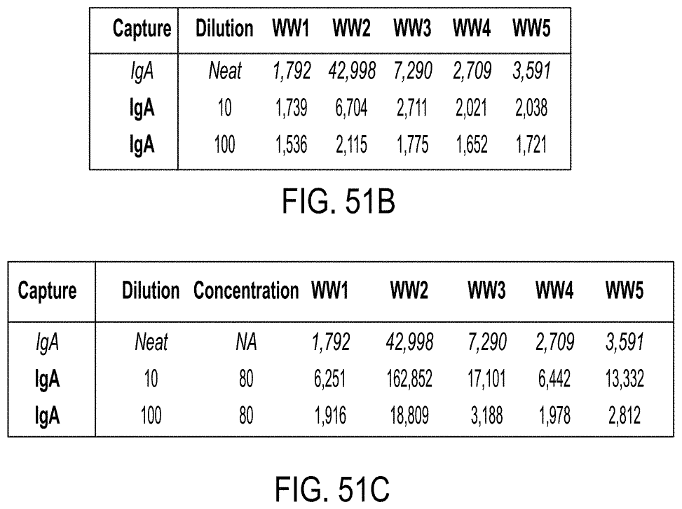

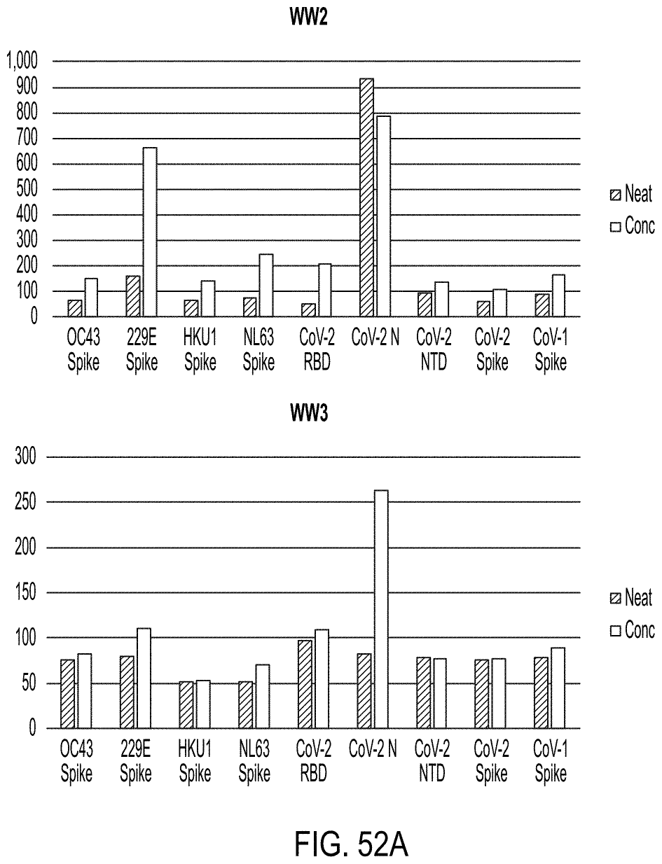

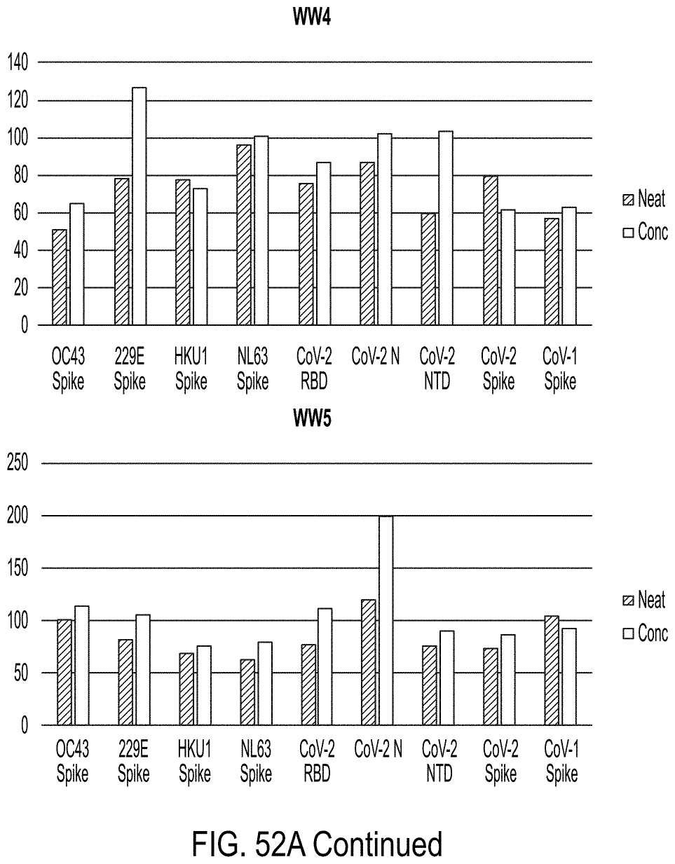

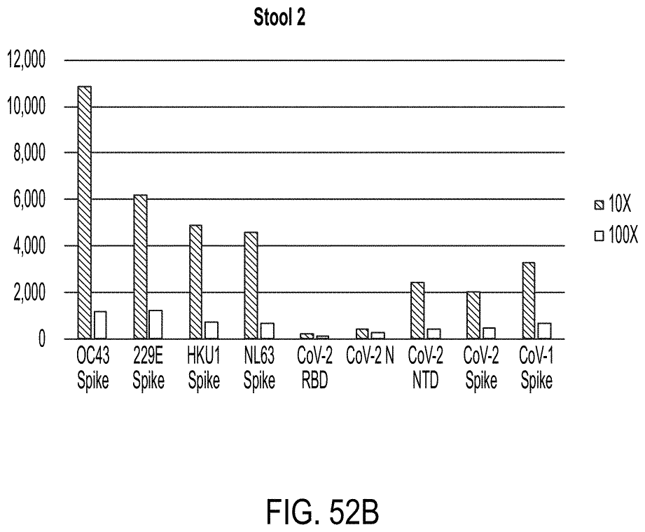

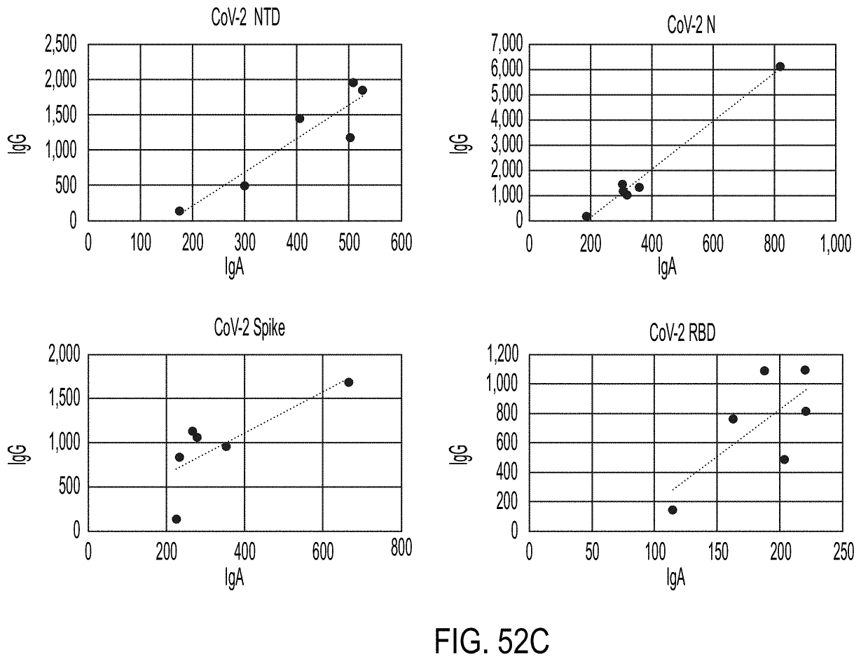

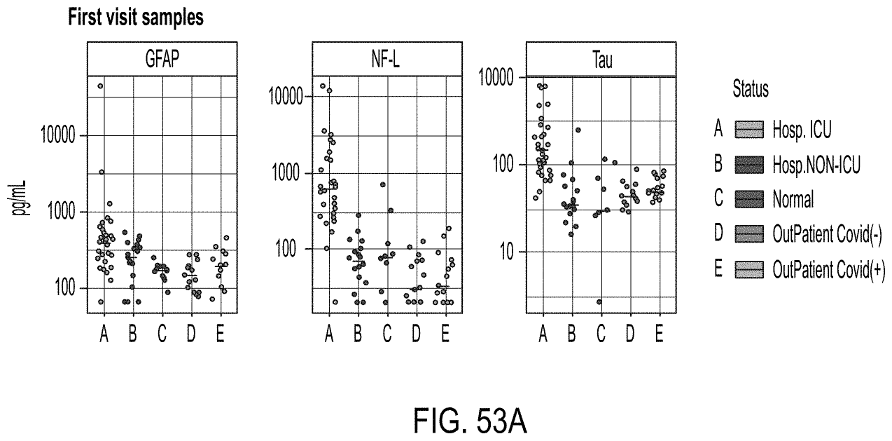

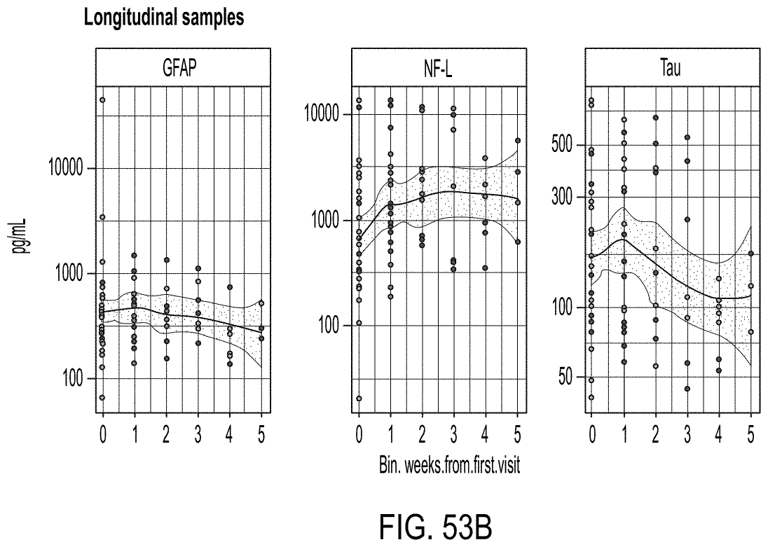

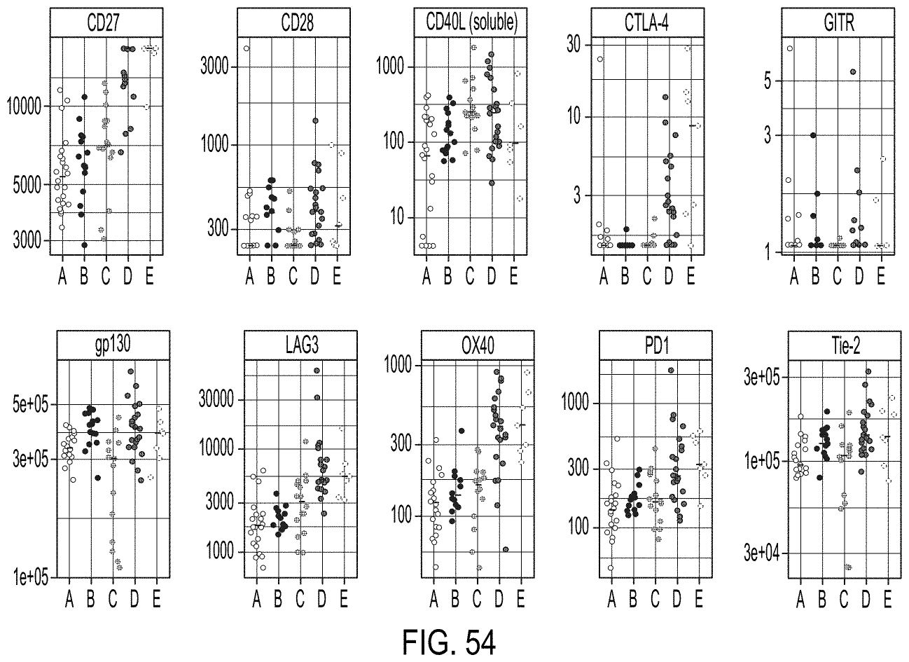

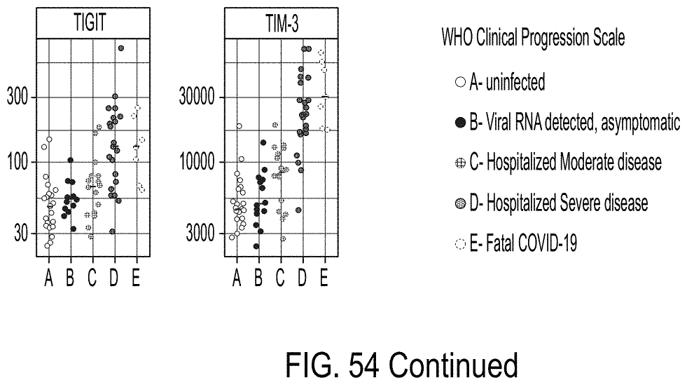

In an alternative embodiment of the method illustrated in FIG. 7, the binding complex comprising the binding reagent and analyte nucleic acid, e.g., coronavirus nucleic acid, is contacted with a Cas nickase. The Cas nickase nicks the binding reagent to remove the secondary TAC and amplification blocker to form a cleaved binding reagent, thereby activating the cleaved binding reagent for amplification and causes the cleaved binding reagent to dissociate from the analyte nucleic acid. In an “amplification cycle” of amplifying the number of cleaved binding reagents, the analyte nucleic acid binds to an additional copy of the binding reagent, which is cleaved by the Cas nickase to form an additional copy of the cleaved binding reagent activated for amplification. After one or more amplification cycles to form a plurality of cleaved binding reagents, the reaction mixture sample is incubated on a secondary surface comprising a secondary targeting agent, which removes any cleaved amplification blocker-secondary targeting agent complement and uncleaved binding reagent. Following the incubation on the secondary surface, the reaction mixture sample is then incubated on a surface comprising a targeting agent to immobilize the plurality of cleaved binding reagents onto the surface. The immobilized cleaved binding reagent(s) is then subjected to extension and detection as described herein.

FIG. 8 illustrates an embodiment of the methods described herein for detecting a viral nucleic acid. An oligonucleotide binding reagent comprises, in 5′ to 3′ order, a targeting agent complement, an amplification primer, a ribonuclease recognition site, and an amplification blocker. Cas13 forms a complex with the target RNA, e.g., coronavirus RNA, and the Cas13 cleaves the target RNA and indiscriminately cleaves the binding reagent to remove the amplification blocker, thereby activating the amplification primer. The reaction mixture sample is incubated on a surface comprising a targeting agent to immobilize the binding complex onto the surface. The immobilized binding complex is then subjected to extension and detection as described herein.

FIGS. 9A-9D illustrate an embodiment of the methods described herein for detecting a single nucleotide polymorphism (SNP) in a viral nucleic acid. In FIGS. 9A-9C, a target nucleic acid (1) that comprises an SNP (2) is contacted with: a targeting probe (3) that comprises an oligonucleotide tag (4) and a sequence that is complementary to the SNP, and a detection probe (5) that comprises detectable label (6). The targeting and detection probes (3, 5) hybridize to the target nucleic acid, and the targeting and detection probes that hybridize with perfect complementarity at the SNP are ligated to form a ligated target complement (11) comprising the oligonucleotide tag and detectable label. The reaction mixture containing the ligated target complement is contacted with a surface comprising one or more binding reagents (7) immobilized in one or more binding domains (9). A signal (10) is detected if the ligated target complement is immobilized on the surface via hybridization of the complementary oligonucleotides in the oligonucleotide tag and the binding reagent. In FIG. 9D, the targeting probe has a mismatch with the SNP in the target nucleic acid, and thus, hybridization and ligation do not occur.

FIGS. 10 and 11 relate to Example 5B. FIG. 10 shows a titration curve of an embodiment of a bridging serology assay described herein. SARS-CoV-2 S-RBD was immobilized as binding reagent, and labeled S-RBD was used as detection reagent to detect a monoclonal antibody against SARS-CoV-2 S-RBD. In a bridging serology assay, signal increases as the antibody concentration increases.

FIG. 11 shows the results of an embodiment of a bridging serology assay described herein. SARS-CoV-2 S-RBD was immobilized as binding reagent, and labeled S-RBD was used as detection reagent. The bridging serology assay was tested on samples from patients who tested negative for COVID-19 (red), early positive (yellow) for COVID-19, and late positive (green) for COVID-19. Higher signal indicates increased number of antibodies bound to the immobilized and labeled antigens.

FIGS. 12 and 13 relate to Example 6B. FIG. 12 shows a titration curve of an embodiment of a neutralization serology assay described herein. SARS-CoV-2 S. SARS-CoV S, SARS-CoV-2 S-RBD, and HCoV-HKU1 S proteins were immobilized as binding reagents, and labeled ACE2 was used as competitor to detect a neutralizing monoclonal antibody against the S proteins (or RBD fragment) from SARS-CoV and SARS-CoV-2. In a neutralization serology assay, signal decreases as the antibody concentration increases.

FIG. 13 shows the results of an embodiment of a neutralization serology assay described herein. SARS-CoV-2 S. SARS-CoV S, SARS-CoV-2 S-RBD, and HCoV-HKU1 S proteins were immobilized as binding reagents, and labeled ACE2 was used as competitor to SARS-CoV-2 antibodies that may be present. The neutralization serology assay was tested on samples from patients who tested negative for COVID-19 (red), early positive (yellow) for COVID-19, and late positive (green) for COVID-19. Lower signal (generated by competitor) indicates increased number of antibodies bound to the immobilized antigen.

FIG. 14 illustrates an embodiment of the methods described herein for detecting a viral nucleic acid. RNA is extracted from a sample containing an RNA virus (e.g., SARS-CoV-2), and the extracted RNA is converted to cDNA. A “Master Mix” is prepared by combining a forward primer comprising a 5′ binding reagent complement sequence and a cDNA complement sequence, a reverse primer comprising a cDNA reverse complement sequence and a 3′ binding partner of a detectable label, and other PCR components such as dNTPs and DNA polymerase. The cDNA and Master Mix are combined, and PCR is performed for 30 to 40 cycles to form a plurality of PCR products, each PCR product comprising the 5′ binding reagent complement sequence and 3′ binding partner of a detectable label. Each PCR product hybridizes to a binding reagent on a surface. The surface is then contacted with a detectable label, which binds to the PCR product. The PCR product bound to the detectable label is then subjected to detection as described herein.

FIG. 15A illustrates an embodiment of the methods described herein for detecting a viral antigen, indicated by the triangle. A surface comprising a binding reagent for a viral antigen captures the viral antigen. A detection reagent comprising a detectable label also binds to the viral antigen, forming a binding complex on the surface. The binding complex can be detected by methods described herein.

FIG. 15B illustrates an embodiment of the methods described herein for detecting an intact virus. A surface comprising a binding reagent for a first viral antigen captures a virus by binding to the first viral antigen on the viral surface. A pair of detection reagents binds to second and third viral antigens in proximity on the viral surface. The detection reagents can include nucleic acid probes, which can be extended to form an extended oligonucleotide, and the extended oligonucleotide is bound to the surface and detected as described herein.

FIG. 15C illustrates an embodiment of the methods described herein for detecting an EV. A surface comprising a binding reagent for a viral antigen captures an intact EV comprising a viral antigen on the EV surface. A pair of detection reagents binds to two host proteins in proximity on the EV surface. The detection reagents can include nucleic acid probes, which can be extended to form an extended oligonucleotide, and the extended oligonucleotide is bound to the surface and detected as described herein.

FIGS. 16 and 17 relate to Example 11. FIGS. 16A and 16B show the results of an embodiment of a serology assay described herein. In FIG. 16A, SARS-CoV-2 N protein, SARS-CoV-2 S protein, SARS-CoV-2 S-RBD, SARS-CoV-2 S-NTD, SARS-CoV S protein, and MERS-CoV S protein were immobilized as binding reagents. In FIG. 16B, HCoV-HKU1 S protein. HCoV-OC43 S protein, FluA H1 (Michigan strain) HA protein, FluA H3 (Hong Kong strain) HA protein, FluA H7 (Shanghai strain) HA protein, FluB (Brisbane strain) HA protein, and FluB (Phuket strain) HA protein were immobilized as binding reagents. Labeled anti-IgG antibody was used to detect IgG in negative, early positive, and late positive SARS-CoV-2 patient samples.

FIG. 17 shows the results of an embodiment of a serology assay described herein. SARS-CoV-2 N protein and SARS-CoV-2 S protein were immobilized as binding reagents. Labeled anti-IgG antibody was used to detect IgG in negative, early positive, and late positive SARS-CoV-2 patient samples.

FIG. 18 relates to Example 12. FIG. 18 shows the correlation between embodiments of serology assays described herein.

FIGS. 19A-19D relate to Example 13. FIG. 19A shows the results of an embodiment of an oligonucleotide ligation assay (OLA) for detection of single nucleotide polymorphism described herein, performed on a synthetic template DNA sequence. FIGS. 19B to 19D show the results of an embodiment of an oligonucleotide ligation assay for detection of single nucleotide polymorphism described herein, performed on samples obtained from patients positive for COVID-19 and a SARS-CoV-2 S-variant control sample. FIG. 19B shows the results of the OLA at SARS-CoV-2 location 8782. FIG. 19C shows the results of the OLA at SARS-CoV-2 location 28144. FIG. 19D shows a summary of the results in FIGS. 19B and 19C and the allelic frequency of the SNPs.

FIGS. 20A-20I relate to Example 14. FIG. 20A shows the measured ECL signal from an exemplary serology assay with a Serology Panel of antigens described herein. FIGS. 20B and 20C show the measured concentration and measured ECL signal, respectively, from an exemplary immunoassay with a Proinflammatory Panel of antigens described herein. FIGS. 20D and 20E show the measured concentration and measured ECL signal, respectively, from an exemplary immunoassay with a Chemokine Panel of antigens described herein. FIGS. 20F and 20G show the measured concentration and measured ECL signal, respectively, from an exemplary immunoassay with a Vascular Injury Panel of antigens described herein. FIGS. 20H and 20I show the measured concentration and measured ECL signal, respectively, from an exemplary immunoassay with an Angiogenesis Panel of antigens described herein. The assay samples were obtained from patients known to be positive (Sample Sets 1A, 2A, and 2B) or negative (Sample Sets 1B and 3) for COVID-19.

FIGS. 21A-21D relate to Example 14. FIGS. 21A-21D show the correlation of results from the Proinflammatory Panel (FIG. 21A), Chemokine Panel (FIG. 21B), Vascular Injury Panel (FIG. 21C), and Angiogenesis Panel (FIG. 21D) with the Serology Panel.

FIGS. 22A and 22B relate to Example 15. FIG. 22A shows the results of an exemplary immunoassay for detecting EVs in samples purified from the plasma of patients known to be negative (samples 1-6) or positive (samples 7-12) for COVID-19, using a TSPAN Panel of binding reagents described herein. FIG. 22B shows the results of an exemplary immunoassay for detecting EVs in samples using a Spike/TSPAN Panel of binding reagents described herein.

FIG. 23 relates to Example 15. FIG. 23 shows the results (average ECL signal and average measured concentration) of an exemplary immunoassay for detecting SARS-CoV-2 N and S proteins in EV samples purified from the plasma of patients known to be negative (samples 1-6) or positive (samples 7-12) for COVID-19, as described herein.

FIGS. 24A and 24B relate to Example 16. FIG. 24A shows the results (average ECL signal, intra-plate CV (coefficient of variation), and inter-plate CV) of an exemplary uniformity test for immobilization of three panels of His6-tagged (SEQ ID NO: 547) viral antigens on a 96-well plate, using an anti-His6 antibody. FIG. 24B shows the results of an exemplary antigen immobilization verification test to determine whether the antigens were immobilized in the correct binding domains on the 96-well plate.

FIG. 25 relates to Example 16. FIG. 25 shows the results (average intra-plate CV, maximum intra-plate CV, and mean ECL signal) of an exemplary uniformity test for immobilization of three panels of viral antigens on a 96-well plate, using a blend of serum samples from subjects known to be COVID-19 positive.

FIG. 26 relates to Example 17. FIG. 26 shows the results (average intra-plate CV, maximum intra-plate CV, mean ECL signal, and CV of intra-plate averages) of an exemplary uniformity test for immobilization of His6-tagged (SEQ ID NO: 547) viral antigens on three lots of 96-well plates, using an anti-His6 antibody.

FIGS. 27A and 27B relate to Example 17. FIGS. 27A and 27B show the results (FIG. 27A: specific ECL signal; FIG. 27B: % specific binding) of an exemplary antigen immobilization verification test to determine whether the antigens were immobilized in the correct binding domains on the 96-well plates.

FIGS. 28A-28C relate to Example 17. FIGS. 28A-28C show the correlation between plate lots of the signals for each of the immobilized viral antigens (FIG. 28A: N protein; FIG. 28B: S protein; FIG. 28C: S-RBD), as measured in Example 17.

FIG. 29 relates to Example 4B. FIG. 29 shows the results of an exemplary serology assay with a Pandemic CoV Panel described herein, using samples containing monoclonal antibodies against SARS-CoV-2 N and S proteins.

FIG. 30 relates to Example 6C. FIG. 30 shows the results of an exemplary neutralization serology assay with a Mixed Panel of viral antigens described herein, using samples containing a monoclonal antibody against SARS-CoV.

FIG. 31 relates to Example 6D. FIG. 31 shows the results of an exemplary neutralization serology assay with a Mixed Panel of viral antigens described herein, using patient samples that had been tested to be negative, early positive, and late positive for COVID-19.

FIGS. 32A and 32B relate to Example 12. FIG. 32A shows the correlation results of the indirect serology assays for IgG against SARS-CoV-2 S with four other serology assays: IgG against SARS-CoV-2 N, IgG against SARS-CoV-2 S-RBD, IgM against SARS-CoV-2 S, and ACE2 competitor assay. FIG. 32B shows the assay performance (sensitivity and specificity) for the assay pairings shown in FIG. 32A.

FIG. 33 relates to Example 18. FIG. 33 shows the assay performance (sensitivity at early and late infections and specificity) of IgG indirect serology assay and IgM indirect serology assay (described in Example 4B) and ACE2 competitor assay (described in Example 6D).

FIG. 34 relates to Example 11. FIG. 34 shows the results of an exemplary serology assay for detecting IgM using the viral antigens in Panels 1 and 2: SARS-CoV-2 N protein, SARS-CoV-2 S protein, SARS-CoV-2 S-RBD, SARS-CoV-2 S-NTD, SARS-CoV S protein, and MERS-CoV S protein; HCoV-HKU1 S protein, HCoV-OC43 S protein, FluA H1 (Michigan strain) HA protein, FluA H3 (Hong Kong strain) HA protein, FluA H7 (Shanghai strain) HA protein, FluB (Brisbane strain) HA protein, and FluB (Phuket strain) HA protein.

FIG. 35 relates to Example 6E. FIG. 35 shows the results of an exemplary neutralization serology assay for detecting antibodies against SARS-CoV-2 in serum samples that were COVID-19 negative; obtained from acute COVID-19 patients; or obtained from convalescent COVID-19 patients. Results are shown for 1:10 sample dilution (left panel) and 1:100 sample dilution (right panel).

FIGS. 36A and 36B illustrate examples of the total number of tests needed for a population of 50,000 individuals using different pooled sample sizes, wherein the disease prevalence in the population varies from 0.001% to 100% when using a single-layer pooling strategy (FIG. 36A) or a two-layer pooling strategy (FIG. 36B) as described in embodiments herein.

FIG. 37 illustrates an example of three testing approaches for a population of 50,000 individuals based on disease prevalence, as described in embodiments herein. The first approach uses a two-layer pooling strategy with a pool size of 100 individual samples with (filled circles): the second approach uses a single-layer pooling strategy with a pool size of 10 individual samples (open circles); the third approach uses no pooling (dashed line).

FIG. 38 illustrates an exemplary approach for performing a two-layered pooling strategy in a 96-well plate, as described in embodiments herein. Eighty (80) individual samples are contained in the shaded wells (rows A-H and columns 3-12) Plate Format 1 (PF1). The individual samples in PF1 are combined according to Plate Format 2 (PF2) to create pooled samples containing 10 individual samples. The pooled samples of PF2 are further combined according to Plate Format 3 (PF3) to create pooled samples containing 80 individual samples.

FIGS. 39A and 39B illustrate exemplary assay surfaces described in embodiments herein. FIG. 39A shows a well of an exemplary 384-well assay plate, comprising four distinct binding domains (“spots”). FIG. 39B shows a well of an exemplary 96-well assay plate, comprising ten distinct binding domains (“spots”).

FIGS. 40A-42 relate to Example 22. FIGS. 40A and 40B show the results (average intra-plate CV, maximum intra-plate CV, mean ECL signal, and CV of intra-plate averages) of an exemplary anti-His6 antibody-based uniformity test for immobilization His6-tagged (SEQ ID NO: 547) viral antigens on two batches containing a total of 12 plates.

FIG. 41 shows the mean ECL signal results from the immobilized antigens on two batches of plates, as tested with an exemplary anti-His6 antibody-based uniformity test.

FIG. 42 shows the ECL signals from each of the three antigens (CoV-2 Spike, CoV-2 N and CoV-2 RBD) on the plates as a percentage of the inter-plate mean signal, as tested with an exemplary anti-His6 antibody-based uniformity test.

FIGS. 43-45 relate to Example 23. FIG. 43 shows the results from an exemplary oligonucleotide ligation assay (OLA) for detection of SARS-CoV-2 single nucleotide polymorphisms (SNPs) at genome locations 8782, 11083, 23403, and 28144, with a synthetic template oligonucleotide.

FIG. 44 shows the results of an exemplary singleplex OLA assay for detecting SARS-CoV-2 SNPs at genome locations 8782, 11083, 23403, and 28144, with samples obtained from SARS-CoV-2 positive patients.

FIG. 45 shows the results of an exemplary multiplex OLA assay for detecting SARS-CoV-2 SNPs at genome locations 8782, 11083, 23403, and 28144, with samples obtained from SARS-CoV-2 positive patients.

FIGS. 46-48 relate to Example 24. FIG. 46 shows the results of an exemplary assay for measuring the concentration (fg/mL) of SARS-CoV-2 nucleocapsid (N) protein from the following samples: nasopharyngeal swabs from 12 patients who tested positive for COVID-19, nasopharyngeal swabs from 6 patients who tested negative for COVID-19, and normal (COVID-19 negative) human saliva, serum, and EDTA plasma.

FIG. 47 shows the percent recovery results of an exemplary test to assess dilution linearity of the SARS-CoV-2 N protein detection assay. The normal human serum, EDTA plasma, saliva, and COVID-19 negative human nasopharyngeal swab samples were spiked with calibrator and tested at different dilutions.

FIG. 48 shows the percent recovery results of an exemplary test to assess spike recovery of the SARS-CoV-2 N protein detection assay. The normal human serum. EDTA plasma, saliva, and COVID-19 negative human nasopharyngeal swab samples were spiked with calibrator at three levels.

FIGS. 49A-52C relate to Example 27. FIG. 49A shows the results of an exemplary immunoassay for the detection of SARS-CoV-2 N protein in five wastewater samples. FIG. 49B shows the spike recovery results of the wastewater samples spiked with heat-inactivated SARS-CoV-2. FIG. 49C shows the spike recovery results of the wastewater samples spiked with heat-inactivated SARS-CoV-2 and concentrated with a 10K spin filter.

FIG. 50 shows the results of an exemplary immunoassay to determine the amounts of IgG and IgA in stool as compared to serum. Underlined numbers indicate a linear drop in signal upon dilution.

FIGS. 51A-51C show the results of an exemplary immunoassay performed on wastewater samples after 1 day or 27 days of storage to determine the amount of total IgA, IgG, and IgM. FIG. 51A shows the measured signals for IgA, IgG, and IgM from five wastewater samples. FIG. 51B shows the measured IgA signals at three different dilution levels. FIG. 51C shows the measured IgA signals from samples that have been concentrated. Concentrated samples were diluted prior to performing the assay.

FIGS. 52A-52C show the results of an exemplary serology assay (Panel #15 as shown in Table 6 of Example 3) performed on four wastewater samples and one stool sample collected from an individual prior to the emergence of SARS-CoV-2, to determine the amounts of antibodies against SARS-CoV-2 antigens. FIG. 52A shows the measured IgA signals in the four wastewater samples. FIG. 52B shows the measured antibody signals in the stool sample collected from an individual prior to the emergence of SARS-CoV-2. FIG. 52C shows the relationship between signals for IgA and IgG antibodies against CoV-2 related antigens measured in six concentrated waste water samples.

FIGS. 53A-53B show the results of an exemplary biomarker assay to assess levels of GFAP, total Tau, and NF-L, performed on plasma samples from hospitalized COVID-19 patients and outpatients testing positive or negative for COVID-19. FIG. 53A shows the measured biomarker levels at the time of the patient's initial hospital visit. FIG. 53B shows the measured biomarker levels at 0 to 5 weeks after the initial hospital visit.

FIG. 54 shows the results of an exemplary biomarker assay to assess levels of CD27, CD28, CD40L, CTLA-4, GITR, gp130, LAG3, OX40, PD1, Tie-2, TIGIT, and TIM-3, performed on samples from subjects who were uninfected with COVID-19, subjects who had asymptomatic COVID-19 infection as determined by a positive PCR test, and subjects who were hospitalized from a COVID-19 infection and classified as having a moderate infection, severe infection, or fatal infection.

### DETAILED DESCRIPTION OF THE INVENTION

The disclosed embodiments fulfill the urgent need for high-quality viral assays and methods useful for the COVID-19 pandemic. Disclosed embodiments have been widely adopted for COVID-19 research, epidemiology, and vaccine development and have had a significant impact on the COVID-19 public health response. For example, serology embodiments are widely used (e.g., Johnson M et al. J Clin Virol 2020; 130:104572; Corbett K S et al. N Engl J Med 2020:383:1544-55; Folegatti P M et al. The Lancet 2020; 396:467-78; Ramasamy M N et al. The Lancet 2020; 396:1979-93; Goldblatt D et al. J Hosp Infect 2021; 110:60-6; Majdoubi A et al. JCI Insight 2021, doi.org/10.1172/jci.insight.146316; Amjadi M F et al. MedRxiv 2021:2021.01.05.21249240, doi.org/10.1101/2021.01.05.21249240; Grandjean L et al. MedRxiv 2020:2020.07.16.20155663, doi.org/10.1101/2020.07.16.20155663; Majdoubi A et al. MedRxiv 2020:2020.10.05.20206664, doi.org/10.1101/2020.10.05.20206664). Certain embodiments disclosed herein were chosen by the United States government initiative. Operation Warp Speed, as the basis of its standard binding assay for immunogenicity assessments in all funded Phase III clinical trials of vaccines. Serology assay embodiments (e.g., assays to detect immunoglobulin(s) conducted on non-bodily samples or bodily samples (e.g., serum, plasma, saliva)) disclosed herein aid in assessing human immune responses to COVID-19 infection and vaccination and in understanding the interplay between COVID-19 and immunity to other coronaviruses and respiratory pathogens. The disclosed nucleic acid detection embodiments have advantages over PCR methods. e.g., in their speed, simplicity, cost, and high throughput. The disclosed intact virus detection embodiments provide improved accuracy and specificity of an active infection diagnosis as compared to detection of an individual viral component. Serology assays, nucleic acid detection assays, and other embodiments related to mutations and variants of SARS-CoV-2 are proving important as new mutations and variants arise. Other biomarker detection embodiments disclosed herein, e.g., detection of inflammatory and/or tissue damage response biomarkers and/or extracellular vesicles. e.g., from virus-infected cells, have wide applicability, regardless of viral mutation status, to studies on morbidity and mortality to understand factors underlying severe illness, death, and persistent symptoms following acute infection and may lead to better interventions. Data showing the high-quality nature of the disclosed embodiments are described in the Examples and elsewhere herein.

Immunoassays described herein for the detection of respiratory viruses, including coronaviruses, provide numerous advantages compared with nucleic acid amplification (e.g., PCR) based detection methods. For example, immunoassays are conducted in a simple and streamlined format with improved sensitivity. Improved sensitivity with immunoassays occurs because these assays not only detect viral particles, but also individual viral proteins in damaged tissue being cleared by the body at the site of infections. Moreover, immunoassays for biomarkers produced by the body in response to infection (e.g., antibodies against the virus or inflammatory factors associated with the host response to infection) take advantage of the natural amplification associated with the immune response.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### Respiratory Virus Detection

In embodiments, the invention provides an immunoassay method for detecting at least one respiratory virus, including a coronavirus, in a biological sample. As used herein, a “respiratory virus” refers to a virus that can cause a respiratory tract infection, e.g., in a human. Exemplary respiratory viruses include, but are not limited to, coronavirus, influenza virus, respiratory syncytial virus (RSV), paramyxovirus, adenovirus, parainfluenza virus (PIV), bocavirus, metapneumovirus, orthopneumovirus, enterovirus, rhinovirus, and the like. Respiratory virus infections can be difficult to diagnose because different viruses can often cause similar symptoms in a patient. For example, coughing and low-grade fever are typical symptoms of early disease progression or mild cases of a coronavirus infection (e.g., COVID-19), as well as influenza or a respiratory syncytial virus (RSV) infection. An assay that can simultaneously test for several potential causes of infection would advantageously allow a respiratory virus infection to be correctly and efficiently diagnosed in a single assay run and utilizing a single patient sample. In embodiments, the methods herein distinguish between and among different types of a given virus (e.g., distinguishing PIV-1, PIV-2, PIV-3, and PIV-4 from each other or influenza A from influenza B from each other), as well as between and among different subtypes or strains (e.g., distinguishing influenza A (H1N1) from influenza A (H3N2)).

In embodiments, the invention provides an immunoassay method for detecting at least one respiratory virus in a biological sample, comprising: (a) contacting the biological sample with a binding reagent that specifically binds a component of at least one respiratory virus in the biological sample; (b) forming a binding complex comprising the binding reagent and the respiratory virus component; and (c) detecting the binding complex, thereby detecting the at least one respiratory virus in the biological sample.

In embodiments, the at least one respiratory virus comprises a coronavirus, an influenza virus, a paramyxovirus, an adenovirus, a bocavirus, a pneumovirus, an enterovirus, a rhinovirus, or a combination thereof. Exemplary coronaviruses and methods for their detection are described herein and include, but are not limited to, SARS-CoV (also known as SARS-CoV-1), MERS-CoV, SARS-CoV-2, HCoV-OC43, HCoV-229E, HCoV-NL63, HCoV-HKU1. In embodiments, the method detects a coronavirus by detecting a coronavirus nonstructural protein, e.g., nsp1, nsp2, nsp3, nsp4, nsp5, nsp6, nsp7, nsp8, nsp9, nsp10, nsp11, nsp12, nsp13, nsp14, nsp15, or nsp16. In embodiments, the method detects a coronavirus by detecting a coronavirus structural protein, e.g., the E, S (including S1, S2, S-NTD, S-ECD, and S-RBD), M, HE, or N proteins. Coronaviruses and their proteins are further described herein.

Exemplary influenza viruses include, but are not limited to, influenza A (FluA), influenza B (FluB), and influenza C (FluC). Typically, the seasonal flu is caused by FluA and/or FluB. FluA viruses can be further characterized into various subtypes based on the hemagglutinin (HA) and neuraminidase (N) proteins present on the surface of the viral particle, e.g., H1N1, H1N2, H2N2, H3N2, H5N1, H7N2, H7N3, H7N7, H9N2, and H10N7. FluA strains include, e.g., H1/Michigan strain, H3/Hong Kong strain, H7/Shanghai strain, and the like. FluB viruses can be further characterized into genetic lineages, e.g., the FluB (Victoria) or FluB (Yamagata) viruses. In embodiments, the immunoassay detects an influenza virus component, e.g., an influenza virus-specific protein. In embodiments, the immunoassay detects an influenza structural protein. In embodiments, the immunoassay detects an influenza nonstructural protein. In embodiments, the immunoassay detects an influenza virus by detecting the influenza HA protein. In embodiments, the immunoassay detects an influenza virus by detecting the influenza N protein. In embodiments, the immunoassay detects an influenza virus by detecting an influenza nucleoprotein (NP). In embodiments, the immunoassay detects a FluA virus and is further capable of determining the subtype of the FluA virus. In embodiments, the immunoassay detects a FluB virus and is further capable of determining the lineage of the FluB virus.

Exemplary paramyxoviruses include, but are not limited to, parainfluenza virus type 1, parainfluenza virus type 2, parainfluenza virus type 3, and parainfluenza virus type 4. In embodiments, the immunoassay detects a paramyxovirus component, e.g., a paramyxovirus-specific protein. In embodiments, the immunoassay detects a paramyxovirus structural protein. In embodiments, the immunoassay detects a paramyxovirus nonstructural protein. Non-limiting examples of paramyxovirus proteins that can be detected by the immunoassay include a nucleocapsid (N) protein, transcriptase (L), phosphoprotein (P), fusion protein (F), hemagglutinin-neuraminidase (HN) or hemagglutinin (H), and non-glycosylated membrane protein (M).

Adenoviruses that can cause respiratory infections include, but are not limited to, adenovirus type 3, type 4, and type 7. In embodiments, the immunoassay detects an adenovirus component, e.g., an adenovirus-specific protein. In embodiments, the immunoassay detects an adenovirus structural protein. In embodiments, the immunoassay detects an adenovirus nonstructural protein. Non-limiting examples of adenovirus proteins that can be detected by the immunoassay include a capsid protein, encapsidation protein, L3 protease, E1A, E1B, E2A, E2B, E3, and E4.

Exemplary bocaviruses include, but are not limited to, HBoV1, HBoV2, HBoV3, and HBoV4. In embodiments, the immunoassay detects a bocavirus component, e.g., a bocavirus-specific protein. In embodiments, the immunoassay detects a bocavirus structural protein. In embodiments, the immunoassay detects a bocavirus nonstructural protein. Non-limiting examples of bocavirus proteins that can be detected by the immunoassay include NS1, NS2. NS3, NS4, VP1, VP2, and VP3.

Exemplary pneumoviruses include, but are not limited to, respiratory syncytial virus (RSV), including human respiratory syncytial virus B1 (HRSV-B1) and human respiratory syncytial virus A2 (HRSV-A2). In embodiments, the immunoassay detects a pneumovirus component, e.g., a pneumovirus-specific protein. In embodiments, the immunoassay detects a pneumovirus structural protein. In embodiments, the immunoassay detects a pneumovirus nonstructural protein. Non-limiting examples of pneumovirus proteins that can be detected by the immunoassay include fusion (F), attachment (G), lipoprotein (SH), nucleoprotein (N), phosphoprotein (P), membrane protein (M), and large protein (L).

Exemplary enterovirus include, but are not limited to, EV-A, EV-B, EV-C, including EV-C104, EV-C105, EV-C109, EV-C117, EV-C118, and EV-D, including EV-D68. In embodiments, the immunoassay detects an enterovirus component, e.g., an enterovirus-specific protein. In embodiments, the immunoassay detects an enterovirus structural protein. In embodiments, the immunoassay detects an enterovirus nonstructural protein. Non-limiting examples of enterovirus proteins that can be detected by the immunoassay include the capsid proteins VP1, VP2, VP3, and VP4, nonstructural proteins 2A, 2B, 2C, 3A, 3B, 3C, and 3D, and VPg.

Exemplary rhinoviruses include, but are not limited to, RV-A, RV-B, and RV-C. In embodiments, the immunoassay detects a rhinovirus component, e.g., a rhinovirus-specific protein. In embodiments, the immunoassay detects a rhinovirus structural protein. In embodiments, the immunoassay detects a rhinovirus nonstructural protein. Non-limiting examples of rhinovirus proteins that can be detected by the immunoassay include the capsid proteins VP1, VP2, VP3, and VP4, nonstructural proteins 2A, 2B, 2C, 3A, 3B, 3C, and 3D, and VPg.

In embodiments, the method detects SARS-CoV, MERS-CoV, SARS-CoV-2, HCoV-OC43, HCoV-229E, HCoV-NL63, HCoV-HKU1, influenza A, influenza B, RSV, or a combination thereof. In embodiments, the method is a multiplexed method capable of simultaneously detecting one or more of SARS-CoV. MERS-CoV. SARS-CoV-2, HCoV-OC43, HCoV-229E, HCoV-NL63, HCoV-HKU1, influenza A, influenza B, and RSV. In embodiments, the method further comprises repeating one or more of the method steps described herein to detect one or more respiratory viruses in the sample. In embodiments, the method further comprises repeating steps (a)-(c) of the method described herein, wherein each detected respiratory virus comprises a component that binds to a different binding reagent, thereby detecting the at least one respiratory virus. In embodiments, each of steps (a)-(c) is performed for each respiratory virus in parallel.

As used herein, the term “simultaneous” in reference to one or more events (e.g., detection of one or more viruses, viral components, or biomarkers as described herein) means that the events occur at exactly the same time or at substantially the same time, e.g., simultaneous events described herein can occur less than or about 30 minutes apart, less than or about 20 minutes apart, less than or about 15 minutes apart, less than or about 10 minutes apart, less than or about 5 minutes apart, less than or about 2 minutes apart, less than or about 1 minute apart, or less than or about 30 seconds apart. In the context of embodiments of multiplexed immunoassays provided herein, “simultaneous” refers to detecting a on single surface (e.g., a particle, an assay plate, an assay cartridge, or a well of a multi-well assay plate) the presence of one or more viruses, viral components or biomarkers described herein. In embodiments, a multiplexed assay is performed on a single assay plate. In embodiments, a multiplexed assay is performed in a single well of an assay plate. In embodiments, a multiplexed assay is performed in a single assay cartridge. In embodiments, a multiplexed immunoassay is performed on more than one assay plates. In embodiments, more than one multiplexed immunoassay (e.g., wherein each multiplexed immunoassay detects a combination of biomarkers and/or viral components as described herein) is performed on a single surface, e.g., a single well of an assay plate or a single assay cartridge. The number of assay wells and/or assay plates that may be required to perform a multiplexed assay can be determined, e.g., based on the number of substances of interest to be detected in one or more samples (e.g., a multiplex of 2, 3, 4, 5, 6, 7, 8, 9, 10, or more viruses, viral components, and/or biomarkers described herein); the number of samples being assayed (e.g., from one or more subjects); the number of calibration reagents being measured to generate a calibration curve (e.g., 3, 4, 5, 6, 7, 8, 9, 10, 11, 12, or more); the number of control reagents being measured (e.g., 0, 1, 2, 3, or more); the number of replicates for each sample, calibration reagent, and/or control reagent being measured (e.g., singlicate, duplicate, triplicate, or more); and the number of wells per assay plate (e.g., 6, 12, 48, 96, 384, or 1536 wells per assay plate). When multiplexed immunoassay is conducted on multiple assay plates, the assay plates can be read simultaneously or at different times. The timing of reading the assay plates can be determined, e.g., based on the capacity of the assay reader instrument (e.g., capable of reading 1, 2, 3, 4, or more plates at once); the read-time of the assay reader instrument (e.g., about 1 s to about 600 s, about 10 s to about 500 s, about 20 s to about 300 s, about 30 s to about 180 s, about 60 s to about 120 s, about 70 s, or about 90 s per assay plate); the time required to prepare the assay components (e.g., about 10 s, 20 s, 30 s, 1 min, 2 min, 5 min, 10 min, 15 min, 30 min, 1 hr, or more per plate); and the equipment for performing the assay (e.g., a single-channel pipettor may require a longer time for pipetting the assay components as compared to a multichannel pipettor; handling liquids from different containers, e.g., tubes, vials, or plates, may require different lengths of time). In embodiments, “simultaneous” refers to events occurring with respect to a single sample (e.g., a biological sample in a single vial or container from a single subject) or replicates or dilutions of a single sample. Factors affecting the timing of simultaneous events include the following: the number of multiplexed assays being performed at the same time on a single sample (e.g., a multiplex of 2, 3, 4, 5, 6, 7, 8, 9, 10, or more assays in a single well or cartridge); the number of assay modules in a panel (e.g., 1, 2, 3, or more plates or cartridges in a panel); the number of samples being assayed at the same time (e.g., a number of samples capable of being assayed in one kit or more than one kit); the number of points on a calibration curve (e.g., 5, 6, 7, 8, 9, 10, 12, or more); the presence and number of controls (e.g., 0, 1, 2, 3, or more controls); the read-time of the instrument (e.g., about 1 s to about 600 s, about 10 s to about 500 s, about 20 s to about 300 s, about 30 s to about 180 s, about 60 s to about 120 s, about 70 s, or about 90 s); the number of replicates of each calibrator, control, or sample (e.g., singlicate, duplicate, triplicate, or more); the number of wells per plate (e.g., 6, 12, 48, 96, 384, or 1536 wells per plate); and/or the type of equipment for performing the assay (e.g., a single channel or a multi channel pipettor, tubes or plates for dilution).

In embodiments, the binding reagent that specifically binds to the respiratory virus component described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof. In embodiments, the binding reagent is a receptor for the respiratory virus component. In embodiments, the binding reagent is a binding partner of the respiratory virus component. In embodiments, the binding reagent is angiotensin-converting enzyme 2 (ACE2). In embodiments, the binding reagent is a neuropilin (NRP) receptor. In embodiments, the binding reagent is NRP1. In embodiments, the binding reagent is NRP2.

### Coronavirus Detection

Coronaviruses, which belong to the Coronaviridae family of viruses, are enveloped viruses with a positive-sense single-stranded RNA genome and a nucleocapsid of helical geometry. A characteristic feature of coronaviruses is the club-shaped spikes that project from the virus surface. In general, a coronavirus particle is assembled from its structural proteins, including an envelope (E), a spike glycoprotein (S), which includes S1 and S2 subunits that form the ectodomain (S-ECD), a viral membrane protein (M), a hemagglutinin-esterase dimer (HE), nucleocapsid (N), and RNA. The S protein comprises a N-terminal domain (N-Term or NTD). The S1 subunit comprises a receptor binding domain (S-RBD), which binds a host receptor (e.g., ACE2) during infection. The S1 subunit can also bind to the cell surface neuropilin-1 (NRP1) receptor. See, e.g., Daly et al., bioRxiv 2020.06.05. 134114 (2020) doi:10.1101/2020.06.05.134114. In embodiments, coronavirus S proteins, including recombinantly expressed S proteins and variants thereof, are further described, e.g., in WO 2018/081318. For example, two variants of SARS-CoV-2 each has a single polynucleotide morphism (SNP) at genome location 23403, which is in the gene encoding the S protein, resulting in a different amino acid at position 614 of the S protein: D614 and G614 (denoted as S: 23403A>G, D614G; see. e.g., Korber et al., bioRxiv 2020.04.29. 069054 (2020) doi:10.1101/2020.04.29.069054; also published as Korber et al., Cell 182(4):P812-827 (2020)), referred to herein respectively as S-D614 and S-D614G. Further mutations of the SARS-CoV-2 S protein are described in Tables 1A and 1B. Sequence alignments between the genetic material of various coronavirus species have also revealed additional conserved open reading frames for Coronaviruses also encode a number of nonstructural proteins (NSPs), which are expressed in infected cells but are generally not incorporated into the viral particle itself. Exemplary coronavirus NSPs include, but are not limited to, nsp1, nsp2, nsp3, nsp4, nsp5, nsp6, nsp7, nsp8, nsp9 (replicase), nsp10, nsp11, nsp12 (multi-domain RNA polymerase), nsp13 (helicase. RNA 5′ triphosphatase), nsp14 (N7-methyl transferase, exonuclease), nsp15 (endoribonuclease), nsp16 (2′-O-methyl transferase), and the like. See, e.g., Snijder et al., Adv Virus Res 96:59-126 (2016); Fehr et al., Coronaviruses 1281:1-23 (2015). Sequence alignments between the genetic material of various coronavirus species have revealed conserved open reading frames for several structural and nonstructural proteins, e.g., N, M, S, nsp1, nsp3, nsp6, nsp7, and nsp8. See, e.g., Grifoni et al., bioRxiv 2020.02.12.946087 (2020) doi:10.1101/2020.02.12.

While assays for a specific coronavirus species can identify infection by that particular coronavirus, such assays may have limited usefulness when new strains of infectious coronaviruses emerge. In embodiments, the invention provides a method for detecting a coronavirus in a sample by detecting a conserved coronavirus component, e.g., a protein that is generally conserved across all coronavirus species. Such a method would enable detection of novel coronaviruses of interest.

In embodiments, the invention provides an immunoassay method for detecting a coronavirus in a biological sample, comprising: a) contacting the biological sample with a binding reagent that specifically binds a component of the coronavirus; b) forming a binding complex comprising the binding reagent and the coronavirus component; and c) detecting the binding complex, thereby detecting the coronavirus in the biological sample. In embodiments, the method detects SARS-CoV, MERS-CoV, SARS-CoV-2, HCoV-OC43, HCoV-229E, HCoV-NL63, HCoV-HKU1, or a combination thereof. In embodiments, the biological sample is saliva.

In embodiments, the coronavirus component is on the outer surface of the viral particle. In embodiments, the coronavirus component is integrated in the membrane of the viral particle. In embodiments, the coronavirus component is a protein. In embodiments, the coronavirus component comprises a sugar, e.g., a glycoprotein. In embodiments, the coronavirus component is a structural protein. In embodiments, the coronavirus component is an envelope (E) protein. In embodiments, the coronavirus component is a spike glycoprotein (S) or a variant or subunit thereof, e.g., S-D614, S-D614G, or any of the S protein variants in Tables 1A and 1B, subunit 1 (S1), subunit 2 (S2), ectodomain (S-ECD), N-terminal domain (S-NTD or S-N-Term), or receptor binding domain (S-RBD). In embodiments, the S protein subunit (e.g., SL, S2. S-ECD. S-NTD, or S-RBD) comprises a mutation as described in Tables 1A and 1B. In embodiments, the coronavirus component is a viral membrane (M) protein. In embodiments, the coronavirus component is a hemagglutinin-esterase dimer (HE). In embodiments, the coronavirus component is a nucleocapsid (N) protein. In embodiments, the coronavirus component comprises a mutation as described in Table 1A.

In embodiments, the coronavirus component is a non-structural protein. In embodiments, the coronavirus component is nsp1, nsp2, nsp3, nsp4, nsp5, nsp6, nsp7, nsp8, nsp9, nsp10, nsp11, nsp12, nsp13, nsp14, nsp15, or nsp16. In embodiments, the coronavirus component is a protein substantially conserved across coronaviruses. It will be understood by one of ordinary skill in the art that a protein that is “substantially conserved” across a viral family, e.g., the coronavirus family, means that at least 50%, at least 60%, at least 70%, at least 80%, at least 90%, or at least 95% of species in the viral family contains a protein with at least 50%, at least 60%, at least 70%, at least 80%, at least 90%, or at least 95% sequence similarity, structural similarity, or both. Methods and tools for determining sequence and/or structural similarity are known in the field and include. e.g., algorithms such as Align, BLAST, and CLUSTAL for sequence similarity, and TM-align, DALI, STRUCTAL, and MINRMS.

In embodiments, the immunoassay method detects a coronavirus by detecting the coronavirus E protein. In embodiments, the immunoassay method detects a coronavirus by detecting the coronavirus S protein. In embodiments, the immunoassay method detects a coronavirus by detecting the coronavirus S1 protein subunit. In embodiments, the immunoassay method detects a coronavirus by detecting the coronavirus S2 protein subunit. In embodiments, the immunoassay method detects a coronavirus by detecting the coronavirus S-ECD. In embodiments, the immunoassay method detects a coronavirus by detecting the coronavirus S-RBD. In embodiments, the immunoassay method detects a coronavirus by detecting the coronavirus S-NTD. In embodiments, the immunoassay method detects a coronavirus by detecting the coronavirus M protein. In embodiments, the immunoassay method detects a coronavirus by detecting the coronavirus HE protein. In embodiments, the immunoassay method detects a coronavirus by detecting the coronavirus N protein. In embodiments, the immunoassay method detects a coronavirus by detecting one or more of the coronavirus nsp1, nsp2, nsp3, nsp4, nsp5, nsp6, nsp7, nsp8, nsp9, nsp10, nsp11, nsp12, nsp13, nsp14, nsp15, or nsp16. In embodiments, the immunoassay detects a coronavirus by detecting a combination of the coronavirus proteins described herein. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein. In embodiments, the immunoassay method detects SARS-CoV-2 by detecting SARS-CoV-2 S-D614. In embodiments, the immunoassay method detects SARS-CoV-2 by detecting SARS-CoV-2 S-D614G. In embodiments, the immunoassay method detects SARS-CoV-2 by detecting any of the SARS-CoV-2 S protein variants in Tables 1A and 1B. In embodiments, the immunoassay method detects SARS-CoV-2 by detecting SARS-CoV-2 E protein. In embodiments, the immunoassay method detects SARS-CoV-2 by detecting SARS-CoV-2 M protein. In embodiments, the immunoassay detects SARS-CoV-2 by detecting SARS-CoV-2 N protein and S protein. In embodiments, the immunoassay method detects SARS-CoV-2 by detecting SARS-CoV-2 S protein, N protein, E protein, and M protein. SARS-CoV-2 nonstructural proteins include the Orf1a and Orf1ab replicase/transcriptase proteins; the Orf3a protein; the Orf6a protein; the Orf7a and Orf7b accessory proteins; the Orf8 protein monomer, which is known to form oligomers; and the Orf10 protein. SARS-CoV-2 nonstructural proteins are further described in, e.g., Khailany et al., Gene Rep 19:100682 (2020); and Flower et. al.., Proc Nat Acad Sci 118(2): e2021785118 (2021). In embodiments, the immunoassay detects SARS-CoV-2 by detecting any of SARS-CoV-2 Orf1a, Orf1ab. Orf3a, Orf6a, Orf7a, Orf7b. Orf8 monomer, Orf8 oligomer, Orf10, RNA-dependent RNA polymerase (RdRp), or a combination thereof. In embodiments, the immunoassay method detects SARS-CoV-2 by detecting any of the SARS-CoV-2 protein variants in Table 1A.

In embodiments, the immunoassay method for detecting SARS-CoV-2 comprises: a) contacting the biological sample with a binding reagent that specifically binds a SARS-CoV-2 S protein; b) forming a binding complex comprising the binding reagent and the SARS-CoV-2 S protein; and c) detecting the binding complex, thereby detecting SARS-CoV-2 in the biological sample. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises any of the mutations shown in Tables 1A and 1B. In embodiments, the binding complex further comprises a detection reagent that specifically binds to the SARS-CoV-2 S protein. In embodiments, the detection reagent comprises a detectable label. In embodiments, the detection reagent comprises a nucleic acid probe. Detection reagents are further described herein. In embodiments, the biological sample is saliva.

In embodiments, the immunoassay method for detecting SARS-CoV-2 comprises: a) contacting the biological sample with a binding reagent that specifically binds a SARS-CoV-2 N protein; b) forming a binding complex comprising the binding reagent and the SARS-CoV-2 N protein; and c) detecting the binding complex, thereby detecting SARS-CoV-2 in the biological sample. In embodiments, the SARS-CoV-2 N protein comprises any of the mutations shown in Table 1A. In embodiments, the binding complex further comprises a detection reagent that specifically binds to the SARS-CoV-2 N protein. In embodiments, the detection reagent comprises a detectable label. In embodiments, the detection reagent comprises a nucleic acid probe. Detection reagents are further described herein. In embodiments, the biological sample is saliva.

In embodiments, the immunoassay method for detecting SARS-CoV-2 comprises: a) contacting the biological sample with a binding reagent that specifically binds a SARS-CoV-2 E protein; b) forming a binding complex comprising the binding reagent and the SARS-CoV-2 E protein; and c) detecting the binding complex, thereby detecting SARS-CoV-2 in the biological sample. In embodiments, the SARS-CoV-2 E protein comprises any of the mutations shown in Table 1A. In embodiments, the binding complex further comprises a detection reagent that specifically binds to the SARS-CoV-2 E protein. In embodiments, the detection reagent comprises a detectable label. In embodiments, the detection reagent comprises a nucleic acid probe. Detection reagents are further described herein. In embodiments, the biological sample is saliva.

In embodiments, the immunoassay method for detecting SARS-CoV-2 comprises: a) contacting the biological sample with a binding reagent that specifically binds a SARS-CoV-2 M protein; b) forming a binding complex comprising the binding reagent and the SARS-CoV-2 M protein; and c) detecting the binding complex, thereby detecting SARS-CoV-2 in the biological sample. In embodiments, the binding complex further comprises a detection reagent that specifically binds to the SARS-CoV-2 M protein. In embodiments, the detection reagent comprises a detectable label. In embodiments, the detection reagent comprises a nucleic acid probe. Detection reagents are further described herein. In embodiments, the biological sample is saliva.

In humans, coronaviruses can cause respiratory tract infections ranging from mild to lethal. Infection by the coronaviruses SARS-CoV. MERS-CoV, and SARS-CoV-2 can cause severe respiratory illness symptoms, i.e., severe acute respiratory syndrome (SARS), Middle East respiratory syndrome (MERS), or coronavirus disease 2019 (COVID-19), respectively. Infection by the coronaviruses HCoV-OC43. HCoV-229E, HCoV-NL63, or HCoV-HKU1 can lead to mild respiratory illness symptoms, e.g., the common cold. Coronaviruses can also cause disease in animals such as cats, birds, chickens, cows, and pigs. As used herein, “respiratory tract infection” or “respiratory infection” can refer to an upper respiratory tract infection (URI or URTI) or a lower respiratory tract infection (LRI or LRTI). URTIs include infection of the nose, sinuses, pharynx, and larynx, e.g., tonsillitis, pharyngitis, laryngitis, sinusitis, otitis media, and the common cold. LRTIs include infection of the trachea, bronchial tubes, bronchioles, and the lungs, e.g., bronchitis and pneumonia. Symptoms of illnesses caused by coronaviruses include, e.g., fever, cough, shortness of breath, fatigue, congestion, chills, muscle pain, headache, sore throat, loss of taste or smell, diarrhea, etc.

In embodiments, the coronavirus component is a fragment of any of the proteins described herein, e.g., a structural or non-structural coronavirus protein. In embodiments, the fragment comprises a domain of the full length protein. For example, the S protein includes an N-terminal domain (S-NTD) and an ectodomain (S-ECD), which includes the spike S1 and S2 subunits. The S1 subunit also includes a receptor binding domain (S-RBD), which is responsible for binding the host receptor (e.g., ACE2 and/or NRP1). In some embodiments, the immunoassay detects a coronavirus by detecting the coronavirus S1 subunit. In some embodiments, the immunoassay detects a coronavirus by detecting the coronavirus S2 subunit. In some embodiments, the immunoassay method detects a coronavirus by detecting the coronavirus S-NTD. In some embodiments, the immunoassay method detects a coronavirus by detecting the coronavirus S-ECD. In some embodiments, the immunoassay method detects a coronavirus by detecting the coronavirus S-RBD. In embodiments, the S protein subunit (e.g., S1, S2, S-ECD, S-NTD, or S-RBD) comprises a mutation as described in Tables 1A and 1B. In embodiments, the immunoassay detects a coronavirus by detecting a combination of the coronavirus proteins described herein. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein. In embodiments, the immunoassay method detects SARS-CoV-2 by detecting SARS-CoV-2 S-D614. In embodiments, the immunoassay method detects SARS-CoV-2 by detecting SARS-CoV-2 S-D614G. In embodiments, the immunoassay method detects SARS-CoV-2 by detecting any of the SARS-CoV-2 S protein variants in Tables 1A and 1B. In embodiments, the immunoassay detects SARS-CoV-2 by detecting SARS-CoV-2 N protein and S protein.

In embodiments, the coronavirus component is a nucleic acid. As used herein in the context of viral components, a viral nucleic acid refers to a viral genome or portion thereof. The viral nucleic acid can encode a viral protein, or the viral nucleic acid can be a non-coding sequence. In embodiments, detection of a viral nucleic acid comprises detecting a sequence that is present in the viral genome, but not in the host genome. In embodiments, the coronavirus component is DNA or RNA. In embodiments, the coronavirus component comprises a nucleic acid secondary structure, e.g., an RNA loop. In embodiments, the coronavirus component is a lipid, e.g., that forms part of the viral envelope.

In embodiments, the invention provides methods for distinguishing between strains of a coronavirus. The term “strain” is used interchangeably herein with “variant,” “lineage.” and “type.” In embodiments, a mutant strain or variant of a virus described herein, e.g., SARS-CoV-2, comprises one or more mutations relative to a reference or parent or wild-type strain of the virus. As referred to throughout this application, the SARS-CoV-2 NC_045512 strain is the “reference” or “wild-type” strain, and all SNPs described herein are attributed to one or more “mutant” strains or “variants.” In embodiments, the invention provides methods to trace the lineage of a coronavirus in a population. For example, two strains of SARS-CoV-2 have been identified, referred to as the “L” strain (also known as “lineage B”) and “S” strain (also known as “lineage A”). The L strain can be differentiated from the more ancestral S strain based on two different SNPs that show nearly complete linkage: one at location 8782 (orflab: T8517C, synonymous) and one at location 28144 (ORF8: C251T, S84L). See, e.g., Tang et al., Natl Sci Rev, nwaa036; doi:10.1093/nsr/nwaa036 (3 Mar. 2020). Moreover, as discussed herein, two SARS-CoV-2 strains have been identified to contain an SNP at genome location 23403, which encodes the S protein, and are referred to herein as the “S-D614” and “S-D614G” strains. A further SARS-CoV-2 SNP of interest is at location 11083, where the 11083G to T mutation (denoted as “11083G>T”) is associated with asymptomatic presentation. In embodiments, the SARS-CoV-2 reference strain comprises the “L strain” SNP at genome locations 8782 and 28144, the “S-D614” SNP at genome location 23403, and a G nucleotide at genome location 11083.

In embodiments, SARS-CoV-2 strains are characterized by SNPs in the coding sequence of the S protein. Such SARS-CoV-2 strains include, e.g., the B.1.1.7 strain (also referred to as the “UK strain”), the 501Y.V2 strain (also known as the B.1.351 strain and referred to as the “South Africa strain”), the P.1 strain (also referred to as the “Brazil strain”), the P.2 strain, the Cal.20C strain (also known as the B.1.429 strain and referred to as the “California strain”), the B.1.525 strain (also referred to as the “Nigeria strain”), the B.1.526 strain (also referred to as the “New York strain”), and the B.1.617 strain (also referred to as the “India strain”). The B.1.1.7 strain is characterized by the following mutations in the S protein: a deletion of amino acid residues 69-70, E484K, N501Y, D614G, and P681H. The 501Y.V2 strain is characterized by the following mutations in the S protein: D215G, K417N, E484K, N501Y, and D614G. The P.1 strain is characterized by the following mutations in the S protein: K417T, E484K, N501Y, and D614G. The Cal.20C strain is characterized by a L452R mutation in the S protein. The B.1.526 strain comprises the following mutations in the S protein: L5F, T951, D253G, D614G, A701V. and either E484K or S477N. The B.1.526 strain comprising E484K is referred to herein as “B.1.526/E484K” and the B.1.526 strain comprising S477N is referred to herein as “B.1.526/S477N.” As used herein, strains “characterized” by particular mutations include at least those particular mutations and may include additional mutations. These strains and associated mutations are summarized in Table 1A. Additional variants of SARS-CoV-2 comprise mutations in the S protein as shown in Table 1B and are further described, e.g., in Faria et al., “Genomic characterisation of an emergent SARS-CoV-2 lineage in Manaus: preliminary findings” (2020). Accessed at <virological.org/t/586>; Wu et al., bioRxiv doi:10.1101/2021.01.25.427948 (2021); Guruprasad, Proteins 2021:1-8 (2021); Zhou et al., bioRxiv doi:10.1101/2021.03.24.436620 (2021). Further strains and mutations of SARS-CoV-2 are provided in the Nextstrain database (nextstrain.org); the Global Evaluation of SARS-CoV-2/hCoV-19 Sequences (GESS) database provided by Fang et al., Nucleic Acid Res 49(D1):D706-D714 (2021) (wan-bioinfo.shinyapps.io/GESS); and the SARS-CoV-2 Mutation Browser provided by Rakha et al., bioRxiv doi: 10.1101/2020.06.10.145292 (2020) (covid-19.dnageography.com). The mutations denoted as “del” or “Δ” indicate a deletion of the indicated amino acid residues.

Throughout this application, when referring to an S protein comprising a specific mutation, the mutation is relative to the SARS-CoV-2 reference strain NC_045512. The S protein from the SARS-CoV-2 reference strain is also known as the “wild-type” S protein. For example, the S-D614G protein from SARS-CoV-2 comprises D to G substitution at amino acid residue 614 relative to the wild-type S protein from SARS-CoV-2.

TABLE 1A
SARS-CoV-2 Strains and Associated Mutations

| | | | |
|---|---|---|---|
| Genome location | | Amino Acid | Exemplary |
| (based on SARS- | | Change in | Associated |
| CoV-2 reference | Nucleotide | Corresponding | SARS-CoV-2 |
| strain NC_045512) | Change | Protein | Strain(s) |

5′ UTR

| | | |
|---|---|---|
| 241 | C>T | (N/A) |

nsp3

| | | |
|---|---|---|
| 3037 | C>T | F924F |

ORF1ab

| | | | |
|---|---|---|---|
| 1059 (Orf1a) | C>T | T265I | |
| 3267 | C>T | T1001I | B.1.1.7 |
| 3828 | C>T | S1188L | P.1 |
| 5230 | G>T | K1655N | 501Y.V2 |
| 5388 | C>A | A1708D | B.1.1.7 |
| 5648 | A>C | K1795Q | P.1 |
| 6954 | T>C | I2230T | B.1.1.7 |
| 11288-11296 | deletion | SGF3675-3677 | B.1.1.7 |
| | | del | |
| 17259 | G>T | E5665D | P.1 |

RdRp

| | | |
|---|---|---|
| 14408 | C>T | P323L |

S Protein

| | | | |
|---|---|---|---|
| 21614 | C>T | L18F | P.1 |
| 21621 | C>A | T20N | P.1 |
| 21638 | C>T | P26S | P.1 |
| 21765-21770 | deletion | HV69-70 del | B.1.1.7 |
| 21801 | A>C | D80A | 501Y.V2 |
| 21974 | G>T | D138Y | P.1 |
| 21991-21993 | deletion | Y144 del | B.1.1.7 |
| 22132 | G>T | R190S | P.1 |
| 22206 | A>G | D215G | 501Y.V2 |
| 22227 | C>T | A222V | |
| 22320 | A>G | D253G | B.1.526 |
| 22812 | A>C | K417T | P.1 |
| 22813 | G>T | K417N | 507Y.V2 |
| 22865 | G>T | A435S | |
| 22917 | T>G | L452R | Cal.20C |
| 23012 | G>A | E484K | 501Y.V2, |
| | | | P.1, B.1.526 |
| 23063 | A>T | N501Y | B.1.1.7, |
| | | | 501Y.V2, P.1 |
| 73771 | C>A | A570D | B.1.1.7 |
| 23403 | A>G | D614G | B.1.1.7, |
| | | | 501Y.V2, P.1, |
| | | | B.1.429, |
| | | | B.1.526 |
| 23525 | C>T | H655Y | P.1 |
| 23604 | C>A | P681H | B.I.1.7 |
| 23664 | C>T | A701V | 501Y.V2 |
| 23709 | C>T | T716I | B.1.1.7 |
| 24506 | T>G | S982A | B.I.1.7 |
| 24642 | C>T | TI027I | P.1 |
| 24914 | G>C | D1118H | B.1.1.7 |

Orf3a

| | | |
|---|---|---|
| 25563 | G>T | Q57H |
| 26144 | G>T | G251V |

E Protein

| | | | |
|---|---|---|---|
| 26456 | C>T | P71L | 501Y.V2 |

ORF8

| | | | |
|---|---|---|---|
| 27972 | C>T | Q27stop | B.1.1.7 |
| 28048 | G>T | R52I | B.I.1.7 |
| 28111 | A>G | Y73C | B.1.1.7 |
| 28167 | G>A | E92K | P.1 |

N Protein

| | | | |
|---|---|---|---|
| 28280 | GAT>CTA | D3L | B.1.1.7 |
| 28512 | C>G | P80R | P.1 |
| 28887 | C>T | T205I | 501Y.V2 |
| 28932 | G>T | A220V | |
| 28977 | C>T | S235F | B.1.1.7 |
| 29095 | C>T | E274F | |

Orf10

| | | |
|---|---|---|
| 29645 | G>T | V30L |

TABLE 1B
Additional Mutations of the SARS-CoV-2 S Protein and Associated Strains

| S Protein Mutation(s) (relative to S | |
|---|---|
| Protein of reference strain NC_0455112) | Associated Strain |
| S Protein (e.g., S-ECD Domain) | |
| D614G | |
| D936Y | |
| A222V, D614G | |
| N439K, D614G | |
| S477N, D614G | |
| Y453F, D614G | |
| E484K, D614G | |
| N501T, D614G | |
| N501Y, D614G | |
| D936Y, D614G | |
| Q677H, D614G | |
| L18F, D80A, D215G, L242H, R246I, | B.1.351 + L18F, |
| K417N, E484K, N501Y, A701V, D614G | L242H, R2461 |
| L18F, D80A, D215G, del242-244, R246I, | B.1.351 + L18F, |
| K417N, E484K, N501Y, A701V, D614G | del242-244, R246I |
| del69-70, del144, N501Y, A570D, P681H, | B.1.1.7 |
| T716I, S982A, D1118H, D614G | |
| del69-70, del144, N501Y, A570D, | B.1.1.7 + N439K, |
| P681H, T716I, S982A, D1118H, | Y453F |
| N439K, Y4531F, D614G | |
| L18F, T20N, P26S, D138Y, | P.1 + V1176F |
| R190S, K417T, E484K, N501Y, | |
| D614G, H655Y,T1027I, V1176F | |
| SI31, W152C, L452R, D614G | B.1.429 |
| E484K, N501Y, D614G | B.1.429 − S13I |
| del69-70, del144, N501Y, A570D, P681H, | B.1.1.7 + E484K |
| T716I, S982A, D1118H, D614G, E484K | |
| D80A, D215G, K417N, E484K, | B.1.351 |
| N501Y, A701V, D614G | |
| L18F, D20N, P26S, D138Y, R190S, K417T, | P.1 |
| E484K, N501Y, D614G, H655Y, T1027I | |
| L5F, T95I, D253G, E484K, D614G, A701V | B.1.526/E484K |
| L5F, T95I, D253G, S477N, D614G, A701V | B.1.526/S477N |
| Q52R, E484K, Q677H, D614G, F888L | B.1.525 |
| Q52R, A67V, del 69-70, E484K, | B.1.525 + A67V + |
| Q677H, D614G, F888L | del69-70 |
| Q52R, A67V, del 69-70, del144, E484K, | B.1.525 + A67V + |
| Q677H, D614G, F888L | del69-70, del144 |
| E484K, D614G, V1176F | P.2 |
| del141-143, E484K, N501Y, D614G, P681H | P.3 |
| del141-143, E484K, N501Y, D614G, | P.3 + 1092K, 1101Y, |
| P681H, E1092K, F1101Y, V1176F | 1176F |
| L452R, E484Q, D614G | B.1.617 |
| E154K, L452R, E484Q, D614G, P681R | B.1.617 + E154K, |
| | 681R |
| T95I, D253G, D614G | B.1.526 reference |
| D80G, Y144del, F157S,L452R, | B.1.526.1 |
| D614G, T859N, D950H | |
| L5F, T95I, D253G, S477N, D614G, Q957R | B.1.526.2 |
| R102I (?), F157L, V367F, Q613H, P681R | A.23.1 |
| F157L, V367F, Q613H, P681R | |
| D80Y, Y144del, I210del, D215G, | |
| RSY246-248 del, L249M, W258L, R346K, | |
| T478R, E484K, H655Y, P681H, Q957H | |
| L5F, ins214TDR, Q414K, N450K, D614G, | B.1.214.2 |
| T716I, D614G, Q677H, L938F | |
| del69-70, del144, Q493R, N501Y, A570D, | BV-1 |
| D614G, P681H, T716I, S982A, D1118H | |
| T478K, D614G, P681H, T732A | BV-2 |
| S-RBD Domain | |
| N501Y | B.1.1.7 |
| K417N, E484K, N501Y | B.1.351 |
| K417T, E484K, N501Y | P.1 |
| E484K, N501Y | |
| L452R | B.1.429 |
| E484K | B.1.525 |
| N439K | |
| K417N | |
| I472V | |
| Y453F | |
| S477N | |
| S477N, E484K | |
| N501T | |
| L452R, E484Q | B.1.617 |
| Q414K, N450K | |
| R346K, T478R, E484K | |
| V367F | A.23.1 |
| K417T | |
| Q493R, 501Y | BV-1 |
| T478K | BV-2 |
| S1 subunit | |
| del69-70, del144, N501Y, A570D, D614G, | B.1.1.7 |
| P681H, K417N, E484K, N501Y, D614G | |
| D614G | |
| Q6771H | |
| Q677P | |

Further SARS-CoV-2 SNPs have been identified, for example, at the genome locations listed in Table 1C, e.g., locations 3036, 878218060, 11083, 1397, 2891, 14408, 17746, 17857, 23403, 26143, 28144, and 28881. See, e.g., Pachetti et al. J Transl Med 18:179 (2020): Banedjee et al., bioRxiv, doi.org/10.1101/2020.04.06.027854 (9 Apr. 2020); Alouane et al., bioRxiv doi.org/10.1101/2020.06.20.163188 (21 Jun. 2020); Brufsky, J Med Virol 2020:1-5 (2020), and Mishra et al., bioRxiv doi.org/10.1101/2020.05.07.082768 (12 May 2020). The ability to determine viral strain and/or trace viral lineage in a population provides valuable epidemiological insight into the spread and evolution of the virus. Determining the particular viral strain that has infected a patient also allows more comprehensive treatment. For example, the patient can be treated with a strain-specific drug. If a particular strain is more transmissible and/or more likely to cause severe illness, early interventions can be provided to the patient.

TABLE 1C
SARS-CoV-2 Single Nucleotide Polymorphisms

| Genome location | | |
|---|---|---|
| (based on SARS- | | |
| CoV-2 reference | Nucleotide | Amino |
| strain NC_045512) | Change | Acid Change |
| 241 | C > T | 5′ UTR |
| 1059 | C > T | T > I |
| 1604 | AATG > A | delTGA |
| 3037 | C > T | Synonymous |
| 4402 | T > C | Synonymous |
| 5062 | G > T | L > F |
| 8782 | C > T | Synonymous |
| 11083 | G > T | L > F |
| 11916 | C > T | S > L |
| 14408 | C > T | P > L |
| 14805 | C > T | Synonymous |
| 15324 | C > T | T > I |
| 17247 | T > C | Synonymous |
| 17747 | C > T | P > L |
| 17858 | A > G | M > V |
| 18060 | C > T | S > F |
| 18877 | C > T | H > Y |
| 22277 | C > A | Q > K |
| 22661 | G > T | V > F |
| 23403 | A > G | D > G |
| 25563 | G > T | Q > V |
| 26144 | G > T | G > V |
| 27046 | C > T | T > M |
| 27964 | C > T | S > L |
| 28144 | T > C | L > S |
| 28311 | C > T | P > L |
| 28881-28883 | GGG > AAC | R > K, G > R |
| 29540 | G > A | Upstream |
| 29553 | G > A | Intergenic |
| 29711 | G > T | Downstream |

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus nucleic acid is RNA. In embodiments, the coronavirus is SARS-CoV-2. In embodiments, the binding reagent comprises an oligonucleotide comprising a sequence complementary to the coronavirus nucleic acid sequence. In embodiments, the binding reagent binds to a nucleic acid from a specific strain of the coronavirus, e.g., the L strain or S strain of SARS-CoV-2, or the S-D614 or S-D614G strain of SARS-CoV-2, or the B.1.1.7 strain, 501Y.V2 strain, P.1 strain, or Cal.20C strain of SARS-CoV-2. In embodiments, the binding reagent binds to a SARS-CoV-2 nucleic acid encoding the N protein (i.e., the N gene). The SARS-CoV-2 N gene can be detected at three different regions: N1, N2, and N3. The N1 and N2 regions are specific to SARS-CoV-2, and the N3 region is universal to the coronaviruses in the same clade as SARS-CoV-2 (e.g., clade 2 and 3 viruses within the subgenus Sarbecovirus, including SARS-CoV-2. SARS-CoV, and bat- and civet-SARS-like CoVs. See, e.g., Lu et al., Emerg Infect Dis 26(8):1654-1665 (2020)). In embodiments, the binding reagent binds to SARS-CoV-2 N1 region, N2 region, N3 region, or a combination thereof. In embodiments, the biological sample is saliva, the coronavirus is SARS-CoV-2 and the nucleic acid is RNA.

In embodiments, the coronavirus is capable of infecting a human. In embodiments, the coronavirus causes a respiratory tract infection in a human. In embodiments, the coronavirus is SARS-CoV, MERS-CoV. SARS-CoV-2, HCoV-OC43. HCoV-229E, HCoV-NL63, HCoV-HKU1, or a combination thereof. In embodiments, the method detects a coronavirus component that is substantially conserved in SARS-CoV, MERS-CoV, SARS-CoV-2, HCoV-OC43, HCoV-229E, HCoV-NL63, and HCoV-HKU1. In embodiments, the method detects a protein or peptide fragment that is substantially conserved in SARS-CoV, MERS-CoV, SARS-CoV-2, HCoV-OC43, HCoV-229E, HCoV-NL63, and HCoV-HKU1.

In embodiments, the immunoassay described herein is a multiplexed immunoassay method. A multiplexed immunoassay can simultaneously detect multiple substances of interest, e.g., coronavirus components, in a sample. A multiplexed immunoassay can also use multiple binding reagents that specifically bind a substance of interest, e.g., a coronavirus component, in a sample. Multiplexed immunoassays can provide reliable results while reducing processing time and cost. In embodiments, a multiplexed immunoassay for detecting a coronavirus comprises multiple binding reagents, each of which binds to a different coronavirus component, e.g., a conserved coronavirus protein. In embodiments, a multiplexed immunoassay comprising binding reagents that each specifically binds a different coronavirus component provides improved detection accuracy, e.g., over a singleplex method utilizing a single binding reagent. In embodiments, the immunoassay method detects a coronavirus by detecting one or more of the coronavirus E protein, S protein, including S1 and S2 subunits, S-NTD, S-ECD, and S-RBD. M protein, HE protein, N protein, nsp1, nsp2, nsp3, nsp4, nsp5, nsp6, nsp7, nsp8, nsp9, nsp10, nsp11, nsp12, nsp13, nsp14, nsp15, and nsp16. In embodiments, the coronavirus is SARS-CoV-2. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein. In embodiments, the immunoassay method detects SARS-CoV-2 by detecting SARS-CoV-2 S-D614. In embodiments, the immunoassay method detects SARS-CoV-2 by detecting SARS-CoV-2 S-D614G. In embodiments, the immunoassay method detects SARS-CoV-2 by detecting any of the SARS-CoV-2 S protein variants in Tables 1A and 1B. In embodiments, the immunoassay detects SARS-CoV-2 by detecting SARS-CoV-2 N protein and S protein. In embodiments, the immunoassay detects SARS-CoV-2 by detecting any combination of the SARS-CoV-2 N protein, S protein, E protein, and M protein. In embodiments, the immunoassay detects SARS-CoV-2 by detecting SARS-CoV-2 N protein, S protein, E protein, and M protein. In embodiments, the immunoassay detects SARS-CoV-2 by detecting any of the SARS-CoV-2 protein variants in Table 1A.

In embodiments, the immunoassay method is a multiplexed method comprising: contacting the biological sample with a surface comprising a binding reagent in each binding domain on the surface, wherein the binding reagent in each binding domain independently binds to a viral protein selected from SARS-CoV-2 N protein, SARS-CoV-2 S protein, SARS-CoV-2 E protein, SARS-CoV-2 M protein, or a combination thereof; forming a binding complex in each binding domain comprising the viral protein and the binding reagent that binds to the viral protein; and measuring the concentration of the viral protein in each binding complex. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises any of the mutations shown in Tables 1A and 1B. In embodiments, each binding complex further comprises a detection reagent that specifically binds to the viral protein of the binding complex. Detection reagents are further described herein.

In embodiments, the immunoassay method is a multiplexed method capable of simultaneously detecting multiple coronaviruses in a biological sample. In embodiments, the multiplexed method is capable of simultaneously detecting one or more of SARS-CoV, MERS-CoV, SARS-CoV-2, HCoV-OC43, HCoV-229E, HCoV-NL63, and HCoV-HKU1.

In embodiments, the binding reagent and/or the detection reagent that specifically binds to the coronavirus component described herein is an antibody, antigen, ligand, receptor, oligonucleotide, hapten, epitope, mimotope, or aptamer. In embodiments, the binding reagent and/or the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent and/or the detection reagent comprises at least one heavy or light chain complementarity determining region (CDR) of an antibody. In embodiments, the binding reagent and/or the detection reagent comprises at least two CDRs from one or more antibodies. In embodiments, the binding reagent and/or the detection reagent is an antibody or antigen-binding fragment thereof. In embodiments, the binding reagent and/or the detection reagent is a receptor for the coronavirus component. In embodiments, the binding reagent and/or the detection reagent is a receptor for the coronavirus S protein. In embodiments, the binding reagent and/or the detection reagent is angiotensin-converting enzyme 2 (ACE2). In embodiments, the binding reagent and/or the detection reagent is neuropilin-1 (NRP1). In embodiments, the binding reagent and/or the detection reagent is CD147.

In embodiments where the method comprises detecting one or more variants of an SARS-CoV-2 protein (e.g., an S protein comprising a mutation shown in Tables 1A and 13 or an Orf1ab, E, Orf8, or N protein comprising a mutation shown in Table 1A), the binding reagent comprises an antibody or antigen-binding fragment thereof that is capable of specifically binding the wild-type, protein variant(s), or both the protein variant and the wild-type, and the detection reagent comprises an antibody or antigen-binding fragment thereof that is capable of binding the wild-type, protein variant(s), or both the wild-type and variant forms of the protein. In embodiments, the SARS-CoV-2 protein is an S protein, an N protein, an E protein, an Orf1ab protein, an Orf8 protein, or a combination thereof. In embodiments, the SARS-CoV-2 protein is an S protein.

In embodiments, the method is capable of detecting about 1 fg/mL to about 1 ng/mL, about 1 fg/mL to about 0.8 ng/mL, about 1 fg/mL to about 0.5 ng/mL, about 1 fg/mL to about 0.1 ng/mL, about 1 fg/mL to about 50 pg/mL, about 1 fg/mL to about 20 pg/mL, about 1 fg/mL to about 10 pg/mL, about 1 fg/mL to about 5 pg/mL, about 1 fg/mL to about 2 pg/mL, about 1 fg/mL to about 1 pg/mL, about 5 fg/mL to about 100 fg/mL, about 7 fg/mL to about 75 fg/mL, or about 10 fg/mL to about 50 fg/mL of a virus (e.g., a coronavirus such as SARS-CoV-2). In embodiments, the method is capable of detecting less than or about 5 pg/mL, less than or about 2 pg/mL, less than or about 1 pg/mL, less than or about 500 fg/mL, less than or about 100 fg/mL, less than or about 75 fg/mL, less than or about 50 fg/mL, or less than or about 10 fg/mL of a virus (e.g., a coronavirus such as SARS-CoV-2). In embodiments, the method is capable of detecting less than or about 10⁹ viral particles per mL, less than or about 10⁸ viral particles per mL, less than or about 107 viral particles per mL, less than or about 106 viral particles per mL, less than or about 100000 viral particles per mL, less than or about 10000 viral particles per mL, less than or about 1000 viral particles per mL, or less than or about 100 viral particles per mL. In embodiments where the method detects a viral nucleic acid, one viral particle is one viral genome equivalent. In embodiments, the method is capable of detecting less than or about 10⁹ viral genome equivalents per mL, less than or about 10⁸ viral genome equivalents per mL, less than or about 10⁷ viral genome equivalents per mL, less than or about 10⁶ viral genome equivalents per mL, less than or about 100000 viral genome equivalents per mL, less than or about 10000 viral genome equivalents per mL, less than or about 1000 viral genome equivalents per mL, or less than or about 100 viral genome equivalents per mL.

### Biomarkers

In embodiments, the invention provides a method for detecting a biomarker that is produced by a host (e.g., a human subject) in response to a viral infection, e.g., by a respiratory virus, including coronaviruses such as SARS-CoV-2. As used herein, “host” refers to a subject who has been infected with or suspected of being infected with a virus described herein, e.g., a coronavirus such as SARS-CoV-2. Unless otherwise specified, the biomarkers described herein are produced by a host, e.g., a human subject, in response to viral exposure and/or infection as described herein. In embodiments, the biomarker is an immune response biomarker. In embodiments, the biomarker is an antibody. In embodiments, the biomarker is an inflammation response biomarker. In embodiments, the biomarker is a damage response biomarker. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is a coronavirus. In embodiments, the virus is SARS-CoV-2.

As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility or responsiveness of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2): 182-188 (2004); Strimbu et al., Curr Opin HIV AIDS 5(6): 463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. Unless specified otherwise, the biomarkers referred to herein encompass human biomarkers.

As used herein, the term “level” in the context of a biomarker refers to the amount, concentration, or activity of a biomarker. The term “level” can also refer to the rate of change of the amount, concentration, or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide (e.g., an antibody), nucleic acid, or small molecule. “Level” can also refer to an absolute amount of a biomarker in a sample or to a relative amount of the biomarker, including amount or concentration determined under steady-state or non-steady-state conditions. “Level” can further refer to an assay signal that correlates with the amount, concentration, activity or rate of change of a biomarker. The level of a biomarker can be determined relative to a control marker in a sample.

Measurement of biomarker values and levels before and after a particular event, e.g., cellular or environmental event, may be used to gain information regarding an individual's response to the event. For example, samples or model organisms can be subjected to stress- or disease-inducing conditions, or a treatment or prevention regimen, and a particular biomarker can then be detected and quantitated in order to determine its changes in response to the condition or regimen. However, the opposite, i.e., measuring biomarker values and levels to determine whether an organism has been subjected to stress- or disease-inducing condition, tends to be much more complicated, as changes in the levels of a single biomarker are sometimes not definitively associated with a particular condition.

In embodiments, the measured levels of the one or more biomarkers described herein provides information regarding infection and immune response to infection, e.g., the course or maturity of infection, the etiology of severe illness, and the potential severity of illness. In embodiments, the measured levels of the one or more biomarkers described herein provides information regarding a subject's antibody response, cytokine response, neutrophil, macrophage, and/or monocyte production, complement activation, B cell and/or T cell activation, or a combination thereof.

In embodiments, detection and/or measurement of a single biomarker is sufficient to provide a prediction and/or diagnosis of a disease or condition. In embodiments, combinations of biomarkers are used to provide a strong prediction and/or diagnosis. Although a linear combination of biomarkers (i.e., the combination comprises biomarkers that individually provide a relatively strong correlation) can be utilized, linear combinations may not be available in many situations, for example, when there are not enough biomarkers available and/or with strong correlation. In alternative approaches, a biomarker combination is selected such that the combination is capable of achieving improved performance (i.e., prediction or diagnosis) compared with any of the individual biomarkers, each of which may not be a strong correlator on its own. Biomarkers for inclusion in a biomarker combination can be selected for based on their performance in different individuals, e.g., patients, wherein the same biomarker may not have the same performance in different individuals, but when combined with the remaining biomarkers, provide an unexpectedly strong correlation for prediction or diagnosis in a population. For example, Bansal et al., Statist Med 32: 1877-1892 (2013) describe methods of determining biomarkers to include in such a combination, noting in particular that optimal combinations may not be obvious to one of skill in the art, especially when subgroups are present or when individual biomarker correlations are different between cases and controls. Thus, selecting a combination of biomarkers for providing a consistent and accurate prediction and/or diagnosis can be particularly challenging and unpredictable.

Even when a suitable combination of biomarkers is determined, utilizing the combination of biomarkers in an assay poses its own set of difficulties. For example, detecting and/or quantitating each biomarker in the combination in its own separate assay may not be feasible with small samples, and using a separate assay to measure each biomarker in a sample may not provide consistent and comparable results. Furthermore, running an individual assay for each biomarker in a combination can be a cumbersome and complex process that can be inefficient and costly.

A multiplexed assay that can simultaneously measure the concentrations of multiple biomarkers can provide reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive, all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output measurement.

In embodiments, the invention provides methods of assessing an individual's immune response to a viral infection. In embodiments, the invention provides methods of assessing a group of individuals immune response to a viral infection. In embodiments, assessing an immune response comprises determining the type and/or strength of the immune response, e.g., detecting the molecular components produced in response to a viral infection (e.g., acute phase reactants, antibodies, cytokines, etc.) and measuring the amounts of each component produced. In embodiments, the invention provides methods of assessing the differences in immune responses by age, race, ethnicity, socioeconomic backgrounds, and/or underlying conditions, e.g., lung disease, diabetes, cancer, etc., which may be associated with poor clinical outcomes. In embodiments, the invention provides methods of determining the epidemiology of diseases caused by the viruses described herein, e.g., COVID-19. In embodiments, the virus is a coronavirus. In embodiments, the virus is SARS-CoV-2.

In embodiments, the invention provides methods of assessing cross-reactivity of an individual's immune response between different coronaviruses (e.g., SARS-CoV, MERS-CoV, SARS-CoV-2, HCoV-OC43, HCoV-229E, HCoV-NL63, and HCoV-HKU1). In embodiments, the invention provides methods of mapping the epitopes recognized by an individual's immune response, e.g., epitopes on a coronavirus S protein. In embodiments, the invention provides methods of assessing the individual's clinical outcome based on the mapped epitopes of immune responses. In embodiments, the invention provides methods of assessing an individual's immune response by detecting different IgG classes and/or subclasses. In embodiments, the invention provides methods of assessing the individual's clinical outcome based on the IgG classes and/or subclasses. In embodiments, the invention provides methods of assessing the affinity and/or avidity of an individual's immune response to different viral antigens. In embodiments, the invention provides methods of assessing the strength of an immune response. e.g., measuring the total antibody concentration or the concentration of different classes or subclasses of antibodies in an individual. In embodiments, the invention provides methods of determining the natural interacting partner(s) of the virus, e.g., a coronavirus such as SARS-CoV-2. As used in the context of viral infections, a “natural interacting partner” refers to a substance in the host cell (e.g., proteins or carbohydrate moieties on a host cell surface) that interacts with a viral component described herein. Natural interacting partners of viruses are further described in, e.g., Brito et al., Front Microbiol 8:1557 (2017). Natural interacting partners of SARS-CoV-2 include, e.g., ACE2, NRP1, and CD147, and are further described in Gordon et al., bioRxiv 2020.03.22.002386v1 (2020) doi:10.1101/2020.03.22.002386v1, Daly et al., bioRxiv 2020.06.05. 134114 (2020) doi:10.1101/2020.06.05.134114, and Bojkova et al., Nature Research (Pre-Print 11 Mar. 2020) doi:10.21203/rs.3.rs-17218/v1. In embodiments, the invention provides a competitive assay for SARS-CoV-2 utilizes ACE2, NRP1, CD147, or different sialic acid-containing substances to determine the interacting partner(s) of the SARS-CoV-2 S protein.

In embodiments, the invention provides methods of assessing changes in the immune response over time. In embodiments, the invention provides methods of assessing an individual's immune response at different time points after infection and/or after the first onset of a symptom. In embodiments, the invention provides methods of assessing the cytokines present in an individual at different time points after infection and/or after the first onset of a symptom. Symptoms of viral infections are described herein. In embodiments, the invention provides methods of assessing the long-term effects of an infection on an individual. For example, the coronavirus SARS-CoV-2 can cause post-acute COVID-19 syndrome (also known as post-COVID syndrome or “long COVID”), in which symptoms of the infection, including fatigue, headaches, shortness of breath, anosmia, muscle weakness, low fever, and cognitive dysfunction, persist for weeks or months after the typical convalescence period of COVID-19. In embodiments, the invention provides methods of assessing an individual's immune response at different time points after vaccination. In embodiments, the invention provides methods of determining the immune response components that provide immunity to a viral infection. In embodiments, the invention provides methods of assessing an individual's immune response at different time points after receiving a treatment for the viral infection. In embodiments, the invention provides methods of assessing the effect of convalescent serum treatment in an individual, e.g., comprising measuring the individual's immune response after administration of the convalescent serum. In embodiments, the invention provides methods of assessing the immune response components (e.g., antibodies) present in a convalescent serum sample. e.g., comprising determining its effectiveness, half life, and/or functional window of treatment in an individual. In embodiments, the invention provides methods of assessing the effectiveness, half life, and/or functional window of protection of a therapeutic antibody treatment. In embodiments, the virus is a coronavirus. In embodiments, the virus is SARS-CoV-2.

In embodiments, the invention provides methods of assessing an individual's immune response, e.g., an antibody, to a coronavirus (e.g., an endemic coronavirus such as HCoV-OC43, HCoV-229E, HCoV-NL63, and HCoV-HKU) to determine a clinical outcome of infection by a different coronavirus, e.g., SARS-CoV-2. In embodiments, the invention provides methods of assessing an individual's immune response, e.g., an antibody, to a respiratory virus (e.g., influenza or RSV) to determine a clinical outcome of infection by a different respiratory virus, e.g., SARS-CoV-2.

Serology tests that assess the presence of an antibody biomarker against a SARS-CoV-2 antigen have received U.S. FDA Emergency Use Authorization (EUA) with specificity of 95%. In embodiments, the invention provides improved sensitivity and/or specificity in determining whether a subject is currently infected or has previously been infected with a virus, e.g., a coronavirus such as SARS-CoV-2. In embodiments, the invention provides improved sensitivity and/or specificity in determining whether a subject has immunity to a virus, e.g., a coronavirus such as SARS-CoV-2. In embodiments, the methods herein have a sensitivity of greater than 90%, greater than 95%, greater than 96%, greater than 97%, greater than 98%, greater than 99%, greater than 99.5%, or greater than 99.9%. In embodiments, the methods herein have a specificity of greater than 90%, greater than 95%, greater than 96%, greater than 97%, greater than 98%, greater than 99%, greater than 99.5%, or greater than 99.9%. Assays with high sensitivity and specificity are important to correctly diagnose active infections and to correctly determine whether an individual has been previously exposed and/or immune to a virus, e.g., a coronavirus such as SARS-CoV-2. In particular, assays with high specificity are useful for conducting epidemiological studies in populations with low disease prevalence. Moreover, assays with high specificity are important for individual assessment due to the high risk of a false positive to the individual and the individual's community; individuals who received a false positive serology test result for SARS-CoV-2 may believe themselves to be immune and therefore erroneously engage in activity that can increase the likelihood of infection and spread of the virus.

Antibody Biomarkers

In embodiments, the invention provides a method for detecting a respiratory virus, e.g., a coronavirus such as SARS-CoV-2, in a biological sample, by detecting a biomarker produced in response to an infection by the virus. In embodiments, the biomarker produced in response to a viral infection is an antibody.

In embodiments, the invention provides a method for detecting a biomarker that is capable of binding to a viral antigen in a biological sample. As used herein, a virus or viral antigen is any component or secretion of a virus that prompts an immune response in a host (e.g., a human). In embodiments, the viral antigen is a viral protein or fragment thereof. In embodiments, the viral antigen is a virus structural protein. In embodiments, the viral antigen is a virus nonstructural protein. Structural and nonstructural proteins of viruses, e.g., respiratory viruses such as coronaviruses, are described herein. In embodiments, the method is capable of determining whether a subject has been exposed to a particular virus, e.g., a coronavirus such as SARS-CoV-2. In embodiments, the method is capable of determining whether a subject is at risk of being infected by a particular virus, e.g., a coronavirus such as SARS-CoV-2. In embodiments, the method is capable of determining whether a subject has immunity to a particular virus. e.g., a coronavirus such as SARS-CoV-2.

In embodiments, the invention provides an immunoassay method comprising: quantifying the amounts of one or more biomarkers capable of binding to a respiratory virus antigen in a biological sample, wherein the respiratory virus is a coronavirus, an influenza virus, a paramyxovirus, an adenovirus, a bocavirus, a pneumovirus, an enterovirus, a rhinovirus, or a combination thereof, wherein the quantifying comprises measuring the concentrations of each of the one or more biomarkers in an immunoassay.

In embodiments, the immunoassay method comprises: contacting the biological sample with a surface comprising a viral antigen in a binding domain on the surface; forming a binding complex in the binding domain comprising the viral antigen and a biomarker that binds to the viral antigen; and measuring the concentration of the biomarker in the binding complex. In embodiments, the biomarker is IgG, IgA, IgM, or combination thereof. In embodiments, the concentration of the biomarker is measured by contacting the binding complex with a detection reagent that specifically binds IgG, IgA, or IgM. In embodiments, the biomarker is a human biomarker, a mouse biomarker, a rat biomarker, a ferret biomarker, a minx biomarker, a bat biomarker, or a combination thereof. In embodiments, the biomarker is human IgG, IgA, or IgM. In embodiments, the biomarker is mouse IgG, IgA, or IgM. In embodiments, the biomarker is rat IgG. IgA, or IgM. In embodiments, the biomarker is ferret IgG, IgA, or IgM. In embodiments, the biomarker is minx IgG, IgA, or IgM. In embodiments, the biomarker is bat IgG, IgA, or IgM. Detection reagents are further described herein.

Respiratory viruses and proteins thereof are further described herein. In embodiments, the immunoassay method is capable of detecting a coronavirus, an influenza virus, a respiratory syncytial virus (RSV), or a combination thereof. In embodiments, the immunoassay method detects a biomarker that binds to a viral antigen from SARS-CoV-2, SARS-CoV, MERS-CoV, HCoV-OC43, HCoV-229E, HCoV-NL63, HCoV-HKU1, influenza A, influenza B, RSV, or a combination thereof. In embodiments, the viral antigen comprises nucleocapsid protein (N) from SARS-CoV-2, N protein from SARS-CoV. N protein from MERS-CoV, N protein from HCoV-229E, N protein from HCoV-NL63, N protein from HCoV-HKU1, N protein from HCoV-OC43, spike protein (S) from SARS-CoV-2, S protein from SARS-CoV, S protein from MERS-CoV, S protein from HCoV-229E, S protein from HCoV-NL63, S protein from HCoV-HKU1, S protein from HCoV-OC43, hemagglutinin (HA) from influenza B strain, influenza A H1 strain (e.g., H1/Michigan strain), influenza A H3 strain (e.g., H3/Hong Kong strain), influenza A H7 strain (e.g., H7/Shanghai strain), fusion protein (F), including, e.g., pre-fusion and post-fusion variants, from respiratory syncytial virus (RSV), or a combination thereof. In some embodiments, the S protein is a subunit, domain, or fragment thereof, e.g., S1, S2, S-NTD, S-ECD, or S-RBD as described herein. In some embodiments, the S protein is SARS-CoV-2 S-D614. In some embodiments, the S protein is SARS-CoV-2 S-D614G. In embodiments, the S protein is a SARS-CoV-2 S protein or subunit or fragment thereof that comprises any of the mutations shown in Tables 1A and 1B. In embodiments, the N protein is a SARS-CoV-2 N protein that comprises any of the mutations shown in Table 1A.

In embodiments, the immunoassay method detects a biomarker that binds to an N protein from SARS-CoV-2. In embodiments, the immunoassay method detects a biomarker that binds to a S protein from SARS-CoV-2. In embodiments, the immunoassay method detects a biomarker that binds to S1, S2, S-ECD, S-NTD, or S-RBD from SARS-CoV-2. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A. In embodiments, the immunoassay method comprises, contacting the biological sample with a surface comprising a viral antigen in a binding domain on the surface; forming a binding complex in the binding domain comprising the viral antigen and a biomarker that binds to the viral antigen, and measuring the concentration of the biomarker in the binding complex. In embodiments, the biomarker is IgG, IgA, IgM, or combination thereof. In embodiments, the biomarker is an IgG, IgA, and/or IgM from a human, mouse, rat, ferret, minx, bat, or combination thereof. In embodiments, the concentration of the biomarker is measured by contacting the binding complex with a detection reagent that specifically binds IgG, IgA, or IgM as described herein. In embodiments, the immunoassay method is a classical serology assay. In embodiments, the immunoassay method is a bridging serology assay. In embodiments, the immunoassay method is a competitive serology assay. In embodiments, the detection reagent comprises a labeled competitor of the biomarker. In embodiments, the competitor is ACE2. Classical, bridging, and competitive serology assays are described herein.

In embodiments, the method is a multiplexed method capable of simultaneously detecting and/or quantifying the amounts of the one or more biomarkers that bind to a respiratory virus antigen. As discussed herein, a method that is capable of simultaneously testing for several potential causes of infection (e.g., multiple different viruses) can advantageously allow a respiratory virus infection to be correctly and efficiently diagnosed in a single assay run and utilizing a single patient sample. Such as method can also be useful for assessing a patient's immune response to different respiratory virus infections.

In embodiments, the multiplexed method simultaneously detects and/or quantifies one or more biomarkers that binds to: an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, and/or an F protein from RSV. In embodiments, the S protein is a subunit, domain, or fragment thereof. e.g., S1, S2, S-NTD, S-ECD, or S-RBD. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S protein from SARS-CoV-2, an S-RBD from SARS-CoV-2, an S-NTD from SARS-CoV-2, an S protein from SARS-CoV, an S-protein from MERS-CoV, an S protein from HCoV-HKU1, an S protein from HcoV-OC43, an HA from influenza strain B/Brisbane, an HA from influenza strain B/Phuket, an HA from influenza strain H1/Michigan, an HA from influenza strain H3/Hong Kong, and/or an HA from influenza strain H7/Shanghai. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, and/or an S protein from SARS-CoV. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, and an S protein from SARS-CoV. In embodiments, the multiplexed method simultaneously detects and/or quantifies one or more biomarkers that binds to: an N protein from SARS-CoV-2, an S-RBD from SARS-CoV-2, and/or an S protein from SARS-CoV-2. In embodiments, the multiplexed method simultaneously detects and/or quantifies one or more biomarkers that binds to: an N protein from SARS-CoV-2, an S-RBD from SARS-CoV-2, and an S protein from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to S proteins from different strains SARS-CoV-2. For example, as described herein, the S protein of SARS-CoV-2 strain B.1.1.7 (“UK”) comprises a deletion of residues 69-70, and the substitutions N501Y, D614G, and P681H. The S protein of SARS-CoV-2 strain 501Y.V2 (“South Africa”) strain comprises the substitutions D215G, K417N, E484K, N501Y, D614G, and A701V. The S protein of SARS-CoV-2 strain P.1 (“Brazil”) comprises the substitutions R190S, K417T, E484K, N501Y, and D614G. The S protein of SARS-CoV-2 strain Cal.20C (“California”) strain comprises the substitution L452R. As discussed herein, when referring to an S protein comprising a specific mutation, the mutation is relative to the SARS-CoV-2 reference strain NC_045512, and the S protein from the SARS-CoV-2 reference strain is also known as the “wild-type” S protein. Moreover, an S protein (or subunit thereof) referred to herein as being from a specific SARS-CoV-2 strain includes all of the S protein mutations of that strain as described herein. Unless otherwise specified, the SARS-CoV-2 strains B.1.1.7, 501Y.V2, P.1, and Cal.20C do not comprise mutations in the N protein, envelope protein, membrane protein, or other nonstructural proteins (e.g., Orf7a, Orf8) relative to the reference strain.

In embodiments, the multiplexed method simultaneously detects and/or quantifies one or more biomarkers that binds to an S protein or subunit thereof from the SARS-CoV-2 reference strain NC_045512; an S protein or subunit thereof from the SARS-CoV-2 B.1.1.7 strain; an S protein or subunit thereof from the SARS-CoV-2501Y.V2 strain, the SARS-CoV-2; an S protein or subunit thereof from the SARS-CoV-2 P1 strain; and an S protein or subunit thereof from the SARS-CoV-2 Cal.20C strain. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a wild-type S protein from SARS-CoV-2, an S-D614G from SARS-CoV-2, an S protein from SARS-CoV-2 strain B.1.1.7, an S protein from SARS-CoV-2 strain 501Y.V2, an S protein from SARS-CoV-2 strain P.1, an S protein from SARS-CoV-2 strain Cal.20C, a wild-type S-RBD from SARS-CoV-2, an S-RBD from SARS-CoV-2 strain B.1.1.7, an S-RBD from SARS-CoV-2 strain 501Y.V2, an S-RBD from SARS-CoV-2 strain P.1, an S-RBD from SARS-CoV-2 strain Cal.20C, a wild-type S-NTD from SARS-CoV-2, an N protein from SARS-CoV-2, an Orf8 protein (monomeric or oligomeric form) from SARS-CoV-2, an Orf7a protein from SARS-CoV-2, a membrane (Mem) protein from SARS-CoV-2, and/or an envelope (Env) protein from SARS-CoV-2. In embodiments, the one or more biomarkers is IgG, IgA, IgM, or combination thereof. In embodiments, the IgG. IgA, and/or IgM is from a human, mouse, rat, ferret, minx, bat, or combination thereof.

In embodiments, the multiplexed method simultaneously detects and/or quantifies one or more biomarkers that binds to: a wild-type S protein from SARS-CoV-2, an N protein from SARS-CoV-2, an S-RBD from SARS-CoV-2 strain 501Y.V2, and an S protein from SARS-CoV-2 strain 501Y.V2. In embodiments, the multiplexed method simultaneously detects and/or quantifies one or more biomarkers that binds to: a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the multiplexed method simultaneously detects and/or quantifies one or more biomarkers that binds to: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the multiplexed method simultaneously detects and/or quantifies one or more biomarkers that binds to: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the multiplexed method simultaneously detects and/or quantifies one or more biomarkers that binds to: a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the multiplexed method simultaneously detects and/or quantifies one or more biomarkers that binds to: a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and/or an HA from influenza A H3. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and an HA from influenza A/Hong Kong H3. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A H3, an HA from influenza A H1, an HA from influenza A H7, an HA from influenza B/Phuket, and/or an HA from influenza B/Brisbane. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A H3, an HA from influenza A H1, an HA from influenza A H7, an HA from influenza B/Phuket, an HA from influenza B/Brisbane, and/or an F protein from RSV. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and/or an S protein from HCoV-229E. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and an S protein from HCoV-229E. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A H3, an HA from influenza A H1, an HA from influenza A H7, an HA from influenza B/Phuket, an HA from influenza B/Brisbane, and/or an F protein from RSV. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and/or an S protein from HCoV-229E. In embodiments, the multiplexed method simultaneously detects and/or quantifies one or more biomarkers that binds to: an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and an S protein from HCoV-229E. In embodiments, the multiplexed method simultaneously detects and/or quantifies one or more biomarkers that binds to: an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A H3, an HA from influenza A H1, an HA from influenza A H7, an HA from influenza B/Phuket, an HA from influenza B/Brisbane, and/or an F protein from RSV. In embodiments, the multiplexed method simultaneously detects and/or quantifies one or more biomarkers that binds to: an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza/Shanghai H7, an HA from influenza B/Phuket, an HA from influenza B/Brisbane, and an F protein from RSV. In embodiments, the one or more biomarkers is IgG. IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S-RBD from SARS-CoV-2, an S protein from SARS-CoV-2, an S2 from SARS-CoV-2, an S protein from SARS-CoV, an S protein from HCoV-OC43, an S protein from HCoV-HKU1, an S protein from HCoV-NL63, and/or an S protein from HCoV-229E. In embodiments, the multiplexed method simultaneously detects and/or quantifies one or more biomarkers that binds to: an N protein from SARS-CoV-2, an S-RBD from SARS-CoV-2, an S protein from SARS-CoV-2, an S2 from SARS-CoV-2, an S protein from SARS-CoV, an S2 from SARS-CoV-2, an S protein from HCoV-OC43, an S protein from HCoV-HKU1, an S protein from HCoV-NL63, and an S protein from HCoV-229E. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and/or an S-RBD from SARS-CoV-2. In embodiments, the multiplexed method simultaneously detects and/or quantifies one or more biomarkers that binds to: an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and/or an S-RBD from SARS-CoV-2. In embodiments, the multiplexed method simultaneously detects and/or quantifies one or more biomarkers that binds to: an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an S protein from SARS-CoV-2, an S-RBD from SARS-CoV-2, an S-NTD from SARS-CoV-2, an N protein from SARS-CoV-2, an S protein from SARS-CoV, and/or an S protein from MERS-CoV. In embodiments, the multiplexed method simultaneously detects and/or quantifies one or more biomarkers that binds to: an HA protein from FluB/Brisbane/60/2008, an HA protein from FluB/Phuket/3073/2013, an HA protein from FluA/Michigan/45/2015 (H1N1), an HA protein from FluA/HongKong/4801/2014 (H3N2), an HA protein from FluA/Shanghai/2/2013 (H7N9), an S protein from HCoV-HKU1, and/or an S protein from HCoV-OC43. In embodiments, the multiplexed method simultaneously detects and/or quantifies one or more biomarkers that binds to: an S protein from SARS-CoV-2, an S-RBD from SARS-CoV-2, an S-NTD from SARS-CoV-2, an N protein from SARS-CoV-2, an S protein from SARS-CoV, an S protein from MERS-CoV, an S protein from HCoV-HKU1, an S protein from HCoV-OC43, and/or an HA protein from FluA/HongKong/4801/2014 (H3N2). In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an HA protein from influenza A H3, an HA protein from influenza A H1, an HA protein from influenza A H7, an HA protein from influenza B/Phuket; and/or an HA protein from influenza B/Brisbane. In embodiments, the multiplexed method simultaneously detects and/or quantifies one or more biomarkers that binds to: an HA protein from influenza A H3, an HA protein from influenza A H1, an HA protein from influenza A H7, an HA protein from influenza B/Phuket; and an HA protein from influenza B/Brisbane. In embodiments, the multiplexed method simultaneously detects and/or quantifies one or more biomarkers that binds to: an HA protein from influenza A H3, an HA protein from influenza A H1, an HA protein from influenza A H7, an HA protein from influenza B/Phuket; an HA protein from influenza B/Brisbane; and/or an F protein from RSV. In embodiments, the multiplexed method simultaneously detects and/or quantifies one or more biomarkers that binds to: an HA protein from influenza A/Hong Kong H3, an HA protein from influenza A/Michigan H1, an HA protein from influenza A/Shanghai H7, an HA protein from influenza B/Phuket; an HA protein from influenza B/Brisbane; and an F protein from RSV. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, and/or an HA from influenza A H3. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, and an HA from influenza A/Hong Kong H3. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, an HA from influenza A H3, an HA protein from influenza A H1, an HA protein from influenza A H7, an HA protein from influenza B/Phuket; an HA protein from influenza B/Brisbane; and/or an F protein from RSV. In embodiments, the multiplexed method simultaneously detects and/or quantifies one or more biomarkers that binds to: an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, an HA from influenza A/Hong Kong H3, an HA protein from influenza A/Michigan H1, an HA protein from influenza A/Shanghai H7, an HA protein from influenza B/Phuket; an HA protein from influenza B/Brisbane; and an F protein from RSV. In embodiments, the one or more biomarkers is IgG, IgA, IgM, or combination thereof. In embodiments, the IgG, IgA, and/or IgM is from a human, mouse, rat, ferret, minx, bat, or combination thereof. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the multiplexed immunoassay method comprises: contacting the biological sample with a surface comprising a viral antigen in each binding domain on the surface, wherein the viral antigen in each binding domain is independently an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, or an F protein from RSV; forming a binding complex in each binding domain comprising the viral antigen and a biomarker that binds to the viral antigen; and measuring the concentration of the biomarker in each binding complex. In some embodiments, the S protein is a subunit, domain, or fragment thereof, e.g., S1, S2, S-NTD, S-ECD, or S-RBD. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1.

In embodiments, the multiplexed immunoassay method comprises: contacting the biological sample with a surface comprising a viral antigen in each binding domain on the surface, wherein the viral antigen in each binding domain is independently: an N protein from SARS-CoV-2, an S protein from SARS-CoV-2, an S-RBD from SARS-CoV-2, an S-NTD from SARS-CoV-2, an S protein from SARS-CoV, an S-protein from MERS-CoV, an S protein from HCoV-HKU1, an S protein from HcoV-OC43, an HA from influenza strain B/Brisbane, an HA from influenza strain B/Phuket, an HA from influenza strain H1/Michigan, an HA from influenza strain H3/Hong Kong, and an HA from influenza strain H7/Shanghai; forming a binding complex in each binding domain comprising the viral antigen and a biomarker that binds to the viral antigen; and measuring the concentration of the biomarker in each binding complex. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the immunoassay method is a classical serology assay. In embodiments, the immunoassay method is a bridging serology assay. Classical and bridging serology assays are provided herein. In embodiments, the concentration of the biomarker is measured by providing a detectable competitor of the biomarker, e.g., a natural interacting partner of the viral antigen, and measuring the decrease in competitor-viral antigen binding as the biomarker competes with the competitor for binding to the viral antigen. In embodiments, the competitor is ACE2. In embodiments, the competitor is NRP1. Competitive assays are further described herein.

In embodiments, the multiplexed immunoassay method comprises: contacting the biological sample with a surface comprising a viral antigen in each binding domain on the surface, wherein the viral antigen in each binding domain is independently: an N protein from SARS-CoV-2, an S-NTD from SARS-CoV-2, an S-RBD from SARS-CoV-2, an S protein from SARS-CoV-2, and an S protein from SARS-CoV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the immunoassay method is a classical serology assay. In embodiments, the immunoassay method is a bridging serology assay. Classical and bridging serology assays are provided herein. In embodiments, the concentration of the biomarker is measured by providing a detectable competitor of the biomarker, e.g., a natural interacting partner of the viral antigen, and measuring the decrease in competitor-viral antigen binding as the biomarker competes with the competitor for binding to the viral antigen. In embodiments, the competitor is ACE2. In embodiments, the competitor is NRP1. Competitive assays are further described herein. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 3 of FIG. 39B comprises an immobilized SARS-CoV-2 N protein, Spot 4 of FIG. 39B comprises an immobilized SARS-CoV S protein, Spot 8 of FIG. 39B comprises an immobilized SARS-CoV-2 S-NTD protein, Spot 10 of FIG. 39B comprises an immobilized SARS-CoV-2 S-RBD, and Spots 2, 5, 6, 7, and 9 of FIG. 39B each comprises an immobilized BSA.

In embodiments, the multiplexed immunoassay method comprises: contacting the biological sample with a surface comprising a viral antigen in each binding domain on the surface, wherein the viral antigen in each binding domain is independently: an N protein from SARS-CoV-2, an S-RBD from SARS-CoV-2, and an S protein from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 3 of FIG. 39B comprises an immobilized SARS-CoV-2 N protein, Spot 10 of FIG. 39B comprises an immobilized SARS-CoV-2 S-RBD, and Spots 2, 4, 5, 6, 7, 8, and 9 of FIG. 39B each comprises an immobilized BSA. In embodiments, the assay plate is a 384-well assay plate. An embodiment of a well in a 384-well assay plate, comprising four binding domains (“spots”), is shown in FIG. 39A. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized SARS-CoV-2 S-RBD, and Spot B2 of FIG. 39A comprises an immobilized BSA.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an N protein from SARS-CoV-2, an S-RBD from SARS-CoV-2 strain 501Y.V2, and an S protein from SARS-CoV-2 strain 501Y.V2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, w % herein each well comprises four distinct binding domains. An embodiment of a well in a 384-well assay plate, comprising four binding domains (“spots”), is shown in FIG. 39A. In embodiments, Spot A1 of FIG. 39A comprises an immobilized wild-type S protein from SARS-CoV-2, Spot A2 of FIG. 39A comprises an immobilized N protein from SARS-CoV-2, Spot B1 of FIG. 39A comprises an immobilized S-RBD from SARS-CoV-2 strain 501Y.V2, and Spot B2 of FIG. 39A comprises an immobilized S protein from SARS-CoV-2 strain 501Y.V2.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized Orf8 oligomer protein from SARS-CoV-2, Spot 3 of FIG. 39B comprises an immobilized N protein from immobilized SARS-CoV-2, Spot 4 of FIG. 39B comprises an immobilized Mem protein from SARS-CoV-2, Spot 6 of FIG. 39B comprises an immobilized Orf7a protein from SARS-CoV-2, Spot 7 of FIG. 39B comprises an immobilized Env protein from SARS-CoV-2, Spot 9 of FIG. 39B comprises an immobilized Orf8 monomer protein from SARS-CoV-2. Spot 10 of FIG. 39B comprises an immobilized wild-type S-RBD from SARS-CoV-2, Spot 5 of FIG. 39B comprises an immobilized BSA, and Spot 8 of FIG. 39B comprises an immobilized human serum albumin (HSA).

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 2, 4, 5, 6, and 10 of FIG. 39B each comprises an immobilized BSA.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2. Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spots 4, 5, and 6 of FIG. 39B each comprises an immobilized BSA.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 4, 5, 6, and 10 of FIG. 39B each comprises an immobilized BSA.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain 501Y.V2, Spot 3 of FIG. 39B comprises an immobilized N protein from SARS-CoV-2, Spot 4 of FIG. 39B comprises an immobilized S-RBD from SARS-CoV-2 strain P.1, Spot 6 of FIG. 39B comprises an immobilized S-RBD from SARS-CoV-2 strain B.1.1.7, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2. Spot 10 of FIG. 39B comprises an immobilized wild-type S-RBD from SARS-CoV-2, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA. IgM, or combination thereof. In embodiments, the IgG. IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2. Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429. Spot 10 of FIG. 39B comprises an immobilized wild-type S-RBD from SARS-CoV-2, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and an HA from influenza A/Hong Kong H3; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized influenza A/Hong Kong H3 HA protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized MERS-CoV S protein, Spot 10 of FIG. 39B comprises an immobilized SARS-CoV-2 S-RBD, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A H3, an HA from influenza A H1, an HA from influenza A H7, an HA from influenza B/Phuket, and an HA from influenza B/Brisbane; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG. IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises two assay plates. In embodiments, each assay plate is a 96-well plate.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and an S protein from HCoV-229E; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and an S protein from HCoV-229E; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG. IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized MERS-CoV S protein, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-RBD from SARS-CoV-2, an S protein from SARS-CoV-2, an S2 from SARS-CoV-2, an S protein from SARS-CoV, an S protein from HCoV-OC43, an S protein from HCoV-HKU1, an S protein from HCoV-NL63, and an S protein from HCoV-229E, forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S2, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized S protein from SARS-CoV-2, Spot 2 of FIG. 39B comprises an immobilized S1 from HCoV-NL63, Spot 3 of FIG. 39B comprises an immobilized N protein from SARS-CoV-2, Spot 4 of FIG. 39B comprises an immobilized S1 from SARS-CoV, Spot 6 of FIG. 39B comprises an immobilized S1 from SARS-CoV-2, Spot 7 of FIG. 39B comprises an immobilized S1 from HCoV-HKU1, Spot 8 of FIG. 39B comprises an immobilized S1 from HCoV-OC43, Spot 9 of FIG. 39B comprises an immobilized S1 from HCoV-229E, Spot 10 of FIG. 39B comprises an immobilized S-RBD from SARS-CoV-2, and Spot 5 of FIG. 39B comprises an immobilized BSA.

In embodiments, the multiplexed immunoassay method comprises: contacting the biological sample with a surface comprising a viral antigen in each binding domain on the surface, wherein the viral antigen in each binding domain is independently an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG. IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized N protein from HCoV-NL63, Spot 3 of FIG. 39B comprises an immobilized N protein from SARS-CoV-2, Spot 4 of FIG. 39B comprises an immobilized N protein from SARS-CoV, Spot 6 of FIG. 39B comprises an immobilized N protein from MERS-CoV, Spot 7 of FIG. 39B comprises an immobilized N protein from HCoV-HKU1, Spot 8 of FIG. 39B comprises an immobilized N protein from HCoV-OC43, Spot 9 of FIG. 39B comprises an immobilized N protein from HCoV-229E, Spot 10 of FIG. 39B comprises an immobilized S-RBD from SARS-CoV-2, and Spot 5 of FIG. 39B comprises an immobilized BSA

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG. IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises two assay plates. In embodiments, each assay plate is a 96-well plate.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises two assay plates. In embodiments, each assay plate is a 96-well plate.

In embodiments, the multiplexed immunoassay method comprises: contacting the biological sample with a surface comprising a viral antigen in each binding domain on the surface, wherein the viral antigen in each binding domain is independently an S protein from SARS-CoV-2, an S-RBD from SARS-CoV-2, an S-NTD from SARS-CoV-2, an N protein from SARS-CoV-2, an S protein from SARS-CoV, and an S protein from MERS-CoV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1.

In embodiments, the multiplexed immunoassay method comprises: contacting the biological sample with a surface comprising a viral antigen in each binding domain on the surface, wherein the viral antigen in each binding domain is independently an HA protein from FluB/Brisbane/60/2008, an HA protein from FluB/Phuket/3073/2013, an HA protein from FluA/Michigan/45/2015 (H1N1), an HA protein from FluA/HongKong/4801/2014 (H3N2), an HA protein from FluA/Shanghai/2/2013 (H7N9), an S protein from HCoV-HKU1, and an S protein from HCoV-OC43; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1.

In embodiments, the multiplexed immunoassay method comprises: contacting the biological sample with a surface comprising a viral antigen in each binding domain on the surface, wherein the viral antigen in each binding domain is independently an S protein from SARS-CoV-2, an S-RBD from SARS-CoV-2, an S-NTD from SARS-CoV-2, an N protein from SARS-CoV-2, an S protein from SARS-CoV, an S protein from MERS-CoV, an S protein from HCoV-HKU1, an S protein from HCoV-OC43, and an HA protein from FluA/HongKong/4801/2014 (H3N2); forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1.

In embodiments, the multiplexed immunoassay method comprises: contacting the biological sample with a surface comprising a viral antigen in each binding domain on the surface, wherein the viral antigen in each binding domain is independently an HA protein from influenza A H3, an HA protein from influenza A H1, an HA protein from influenza A H7, an HA protein from influenza B/Phuket; and an HA protein from influenza B/Brisbane; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments. Spot 1 of FIG. 39B comprises an immobilized influenza B/Brisbane HA protein, Spot 2 of FIG. 39B comprises an immobilized influenza A H7 (e.g., H7 Shanghai strain) HA protein, Spot 4 of FIG. 39B comprises an immobilized influenza A H1 (e.g., H1 Michigan strain) HA protein, Spot 8 of FIG. 39B comprises an immobilized influenza A H3 (e.g., H3/Hong Kong strain) protein, Spot 10 of FIG. 39B comprises an immobilized influenza B/Phuket HA protein, and Spots 3, 5, 6, 7, and 9 each comprises an immobilized BSA.

In embodiments, the multiplexed immunoassay method comprises: contacting the biological sample with a surface comprising a viral antigen in each binding domain on the surface, wherein the viral antigen in each binding domain is independently an HA protein from influenza A/Hong Kong H3, an HA protein from influenza A/Michigan H1, an HA protein from influenza A/Shanghai H7, an HA protein from influenza B/Phuket; an HA protein from influenza B/Brisbane, and an F protein (e.g., pre-fusion F protein) from RSV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized influenza B/Brisbane HA protein, Spot 2 of FIG. 39B comprises an immobilized influenza A/Shanghai H7 HA protein, Spot 4 of FIG. 39B comprises an immobilized influenza A/Michigan H1 HA protein, Spot 7 of FIG. 39B comprises an immobilized RSV pre-fusion F protein, Spot 8 of FIG. 39B comprises an immobilized influenza A/Hong Kong H3 protein, Spot 10 of FIG. 39B comprises an immobilized influenza B/Phuket HA protein, and Spots 3, 5, 6, and 9 each comprises an immobilized BSA.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, and/or an HA from influenza A H3; forming a binding complex in each binding domain comprising the viral antigen and a biomarker that binds to the viral antigen; and measuring the concentration of the biomarker in each binding complex. In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, and an HA from influenza A/Hong Kong H3; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG. IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises two assay plates. In embodiments, each assay plate is a 96-well plate.

In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, an HA from influenza A H3, an HA protein from influenza A H1, an HA protein from influenza A H7, an HA protein from influenza B/Phuket; an HA protein from influenza B/Brisbane; and/or an F protein from RSV; forming a binding complex in each binding domain comprising the viral antigen and a biomarker that binds to the viral antigen; and measuring the concentration of the biomarker in each binding complex. In embodiments, the multiplexed immunoassay method comprises: contacting the biological sample with a surface comprising a viral antigen in each binding domain on the surface, wherein the viral antigen in each binding domain is independently an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from NL63, an S protein from HCoV-229E, an HA from influenza A/Hong Kong H3, an HA protein from influenza A/Michigan H1, an HA protein from influenza A/Shanghai H7, an HA protein from influenza B/Phuket; an HA protein from influenza B/Brisbane; and an F protein from RSV; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1.

In embodiments, the invention provides an immunoassay method comprising: quantifying the amounts of one or more biomarkers capable of binding to a coronavirus viral antigen in a biological sample, wherein the viral antigen comprises a spike protein (S), spike protein subunit 1 (S1), spike protein subunit 2 (S2), spike protein N-terminal domain (S-NTD), spike protein ectodomain (S-ECD), spike protein receptor binding domain (S-RBD), membrane protein (M), envelope protein (E), nucleocapsid protein (N), or any combination thereof, wherein the quantifying comprises measuring the concentrations of each of the one or more biomarkers in an immunoassay. In embodiments, the coronavirus is SARS-CoV, MERS-CoV, SARS-CoV-2, HcoV-OC43, HcoV-229E, HcoV-NL63, HcoV-HKU1, or a combination thereof. In embodiments, the one or more biomarkers is capable of binding to the S protein from SARS-CoV-2, SARS-CoV. MERS-CoV. HcoV-OC43. HcoV-229E, HcoV-NL63, HcoV-HKU1, or a combination thereof. In embodiments, the one or more biomarkers binds to SARS-CoV-2 S-D614. In embodiments, the one or more biomarkers binds to SARS-CoV-2 S-D614G. In embodiments, the one or more biomarkers binds to a SARS-CoV-2 S protein or subunit or fragment thereof that comprises a mutation as shown in Tables 1A and 1B. In embodiments, the one or more biomarkers binds to a SARS-CoV-2 N protein that comprises a mutation as shown in Table 1A.

Coronaviruses, such as SARS-CoV, MERS-CoV, SARS-CoV-2, HcoV-OC43, HcoV-229E, HcoV-NL63, and HcoV-HKU1, and their structural and nonstructural proteins are described herein. As discussed herein, a method that is capable of detecting a coronavirus, e.g., using a conserved coronavirus component, can be used to detect novel strains of coronavirus. Moreover, such a method can also aid in understanding a patient's immune response to different coronaviruses, e.g., a generally mild response to HcoV-OC43, HcoV-229E, HcoV-NL63, and HcoV-HKU1, compared to a generally severe or even lethal response to SARS-CoV and MERS-CoV, and a range of mild to severe responses to SARS-CoV-2.

In embodiments, the method is a multiplexed method capable of simultaneously quantifying the one or more biomarkers that bind to a coronavirus viral antigen. In embodiments, the multiplexed immunoassay method comprises: contacting the biological sample with a surface comprising a viral antigen in each binding domain on the surface, wherein the viral antigen in each binding domain is independently the S protein from SARS-CoV-2 (e.g., S-D614 and/or S-D614G), the S protein from SARS-CoV, the S protein from MERS-CoV, the S protein from HCoV-OC43, the S protein from HCoV-229E, the S protein from HCoV-NL63, or the S protein from HCoV-HKU1; forming a binding complex in each binding domain comprising the viral antigen and a biomarker that binds to the viral antigen; and measuring the concentration of the biomarker in each binding complex. In embodiments, the S protein is a subunit, domain, or fragment thereof, e.g., S1, S2, S-NTD, S-ECD, or S-RBD.

In embodiments, the immunoassay method comprises detecting one or more viral antigens that are specific to SARS-CoV-2. As discussed herein, SARS-CoV-2 causes the respiratory illness COVID-19, which can cause mild to severe symptoms in patients. Sensitive and specific detection of SARS-CoV-2 is important for providing an accurate diagnosis, identifying asymptomatic infected individuals, and tracking spread of the disease. A method that detects biomarkers produced by an individual in response to a SARS-CoV-2 infection (e.g., antibodies) is also useful for identifying those who may be immune to the virus and therefore may be at lower risk when interacting with the general public or infected patients, and also may be potential candidates for plasma transfusions. In embodiments, the one or more biomarkers is capable of binding to a SARS-CoV-2 S-D614 protein, S-D614G, S1 subunit, S2 subunit, S-NTD, S-RBD, M protein, E protein, N protein, or a combination thereof. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the method is a multiplexed method capable of simultaneously quantifying the one or more biomarkers that bind to a SARS-CoV-2 antigen. In embodiments, the multiplexed immunoassay method comprises: contacting the biological sample with a surface comprising a viral antigen in each binding domain on the surface, wherein the viral antigen in each binding domain is independently the SARS-CoV-2 S-D614, S-D614G, the SARS-CoV-2 S1 subunit, the SARS-CoV-2 S2 subunit, the SARS-CoV-2 S-RBD, the SARS-CoV-2 S-ECD, the SARS-CoV-2 S-NTD, the SARS-CoV-2 M protein, the SARS-CoV-2 E protein, or the SARS-CoV-2 N protein. In embodiments, the method is capable of simultaneously detecting a biomarker that binds to at least one of the SARS-CoV-2 S-D614, S-D614G, the SARS-CoV-2 S1 subunit, the SARS-CoV-2 S2 subunit, the SARS-CoV-2 S-RBD, the SARS-CoV-2 S-ECD, the SARS-CoV-2 S-NTD, the SARS-CoV-2 M protein, the SARS-CoV-2 E protein, and the SARS-CoV-2 N protein. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the immunoassay comprises: (a) contacting the biological sample with the viral antigen that specifically binds to a first biomarker of the one or more biomarkers; (b) forming a binding complex comprising the viral antigen and the first biomarker; and (c) measuring the concentration of the first biomarker in the binding complex.

In embodiments, the method further comprises repeating one or more of the method steps described herein to quantify the amounts of one or more biomarkers in the sample. In embodiments, the method further comprises repeating steps (a)-(c), wherein each biomarker specifically binds to a different viral antigen, thereby quantifying one or more biomarkers. In embodiments, each of steps (a)-(c) is performed for each biomarker in parallel.

In embodiments, the method is a multiplexed method. In embodiments, the multiplexed method is capable of simultaneously quantifying at least two biomarkers in the biological sample, wherein each of the at least two biomarkers is independently capable of binding to a viral antigen, e.g., any of HA, F, S, S1, S2, S-NTD, S-ECD, S-RBD, M, E, or N as described herein. In embodiments, the multiplexed method is capable of simultaneously quantifying two, three, four, five, or more than five biomarkers in the biological sample, wherein each biomarker is independently capable of binding to a viral antigen, e.g., any of HA, F, S, S1, S2, S-NTD, S-ECD, S-RBD, M, E, or N as described herein. In embodiments, the multiplexed method comprising quantifying a combination of the biomarkers provided herein has improved sensitivity and/or dynamic range, compared to a method in which only a single biomarker is quantified. For example, a multiplexed method can provide earlier and more sensitive detection compared to a method that detects a single biomarker, since responses to each viral antigen may vary between individuals. Moreover, the ability to simultaneously measure antibody responses against multiple similar viruses, e.g., a newly-emerged coronavirus such as SARS-CoV-2 and similar coronaviruses viruses such as HCoV-OC43, HCoV-HKU1, and HCoV-NL63, which have been circulating in the general population, improves understanding of how an individual's prior exposure to similar circulating viruses affects the individual's response to the newly-emerged virus of interest.

In embodiments, the method is used to diagnose whether a subject is infected with a virus, e.g., SARS-CoV-2. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is a coronavirus. In embodiments, the virus is SARS-CoV-2.

In embodiments, the method is used to identify individuals with previous virus exposure for epidemiological studies (e.g., to understand true disease prevalence and evaluate the efficacy of infection control measures). In embodiments, the method is used to identify individuals at lower risk of future infection. Moreover, the method can be an important tool in the research, development, and validation of a vaccine for the virus. In embodiments, the method is used to assess differences in immune responses (e.g., antibody response) between individuals whose immunity is achieved by natural infection or vaccination. For example, a multiplexed method differentiates an individual's response to vaccination with different constructs of a viral antigen (e.g., different fragments of the S protein), compared with the individual's response to natural infection by the virus. Such a method can advantageously distinguish between individuals with biomarkers produced in response an active infection and are potentially contagious and individuals with biomarkers produced in response to the vaccine. In embodiments, the virus is a coronavirus. In embodiments, the virus is SARS-CoV-2.

In embodiments, the biomarker capable of binding to a viral antigen is an immune biomarker. In embodiments, the biomarker is an antibody or antigen-binding fragment thereof. In embodiments, the biomarker is an immunoglobulin A (IgA), immunoglobulin G (IgG; including IgG subclasses IgG1, IgG2, IgG3, and IgG4), immunoglobulin M (IgM), immunoglobulin E (IgE), or immunoglobulin D (IgD), or antigen-binding fragments thereof capable of binding to S. S1, S2, S-NTD, S-ECD, S-RBD, M, E, and/or N. In embodiments, the IgG, IgA, IgM, IgD, and/or IgE is from a human, mouse, rat, ferret, minx, bat, or combination thereof. In embodiments, the biomarker is an IgA or antigen-binding fragment thereof capable of binding to S, S1, S2, S-NTD, S-ECD, S-RBD, M, E. and/or N. In embodiments, the biomarker is an IgG or antigen-binding fragment thereof capable of binding to S. S1, S2, S-NTD, S-ECD, S-RBD, M, E, and/or N. In embodiments, the biomarker is an IgG1 or antigen-binding fragment thereof capable of binding to S, S1, S2, S-NTD, S-ECD, S-RBD, M. E, and/or N. In embodiments, the biomarker is an IgG2 or antigen-binding fragment thereof capable of binding to S, S1, S2, S-NTD, S-ECD, S-RBD, M, E, and/or N. In embodiments, the biomarker is an IgG3 or antigen-binding fragment thereof capable of binding to S, S1. S2, S-NTD, S-ECD, S-RBD, M, E, and/or N. In embodiments, the biomarker is an IgG4 or antigen-binding fragment thereof capable of binding to S, S1, S2, S-NTD, S-ECD, S-RBD, M, E. and/or N. In embodiments, the biomarker is an IgM or antigen-binding fragment thereof capable of binding to S, S1, S2, S-NTD, S-ECD. S-RBD, M, E. and/or N. In embodiments, the biomarker is an IgE or antigen-binding fragment thereof capable of binding to S, 51, S2, S-NTD, S-ECD, S-RBD, M, E, and/or N. In embodiments, the biomarker is an IgD or antigen-binding fragment thereof capable of binding to S, S1, S2, S-NTD, S-ECD, S-RBD, M, E. and/or N. In embodiments, the viral antigen is a coronavirus antigen. In embodiments, the coronavirus is SARS-CoV-2. In embodiments, the biomarker binds to SARS-CoV-2 S-D614. In embodiments, the biomarker binds to SARS-CoV-2 S-D614G. In embodiments, the biomarker binds to a SARS-CoV-2 S protein or subunit or fragment thereof that comprises a mutation as shown in Tables 1A and 1B. In embodiments, the biomarker binds to a SARS-CoV-2 N protein that comprises a mutation as shown in Table 1A.

In embodiments, the biomarker to be detected is an antibody biomarker, and the binding reagent is a viral antigen that is bound by the antibody biomarker. In embodiments, the binding reagent is a viral protein described herein, e.g., HA, F, S, S1, S2, S-NTD, S-ECD, S-RBD, M, E, N.

In embodiments, the binding reagent is a peptide antigen. Peptide antigens are short peptides of a native, full-length protein that include the antibody binding epitope. Peptide antigens can be easier to produce and provide greater flexibility in performing an immunoassay to detect an antibody biomarker. Peptide antigens can also have higher specificity to the antibody biomarker compared with a full-length viral protein or domain described herein. In embodiments, an immunoassay utilizing a peptide antigen as the binding reagent has reduced cross-reactivity with antibody biomarkers for a different virus that are present in the biological sample. For example, an immunoassay utilizing a SARS-CoV-2 peptide antigen can have reduced cross-reactivity for antibodies that may be present in a subject for a circulating coronavirus.

In embodiments, the peptide antigen is a fragment of a viral protein, e.g., a coronavirus protein. In embodiments, the peptide antigen comprises about 10 to about 100 amino acids. In embodiments, the peptide antigen comprises about 20 to about 80 amino acids. In embodiments, the peptide antigen comprises about 30 to about 60 amino acids. In embodiments, the peptide antigen comprises about 40 to about 50 amino acids. In embodiments, the peptide antigen is a fragment of S, S1, S2, S-NTD, S-ECD, S-RBD, M, E, or N. In embodiments, the peptide antigen comprises an immunodominant region (IDR) of a viral protein. In embodiments, the peptide antigen comprises amino acids 1-49 of the N protein IDR. In embodiments, the peptide antigen comprises amino acids 340-390 of the N protein IDR. In embodiments, the peptide antigen comprises amino acids 192-220 of the of the N protein IDR. In embodiments, the peptide antigen comprises amino acids 182-216 of the M protein IDR.

IgA, IgG (and subclasses thereof), IgM, IgE, and IgD are different isotypes of antibodies that have different immunological properties and functional locations. For example, IgA is typically found in the mucosal areas, such as the respiratory and gastrointestinal tracts, saliva, and tears and can prevent colonization by pathogens. IgG, the most abundant antibody isotype, has four subclasses as described herein and is found in all bodily fluids and provides the majority of antibody-based immunity against pathogens. IgM is mainly found in the blood and lymph fluid and is typically the first antibody made by the body to fight a new infection. IgE is mainly associated with allergic reactions (e.g., as part of aberrant immune response) and is found in the lungs, skin, and mucous membranes. IgD mainly functions as an antigen receptor on B cells and may activate basophils and mast cells to produce antimicrobial factors. Based on the timing and/or type of infection, different amounts of each isotype are produced.

In embodiments, the method is a multiplexed immunoassay method capable of quantifying the amount of each isotype of antibodies, e.g., IgG, IgA, IgE, and IgM, present in the biological sample. In embodiments, the amounts of the different isotypes of antibodies measured in a biological sample. e.g., the amounts of each of IgG, IgA, IgE, and IgM, can be used to determine whether a subject has been previously exposed to a virus. In embodiments, the amounts of the different isotypes of antibodies measured in a biological sample, e.g., the amounts of each of IgG, IgA, IgE, and IgM, can be used to estimate the time of virus exposure and/or infection. In embodiments, the amounts of the different isotypes of antibodies measured in a biological sample. e.g., the amounts of each of IgG, IgA, IgE, and IgM, can be used to determine whether a subject has immunity to a virus, e.g., a coronavirus such as SARS-CoV-2.

In embodiments, the method comprises: (a) contacting the biological sample with: at least a first, second, third, and fourth viral antigens, wherein each viral antigen specifically binds to IgG, IgA, IgE, and IgM, respectively; (b) forming at least a first, second, third, and fourth binding complex comprising the viral antigens and IgG. IgA, IgE, or IgM; and (c) measuring the concentration of IgG, IgA, IgE, or IgM in each of the binding complexes. In embodiments, each viral antigen is independently S, S1, S2, S-NTD, S-ECD, S-RBD, M, E, N, or a peptide antigen described herein. In embodiments, the IgG, IgA, IgE, and/or IgM is from a human, mouse, rat, ferret, minx, bat, or combination thereof.

IgG is further divided into four subclasses, IgG1, IgG2, IgG3, and IgG4, based on properties such as ability to activate complement, bind to macrophages, and/or pass through the placenta. Each subclass also has a distinct biological function. For example, the response to protein antigens is primarily mediated by IgG1 and IgG3, while IgG2 primarily mediates the response to polysaccharide antigens. IgG4 plays a role in protection against certain hypersensitivity reactions and pathogenesis of some autoimmune diseases. IgG subclass screening is performed to monitor a subject's infection response and/or determine whether a subject has antibody deficiency, and/or assess a subject's risk of an adverse response to infection. In embodiments, the method comprises determining the amount of IgG1, IgG2, IgG3, and IgG4 in the biological sample. In embodiments, the IgG is from a human, mouse, rat, ferret, minx, bat, or combination thereof.

In embodiments, the method comprises: (a) contacting the biological sample with: at least a first, second, third, and fourth viral antigens, wherein each viral antigen specifically binds to IgG1, IgG2, IgG3, and IgG4 respectively; (b) forming at least a first, second, third, and fourth binding complex comprising the viral antigens and IgG1, IgG2, IgG3, or IgG4; and (c) measuring the concentration of IgG1, IgG2, IgG3, or IgG4 in each of the binding complexes. In embodiments, each viral antigen is independently S, S1, S2, S-NTD, S-ECD, S-RBD, M, E, N, or a peptide antigen described herein. In embodiments, the IgG is from a human, mouse, rat, ferret, minx, bat, or combination thereof.

In embodiments, the method comprises: (a) contacting the biological sample with: a plurality of viral antigens, wherein each viral antigen specifically binds to an immunoglobulin selected from IgG1, IgG2, IgG3, IgG4, IgA, IgE, and IgM; (b) forming a plurality of binding complexes comprising the viral antigens and immunoglobulins; and (c) measuring the concentration of the immunoglobulin in each of the binding complexes. In embodiments, each viral antigen is independently S, S1, S2, S-NTD, S-ECD, S-RBD, M, E, N, or a peptide antigen described herein. In embodiments, the IgG, IgA, IgE, and/or IgM is from a human, mouse, rat, ferret, minx, bat, or combination thereof.

Inflammatory/Tissue Damage Response Biomarkers

In embodiments, the invention provides a method for detecting a biomarker in a subject to detect a viral infection, e.g., by a respiratory virus, including coronaviruses such as SARS-CoV-2. In embodiments, the invention provides a method for detecting a biomarker in a subject to assess the severity and/or prognosis of a viral infection, e.g., by a respiratory virus, including coronaviruses such as SARS-CoV-2. In embodiments, the biomarker is produced in response to the viral infection. In embodiments, the biomarker is a stress response protein. In embodiments, the biomarker is an inflammatory response biomarker. In embodiments, the biomarker is a tissue damage response biomarker. In embodiments, the biomarker is a T cell activation biomarker. In embodiments, the biomarker is an extracellular vesicle.

In embodiments, the invention provides an immunoassay method comprising: quantifying the amounts of one or more biomarkers in a biological sample, wherein the one or more biomarkers comprises C-reactive protein (CRP), IFNα2, IFN-γ, IL-6, IL-10, MCP-1, IP-10, troponin (e.g., skeletal troponin-I (sTnI)), IL-1β, IL-2, IL-4, IL-7, granulocyte colony-stimulating factor (G-CSF), MIP-1α, TNF-α, ferritin, CD147, neurofilament light (NfL), kidney injury molecule-1 (KIM-1), IL-8, MIP-10, MCP-4, thymus and activation regulated chemokine (TARC, also known as CCL17), vascular endothelial growth factor receptor-1 (VEGFR-1, also known as Flt-1), phosphatidylinositol-glycan biosynthesis class F protein (PIGF), vascular endothelial growth factor A (VEGF-A), vascular endothelial growth factor C (VEGF-C), intercellular adhesion molecule 1 (ICAM-1), serum amyloid A (SAA), vascular cell adhesion protein 1 (VCAM-1), angiopoietin-2 (Ang-2), Lectin Binding Protein (LBP), an inflammatory damage biomarker, a tissue damage biomarker, a T cell activation biomarker, an extracellular vesicle, or any combination thereof, wherein the quantifying comprises measuring the concentrations of each of the one or more biomarkers in an immunoassay.

In embodiments, the immunoassay method comprises: (a) contacting the biological sample with a binding reagent that specifically binds to a first biomarker of the one or more biomarkers; (b) forming a binding complex comprising the binding reagent and the first biomarker; and (c) measuring the concentration of the first biomarker in the binding complex. In embodiments, the method further comprises repeating one or more of the method steps described herein to quantify the amounts of one or more biomarkers in the sample. In embodiments, the method further comprises repeating steps (a)-(c), wherein each biomarker specifically binds to a different binding reagent, thereby quantifying one or more biomarkers. In embodiments, each of steps (a)-(c) is performed for each biomarker in parallel. In embodiments, the one or more biomarkers comprises C-reactive protein (CRP), IFNα2, IFN-γ, IL-6, IL-10, MCP-1, IP-10, troponin (e.g., sTnI). IL-10, IL-2, IL-4, IL-7, G-CSF, MIP-1α, TNF-α, ferritin. CD147, NfL, KIM-1, IL-8, MIP-1R, MCP-4, TARC, Flt-1, PIGF, VEGF-A, VEGF-C, ICAM-1, SAA, VCAM-1, Ang-2, LBP, an inflammatory response biomarker, a tissue damage biomarker, T cell activation biomarker, an extracellular vesicle, or any combination thereof. In embodiments, the biomarker is G-CSF. In embodiments, the biomarker is GM-CSF. In embodiments, the biomarker is IFN-α2a. In embodiments, the biomarker is IL-4. In embodiments, the biomarker is IL-6. In embodiments, the biomarker is IL-10. In embodiments, the biomarker is TNF-α. In embodiments, the biomarker is ferritin. In embodiments, the biological sample is obtained from a human subject.

In embodiments, the immunoassay method is a multiplexed method capable of simultaneously quantifying at least two biomarkers in the biological sample. In embodiments, the multiplexed method is capable of simultaneously quantifying the amount of at least 2, at least 3, at least 4, at least 5, at least 6, at least 7, at least 8, at least 9, or at least 10 biomarkers described herein. In embodiments, the multiplexed method comprising quantifying a combination of the biomarkers provided herein has improved assay sensitivity and/or dynamic range, compared to a method in which only a single biomarker is quantified. In embodiments, the multiplexed method is capable of simultaneously quantifying one or more of CRP, IFNα2, IFN-γ, IL-6, IL-10, MCP-1, IP-10, troponin (e.g., sTnI), IL-10, IL-2. IL-4, IL-7, G-CSF, MIP-1α, TNF-α, ferritin, CD147, NIL, KIM-1, IL-8, MIP-10, MCP-4, TARC, Flt-1, PIGF, VEGF-A, VEGF-C, ICAM-1, SAA, VCAM-1, Ang-2, LBP, an inflammatory response biomarker, a tissue damage biomarker, T cell activation biomarker, and an extracellular vesicle.

In embodiments, the immunoassay method simultaneously detects and/or quantifies IL-1p, IL-6, IL-8, and TNF-α in a biological sample. In embodiments, the immunoassay method simultaneously detects and/or quantifies IFN-γ, IL-1β, IL-4, IL-6, IL-8, IL-10, and TNF-α in a biological sample. In embodiments, the immunoassay method simultaneously detects and/or quantifies IFN-γ, IL-IP. IL-4, IL-5, IL-6, IL-8, IL-10, IL-12p70, and TNF-α in a biological sample. In embodiments, the immunoassay method simultaneously detects and/or quantifies IFN-γ, IL-1β, IL-2, IL-4, IL-5, IL-6, IL-8, IL-10, IL-12p70, IL-13, and TNF-α in a biological sample. In embodiments, the biological sample is obtained from a human subject.

In embodiments, the immunoassay method simultaneously detects and/or quantifies CRP, ICAM-1, SAA, and VCAM-1 in a biological sample. In embodiments, the immunoassay method simultaneously detects and/or quantifies CRP. LBP, ICAM-1, SAA, and VCAM-1 in a biological sample. In embodiments, the immunoassay method simultaneously detects and/or quantifies CRP, LBP, Eotaxin, Eotaxin-3, FGF (basic). VEGFR-1/Flt-1, GM-CSF, ICAM-1, IFN-γ, IL-1α, IL-1β, IL-2, IL-3, IL-4, IL-5, IL-6, IL-7, IL-8, IL-8 (HA), IL-9, IL-10, IL-12/IL-23p40, IL-12p70, IL-13, IL-15, IL-16, IL-17A, IL-17A/F, IL-17B, IL-17C, IL-17D, IL-1RA, IL-21, IL-22, IL-23, IL-27, IL-31, IP-10, MCP-1, MCP-4, MDC, MIP-1α, MIP-10, MIP-3a, PlGF, SAA, TARC, Tie-2, TNF-α, TNF-β, TSLP, VCAM-1, VEGF-A, VEGF-C, and VEGF-D in a biological sample. In embodiments, the biological sample is obtained from a human subject.

In embodiments, the immunoassay method simultaneously detects and/or quantifies IL-1, IL-6, and IL-8 in a biological sample. In embodiments, the immunoassay method simultaneously detects and/or quantifies IFN-γ, IL-10, IL-6, IL-8, and IL-10 in a biological sample. In embodiments, the immunoassay method simultaneously detects and/or quantifies IFN-γ, IL-1β, IL-5, IL-6, IL-8, and IL-10 in a biological sample. In embodiments, the immunoassay method simultaneously detects and/or quantifies IFN-γ, IL-1β, IL-2, IL-6, IL-8, and IL-10 in a biological sample. In embodiments, the biological sample is obtained from a non-human primate (NHP) subject.

In embodiments, the immunoassay method simultaneously detects and/or quantifies G-CSF, GM-CSF, IFN-α2a, IFN-D. IFN-γ, IL-1RA, IL-1D, IL-4, IL-5, IL-6, IL-7, IL-8, IL-9, IL-10, IL-12p70, IP-10, MCP1, MIP-1α, TNF-α, and VEGF-A in a biological sample. In embodiments, the immunoassay method simultaneously detects and/or quantifies GM-CSF, IL-1α, IL-5, IL-7, IL-12/IL-23p40, IL-15, IL-16, IL-17A, TNF-0, and VEGF-A in a biological sample. In embodiments, the biological sample is obtained from a human subject.

In embodiments, the immunoassay method simultaneously detects and/or quantifies G-CSF, GM-CSF, IFN-α2a, IFN-γ, IL-1RA, IL-10, IL-4, IL-6, IL-7, IL-8, IL-9, IL-10, IL-12p70, IP-10, MCP1, MIP-1α, TNF-α, and VEGF-A in a biological sample. In embodiments, the immunoassay method simultaneously detects and/or quantifies GM-CSF, IL-1α, IL-5, IL-7, IL-12/IL-23p40, IL-15, IL-16, IL-17A, TNF-β, VEGF-A in a biological sample. In embodiments, the biological sample is obtained from a non-human primate (NHP) subject.

In embodiments, the immunoassay method simultaneously detects and/or quantifies IFN-γ, IL-1β, IL-2, IL-4, IL-6, IL-10, IL-12p70, IL-17A, and TNF-α in a biological sample. In embodiments, the immunoassay method simultaneously detects and/or quantifies GFAP. Tau, and NF-L in a biological sample. In embodiments, immunoassay detecting and/or quantifying GFAP, Tau, and NF-L is detected and/or quantified is an ultrasensitive assay. In embodiments, the biological sample is obtained from a human subject. In embodiments, the method assesses the neurological effects of an infection by a virus described herein, e.g., SARS-CoV-2. In embodiments, the method assesses the neurological effects in a chronic illness caused by a virus described herein, e.g., post-acute COVID-19 syndrome caused by SARS-CoV-2 infection.

In embodiments, the immunoassay method simultaneously detects and/or quantifies CD78, CD28, CD40L, CTLA-4, GITR, LAG3, OX40. PD1, TIGIT, Tie-2, gp130, and TIM-3 in a biological sample. In embodiments, the immunoassay method simultaneously detects and/or quantifies CD78, CD28, CD40L, CTLA-4, GITR, LAG3, OX40, PD1, and TIGIT in a biological sample. In embodiments, the immunoassay method simultaneously detects and/or quantifies Tie-2, gp130, and TIM-3 in a biological sample. In embodiments, the biological sample is obtained from a human subject. In embodiments, the method assesses the immune checkpoint response of a subject infected with a virus described herein, e.g., SARS-CoV-2.

In embodiments, the method is used to assess the severity of a viral infection in a subject. In embodiments, the method is used to determine the prognosis of a viral infection in a subject. In a clinical setting, the present method can provide a useful triage screening tool to identify the highest risk patients and devise appropriate treatments.

In embodiments, the biomarker is an inflammatory response biomarker. An inflammatory response biomarker is a biomarker that is up- or down-regulated during systemic or localized inflammatory response, e.g., caused by a viral infection. In embodiments, the inflammatory response biomarker is IL-1, IL-2, IL-3, IL-4, IL-5, IL-6, IL-7, IL-10. IL-12, IL-23, TNF-α, INF-γ, C-reactive protein (CRP), monocyte chemoattractant protein-1 (MCP1, also known as CCL2), interferon-gamma induced protein 10 (IP-10, also known as CXCL10), serum amyloid A (SAA), CXCL1 (also known as KC/GRO), or any combination thereof. In embodiments, the inflammatory response biomarker is CRP, INF-γ, IL-6, IL-10, MCP-1, IP-10, IL-1β, IL-2, IL-4, IL-7, G-CSF, MIP-1α, TNF-α, ferritin, NfL, KIM-1, or any combination thereof.

In embodiments, the biomarker is a tissue damage biomarker. A tissue damage biomarker is a biomarker released from a tissue as a result of local tissue damage, e.g., caused by a viral infection. In embodiments, the tissue damage biomarker is troponin, salivary amylase, citrullinated proteins, creatine kinase BB (CKBB), creatine kinase MB (CKMB), creatine kinase MM (CKMM), S100B, surfactant protein D (SP-D), fatty acid binding protein 2 (FABP2), bacterial/permeability-increasing protein (BPI), glial fibrillary acidic protein (GFAP), thrombospondin (TSP), neuron-specific enolase (NSE), cancer antigen 15-3 (CA15-3), troponin (e.g., sTnI), or a combination thereof.

In embodiments, the biomarker is a T cell activation biomarker. A T cell activation biomarker is a biomarker that is up- or down-regulated during T cell activation, e.g., as part of an immune response. Non-limiting examples of types of T cells include helper CD4+ T cell, cytotoxic CD8+ T cell, memory T cell, regulatory CD4+ T cell, natural killer T cell, mucosal associated invariant T cell, and gamma delta T cell. In embodiments, the T cell is a helper CD4+ T cell. In embodiments, the T cell is a cytotoxic CD8+ T cell. In embodiments, the T cell activation biomarker is CD3, CD4, CD8, CD24, CD25, CD27, CD28, CD30, CD38, CD44, CD45RA, CD45RO, CD47, CD62L, CD69, CD94, CD107a, CD137, CD154, CD161, CD183, CD184, CD185 (CXCR5), CD193, CD194 (CCR4), CD195, CD196 (CCR6), CC197 (CCR7), CCR10, CXCR3, KLRG1, HLA-DR, AhR, TCRα/β, T-bet, STAT1, STAT3, STAT4. STAT5, GATA3, RORyt, IRF4, PU.1, BNC2, FOXO4, Bcl6, FoxP3, Smad2, IL-2, IFN-γ, TNF-α, IL-2, IL-4, IL-5, IL-6, IL-9, IL-10, IL-13, IL-12, IL-17A, IL-17F, IL-21, IL-22, IL-26, IL-35, TGF-β1, or a combination thereof.

In embodiments, the biomarker is a brain injury biomarker. A brain injury biomarker is a biomarker that is up- or down-regulated at any time from immediately after a brain injury to days, weeks, months, years, or decades after the brain injury. In embodiments, the brain injury is caused by a virus described herein, e.g., SARS-CoV-2. For example, SARS-CoV-2 has been shown to cause encephalitis, stroke, seizure, and other cognitive impairments such as memory loss, attention deficiency, anosmia, ageusia, and the like. In embodiments, the brain injury comprises encephalitis, stroke, seizure, hypoxic brain injury, or a combination thereof. In embodiments, the brain injury biomarker is present in plasma, saliva, or cerebrospinal fluid. In embodiments, the brain injury biomarker is detectable during early stages of brain injury, thereby allowing early intervention and treatment. In embodiments, the brain injury biomarker is neuron-specific enolase (NSE), brain-derived neurotrophic factor (BDNF), S100 calcium-binding protein B (S100B), monocyte chemoattractant protein 1 (MCP1), intercellular adhesion molecule-5 (ICAM-5), visinin-like protein 1 (VILIP-1), matrix metalloproteinase 9 (MMP-9), neuronal pentraxin 1 (NPTX1), neurogranin (NRGN) peroxiredoxin-6 (PRDX6), ubiquitin carboxyl-terminal esterase-L1 (UCHL 1), creatine kinase B type (CKBB), von Willebrand factor (vWF), glial fibrillary acidic protein (GFAP), Tau (including phosphorylated and non-phosphorylated Tau), neurofilament light chain (NF-L), or a combination thereof. In embodiments, the Tau is phosphorylated (p-Tau). In embodiments, the p-Tau is phosphorylated at amino acid position T175, T181, T212, S214, cis T231, trans T231, S293, S396, S610, or a combination thereof (amino acid positions corresponding to human Tau protein, NCBI accession no. NP_005901.2). Methods of detecting total Tau and/or p-Tau are described in, e.g., U.S. Provisional Application No. 63/111,333, filed Nov. 9, 2020.

In embodiments, the biomarker is an immune checkpoint biomarker. An immune checkpoint biomarker regulates the immune system and are important for self-tolerance, which prevents the immune system from indiscriminately attacking cells. Non-limiting examples of immune checkpoint biomarkers include CD27, CD28, CD40 (including CD40L), CD122, CD137, OX40, GITR/TNFRSF18, ICOS, CTLA-4, HAVCR2/TIM-3, LAG3, PD1, TIGIT, Tie-2, and gp130. In embodiments, the immune checkpoint biomarker is CD27, CD28, CD40L (soluble), CTLA-4, GITR/TNFRSF18, HAVCR2/TIM-3, LAG3, OX40, PD1, TIGIT, Tie-2, gp130, or a combination thereof.

Further exemplary biomarkers that can be detected with the method described herein include, but are not limited to, IL-1α, IL-10, IL-1RA, IL-2, IL-3, IL-4, IL-5, IL-6 IL-7, IL-8. IL-8 (HA), IL-9, IL-10, IL-12p70, IL-12/IL-23p40, IL-13, IL-15, IL-16, IL-17A, IL-17A/F, IL17-B, IL-17C, IL-17D, IL-17E/IL-25, IL-17F, IL-21, IL-22, IL-23, IL-27, IL-27p28/IL-30, IL-31, IL-33, IFNα2, IFN-β, IFN-γ, TNF-α, TNF-β, MIP-1α, MIP-1β, MIP-3α, IP-10, Eotaxin, Eotaxin-3, TARC, MCP-1, MCP-2, MCP-4, GM-CSF, G-CSF, MDC, KC/GRO. VEGF-A, VEGF-C, VEGF-D, VEGFR-1/Flt-1, E-selectin, P-selectin, thrombomodulin, ICAM-3, Procalcitonin (PCT), CD20, CD5, Flt3-L, CRP, PIGF, SAA, Tie-2, TSLP, VCAM-1, ferritin, CD147, NfL, KIM-1, I-TAC, Granzyme A, Granzyme B, and troponin (e.g., sTnI). In embodiments, one or more of the biomarkers described herein interacts with one or more viral proteins described herein. For example, SARS-CoV-2 spike protein may be capable of interacting with CD147 (also known as basigin), a receptor expressed by host cells. In embodiments, the method is a multiplexed method capable of detecting one or more of the biomarkers described herein.

In embodiments, the method detects and/or quantifies the amount of one or more inflammatory response biomarkers in a biological sample. In embodiments, the method simultaneously detects and/or quantifies the amount of IFN-γ, IL-1β, IL-2, IL-4, IL-6, IL-8, IL-10, IL-12p70, IL-13, and TNF-α in a biological sample. In embodiments, the method simultaneously detects and/or quantifies the amount of one or more chemokines in a biological sample. In embodiments, the method simultaneously detects and/or quantifies the amount of Eotaxin, Eotaxin-3, IL-8, IL-8 (HA), IP-10, MCP-1, MCP-4, MDC, MIP-1α, MIP-1β, and TARC in a biological sample. In embodiments, the method simultaneously detects and/or quantifies the amount of one or more vascular injury biomarkers in a biological sample. In embodiments, the method simultaneously detects and/or quantifies the amount of CRP, ICAM-1, SAA, and VCAM-1 in a biological sample. In embodiments, the immunoassay method simultaneously detects and/or quantifies CRP, LBP, ICAM-1, SAA, and VCAM-1 in a biological sample. In embodiments, the method simultaneously detects and/or quantifies the amount of one or more angiogenesis biomarkers in a biological sample. In embodiments, the method simultaneously detects and/or quantifies the amount of FGF (basic), PIGF, Tie-2, VEGF-A, VEGF-C, VEGF-D. VEGFR-1/Flt-1 in a biological sample. In embodiments, the method detects and/or quantifies the amount of angiopoietin-2 (Ang-2) in a biological sample.

In embodiments, the binding reagent that specifically binds the biomarker described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof.

Extracellular Vesicles

In embodiments, the biomarker is an extracellular vesicle. Extracellular vesicles, also known as EVs or exosomes, are small membrane vesicles released by most cell types. For example, virus-infected cells release EVs that can mediate further in vivo viral spread in a variety of ways and produce other pathogenic effects. For example, EVs have been shown to transfer membrane-associated viral proteins, viral cargo proteins or RNAs, indirectly assist pathogens in escaping the immune system, or inhibit an immune response. EVs can also transfer viral genes from SARS-CoV-2 infected to non-infected cells and can induce inflammation in the absence of direct viral infection.

In embodiments, detecting EVs from infected cells is used to identify reservoirs of infection. In embodiments, EV populations in a biological sample are analyzed to determine the mechanism of infection, disease prognosis, and adaptive immunity. In embodiments, an EV released from a particular cell, e.g., an immune cell, comprises one or more of the same surface marker as that cell. In embodiments, the biomarker is an EV comprising an inflammatory damage and/or a tissue damage protein as described herein, on the surface of the EV.

In embodiments, the biomarker is an EV comprising a viral protein described herein, e.g., on the surface of the EV or inside the EV. In embodiments, surface markers on an EV are used to determine the abundance of a cellular subpopulation, e.g., a subpopulation of cells infected with a virus described herein (e.g., a coronavirus such as SARS-CoV-2), or a subpopulation of cells corresponding to an immune response. In embodiments, the binding reagent binds an EV surface marker, e.g., an inflammatory damage protein, a tissue damage protein, and/or a viral protein described herein. In embodiments, multiple binding reagents are contacted with an EV, wherein at least one binding reagent binds to a host protein and at least one binding reagent binds to a viral protein, e.g., a respiratory virus protein. In embodiments, the host protein is a tissue specific surface marker, e.g., a brain, kidney, intestine, and/or respiratory tract. In embodiments, the tissue specific surface marker is a tissue that is not typically associated with the primary site of infection by the virus described herein. For example, SARS-CoV-2 has been shown to primary target the respiratory tract, and detection of EVs indicative of infected cells in other tissues, e.g., brain, kidneys, and intestine, is used to identify secondary sites of infection and/or organ damage. In embodiments, the cell type from which the EV originated is identified using a binding reagent that binds to a tissue specific surface marker. In embodiments, multiple binding reagents are contacted with an EV, wherein at least one binding reagent binds to an EV surface marker typically expressed by EVs secreted from any cell type (also referred to herein as “common” EV surface marker and includes, e.g., CD81, CD9, or CD63, known as a tetraspanins) and at least one binding reagent bind to a viral protein, e.g., a respiratory virus protein. In embodiments, the respiratory virus is a coronavirus. In embodiments, the respiratory virus is SARS-CoV-2. In embodiments, the multiple binding reagents comprise a binding reagent that binds to a tetraspanin and a binding reagent that binds to SARS-CoV-2 S protein. In embodiments, the multiple binding reagents comprise a binding reagent that binds to SARS-CoV-2 S protein, a binding reagent that binds to SARS-CoV-2 M protein, and a binding reagent that binds to SARS-CoV-2 E protein.

In embodiments, the method is a multiplexed immunoassay method capable of detecting multiple EVs. In embodiments, a multiplexed EV assay advantageously allows the same sample containing multiple EVs of interest to be assayed in one experiment, thereby reducing the amount of sample required and also decreasing sample-to-sample variability. In embodiments, a multiplexed EV assay facilitates comparison of different EVs in a sample, e.g., to determine the relative abundance of different EVs.

In embodiments, the binding reagent that specifically binds the EV biomarker described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof.

Viral Component and Biomarker Detection

In embodiments, the invention provides a method comprising simultaneously detecting a host biomarker (e.g., an antibody biomarker or inflammatory and/or tissue damage response biomarker) described herein and a viral component described herein. A method that simultaneously determines, from a single sample, whether a subject is infected by a virus (e.g., a coronavirus such as SARS-CoV-2) and assesses the subject's immune response is capable of determining the subject's disease prognosis, for example, determining whether the subject will likely have poor disease progression and increased likelihood of intensive care treatment. Thus, the method enables preparation of an early response to a potentially serious illness.

In embodiments, the method is a multiplexed immunoassay method. In embodiments, the multiplexed immunoassay method detects a viral nucleic acid, a host antibody biomarker, a host inflammatory and/or tissue damage response biomarker, or a combination thereof.

In embodiments, the multiplexed immunoassay method simultaneously detects a viral protein and a host antibody biomarker. In embodiments, the multiplexed immunoassay method detects a viral nucleic acid and a host antibody biomarker. Detection of host antibody biomarkers is described herein. In embodiments, the host antibody biomarker is capable of binding to a viral antigen from SARS-CoV-2, SARS-CoV, MERS-CoV, HcoV-OC43, HcoV-229E, HcoV-NL63, HcoV-HKU1, influenza A, influenza B, RSV, or a combination thereof. In embodiments, the host antibody biomarker is capable of binding to any of the viral antigens described herein, e.g., S (including the SARS-CoV-2 S-D614 and S-D614G variants, and any of the SARS-CoV-2 S protein variants in Tables 1A and 1B), S1, S2. S-NTD, S-ECD, S-RBD, M, E (including the SARS-CoV-2 E protein variants in Table 1A), N (including the SARS-CoV-2 N protein variants in Table 1A), F, HA, or nsp (including the SARS-CoV-2 Orf1ab and Orf8 protein variants in Table 1A). In embodiments, the host antibody biomarker is IgG. IgA, IgE, or IgM, or any subclass thereof, e.g., IgG1, IgG2, IgG3, or IgG4. In embodiments, the IgG, IgA, IgE, and/or IgM is from a human, mouse, rat, ferret, minx, bat, or combination thereof. In embodiments, the viral nucleic acid is DNA. In embodiments, the viral nucleic acid is RNA. Detection of viral nucleic acids is described herein. In embodiments, the virus is a coronavirus. In embodiments, the coronavirus is SARS-CoV-2. In embodiments, method comprises detecting a host antibody biomarker and a viral nucleic acid, wherein the host antibody biomarker is detected via a bridging serology assay. In embodiments, method comprises detecting a host antibody biomarker and a viral nucleic acid, wherein the host antibody biomarker is detected via a competitive serology assay. Bridging and competitive serology assays are further described herein.

In embodiments, the multiplexed immunoassay method simultaneously detects a host antibody biomarker and a host inflammatory and/or tissue damage response biomarker. In embodiments, the viral protein is a SARS-CoV-2 protein. In embodiments, the viral protein is SARS-CoV-2 N protein, SARS-CoV-2 S protein, or both. Detection of host inflammatory and/or tissue damage response biomarkers is described herein. In embodiments, the multiplexed simultaneously detects a SARS-CoV-2 protein and a host inflammatory and/or tissue damage response biomarker. In embodiments, the host biomarker is GM-CSF, Granzyme A, Granzyme B, IFN-α2a, IFN-β, IFN-γ, IL-1β, IL-1RA, IL-4, IL-5, IL-6, IL-7. IL-8, IL-9, IL-12p70, IP-10, I-TAC, MCP-1, MCP-2, MCP-4, MDC, MIP-1a, MIP-1β, TNF-α, VEGF-A, or a combination thereof. In embodiments, the multiplexed immunoassay method simultaneously detects: (i) one or both of SARS-CoV-2 N or S protein; and (ii) one or more of GM-CSF, Granzyme A, Granzyme B, IFN-α2a, IFN-β, IFN-γ, IL-1β, IL-1RA, IL-4, IL-5, IL-6, IL-7, IL-8, IL-9, IL-12p70, IP-10, I-TAC, MCP-1, MCP-2, MCP-4, MDC, MIP-1α, MIP-1β, TNF-α, VEGF-A. In embodiments, the immunoassay is conducted on a surface comprising multiple binding domains as depicted in FIG. 39A or 39B.

In embodiments, the multiplexed immunoassay method simultaneously detects a host antibody biomarker and a host inflammatory and/or tissue damage response biomarker. In embodiments, the host antibody biomarker binds a SARS-CoV-2 protein. In embodiments, the host antibody biomarker binds SARS-CoV-2 N protein, SARS-CoV-2 S protein, or both. Detection of host inflammatory and/or tissue damage response biomarkers is described herein. In embodiments, the multiplexed simultaneously detects a host antibody biomarker that binds a SARS-CoV-2 protein and a host inflammatory and/or tissue damage response biomarker. In embodiments, the host inflammatory and/or tissue damage response biomarker is G-CSF, GM-CSF, Granzyme A, Granzyme B, IFN-α2a, IFN-β, IFN-γ, IL-1β, IL-1RA, IL-4, IL-5, IL-6, IL-7, IL-9, IL-12p70, IP-10, I-TAC. MCP-1, MCP-2, MCP-4, MDC, MIP-1α, MIP-1β, TNF-α, VEGF-A, or a combination thereof. In embodiments, the multiplexed immunoassay method simultaneously detects: (i) one or both of a host antibody biomarker that binds SARS-CoV-2 N or a host antibody biomarker that binds SARS-CoV-2 S; and (ii) one or more of G-CSF, GM-CSF, Granzyme A. Granzyme B. IFN-α2a, IFN-β, IFN-γ, IL-1β, IL-1RA, IL-4, IL-5, IL-6, IL-7, IL-9, IL-12p70, IP-10, I-TAC, MCP-1, MCP-2, MCP-4, MDC, MIP-1α, MIP-1β, TNF-α, VEGF-A. In embodiments, the immunoassay is conducted on a surface comprising multiple binding domains as depicted in FIG. 39A or 39B.

In embodiments, the multiplexed immunoassay method detects a viral nucleic acid and a host inflammatory and/or tissue damage response biomarker. Detection of host inflammatory and/or tissue damage response biomarkers is described herein. In embodiments, the host inflammatory and/or tissue damage response biomarker is C-reactive protein (CRP), IFNα2, IFN-γ, IL-6, IL-10, MCP-1, IP-10, troponin (e.g., skeletal troponin-I (sTnI)), IL-1β, IL-2, IL-4, IL-7, granulocyte colony-stimulating factor (G-CSF), MIP-1α, TNF-α, ferritin, CD147, NfL, KIM-1, IL-8, MIP-1β, MCP-4, TARC, Flt-1, PIGF, VEGF-A, VEGF-C, ICAM-1, SAA, VCAM-1, or a combination thereof. In embodiments, the viral nucleic acid is DNA. In embodiments, the viral nucleic acid is RNA. Detection of viral nucleic acids is described herein. In embodiments, the virus is a coronavirus. In embodiments, the coronavirus is SARS-CoV-2. In embodiments, the multiplexed immunoassay method simultaneously detects a SARS-CoV-2 RNA and C-reactive protein (CRP), IFNα2, IFN-γ, IL-6, IL-10, MCP-1, IP-10, troponin (e.g., skeletal troponin-I (sTnI)), IL-1β, IL-2, IL-4, IL-7, granulocyte colony-stimulating factor (G-CSF), MIP-1α, TNF-α, ferritin. CD147, NfL, KIM-1, IL-8, MIP-1β, MCP-4, TARC, Flt-1, PIGF, VEGF-A, VEGF-C, ICAM-1, SAA, VCAM-1, or a combination thereof.

In embodiments, the multiplexed immunoassay method simultaneously detects (1) a viral component; (2) a host antibody biomarker; and (3) a host inflammatory and/or tissue damage response biomarker. In embodiments, the multiplexed immunoassay method detects a viral nucleic acid, a host antibody biomarker, and a host inflammatory and/or tissue damage response biomarker. In embodiments, the multiplexed immunoassay method detects a viral protein, a host antibody biomarker, and a host inflammatory and/or tissue damage response biomarker. In embodiments, the multiplexed immunoassay method simultaneously detects the N protein from SARS-CoV-2; host biomarkers IL-6 and IFN-γ; and an antibody biomarker against the SARS-CoV-2 S protein or subunit thereof via a serology assay described herein, e.g., a classical, bridging, or competitive serology assay. In embodiments, the multiplexed immunoassay method simultaneously detects the N protein from SARS-CoV-2; host biomarkers IL-6, IFN-γ, and IFNα2; and an antibody biomarker against the SARS-CoV-2 S protein or subunit thereof via a serology assay described herein, e.g., a classical, bridging, or competitive serology assay. In embodiments, the multiplexed immunoassay method simultaneously detects the N protein from SARS-CoV-2; host biomarkers IL-6 and IFN-γ; and an antibody biomarker against the SARS-CoV-2 S protein (or subunit thereof) via an ACE2 competitive serology assay, as described herein. In embodiments, the multiplexed immunoassay method simultaneously detects the N protein from SARS-CoV-2; host biomarkers IL-6 and IFN-γ; and an antibody biomarker against the SARS-CoV-2 S-RBD via a bridging serology assay as described herein. In embodiments, the multiplexed immunoassay method simultaneously detects the N protein from SARS-CoV-2; host biomarkers IL-6, IFN-γ, and IFNα2; and an antibody biomarker against the SARS-CoV-2 S protein (or subunit thereof) via an ACE2 competitive serology assay, as described herein. In embodiments, the multiplexed immunoassay method simultaneously detects the N protein from SARS-CoV-2; host biomarkers IL-6. IFN-γ, and IFNα2; and an antibody biomarker against the SARS-CoV-2 S-RBD via a bridging serology assay as described herein.

Detection of viral nucleic acids and viral proteins are provided by the invention. In embodiments, the viral nucleic acid is DNA. In embodiments, the viral nucleic acid is RNA. Detection of host antibody biomarkers is provided by the invention. In embodiments, the host antibody biomarker is capable of binding to a viral antigen from SARS-CoV-2. SARS-CoV, MERS-CoV, HcoV-OC43, HcoV-229E, HcoV-NL63, HcoV-HKU1, influenza A, influenza B, RSV, or a combination thereof. In embodiments, the host antibody biomarker is capable of binding to any of the viral antigens described herein, e.g., S (including the SARS-CoV-2 S-D614 and S-D614G variants, and any of the SARS-CoV-2 S protein variants in Tables 1A and 1B), S1, S2. S-NTD, S-ECD, S-RBD, M, E (including the SARS-CoV-2 E protein variants in Table 1A), N (including the SARS-CoV-2 N protein variants in Table 1A), F. HA, or nsp (including the SARS-CoV-2 Orf1ab and Orf8 protein variants in Table 1A). In embodiments, the host antibody biomarker is IgG, IgA, IgE, or IgM, or any subclass thereof, e.g., IgG1, IgG2, IgG3, or IgG4. In embodiments, the IgG, IgA, IgE, and/or IgM is from a human, mouse, rat, ferret, minx, bat, or combination thereof. In embodiments, the host antibody biomarker is detected via a bridging serology assay. In embodiments, the host antibody biomarker is detected via a competitive serology assay. Bridging and competitive serology assays are further disclosed herein. The invention provides detection of host inflammatory and/or tissue damage response biomarkers. In embodiments, the host inflammatory and/or tissue damage response biomarker is C-reactive protein (CRP), IFNα2, IFN-γ, IL-6, IL-10, MCP-1, IP-10, troponin (e.g., skeletal troponin-I (sTnI)), IL-1β, IL-2, IL-4, IL-7, granulocyte colony-stimulating factor (G-CSF), MIP-1α, TNF-α, ferritin, CD147, NfL, KIM-1, IL-8, MIP-1β, MCP-4, TARC, Fit-1, PIGF, VEGF-A, VEGF-C, ICAM-1, SAA, VCAM-1, Ang-2, or a combination thereof. In embodiments, the virus is a coronavirus. In embodiments, the coronavirus is SARS-CoV-2. In embodiments, the multiplexed immunoassay method simultaneously detects a SARS-CoV-2 RNA; an antibody that binds to one or more of SARS-CoV-2 S (including the SARS-CoV-2 S-D614 and S-D614G variants), S1, S2, S-NTD, S-ECD, S-RBD, M, E, N, F, HA, or nsp; and C-reactive protein (CRP), IFNα2, IFN-γ, IL-6, IL-10, MCP-1, IP-10, troponin (e.g., skeletal troponin-I (sTnI)), IL-1β, IL-2, IL-4, IL-7, granulocyte colony-stimulating factor (G-CSF), MIP-1α, TNF-α, ferritin, CD147, NfL, KIM-1, IL-8, MIP-1β, MCP-4, TARC, Flt-1, PIGF, VEGF-A, VEGF-C, ICAM-1, SAA, VCAM-1. Ang-2, or a combination thereof. In embodiments, the antibody is detected using a bridging serology assay. In embodiments, the antibody is detected using a competitive serology assay.

In embodiments, a subject's infection status, disease progression, prognosis, or combination thereof is assessed by simultaneously detecting (1) a viral component, (2) a host antibody biomarker, and (3) a host inflammatory and/or tissue damage response biomarker as described herein. For example, Table 2 provides exemplary outcomes and assessments based on the combined detection for diagnosis and prognosis of COVID-19, the disease caused by SARS-CoV-2 infection.

TABLE 2
Exemplary Scenarios and Predicted Outcomes

| | | | Inflammatory/ | |
|---|---|---|---|---|
| | | | Tissue | |
| | SARS- | | Damage | COVID-19 |
| | COV-2 | Host | Response | Diagnosis, |
| Scenario | Viral | Antibody | (e.g., cytokine/ | Disease |
| # | Component | (Serology) | chemokine) | Stage |
| 1 | + | − | Normal | Early stage; |
| | | | | asymptomatic |
| 2 | − | + | Normal | Convalescent; |
| | | | | recovered |
| 3 | + | + | Elevated | Late stage, active |
| | | | | infection |
| 4 | − | − | Elevated | Non-COVID-19 |
| | | | | inflammation |
| 5 | + | +/− | Highly | Active acute stage |
| | | | Elevated | infection. May |
| | | | | require hospital |
| | | | | admission |
| 6 | − | − | Normal | No exposure to |
| | | | | COVID-19 |

### Samples and Assay Devices

In embodiments, the viruses, viral components, and/or biomarkers described herein are measured in a biological sample. In embodiments, the biological sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, a respiratory sample, bronchial/bronchoalveolar lavage, saliva, mucus, oropharyngeal swab, sputum, endotracheal aspirate, pharyngeal/nasal swab, throat swab, amniotic fluid, nasal secretions, nasopharyngeal wash or aspirate, nasal mid-turbinate swab, vaginal secretions, a surface biopsy, sperm, semen/seminal fluid, wound secretions and excretions, ear secretions or discharge, or an extraction, purification therefrom, or dilution thereof. In embodiments, the biological sample is diluted such that the assay signal is within the upper and lower detection limits of the assay. In embodiments, the biological sample is diluted to achieve a desired assay sensitivity. Further exemplary biological samples include but are not limited to physiological samples, samples containing suspensions of cells such as mucosal swabs, tissue aspirates, endotracheal aspirates, tissue homogenates, cell cultures, and cell culture supernatants. In embodiments, the biological sample is a respiratory sample obtained from the respiratory tract of a subject. Examples of respiratory samples include, but are not limited to, bronchial/bronchoalveolar lavage, saliva, mucus, endotracheal aspirate, sputum, nasopharyngeal/nasal swab, throat swab, oropharyngeal swab and the like. In embodiments, the biological sample is whole blood, serum, plasma, cerebrospinal fluid (CSF), urine, saliva, sputum, endotracheal aspirate, nasopharyngeal/nasal swab, bronchoalveolar lavage, or an extraction or purification therefrom, or dilution thereof. In embodiments, the biological sample is serum or plasma. In embodiments, the plasma is in EDTA, heparin, or citrate. In embodiments, the biological sample is saliva. In embodiments, the biological sample is endotracheal aspirate. In embodiments, the biological sample is a nasal swab. In embodiments, the virus, viral component, and/or biomarkers described herein have substantially levels in the saliva or endotracheal aspirate of a subject. In embodiments, the virus, viral components, and/or biomarkers described herein are present in higher amounts in certain bodily fluids (e.g., saliva) compared to others (e.g., throat swab). In embodiments, certain antibody biomarker levels, e.g., IgG (including subclasses thereof) and IgA, are substantially similar in blood and saliva of a subject.

In embodiments, the biological sample is from an animal. In embodiments, the biological sample from an animal is useful for animal model studies, e.g., for vaccine and/or drug research and development, and/or to better understand disease progression and infection lethality. Exemplary animals that are useful for animal model studies include, but are not limited to, mouse, rat, rabbit, pig, primate such as monkey, and the like.

In embodiments, the biological sample is from a human or an animal subject. In embodiments, the subject is susceptible or suspected to be susceptible to infection by the viruses described herein. In embodiments, the subject is known or suspected to transmit the viruses described herein. Virus transmission may occur among the same species (e.g., human-to-human) or inter-species (e.g., bat-to-human). Non-limiting examples of animal subjects include domestic animals, such as dog, cat, horse, goat, sheep, donkey, pig, cow, chicken, duck, rabbit, gerbil, hamster, guinea pig, and the like; non-human primates (NHP) such as macaque, baboon, marmoset, gorilla, orangutan, chimpanzee, monkey, and the like; big cats such as tiger, lion, puma, leopard, snow leopard, and the like; and other mammals such as bats and pangolins. In embodiments, the biological sample is from a human, a mouse, a rat, a ferret, a minx, or a bat. In embodiments, the subject is a host that has been exposed to and/or infected by a virus as described herein. In embodiments, the biological ample comprises a plasma (e.g., in EDTA, heparin, or citrate) sample from a subject. In embodiments, the biological sample comprises a serum sample from a subject. In embodiments, the biological sample is from a healthy subject. In embodiments, the biological sample is from a subject known to never have been exposed to a virus described herein. In embodiments, the biological sample is from a subject known to be immune to a virus described herein. In embodiments, the biological sample is from a subject known to be infected with a virus described herein. In embodiments, the biological sample is from a subject suspected of having been exposed to a virus described herein. In embodiments, the biological sample is from a subject at risk of being exposed to a virus described herein. In embodiments, the virus is a coronavirus. In embodiments, the virus is SARS-CoV-2.

In embodiments, the sample is an environmental sample. In embodiments, the environmental sample is aqueous, including but not limited to, fresh water, drinking water, marine water, reclaimed water, treated water, desalinated water, sewage, wastewater, surface water, ground water, runoff, aquifers, lakes, rivers, streams, oceans, and other natural or non-natural bodies of water. In embodiments, the aqueous sample contains bodily solids or fluids (e.g., feces or urine) from subjects who have been exposed to or infected with a virus herein (e.g., a coronavirus such as SARS-CoV-2). In embodiments, the environmental sample is from a air filtration device, e.g., air filters in a healthcare or long-term care facility or other communal places of gathering. Detection of a virus described herein (e.g., a coronavirus such as SARS-CoV-2) in an environmental sample can provide early identification and/or tracing of an outbreak or potential outbreak, thereby allowing a more prompt and robust response. Moreover, detection of a biomarker, e.g., one or more antibody biomarkers that specifically binds a viral antigen (e.g., from a coronavirus such as SARS-CoV-2) in an environmental sample can provide an estimation of the percentage of a population with detectable antibodies against the virus (i.e., seroconversion), which is useful for epidemiology studies.

In embodiments, the sample comprises wastewater. As used herein, “wastewater” includes any water that has been contaminated by human use, including any combination of domestic, industrial, commercial, or agricultural activities, surface runoff or stormwater, and any sewer inflow or sewer infiltration. In embodiments, the sample comprises wastewater from a sewage system. Wastewater-based epidemiology (WBE) can be used for surveillance and genotyping of viral infections, including, e.g., norovirus (Kazama et al., Appl Environ Microbial 83(9):e03406-03416 (2017)). WBE can lead to detection of disease several days before a significant portion of a population becomes symptomatic.

SARS-CoV-2 RNA has been detected in wastewater using RT-PCR (see. e.g., Green et al., medRxiv pre-print doi:10.1101/2020.05.21.20109181 (21 May 2020); and Medema et al., Environ Sci Technol Lett 7(7):511-516 (2020); and Ahmed et al., Sci Total Environ 728:138764 (2020)). Wastewater samples are useful for detection of SARS-CoV-2, as the virus can be detected in feces as early as one day after onset of disease and can persist for up to 22 days, which is longer than the typical time period for nasopharyngeal samples. In embodiments, the invention provides methods for detecting SARS-CoV-2 proteins in wastewater. In embodiments, the invention provides a method for detecting SARS-CoV-2 in a wastewater sample, comprising: a) contacting the wastewater sample with a binding reagent that specifically binds a SARS-CoV-2 protein; b) forming a binding complex comprising the binding reagent and the SARS-CoV-2 protein; and c) detecting the binding complex, thereby detecting SARS-CoV-2 in the wastewater sample. In embodiments, the SARS-CoV-2 protein is S protein, N protein, E protein, M protein, or a combination thereof. In embodiments, the SARS-CoV-2 protein is N protein. In embodiments, the SARS-CoV-2 protein is an S protein. In embodiments, the SARS-CoV-2 S protein comprises a mutation as shown in Tables 1A and 1B. In embodiments, the SARS-CoV-2 N protein comprises a mutation as shown in Table 1A.

Wastewater samples are also useful for determining the viral strain, i.e., the genotype, of SARS-CoV-2 in a population. SARS-CoV-2 strains are further described herein and include. e.g., the L strain and the S strain, which differ at genome locations 8782 and 28144; and the S-D614 strain and the S-D614G strain, which differ by a single polynucleotide at genome location 23403, and the strains described in Table 1A, e.g., strains B.1.1.7, 501Y.V2, P.1, and Cal.20C. In embodiments, the invention provides a method for detecting SARS-CoV-2 nucleic acid in a wastewater sample, comprising: a) contacting the wastewater sample with a binding reagent that specifically binds a SARS-CoV-2 nucleic acid; b) forming a binding complex comprising the binding reagent and the SARS-CoV-2 nucleic acid; and c) detecting the binding complex, thereby detecting the SARS-CoV-2 nucleic acid in the wastewater sample. In embodiments, the SARS-CoV-2 nucleic acid comprises a SARS-CoV-2 single nucleotide polymorphism (SNPs) or mutation as described herein, e.g., in Tables 1A and 1C. In embodiments, the method is a multiplexed method that simultaneously detects one or more, two or more, three or more, four or more, five or more, six or more, seven or more, eight or more, nine or more, or ten or more SARS-CoV-2 SNPs. Methods of detecting SNPs in viral nucleic acids, e.g., SARS-CoV-2 RNA, are provided herein.

Challenges associated with virus detection in wastewater samples include the inherently labile nature of RNA and its degradation by RNAse enzymes present in wastewater and the difficulty in determining appropriate threshold levels of viral load in the sample, which can vary based on factors such as prevalence of the virus in a population, strain and severity of the viral infection, and other environmental and socio-economic factors. Viral proteins and genetic materials (e.g., RNA) from human waste are greatly diluted in the sewage system, and further dilution can occur when household sewage is mixed with storm water and wastewater from businesses and public areas. Moreover, seasonal variation can impact water consumption, and ambient temperature can affect the stability of the viral proteins and/or genetic materials. Presence of household and commercial disinfectants and detergents can also impact the rate of degradation of viral proteins and/or genetic materials. These factors contribute to a large discrepancy in measured concentrations of virus in wastewater samples. For example, the range of viral load in feces of SARS-CoV-2 infected patients can vary from 103 to 10⁷ copies/mL, and the concentrations of SARS-CoV-2 genetic material in wastewater ranges from 0.02 to 200000 copies/mL (see, e.g., Michael-Kordatoua et al., J Environ Chem Eng 8(5):104306 (2020); Foladori et al., Sci Total Environ 743:140444 (2020)). Such variation in the viral load of SARS-CoV-2 in human feces is further attributed to additional factors such as the severity and stage of viral infection and whether the patient presented with gastrointestinal symptoms of viral infection (e.g., diarrhea). For example, the reported percentage of COVID-19 patients with detectable SARS-CoV-2 genetic material in stool varies from 15% to 80% of total COVID-19 patients.

In embodiments, levels of IgA, IgG, and/or IgM in wastewater samples are used as controls for normalizing the detected amount of viral protein and/or genetic material (e.g., RNA) in the wastewater sample. IgA and IgG are present in human intestines and secreted at concentrations of approximately 1000 μg/g and 20 μg/g, respective (see, e.g., Lin et al., J Transl Med 16:359 (2018)). In embodiments, IgA is detectable in a wastewater sample at greater than or about 0.01 μg/mL, greater than or about 0.05 μg/mL, greater than or about 0.1 μg/mL, greater than or about 0.2 μg/mL, or greater than or about 0.3 μg/mL. In embodiments, the level of a human housekeeping protein is used as a control for normalizing the detected amount of viral protein and/or genetic material (e.g., RNA) in the wastewater sample. As used herein, a “housekeeping protein” refers to a typically constitutively expressed protein that is required for the maintenance of basic cellular functions, and is expressed in all cells of an organism (e.g., a human) under normal and pathophysiological conditions. In embodiments, the housekeeping protein is ribosomal protein 4S, glyceraldehyde-3-phosphate dehydrogenase (GADPH), β-actin, β-tubulin, or a combination thereof. In embodiments, the virus is a coronavirus. In embodiments, the virus is SARS-CoV-2.

In embodiments, the invention provides a method of measuring the amount of a virus in a wastewater sample, comprising: a) measuring the amount of a viral component in the wastewater sample using an immunoassay described herein, b) measuring the amount of control (e.g., IgA, IgG, IgM and/or a housekeeping protein) in the wastewater sample; and c) normalizing the detected amount of viral component to the control, thereby measuring the amount of the virus in the wastewater sample. In embodiments, the control comprises IgA, IgG, and IgM. In embodiments, the virus is a coronavirus. In embodiments, the virus is SARS-CoV-2. In embodiments, the viral component is a viral protein. In embodiments, the viral component is DNA or RNA. In embodiments, the viral component is SARS-CoV-2 S protein. SARS-CoV-2 N protein, SARS-CoV-2 M protein, SARS-CoV-2 E protein, or a combination thereof. In embodiments, the viral component is SARS-CoV-2 RNA. In embodiments, the SARS-CoV-2 RNA comprises a SARS-CoV-2 single nucleotide polymorphism (SNPs) as described herein, e.g., in Tables 1A and 1C.

In embodiments, the invention provides a method of detecting a biomarker that binds a SARS-CoV-2 antigen in a wastewater sample. In embodiments, the biomarker is an antibody biomarker. Methods of detecting antibody biomarkers, e.g., serology assays, are described herein. In embodiments, the method of detecting a biomarker that binds a SARS-CoV-2 antigen in a wastewater sample simultaneously detects and/or quantifies one or more biomarkers in the wastewater sample that binds to: an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, and/or an F protein from RSV. In embodiments, the S protein is a subunit, domain, or fragment thereof, e.g., S1, S2, S-NTD, S-ECD, or S-RBD. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments where the sample comprises a liquid (e.g., endotracheal aspirate, saliva, blood, serum, plasma and the like), the sample is about 0.05 mL to about 50 mL, about 0.1 mL to about 10 mL, about 0.2 mL to about 5 mL, or about 0.3 mL to about 3 mL. In embodiments where the sample is solid or semi-solid (e.g., a swab such as a nasopharyngeal swab or oropharyngeal swab, mucus, sputum and the like), the sample is provided into a storage liquid of about 0.05 mL to about 50 mL, about 0.1 mL to about 10 mL, about 0.2 mL to about 5 mL, or about 0.3 mL to about 3 mL. In embodiments, the storage liquid is Viral Transport Medium (VTM), Amies transport medium, or sterile saline. In embodiments, the storage liquid comprises a substance for stabilizing nucleic acids, e.g., EDTA. In embodiments, the storage liquid comprises a reagent for inactivating live virus as described herein.

In embodiments, the sample comprises saliva. In embodiments, the invention provides a method of identifying a saliva sample in which the viral component and/or biomarker of interest has degraded, i.e., a low quality saliva sample. In embodiments, a low quality saliva sample is not suitable for the assays described herein. In embodiments, a low quality saliva sample comprises low levels of IgA as compared to a freshly obtained sample and/or a threshold antibody level. In embodiments, the threshold antibody level is determined based on the average of an aggregate of samples. In embodiments, a low quality saliva sample comprises low levels of antibodies against circulating coronaviruses (e.g., HCoV-NL63, HCoV-HKU1, HCoV-229E, and/or HCoV-OC43) as compared to a freshly obtained sample and/or a threshold antibody level. In embodiments, identifying the low quality saliva sample comprises determining the IgA level in a sample and, if the sample has low IgA levels as compared to a freshly isolated control sample and/or as compared to a threshold antibody level, identifying the sample as a low quality saliva sample. In embodiments, identifying the low quality saliva sample comprises determining the levels of antibodies against one or more circulating coronaviruses in a sample and, if the sample has low antibody levels against the one or more circulating coronaviruses as compared to a freshly isolated control sample and/or a threshold antibody level, identifying the sample as a low quality saliva sample.

In embodiments, the sample comprises an extracellular vesicle. As described herein, extracellular vesicles (also known as EVs or exosomes) are small membrane vesicles released by most cell types, including immune cells and infected cells (e.g., by a respiratory virus described herein such as SARS-CoV-2). The release and subsequent uptake of EVs is a method of cell-to-cell communication and has a role in the regulation of many physiological and pathological processes. EVs contain a wide variety of signaling molecules, including but not limited to surface-bound and cytosolic proteins, lipids, mRNA, and miRNA, and in embodiments the identity and concentration of these species in each EV is used to deduce its cellular origin and function. Thus, in embodiments, genomic or proteomic profiling of a subject's total EV population provides valuable prognostic information for various pathological conditions, including infections, e.g., by a virus described herein. Detection and analysis of EVs are further described, e.g., in WO 2019/222708 and WO 2020/086751.

In embodiments, the sample is pretreated prior to being subjected to the methods provided herein. In embodiments, the sample is pretreated prior to being handled by, processed by, or in contact with laboratory and/or clinical personnel. In embodiments, pretreating the sample comprises subjecting the sample to conditions sufficient to inactivate live virus in the sample. Inactivation of live virus that may be present in the sample reduces the risk of infection of the laboratory and/or clinical personnel handling and/or processing the sample, e.g., by performing the methods described herein on the sample. In embodiments, pretreating the sample comprises heating the sample to at least 55° C., at least 56° C., at least 57° C., at least 58° C., at least 59° C., at least 60° C., at least 65° C., at least 70° C., at least 75° C., at least 80° C., at least 85° C., at least 90° C., at least 95° C., or at least 100° C. In embodiments, the sample is heated for about 10 minutes to about 4 hours, about 20 minutes to about 2 hours, or about 30 minutes to about 1 hour. In embodiments, the sample is heated to about 65° C. for at least 10 minutes. In embodiments, the sample is heated to about 65° C. for at least 30 minutes. In embodiments, the sample is heated to about 58° C. for at least 1 hour.

In embodiments, pretreating the sample comprises contacting the sample with an inactivation reagent. In embodiments, the inactivation reagent comprises a detergent, a chaotropic agent, a fixative, or a combination thereof. Non-limiting examples of detergents include sodium dodecyl sulfate and TRITON™ X-100. Non-limiting examples of chaotropic agents include guanidium thiocyanate, guanidium isothiocyanate, and guanidium hydrochloride. Non-limiting examples of fixatives include formaldehyde, formalin, paraformaldehyde, and glutaraldehyde. In embodiments, pretreating the sample comprises subjecting the sample to UV or gamma irradiation. In embodiments, pretreating the sample comprises subjecting the sample to a highly alkaline (e.g., above pH 10, above pH 11, or above pH 12) condition. In embodiments, pretreating the sample comprises subjecting the sample to a highly acidic (e.g., below pH 4, below pH 3, below pH 2) condition. Additional methods of pretreating samples, e.g., containing the viruses described herein, is further discussed in Bain et al., Curr Protoc Cytometry 93:e77 (2020).

In embodiments, the sample comprises a viral nucleic acid. In embodiments, the sample comprising the viral nucleic acid is pretreated with a reagent that stabilizes and/or prevents degradation of the viral nucleic acid. In embodiments, the pretreating comprises removing and/or inhibiting activity of a nuclease, e.g., an RNase, in the sample. In embodiments, the viral nucleic acid is SARS-CoV-2 RNA.

In embodiments, the sample comprises an RT-PCR product. In embodiments, the RT-PCR product comprises a cDNA that is generated from a viral RNA. In embodiments, the sample comprising the RT-PCR product is pretreated to remove the viral RNA and/or a reagent used in the RT-PCR. In embodiments, the pretreating comprises contacting the sample with RNase. In embodiments, the pretreating comprises heating the sample, e.g., as described herein. In embodiments, the viral RNA is SARS-CoV-2 RNA.

In embodiments, the sample is pretreated immediately after being collected, e.g., from a subject described herein. Sample collection methods are provided herein. In embodiments, the sample is pretreated while being transported to a facility. e.g., a laboratory, for processing and analyzing the sample, e.g. using the methods described herein. In embodiments, the sample is pretreated after arrival at a facility, e.g., a laboratory, for processing and analyzing the sample, e.g. using the methods described herein. In embodiments, the sample is pretreated prior to being stored. In embodiments, the sample is stored prior to processing and analysis. e.g. using the methods described herein. In embodiments, the sample is stored at about −80° C. to about 30° C., about −70° C. to about 25° C., about −60° C. to about 20° C., about −20° C. to about 15° C., about 0° C. to about 10° C., about 2° C. to about 8° C., or about 4° C. to about 12° C. Methods and conditions for storing the samples described herein are known to one of ordinary skill in the art.

As used herein, the term “exposure,” in the context of a subject being exposed to a virus, refers to the introduction of a virus into the subject's body. “Exposure” does not imply any particular amount of virus; introduction of a single viral particle into the subject's body can be referred to herein as an “exposure” to the virus. As used herein, the term “infection,” in the context of a subject being infected with a virus, means that the virus has penetrated a host cell and has begun to replicate, assemble, and release new viruses from the host cell. The term “infection” can also be used to refer to an illness or condition caused by a virus, e.g., respiratory tract infection as described herein.

In embodiments, the virus, viral component, and/or biomarker are detectable in a subject immediately (e.g., within seconds) after the subject is exposed to the virus and/or infected with the virus. In embodiments, the virus, viral component, and/or biomarker are detectable in a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is exposed to the virus and/or infected with the virus. In embodiments, the virus, viral component, and/or biomarker are detectable in a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is exposed to the virus and/or infected with the virus. Different biomarkers, e.g., antibody biomarkers or inflammatory or tissue damage response biomarkers, in the same subject may have a varying magnitude of change in response to virus exposure and/or infection, for example, depending on whether the biomarker is an acute response biomarker or a biomarker related to a long-term effect. For some viral infections, the antibody biomarker IgG typically plateaus after 10 days of disease onset and persist (e.g., potentially signifying longer-term immunity); the antibody biomarkers IgA and IgM are detectable within 6 days of disease onset, peak around 10 days, and diminish after approximately 14 days (e.g., as part of the initial infection response). Different viruses can trigger biomarker responses at different times. For example, antibodies to SARS-CoV-2 may not be consistently detected in a subject until about three weeks after infection, which is longer than the typical timing for other types of viral infections. The timing of producing the same biomarker type, e.g., IgM or IgG antibody, can also vary widely among different subjects. Thus, in embodiments, the methods for multiplexed assays for a combination of biomarkers disclosed herein includes a determination or consideration of the response timing of each of the biomarkers.

In embodiments, the biological sample is obtained from a subject who has not been exposed to the virus. In embodiments, the biological sample is obtained from a subject immediately (e.g., within seconds) after the subject is known or suspected to be exposed to the virus. In embodiments, the biological sample is obtained from a subject within about 5 minutes to about 1 year, about 1 hour to about 9 months, about 6 hours to about 6 months, about 12 hours to about 90 days, 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is known or suspected to be exposed to the virus. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is known or suspected to be exposed to the virus.

In embodiments, the biological sample is obtained from a subject prior to the subject showing any symptoms of a viral infection. In embodiments, the biological sample is obtained from a subject immediately (e.g., within seconds) after the subject begins to show symptoms of a viral infection. In embodiments, the biological sample is obtained from a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject begins to show symptoms of a viral infection. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject begins to show symptoms of a viral infection. Symptoms of a viral infection are described herein and include, e.g., cough, shortness of breath, fever, and fatigue.

In embodiments, the biological sample is obtained from a subject after the subject is diagnosed with a viral infection. As described herein, the SARS-CoV-2 virus can cause post-acute COVID-19 syndrome, with certain symptoms persisting weeks or months after the initial illness period. In embodiments, the biological sample is obtained from a subject after about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 9 months, about 1 year, about 2 years, about 3 years, about 4 years, about 5 years, about 6 years, about 7 years, about 8 years, about 9 years, about 10 years, or more than 10 years after the subject is diagnosed with the viral infection.

In embodiments, the biological sample is obtained from a subject prior to the subject being administered with a vaccine or a treatment for the virus described herein. In embodiments, the biological sample is obtained from a subject immediately (e.g., within seconds) after a vaccine or a treatment is administered to the subject. In embodiments, the biological sample is obtained from a subject within about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after a vaccine or a treatment is administered to the subject. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after a vaccine or a treatment is administered to the subject.

Sample Pooling

Samples may be obtained from a single source described herein, or may contain a mixture from two or more sources, e.g., pooled from one or more individuals who may have been exposed to or infected by a particular virus in a similar manner. For example, the individuals may live or have lived in the same household, visited the same location(s), and/or associated with the same people. In embodiments, samples are pooled from two or more, three or more, four or more, five or more, six or more, seven or more, eight or more, nine or more, ten or more, 15 or more, 20 or more, 25 or more, 30 or more, 40 or more, 50 or more, 100 or more, 150 or more, 200 or more, 300 or more, 400 or more, 500 or more, 1000 or more, 5000 or more, or 10000 or more individuals. For example, a “negative” result for an active viral infection from a pooled sample indicates that none of the individuals from the pooled sample have an active infection, which can significantly reduce the number of tests needed to test every individual in a population. In embodiments, the sample comprises a respiratory sample, e.g., bronchial/bronchoalveolar lavage, saliva, mucus, oropharyngeal swab, sputum, endotracheal aspirate, pharyngeal/nasal swab, throat swab, nasal secretion, or combination thereof. In embodiments, the sample comprises saliva. In embodiments, the sample comprises blood. In embodiments, the sample comprises serum or plasma. In embodiments, the virus is a coronavirus. In embodiments, the virus is SARS-CoV-2. In embodiments, a “positive” result for an active viral infection in the pooled sample prompts or indicates a need for further testing using the methods and/or kits provided by the invention of individual samples comprised in the pool of samples.

In embodiments, the pooled sample is subjected to a single laver pooling strategy. A “single layer pooling strategy,” as used herein, refers to testing a pooled sample, and if the result of the pooled sample is “positive” for an active viral infection, each individual sample comprised in the pooled sample is then individually tested, e.g., using the methods and/or kits provided in the invention. In embodiments, the pooled sample is subjected to a multi-layer pooling strategy, e.g., a two-layer pooling strategy. In a “multi-layer pooling strategy,” a pooled sample containing n number of individual sample is tested in a first round, and if the result of the first round is “positive” for an active viral infection, then the pooled sample is divided into smaller pools, e.g., wherein each smaller pool comprises a number of individual samples equal to the square root of n, and re-tested in a second round. The smaller pool(s) with the “positive” results can be further divided into even smaller pools for one or more additional rounds of testing until the positive individual samples are identified. In an exemplary two-layer pooling strategy, a pooled sample containing 100 individual samples is tested in a first round, and if the pooled sample is tested to be “positive” for an active viral infection, then the pooled sample is divided into pools containing 10 individual samples. Each individual sample comprised in any 10-sample pools that tested “positive” are then tested.

In embodiments, the invention provides a method for determining the number of individual samples to be included in a pooled sample. In embodiments, the number of individual samples included in a pooled sample is based on disease prevalence in a population. For example, if disease prevalence is high, the likelihood of a pooled sample, containing a large number of individual samples, testing “positive” is also high, which reduces the benefits of testing pooled samples because additional tests are required to determine the positive individual samples. FIGS. 36A and 36B illustrate examples of the total number of tests needed for a population of 50,000 individuals using different pooled sample sizes, wherein the disease prevalence in the population varies from 0.001% to 100%, when using a single-layer pooling strategy (FIG. 36A) or a two-layer pooling strategy (FIG. 36B). As shown in FIG. 36A, a pool size of 100 individual samples uses the lowest number of tests overall when the disease prevalence is 0.032% or lower. When disease prevalence is 0.32%, a pool size of 30 individual samples uses the lowest number of tests overall. When disease prevalence is 1% or higher, a pool size of 10 individual samples should be used. As shown in FIG. 36B, which shows a two-layer pooling strategy, the overall number of tests needed is lower compared with the single-layer pooling strategy when using a large pool size (e.g., 100 individual samples) at low disease prevalence (e.g., 0.32% or lower). While the optimal pool size for each depicted disease prevalence rate is similar for single-layer and two-layer pooling strategies, the two-layered pooling strategy imposes a smaller penalty for using a large pool with high prevalence, as compared to the single-layered pooling strategy.

FIG. 37 illustrates a further example of dynamically selecting between three testing approaches based on disease prevalence: (1) pool size of 100 individual samples with a two-layer pooling strategy (filled circles); (2) pool size of 10 individual samples with a single-layer pooling strategy (open circles); and (3) no pooling (dashed line). The total number of tests needed for a population of 50,000 individuals is presented as a function of disease prevalence in the population. As shown in FIG. 37, at low prevalence (i.e., less than 3%), the two-layer pooling strategy provides the highest efficiency. At 3% prevalence, the two-layer pooling strategy provides no advantage (i.e., reduction in total number of tests needed) as compared with the single-layer pooling strategy, and thus, it may be more efficient to use pools of 10 individual samples with a single-layer pooling strategy. Finally, above 20% prevalence, testing all individual samples without pooling is the most efficient approach.

In embodiments, each individual sample is about 0.1 mL to about 10 mL, about 0.2 mL to about 5 mL, or about 0.3 mL to about 3 mL. In embodiments, about 1%, about 2%, about 3%, about 4%, about 5%, about 10%, or about 20% of the total volume of each individual sample is added to the pooled sample. In embodiments, about 1 μL to about 100 μL, about 5 μL to about 50 μL, or about 10 μL to about 20 μL of each individual sample is added to the pooled sample. In embodiments, the amount of each individual sample not added to the pooled sample is sufficient for one or more additional rounds of testing (e.g., in a multi-layered pooling strategy as described herein).

An exemplary approach for performing a two-layered pooling strategy in a 96-well plate is illustrated in FIG. 38. Individual samples are provided in 8×10 blocks of wells (e.g., columns 3 to 12 of a 96-well plate). The first layer comprises a pooled sample size of 80. The second layer comprises a pooled sample size of 10. In Plate Format 1 (PF1) of FIG. 38, the individual samples are contained in the 80 wells from rows A-H and columns 3-12. The individual samples in PF1 are combined according to Plate Format 2 (PF2) of FIG. 38 to create pooled samples containing 10 individual samples. Finally, the pooled samples of PF2 are further combined according to Plate Format 3 (PF3) of FIG. 38 to create pooled samples containing 80 individual samples. In embodiments, the plate comprises a barcode for identification of the samples contained therein.

Collection and Assay Devices

In embodiments, the biological sample is a liquid sample. In embodiments, the biological sample is in contact with a sample collection device. In embodiments, the sample collection device is an applicator stick. In embodiments, the sample collection device comprises an elongated handle (e.g., a rod or a rectangular prism) and a sample collection head configured to collect sample from a biological tissue (e.g., from a subject's nasal or oral cavity) or a surface. In embodiments, the sample collection head comprises an absorbent material (e.g., cotton) or a scraping blade. In embodiments, the sample collection device is a swab. In embodiments, the sample collection device is a tissue scraper.

In embodiments, the sample collection device or the liquid sample is contacted with an assay cartridge. Advantages of an assay cartridge-based method include portability and efficiency, allowing simple and rapid point-of-care diagnosis. In embodiments, the assay cartridge comprises a sample chamber and a detector. In embodiments, the assay cartridge comprises a flow cell, e.g., a microfluidic flow cell. In embodiments, the sample collection device or the liquid sample is provided to the sample chamber, and the assay cartridge is then provided to an assay cartridge reader. In embodiments, the method described herein is performed by the assay cartridge reader. In embodiments, an assay is performed in an assay cartridge, wherein the assay cartridge: moves a metered amount of sample to a first region of the assay cartridge comprising dried reagents (e.g., binding reagent and/or detection reagent as described here), thereby reconstituting the dried reagents; incubates the sample with the reconstituted reagents, thereby forming a binding complex comprising the target analyte (e.g., virus, viral component, or biomarker as described herein); mixes the incubated sample and reconstituted reagents to a second region of the assay cartridge, wherein the second region is a surface capable of immobilizing the binding reagent (e.g., via a targeting agent and targeting agent complement interaction as described herein), thereby immobilizing the binding complex on the surface; washes the surface with a wash solution to remove unbound components; and/or detects and/or quantifies the amount of immobilized binding complex on the surface. In embodiments of this method, a user provides a sample to the assay cartridge by a sample port. In embodiments, performing the method in an assay cartridge reduces incubation time (e.g., of the samples with the binding reagent, or the sample and binding reagent with the detection reagent) while minimizing reduction in signal (e.g., by increasing the concentration of one or more assay components). In embodiments, the sample is contacted simultaneously or substantially simultaneously with the binding reagent and the detection reagent. In embodiments, the sample is first contacted with the binding reagent, then with the detection reagent. In embodiments, the sample is first contacted with the detection reagent, then with the binding reagent. In embodiments, contacting the sample simultaneously or substantially simultaneously with the binding reagent and the detection reagent does not decrease assay performance, e.g., binding efficiency, assay sensitivity, and/or assay specificity. In embodiments, the biological sample is extracted from the sample collection device into an extraction liquid. It was discovered that by using a low volume of extraction liquid, e.g., 0.3 mL as compared to 3 mL, which is the typical volume used for processing swab samples in a clinical laboratory, the analytical sensitivity for the biological sample is increased, for example, by 10-fold, thereby mitigating loss of assay sensitivity resulting from reduced incubation time.

In embodiments, the assay cartridge is capable of simultaneously performing multiple assays. e.g., a neutralization serology assay or a bridging serology assay as described herein. In embodiments, the assay cartridge simultaneously performs two or more, three or more, four or more, five or more, six or more, seven or more, eight or more, nine or more, or ten or more assays. In an example, the surface of the assay cartridge comprises multiple binding domains, wherein the same or different assays can be performed on each binding domain. Multiple serology assays greatly increase the sensitivity and/or specificity of antibody detection. For example, requiring two independent serology tests to be positive (a “dual-positive requirement approach”) has the potential to greatly increase the specificity (see “FDA Virtual Town Hall Series—Immediately in Effect Guidance on Coronavirus (COVID-19) Diagnostic Tests” dated May 6, 2020). In embodiments, combining the results of multiple serology assays provides a specificity of greater than 99%, greater than 99.5%, greater than 99.9%, or 100%. In embodiments, the assay cartridge is capable of simultaneously performing (1) a classical or bridging serology assay for an antibody that binds the SARS-CoV-2 S protein or variant thereof (e.g., S1, S2, S-NTD, S-RBD, S-ECD); (2) a competitive serology assay that binds the SARS-CoV-2 S protein or variant thereof; and (3) a classical, bridging, or competitive serology assay for an antibody that binds a different SARS-CoV-2 protein (e.g., M, N, or E protein).

In embodiments, the method is performed in the assay cartridge in an automated format. In embodiments, the assay cartridge reader is a free-standing, fully-integrated system for conducting electrochemiluminescence-based immunoassays. In embodiments, the assay cartridge reader is capable of evaluating assay results. In embodiments, the assay cartridge reader performs an assay in about 30 minutes to about 300 minutes, about 30 minutes to about 240 minutes, about 30 minutes to about 180 minutes, about 30 minutes to about 120 minutes, or about 30 minutes to about 60 minutes. An exemplary assay cartridge reader is the MSD® Cartridge Reader instrument. Further exemplary assay cartridges and assay cartridge readers are described, e.g., in U.S. Pat. Nos. 9,921,166; 10,184,884; 9,731,297; 8,343,526; 10,281,678; 10,272,436; US 2018/0074082; and US 2019/0391170.

In embodiments, the method is performed in an assay plate. Assay plates are further described herein. In embodiments, the assay plate comprises one or more wells, chambers, and/or assay regions. In embodiments, the assay plate comprises two or more, six or more, 24 or more, 96 or more, 384 or more, 1536 or more, 6144 or more, or 9600 or more wells, chambers, and/or assay regions. For example, the assay plate can be a multi-well assay plate with a standard well configuration (e.g., a 6-well, 24-well, 96-well, 384-well, 1536-well, 6144-well, or 9600-well plate). In embodiments, each well comprises one or more distinct binding domains. In embodiments, each well comprises at least 2, at least 3, at least 4, at least 5, at least 6, at least 7, at least 8, at least 9, or at least 10 binding domains. In embodiments, each well comprises 4 binding domains. In embodiments, each well comprises 10 binding domains. In embodiments, the assay plate comprises an electrode. In embodiments, the electrode induces luminescence, e.g., electrochemiluminescence, from the materials in the wells, chambers, and/or assay regions. In embodiments, the electrode is a carbon ink electrode. In embodiments, the assay plate comprises a reagent (e.g., any reagent described herein such as binding reagent, detection reagent, anchoring reagent, targeting agent, control reagent, calibration reagent, competitor, or combination thereof) in liquid or dry form in one or more wells, chambers, and/or assay regions of the assay plate. In embodiments, the reagent is immobilized on the surface of the well, chamber, and/or assay region. Exemplary assay plates are disclosed in, e.g., U.S. Pat. Nos. 7,842,246; 8,790,578; and 8,808,627. In embodiments, the assay plate result is read in a plate reader, e.g., the MESO® QUICKPLEX® or MESOK SECTOR® instruments.

In embodiments, the method is performed on a particle. In embodiments, the particle is a bead, e.g., a magnetic bead. In embodiments, the particle comprises a reagent immobilized thereon (e.g., any reagent described herein such as binding reagent, detection reagent, anchoring reagent, targeting agent, control reagent, calibration reagent, competitor, or combination thereof). In embodiments, the particle comprises an electrode. In embodiments, the method comprises collecting the particle on an electrode. For example, magnetic beads can be collected using a magnetic plate. In embodiments, the electrode induces luminescence. e.g., electrochemiluminescence, from the materials on the particle. In embodiments, the electrode is a carbon ink electrode.

Further exemplary devices for performing the methods herein include, but are not limited to, cassettes, measurement cells, dipsticks, reaction vessels, and assay modules described in, e.g., U.S. Pat. Nos. 8,298,934 and 9,878,323.

Assay Methods and Components

The viruses, viral components, and/or biomarkers described herein can be measured using a number of techniques available to a person of ordinary skill in the art, e.g., direct physical measurements (e.g., mass spectrometry) or binding assays (e.g., immunoassays, agglutination assays and immunochromatographic assays). Viruses, viral components, and/or biomarkers identified herein can be measured by any suitable immunoassay method, including but not limited to, ELISA, microsphere-based immunoassay methods, lateral flow test strips, antibody based dot blots or western blots. The method can also comprise measuring a signal that results from a chemical reactions, e.g., a change in optical absorbance, a change in fluorescence, the generation of luminescence, chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques can detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), luminescence, chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques can be used that do not require the use of labels, e.g., techniques based on measuring mass (e.g., surface acoustic wave measurements), refractive index (e.g., surface plasmon resonance measurements), or the inherent luminescence of a virus, viral component, and/or biomarker.

Binding assays for measuring viruses, viral components, and/or biomarkers can use solid phase or homogenous formats. Suitable assay methods include sandwich or competitive binding assays. Examples of sandwich immunoassays are described in U.S. Pat. Nos. 4,168,146 and 4,366,241. Examples of competitive immunoassays include those described in U.S. Pat. Nos. 4,235,601; 4,442,204; and 5,208,535.

Multiple viruses, viral components, and/or biomarkers can be measured using a multiplexed assay format, e.g., multiplexing through the use of binding reagent arrays, multiplexing using spectral discrimination of labels, multiplexing of flow cytometric analysis of binding assays carried out on particles, e.g., using the LUMINEX9® system. Suitable multiplexing methods include array based binding assays using patterned arrays of immobilized binding reagents directed against the viruses, viral components, and/or biomarkers of interest. Various approaches for conducting multiplexed assays have been described (see, e.g., US 2003/0113713; US 2003/0207290; US 2004/0022677; US 2004/0189311; US 2005/0052646; US 2005/0142033; US 2006/0069872; U.S. Pat. Nos. 6,977,722; 7,842,246; 10,189,023; and 10,201,812). One approach to multiplexing binding assays involves the use of patterned arrays of binding reagents, e.g., as described in U.S. Pat. Nos. 5,807,522 and 6,110,426; Delehanty, “Printing functional protein microarrays using piezoelectric capillaries,” Methods Mol Bio 278:135-144 (2004); Lue et al., “Site-specific immobilization of biotinylated proteins for protein microarray analysis,” Methods Mol Biol 278: 85-100 (2004); Lovett, “Toxicogenomics: Toxicologists Brace for Genomics Revolution,” Science 289: 536-537 (2000); Berns, “Cancer: Gene expression in diagnosis,” Nature 403: 491-492 (2000); and Walt, “Molecular Biology: Bead-based Fiber-Optic Arrays,” Science 287: 451-452 (2000). Another approach involves the use of binding reagents coated on beads that can be individually identified and interrogated. See, e.g., WO 99/26067, which describes the use of magnetic particles that vary in size to assay multiple analytes, the particles belonging to different distinct size ranges are used to assay different analytes. The particles are designed to be distinguished and individually interrogated by flow cytometry. Vignali, “Multiplexed Particle-Based Flow Cytometric Assays,” J Immunol Meth 243: 243-255 (2000) describes a multiplex binding assay in which 64 different bead sets of microparticles are employed, each having a uniform and distinct proportion of two. A similar approach involving a set of 15 different beads of differing size and fluorescence has been described as useful for simultaneous typing of multiple pneumococcal serotypes (Park et al., “A Latex Bead-Based Flow Cytometric Immunoassay Capable of Simultaneous Typing of Multiple Pneumococcal Serotypes (Multibead Assay),” Clin Diag Lab Immunol 7: 4869 (2000)). Bishop et al. have described a multiplex sandwich assay for simultaneous quantification of six human cytokines (Bishop et al., “Simultaneous Quantification of Six Human Cytokines in a Single Sample Using Microparticle-based Flow Cytometric Technology,” Clin Chem 45:1693-1694 (1999)).

The methods herein can be conducted in a single assay chamber, such as a single well of an assay plate. The methods herein can also be conducted in an assay chamber of an assay cartridge as described herein. The assay modules, e.g., assay plates or assay cartridges, methods and apparatuses for conducting assay measurements suitable for the present invention, are described, e.g., in U.S. Pat. Nos. 8,343,526; 9,731,297; 9,921,166; U.S. Pat. Nos. 10,184,884; 10,281,678; 10,272,436; US 2004/0022677; US 2004/0189311; US 2005/0052646; US 2005/0142033; US 2018/0074082; and US 2019/0391170.

Binding

Binding reagents that specifically bind to viruses, viral components, and/or biomarkers are described herein. In embodiments, the binding reagent forms a binding complex with its binding partner. In embodiments where the method comprises detecting a coronavirus, the binding complex comprises the binding reagent and the coronavirus component. In embodiments where the method comprises detecting at least one respiratory virus, the binding complex comprises the binding reagent and the respiratory virus component. In embodiments where the method comprises quantifying the amounts of one or more biomarkers capable of binding to a viral antigen (e.g., an antibody biomarker), the binding complex comprises the binding reagent and the antibody biomarker. In embodiments where the method comprises quantifying the amounts of one or more biomarkers, e.g., an inflammatory damage biomarker and/or a tissue damage biomarker, the binding complex comprises the binding reagent and the inflammatory damage biomarker and/or tissue damage biomarker. In embodiments where the biomarker comprises an extracellular vesicle (EV), the binding complex comprises the binding reagent and the EV. In embodiments, the binding reagent is immobilized on a binding domain. In embodiments, the binding complex is formed on the binding domain.

In embodiments where the method is a multiplexed immunoassay method, more than one binding complex is formed, and each binding complex comprises a different binding reagent and its binding partner (e.g., virus, viral component, and/or biomarker described herein). In embodiments, each of the binding reagents are immobilized on separate binding domains. In embodiments, each binding domain comprises a targeting agent capable of binding to a targeting agent complement, wherein the targeting agent complement is connected to a linking agent, and each binding reagent comprises a supplemental linking agent capable of binding to the linking agent. Thus, in embodiments, the binding reagent is immobilized on the binding domain by: (1) binding each binding reagent to the targeting agent complement via the supplemental linking agent and the linking agent; and (2) binding each product of step (1) to a binding domain comprising the targeting agent, wherein (i) each binding domain comprises a different targeting agent, and (ii) each targeting agent selectively binds to one of the targeting agent complements, thereby immobilizing each binding reagent to its associated binding domain.

In embodiments, an optional bridging agent, which is a binding partner of both the linking agent and the supplemental linking agent, bridges the linking agent and supplemental linking agent, such that the binding reagents, each bound to its respective targeting agent complement, are contacted with the binding domains and bind to their respective targeting agents via the bridging agent, the targeting agent complement on each of the binding reagents, and the targeting agent on each of the binding domains.

In embodiments, the targeting agent and targeting agent complement are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin.

In embodiments, the linking agent and supplemental linking agent are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the linking agent and supplemental linking agent are cross-reactive moieties, e.g., thiol and maleimide or iodoacetamide; aldehyde and hydrazide; or azide and alkyne or cycloalkyne. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments that include the optional bridging agent, the bridging agent is streptavidin or avidin, and the linking agents and the supplemental linking agents are each biotin.

In embodiments, each binding domain is an element of an array of binding elements. In embodiments, the binding domains are on a surface. In embodiments, the surface is a plate. In embodiments, the surface is a well in a multi-well plate. In embodiments, the array of binding elements is located within a well of a multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In embodiments, the particle comprises a paramagnetic bead. In embodiments, each binding domain is positioned on one or more particles. In embodiments, the particles are in a particle array. In embodiments, the particles are coded to allow for identification of specific particles and distinguish between each binding domain. In embodiments, the surface is an assay cartridge surface. In embodiments, each binding domain is positioned in a distinct location on the assay cartridge surface.

Detection

In embodiments, the method further comprises detecting the binding complex described herein. In embodiments, the binding complex comprising a binding reagent and its binding partner (e.g., virus, viral component, and/or biomarker described herein) further comprises a detection reagent. In embodiments, the detection reagent specifically binds to the virus, viral component, and/or biomarker described herein.

In embodiments, the method comprises contacting the binding reagent with its binding partner (e.g., virus, viral component, and/or biomarker described herein) and the detection reagent simultaneously or substantially simultaneously to form a binding complex. In embodiments, the method comprises contacting the binding reagent with its binding partner (e.g., virus, viral component, and/or biomarker described herein) and the detection reagent sequentially to form a binding complex. In embodiments, the method comprises contacting the detection reagent with its binding partner (e.g., virus, viral component, and/or biomarker described herein) and the binding reagent sequentially.

In embodiments, the detection reagent is an antibody, antigen, ligand, receptor, oligonucleotide, hapten, epitope, mimotope, or aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof.

In embodiments where the method comprises detecting at least one respiratory virus, the detection reagent comprises an antibody or antigen-binding fragment thereof that specifically binds to the respiratory virus component. In embodiments, the detection reagent comprises a receptor that specifically binds to the respiratory virus component, e.g., ACE2 and/or NRP1. In embodiments where the method comprises detecting a coronavirus, the detection reagent comprises an antibody or antigen-binding fragment thereof that specifically binds to the coronavirus component. In embodiments, the detection reagent comprises a receptor that specifically binds to the coronavirus component, e.g., ACE2 and/or NRP1. In embodiments, the coronavirus is SARS-CoV-2. In embodiments, the coronavirus component is SARS-CoV-2 N protein. In embodiments where the method comprises quantifying the amounts of one or more biomarkers capable of binding to a viral antigen (e.g., an antibody biomarker), the detection reagent comprises an antigen (e.g., a viral protein described herein). In embodiments where the method comprises quantifying the amounts of one or more biomarkers, e.g., an inflammatory damage biomarker and/or a tissue damage biomarker, the detection reagent comprises an antibody or antigen-binding fragment thereof that specifically binds to the inflammatory damage biomarker and/or tissue damage biomarker.

In embodiments, the detection reagent comprises a detectable label. In embodiments, measuring the concentration of the biomarkers in each of the binding complexes comprises measuring the presence and/or amount of the detectable label. In embodiments, the detectable label is measured by light scattering, optical absorbance, fluorescence, luminescence, chemiluminescence, electrochemiluminescence (ECL), bioluminescence, phosphorescence, radioactivity, magnetic field, or combination thereof. In embodiments, the detectable label comprises an electrochemiluminescence label. In embodiments, the detectable label comprises ruthenium. In embodiments, measuring the concentration of the biomarkers comprises measuring the presence and/or amount of the detectable label by electrochemiluminescence. In embodiments, the measuring of the detectable label comprises measuring an electrochemiluminescence signal.

In embodiments, detection reagent comprises a nucleic acid probe. In embodiments, the immunoassay further comprises binding the nucleic acid probe to a template oligonucleotide and extending the nucleic acid probe to form an extended sequence. In embodiments, the extended sequence binds to an anchoring reagent immobilized on the surface comprising the binding reagent. In embodiments, the virus, viral component, and/or biomarker is detected and/or quantified by detecting or quantifying the amount of extended sequence bound to the surface. In embodiments, the surface is contacted with a labeled probe that binds to the extended sequence, wherein the labeled probe comprises a detectable label.

In embodiments, the binding complex comprising the binding reagent and its binding partner (e.g., virus, viral component, and/or biomarker described herein) further comprises a first detection reagent and a second detection reagent. In embodiments, the first detection reagent comprises a first nucleic acid probe, and the second detection reagent comprises a second nucleic acid probe. In embodiments, the immunoassay method further comprises binding the first and second nucleic acid probes to a template oligonucleotide and extending the second nucleic acid probe to form an extended sequence. In embodiments, the extended sequence binds to an anchoring reagent immobilized on the surface comprising the binding reagent. In embodiments, the virus, viral component, and/or biomarker is detected and/or quantified by detecting or quantifying the amount of extended sequence bound to the surface. In embodiments, the surface is contacted with a labeled probe that binds to the extended sequence, wherein the labeled probe comprises a detectable label. Detection methods are further described, e.g., in WO2014/165061; WO2014/160192; WO2015/175856; WO2020/180645; U.S. Pat. No. 9,618,510; U.S. Ser. No. 10/908,157; and U.S. Ser. No. 10/114,015. In embodiments, the method detects the SARS-CoV-2 N protein. In embodiments, the method detects the SARS-CoV-2 S protein. In embodiments, the method detects the SARS-CoV-2 N protein and S protein. In embodiments, the method detects G-CSF, GM-CSF, IFN-α2a, IL-4, IL-6, IL-10, TNF-α, or a combination thereof. In embodiments, the method detects IFN-γ, IL-1β, IL-2, IL-4, IL-6, IL-10. IL-12p70. IL-17A, and TNF-α. In embodiments, the method detects GFAP, Tau, and NF-L. In embodiments, the method detecting GFAP, Tau, and NF-L is an ultrasensitive assay. In embodiments, the method for detecting Tau comprises contacting a sample comprising Tau to a first binding reagent and a second binding reagent on the surface.

In embodiments, the immunoassay comprises:

(a) contacting a biotinylated binding reagent and a biotinylated anchoring reagent with a surface comprising streptavidin or avidin, e.g., for about 30 minutes to about 2 hours at room temperature, or about 6 hours to about 12 hours at 4° C.; and optionally washing the surface to remove unbound binding reagent and/or anchoring reagent;

(b) contacting a sample comprising the analyte of interest (e.g., virus, viral component, and/or biomarker described herein) with the surface, e.g., for about 1 hour to about 2 hours at about 20° C. to about 35° C. (e.g., about 27° C.); and optionally washing the surface to remove unbound analyte;

(c) contacting a detection reagent comprising a nucleic acid probe with the surface, e.g., for about 30 minutes to about 1 hour at about 20° C. to about 35° C. (e.g., about 27° C.), thereby forming a binding complex comprising the binding reagent, the analyte, and the detection reagent; and optionally washing the surface to remove unbound detection reagent;

(d) contacting a template oligonucleotide with the surface and ligating the template oligonucleotide to form a circular template, e.g., for about 10 minutes to about 30 minutes at about 20° C. to about 35° C. (e.g., about 27° C.), thereby hybridizing the nucleic acid probe to the circular template; and optionally washing the surface to remove excess template oligonucleotide;

(e) incubating the surface under conditions sufficient to perform rolling circle amplification, e.g., for about 5 minutes to about 30 minutes, thereby forming an extended sequence that binds to the anchoring reagent;

(f) contacting a labeled probe comprising a detectable label with the surface, e.g., for about 1 hour to about 2 hours at about 20° C. to about 35° C. (e.g., about 27° C.), thereby binding the labeled probe to the extended sequence; and optionally washing the surface to remove excess labeled probe; and

(g) measuring the amount of extended sequence by quantifying the amount of detectable label, thereby detecting and/or measuring the amount of analyte (e.g., virus, viral component, and/or biomarker described herein) in the sample. In embodiments, the virus is a coronavirus. In embodiments, the virus is SARS-CoV-2. In embodiments, the SARS-CoV-2 component is SARS-CoV-2 N protein.

In embodiments, steps (b) and (c) are performed simultaneously or substantially simultaneously. In embodiments, performing steps (b) and (c) simultaneously or substantially simultaneously does not substantially change the sensitivity and/or specificity of the immunoassay. In embodiments, performing steps (b) and (c) simultaneously or substantially simultaneously does not substantially change the ability of the binding reagent and/or the detection reagent to bind the analyte of interest (e.g., virus, viral component, and/or biomarker described herein). In embodiments, the virus is a coronavirus. In embodiments, the virus is SARS-CoV-2. In embodiments, the SARS-CoV-2 component is SARS-CoV-2 N protein.

In embodiments, step (e) is performed for less than 30 minutes, less than 25 minutes, less than 20 minutes, less than 15 minutes, less than 10 minutes, or about 5 minutes. In embodiments, the amount of rolling circle amplification product generated after 5 minutes of amplification is sufficient for detection of the analyte (e.g., virus, viral component, and/or biomarker described herein). In embodiments, the virus is a coronavirus. In embodiments, the virus is SARS-CoV-2. In embodiments, the SARS-CoV-2 component comprises an N protein, an S protein, or both. In embodiments, the SARS-CoV-2 component is SARS-CoV-2 N protein.

In embodiments, the invention provides a method for detecting SARS-CoV-2 in a biological sample, comprising: (a) contacting the biological sample with (i) a surface comprising a binding reagent, wherein the binding reagent comprises biotin and the surface comprises avidin or streptavidin, wherein the binding reagent specifically binds a SARS-CoV-2 component; and (ii) a detection reagent that specifically binds the SARS-CoV-2 component, wherein the detection reagent comprises a detectable label, thereby forming a binding complex on the surface comprising the binding reagent, the SARS-CoV-2 component, and the detection reagent; (b) detecting the binding complex, thereby detecting SARS-CoV-2 in the biological sample. In embodiments, the SARS-CoV-2 component comprises an N protein, an S protein, or both. In embodiments, the SARS-CoV-2 component is SARS-CoV-2 N protein.

In embodiments, the immunoassay comprises:

(a) contacting a coating solution comprising a binding reagent bound to a linking agent with a surface comprising one or more binding domains, wherein each binding domain comprises a targeting agent, and wherein the binding reagent specifically binds a SARS-CoV-2 component;

(b) contacting each binding domain with: (i) a sample comprising the SARS-CoV-2 component, (ii) a calibration reagent, or (iii) a control reagent;

(c) contacting each binding domain with a detection reagent that specifically binds the SARS-CoV-2 component, wherein the detection reagent comprises a detectable label;

(d) measuring the amount of detectable label on the surface, thereby detecting and/or measuring the amount of the SARS-CoV-2 component. In embodiments, the SARS-CoV-2 component is SARS-CoV-2 N protein. In embodiments, the SARS-CoV-2 component is SARS-CoV-2 S protein.

In embodiments, the immunoassay comprises:

(a) contacting a biotinylated binding reagent with a surface comprising one or more binding domains, wherein each binding domain comprises avidin or streptavidin;

(b) contacting each binding domain with: (i) a sample comprising the SARS-CoV-2 component, (ii) a calibration reagent, or (iii) a control reagent;

(c) contacting each binding domain with a detection reagent that specifically binds the SARS-CoV-2 component, wherein the detection reagent comprises a detectable label;

(d) measuring the amount of detectable label on the surface, thereby detecting and/or measuring the amount of the SARS-CoV-2 component. In embodiments, the SARS-CoV-2 component is SARS-CoV-2 N protein or SARS-CoV-2 S protein. In embodiments, the immunoassay is a multiplexed immunoassay for detecting both the SARS-CoV-2 N protein and the SARS-CoV-2 S protein.

In embodiments, the surface is a multi-well plate. In embodiments, the method further comprises a wash step prior to one or more of the method steps. In embodiments, the wash step comprises washing the assay plate at least once, at least twice, at least three times, at least four times, or at least five times with a wash buffer. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, prior to step (a), a blocking solution is added to the plate to reduce non-specific binding to the surface. In embodiments, about 50 μL to about 250 μL, about 100 μL to about 200 μL, or about 150 μL of blocking solution is added per well of the plate. In embodiments, the plate is incubated at about 15° C. to about 30° C., about 18° C. to about 28° C., about 20° C. to about 26° C., or about 22° C. to about 24° C. In embodiments, the plate is incubated while shaken at about at about 500 rpm to about 2000 rpm, about 600 rpm to about 1500 rpm, or about 700 rpm to about 1000 rpm. In embodiments, the method comprises incubating the blocking solution on the plate for about 10 minutes to about 2 hours, about 20 minutes to about 90 minutes, or about 30 minutes to about 60 minutes. In embodiments, the plate is incubated at about room temperature (e.g., about 22° C. to about 28° C.) while shaken at about 700 rpm for about 30 minutes to about 1 hour.

In embodiments comprising a coating solution, the method further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL, or about 400 μL to about 600 μL of the coating solution. In embodiments, step (a) comprises incubating the linking agent and the binding reagent at about 15° C. to about 30° C., about 18° C. to about 28° C., about 20° C. to about 26° C., or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the method further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C. about 18° C. to about 28° C., about 20° C. to about 26° C. or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, step (a) comprises adding about 10 μL to about 200 μL, about 5 μL to about 100 μL, about 10 μL to about 90 μL, about 15 μL to about 80 μL, about 20 μL to about 70 μL, about 30 μL to about 60 μL, or about 50 μL of the coating solution or a solution containing the biotinylated binding reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (b) comprises adding about 5 μL to about 50 μL, about 10 μL to about 40 μL, about 20 μL to about 30 μL, about 15 μL, about 25 μL, or about 50 μL of the sample, calibration reagent, or control reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (c) comprises adding about 5 μL to about 50 μL, about 10 μL to about 40 μL, about 10 μL to about 20 μL, about 20 μL to about 30 μL, about 15 μL, about 25 μL, or about 50 μL of a solution comprising the detection reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

In embodiments, the surface comprising the binding domains described herein comprises an electrode. In embodiments, the electrode is a carbon ink electrode. In embodiments, the measuring of the detectable label comprises applying a potential to the electrode and measuring electrochemiluminescence. In embodiments, applying a potential to the electrode generates an electrochemiluminescence signal. In embodiments, the strength of the electrochemiluminescence signal is based on the amount of detected virus, viral component, and/or biomarker in the binding complex.

In embodiments, the immunoassay described herein further comprises measuring the concentration of one or more calibration reagents. In embodiments, a calibration reagent comprises a known concentration of a virus, viral component, or biomarker described herein. In embodiments, the calibration reagent comprises a mixture of known concentrations of multiple viruses, viral components, or biomarkers. In embodiments, the immunoassay further comprises measuring the concentration of multiple calibration reagents comprising a range of concentrations for one or more viruses, viral components, or biomarkers. In embodiments, the multiple calibration reagents comprise concentrations of one or more viruses, viral components, or biomarkers near the upper and lower limits of quantitation for the assay. In embodiments, the multiple concentrations of the calibration reagent spans the entire dynamic range of the immunoassay. In embodiments, the calibration reagent is a negative control, i.e., containing no viruses, viral components, or biomarkers.

In embodiments, the immunoassay described herein for detection of a virus or viral component has a detection limit of less than 150 TCID50/mL, less than 100 TCID50/mL, less than 90 TCID50/mL, less than 80 TCID50/mL, less than 70 TCID50/mL, less than 60 TCID50/mL, less than 50 TCID50/mL, less than 40 TCID50/mL, less than 30 TCID50/mL, less than 20 TCID50/mL, less than 10 TCID50/mL, less than 5 TCID50/mL, less than 4 TCID50/mL, less than 3 TCID50/mL, less than 2 TCID50/mL, less than 1 TCID50/mL, less than 0.5 TCID50/mL, or less than 0.1 TCID50/mL.

In embodiments, the immunoassay described herein for the detection of a viral protein, e.g., the SARS-CoV-2 N protein, in a biological sample, has substantially the same or higher sensitivity for determining viral load as an assay, e.g., a PCR-based assay, that detects a viral nucleic acid, e.g., SARS-CoV-2 RNA, in the biological sample. In embodiments, the immunoassay for the detection of a viral protein, e.g., the SARS-CoV-2 N protein, in a biological sample, has substantially the same or higher specificity for determining viral load as an assay, e.g., a PCR-based assay, that detects a viral nucleic acid, e.g., SARS-CoV-2 RNA, in the biological sample. In embodiments, the biological sample for the immunoassay described herein is a nasopharyngeal sample. In embodiments, the biological sample for the immunoassay described herein is a saliva sample. In embodiments, the immunoassay described herein for detecting a viral protein, e.g., the SARS-CoV-2 N protein, in a saliva sample has substantially the same or higher sensitivity as a PCR-based assay for detecting a viral nucleic acid, e.g., SARS-CoV-2 RNA, in a nasopharyngeal sample. See, e.g., Ren et al., medRxiv pre-print doi: 10.1101/2021.02.17.21251863 (19 Feb. 2021). In embodiments, the immunoassay described herein for detection of a viral protein is capable of detecting viral infection at an earlier stage as compared with a PCR-based assay, e.g., an RT-PCR assay. In embodiments, the amount of viral protein, e.g., SARS-CoV-2 N protein, as measured by an immunoassay described herein is directly correlated with the amount of viral nucleic acid, e.g., as measured by a PCR-based assay such as an RT-PCR assay. In embodiments, the virus is a coronavirus. In embodiments, the virus is SARS-CoV-2. In embodiments, the viral protein is SARS-CoV-2 N protein. Analysis of immunoassays for detection of viral components and PCR-based assays is further described in, e.g., Pollock et al., “Correlation of SARS-CoV-2 nucleocapsid antigen and RNA concentrations in nasopharyngeal samples from children and adults using an ultrasensitive and quantitative antigen assay,” J Clin Microbiol (2021), doi:10.1128/JCM.03077-20.

Assays for Extracellular Vesicles and Intact Viruses

In embodiments where the biomarker comprises an extracellular vesicle (EV), multiple binding and/or detection reagents bind to a surface marker of the EV. In embodiments, the binding reagent comprises an antibody or antigen-binding fragment thereof that specifically binds to a viral protein on the surface of the EV. In embodiments, the binding reagent comprises an antibody or antigen-binding fragment thereof that specifically binds to a viral protein on the surface of the EV. In embodiments, the binding reagent comprises an antibody or antigen-binding fragment thereof that specifically binds to an inflammatory damage biomarker and/or a tissue damage biomarker on the surface of the EV. In embodiments, the binding reagent comprises an antibody or antigen-binding fragment thereof that specifically binds to a tissue-specific marker on the surface of the EV. A “tissue-specific marker” is a biomarker that is specifically expressed in one type of host tissue, e.g., brain, kidney, intestines, or respiratory tract. In embodiments, the binding reagent comprises an antibody or antigen-binding fragment thereof that specifically binds to a common EV surface marker. In embodiments, the common EV surface marker comprises CD81, CD9, CD63, or combination thereof.

In embodiments, the detection reagent comprises an antibody or antigen-binding fragment thereof that specifically binds to a viral protein on the surface of the EV. In embodiments, the detection reagent comprises an antibody or antigen-binding fragment thereof that specifically binds to a viral protein on the surface of the EV. In embodiments, the viral protein is an S protein or subunit or fragment thereof from SARS-CoV-2, an M protein from SARS-CoV-2, an E protein from SARS-CoV-2, or a combination thereof. In embodiments, the detection reagent comprises an antibody or antigen-binding fragment thereof that specifically binds to an inflammatory damage biomarker and/or a tissue damage biomarker on the surface of the EV. In embodiments, the detection reagent comprises an antibody or antigen-binding fragment thereof that specifically binds to a tissue-specific marker on the surface of the EV. In embodiments, the detection reagent comprises an antibody or antigen-binding fragment thereof that specifically binds to a common EV surface marker. In embodiments, the common EV surface marker comprises CD81, CD9, CD63, or combination thereof.

In embodiments, the binding reagent and the detection reagent bind to different surface markers on the EV. In embodiments, one or more binding reagents and one or more detection reagents each binds to a different common EV surface marker. e.g., CD81, CD9, and CD63. In embodiments, one of the binding or detection reagent binds to a host protein (e.g., a tissue-specific marker or an inflammatory damage and/or tissue damage biomarker) on the surface of the EV, and the other of the binding or detection reagent binds to a viral protein on the surface of the EV. In embodiments, the binding reagent binds a viral protein (e.g., an S protein or subunit or fragment thereof from SARS-CoV-2, an M protein from SARS-CoV-2, an E protein from SARS-CoV-2, or a combination thereof), and one or more detection reagents binds CD81, CD9, CD63, or a combination thereof. In embodiments, the EV is contacted with two, three, four, or more than four binding and/or detection reagents, each binding to a different surface marker on the EV. In embodiments, the EV is contacted with one binding reagent and one detection reagent. In embodiments, the EV is contacted with one binding reagent and two detection reagents. In embodiments, the EV is contacted with one binding reagent and three detection reagents. In embodiments, the EV is contacted with one or more binding reagents and one, two, three, or more than three detection reagents.

Detection of EVs from infected cells can be useful in identifying reservoirs of infection. EV populations in a biological sample can also be analyzed to determine the mechanism of infection, disease prognosis, and adaptive immunity. In embodiments, the EV is from a cell infected by a virus described herein, e.g., a coronavirus such as SARS-CoV-2. In embodiments, the EV is detected as an intact EV, e.g., without disrupting the EV membrane. In embodiments, a binding complex on a surface is formed comprising the binding reagent and the EV, and the EV is then lysed and contacted with a detection reagent that binds to a component inside the EV (also referred to herein as an “EV cargo”), thereby detecting the EV cargo. In embodiments, the EV cargo comprises a host biomarker, a viral component, a nucleic acid, a lipid, a small molecule, or combination thereof.

In embodiments, the method for detecting an EV in a biological sample comprises: a) contacting the biological sample with (i) a binding reagent immobilized on a surface that binds to a first surface marker on the EV; (ii) a first detection reagent that binds to a second surface marker on the EV, wherein the first detection reagent comprises a first nucleic acid probe; and (iii) a second detection reagent that binds to a third surface marker on the EV, wherein the second detection reagent comprises a second nucleic acid probe, thereby forming a binding complex on the surface comprising the EV, the binding reagent, and the first and second detection reagents; b) using an extension process that requires the first and second nucleic acid probes to be in proximity, extending the second nucleic acid probe to form an extended oligonucleotide; binding the extended oligonucleotide to the surface; and measuring the amount of extended oligonucleotide bound to the surface, thereby detecting the EV in the biological sample. In embodiments, the method requires the binding of three different EV surface markers by the binding and detection reagents, thereby increasing specificity of the method.

An embodiment of the method for detecting an EV is illustrated in FIG. 15C. A surface comprising a binding reagent for a viral antigen captures an intact EV comprising a viral antigen on the EV surface. A pair of detection reagents binds to two host proteins in proximity on the EV surface. The detection reagents can include nucleic acid probes, which can be extended to form an extended oligonucleotide, and the extended oligonucleotide is bound to the surface and detected as described herein. In embodiments, the EV is detected only if all three markers (viral antigen bound by binding reagent and two host proteins bound by detection reagents) are present on the EV.

In embodiments, a calibration reagent for the EV detection assay comprises an engineered EV that comprises one or more viral antigens (e.g., from a coronavirus such as SARS-CoV-2) and/or host biomarkers (e.g., a tissue-specific marker or an inflammatory and/or tissue-damage biomarker) on its surface. In embodiments, the engineered EV comprises the S protein, E protein, M protein, or combination thereof, on its surface. In embodiments, the engineered EV comprises the N protein encapsulated therein, e.g., as an EV cargo. In embodiments, the engineered EV comprising the N protein is a calibration reagent for an assay that for detecting an EV cargo, as described herein.

In embodiments, the invention further provides a method for assessing the temporal profile of viral component production and/or turnover, comprising quantifying the amount of EVs comprising a viral antigen, as described herein, and correlating the amount of EVs with the viral RNA detected at different time points. In embodiments, the virus is a coronavirus. In embodiments, the virus is SARS-CoV-2.

In embodiments, the method comprises detecting an intact virus, e.g., a respiratory virus such as coronavirus as described herein. In embodiments, detection of an intact virus improves the accuracy and specificity of an infection diagnosis compared with detection of an individual viral component. For example, individual viral components may be present in a biological sample even after an infection is being cleared or has cleared from a subject. Thus, detection of an intact virus is more closely associated with an active infection. In embodiments, the virus is a coronavirus. In embodiments, the virus is SARS-CoV-2. Detection and analysis of intact viruses and other surface-marker displaying agents, e.g., EVs, are further described, e.g., in WO 2019/222708 and WO 2020/086751.

In embodiments, the binding reagent binds to a viral antigen on the surface of the virus to form a binding complex, and the detection reagent binds to a different viral antigen on the surface of the virus to detect an intact virus. In embodiments, the intact virus is contacted with two, three, four, or more than four binding and/or detection reagents, each binding to a different viral antigen on the surface of the virus. In embodiments, the intact virus is contacted with one binding reagent and one detection reagent. In embodiments, the intact virus is contacted with one binding reagent and two detection reagents. In embodiments, the intact virus is contacted with one binding reagent and three detection reagents. In embodiments, the intact virus is contacted with one or more binding reagents and one, two, three, or more than three detection reagents. In embodiments, the viral antigen on the surface of the virus is a structural protein as described herein. In embodiments, an intact virus is contacted with one or more binding reagents and one or more detection reagents that bind to an S protein or subunit thereof such as the S1 subunit, S2 subunit, or S-RBD, an E protein, and an M protein. In embodiments, an intact virus is contacted with a binding reagent and a detection reagent, wherein the binding and detection reagent each binds to a different protein selected from a S protein and an E protein. In embodiments, an intact virus is contacted with a binding reagent and a detection reagent, wherein the binding and detection reagent each binds to a different protein selected from a S protein and an M protein. In embodiments, an intact virus is contacted with a binding reagent and a detection reagent, wherein the binding and detection reagent each binds to a different protein selected from an E protein and an M protein. In embodiments, an intact virus is contacted with a binding reagent, a first detection reagent, and a second detection reagent, wherein each of the binding and detection reagents binds to a different protein selected from a S protein, an E protein, and a M protein. In embodiments, an intact virus is contacted with a binding reagent, a first detection reagent, and a second detection reagent, wherein each of the binding and detection reagents bind to a different protein selected from an S1 subunit, an S2 subunit, and an E protein. In embodiments, an intact virus is contacted with a binding reagent, a first detection reagent, and a second detection reagent, wherein each of the binding and detection reagents bind to a different protein selected from an S1 subunit, an S2 subunit, and an M protein. In embodiments, one of the binding or detection reagent binds to a host protein (e.g., a tissue-specific marker or an inflammatory damage and/or tissue damage biomarker as described herein) on the intact virus, and the other of the binding or detection reagent binds to a viral antigen on the surface of the virus. In embodiments, the virus is a coronavirus. In embodiments, the virus is SARS-CoV-2.

In embodiments, the method for detecting an intact virus in a biological sample comprises: a) contacting the biological sample with (i) a binding reagent immobilized on a surface that binds to a first viral antigen on the viral surface; (ii) a first detection reagent that binds to a second viral antigen on the viral surface, wherein the first detection reagent comprises a first nucleic acid probe; and (iii) a second detection reagent that binds to a third viral antigen on the viral surface, wherein the second detection reagent comprises a second nucleic acid probe, thereby forming a binding complex on the surface comprising the virus, the binding reagent, and the first and second detection reagents; b) using an extension process that requires the first and second nucleic acid probes to be in proximity, extending the second nucleic acid probe to form an extended oligonucleotide; binding the extended oligonucleotide to the surface; and measuring the amount of extended oligonucleotide bound to the surface, thereby detecting the intact virus in the biological sample. In embodiments, the method requires the binding of three different viral antigens by the binding and detection reagents, thereby increasing specificity of the method. In embodiments, the virus is a coronavirus. In embodiments, the virus is SARS-CoV-2.

An embodiment of the method for detecting an intact virus is illustrated in FIG. 15B. A surface comprising a binding reagent for a first viral antigen captures a virus by binding to the first viral antigen on the viral surface. A pair of detection reagents binds to second and third viral antigens in proximity on the viral surface. The detection reagents can include nucleic acid probes, which can be extended to form an extended oligonucleotide, and the extended oligonucleotide is bound to the surface and detected as described herein. In embodiments, the virus is detected only if all three viral antigens (bound by the binding reagent and detection reagents) are present on the virus. In embodiments, the virus is a coronavirus. In embodiments, the virus is SARS-CoV-2.

In embodiments, the method comprises detecting an inactivated respiratory virus as described herein, e.g., a coronavirus such as SARS-CoV-2. In embodiments, the method comprises detecting a virus-like particle (VLP) of a respiratory virus as described herein, e.g., a coronavirus such as SARS-CoV-2. In embodiments, an inactivated virus and/or a VLP has the same or substantially similar structure or structural proteins as a wild type virus but is incapable of infecting (e.g., interacting with the host cell receptor or binding partner) and/or replication. In embodiments, the inactivated virus and/or VLP is a calibration reagent for the intact virus assay. In embodiments, the inactivated virus and/or VLP comprises substantially the same viral antigens on its surface as wild-type virus, e.g., a coronavirus such as SARS-CoV-2. In embodiments, the inactivated virus and/or VLP comprises the S protein, E protein, M protein, or combination thereof, on its surface. In embodiments, the inactivated virus and/or VLP comprises the N protein encapsulated therein. In embodiments, the inactivated virus and/or VLP comprising the N protein is a calibration reagent for an assay for detecting an internal component of a virus, e.g., a component that is not present on the viral surface. In embodiments, the virus is SARS-CoV-2.

In embodiments, the invention further provides a method for assessing the temporal profile of viral component production and/or turnover, comprising quantifying the amount of a virus, as described herein, and correlating the amount of virus with the viral RNA detected at different time points. In embodiments, the virus is a coronavirus. In embodiments, the virus is SARS-CoV-2.

In embodiments, an intact virus, e.g., a coronavirus such as SARS-CoV-2, and an EV from a cell infected by the virus are distinguishable from each other by their surface marker(s). Without being bound by a particular theory, it is believed that for most coronaviruses (such as, e.g., SARS-CoV-2), the membrane (M) and spike (S) proteins constitute the majority of the protein that is incorporated into the viral envelope, whereas only a few molecules of the E protein (which is indispensable for viral assembly) are present in the viral envelope. See, e.g., Venkatagopalan et al., Virology 478:75-85 (2015). Coronaviruses have been shown to bud into the endoplasmic reticulum-Golgi intermediate compartment (ERGIC) of its host cell, where the virus acquires its membrane envelope. Once in the lumen of the ERGIC, infectious virions make their way through the host secretory pathway to, ultimately, be released from the infected cell. Accordingly, the E protein of the coronavirus is localized mainly to the ER and Golgi-complex, where it participates in the assembly, budding, and intracellular trafficking of infectious virions. See, e.g., Schoeman et al., Virology Journal 16:69 (2019). However, EVs do not typically arise from the ERGIC. In general, EVs bud directly from the plasma membrane and are thus likely devoid of the E protein.

Subcellular localization of the M protein varies across coronavirus species. For example, the M protein of beta coronaviruses (such as HCoV-HKU1, MERS-CoV, and SARS-CoV) is expressed in the endoplasmic reticulum (ER) of the host cell and largely retained in the trans-Golgi network, and are thus also unlikely to be incorporated into EVs. The S protein has been observed to be expressed in the plasma membrane for SARS-CoV, and thus, may be incorporated into EVs.

Competitive Assays

In embodiments, the methods provided herein are in a competitive assay format. In general terms, a competitive assay, e.g., a competitive immunoassay or a competitive inhibition assay, an analyte (e.g., a biomarker described herein) and a competitor compete for binding to a binding reagent (e.g., a viral antigen described herein). In such assays, the analyte is typically indirectly measured by directly measuring the competitor. As used herein, “competitor” refers to a compound capable of binding to the same binding reagent as an analyte, such that the binding reagent can only bind either the analyte or the competitor, but not both. In embodiments, competitive assays are used to detect and measure analytes that are not capable of binding more than one binding reagents, e.g., small molecule analytes or analytes that do not have more than one distinct binding sites. Examples of competitive immunoassays include those described in U.S. Pat. Nos. 4,235,601; 4,442,204; and 5,028,535.

Assays for Antibody Biomarkers

In embodiments where the biomarker detected is an antibody, e.g., an antibody capable of binding to a viral antigen such as S, S1, S2, S-NTD, S-ECD, S-RBD, M, E. or N, the binding reagent is an antigen that is bound by the antibody biomarker. In embodiments, antibody biomarkers are detected using a bridging serology assay. In a bridging serology assay, the binding complex further comprises a detection reagent described herein, and both the binding reagent and the detection reagent are an antigen that that is bound by the antibody biomarker. Since antibodies are typically bivalent, the antibody biomarker can bind both the binding reagent antigen and the detection reagent antigen.

In embodiments, antibody biomarkers are detected using a regular bridging serology assay. In a regular bridging serology assay, the antibody biomarker, binding reagent antigen, and detection reagent antigen are incubated together to form a complex where the antibody biomarker bivalently binds both the binding reagent antigen and the detection reagent antigen, e.g., a bridged complex. The incubation can be performed in any appropriate container, for example, in the well of a polypropylene plate, or in a chamber of an assay cartridge. In embodiments, the binding reagent antigen is conjugated to a biotin, and the bridged complex solution can be transferred to contact a surface comprising streptavidin, e.g., a streptavidin plate. In this embodiment, the biotin conjugated to the binding reagent antigen binds to the streptavidin plate, causing the entire bridged complex to be immobilized on the streptavidin plate.

In embodiments, antibody biomarkers are detected using a stepwise bridging serology assay. In a first step of a stepwise bridging serology assay, the binding reagent antigen is first immobilized on a surface. In embodiments where the binding reagent antigen is conjugated to biotin, the binding reagent antigen can be immobilized on a streptavidin plate. In a second step, after the binding reagent antigen is immobilized on the surface, a solution containing the antibody biomarker is contacted with the surface, allowing the first bivalent position on the antibody biomarker to bind the binding reagent antibody. In a third step, the detection reagent antigen is then contacted with the surface, allowing the second bivalent position on the antibody to bind the detection reagent antibody. In this stepwise method, the bridging complex is formed stepwise on the surface, rather than forming the entire bridging complex before immobilization, as is done in the regular bridging assay described above. In the stepwise bridging assay, the surface may optionally be rinsed or washed between any of the steps.

In either of the regular bridging serology assay or stepwise bridging serology assay, a method may be used where the detectable label is not directly conjugated to the detection reagent antigen but is instead attached to the detection antigen reagent using a binding complex such as streptavidin/biotin or other binding pair. The advantage of using this method is that it is not necessary to prepare separately conjugated binding reagent antigen and detection reagent antigen. In a non-limiting example of this method, a biotin conjugated antigen is prepared. Some of this biotin conjugated antigen is then incubated with a detectable label conjugated with streptavidin. The binding of biotin to streptavidin causes the detectable label to become attached to the biotin conjugated antigen, creating a detection reagent antigen comprising a detectable label as follows:

Antigen-biotin-streptavidin-detectable label

In embodiments, additional free biotin is added to the antigen-detectable label reagent to fully occupy the streptavidin binding sites and prevent other biotin conjugates from binding to the antigen-detectable label reagent. An additional amount of the biotin conjugated antigen, which is not attached to a detectable label, is then used as the binding reagent antigen. Binding reagent antigen and detection reagent antigen prepared in this way may be used in any of the assay methods described herein.

In embodiments, the antibody biomarker is detected using a classical serology assay. In embodiments of a classical serology assay, the binding reagent is an antigen that is bound by the antibody biomarker. After the antibody biomarker is bound by the binding reagent antigen, the binding complex is detected using a detection reagent antibody that binds the antibody biomarker. In embodiments, the detection reagent antibody is an anti-human antibody that binds human antibody biomarkers. In embodiments, the detection reagent antibody is an anti-human IgG, an anti-human IgM or an anti-human IgA isotype antibody. In embodiments, the detection reagent antibody is an anti-mouse antibody that binds mouse antibody biomarkers, or an anti-rat antibody that binds rat antibody biomarkers, or an anti-ferret antibody that binds ferret antibody biomarkers, or an anti-minx antibody that binds minx antibody biomarkers, or an anti-bat antibody that binds bat antibody biomarkers. In embodiments, the detection reagent antibody is an anti-mouse IgG. IgM, or IgA antibody, an anti-rat IgG. IgM, or IgA antibody, an anti-ferret IgG, IgM, or IgA antibody, an anti-minx IgG, IgM, or IgA antibody, or an anti-bat IgG. IgM, or IgA antibody.

In embodiments, the antibody biomarker is detected using a competitive serology assay (also termed a neutralization serology assay). Competitive immunoassays are described herein. In embodiments of a competitive serology assay, the binding reagent is an antigen that is bound by the antibody biomarker and by a competitor. In embodiments, the competitor is a substance that binds a specific region of the viral antigen. In embodiments, the competitor is a recombinant antibody or antigen-binding fragment thereof that binds specifically to an epitope of the viral antigen, e.g., a neutralizing epitope. In embodiments, the competitor is a monoclonal antibody against an epitope of the viral antigen, e.g., a neutralizing epitope. In embodiments, the competitor comprises a detectable label described herein. For example, the biomarker can be an antibody that binds specifically to a coronavirus spike protein, and the competitor can be the ACE2 receptor, NRP1 receptor, or CD147, i.e., natural interaction partners of the spike protein. In embodiments, the competitor is the ACE2 receptor. In embodiments, the receptor is the NRP1 receptor. In embodiments, the competitor is CD147. In embodiments, the competitor comprises a sialic acid. In embodiments, the binding reagent is a substance that binds a viral antigen (e.g., ACE2, NRP1, or CD147), and the competitor is the viral antigen (e.g., spike protein or a variant thereof described herein, such as, e.g., S1, S2, S-NTD, S-ECD, or S-RBD). In embodiments, the coronavirus is SARS-CoV-2. In embodiments, a competitive serology assay as described herein is used to assess a potential protective serological response, e.g., the ability of the immune response to block binding of a viral antigen to its host cell receptor such as ACE2, NRP1, or CD147.

In embodiments, the antibody biomarker serology assay (either bridging, classical, or competitive) described herein comprises measuring the concentration of one or more calibration reagents. In embodiments, the calibration reagent is a positive control. In embodiments, the positive control comprises an antigen for which an antibody is known or expected to be present in the biological sample. In embodiments, the positive control comprises an antigen from a prevalent influenza strain, to which most subjects are expected to have antibodies. In embodiments, the positive control is an antigen from the H1 Michigan influenza virus. In embodiments, the positive control is immobilized in a binding domain of a surface that further comprises one or more viral antigens immobilized thereon in one or more additional binding domains, as described herein. In embodiments, antibody biomarker serology assay further comprises measuring the total levels of a particular antibody, e.g., total IgG, IgA, or IgM.

In embodiments, the calibration reagent is a negative control. In embodiments, the negative control comprises an antigen for which no antibodies are expected to be present in the biological sample. In embodiments, the negative control comprises a substance obtained from a non-human subject, and the biological sample is obtained from a human subject. In embodiments, the negative control comprises bovine serum albumin (BSA). In embodiments, the negative control, e.g., BSA, is immobilized in a binding domain of a surface that further comprises one or more viral antigens immobilized thereon in one or more additional binding domains, as described herein.

In embodiments, the calibration reagent comprises a combination of biological samples from subjects known to be infected or exposed to a virus described herein. In embodiments, the calibration reagent comprises a pooled sample of serum and/or plasma from subjects known to be infected or exposed to a virus described herein. In embodiments, the calibration reagent is the same biological material as the sample to be assayed. For example, if the biological sample for the antibody biomarker serology assay is a serum sample, then the calibration reagent is a pooled serum sample. Similarly, if the biological sample for the antibody biomarker serology assay is a plasma sample, then the calibration reagent is a pooled plasma sample. In embodiments, the pooled sample comprises a known amount of IgG, IgA, and/or IgM that specifically bind to one or more viral antigens of interest. Methods of measuring IgG, IgA, and/or IgM concentration in a serum or plasma sample is known in the art, e.g., as described in Quataert et al., Clinical and Diagnostic Laboratory Immunology 2(5):590-597 (1995). In embodiments, the antibody biomarker serology assay comprises measuring the concentration of viral antigen-specific IgG, IgA, and/or IgM in multiple pooled samples to provide a calibration curve. In embodiments, the antibody biomarker serology assay comprises measuring the concentration of viral antigen-specific IgG, IgA, and/or IgM in multiple pooled samples, wherein the multiple pooled samples correspond to high, medium, and low levels of viral antigen-specific IgG, IgA, and/or IgM (referred to herein as “high pooled sample,” “medium pooled sample,” and “low pooled sample.” respectively). In embodiments, the pooled sample comprises serum and/or plasma from subjects known to never have been exposed to a virus described herein, i.e., a negative pooled sample. Pooled samples as calibration reagents allow baseline immune response thresholds to be defined and provides a better understanding of the levels of antibody response to a viral infection. In embodiments, the virus is a coronavirus. In embodiments, the virus is SARS-CoV-2.

In embodiments, the biological sample for the antibody biomarker serology assay is a saliva sample, and the calibration reagent comprises a calibration saliva sample. In embodiments, the calibration saliva sample contains a known amount of viral antigen-specific IgG, IgA, and/or IgM. In embodiments, the calibration saliva sample comprises serum from a subject known to be infected or exposed to a virus described herein. In embodiments, the calibration saliva sample comprises about 0.1% to about 1% of high pooled serum sample described herein. In embodiments, the calibration saliva sample comprises about 0.1%, about 0.2%, about 0.3%, about 0.4%, or about 0.5% of high pooled serum sample described herein. In embodiments, the calibration saliva sample comprises levels of viral antigen-specific IgG, IgA, and/or IgM equivalent to a 1:500 dilution of the high pooled serum sample as described herein. In embodiments, the calibration saliva sample is obtained from a subject known to never have been exposed to a virus described herein, i.e., a negative saliva sample. In embodiments, the calibration saliva sample provides a consistent threshold for comparing viral antigen-specific IgG, IgA, and/or IgM levels in saliva samples. In embodiments, the virus is a coronavirus. In embodiments, the virus is SARS-CoV-2.

In embodiments, the calibration reagents, e.g., the pooled sample and/or the calibration saliva sample described herein, is subjected to an antibody biomarker serology assay, e.g., the classical, bridging, and/or competitive serology assays described herein. In embodiments, the assay comprises measuring the total amount of IgG, IgA, and/or IgM in a dilution series of the calibration reagent. In embodiments, the assay further comprises generating a standard curve based on the measured amounts of IgG, IgA, and/or IgM in the calibration reagent dilution series. In embodiments, the assay comprises determining the amount of IgG, IgA, and/or IgM in a biological sample based on the standard curve. In embodiments, the IgG. IgA, and/or IgM is from a human, a mouse, a rat, a ferret, a minx, a bat, or a combination thereof.

An exemplary multiplexed serology assay detecting human IgG and/or IgM against SARS-CoV-2 antigens, as described in embodiments herein, comprises:

1A. Preparation of assay plate. In embodiments, the assay plate is a 384-well assay plate. In embodiments, the assay plate is a 96-well assay plate. In embodiments, each well comprises four distinct binding domains. In embodiments, the first binding domain comprises an immobilized SARS-CoV-2 S protein, the second binding domain comprises an immobilized SARS-CoV-2 N protein, and the third binding domain comprises an immobilized SARS-CoV-2 S-RBD. In embodiments, the fourth binding domain comprises a control protein that does not bind to human IgG or IgM. In embodiments, the fourth binding domain comprises immobilized BSA. An embodiment of a well in a 384-well assay plate, comprising four binding domains (“spots”), is shown in FIG. 39A. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized SARS-CoV-2 S-RBD, and Spot B2 of FIG. 39A comprises an immobilized BSA. In embodiments, Spot A1 of FIG. 39A comprises an immobilized S protein from SARS-CoV-2, Spot A2 of FIG. 39A comprises an immobilized N protein from SARS-CoV-2, Spot B1 of FIG. 39A comprises an immobilized S-RBD from SARS-CoV-2 strain 501Y.V2, and Spot B2 of FIG. 39A comprises an immobilized S protein from SARS-CoV-2 strain 501Y.V2.

In embodiments, each well comprises ten distinct binding domains. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 3 of FIG. 39B comprises an immobilized SARS-CoV-2 N protein, Spot 10 of FIG. 39B comprises an immobilized SARS-CoV-2 S-RBD, and Spots 2, 4, 5, 6, 7, 8, and 9 of FIG. 39B each comprises an immobilized BSA.

In embodiments, Spot 1 of FIG. 39B comprises an immobilized SARS-CoV-2 S protein, Spot 3 of FIG. 39B comprises an immobilized SARS-CoV-2 N protein. Spot 8 of FIG. 39B comprises an immobilized SARS-CoV-2 S-NTD, Spot 10 of FIG. 39B comprises an immobilized SARS-CoV-2 S-RBD, and Spots 2, 4, 5, 6, 7, and 9 of FIG. 39B each comprises an immobilized BSA.

In embodiments, Spot 1 of FIG. 39B comprises an immobilized wild-type S protein from SARS-CoV-2. Spot 2 of FIG. 39B comprises an immobilized Orf8 oligomer from SARS-CoV-2, Spot 3 of FIG. 39B comprises an immobilized N protein from SARS-CoV-2, Spot 4 of FIG. 39B comprises an immobilized Mem protein from SARS-CoV-2, Spot 6 of FIG. 39B comprises an immobilized Orf7a protein from SARS-CoV-2, Spot 7 of FIG. 39B comprises an immobilized Env protein from SARS-CoV-2, Spot 9 of FIG. 39B comprises an immobilized Orf8 monomer from SARS-CoV-2, Spot 10 of FIG. 39B comprises an immobilized wild-type S-RBD from SARS-CoV-2, Spot 5 of FIG. 39B comprises an immobilized BSA, and Spot 8 of FIG. 39B comprises an immobilized human serum albumin (HSA).

In embodiments, Spot 1 of FIG. 39B comprises an immobilized wild-type S protein from SARS-CoV-2, Spot 3 of FIG. 39B comprises an immobilized N protein from SARS-CoV-2, Spot 7 of FIG. 39B comprises an immobilized S protein from SARS-CoV-2 strain P.1. Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 2, 4, 5, 6, and 10 of FIG. 39B each comprises an immobilized BSA.

In embodiments, Spot 1 of FIG. 39B comprises an immobilized wild-type S protein from SARS-CoV-2. Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7. Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spots 4, 5, and 6 of FIG. 39B each comprises an immobilized BSA.

In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 4, 5, 6, and 10 of FIG. 39B each comprises an immobilized BSA.

In embodiments, Spot 1 of FIG. 39B comprises an immobilized wild-type S protein from SARS-CoV-2, Spot 2 of FIG. 39B comprises an immobilized S-RBD from SARS-CoV-2 strain 501Y.V2, Spot 3 of FIG. 39B comprises an immobilized N protein from SARS-CoV-2, Spot 4 of FIG. 39B comprises an immobilized S-RBD from SARS-CoV-2 strain P.1, Spot 6 of FIG. 39B comprises an immobilized S-RBD from SARS-CoV-2 strain B.1.1.7, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spot 5 of FIG. 39B comprises an immobilized BSA.

In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized influenza A H3 HA protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized MERS-CoV S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA

In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized MERS-CoV S protein, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein. Spot 6 of FIG. 39B comprises an immobilized SARS-CoV-2 S2, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized S protein from SARS-CoV-2. Spot 2 of FIG. 39B comprises an immobilized S1 from HCoV-NL63, Spot 3 of FIG. 39B comprises an immobilized N protein from SARS-CoV-2, Spot 4 of FIG. 39B comprises an immobilized S1 from SARS-CoV. Spot 6 of FIG. 39B comprises an immobilized S1 from SARS-CoV-2, Spot 7 of FIG. 39B comprises an immobilized S1 from HCoV-HKU1, Spot 8 of FIG. 39B comprises an immobilized S1 from HCoV-OC43, Spot 9 of FIG. 39B comprises an immobilized S1 from HCoV-229E, Spot 10 of FIG. 39B comprises an immobilized S-RBD from SARS-CoV-2, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized N protein from HCoV-NL63, Spot 3 of FIG. 39B comprises an immobilized N protein from SARS-CoV-2. Spot 4 of FIG. 39B comprises an immobilized N protein from SARS-CoV, Spot 6 of FIG. 39B comprises an immobilized N protein from MERS-CoV, Spot 7 of FIG. 39B comprises an immobilized N protein from HCoV-HKU1, Spot 8 of FIG. 39B comprises an immobilized N protein from HCoV-OC43, Spot 9 of FIG. 39B comprises an N protein from HCoV-229E, Spot 10 of FIG. 39B comprises an immobilized S-RBD from SARS-CoV-2, and Spot 5 of FIG. 39B comprises an immobilized BSA.

In embodiments, Spot 1 of FIG. 39B comprises an immobilized influenza B/Brisbane HA protein, Spot 2 of FIG. 39B comprises an immobilized influenza A/Shanghai H7 HA protein, Spot 4 of FIG. 39B comprises an immobilized influenza A/Michigan H1 HA protein, Spot 7 of FIG. 39B comprises an immobilized RSV pre-fusion F protein, Spot 8 of FIG. 39B comprises an immobilized influenza A/Hong Kong H3 HA protein, Spot 10 of FIG. 39B comprises an immobilized influenza B/Phuket HA protein, and Spots 3, 5, 6, and 9 of FIG. 39B each comprises an immobilized BSA.

In embodiments, about 1 μL to about 200 μL, about 3 μL to about 150 PL, about 5 μL to about 100 μL, about 10 μL to about 90 μL, about 15 μL to about 80 μL, about 20 μL to about 70 μL, about 30 μL to about 60 μL, about 50 μL, or about 150 μL of a blocking solution to each well of the plate. In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

1B. Preparation of reagents. In embodiments, the assay comprises measuring the amount of one or more calibration reagents. In embodiments, the calibration reagent comprises a known quantity of IgG and/or IgM. In embodiments, the calibration reagent comprises a blank solution containing no IgG or IgM. In embodiments, the assay comprises measuring the amount of multiple calibration reagents, e.g., at least 3, at least 4, at least 5, at least 6, at least 7, at least 8, at least 9, or at least 10 calibration reagents. In embodiments, the assay comprises generating a standard curve from the multiple calibration reagents. In embodiments, the multiple calibration reagents comprise a range of concentrations of IgG and/or IgM. In embodiments, the assay comprises diluting a concentration reagent to provide multiple calibration reagents comprising a range of concentrations. In embodiments, the calibration reagent is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. Calibration reagents are further described herein.

In embodiments, the assay comprises measuring the amount of one or more control reagents. In embodiments, the control reagent comprises a known quantity of IgG and/or IgM against the specific viral antigens in the assay, e.g., SARS-CoV-2 S. SARS-CoV-2 N, and/or SARS-CoV-2 S-RBD. In embodiments, the one or more control reagents comprises a first control reagent obtained from a subject known to never have been exposed to SARS-CoV-2, a second control reagent obtained from a subject during an early stage of infection by SARS-CoV-2, a third control reagent obtained from a subject during a late stage infection by SARS-CoV-2, a fourth control reagent obtained from a subject who has recovered from an infection by SARS-CoV-2, or a combination thereof. Control reagents are further described herein.

Examples of samples, e.g., biological samples, are provided herein. In embodiments, the sample is diluted about 2-fold, about 5-fold, about 10-fold, about 20-fold, about 30-fold, about 40-fold, about 50-fold, about 60-fold, about 70-fold, about 80-fold, about 90-fold, about 100-fold, about 250-fold, about 500-fold, about 750-fold, about 1000-fold, about 1500-fold, about 2000-fold, about 2500-fold, about 3000-fold, about 3500-fold, about 4000-fold, about 4500-fold, or about 5000-fold for use in the assay.

2. Addition of samples and reagents. In embodiments, the assay plate is washed at least once, at least twice, at least three times, at least four times, or at least five times with a wash buffer after incubation with the blocking solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the sample, one or more calibration reagents, and one or more control reagents are added to their respectively designated wells of the plate. In embodiments, about 5 μL to about 50 μL, about 10 μL to about 40 μL, about 20 μL to about 30 μL, about 15 μL, about 25 μL, or about 50 μL of the sample, calibration reagent, or control reagent is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 12 hours, or about 30 minutes to about 8 hours, or about 45 minutes to about 6 hours, or about 1 hour, or about 4 hours. In embodiments, the plate is incubated at about room temperature (e.g., about 22° C. to about 28° C.) while shaken at about 1500 rpm for about 4 hours. In embodiments, the plate is incubated at about room temperature (e.g., about 22° C. to about 28° C.) while shaken at about 700 rpm for about 1 hour.

3. Addition of detection reagent. In embodiments, the detection reagent is diluted from a stock solution of detection reagent to obtain a solution comprising a working concentration of detection reagent. Detection reagents are further described herein.

In embodiments, the assay plate is washed at least once, at least twice, at least three times, at least four times, or at least five times with a wash buffer after incubation with the sample, calibration reagent, or control reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the detection reagent solution is added to each well of the plate. In embodiments, about 5 μL to about 50 μL, about 10 μL to about 40 μL, about 10 μL to about 20 μL, about 20 μL to about 30 μL, about 15 μL, about 25 μL, or about 50 μL of the detection reagent solution is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) w % bile shaken at about 1500 rpm for about 1 hour. In embodiments, the plate is incubated at about room temperature (e.g., about 22° C. to about 28° C.) while shaken at about 700 rpm for about 1 hour.

4. Addition of read buffer. In embodiments, the assay plate is washed at least once, at least twice, at least three times, at least four times, or at least five times with a wash buffer after incubation with the detection reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, the read buffer is added to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well.

In embodiments, the assay comprises reading the plate, e.g., on a plate reader as described herein. In embodiments, the assay comprises reading the plate immediately following addition of the read buffer.

A further exemplary serology assay for detecting an antibody biomarker that binds to a SARS-CoV-2 antigen comprises:

(a) mixing (i) a coating solution comprising a binding reagent bound to a linking agent and (ii) a detection reagent, wherein each of the binding reagent and the detection reagent comprises a SARS-CoV-2 antigen, and wherein the detection reagent comprises a detectable label;

(b) contacting a surface with: (i) a sample comprising the antibody biomarker, (ii) a calibration reagent, or (iii) a control reagent, wherein the surface comprising one or more binding domains, wherein each binding domain comprises a targeting agent;

(c) contacting the surface with the mixture of (a);

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary serology assay for detecting an antibody biomarker that binds to a SARS-CoV-2 antigen comprises:

(a) mixing (i) a biotinylated binding reagent and (ii) a detection reagent, wherein each of the binding reagent and the detection reagent comprises a SARS-CoV-2 antigen, and wherein the detection reagent comprises a detectable label;

(b) contacting a surface with: (i) a sample comprising the antibody biomarker, (ii) a calibration reagent, or (iii) a control reagent, wherein the surface comprising one or more binding domains, wherein each binding domain comprises avidin or streptavidin:

(c) contacting the surface with the mixture of (a);

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

In embodiments, the surface is a multi-well plate. In embodiments, the assay further comprises a wash step prior to one or more of the assay steps. In embodiments, the wash step comprises washing the assay plate at least once, at least twice, at least three times, at least four times, or at least five times with a wash buffer. In embodiments, the assay plate is washed with at least about 10 μL, at least about 15 μL, at least about 20 μL, at least about 25 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, prior to step (a), a blocking solution is added to the plate to reduce non-specific binding of the coating solution or the biotinylated binding reagent to the surface. In embodiments, about 50 μL to about 250 μL, about 100 μL to about 200 μL, or about 150 μL of blocking solution is added per well of the plate. In embodiments, the plate is incubated at about 15° C. to about 30° C., about 18° C. to about 28° C., about 20° C. to about 26° C., or about 22° C. to about 24° C. In embodiments, the plate is incubated while shaken at about at about 500 rpm to about 2000 rpm, about 600 rpm to about 1500 rpm, or about 700 rpm to about 1000 rpm. In embodiments, the method comprises incubating the blocking solution on the plate for about 10 minutes to about 4 hours, about 20 minutes to about 3 hours, or about 30 minutes to about 2 minutes. In embodiments, the plate is incubated at about room temperature (e.g., about 22° C. to about 28° C.) while shaken at about 700 rpm for about 30 minutes to about 2 hours.

In embodiments comprising a coating solution, the assay further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL, or about 400 μL to about 600 μL of the coating solution. In embodiments, step (a) comprises incubating the linking agent and the binding reagent at about 15° C. to about 30° C., about 18° C. to about 28° C., about 20° C. to about 26° C. or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the assay further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C., about 18° C. to about 28° C., about 20° C. to about 26° C., or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, step (b) comprises adding about 5 μL to about 50 μL, about 10 μL to about 40 μL, about 20 μL to about 30 μL, about 15 μL, about 25 μL, about 35 μL, or about 50 μL of the sample, calibration reagent, or control reagent to each well of the plate. In embodiments, the plate is incubated at about 15° C. to about 30° C., about 18° C. to about 28 CC, about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (c) comprises adding about 5 μL to about 50 μL, about 10 μL to about 40 μL, about 10 μL to about 20 μL, about 20 μL to about 30 μL, about 15 μL, about 25 μL, about 35 μL, or about 50 μL of the mixture of (a) comprising the binding reagent and the detection reagent to each well of the plate. In embodiments, the plate is incubated at about 15° C. to about 30° C., about 18° C. to about 28° C., about 20° C. to about 26° C. or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

An exemplary multiplexed competitive serology assay detecting human neutralizing antibodies (also known as blocking antibodies) against SARS-CoV-2 antigens, as described in embodiments herein, comprises:

1A. Preparation of assay plate. In embodiments, the assay plate is a 384-well assay plate. In embodiments, the assay plate is a 96-well assay plate. In embodiments, each well comprises four distinct binding domains. In embodiments, the first binding domain comprises an immobilized SARS-CoV-2 S protein, the second binding domain comprises an immobilized SARS-CoV-2 N protein, and the third binding domain comprises an immobilized SARS-CoV-2 S-RBD. In embodiments, the fourth binding domain comprises a control protein that does not bind to human antibodies. In embodiments, the fourth binding domain comprises immobilized BSA. An embodiment of a well in a 384-well assay plate, comprising four binding domains (“spots”), is shown in FIG. 39A. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized SARS-CoV-2 S-RBD, and Spot B2 of FIG. 39A comprises an immobilized BSA. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized S-RBD from SARS-CoV-2 strain 501Y.V2, and Spot B2 of FIG. 39A comprises S protein from SARS-CoV-2 strain 501Y.V2.

In embodiments, each well comprises ten distinct binding domains. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 3 of FIG. 39B comprises an immobilized SARS-CoV-2 N protein, Spot 10 of FIG. 39B comprises an immobilized SARS-CoV-2 S-RBD, and Spots 2, 4, 5, 6, 7, 8, and 9 of FIG. 39B each comprises an immobilized BSA.

In embodiments, Spot 1 of FIG. 39B comprises an immobilized SARS-CoV-2 S protein. Spot 3 of FIG. 39B comprises an immobilized SARS-CoV-2 N protein, Spot 8 of FIG. 39B comprises an immobilized SARS-CoV-2 S-NTD, Spot 10 of FIG. 39B comprises an immobilized SARS-CoV-2 S-RBD, and Spots 2, 4, 5, 6, 7, and 9 of FIG. 39B each comprises an immobilized BSA.

In embodiments, Spot 1 of FIG. 39B comprises an immobilized wild-type S protein from SARS-CoV-2, Spot 2 of FIG. 39B comprises an immobilized an Orf8 oligomer from SARS-CoV-2, Spot 3 of FIG. 39B comprises an N protein from immobilized SARS-CoV-2, Spot 4 of FIG. 39B comprises an immobilized a Mem protein from SARS-CoV-2, Spot 6 of FIG. 39B comprises an immobilized an Orf7a protein from SARS-CoV-2, Spot 7 of FIG. 39B comprises an Env protein from SARS-CoV-2, Spot 9 of FIG. 39B comprises an Orf8 monomer from SARS-CoV-2, Spot 10 of FIG. 39B comprises a wild-type S-RBD from SARS-CoV-2, Spot 5 of FIG. 39B comprises an immobilized BSA, and Spot 8 of FIG. 39B comprises an immobilized human serum albumin (HSA).

In embodiments, Spot 1 of FIG. 39B comprises an immobilized wild-type S protein from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, and Spots 2, 4, 5, 6, and 10 of FIG. 39B each comprises an immobilized BSA.

In embodiments, Spot 1 of FIG. 39B comprises an immobilized wild-type S protein from SARS-CoV-2, Spot 2 of FIG. 39B comprises an S-D614G from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spots 4, 5, and 6 of FIG. 39B each comprises an immobilized BSA

In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 4, 5, 6, and 10 of FIG. 39B each comprises an immobilized BSA.

In embodiments, Spot 1 of FIG. 39B comprises an immobilized wild-type S protein from SARS-CoV-2, Spot 2 of FIG. 39B comprises an S-RBD from SARS-CoV-2 strain 501Y.V2, Spot 3 of FIG. 39B comprises an N protein from immobilized SARS-CoV-2, Spot 4 of FIG. 39B comprises an S-RBD from SARS-CoV-2 strain P.1, Spot 6 of FIG. 39B comprises an S-RBD from SARS-CoV-2 strain B.1.1.7, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spot 5 of FIG. 39B each comprises an immobilized BSA.

In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429. Spot 3 of FIG. 39B comprises an immobilized N protein from SARS-CoV-2, Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized influenza A H3 HA protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized MERS-CoV S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized SARS-CoV-2 S protein. Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized MERS-CoV S protein, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S2, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, Spot 1 of FIG. 39B comprises an immobilized S protein from SARS-CoV-2, Spot 2 of FIG. 39B comprises an immobilized an S1 from HCoV-NL63, Spot 3 of FIG. 39B comprises an immobilized N protein from SARS-CoV-2. Spot 4 of FIG. 39B comprises an immobilized S1 from SARS-CoV, Spot 6 of FIG. 39B comprises an immobilized S1 from SARS-CoV-2, Spot 7 of FIG. 39B comprises an immobilized S1 from HCoV-HKU1, Spot 8 of FIG. 39B comprises an immobilized S1 from HCoV-OC43, Spot 9 of FIG. 39B comprises an immobilized S1 from HCoV-229E. Spot 10 of FIG. 39B comprises an immobilized S-RBD from SARS-CoV-2, and Spot 5 of FIG. 39B comprises an immobilized BSA.

In embodiments, Spot 1 of FIG. 39B comprises an immobilized SARS-CoV-2 S protein, Spot 2 of FIG. 39B comprises an immobilized an N protein from HCoV-NL63, Spot 3 of FIG. 39B comprises an immobilized N protein from SARS-CoV-2, Spot 4 of FIG. 39B comprises an immobilized N protein from SARS-CoV. Spot 6 of FIG. 39B comprises an immobilized N protein from MERS-CoV, Spot 7 of FIG. 39B comprises an immobilized N protein from HCoV-HKU1, Spot 8 of FIG. 39B comprises an immobilized N protein from HCoV-OC43, Spot 9 of FIG. 39B comprises an N protein from HCoV-229E, Spot 10 of FIG. 39B comprises an immobilized S-RBD from SARS-CoV-2, and Spot 5 of FIG. 39B comprises an immobilized BSA.

In embodiments, Spot 1 of FIG. 39B comprises an immobilized influenza B/Brisbane HA protein, Spot 2 of FIG. 39B comprises an immobilized influenza A/Shanghai H7 HA protein. Spot 4 of FIG. 39B comprises an immobilized influenza A/Michigan H1 HA protein, Spot 7 of FIG. 39B comprises an immobilized RSV pre-fusion F protein, Spot 8 of FIG. 39B comprises an immobilized influenza A/Hong Kong H3 HA protein, Spot 10 of FIG. 39B comprises an immobilized influenza B/Phuket HA protein, and Spots 3, 5, 6, and 9 of FIG. 39B each comprises an immobilized BSA.

In embodiments, about 1 μL to about 200 μL, about 3 μL to about 150 μL, about 5 μL to about 100 μL, about 10 μL to about 90 μL, about 15 μL to about 80 μL, about 20 μL to about 70 μL, about 30 μL to about 60 μL, about 50 μL, or about 150 μL of a blocking solution to each well of the plate. In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

1B. Preparation of reagents. In embodiments, the assay comprises measuring the amount of one or more calibration reagents. In embodiments, the calibration reagent comprises a known quantity of IgG and/or IgM. In embodiments, the calibration reagent comprises a blank solution containing no IgG or IgM. In embodiments, the assay comprises measuring the amount of multiple calibration reagents, e.g., at least 3, at least 4, at least 5, at least 6, at least 7, at least 8, at least 9, or at least 10 calibration reagents. In embodiments, the assay comprises generating a standard curve from the multiple calibration reagents. In embodiments, the multiple calibration reagents comprise a range of concentrations of IgG and/or IgM. In embodiments, the assay comprises diluting a concentration reagent to provide multiple calibration reagents comprising a range of concentrations. In embodiments, the calibration reagent is diluted 1:10, 1:20, 1:30, 1:40, 1:50, 1:60, 1:70, 1:80, 1:90, 1:100, 1:140, 1:160, 1:200, 1:300, 1:400), 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. Calibration reagents are further described herein.

Examples of samples, e.g., biological samples, are provided herein. In embodiments, the sample is diluted about 2-fold, about 5-fold, about 10-fold, about 20-fold, about 30-fold, about 40-fold, about 50-fold, about 60-fold, about 70-fold, about 80-fold, about 90-fold, about 100-fold, about 250-fold, about 500-fold, about 750-fold, about 1000-fold, about 1500-fold, about 2000-fold, about 2500-fold, about 3000-fold, about 3500-fold, about 4000-fold, about 4500-fold, or about 5000-fold for use in the assay.

2. Addition of samples and reagents. In embodiments, the assay plate is washed at least once, at least twice, at least three times, at least four times, or at least five times with a wash buffer after incubation with the blocking solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the sample and one or more calibration reagents are added to their respectively designated wells of the plate. In embodiments, about 5 μL to about 50 μL, about 10 μL to about 40 μL, about 10 μL to about 20 μL, about 20 μL to about 30 μL, about 15 μL, about 25 μL, or about 50 μL of the sample or calibration reagent is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, or about 1200 rpm to about 1600 rpm. In embodiments, the plate is incubated for about 10 minutes to about 12 hours, or about 30 minutes to about 8 hours, or about 45 minutes to about 6 hours, or about 1 hour, or about 4 hours. In embodiments, the plate is incubated at about room temperature (e.g., about 22° C. to about 28° C.) while shaken at about 1500 rpm for about 4 hours. In embodiments, the plate is incubated at about room temperature (e.g., about 22° C. to about 28° C.) while shaken at about 700 rpm for about 1 hour.

3. Addition of ACE2 detection reagent. In embodiments, the ACE2 detection reagent is diluted from a stock solution of detection reagent to obtain a solution comprising a working concentration of ACE2 detection reagent. ACE2 is further described herein.

In embodiments, the assay plate is washed at least once, at least twice, at least three times, at least four times, or at least five times with a wash buffer after incubation with the sample or calibration reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the ACE2 detection solution is added to each well of the plate. In embodiments, about 5 μL to about 50 μL, about 10 μL to about 40 μL, about 10 μL to about 20 μL, about 20 μL to about 30 μL, about 25 μL, or about 50 μL of the ACE2 detection solution is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) while shaken at about 1500 rpm for about 1 hour. In embodiments, the plate is incubated at about room temperature (e.g., about 22° C. to about 28° C.) while shaken at about 700 rpm for about 1 hour.

4. Addition of read buffer. In embodiments, the assay plate is washed at least once, at least twice, at least three times, at least four times, or at least five times with a wash buffer after incubation with the ACE2 detection solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, the read buffer is added to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, or about 40 μL of the read buffer is added to each well.

In embodiments, the assay comprises reading the plate, e.g., on a plate reader as described herein. In embodiments, the assay comprises reading the plate immediately following addition of the read buffer.

A further exemplary competitive serology assay for detecting an antibody biomarker that binds to a SARS-CoV-2 antigen comprises:

(a) contacting a coating solution comprising a binding reagent bound to a linking reagent with a surface comprising one or more binding domains, wherein each binding domain comprises a targeting agent, and wherein the binding reagent is a SARS-CoV-2 antigen;

(b) contacting each binding domain with: (i) a sample comprising the antibody biomarker, (ii) a calibration reagent, or (iii) a control reagent;

(c) contacting each binding domain with an ACE2 detection reagent comprising a detectable label;

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary competitive serology assay for detecting an antibody biomarker that binds to a SARS-CoV-2 antigen comprises:

(a) contacting a biotinylated binding reagent with a surface comprising one or more binding domains, wherein each binding domain comprises avidin or streptavidin, and wherein the binding reagent is a SARS-CoV-2 antigen;

(b) contacting each binding domain with: (i) a sample comprising the antibody biomarker, (ii) a calibration reagent, or (iii) a control reagent;

(c) contacting each binding domain with an ACE2 detection reagent comprising a detectable label;

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

In embodiments, the surface is a multi-well plate. In embodiments, the assay further comprises a wash step prior to one or more of the assay steps. In embodiments, the wash step comprises washing the assay plate at least once, at least twice, at least three times, at least four times, or at least five times with a wash buffer. In embodiments, the assay plate is washed with at least about 10 μL, at least about 15 μL, at least about 20 μL, at least about 25 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer. In embodiments, the assay step does not comprise a wash step prior to any of steps (a), (b), or (c).

In embodiments comprising a coating solution, the assay further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL. or about 400 μL to about 600 μL of the coating solution. In embodiments, step (a) comprises incubating the linking agent and the binding reagent at about 15° C. to about 30° C., about 18° C. to about 28° C., about 20° C. to about 26° C., or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the assay further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C., about 18° C. to about 28° C., about 20° C. to about 26° C., or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, step (a) comprises adding about 10 μL to about 200 μL, about 5 μL to about 100 μL, about 10 μL to about 90 μL, about 15 μL to about 80 μL, about 20 μL to about 70 μL, about 30 μL to about 60 μL, or about 50 μL of the coating solution or a solution containing the biotinylated binding reagent to each well of the plate. In embodiments, the plate is incubated at about 15° C. to about 30° C. about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (b) comprises adding about 5 μL to about 50 μL, about 10 μL to about 40 μL, about 20 μL to about 30 μL, about 15 μL, about 25 μL, about 35 μL, or about 50 μL of the sample, calibration reagent, or control reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (c) comprises adding about 5 μL to about 50 μL, about 10 μL to about 40 μL, about 10 μL to about 20 μL, about 20 μL to about 30 μL, about 15 μL, about 25 μL, about 35 μL, or about 50 μL of a solution comprising the ACE2 detection reagent comprising the binding reagent and the detection reagent to each well of the plate. In embodiments, the plate is incubated at about 15° C. to about 30° C., about 18° C. to about 28° C. about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking. e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

### Quantitation Embodiments

In embodiments, the invention further provides a method of determining viral exposure in a subject, (a) comprising conducting an immunoassay method described herein on a biological sample of the subject; (b) detecting the virus, viral component, and/or biomarker (e.g., antibody biomarker or inflammatory or tissue damage biomarker) as described herein; (c) determining if the amount of detected virus, viral component, and/or biomarker is higher or lower relative to a control; and (d) determining the viral exposure of the subject based on the determination of (c). In embodiments, the method comprises normalizing the detected amount of biomarker (e.g., antibody biomarker) to a control and determining whether the subject is exposed to, infected by, and/or immune to the virus. In embodiments, the control is a biological sample containing a known amount of biomarkers (e.g., antibody biomarkers or inflammatory or tissue damage biomarkers). In embodiments, the control is a biological sample obtained from a subject known to have never been exposed to the virus. In embodiments, the control is a biological sample obtained from a subject known to have recovered from an infection by the virus. In embodiments, the virus is a coronavirus. In embodiments, the virus is SARS-CoV-2.

In embodiments comprising a multiplexed immunoassay for quantifying the amount of a biomarker (e.g., an antibody biomarker or an inflammatory or tissue damage biomarker), the method further comprises determining a threshold value of the biomarker in a healthy subject. In embodiments, the threshold value is determined from the aggregate results of measured biomarker amounts in multiple healthy subjects. For example, the aggregate results from a certain number of samples can determine the percentile, e.g., 99^{th} percentile or greater, of biomarker levels in a healthy subject. Various statistical models and algorithms can be utilized to calculate the extent of viral infection and/or degree of immunity in a subject by comparing and/or normalizing the subject's measured biomarker amount to the threshold value of that biomarker.

In embodiments, the multiplexed immunoassay for quantifying the amount of an antibody biomarker (e.g., a serology assay described herein) that specifically binds to a viral antigen is performed substantially with an assay that measures inhibition of binding between a viral protein and its associated host receptor, e.g., the binding of the coronavirus spike protein to the ACE2 receptor or the NRP1 receptor. In embodiments, the antibody biomarker inhibits binding between the viral protein and its associated host receptor. In embodiments, the inhibition assay indirectly detects the antibody biomarker. In embodiments, simultaneous direct detection (e.g., utilizing a viral antigen as a binding reagent) and indirect detection (e.g., measuring inhibition between a viral protein and its receptor) of the antibody biomarker improves specificity of the antibody biomarker detection.

In embodiments, the invention provides methods of assessing the affinity of an antibody biomarker to a viral antigen described herein, e.g., a SARS-CoV-2 antigen. As used in the context of antibodies, “affinity” refers to the strength of interaction between an epitope (e.g., on a viral antigen) and an antibody's antigen-binding site. In embodiments, the invention provides methods of assessing the binding kinetics between an antibody biomarker and viral antigen described herein. Methods of measuring antibody affinity and/or binding kinetics include, e.g., surface plasmon resonance (SPR) and bio-layer interference (BLI). Antibody affinity measurement is further described in, e.g., Underwood, Advances in Virus Research 34:283-309 (1988); Azimzadeh et al., J Mol Recognition 3(3):108-116 (1990); Hearty et al., Methods Mol Biol 907:411-442 (2012); and Singhal et al., Anal Chem 82(20):8671-8679 (2010).

In embodiments, the invention provides methods of assessing the affinity of a neutralizing antibody to a viral antigen described herein, e.g., a SARS-CoV-2 antigen. In embodiments, the affinity determination of a neutralizing antibody in a serum or plasma sample for SARS-CoV-2 comprises: a) titrating a labeled competitor to a surface comprising a known amount of SARS-CoV-2 S protein to determine the K_{dL} between the labeled ACE2 competitor and S protein; b) titrating: (i) a plasma sample known to contain neutralizing antibody for SARS-CoV-2 S while maintaining a constant ACE2 concentration, as described by equation 1(a); and (ii) ACE2 while maintaining a constant sample concentration, as described by equation 1(b); and c) solving the system of equations 1(a) and 1(b) to determine the average antibody concentration in sample [A] and average affinity K_{dA}. As described in equation 1(a), if the binding of fixed labeled ACE2, [L], is plotted against the log concentration of the unlabeled competitor antibody. [A], the resulting inhibition curve will be shifted by a factor of log(1+[L]/K_{dL}).

IC₅₀=K_{dLapp}=K_{dL}(1+[A]/K_{dA}) 1(a)

EC₅₀=K_{dAapp}=K_{dA}(1+[L]/K_{dL}) 1(b)

Affinity measurements are further described, e.g., in Hulme et al., British Journal of Pharmacology 161: 1219-1237 (2010).

Quantitation of Immobilized Binding Reagents

In embodiments, the invention further provides a method of determining uniformity of binding reagent immobilization on the surface. As used in the context of immobilizing reagents on surfaces, “uniformity” refers to an even distribution of reagent, e.g., binding reagent, on the surface. For example, in embodiments where the surface comprises a plurality of binding domains, the binding reagent should be uniformly distributed across the surface such that each binding domain has substantially the same number of binding reagents immobilized thereon. In embodiments, the surface is a plate, and the method comprises determining the intra-plate uniformity of binding reagent immobilization. As used herein, the term “intra-plate” refers to a comparison of different binding domains on the same plate (e.g., the amount of binding reagents immobilized in different binding domains on the same plate). In embodiments, the surface is a plate, and the method comprises determining the inter-plate uniformity of binding reagent immobilization. As used herein, the term “inter-plate” refers to a comparison between one or more different plates (e.g., the amount of binding reagents immobilized on different plates). Uniformity of binding reagents immobilization on the surface is important for quality control of the assays, e.g., ensuring producibility of experiments and reliable results.

In embodiments, the binding reagent comprises an epitope tag. As used herein, an “epitope tag” is a biological substance, such as a polypeptide or carbohydrate, which acts as an antigen that is recognized by an antibody. Epitope tags are known in the art. In embodiments, the epitope tag comprises a His tag, comprises maltose-binding protein (MBP), a V5 tag, a c-myc tag, an HA tag, a DYKDDDDK tag (SEQ ID NO: 548), a glutathione S-transfer (GST) tag, a Strep tag, an S-tag, a vesicular stomatitis virus glycoprotein (VSV-G) tag, a blue fluorescent protein (BFP) tag, a cyan fluorescent protein (CFP) tag, a green fluorescent protein (GFP) tag, a red fluorescent protein (RFP) tag, or a combination thereof. In embodiments, the His tag comprises about 4 to about 10 histidine residues, about 5 to about 8 histidine residues, or about 6 to about 7 histidine residues. In embodiments, the epitope tag is a His tag comprising six histidine residues (SEQ ID NO: 547), also referred to herein as a 6×His tag (SEQ ID NO: 547). In embodiments, the epitope tag on the binding reagent does not affect the binding of the binding reagent to its binding partner, e.g., virus, viral component, and/or biomarkers as described herein. In embodiments, the epitope tag on the binding reagent does not affect the immobilization of the binding reagent on to the surface, e.g., via targeting agent and targeting agent complement as described herein.

In embodiments, the binding reagent is immobilized at one or more distinct locations on a surface, and the method for determining uniformity of binding reagent immobilization on the surface comprises: (a) contacting the surface with an epitope tag-binding reagent, wherein the epitope tag-binding reagent binds specifically to the epitope tag on the immobilized binding reagent, and wherein the epitope tag-binding reagent comprises a detectable label; and (b) measuring the amount of detectable label at each of the one or more distinct locations on the surface, thereby determining the uniformity of binding reagent immobilization on the surface. In embodiments, the surface comprises a plurality of binding domains, and the method comprises measuring the amount of detectable label in each binding domain. In embodiments, the surface is a multi-well plate, and the method comprises measuring the amount of detectable label in each well. In embodiments, the epitope tag-binding reagent is an antibody, antigen, ligand, receptor, oligonucleotide, hapten, epitope, mimotope, or aptamer. In embodiments, the epitope tag-binding reagent is an antibody or antigen-binding fragment thereof. In embodiments, the epitope tag is a 6×His tag (SEQ ID NO: 547), and the epitope tag-binding reagent is an anti-6×His antibody.

In embodiments, the binding reagent is immobilized at one or more distinct locations on a surface, and the method for determining uniformity of binding reagent immobilization on the surface comprises: (a) contacting the surface with a calibration reagent, e.g., a pooled sample as described herein, wherein the calibration reagent is known to bind to the binding reagent; (b) contacting the surface with a detection reagent that binds the calibration reagent, thereby forming a binding complex at each of the one or more distinct locations on the surface; and (c) measuring the amount of binding complex at each of the one or more distinct locations on the surface, thereby determining the uniformity of binding reagent immobilization on the surface. In embodiments, the uniformity of binding reagent immobilization is determined by measuring the percent coefficient of variation (% CV) of the amount of binding complex at each of the one or more distinct locations on the surface. In embodiments, the calibration reagent comprises serum. In embodiments, the calibration reagent comprises plasma. Methods of measuring binding complexes are provided herein. In embodiments, the binding complex comprises a detectable label. In embodiments, the detection reagent of the binding complex comprises a detectable label.

Detectable labels are described herein and include, e.g., fluorescent label, colorimetric label, luminescent label, chemiluminescent label, electrochemiluminescent (ECL) label, bioluminescent label, phosphorescent label, and the like. In embodiments, the detectable label is an ECL label. In embodiments, the detectable label is an ECL label, and the measuring comprises visualizing the ECL signal intensity across the surface with a sensor, e.g., a charge-coupled device (CCD) sensor on a camera, to determine the uniformity of binding reagent immobilization.

In embodiments, the binding reagent immobilized on the surface is a viral antigen, e.g., for detecting an antibody biomarker as described herein. In embodiments, the binding reagent specifically binds to a viral component, e.g., for detecting a virus as described herein. In embodiments, the virus is a coronavirus. In embodiments, the virus is SARS-CoV-2. In embodiments, the binding reagent comprises the S, S1, S2, S-NTD, S-ECD, S-RBD, M, E, or N protein from SARS-CoV-2. In embodiments, the binding reagent comprises an antibody or antigen-binding fragment thereof that specifically binds to the S, S1, S2, S-NTD, S-ECD, S-RBD, M, E, or N protein from SARS-CoV-2.

### Assays for Viral Nucleic Acids Amplification and Detection of Viral Nucleic Acid

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus is SARS-CoV-2. In embodiments, the coronavirus nucleic acid is RNA. In embodiments, the binding reagent comprises a single stranded oligonucleotide.

In embodiments, the sample comprises a coronavirus nucleic acid. In embodiments, the method further comprises amplifying the coronavirus nucleic acid to form one or more additional copies of the coronavirus nucleic acid sequence, forming a plurality of binding complexes, each binding complex comprising a copy of the coronavirus nucleic acid sequence, and detecting the plurality of binding complexes, thereby detecting the coronavirus in the biological sample. In embodiments, the coronavirus nucleic acid is RNA, and the amplifying comprises reverse transcribing the RNA to form a cDNA, and amplifying the cDNA using polymerase chain reaction (PCR) to form a PCR product comprising a copy of the coronavirus nucleic acid sequence. In embodiments, the reverse transcription to form a cDNA and the PCR to amplify the cDNA are performed in a single reaction mixture. In embodiments, the reaction mixture further comprises a glycosylase enzyme. In embodiments, the glycosylase removes non-specific products from the reaction mixture. In embodiments, the glycosylase is uracil-N-glycosylase. In embodiments, the sample comprises an RT-PCR product, e.g., cDNA. In embodiments, the cDNA is generated from a coronavirus nucleic acid. In embodiments, the method comprises amplifying the cDNA using PCR to form a PCR product comprising a copy of the coronavirus nucleic acid sequence. In embodiments, the PCR is performed for about 10 to about 60 cycles, about 20 to about 50 cycles, or about 30 to about 40 cycles. In embodiments, the cDNA is amplified with a first primer that comprises a binding partner of the binding reagent and a second primer that comprises a detectable label or binding partner thereof, to form the PCR product. In embodiments, the first primer is a PCR forward primer and comprises the binding partner of the binding reagent at a 5′ end. In embodiments, the second primer is a PCR reverse primer and comprises the detectable label or binding partner thereof at a 3′ end. In embodiments, the PCR product comprises, in 5′ to 3′ order: the binding partner of the binding reagent, a copy of the coronavirus nucleic acid sequence, and the detectable label or binding partner thereof.

In embodiments, the first and second primers amplify a coronavirus nucleic acid sequence that encodes a protein, e.g., any of the coronavirus proteins described herein such as S, E, M, N, or a nonstructural protein. In embodiments, the first and second primers amplify a non-coding coronavirus nucleic acid sequence, i.e., that does not encode a gene. In embodiments, the first and second primers amplify a coronavirus nucleic acid sequence capable of identifying a coronavirus species. In embodiments, the coronavirus nucleic acid is SARS-CoV-2 RNA.

In embodiments, the method is a multiplexed method. In embodiments, the cDNA is amplified using multiple sets of primers, wherein each set of primers comprises a PCR forward primer and a PCR reverse primer as described herein. In embodiments, the PCR forward primer in each set of primers comprises a binding partner of the same binding reagent. In embodiments, the PCR forward primer in each set of primers comprises a binding partner of different binding reagents. In embodiments, each set of primers amplifies a different region of the cDNA to generate a plurality of PCR products, each having a different coronavirus nucleic acid sequence. For example, a first set of primers amplifies a region that encodes for the S protein, a second set of primers amplifies a region that encodes for the N protein, a third set of primers amplifies a region for a noncoding region, etc. In embodiments, each coronavirus nucleic acid sequence corresponds to a different binding reagent. In embodiments, the coronavirus nucleic acid sequence of the PCR product is identified by determining the binding reagent that binds the PCR product. In embodiments, the coronavirus nucleic acid is SARS-CoV-2 RNA.

In embodiments, the binding reagent comprises a single-stranded oligonucleotide, and the binding partner of the binding reagent comprises a complementary oligonucleotide of the binding reagent. In embodiments, the binding reagent further comprises a targeting agent complement. In embodiments, the targeting agent complement comprises an oligonucleotide that is complementary to a targeting agent on a surface, as described herein. In embodiments, the binding reagent is immobilized to the surface via the targeting agent-targeting agent complement interaction. In embodiments, each PCR product binds to a binding reagent to form one or more binding complexes on the surface. In embodiments comprising different binding reagents corresponding to different coronavirus nucleic acid sequences, each binding reagent is located at a distinct binding domain on the surface, and the detected coronavirus nucleic acid sequence is identified by the location of the binding complex on the surface.

In embodiments, the method comprises detecting the binding complex(es). In embodiments, the PCR product comprises a detectable label. In embodiments, the PCR product comprises a binding partner of a detectable label. Detectable labels are described herein. In embodiments, the detectable label is an electrochemiluminescence (ECL) label. In embodiments, the PCR product comprises biotin, and the detectable label comprises an ECL label linked to avidin or streptavidin. In embodiments, the PCR product comprises avidin or streptavidin, and the detectable label comprises an ECL label linked to biotin. Additional non-limiting examples of binding partners that can be on the detection probe and detectable label are provided herein.

An embodiment of the virus detection method is illustrated in FIG. 14. RNA is extracted from a sample containing an RNA virus (e.g., SARS-CoV-2), and the extracted RNA is converted to cDNA. A “Master Mix” is prepared by combining a forward primer comprising a 5′ binding reagent complement sequence and a cDNA complement sequence, a reverse primer comprising a cDNA reverse complement sequence and a 3′ binding partner of a detectable label, and other PCR components such as dNTPs and DNA polymerase (e.g., Taq polymerase). The cDNA and Master Mix are combined, and PCR is performed for 30 to 40 cycles to form a plurality of PCR products, each PCR product comprising the 5′ binding reagent complement sequence and 3′ binding partner of a detectable label. Each PCR product hybridizes to a binding reagent on a surface. The surface is then contacted with a detectable label, which binds to the PCR product. The PCR product bound to the detectable label is then subjected to detection as described herein.

In an alternative embodiment, the Master Mix comprises the components for performing the reverse transcription reaction and the PCR reaction, e.g., reverse transcriptase, DNA polymerase, forward and reverse primers, nucleotides, magnesium, ribonuclease inhibitor, and glycosylase, and the RNA extracted from the sample is added to the Master Mix, such that the reverse transcription reaction and the PCR reaction are performed with a single reaction mixture to form the PCR product. In embodiments, the single reaction mixture is: (1) incubated at a first temperature sufficient to activate the glycosylase; (2) incubated at a second temperature sufficient to perform the reverse transcription; and (3) incubated at temperature sufficient to perform PCR. In embodiments, the PCR product is bound to the surface and detected as described herein.

Cas Nickase Amplification and Detection of Viral Nucleic Acid

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus is SARS-CoV-2. In embodiments, the binding reagent comprises an oligonucleotide comprising a sequence complementary to the coronavirus nucleic acid sequence. In embodiments, the coronavirus nucleic acid is RNA. In embodiments, the binding reagent comprises a single stranded oligonucleotide. In embodiments, the detecting comprises directly detecting the binding complex. In embodiments, the detecting comprises detecting one or more components of the binding complex. e.g., the binding reagent.

In embodiments, the binding reagent comprises one or more of: a targeting agent complement, an amplification primer, a target hybridization region, an amplification blocker, and a secondary targeting agent complement. In embodiments, the binding reagent is an oligonucleotide comprising, in 5′ to 3′ order: a targeting agent complement, an amplification primer, a target hybridization region, an amplification blocker, and a secondary targeting agent complement.

In embodiments, the binding reagent comprises a targeting agent complement. In embodiments, the targeting agent complement comprises an oligonucleotide that is complementary to a targeting agent on a surface, as described herein. In embodiments, the method comprises immobilizing the binding reagent to the surface via the targeting agent-targeting agent complement interaction, prior to the detecting.

In embodiments, the binding reagent comprises an amplification primer. In embodiments, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. Non-limiting examples of isothermal amplification methods include helicase-dependent amplification and rolling circle amplification (RCA). In embodiments, the isothermal amplification method is RCA.

In embodiments, the binding reagent comprises a target hybridization region. In embodiments, the target hybridization region comprises an oligonucleotide that is complementary to the coronavirus nucleic acid.

In embodiments, the binding reagent comprises an amplification blocker. In embodiments, the amplification blocker comprises an oligonucleotide that blocks amplification of the amplification primer by preventing polymerase binding, inhibiting polymerase activity, and/or promoting polymerase dissociation. In embodiments, the amplification blocker comprises a nucleotide modification. Non-limiting examples of nucleotide modifications that block amplification include 3′-spacer C3, 3′-phosphate, 3′-dideoxy cytidine (3′-ddC), and 3′-inverted end. In embodiments, the amplification blocker comprises a secondary structure, e.g., a stem loop or a pseudoknot.

In embodiments, the target hybridization region and/or the amplification blocker comprises a target nucleic acid for a RNA-guided nickase. In embodiments, the nickase comprises a Cas9 nickase or a Cas12a (also known as Cpfl) nickase. Cas9 and Cas12a nickases are described in, e.g., Mali et al., Nat Biotechnol 31:833-838 (2013); Ran et al., Cell 155(2):479-480 (2013); Trevino et al., Methods Enzymol 546:161-174 (2014); and Fu et al., Nat Microbiol 4:888-897 (2019).

In embodiments, the RNA-guided nickase forms a complex with a guide RNA that hybridizes to a target nucleic acid (i.e., the nickase is “guided” to the target nucleic acid via complementarity between the guide RNA and the target nucleic acid). In embodiments, the target nucleic acid is double-stranded. In embodiments, the target nucleic acid comprises the hybridized binding reagent and coronavirus nucleic acid. In embodiments, the binding reagent and the coronavirus nucleic acid each forms one “strand” of a double-stranded target nucleic acid. In embodiments, the method comprises contacting the binding complex comprising the binding reagent and the coronavirus nucleic acid with the RNA-guided nickase. In embodiments, the nickase generates a single-stranded break in the binding reagent. In embodiments, the single-stranded break removes the amplification blocker from the binding reagent to form a cleaved binding reagent.

In embodiments, the cleaved binding reagent is not bound to the coronavirus nucleic acid, thereby allowing an additional copy of the binding reagent to bind to the coronavirus nucleic acid. In embodiments, the method further comprises repeating one or more steps to form a plurality of cleaved binding reagents. In embodiments, the method comprises detecting the plurality of cleaved binding reagents. In embodiments, the method comprises generating a plurality of cleaved binding reagents from a single copy of the coronavirus nucleic acid. In embodiments, forming the plurality of cleaved binding reagents amplifies the assay signal. In embodiments, the method has increased sensitivity of coronavirus detection as compared to a method that does not amplify the assay signal as described herein. In embodiments, the method is capable of detecting a lower amount of coronavirus nucleic acid in a biological sample as compared with a method that does not form the plurality of cleaved binding reagents, as described herein.

In embodiments, the binding reagent comprises a secondary targeting agent complement. In embodiments, the secondary targeting agent complement is a binding partner of a secondary targeting agent on a secondary surface. In embodiments, the secondary targeting agent and second targeting agent complement are substantially non-reactive with the targeting agent and targeting agent complement. For example, if the targeting agent and targeting agent complement are complementary oligonucleotides, then the secondary targeting agent and secondary targeting agent complement can be complementary oligonucleotides that do not hybridize to the targeting agent/targeting agent complement. In embodiments, the secondary targeting agent is streptavidin or avidin, and the secondary targeting agent complement is biotin. In embodiments, the secondary targeting agent is biotin, and the secondary targeting agent complement is streptavidin or avidin. In embodiments, the secondary targeting agent complement is adjacent to the amplification blocker on the binding reagent. In embodiments, cleavage of the binding reagent by the RNA-guided nickase removes the amplification blocker and secondary targeting complement from the binding reagent, to form a cleaved amplification blocker-secondary targeting agent complement.

In embodiments, a reaction mixture containing the plurality of cleaved binding reagents, uncleaved binding reagent, and cleaved amplification blocker-secondary targeting agent is formed. In embodiments, the method comprises removing the cleaved amplification blocker-secondary targeting complement and/or uncleaved binding reagent from the reaction mixture by contacting the reaction mixture with the secondary surface. In embodiments, the method has increased specificity as compared to a method that does not remove the amplification blocker and secondary targeting complement and/or uncleaved binding reagent from the reaction mixture.

In embodiments, the method comprises detecting the cleaved binding reagent(s) following removal of the amplification blocker-secondary targeting complement and/or uncleaved binding reagent. In embodiments, the detecting comprises contacting the reaction mixture with a surface comprising a targeting agent, thereby immobilizing the cleaved binding reagent(s) to the surface via hybridization of the targeting agent on the surface and the targeting agent complement on the cleaved binding reagent. In embodiments, the detecting further comprises binding the amplification primer to a template oligonucleotide and extending the amplification primer to form an extended sequence. In embodiments, the extending comprises polymerase chain reaction (PCR), ligase chain reaction (LCR), strand displacement amplification (SDA), self-sustained synthetic reaction (3SR), or an isothermal amplification method. In embodiments, the extending comprises an isothermal amplification method. In embodiments, the isothermal amplification method is RCA. In embodiments, the extended sequence binds an anchoring reagent immobilized on the surface. In embodiments, the coronavirus nucleic acid is detected and/or quantified by detecting or quantifying the amount of extended sequence bound to the surface as described herein. In embodiments, the surface is contacted with a labeled probe that binds to the extended sequence, wherein the labeled probe comprises a detectable label. In embodiments, the detectable label comprises an ECL label. Additional exemplary detectable labels are provided herein.

In embodiments, the cleaved binding reagent remains bound to the coronavirus nucleic acid. In embodiments, the method further comprises amplifying the coronavirus nucleic acid to form one or more additional copies of the coronavirus nucleic acid, forming a plurality of binding complexes with each copy of the coronavirus nucleic acid, and detecting the plurality of binding complexes, thereby detecting the coronavirus in the biological sample. In embodiments, the method comprises amplifying the coronavirus nucleic acid via the amplification primer on the cleaved binding reagent. In embodiments, the amplified coronavirus nucleic acid is contacted with an additional copy of the binding reagent, the binding complex formed therefrom is contacted with the RNA-guided nickase to cleave the binding reagent, and further amplifying the amplified coronavirus nucleic acid, thereby forming one or more additional copies of the coronavirus nucleic acid. In embodiments, the method comprises forming a plurality of binding complexes with the one or more additional copies of the coronavirus nucleic acid. In embodiments, the method comprises removing the uncleaved binding reagent and cleaved amplification blocker-secondary binding reagent as described herein. In embodiments, the method comprises detecting the plurality of binding complexes as described herein, thereby detecting the coronavirus in the biological sample. In embodiments, forming the additional copies of the coronavirus nucleic acid amplifies the assay signal. In embodiments, the method has increased sensitivity of coronavirus detection as compared to a method that does not amplify the assay signal as described herein. In embodiments, the method is capable of detecting a lower amount of coronavirus nucleic acid in a biological sample as compared with a method that does not form the one or more additional coronavirus nucleic acids and the plurality of binding complexes, as described herein.

An embodiment of the method is illustrated in FIG. 7. An oligonucleotide binding reagent comprises, in 5′ to 3′ order, a targeting agent complement, an amplification primer, a target hybridization region, an amplification blocker, and a secondary targeting agent complement (TAC). The binding reagent hybridizes with the analyte nucleic acid, e.g., coronavirus nucleic acid to form a binding complex. The binding complex is contacted with a Cas nickase, which nicks the binding reagent to remove the secondary TAC and amplification blocker, thereby activating the amplification primer for an amplification cycle. After one or more amplification cycles, the reaction mixture sample is incubated on a secondary surface comprising a secondary targeting agent, which removes any cleaved amplification blocker-secondary targeting agent complement and uncleaved binding reagent. Following the incubation on the secondary surface, the reaction mixture sample is then incubated on a surface comprising a targeting agent to immobilize the binding complex(es) onto the surface. The immobilized binding complex(es) is then subjected to extension and detection as described herein.

In an alternative embodiment of the method illustrated in FIG. 7, the binding complex comprising the binding reagent and analyte nucleic acid, e.g., coronavirus nucleic acid, is contacted with a Cas nickase. The Cas nickase nicks the binding reagent to remove the secondary TAC and amplification blocker to form a cleaved binding reagent, thereby activating the cleaved binding reagent for amplification and causes the cleaved binding reagent to dissociate from the analyte nucleic acid. In an “amplification cycle” of amplifying the number of cleaved binding reagents, the analyte nucleic acid binds to an additional copy of the binding reagent, which is cleaved by the Cas nickase to form an additional copy of the cleaved binding reagent activated for amplification. After one or more amplification cycles to form a plurality of cleaved binding reagents, the reaction mixture sample is incubated on a secondary surface comprising a secondary targeting agent, which removes any cleaved amplification blocker-secondary targeting agent complement and uncleaved binding reagent. Following the incubation on the secondary surface, the reaction mixture sample is then incubated on a surface comprising a targeting agent to immobilize the plurality of cleaved binding reagents onto the surface. The immobilized cleaved binding reagent(s) is then subjected to extension and detection as described herein.

Cas13 Detection of Viral RNA

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus is SARS-CoV-2. In embodiments, the binding reagent comprises an oligonucleotide comprising a sequence complementary to the coronavirus nucleic acid sequence. In embodiments, the coronavirus nucleic acid is RNA. In embodiments, the binding reagent comprises a single stranded oligonucleotide.

In embodiments, the binding reagent comprises one or more of: a targeting agent complement, an amplification primer, a ribonuclease recognition site, an amplification blocker, and a secondary targeting agent complement. In embodiments, the binding reagent is an RNA oligonucleotide comprising, in 5′ to 3′ order: a targeting agent complement, an amplification primer, a ribonuclease recognition site, and an amplification blocker. In embodiments, the binding reagent is an RNA oligonucleotide comprising, in 5′ to 3′ order: a targeting agent complement, an amplification primer, a ribonuclease recognition site, an amplification blocker, and a secondary targeting agent complement.

In embodiments, the binding reagent comprises a targeting agent complement. In embodiments, the targeting agent complement comprises an oligonucleotide that is complementary to a targeting agent on a surface, as described herein. In embodiments, the targeting agent complement is biotin, and the surface comprises streptavidin or avidin. In embodiments, the targeting agent complement is avidin or streptavidin, and the surface comprises biotin.

In embodiments, the binding reagent comprises an amplification primer. Amplification primers are described herein. In embodiments, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. Non-limiting examples of isothermal amplification methods include helicase-dependent amplification and rolling circle amplification (RCA). In embodiments, the isothermal amplification method is RCA.

In embodiments, the binding reagent comprises an amplification blocker. Amplification blockers are described herein. In embodiments, the amplification blocker comprises an oligonucleotide that blocks amplification of the amplification primer by preventing polymerase binding, inhibiting polymerase activity, and/or promoting polymerase dissociation. In embodiments, the amplification blocker comprises a nucleotide modification. Non-limiting examples of nucleotide modifications that block amplification include 3′-spacer C3, 3′-phosphate, 3′-dideoxy cytidine (3′-ddC), and 3′-inverted end. In embodiments, the amplification blocker comprises a secondary structure, e.g., a stem loop or a pseudoknot.

In embodiments, the method further comprises contacting the biological sample with a RNA-guided ribonuclease (RNase) prior to step (a). In embodiments, the RNA-guided ribonuclease is Cas13. In embodiments, the Cas13 is Cas13a, Cas13b, Cas13c, or Cas13d. In embodiments, the Cas13 is LwaCas13a, CcaCas13b, LbaCas13a, or PsmCas13b (see, e.g., Gootenberg, Abudayyeh et al., Science 356(6336):438-442 (2017)). Further non-limiting examples of Cas13 proteins are provided in Table 3. Cas13 proteins are further described in, e.g., Abudavyeh et al., Science 353(6299):aaf5573 (2016); Cox et al., Science 358(6366):1019-1027 (2017); O'Connell, J Mol Biol 431(1):66-87 (2019). Cas13 is known to exhibit “collateral” nuclease activity after recognition and cleavage of a target RNA, i.e., non-specific cleavage of any nearby RNA regardless of complementarity to the guide RNA (see, e.g., Gootenberg, Abudayyeh et al., Science 356(6336):438-442 (2017)).

TABLE 3
Exemplary Cas13 Proteins

| Bacterial Species | Protein Name |
|---|---|
| Prevotella sp. P5-125 (NZ_JXQL01000055.1) | PspCas13b |
| Capnocytophaga canimorsus Cc5 (NC_015846.1) | CcaCas13b |
| Alislipes sp. ZOR0009 (NZ_JTLD01000029.1) | AspCas13b |
| Prevotella sp. MA2016 (NZ_JHUW01000010.1) | PsmCas13b |
| Bergeyella TCC 43767 | BzoCas13b |
| (AG zoohelcum YA01000037.1) | |
| Riemerella anatipestifer | RanCas13b |
| Prevotella saccharolytica JCM 17484 | PsaCas13b |
| (NZ_BAKN101000001.1) | |
| Prevotella buccae ATCC 33574 (NZ_GL5863) | PbuCas13b |
| Prevotella intermedia ATCC 25611 | PinCas13b |
| (NZ_MEZ01000017.1) | |
| Prevotella intermedia (NZ_LBGT01000010.1) | Pin2Cas13b |
| Prevotella intermedia (NZ_AP014926.1) | Pin3Cas13b |
| Prevotella aurannaca JCM 15754 | PauCas13b |
| (NZ_BAKF01000019.1) | |
| Porphyromonas gulae (NZ_JRAL01000022.1) | PguCas13b |
| Porphyromonas gingivalis (NZ_CP0AJW4) | PigCas13b |

In embodiments, the binding reagent comprises a ribonuclease recognition site between the amplification primer and amplification blocker. In embodiments, the ribonuclease recognition site is an RNA dinucleotide. In embodiments, the ribonuclease is an RNA-guided ribonuclease. In embodiments, the ribonuclease is Cas13. Preferred RNA dinucleotides for cleavage by Cas13 are described in, e.g., Slaymaker et al., Cell Rep 26(13):3741-3751.e5 (2019); East-Seletsky et al., Mol Cell 66(3):373-383.e3 (2017); Gootenberg et al., Science 360(6387):439-444 (2018). For example, the preferred RNA dinucleotide for LwaCas13a, CcaCas13b, LbaCas13a, and PsmCas13b are AU, UC, AC, and GA, respectively.

In embodiments, the RNA-guided ribonuclease forms a complex with a guide RNA that hybridizes to a target coronavirus nucleic acid (i.e., the ribonuclease is “guided” to the target coronavirus nucleic acid). In embodiments, the RNA-guided ribonuclease cleaves the coronavirus nucleic acid. In embodiments, the binding reagent is added to the reaction mixture containing the RNA-guided ribonuclease and coronavirus nucleic acid after binding and cleavage of the coronavirus nucleic acid by the RNA-guided ribonuclease. In embodiments, the binding reagent is added to the reaction mixture containing the RNA-guided ribonuclease and coronavirus nucleic acid simultaneously or substantially simultaneously as binding and cleavage of the coronavirus nucleic acid by the RNA-guided ribonuclease. In embodiments, the coronavirus nucleic acid is SARS-CoV-2 RNA. In embodiments, the RNA-guided ribonuclease is Cas13.

In embodiments, the RNA-guided ribonuclease cleaves the binding reagent after binding and cleaving the coronavirus nucleic acid. In embodiments, the RNA-guided ribonuclease cleaves the binding reagent at the ribonuclease recognition site, thereby removing the amplification blocker from the binding reagent. In embodiments, the binding reagent and coronavirus nucleic acid form a binding complex before, during, or after the coronavirus nucleic acid or the binding reagent is cleaved by the RNA-guided ribonuclease. In embodiments, the coronavirus nucleic acid is SARS-CoV-2 RNA. In embodiments, the RNA-guided ribonuclease is Cas13.

In embodiments, the method further comprises contacting the binding reagent with a second ribonuclease, wherein the second ribonuclease is activated upon cleavage of the binding reagent and cleaves additional copies of the binding reagent. In embodiments, the binding reagent further comprises a second ribonuclease recognition site. In embodiments, the second ribonuclease is Csm6. Csm6 is a Type III CRISPR effector protein shown to be activated by the cleavage products of Cas13. Non-limiting examples of Csm6 proteins include EiCsm6, LsCsm6, and TtCsm6. See, e.g., Gootenberg et al., Science 360(6387):439-444 (2018). In embodiments, the second ribonuclease increases sensitivity of the method by increasing cleavage of the binding reagent to remove amplification blocker, thereby enabling amplification of the coronavirus nucleic acid.

In embodiments, the binding reagent comprises a secondary targeting agent complement. In embodiments, the secondary targeting agent complement is a binding partner of a secondary targeting agent on a secondary surface. In embodiments, the secondary targeting agent and second targeting agent complement are substantially non-reactive with the targeting agent and targeting agent complement. For example, if the targeting agent and targeting agent complement are complementary oligonucleotides, then the secondary targeting agent and secondary targeting agent complement can be complementary oligonucleotides that do not hybridize to the targeting agent/targeting agent complement. In embodiments, the secondary targeting agent complement is adjacent to the amplification blocker on the binding reagent. In embodiments, cleavage of the binding reagent by the RNA-guided nickase removes the amplification blocker and secondary targeting complement from the binding reagent, to form a cleaved amplification blocker-secondary targeting agent complement.

In embodiments, following the amplifying, a reaction mixture containing the of binding complexes, uncleaved binding reagent, and cleaved amplification blocker-secondary targeting agent is formed. In embodiments, the method comprises removing the cleaved amplification blocker-secondary targeting complement and/or uncleaved binding reagent from the reaction mixture by contacting the reaction mixture with the secondary surface. In embodiments, the method has increased specificity as compared to a method that does not remove the amplification blocker and secondary targeting complement and/or uncleaved binding reagent from the reaction mixture.

In embodiments, the method comprises detecting the binding complex comprising the coronavirus nucleic acid and the binding reagent. In embodiments, the detecting is performed after removal of the amplification blocker-secondary targeting complement and/or uncleaved binding reagent. In embodiments, the detecting comprises contacting the reaction mixture with a surface comprising a targeting agent, thereby immobilizing the binding complex to the surface via hybridization of the targeting agent on the surface and the targeting agent complement on the binding reagent. In embodiments, the detecting further comprises binding the amplification primer to a template oligonucleotide and extending the amplification primer to form an extended sequence. In embodiments, the extending comprises polymerase chain reaction (PCR), ligase chain reaction (LCR), strand displacement amplification (SDA), self-sustained synthetic reaction (3SR), or an isothermal amplification method. In embodiments, the extending comprises an isothermal amplification method. In embodiments, the isothermal amplification method is RCA. In embodiments, the extended sequence binds an anchoring reagent immobilized on the surface. In embodiments, the coronavirus nucleic acid is detected and/or quantified by detecting or quantifying the amount of extended sequence bound to the surface as described herein. In embodiments, the surface is contacted with a labeled probe that binds to the extended sequence, wherein the labeled probe comprises a detectable label. In embodiments, the detectable label comprises an ECL label. Additional exemplary detectable labels are provided herein. In embodiments, the coronavirus nucleic acid is SARS-CoV-2 RNA.

An embodiment of the method is illustrated in FIG. 8. An oligonucleotide binding reagent comprises, in 5 to 3′ order, a targeting agent complement, an amplification primer, a ribonuclease recognition site, and an amplification blocker. Cas13 forms a complex with the target RNA, e.g., coronavirus RNA, and the Cas13 cleaves the target RNA and indiscriminately cleaves the binding reagent to remove the amplification blocker, thereby activating the amplification primer. The reaction mixture sample is incubated on a surface comprising a targeting agent to immobilize the binding complex onto the surface. The immobilized binding complex is then subjected to extension and detection as described herein.

Detection of Viral Nucleic Acids and Single Nucleotide Polymorphisms (SNPs)

In embodiments, the invention provides a method for detecting a coronavirus nucleic acid in a biological sample. In embodiments, the invention provides a method of identifying the circulating strains of SARS-CoV-2 without sequencing a large number of SARS-CoV-2 isolates. Certain strains of SARS-CoV-2 are associated with increased transmissibility (e.g., the B.1.17, 501Y.V2, and P.1 strains) and diminished efficacy against currently available vaccines. In embodiments, the invention provides a method of real-time monitoring and assessing transmission patterns of SARS-CoV-2. In embodiments, the invention provides a method for determining a SARS-CoV-2 strain (e.g., the L strain or S strain, or the S-D614 or S-D614G strain, or the variants in Table 1A as described herein) in a biological sample. In embodiments, the method comprises an oligonucleotide ligation assay (OLA). OLA and other nucleic acid detection methods are described, e.g., in WO 2020/227016. In embodiments, the OLA method is used to detect, identify, and/or quantify a coronavirus nucleic acid (e.g., RNA). In embodiments, the coronavirus nucleic acid encodes the N gene. In embodiments, the coronavirus nucleic acid is the N1 region, N2 region, or N3 region of the N gene, as described herein. In embodiments, the OLA method is used to detect, identify, and/or quantify a single nucleotide polymorphism (SNP) at a polymorphic site in a coronavirus nucleic acid (e.g., RNA). In embodiments, the coronavirus is SARS-CoV-2. In embodiments, the method determines whether the SARS-CoV-2 genome location 8782 is 8782T (corresponding to the S strain) or 8782C (corresponding to the L strain). In embodiments, the method determines whether the SARS-CoV-2 genome location 28144 is 28144C (corresponding to the S strain) or 28144T (corresponding to the L strain). In embodiments, the method determines whether the SARS-CoV-2 genome location 23403 is 23403A (corresponding to the S-D614 strain) or 23403G (corresponding to the S-D614G strain). In embodiments, the method determines whether the SARS-CoV-2 genome location 11083 is 11083G or 11083T. In embodiments, the method determines whether the SARS-CoV-2 genome location 21765-21770, corresponding to amino acid residues 69-70 of the S protein, is deleted (corresponding to the B.1.1.7 strain). In embodiments, the method determines whether the SARS-CoV-2 genome location 22132 is 22132G (S protein R190) or 22132T (S protein R190S, corresponding to the P.1 strain). In embodiments, the method determines whether the SARS-CoV-2 genome location 22206 is 22206A (S protein D215) or 22206G (S protein D215G, corresponding to the 501Y.V2 strain). In embodiments, the method determines whether the SARS-CoV-2 genome location 22812 is 22812A (S protein K417) or 22812C (S protein K417T, corresponding to the P.1 strain). In embodiments, the method determines whether the SARS-CoV-2 genome location 22813 is 22813G (S protein K417) or 22813T (S protein K417N, corresponding to the 501Y.V2 strain). In embodiments, the method determines whether the SARS-CoV-2 genome location 22917 is 22917T (S protein L452) or 22917G (S protein L452R, corresponding to the Cal.20C strain). In embodiments, the method determines whether the SARS-CoV-2 genome location 23012 is 23012G (S protein E484) or 23012A (S protein E484K, corresponding to the 501Y.V2 and P.1 strains). In embodiments, the method determines whether the SARS-CoV-2 genome location 23063 is 23063A (S protein N501) or 23063T (S protein N501Y, corresponding to the B.1.1.7, 501Y.V2, and P.1 strains). In embodiments, the method determines whether the SARS-CoV-2 genome location 23403 is 23403A (S protein D614) or 23403G (S protein D614G, corresponding to the B.1.1.7, 501Y.V2, and P.1 strains). In embodiments, the method determines whether the SARS-CoV-2 genome location 23604 is 23604C (S protein P681) or 23604A (S protein P681H, corresponding to the B.1.1.7 strain). In embodiments, the method determines whether the SARS-CoV-2 genome location 23664 is 23664C (S protein A701) or 23664T (S protein A701V, corresponding to the 501Y.V2 strain). In embodiments, the method determines whether the SARS-CoV-2 genome location 22320 is 22320A (S protein D253) or 23664G (S protein D253G, corresponding to the B.1.526 strain). In embodiments, the method detects any of the SNPs as shown in Table 1A and Table 1C.

In embodiments, the OLA method for detecting a coronavirus nucleic acid comprises: (a) contacting the biological sample with: (i) a targeting probe, wherein the targeting probe is complementary to a first region of a target nucleic acid (e.g., the coronavirus nucleic acid or an RT-PCR product described herein), and wherein the targeting probe comprises an oligonucleotide tag; and (ii) a detection probe, wherein the detection probe is complementary to a second region that is adjacent to the first region of the target nucleic acid; (b) hybridizing the targeting and detection probes to the target nucleic acid; (c) ligating the targeting and detection probes that hybridize with perfect complementarity to the first and second regions of the target nucleic acid to form a ligated target complement comprising the oligonucleotide tag and the detectable label; (d) contacting the product of (c) with a surface comprising a binding reagent immobilized in one or more binding domains, wherein the binding reagent comprises an oligonucleotide complementary to the oligonucleotide tag; (e) forming a binding complex on the surface between the binding reagent and the ligated target complement; and (f) detecting the binding complex, thereby detecting, identifying, and/or quantifying the coronavirus nucleic acid. In embodiments, the coronavirus is SARS-CoV-2. In embodiments, the coronavirus nucleic acid is RNA. In embodiments, the sample comprises the coronavirus nucleic acid. In embodiments, the sample comprises an RT-PCR product, e.g., cDNA that is generated from the coronavirus nucleic acid.

In embodiments, the OLA method for detecting an SNP comprises: (a) contacting the biological sample with: (i) a targeting probe, wherein the targeting probe is complementary to a polymorphic site of target nucleic acid (e.g., the coronavirus nucleic acid or an RT-PCR product described herein), and wherein the targeting probe comprises an oligonucleotide tag; and (ii) a detection probe, wherein the detection probe is complementary to an adjacent region of the target nucleic acid containing the distinct SNP; (b) hybridizing the targeting and detection probes to the target nucleic acid; (c) ligating the targeting and detection probes that hybridize with perfect complementarity at the polymorphic site to form a ligated target complement comprising the oligonucleotide tag and the detectable label; (d) contacting the product of (c) with a surface comprising a binding reagent immobilized in one or more binding domains, wherein the binding reagent comprises an oligonucleotide complementary to the oligonucleotide tag; (e) forming a binding complex on the surface between the binding reagent and the ligated target complement; and (f) detecting the binding complex, thereby detecting, identifying, and/or quantifying the SNP at the polymorphic site of the coronavirus nucleic acid. In embodiments, the coronavirus is SARS-CoV-2. In embodiments, the coronavirus nucleic acid is RNA. In embodiments, the sample comprises the coronavirus nucleic acid. In embodiments, the sample comprises an RT-PCR product, e.g., cDNA that is generated from the coronavirus nucleic acid.

In embodiments, the ligating of the oligonucleotide probes is dependent on three events: (1) the targeting and detection probes must hybridize to complementary sequences within the target nucleic acid; (2) the targeting and detection probes must be adjacent to one another in a 5′- to 3′-orientation with no intervening nucleotides; and (3) the targeting and detection probes must have perfect base-pair complementarity with the target nucleic acid at the ligation site. A single nucleotide mismatch between the primers and target may inhibit ligation. In embodiments, the melting temperature (TM) of the oligonucleotide probes is about 55° C. to about 70° C., about 58° C. to about 68° C., about 60° C. to about 67° C., or about 62° C. to about 66° C. In embodiments, the ligation is performed at about 60° C. to about 70° C., about 61° C. to about 69° C., or about 62° C. to about 68° C. In embodiments, the ligation is performed at about 60° C., about 61° C., about 62° C., about 63° C., about 64° C., about 65° C., about 66° C., about 67° C., about 68° C., about 69° C., or about 70° C.

In embodiments, the targeting probe comprises, in 5′- to 3′-order: the oligonucleotide tag, and a sequence that is complementary to a first region of the target nucleic acid. In embodiments where the method detects a polymorphic site (SNP), the first region of the target nucleic acid comprises the polymorphic site. In embodiments, the oligonucleotide tag comprises a single-stranded oligonucleotide. In embodiments, the oligonucleotide tag does not hybridize with the target nucleic acid. In embodiments, the detection probe comprises, in 5′- to 3′-order: a sequence that is complementary to a second region of the target nucleic acid that is adjacent to the first region, and a detectable label or binding partner thereof. In embodiments, the 5′ end of the targeting probe is phosphorylated and is adjacent to the 3′ hydroxyl of the detection probe when the targeting and detection probes are hybridized to the target nucleic acid, such that the ends of the targeting and detection probes are ligated by formation of a phosphodiester bond. In embodiments, the 5′ end of the detection probe is phosphorylated and is adjacent to the 3′ hydroxyl of the targeting probe when the targeting and detection probes are hybridized to the target nucleic acid, such that the ends of the targeting and detection probes are ligated by formation of a phosphodiester bond.

In embodiments, the targeting and detection probes are ligated using a template-dependent ligase, for example, a DNA ligase such as E. coli DNA ligase, T4 DNA ligase, T. aquaticus (Taq) ligase, T. Thermophilus DNA ligase, or Pyrococcus DNA ligase. In embodiments, the ligase is a thermostable ligase. In embodiments, the targeting and detection probes are ligated by chemical ligation. In embodiments, the hybridization and ligation are performed in a combined step, for example, using multiple thermocycles and a thermostable ligase.

In embodiments where the method detects a coronavirus nucleic acid (e.g., the SARS-CoV-2 N gene or the N1, N2, and/or N3 regions thereof), the targeting probe hybridizes to the target nucleic acid such that the terminal 5′ nucleotide of the targeting probe hybridizes with the first region in the target nucleic acid, and the detection probe hybridizes to the second region in the target nucleic acid that is adjacent to the first region and provides a 3′ end for the ligation of the targeting and detection probes. In embodiments, the detection probe hybridizes to the target nucleic acid such that the terminal 5′ nucleotide of the detection probe hybridizes with the first region in the target nucleic acid, and the targeting probe hybridizes to the second region in the target nucleic acid that is adjacent to the first region and provides a 3′ end for the ligation of the targeting and detection probes. In embodiments, the detection probe hybridizes to the target nucleic acid such that the terminal 3′ nucleotide of the detection probe hybridizes with the first region in the target nucleic acid, and the targeting probe hybridizes to the second region of the target nucleic acid that is adjacent to the first region and provides a 5′ end for the ligation of the targeting and detection probes.

In embodiments where the method detects an SNP, the targeting probe hybridizes to the target nucleic acid such that the terminal 5′ nucleotide of the targeting probe hybridizes with the SNP in the target nucleic acid, and the detection probe hybridizes to the target nucleic acid adjacent to the SNP and provides a 3′ end for the ligation of the targeting and detection probes. In embodiments, the detection probe hybridizes to the target nucleic acid such that the terminal 5′ nucleotide of the detection probe hybridizes with the SNP in the target nucleic acid, and the targeting probe hybridizes to the target nucleic acid adjacent to the SNP and provides a 3′ end for the ligation of the targeting and detection probes. In embodiments, the detection probe hybridizes to the target nucleic acid such that the terminal 3′ nucleotide of the detection probe hybridizes with the SNP in the target nucleic acid, and the targeting probe hybridizes to the target nucleic acid adjacent to the SNP and provides a 5′ end for the ligation of the targeting and detection probes.

In embodiments, the method further comprises providing a blocking probe during the ligating of the targeting and detection probes. In embodiments, a blocking probe reduces non-specific bridging background during the ligation reaction. In embodiments, the blocking probe comprises a single stranded oligonucleotide that is complementary to the target nucleic acid and straddles the ligation site but does not comprise an oligonucleotide tag or a detectable label or binding partner thereof. In embodiments, the blocking probe comprises a single stranded oligonucleotide that is complementary to a probe designed to hybridize to the target nucleic acid. Without being bound by theory, it is believed that the presence of a blocking probe can reduce formation of complexes in which the target nucleic acid functions as a “bridge” for probes that are annealed to the target nucleic acid, but not ligated to one another, such that the complex can generate a false signal. In embodiments, a pair of blocking probes is provided during the ligating. In embodiments, one or more blocking probes is provided during the ligating in excess over the corresponding targeting and/or detection probes.

In embodiments, the detection probe comprises a detectable label. In embodiments, the detection probe comprises a binding partner of a detectable label. Detectable labels are described herein. In embodiments, the detectable label is an electrochemiluminescence (ECL) label. In embodiments, the detection probe comprises biotin, and the detectable label comprises an ECL label linked to avidin or streptavidin. In embodiments, the detection probe comprises avidin or streptavidin, and the detectable label comprises an ECL label linked to biotin. Additional non-limiting examples of binding partners that can be on the detection probe and detectable label are provided herein.

In embodiments, the target nucleic acid in the sample comprises a coronavirus nucleic acid. In embodiments, the target nucleic acid in the sample comprises an RT-PCR product, e.g., cDNA generated from the coronavirus nucleic acid. In embodiments, the method further comprises amplifying the target nucleic acid prior to contacting with the oligonucleotide probes. In embodiments, the method does not comprise amplifying the target nucleic acid. In embodiments, the nucleic acid is coronavirus RNA, and the method comprises reverse transcribing the coronavirus RNA into cDNA prior to step (a). In embodiments, the targeting probe and/or detection probe hybridize to the cDNA strand comprising the SNP of interest. In embodiments where the SNP of interest is in a protein coding sequence, the targeting probe and/or detection probe hybridize to the cDNA strand comprising the protein coding sequence. In embodiments, the targeting probe and/or detection probe hybridize to the cDNA strand comprising a complement of the SNP of interest. In embodiments wherein the SNP of interest is in a protein coding sequence, the targeting probe and/or detection probe hybridize to the cDNA strand comprising the complementary strand of the protein coding sequence. In embodiments, the coronavirus is SARS-CoV-2.

In embodiments, the region between SARS-CoV-2 genome locations 28250 and 28400, or between locations 28280 and 28390, or between locations 28300 and 28980, or between locations 28303 and 29374 is reverse transcribed prior to step (a). In embodiments, the region between SARS-CoV-2 genome locations 29000 and 29300, or between locations 29100 and 29280, or between locations 29150 and 29250, or between locations 29180 and 29246 is reverse transcribed prior to step (a). In embodiments, the region between SARS-CoV-2 genome locations 28500 and 28800, or between locations 28550 and 28790, or between locations 28600 and 28780, or between locations 28697 and 28768 is reverse transcribed prior to step (a). In embodiments, the cDNA formed by the reverse transcription is amplified by PCR. Exemplary PCR primers for amplification are shown in Table 20 and described in Lu et al., Emerg Infect Dis 26(8):1654-1665 (2020).

In embodiments, the region between SARS-CoV-2 genome locations 20000 and 25000, or between locations 21000 and 24500, or between locations 21500 and 24000, or between locations 21661 and 23812, or between locations 21739 and 23707, or between locations 21000 and 23000, or between 21500 and 22500, or between locations 21706 and 22341, or between locations 22000 and 24000, or between locations 22500 and 23500, or between locations 22624 and 23321, or between locations 23000 and 25000, or between locations 23000 and 24500, or between locations 23442 and 24103, or between locations 21662-21918, or between locations 22017-22341, or between locations 22851-23128, or between locations 23542-23813, or between locations 21706-21873, or between locations 22014-22252, or between locations 22624-22871, or between locations 22879-23099, or between locations 23296-23647, or between locations 23576-23815, or between locations 21600-21871, or between locations 22016-22331, or between locations 22849-23134, or between locations 23434-23755, or between locations 22589-22860, or between locations 22641-22865, or between locations 22561-22860, or between locations 22561-22865 is reverse transcribed prior to step (a). In embodiments, the cDNA formed by the reverse transcription is amplified by PCR. Exemplary PCR primers for amplification are shown in Table 25. In embodiments, the method comprises detecting an SNP in a synthetic oligonucleotide template. Exemplary synthetic oligonucleotide template sequences are shown in Table 23.

In embodiments, the region between SARS-CoV-2 locations 21661-23812 or locations 21739-23707 comprises the sequences encoding amino acid residues 69 to 701 of the S protein. In embodiments, the region between SARS-CoV-2 locations 21706-22341 comprises the sequences encoding amino acid residues 69 to 215 of the S protein. In embodiments, the region between SARS-CoV-2 locations 22624-23321 comprises the sequences encoding amino acid residues 417 to 501 of the S protein. In embodiments, the region between SARS-CoV-2 locations 23442-24103 comprises the sequences encoding amino acid residues 614 to 701 of the S protein. In embodiments, the region between SARS-CoV-2 locations 21662-21918 or locations 21706-21873, or locations 21600-21871 comprises the sequences encoding amino acid residues 69 to 70 of the S protein. In embodiments, the region between SARS-CoV-2 locations 22017-22341, or locations 22014-22252, or locations 22016-22331 comprises the sequences encoding amino acid residues 190 to 215 of the S protein. In embodiments, the region between SARS-CoV-2 locations 22851-23128, or locations 22879-23099, or locations 22849-23134 comprises the sequences encoding amino acid residues 452 to 501 of the S protein. In embodiments, the region between SARS-CoV-2 locations 23542-23813, or locations 23576-23815, or locations 23434-23755 comprises the sequences encoding amino acid residues 681 to 701 of the S protein. In embodiments, the region between SARS-CoV-2 locations 22624-22871, or locations 22589-22860, or locations 22641-22865, or locations 22561-22860, or locations 22561-22865 comprises the sequences encoding amino acid residue 417 of the S protein. In embodiments, the region between SARS-CoV-2 locations 23296-23647 comprises the sequences encoding amino acid residue 614 of the S protein.

In embodiments, the region surrounding location 8782 of the SARS-CoV-2 genome is reverse transcribed prior to step (a). In embodiments, the region surrounding location 28144 of the SARS-CoV-2 genome is reverse transcribed prior to step (a). In embodiments, the region surrounding location 23403 of the SARS-CoV-2 genome is reverse transcribed prior to step (a). In embodiments, the region surrounding location 11083 of the SARS-CoV-2 genome is reverse transcribed prior to step (a). In embodiments, the region surrounding location 21765-21770 of the SARS-CoV-2 genome is reverse transcribed prior to step (a). In embodiments, the region surrounding location 22132 of the SARS-CoV-2 genome is reverse transcribed prior to step (a). In embodiments, the region surrounding location 22206 of the SARS-CoV-2 genome is reverse transcribed prior to step (a). In embodiments, the region surrounding location 22812 of the SARS-CoV-2 genome is reverse transcribed prior to step (a). In embodiments, the region surrounding location 22813 of the SARS-CoV-2 genome is reverse transcribed prior to step (a). In embodiments, the region surrounding location 22917 of the SARS-CoV-2 genome is reverse transcribed prior to step (a). In embodiments, the region surrounding location 23012 of the SARS-CoV-2 genome is reverse transcribed prior to step (a). In embodiments, the region surrounding location 23063 of the SARS-CoV-2 genome is reverse transcribed prior to step (a). In embodiments, the region surrounding location 23403 of the SARS-CoV-2 genome is reverse transcribed prior to step (a). In embodiments, the region surrounding location 23604 of the SARS-CoV-2 genome is reverse transcribed prior to step (a). In embodiments, the region surrounding location 23664 of the SARS-CoV-2 genome is reverse transcribed prior to step (a). In embodiments, the region surrounding a SARS-CoV-2 genome location described in Table 1A is reverse transcribed prior to step (a). In embodiments, the region surrounding the SARS-CoV-2 S gene, N gene, E gene, 5′ UTR, nsp3 gene, Orf1ab gene, Orf1a gene, RdRp gene, Orf3a gene, Orf8 gene, or Orf10 gene is reverse transcribed prior to step (a). In embodiments, the region surrounding a particular genomic location includes about 10 to about 1000 nucleotides in length, about 20 to about 900 nucleotides in length, about 30 to about 800 nucleotides in length, about 40 to about 700 nucleotides in length, about 50 to about 600 nucleotides in length, about 60 to about 500 nucleotides in length, about 70 to about 400 nucleotides in length, about 80 to about 300 nucleotides in length, about 90 to about 200 nucleotides in length, or about 100 to about 150 nucleotides in length.

An embodiment of the OLA method for detecting an SNP described herein is represented schematically in FIG. 9. In FIGS. 9A-9C, a target nucleic acid (1) that comprises an SNP (2) is contacted with: a targeting probe (3) that comprises an oligonucleotide tag (4) and a sequence that is complementary to the SNP, and a detection probe (5) that comprises detectable label (6). The targeting and detection probes (3, 5) hybridize to the target nucleic acid, and the targeting and detection probes that hybridize with perfect complementarity at the SNP are ligated to form a ligated target complement (11) comprising the oligonucleotide tag and detectable label. The reaction mixture containing the ligated target complement is contacted with a surface comprising one or more binding reagents (7) immobilized in one or more binding domains (9). A signal (10) is detected if the ligated target complement is immobilized on the surface via hybridization of the complementary oligonucleotides in the oligonucleotide tag and the binding reagent. In FIG. 9D, the targeting probe has a mismatch with the SNP in the target nucleic acid, and thus, hybridization and ligation do not occur.

In embodiments, the method is a multiplexed OLA method. In embodiments where the method detects a coronavirus nucleic acid, the biological sample is contacted with one or more targeting probes and one or more detection probes to different regions of the coronavirus nucleic acid to form a plurality of ligated target complements. In embodiments, targeting probes for individual coronavirus nucleic acid regions comprise oligonucleotide tags corresponding to the individual coronavirus nucleic acid regions. In embodiments, the targeting probes for different coronavirus nucleic acid regions have substantially the same melting temperatures (T_{M}), e.g., within about 5° C., within about 4° C., within about 3° C., within about 2° C., or within about 1° C. In embodiments, the targeting probes for different coronavirus nucleic acid regions have substantially the same melting temperatures (T_{M}), e.g., within about 5° C. within about 4° C., within about 3° C., within about 2° C., or within about 1° C. In embodiments, the surface comprises a plurality of binding reagents capable of hybridizing to the different oligonucleotide tags. In embodiments, a plurality of binding complexes, each comprising a ligated target complement and its corresponding binding reagent, are formed on the surface, and the binding complexes are detected, thereby detecting, identifying, and/or quantifying each of the different coronavirus nucleic acid regions. In embodiments, the coronavirus is SARS-CoV-2. In embodiments, the corona-virus nucleic acid is RNA. In embodiments, the different corona-virus regions comprise the N1, N2, and N3 regions of SARS-CoV-2.

In embodiments where the method detects an SNP, the biological sample is contacted with one or more SNP-specific targeting probes and one or more detection probes to form a plurality of ligated target complements. In embodiments, the detection probes comprise identical sequences. In embodiments, each of the one or more SNP-specific targeting probes hybridizes to a different SNP at the target nucleic acid (e.g., SARS-CoV-28782T, 8782C, 28144C, 28144T, 23403A, 23403G, 11083G, 11083T, 21765-21770, 22132G, 22132T, 22206A, 22206G, 22812A, 22812C, 22813G, 22813T, 22917T, 22917G, 23012G, 23012A, 23063A, 23063T, 23403A, 23403G, 23604C, 23604A, 23664C, 23664T, 22320A, 22320G, or any of the genome locations and SNPs in Tables 1A and 1C herein). In embodiments, the targeting probe and detection probe for detecting a SNP in Tables 1A and 1C comprises a sequence described in Table 22. In embodiments, the blocking oligonucleotide for detecting the SNPs in Tables 1A and 1C comprises a sequence described in Table 24. In embodiments, targeting probes for different SNPs comprise different oligonucleotide tags. In embodiments, the targeting probes for different SNPs have substantially the same melting temperatures (T_{M}), e.g., within about 5° C. within about 4° C., within about 3° C., within about 2° C., or within about PC. In embodiments, the surface comprises a plurality of binding reagents capable of hybridizing to the different oligonucleotide tags. In embodiments, a plurality of binding complexes, each comprising a ligated target complement and its corresponding binding reagent, are formed on the surface, and the binding complexes are detected, thereby detecting, identify, and/or quantifying each of the SNPs at the polymorphic site of the coronavirus nucleic acid. In embodiments, the coronavirus is SARS-CoV-2. In embodiments, the coronavirus nucleic acid is RNA.

In embodiments, the multiplexed OLA method simultaneously detects at least 2, at least 3, at least 4, at least 5, at least 6, at least 7, at least 8, at least 9, or at least 10 coronavirus nucleic acids as described herein, e.g., the SARS-CoV-2 N1, N2, and N3 regions. In embodiments, the multiplexed OLA method simultaneously detects at least 2, at least 3, at least 4, at least 5, at least 6, at least 7, at least 8, at least 9, or at least 10 SNPs, e.g., the SNPs at SARS-CoV-2 genome locations 8782, 11083, 23403, and 28144. In embodiments, the multiplexed OLA method simultaneously detects the SNPs at SARS-CoV-2 genome locations 21765-21770, 22132, 22206, 22813, 22812, 22917, 23012, 23063, 23604, and 23664, which correspond to amino acid residues 69-70, R190, D215, K417, K417, L452, E484, N501, P681, and A701, respectively, of the SARS-CoV-2 S protein. In embodiments, the multiplexed OLA method comprises contacting the biological sample with a surface comprising at least 2, at least 3, at least 4, at least 5, at least 6, at least 7, at least 8, at least 9, or at least 10 distinct binding domains, wherein each binding domain comprises a unique binding reagent, each unique binding reagent capable of recognizing a different oligonucleotide tag as described herein.

In embodiments, the targeting probe for the SARS-CoV-2 N1 region comprises SEQ ID NO:22, 24, or 25. In embodiments, the detection probe for SARS-CoV-2 N1 region comprises SEQ ID NO:23 or 26. In embodiments, the blocking oligonucleotide for SARS-CoV-2 N1 region comprises any one of SEQ ID NOs:39-42. In embodiments, the targeting probe for the SARS-CoV-2 N2 region comprises SEQ ID NO:27 or 29. In embodiments, the detection probe for the SARS-CoV-2 N2 region comprises SEQ ID NO:28 or 30. In embodiments, the blocking oligonucleotide for SARS-CoV-2 N2 region comprises any one of SEQ ID NO:43-46. In embodiments, the targeting probe for the SARS-CoV-2 N3 region comprises SEQ ID NO:31 or 33. In embodiments, the detection probe for the SARS-CoV-2 N3 region comprises SEQ ID NO:32 or 34. In embodiments, the blocking oligonucleotide for SARS-CoV-2 N3 region comprises any one of SEQ ID NOs:47-50.

In embodiments, the method further comprises detecting a control gene. In embodiments, the control gene comprises an endogenous gene of the subject from which the biological sample was obtained. In embodiments, the control gene comprises the human RPP30 gene. In embodiments, the targeting probe for the human RPP30 gene comprises SEQ ID NO:35 or 37. In embodiments, the detection probe for the human RPP30 gene comprises SEQ ID NO:36 or 38. In embodiments, the blocking probe for the human RPP30 gene comprises any one of SEQ ID NOs:51-54.

In embodiments, a multiplexed OLA method for SARS-CoV-2 N1, N2, N3, and human RPP30 gene is conducted in a multi-well plate, wherein each well comprises ten binding domains (“spots”) as shown in FIG. 39B. In embodiments, Spot 4 of FIG. 39B comprises a binding reagent for a ligated target complement to the SARS-CoV-2 N1 region, Spot 5 of FIG. 39B comprises a binding reagent for a ligated target complement to the SARS-CoV-2 N2 region, Spot 9 of FIG. 39B comprises a binding reagent for a ligated target complement to the SARS-CoV-2 N3 region, and Spot 10 of FIG. 39B comprises a binding reagent for a ligated target complement of the human RPP30 gene.

In embodiments, the targeting probe for the SARS-CoV-2 S strain comprises SEQ ID NO:1 or 3. In embodiments, the targeting probe for the SARS-CoV-2 L strain comprises SEQ ID NO:2 or 4. In embodiments, the targeting probe for the SARS-CoV-2 S-D614 strain comprises SEQ ID NO:15. In embodiments, the targeting probe for the SARS-CoV-2 S-D614G strain comprises SEQ ID NO:16. In embodiments, the detection probe for SARS-CoV-2 comprises SEQ ID NO:5, 6, or 17. In embodiments, the blocking probe for SARS-CoV-2 location 8782 comprises SEQ ID NO:7 or 8. In embodiments, the blocking probe for SARS-CoV-2 location 28144 comprises SEQ ID NO:9 or 10. In embodiments, the blocking probe for SARS-CoV-2 location 23403 comprises SEQ ID NO:18 or 19. In embodiments, the primer for reverse transcribing the region surrounding SARS-CoV-2 genome location 8782 comprises SEQ ID NO:11 or 12. In embodiments, the primer for reverse transcribing the region surrounding SARS-CoV-2 genome location 28144 comprises SEQ ID NO:13 or 14. In embodiments, the primer for reverse transcribing the region surrounding SARS-CoV-2 genome location 23403 comprises SEQ ID NO:20 or 21.

In embodiments, the OLA method for detecting an SNP at SARS-CoV-2 genome locations 8782, 23403, 28144, and/or 11083, e.g., as described herein, is conducted in a multi-well plate, wherein each well comprises ten binding domains (“spots”) as shown in FIG. 39B. In embodiments, each binding domain comprises a binding reagent capable of hybridizing to an oligonucleotide tag on a targeting probe, as described herein. In embodiments, the oligonucleotide tag of SEQ ID NO:1 or 492 is capable of hybridizing to the binding reagent in Spot 1 of FIG. 39B. In embodiments, the oligonucleotide tag of SEQ ID NO:2 or 493 is capable of hybridizing to the binding reagent in Spot 6 of FIG. 39B. In embodiments, the oligonucleotide tag of SEQ ID NO:3 or 495 is capable of hybridizing to the binding reagent in Spot 2 of FIG. 39B. In embodiments, the oligonucleotide tag of SEQ ID NO:4 or 4% is capable of hybridizing to the binding reagent in Spot 7 of FIG. 39B. In embodiments, the oligonucleotide tag of SEQ ID NO:15 or 198 is capable of hybridizing to the binding reagent in Spot 3 of FIG. 39B. In embodiments, the oligonucleotide tag of SEQ ID NO:16 or 499 is capable of hybridizing to the binding reagent in Spot 8 of FIG. 39B. In embodiments, the oligonucleotide tag of SEQ ID NO:501 or 504 is capable of hybridizing to the binding reagent in Spot 4 of FIG. 39B. In embodiments, the oligonucleotide tag of SEQ ID NO:502 or 505 is capable of hybridizing to the binding reagent in Spot 9 of FIG. 39B. In embodiments, the oligonucleotide tag of SEQ ID NO:534 or 537 is capable of hybridizing to the binding reagent in Spot 4 of FIG. 39B. In embodiments, the oligonucleotide tag of SEQ ID NO:533 or 536 is capable of hybridizing to the binding reagent in Spot 10 of FIG. 39B.

In embodiments, the invention provides a method for detecting a coronavirus nucleic acid in a biological sample. In embodiments, the method comprises: (a) contacting the biological sample with (i) a polymerase, (ii) a forward primer, wherein the forward primer binds to a first region of a target nucleic acid (e.g., the coronavirus nucleic acid or an RT-PCR product described herein), and wherein the forward primer comprises an oligonucleotide tag; and (iii) a reverse primer, wherein the reverse primer binds to a second region of the target nucleic acid; (b) amplifying the target nucleic acid using the polymerase to form an amplified target nucleic acid comprising the oligonucleotide tag; (c) hybridizing the amplified target nucleic acid with an internal detection probe that is complementary to at least a portion of the amplified target nucleic acid, thereby forming a hybridized target; (d) contacting the hybridized target with a surface comprising a binding reagent immobilized in one or more binding domains, wherein the binding reagent comprises an oligonucleotide complementary to the oligonucleotide tag; (e) forming a binding complex on the surface between the binding reagent and the hybridized target; and (f) detecting the binding complex, thereby detecting, identifying, and/or quantifying the coronavirus nucleic acid. In embodiments, the coronavirus is SARS-CoV-2. In embodiments, the coronavirus nucleic acid is RNA. In embodiments, the target nucleic acid is the N1, N2, and/or N3 regions of SARS-CoV-2. In embodiments, the sample comprises the coronavirus nucleic acid. In embodiments, the sample comprises an RT-PCR product, e.g., cDNA that is generated from the coronavirus nucleic acid.

In embodiments, the method further comprises amplifying the target nucleic acid prior to contacting with the oligonucleotide probes. In embodiments, the nucleic acid is coronavirus RNA, and the method comprises reverse transcribing the coronavirus RNA into cDNA prior to step (a). In embodiments, the coronavirus is SARS-CoV-2. In embodiments, the method comprises, prior to step (a), reverse transcribing the region between SARS-CoV-2 genome locations 28250 and 28400, or between locations 28280 and 28390, or between locations 28300 and 28980, or between locations 28303 and 29374. In embodiments, the method comprises, prior to step (a), reverse transcribing the region between SARS-CoV-2 genome locations 29000 and 29300, or between locations 29100 and 29280, or between locations 29150 and 29250, or between locations 29180 and 29246. In embodiments, the method comprises, prior to step (a), reverse transcribing the region between SARS-CoV-2 genome locations 28500 and 28800, or between locations 28550 and 28790, or between locations 28600 and 28780, or between locations 28697 and 28768.

In embodiments, the internal detection probe comprises a detectable label. In embodiments, the internal detection probe comprises a binding partner of a detectable label. Detectable labels are described herein. In embodiments, the detectable label is an electrochemiluminescence (ECL) label. In embodiments, the internal detection probe comprises biotin, and the detectable label comprises an ECL label linked to avidin or streptavidin. In embodiments, the internal detection probe comprises avidin or streptavidin, and the detectable label comprises an ECL label linked to biotin. Additional non-limiting examples of binding partners that can be on the internal detection probe and detectable label are provided herein.

In embodiments, the forward primer for SARS-CoV-2 N1 region comprises SEQ ID NO:55. In embodiments, the reverse primer for SARS-CoV-2 N1 region comprises SEQ ID NO:56. In embodiments, the internal detection probe for SARS-CoV-2 N1 region comprises SEQ ID NO:57. In embodiments, the forward primer for SARS-CoV-2 N2 region comprises SEQ ID NO:58. In embodiments, the reverse primer for SARS-CoV-2 N2 region comprises SEQ ID NO:59. In embodiments, the internal detection probe for SARS-CoV-2 N2 region comprises SEQ ID NO:60. In embodiments, the forward primer for SARS-CoV-2 N3 region comprises SEQ ID NO:61. In embodiments, the reverse primer for SARS-CoV-2 N3 region comprises SEQ ID NO:62. In embodiments, the internal detection probe for SARS-CoV-2 N3 region comprises SEQ ID NO:63.

In embodiments, the method further comprises detecting a control gene. In embodiments, the control gene comprises an endogenous gene of the subject from which the biological sample was obtained. Suitable control genes are known to those of skill in the art. In embodiments, the control gene comprises the human RPP30 gene. In embodiments, the forward primer for the human RPP30 gene comprises SEQ ID NO:64. In embodiments, the reverse primer for the human RPP30 gene comprises SEQ ID NO:65. In embodiments, the internal detection probe for the human RPP30 gene comprises SEQ ID NO:66.

In embodiments, a multiplexed method for SARS-CoV-2 N1, N2, N3, and human RPP30 gene is conducted in a multi-well plate, wherein each well comprises ten binding domains (“spots”) as shown in FIG. 39B. In embodiments, Spot 8 of FIG. 39B comprises a binding reagent for a hybridized target comprising the SARS-CoV-2 N1 region, Spot 9 of FIG. 39B comprises a binding reagent for a hybridized target comprising the SARS-CoV-2 N2 region, Spot 10 of FIG. 39B comprises a binding reagent for a hybridized target comprising the SARS-CoV-2 N3 region, and Spot 1 of FIG. 39B comprises a binding reagent for a hybridized target comprising the human RPP30 gene.

In embodiments, the invention provides a multiplexed OLA method for detecting SARS-CoV-2 strains, comprising detecting the SARS-CoV-2 genome location 21765-21770 (corresponding to amino acid residues 69-70 of the S protein), location 22132 (corresponding to amino acid residue 190 of the S protein), location 22206 (corresponding to amino acid residue 215 of the S protein), location 22812 (corresponding to amino acid residue 417 of the S protein), location 22813 (corresponding to amino acid residue 417 of the S protein), location 22917 (corresponding to amino acid residue 452 of the S protein), location 23012 (corresponding to amino acid residue 484 of the S protein), location 23063 (corresponding to amino acid residue 501 of the S protein), location 23403 (corresponding to amino acid residue 614 of the S protein), location 23604 (corresponding to amino acid residue 681 of the S protein), and/or location 23664 (corresponding to amino acid residue 701 of the S protein). In embodiments, the method further comprises detecting a control gene. In embodiments, the control gene comprises a gene or region that is conserved across SARS-CoV-2 strains. In embodiments, the control gene is SARS-CoV-2 N1.

In embodiments, the multiplexed OLA method detects: a deletion at SARS-CoV-2 genome location 21765-21770 (corresponding to a deletion of residues 69-70 of the S protein), a G>T SNP at SARS-CoV-2 genome location 22132 (corresponding to an R190S mutation in the S protein), an A>G SNP at SARS-CoV-2 genome location 22206 (corresponding to a D215G mutation in the S protein), an A>G SNP at SARS-CoV-2 genome location 22320 (corresponding to a D253G mutation in the S protein), an A>C SNP at SARS-CoV-2 genome location 22812 (corresponding to a K417T mutation in the S protein), a G>T SNP at SARS-CoV-2 genome location 22813 (corresponding to a K417N mutation in the S protein), a T>G SNP at SARS-CoV-2 genome location 22917 (corresponding to an L452R mutation in the S protein), a G>A SNP at SARS-CoV-2 genome location 23012 (corresponding to a E484K mutation in the S protein), an A>T SNP at SARS-CoV-2 genome location 23063 (corresponding to a N501Y mutation the S protein), an A>G SNP at SARS-CoV-2 genome location 23403 (corresponding to a D614G mutation in the S protein), a C>A SNP at SARS-CoV-2 genome location 23604 (corresponding to a P681H mutation in the S protein), and/or a C>T SNP at SARS-CoV-2 genome location 23664 (corresponding to an A701V mutation in the S protein). In embodiments, the multiplexed OLA method detects any combination of the SNPs in Table 1A.

In embodiments, the multiplexed OLA method detects multiple variants of SARS-CoV-2. In embodiments, the multiplexed OLA method detects multiple variants of SARS-CoV-2. As used herein, “variant” refers to a strain that has one or more mutations relative to the SARS-CoV-2 reference strain NC_045512. In embodiments, the multiplexed OLA method is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein A69-70 deletion); Spot 2 comprises a binding reagent for detecting 22132T (S protein R190S mutation); Spot 3 comprises a binding reagent for detecting 22206G (S protein D215G mutation); Spot 4 comprises a binding reagent for detecting 22917G (S protein L452R mutation); Spot 6 comprises a binding reagent for detecting 23012A (S protein E484K mutation); Spot 7 comprises a binding reagent for detecting 23063T (S protein N501Y mutation); Spot 8 comprises a binding reagent for detecting 23403G (S protein D614G mutation); Spot 9 comprises a binding reagent for detecting 23604A (S protein P681H mutation); Spot 10 comprises a binding reagent for detecting 23664T (S protein A701V mutation); and Spot 5 comprises a binding reagent for detecting a control gene of SARS-CoV-2 that is conserved across all SARS-CoV-2 strains, e.g., the N1 gene.

In embodiments, the multiplexed OLA method simultaneously detects the reference SARS-CoV-2 strain and one or more variants, e.g., by detecting both the wild-type nucleotide and the variant SNP at a genome location. In embodiments, the multiplexed OLA method is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein A69-70 deletion), and Spot 6 comprises a binding reagent for detecting the wild-type sequence at 21765-21770 (S protein residues 69-70); Spot 2 comprises a binding reagent for detecting 22132T (S protein R190S mutation), and Spot 7 comprises a binding reagent for detecting 22132G (S protein R190); Spot 3 comprises a binding reagent for detecting 22206G (S protein D215G mutation), and Spot 8 comprises a binding reagent for detecting 22206A (S protein D215); Spot 4 comprises a binding reagent for detecting 22917G (S protein L452R mutation), and Spot 9 comprises a binding reagent for detecting 22917T (S protein L452).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting 23012G (S protein E484), and Spot 6 comprises a binding reagent for detecting 23012A (S protein E484K mutation); Spot 2 comprises a binding reagent for detecting 23063A (S protein N501), and Spot 7 comprises a binding reagent for detecting 23063T (S protein N501Y mutation); Spot 3 comprises a binding reagent for detecting 23403A (S protein D614), and Spot 8 comprises a binding reagent for detecting 23403G (S protein D614G mutation); Spot 4 comprises a binding reagent for detecting 23604C (S protein P681), and Spot 9 comprises a binding reagent for detecting 23604A (S protein P681H mutation); Spot 5 comprises a binding reagent for detecting 23664C (S protein A701), and Spot 10 comprises a binding reagent for detecting 23664T (S protein A701V mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein A69-70 deletion), and Spot 6 comprises a binding reagent for detecting the wild-type sequence at 21765-21770 (S protein residues 69-70); Spot 2 comprises a binding reagent for detecting 23063A (S protein N501), and Spot 7 comprises a binding reagent for detecting 23063T (S protein N501Y mutation); Spot 4 comprises a binding reagent for detecting 23604C (S protein P681), and Spot 9 comprises a binding reagent for detecting 23604A (S protein P681H mutation); Spot 7 comprises a binding reagent for detecting 22132G (S protein R190), and Spot 2 comprises a binding reagent for detecting 22132T (S protein R190S mutation); Spot 8 comprises a binding reagent for detecting 22206A (S protein D215), and Spot 3 comprises a binding reagent for detecting 22206G (S protein D215G mutation); Spot 9 comprises a binding reagent for detecting 22917T (S protein L452), and Spot 4 comprises a binding reagent for detecting 22917G (S protein L452R mutation). In embodiments, Spot 1 comprises a binding reagent for detecting 23012G (S protein E484), and Spot 6 comprises a binding reagent for detecting 23012A (S protein E484K mutation); Spot 5 comprises a binding reagent for detecting 23664C (S protein A701), and Spot 10 comprises a binding reagent for detecting 23664T (S protein A701V mutation), or Spot 5 comprises a binding reagent for detecting 22813G (S protein K417), and Spot 10 comprises a binding reagent for detecting 22813T (S protein K417N mutation); Spot 7 comprises a binding reagent for detecting 22812A (S protein K417), and Spot 2 comprises a binding reagent for detecting 22812C (S protein K417T mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein A69-70 deletion), and Spot 6 comprises a binding reagent for detecting the wild-type sequence at 21765-21770 (S protein residues 69-70); Spot 2 comprises a binding reagent for detecting 23063A (S protein N501), and Spot 7 comprises a binding reagent for detecting 23063T (S protein N501Y mutation); Spot 3 comprises a binding reagent for detecting 23403A (S protein D614), and Spot 8 comprises a binding reagent for detecting 23403G (S protein D614G mutation); Spot 4 comprises a binding reagent for detecting 23604C (S protein P681), and Spot 9 comprises a binding reagent for detecting 23604A (S protein P681H mutation); and Spot 5 comprises a binding reagent for detecting 22813G (S protein K417), and Spot 10 comprises a binding reagent for detecting 22813T (S protein K417N mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting 23012G (S protein E484), and Spot 6 comprises a binding reagent for detecting 23012A (S protein E484K mutation); Spot 7 comprises a binding reagent for detecting 22812A (S protein K417), and Spot 2 comprises a binding reagent for detecting 22812C (S protein K417T mutation); Spot 8 comprises a binding reagent for detecting 22206A (S protein D215), and Spot 3 comprises a binding reagent for detecting 22206G (S protein D215G mutation); Spot 9 comprises a binding reagent for detecting 22917T (S protein L452), and Spot 4 comprises a binding reagent for detecting 22917G (S protein L452R mutation); and Spot 10 comprises a binding reagent for detecting 22320A (S protein D253) and Spot 5 comprises a binding reagent for detecting 22320G (S protein D253G mutation).

In embodiments, the targeting probe for SARS-CoV-2 strain B.1.1.7 comprises any one of SEQ ID NOs:15, 16, 75, 76, 78, 79, 81, 82, 84, 85, 87, 88, 90, or 91. In embodiments, the detection probe for SARS-CoV-2 strain B.1.1.7 comprises any one of SEQ ID NOs:17, 77, 80, 83, 86, 89, or 92. In embodiments, the targeting probe for SARS-CoV-2 strain P.1 comprises any one of SEQ ID NOs:15, 16, 81, 82, 84, 85, 93, 94, 96, 97, 111, 112, 114, or 115. In embodiments, the detection probe for SARS-CoV-2 strain P.1 comprises any one of SEQ ID NOs:17, 83, 86, 95, 98, 113, or 116. In embodiments, the targeting probe for SARS-CoV-2 strain 501Y.V2 comprises any one of SEQ ID NOs:15, 16, 81, 82, 84, 85, 99, 100, 102, 103, 117, 118, 120, or 121. In embodiments, the detection probe for SARS-CoV-2 strain 501Y.V2 comprises any one of SEQ ID NOs: 17, 83, 86, 101, 104, 119, or 122. In embodiments, the targeting probe for SARS-CoV-2 strain Cal.20C comprises any one of SEQ ID NOs:105, 106, 108, or 109. In embodiments, the detection probe for SARS-CoV-2 strain Cal.20C comprises any one of SEQ ID NOs:107 or 110. SARS-CoV-2 strains are further described herein.

In embodiments, the targeting probe for SARS-CoV-2 location 21765-21770 comprises any one of SEQ ID NOs:75, 76, 78, or 79. In embodiments, the detection probe for SARS-CoV-2 location 21765-21770 comprises any one of SEQ ID NOs:77 or 80. In embodiments, the targeting probe for SARS-CoV-2 location 22132 comprises any one of SEQ ID NOs:93, 94, 96, or 97. In embodiments, the detection probe for SARS-CoV-2 location 22132 comprises any one of SEQ ID NOs:95 or 98. In embodiments, the targeting probe for SARS-CoV-2 location 22206 comprises any one of SEQ ID NOs:99, 100, 102, or 103. In embodiments, the detection probe for SARS-CoV-2 location 22206 comprises any one of SEQ ID NOs:101 or 104. In embodiments, the targeting probe for SARS-CoV-2 location 22917 comprises any one of SEQ ID NOs:105, 106, 108, or 109. In embodiments, the detection probe for SARS-CoV-2 location 22917 comprises any one of SEQ ID NOs:107 or 110. In embodiments, the targeting probe for SARS-CoV-2 location 23012 comprises any one of SEQ ID NOs:111, 112, 114, or 115. In embodiments, the detection probe for SARS-CoV-2 location 23012 comprises any one of SEQ ID NOs:113 or 116. In embodiments, the targeting probe for SARS-CoV-2 location 23063 comprises any one of SEQ ID NOs:81, 82, 84, or 85. In embodiments, the detection probe for SARS-CoV-2 location 23063 comprises any one of SEQ ID NOs:83 or 86. In embodiments, the targeting probe for SARS-CoV-2 location 23403 comprises SEQ ID NO:15 or 16. In embodiments, the detection probe for SARS-CoV-2 location 23403 comprises SEQ ID NO:17. In embodiments, the targeting probe for SARS-CoV-2 location 23604 comprises any one of SEQ ID NOs:87, 88, 90, or 91. In embodiments, the detection probe for SARS-CoV-2 location 23604 comprises any one of SEQ ID NO:89 or 92. In embodiments, the targeting probe for SARS-CoV-2 location 23664 comprises any one of SEQ ID NOs:117, 118, 120, or 121. In embodiments, the detection probe for SARS-CoV-2 location 23664 comprises any one of SEQ ID NOs:119 or 122.

In embodiments, the method provided herein is capable of detecting a viral nucleic acid or SNP therein using a CT cut-off value of about 25 to about 45, about 30 to about 40, about 31 to about 35, about 30, about 31, about 32, about 33, about 34, about 35, about 36, about 37, about 38, about 39, or about 40. Selection of CT cut-off values for nucleic acid detection is further described, e.g., in Caraguel et al., J Vet Diagn Invest 23:2-15 (2011).

Manual and Automated Embodiments

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477. In embodiments, the methods herein are performed in an automated cartridge reader as described herein.

In embodiments, automated systems, e.g., modular and fully integrated systems, for performing the methods herein comprises one or more of the following automated subsystems: a computer subsystem comprising hardware (e.g., personal computer, laptop, hardware processor, disc, keyboard, display, printer), software (e.g., processes such as drivers, driver controllers, and data analyzers), and/or a database; a liquid handling subsystem for sample and/or reagent handling, e.g., comprising a robotic pipetting hand, syringe, stirring apparatus, ultrasonic mixing apparatus, and/or magnetic mixing apparatus; a sample, reagent, and/or consumable storing and handling subsystem, e.g., comprising a robotic manipulator, tube or lid or foil piercing apparatus, lid removing apparatus, conveying apparatus such as linear or circular conveyor, tube rack, plate carrier, trough carrier, pipet tip carrier, plate shaker, and/or centrifuge; an assay reaction subsystem, e.g., that is fluid-based and/or consumable-based (such as tube and multi-well plate); a container and consumable washing subsystem. e.g., comprising a plate washing apparatus; a magnetic separator or magnetic particle concentrator subsystem, e.g., that is flow cell type, tube type, and/or plate type; a cell and particle detection, classification, and/or separation subsystem, e.g., comprising a flow cytometer and/or a Coulter counter; a detection subsystem, e.g., comprising a colorimetric detector, a nephelometric detector, a fluorescence detector, and/or an ECL detector; a temperature control subsystem, e.g., comprising an air handling system, air cooling system, air warming system, fan, blower, and/or water bath; a waste subsystem, e.g., comprising liquid and/or solid waste containers; a global unique identifier (GUI) detecting subsystem, e.g., comprising 1D and/or 2D barcode scanners such as flat bed and wand type scanners. In embodiments, the automated system further comprises a modular or fully integrated analytical subsystem, e.g., a chromatography system such as high-performance liquid chromatography (HPLC) or fast-protein liquid chromatography (FPLC), or a mass spectrometer.

In embodiments, systems or modules that perform sample identification and preparation are combined with, adjoined to, adjacent to, and/or robotically linked or coupled to the systems or modules that perform and/or detect the assays herein. Multiple modular systems of the same type can be combined to increase throughput. In embodiments, a modular system is combined with a module that performs other types of analysis, such as chemical, biochemical, and/or nucleic acid analysis.

In embodiments, the automated system allows batch, continuous, random-access, and/or point-of-care workflows, and single, medium, and high sample throughput.

In embodiments, the automated system comprises one or more of the following devices: a plate sealer (e.g., ZYMARK), a plate washer (e.g., BIOTEK, TECAN), a reagent dispenser, automated pipetting station, and/or liquid handling station (e.g., TECAN, ZYMARK, LABSYSTEMS, BECKMAN, HAMILTON), an incubator (e.g., ZYMARK), a plate shaker (e.g., Q.INSTRUMENTS, INHECO, THERMOFISHER SCIENTIFIC), a compound library module, a sample storage module, and/or a compound and/or sample retrieval module. In embodiments, one or more of these devices is coupled to the automated system via a robotic assembly such that the entire assay process can be performed automatically. In embodiments, a container (e.g., a plate) is manually moved between the apparatus and various devices described herein (e.g., a stack of plates).

In embodiments, the automated system is configured to perform one or more of the following functions: moving consumables such as plates into, within, and out of the detection subsystem; moving consumables between other subsystems; storing the consumables; sample and reagent handling (e.g., adapted to mix reagents and/or introduce reagents into consumables); consumable shaking (e.g., for mixing reagents and/or for increasing reaction rates); consumable washing (e.g., washing plates and/or performing assay wash steps (e.g., well aspirating)); measuring a detectable signal, e.g., ECL signal, in a flow cell or a consumable such as a tube or a plate. The automated system may be configured to handle individual tubes placed in racks and/or multi-well plates such as 96 or 384 well plates.

Methods for integrating components and modules in automated systems as described herein are discussed, e.g., by Sargeant et al., “Platform Perfection,” Medical Product Outsourcing, May 17, 2010.

In embodiments, the automated system is fully automated, modular, computerized, performs in vitro quantitative and qualitative tests on a wide range of analytes, and/or performs photometric assays, ion-selective electrode measurements, and/or electrochemiluminescence (ECL) assays. In embodiments, the system comprises one or more of the following hardware units: a control unit, a core unit and at least one analytical module.

In embodiments, the control unit utilizes a graphical user interface to control all instrument functions and comprises a readout device, such as a monitor; an input device, such as keyboard and mouse; and a personal computer, e.g., using a Windows operating system. In embodiments, the core unit comprises one or more components that manage conveyance of samples to each assigned analytical module. The actual composition of the core unit depends on the configuration of the analytical modules, which can be configured by one of skill in the art using methods known in the art. In embodiments, the core unit comprises at least the sampling unit and one rack rotor as main components. In embodiments, the control unit further comprises an extension unit, e.g., a conveyor line and/or a second rack rotor. In embodiments, the core unit further comprises a sample rack loader/unloader, a port, a barcode reader (for racks and samples), a water supply, and a system interface port. In embodiments, the automated system conducts ECL assays and comprises a reagent area, a measurement area, a consumables area, and a pre-clean area.

In embodiments, the methods herein are performed using an ultra high-throughput robotic liquid handling system. For example, the system can simultaneously prepare up to about 1600 samples with accuracy and reproducibility unmatched by a human operator. In embodiments, the system is a free-standing, fully integrated automated system as described herein. This system, capable of simultaneously running up to twenty 96-well assay plates, can include a robotic lab automation workstation for liquid handling and plate manipulation, and the workstation can be physically integrated with an ECL reader. In embodiments, the system comprises a liquid handling device (e.g., a pipettor), a barcode reader, a plate washing apparatus, a plate shaker, and a plate reader. In embodiments, the system conducts the methods described herein, e.g., the multiplexed immunoassays, with minimal human intervention. In embodiments, the ultra-high throughput system produces results for about 1600 samples in about 30 minutes to about 300 minutes, or about 60 minutes to about 150 minutes, or about 70 minutes to about 130 minutes. In embodiments, the ultra-high throughput system is capable of processing more than 1000 samples, more than 5000 samples, 10000 samples, more than 11000 samples, more than 12000 samples, more than 13000 samples, more than 14000 samples, or more than 15000 samples in a single day.

### Antibody and Composition

In embodiments, the invention provides an antibody or antigen-binding fragment thereof that specifically binds a viral antigen described herein, e.g., a SARS-CoV-2 protein. In embodiments, the invention provides an antibody or antigen-binding fragment thereof that specifically binds a SARS-CoV-2 N protein or a SARS-CoV-2 S protein. In embodiments, the invention provides an antibody or antigen-binding fragment thereof that specifically binds SARS-CoV-2 S1, S2, S-ECD, S-NTD, or S-RBD. In embodiments, the invention provides an antibody or antigen-binding fragment thereof that specifically binds a SARS-CoV-2 S protein or subunit or fragment thereof that comprises any of the mutations in Tables 1A and 1B. In embodiments, the invention provides an antibody or antigen-binding fragment thereof that specifically binds an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, an F protein from RSV, or a combination thereof.

In embodiments, the antibody or antigen-binding fragment thereof is a binding reagent, e.g., as disclosed herein, for an assay described herein, e.g., for detecting a viral component in a sample. In embodiments, the antibody or antigen-binding fragment thereof is capable of being immobilized onto a surface, e.g., as disclosed herein. In embodiments, the invention provides a composition comprising: (i) the antibody or antigen-binding fragment thereof; and (ii) a surface. In embodiments, the antibody or antigen-binding fragment thereof is immobilized onto the surface.

In embodiments, the antibody or antigen-binding fragment thereof is a detection reagent, e.g., as disclosed herein, for an assay described herein, e.g., for detecting a viral component in a sample. In embodiments, the antibody or antigen-binding fragment thereof comprises a detectable label. In embodiments, the antibody or antigen-binding fragment is capable of being conjugated with a detectable label. In embodiments, the invention provides a composition comprising: (i) the antibody or antigen-binding fragment thereof; (ii) a detectable label, e.g., as disclosed herein; and (iii) a reagent for conjugating the detectable label to the antibody or antigen-binding fragment thereof. In embodiments, the detectable label is an ECL label. In embodiments, the antibody or antigen-binding fragment thereof comprises a nucleic acid probe. In embodiments, the antibody or antigen-binding fragment is capable of being conjugated with a nucleic acid probe. In embodiments, the invention provides a composition comprising: (i) the antibody or antigen-binding fragment thereof; (ii) a nucleic acid probe, e.g., as disclosed herein; and (iii) a reagent for conjugating the nucleic acid probe to the antibody or antigen-binding fragment thereof.

In embodiments, the antibody or antigen-binding fragment thereof is a calibration reagent, e.g., as disclosed herein, for a serology assay, e.g., a classical, bridging, or competitive serology assay, e.g., as disclosed herein. In embodiments, the antibody or antigen-binding fragment thereof is a competitor for a competitive serology assay.

In embodiments, the invention provides a therapeutic composition comprising the antibody or antigen-binding fragment thereof. In embodiments, the therapeutic composition is capable of treating or preventing infection by a virus described herein, e.g., SARS-CoV-2 and/or a variant thereof.

In embodiments, the invention provides a composition comprising (i) the antibody or antigen-binding fragment thereof and (ii) a viral antigen that specifically binds the antibody or antigen-binding fragment.

In general, an antibody (used interchangeably with the term “immunoglobulin”) comprises at least the variable domain of a heavy chain; typically, an antibody comprises the variable domains of a heavy chain and a light chain. Both the heavy and light chains are divided into regions of structural and functional homology. Generally, the variable domain of a heavy chain (V_{H}) or light chain (V_{L}) determines antigen recognition and specificity, and the constant domain of a heavy chain (C_{H1}, C_{H2}, or C_{H3}) or light chain (C_{L}) confers biological properties such as secretion, receptor binding, complement binding, and the like. Generally, the N-terminal portion of an antibody chain is a variable portion, and the C-terminal portion is a constant region; the C_{H3} and C_{L} domains typically comprise the C-terminus of the heavy chain and light chain, respectively.

In general, antibodies are encoded by immunoglobulin genes or fragments of immunoglobulin genes. The recognized immunoglobulin genes include the kappa, lambda, alpha, gamma, delta, epsilon, and mu constant region genes, as well as immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

In general, the variable region allows the antibody to selectively recognize and specifically bind epitopes on antigens. Thus, the V_{L} domain and V_{H} domain, or a subset of the complementarity determining regions (CDR) within these variable domains, of an antibody combine to form the variable region that forms an antigen binding domain. The antigen binding domain is typically defined by three CDRs on each of the V_{L} and V_{H} domains. The six “complementarity determining regions” or “CDRs” typically present in each antigen binding domain are short, non-contiguous sequences of amino acids that are specifically positioned to form the antigen binding domain. The antigen binding domain formed by the positioned CDRs defines a surface complementary to the epitope on the antigen. This complementary surface promotes the non-covalent binding of the antibody to its cognate epitope.

In embodiments, the antibody or antigen-binding fragment thereof comprises a constant region comprising an IgA, IgD, IgE, IgG, or IgM domain. In embodiments, the antibody or antigen-binding fragment thereof comprises an IgG domain. In embodiments, the antibody or antigen-binding fragment thereof is an IgG1, IgG2, IgG3, or IgG4 isotype antibody or antigen-binding fragment thereof. In embodiments, the antibody or antigen-binding fragment thereof is IgG2a. IgG2b, or IgG2c subclass antibody or antigen-binding fragment thereof.

In embodiments, the antibody or antigen-binding fragment thereof is derived from a mouse, rat, goat, rabbit, chicken, guinea pig, hamster, horse, sheep, ferret, minx, or bat. In embodiments, the antibody or antigen-binding fragment thereof is humanized. In embodiments, the antibody or antigen-binding fragment thereof is capable of being administered to a human or an animal subject described herein, e.g., mouse, rat, ferret, minx, bat, a domestic animal, or an NHP. In embodiments, the antibody or antigen-binding fragment thereof is non-immunogenic to a human or an animal subject described herein, e.g., mouse, rat, ferret, minx, bat, a domestic animal, or an NHP.

### Kits

In embodiments, the invention provides a kit comprising, in one or more vials, containers, or compartments. (a) a binding reagent that specifically binds a respiratory virus component; and (b) a detection reagent that specifically binds the respiratory virus component. In embodiments, the kit further comprises a surface. Respiratory virus components and binding and detection reagents therefor are described herein. In embodiments, the binding reagent is an antibody or antigen-binding fragment. In embodiments, the detection reagent is an antibody or antigen-binding fragment thereof.

In embodiments, the respiratory virus component is a protein or fragment thereof. In embodiments, the respiratory virus is a coronavirus, an influenza virus, a paramyxovirus, an adenovirus, a bocavirus, a pneumovirus, an enterovirus, a rhinovirus, or a combination thereof. In embodiments, the respiratory virus is a coronavirus.

In embodiments, the invention provides a kit comprising, in one or more vials, containers, or compartments: (a) a binding reagent that specifically binds a coronavirus component; and (b) a detection reagent that specifically binds the coronavirus component. In embodiments, the kit further comprises a surface. Coronavirus components and binding and detection reagents therefor are described herein. In embodiments, the binding reagent is an antibody or antigen-binding fragment. In embodiments, the detection reagent is an antibody or antigen-binding fragment thereof.

In embodiments, the coronavirus component is a non-structural protein. In embodiments, the coronavirus is SARS-CoV, MERS-CoV, SARS-CoV-2, HCoV-OC43, HCoV-229E, HCoV-NL63, HCoV-HKU1, or a combination thereof.

In embodiments, the invention provides a kit comprising, in one or more vials, containers, or compartments: (a) a binding reagent that specifically binds a SARS-CoV-2 component; and (b) a detection reagent that specifically binds the SARS-CoV-2 component. In embodiments, the kit further comprises a surface. In embodiments, the binding reagent is an antibody or antigen-binding fragment. In embodiments, the binding reagent comprises biotin, and the surface comprises avidin or streptavidin. In embodiments, the detection reagent is an antibody or antigen-binding fragment thereof. In embodiments, the detection reagent comprises a detectable label, e.g., an ECL label as described herein. In embodiments, the SARS-CoV-2 component comprises an N protein, an S protein, or both. In embodiments, the SARS-CoV-2 component comprises an N protein, an S-RBD, or both.

In embodiments, the invention provides a kit comprising, in one or more vials, containers, or compartments: (a) a viral antigen that specifically binds a biomarker, e.g., an antibody biomarker; and (b) a detection reagent that specifically binds the biomarker, e.g., the antibody biomarker. In embodiments, the kit further comprises a surface. Antibody biomarkers and their binding partners, e.g., viral antigens, are described herein. In embodiments, the detection reagent is an antibody or antigen-binding fragment thereof. In embodiments, the detection reagent is a second copy of the viral antigen.

In embodiments, the viral antigen is a respiratory virus antigen. In embodiments, the respiratory virus is a coronavirus, an influenza virus, a paramyxovirus, an adenovirus, a bocavirus, a pneumovirus, an enterovirus, a rhinovirus, or a combination thereof. In embodiments, the viral antigen is a coronavirus S protein or fragment thereof. In embodiments, the coronavirus is SARS-CoV, MERS-CoV, SARS-CoV-2. HCoV-OC43, HCoV-229E, HCoV-NL63, HCoV-HKU1, or a combination thereof. In embodiments, the viral antigen is SARS-CoV-2 S protein, S1 subunit, S2 subunit, S-RBD, M protein, E protein, N protein, or a combination thereof.

In embodiments, the invention provides a kit comprising, in one or more vials, containers, or compartments: (a) a binding reagent that specifically binds a biomarker, e.g., an inflammatory or tissue damage response biomarker; and (b) a detection reagent that specifically binds the biomarker, e.g., the inflammatory or tissue damage response biomarker. In embodiments, the kit further comprises a surface. Inflammatory and tissue damage response biomarkers and binding and detection reagents therefor are described herein. In embodiments, the binding reagent is an antibody or antigen-binding fragment. In embodiments, the detection reagent is an antibody or antigen-binding fragment thereof. In embodiments, the biomarker is G-CSF. In embodiments, the biomarker is GM-CSF. In embodiments, the biomarker is IFN-α2a. In embodiments, the biomarker is IL-4. In embodiments, the biomarker is IL-6. In embodiments, the biomarker is IL-10. In embodiments, the biomarker is TNF-α. In embodiments, the biomarker is ferritin.

In embodiments, the kit comprises multiple binding reagents and multiple detection reagents, wherein each binding reagent or detection reagent specifically binds to one of IL-1D, IL-6, IL-8, or TNF-α. In embodiments, the kit comprises multiple binding reagents and multiple detection reagents, wherein each binding reagent or detection reagent specifically binds to one of IFN-γ, IL-1β, IL-4, IL-6, IL-8, IL-10, or TNF-α. In embodiments, the kit comprises multiple binding reagents and multiple detection reagents, wherein each binding reagent or detection reagent specifically binds to one of IFN-γ, IL-1β, IL-4, IL-5, IL-6, IL-8, IL-10, IL-12p70, or TNF-α. In embodiments, the kit comprises multiple binding reagents and multiple detection reagents, wherein each binding reagent or detection reagent specifically binds to one of IFN-γ, IL-1β, IL-2, IL-4, IL-6, IL-8, IL-10, IL-12p70, IL-13, or TNF-α.

In embodiments, the kit comprises multiple binding reagents and multiple detection reagents, wherein each binding reagent or detection reagent specifically binds to one of CRP, ICAM-1, SAA, or VCAM-1. In embodiments, the kit comprises multiple binding reagents and multiple detection reagents, wherein each binding reagent or detection reagent specifically binds to one of CRP, LBP, ICAM-1, SAA, or VCAM-1. In embodiments, the kit comprises multiple binding reagents and multiple detection reagents, wherein each binding reagent or detection reagent specifically binds to one of CRP, Eotaxin, Eotaxin-3, FGF (basic), VEGFR-1/Flt-1, GM-CSF, ICAM-1, IFN-γ, IL-1α, IL-1β, IL-2, IL-3, IL-4, IL-5, IL-6, IL-7, IL-8, IL-8 (HA), IL-9, IL-10, IL-12/IL-23p40, IL-12p70, IL-13, IL-15, IL-16, IL-17A, IL-17A/F, IL-17B, IL-17C, IL-17D, IL-1RA, IL-21, IL-22, IL-23, IL-27, IL-31, IP-10, MCP-1, MCP-4, MDC, MIP-1α, MIP-1β, MIP-3α, PlGF, SAA, TARC, Tie-2, TNF-α, TNF-β, TSLP, VCAM-1, VEGF-A, VEGF-C, or VEGF-D.

In embodiments, the kit comprises multiple binding reagents and multiple detection reagents, wherein each binding reagent or detection reagent specifically binds to one of IL-1β, IL-6, or IL-8. In embodiments, the kit comprises multiple binding reagents and multiple detection reagents, wherein each binding reagent or detection reagent specifically binds to one of IFN-γ, IL-1β, IL-6, IL-8, or IL-10. In embodiments, the kit comprises multiple binding reagents and multiple detection reagents, wherein each binding reagent or detection reagent specifically binds to one of IFN-γ, IL-1β, IL-5, IL-6, IL-8, or IL-10. In embodiments, the kit comprises multiple binding reagents and multiple detection reagents, wherein each binding reagent or detection reagent specifically binds to one of IFN-γ, IL-1β, IL-2, IL-6, IL-8, or IL-10.

In embodiments, the kit comprises multiple binding reagents and multiple detection reagents, wherein each binding reagent or detection reagent specifically binds to one of G-CSF, GM-CSF, IFN-α2a, IFN-β, IFN-γ, IL-1RA, IL-1β, IL-4, IL-5, IL-6, IL-7, IL-8, IL-9, IL-10, IL-12p70, IP-10, MCP1, MIP-1α, TNF-α, or VEGF-A. In embodiments, the kit comprises multiple binding reagents and multiple detection reagents, wherein each binding reagent or detection reagent specifically binds to one of GM-CSF, IL-1α, IL-5, IL-7, IL-12/IL-23p40, IL-15, IL-16, IL-17A, TNF-β, or VEGF-A.

In embodiments, the kit comprises multiple binding reagents and multiple detection reagents, wherein each binding reagent or detection reagent specifically binds to one of G-CSF, GM-CSF, IFN-α2a, IFN-γ, IL-1RA, IL-10, IL-4, IL-6, IL-7, IL-8, IL-9, IL-10, IL-12p70, IP-10, MCP1, MIP-1α, TNF-α, or VEGF-A. In embodiments, the kit comprises multiple binding reagents and multiple detection reagents, wherein each binding reagent or detection reagent specifically binds to one of GM-CSF, IL-1α, IL-5, IL-7, IL-12/IL-23p40, IL-15, IL-16, IL-17A, TNF-β, or VEGF-A.

In embodiments, the kit comprises multiple binding reagents and multiple detection reagents, wherein each binding reagent or detection reagent specifically binds to one of IFN-γ, IL-1β, IL-2, IL-4, IL-6, IL-10, IL-12p70, IL-17A, and TNF-α in a biological sample. In embodiments, the kit comprises multiple binding reagents and multiple detection reagents, wherein each binding reagent or detection reagent specifically binds to one of GFAP, Tau, and NF-L in a biological sample.

In embodiments, the kit comprises multiple binding reagents and multiple detection reagents, wherein each binding reagent or detection reagent specifically binds to one of CD27, CD28, CD40L (soluble), CTLA-4, GITR/TNFRSF18, HAVCR2/TIM-3, LAG3, OX40, PD1, and TIGIT. In embodiments, the kit comprises multiple binding reagents and multiple detection reagents, wherein each binding reagent or detection reagent specifically binds to one of CD27, CD28, CD40L (soluble), CTLA-4, GITR/TNFRSF18, HAVCR2/TIM-3, LAG3, OX40, PD1, TIGIT, Tie-2, and gp130.

In embodiments, the invention provides a kit comprising. (a) one or more binding reagents that specifically binds one or more antibody biomarkers, wherein each antibody biomarker specifically binds to one or more of the S protein, S1 subunit, S2 subunit, S-RBD, S-NTD, N protein, E protein, or M protein from SARS-CoV-2; and (b) one or more detection reagent that specifically binds the one or more antibody biomarkers. In embodiments, the one or more binding reagents comprises the S protein, S1 subunit, S2 subunit, S-RBD, S-NTD, N protein, E protein, and M protein from SARS-CoV-2. In embodiments, the kit further comprises a surface.

In embodiments, the invention provides a kit comprising: (a) one or more binding reagents that specifically bind one or more antibody biomarkers, wherein each antibody biomarker specifically binds to one or more of the S protein from HCoV-HKU1, S protein from HCoV-NL63, S protein from HCoV-229E, S protein from HCoV-OC43, S protein from SARS-CoV, or S protein from MERS-CoV; and (b) one or more detection reagent that specifically binds the one or more antibody biomarkers. In embodiments, the one or more binding reagents comprises the S protein from HCoV-HKU1, S protein from HCoV-NL63, S protein from HCoV-229E, S protein from HCoV-OC43, S protein from SARS-CoV, and S protein from MERS-CoV. In embodiments, the kit further comprises a surface.

In embodiments, the invention provides a kit comprising: (a) one or more binding reagents that specifically bind one or more antibody biomarkers, wherein each antibody biomarker specifically binds to one or more of the S protein from SARS-CoV-2, S1 subunit from SARS-CoV-2, S2 subunit from SARS-CoV-2, S-RBD from SARS-CoV-2, S-NTD from SARS-CoV-2, N protein from SARS-CoV-2, E protein from SARS-CoV-2, M protein from SARS-CoV-2, S protein from HCoV-HKU1, S protein from HCoV-NL63, S protein from HCoV-229E, S protein from HCoV-OC43, S protein from SARS-CoV, or S protein from MERS-CoV, or combination thereof; and (b) one or more detection reagent that specifically binds the one or more antibody biomarkers. In embodiments, the one or more binding reagents comprises the S protein from SARS-CoV-2, S1 subunit from SARS-CoV-2, S2 subunit from SARS-CoV-2, S-RBD from SARS-CoV-2, S-NTD from SARS-CoV-2, N protein from SARS-CoV-2, E protein from SARS-CoV-2, M protein from SARS-CoV-2. S protein from HCoV-HKU1, S protein from HCoV-NL63, S protein from HCoV-229E, S protein from HCoV-OC43, S protein from SARS-CoV, and S protein from MERS-CoV. In embodiments, the kit further comprises a surface.

In embodiments, the invention provides a kit comprising: (a) one or more binding reagents that specifically bind one or more antibody biomarkers, wherein each antibody biomarker specifically binds to one or more of the HA protein from influenza B, HA protein from influenza A H1, HA protein from influenza A H3, HA protein from influenza A H7, or F protein from RSV; and (b) one or more detection reagent that specifically binds the one or more antibody biomarkers. In embodiments, the one or more binding reagents comprises the HA protein from influenza B, HA protein from influenza A H1, HA protein from influenza A H3, HA protein from influenza A H7, and F protein from RSV. In embodiments, the kit further comprises a surface.

In embodiments, the invention provides a kit comprising: (a) one or more binding reagents that specifically bind one or more antibody biomarkers, wherein each antibody biomarker specifically binds to: one or more of the S protein from SARS-CoV-2, S1 subunit from SARS-CoV-2, S2 subunit from SARS-CoV-2, S-RBD from SARS-CoV-2, S-NTD from SARS-CoV-2, N protein from SARS-CoV-2, E protein from SARS-CoV-2. M protein from SARS-CoV-2, HA protein from influenza B, HA protein from influenza A H1, HA protein from influenza A H3, HA protein from influenza A H7, or F protein from RSV; and (b) one or more detection reagent that specifically binds the one or more antibody biomarkers. In embodiments, the one or more binding reagents comprises the S protein from SARS-CoV-2, S1 subunit from SARS-CoV-2, S2 subunit from SARS-CoV-2, S-RBD from SARS-CoV-2, S-NTD from SARS-CoV-2, N protein from SARS-CoV-2, E protein from SARS-CoV-2, M protein from SARS-CoV-2, HA protein from influenza B, HA protein from influenza A H1, HA protein from influenza A H3, HA protein from influenza A H7, and F protein from RSV. In embodiments, the kit further comprises a surface.

In embodiments, the invention provides a kit comprising: (a) one or more binding reagents that specifically binds one or more antibody biomarkers, wherein each antibody biomarker specifically binds to one or more of an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, and an F protein from RSV; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the S protein from SARS-CoV-2, S protein from SARS-CoV, S protein from MERS-CoV, S protein from HCoV-HKU1, S protein from HCoV-OC43, S protein from HCoV-NL63, S protein from HCoV-229E. N protein from SARS-CoV-2, N protein from SARS-CoV, N protein from MERS-CoV, N protein from HCoV-HKU1, N protein from HCoV-OC43, N protein from HCoV-NL63, N protein from HCoV-229E, HA from influenza B, HA from influenza A H1, HA from influenza A H3, HA from influenza A H7, and F protein from RSV. In embodiments, the one or more detection reagents specifically binds to the antibody biomarker. In embodiments, the one or more detection reagents specifically binds to IgG. IgA, or IgM. In embodiments, the one or more detection reagents comprises the S protein from SARS-CoV-2, S protein from SARS-CoV. S protein from MERS-CoV, S protein from HCoV-HKU1, S protein from HCoV-OC43, S protein from HCoV-NL63, S protein from HCoV-229E. N protein from SARS-CoV-2, N protein from SARS-CoV, N protein from MERS-CoV, N protein from HCoV-HKU1, N protein from HCoV-OC43, N protein from HCoV-NL63, N protein from HCoV-229E. HA from influenza B, HA from influenza A H1, HA from influenza A H3, HA from influenza A H7, and F protein from RSV. In embodiments, the one or more detection reagents comprises ACE2. In embodiments, the kit further comprises a surface.

In embodiments, the invention provides a kit comprising: (a) one or more binding reagents that specifically binds one or more antibody biomarkers, wherein each antibody biomarker specifically binds to one or more of an N protein from SARS-CoV-2, an S protein from SARS-CoV-2, an S-RBD from SARS-CoV-2, an S-NTD from SARS-CoV-2, an S protein from SARS-CoV, an S-protein from MERS-CoV, an S protein from HCoV-HKU1, an S protein from HcoV-OC43, an HA from influenza strain B/Brisbane, an HA from influenza strain B/Phuket, an HA from influenza strain H1/Michigan, an HA from influenza strain H3/Hong Kong, and an HA from influenza strain H7/Shanghai; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S protein from SARS-CoV-2, S-RBD from SARS-CoV-2, S-NTD from SARS-CoV-2. S protein from SARS-CoV, S-protein from MERS-CoV, S protein from HCoV-HKU1, S protein from HcoV-OC43, HA from influenza strain B/Brisbane, HA from influenza strain B/Phuket, HA from influenza strain H1/Michigan, HA from influenza strain H3/Hong Kong, and HA from influenza strain H7/Shanghai. In embodiments, the one or more detection reagents specifically binds to the antibody biomarker. In embodiments, the one or more detection reagents specifically binds to IgG, IgA, or IgM. In embodiments, the one or more detection reagents comprises the N protein from SARS-CoV-2, S protein from SARS-CoV-2, S-RBD from SARS-CoV-2, S-NTD from SARS-CoV-2, S protein from SARS-CoV, S-protein from MERS-CoV, S protein from HCoV-HKU1, S protein from HcoV-OC43, HA from influenza strain B/Brisbane, HA from influenza strain B/Phuket, HA from influenza strain H1/Michigan, HA from influenza strain H3/Hong Kong, and HA from influenza strain H7/Shanghai. In embodiments, the one or more detection reagents comprises ACE2. In embodiments, the kit further comprises a surface.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2. S protein from SARS-CoV-2, or S protein from SARS-CoV; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2, S-RBD from SARS-CoV-2, S protein from SARS-CoV-2, and S protein from SARS-CoV. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2, S-RBD from SARS-CoV-2, S protein from SARS-CoV-2, and S protein from SARS-CoV. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 3 of FIG. 39B comprises an immobilized SARS-CoV-2 N protein, Spot 4 of FIG. 39B comprises an immobilized SARS-CoV S protein, Spot 8 of FIG. 39B comprises an immobilized SARS-CoV-2 S-NTD protein, Spot 10 of FIG. 39B comprises an immobilized SARS-CoV-2 S-RBD, and Spots 2, 5, 6, 7, and 9 of FIG. 39B each comprises an immobilized BSA.

In embodiments, the invention provides a kit comprising. (a) one or more binding reagents that specifically bind one or more antibody biomarkers, wherein each antibody biomarker specifically binds to: one or more of the N protein from SARS-CoV-2, S-RBD from SARS-CoV-2, or S protein from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-RBD from SARS-CoV-2, and S protein from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises the N protein from SARS-CoV-2. S-RBD from SARS-CoV-2, and S protein from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, w % herein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 3 of FIG. 39B comprises an immobilized SARS-CoV-2 N protein, Spot 10 of FIG. 39B comprises an immobilized SARS-CoV-2 S-RBD, and Spots 2, 4, 5, 6, 7, 8, and 9 of FIG. 39B each comprises an immobilized BSA. In embodiments, the surface comprises a multi-well assay plate, wherein each well comprises four distinct binding domains. In embodiments, the assay plate is a 384-well plate. An embodiment of a well in a 384-well assay plate, comprising four binding domains (“spots”) is shown in FIG. 39A. In embodiments, Spot A1 of FIG. 39A comprises an immobilized SARS-CoV-2 S protein. Spot A2 of FIG. 39A comprises an immobilized SARS-CoV-2 N protein, Spot B1 of FIG. 39A comprises an immobilized SARS-CoV-2 S-RBD, and Spot B2 of FIG. 39A comprises an immobilized BSA.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2; and (b) one or more detection reagents. In embodiments, the one or more antibody biomarkers comprises IgG, IgA, IgM, or a combination thereof. In embodiments, the IgG. IgA, and/or IgM is from a human, mouse, rat, ferret, minx, bat, or combination thereof. In embodiments, the one or more binding reagents comprises a wild-type S protein from SARS-CoV-2, an Orf18 oligomer from SARS-CoV-2, an N protein from SARS-CoV-2, a Mem protein from SARS-CoV-2, an Orf7a protein from SARS-CoV-2, an Env protein from SARS-CoV-2, an Orf8 (2) protein from SARS-CoV-2, and an S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an Orf8 monomer from SARS-CoV-2, an N protein from SARS-CoV-2, a Mem protein from SARS-CoV-2, an Orf7a protein from SARS-CoV-2, an Env protein from SARS-CoV-2, an Orf8 (2) protein from SARS-CoV-2, and an S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized an Orf8 oligomer from SARS-CoV-2. Spot 3 of FIG. 39B comprises an N protein from immobilized SARS-CoV-2, Spot 4 of FIG. 39B comprises an immobilized a Mem protein from SARS-CoV-2, Spot 6 of FIG. 39B comprises an immobilized an Orf7a protein from SARS-CoV-2, Spot 7 of FIG. 39B comprises an Env protein from SARS-CoV-2, Spot 9 of FIG. 39B comprises an Orf8 monomer from SARS-CoV-2, Spot 10 of FIG. 39B comprises a wild-type S-RBD from SARS-CoV-2, Spot 5 of FIG. 39B comprises an immobilized BSA, and Spot 8 of FIG. 39B comprises an immobilized human serum albumin (HSA).

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2, and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2. Spot 3 of FIG. 39B comprises an N protein from immobilized SARS-CoV-2. Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, and Spots 2, 4, 5, 6, and 10 of FIG. 39B each comprises an immobilized BSA.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an S-D614G from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2. Spot 10 of FIG. 39B comprises a wild-type S-RBD from SARS-CoV-2, and Spots 4, 5, and 6 of FIG. 39B each comprises an immobilized BSA.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an S-D614G from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, and Spots 4, 5, 6, and 10 of FIG. 39B each comprises an immobilized BSA.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents specifically binds to the antibody biomarker. In embodiments, the one or more detection reagents specifically binds IgA. IgG, or IgM. In embodiments, the one or more detection reagents comprises a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an S-RBD from SARS-CoV-2 strain 501Y.V2, Spot 3 of FIG. 39B comprises an N protein from immobilized SARS-CoV-2, Spot 4 of FIG. 39B comprises an S-RBD from SARS-CoV-2 strain P.1, Spot 6 of FIG. 39B comprises an S-RBD from SARS-CoV-2 strain B.1.1.7, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spot 5 of FIG. 39B each comprises an immobilized BSA.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2. Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA.

In embodiments, the invention provides a kit comprising. (a) one or more binding reagents that specifically bind one or more antibody biomarkers, wherein each antibody biomarker specifically binds to: one or more of the N protein from SARS-CoV-2, S-NTD from SARS-CoV-2. S-RBD from SARS-CoV-2, S protein from SARS-CoV-2, S protein from SARS-CoV, S protein from MERS-CoV, S protein from HCoV-OC43, S protein from HCoV-HKU1, or HA protein from influenza A H3; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2, S-RBD from SARS-CoV-2, S protein from SARS-CoV-2, S protein from SARS-CoV, S protein from MERS-CoV, S protein from HCoV-OC43, S protein from HCoV-HKU1, and HA protein from influenza A/Hong Kong H3. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2, S-RBD from SARS-CoV-2. S protein from SARS-CoV-2, S protein from SARS-CoV, S protein from MERS-CoV, S protein from HCoV-OC43, S protein from HCoV-HKU1, and HA protein from influenza A H3. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized influenza A/Hong Kong H3 HA protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized MERS-CoV S protein, Spot 10 of FIG. 39B comprises an immobilized SARS-CoV-2 S-RBD, and Spot 5 of FIG. 39B comprises an immobilized BSA.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2, S protein from SARS-CoV-2, S protein from SARS-CoV. S protein from MERS-CoV, S protein from HCoV-OC43, S protein from HCoV-HKU1, HA protein from influenza A H3, HA protein from influenza A H1; HA protein from influenza A H7, HA protein from influenza B/Phu, or HA protein from influenza B/Bris; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2, S-RBD from SARS-CoV-2, S protein from SARS-CoV-2. S protein from SARS-CoV, S protein from MERS-CoV. S protein from HCoV-OC43, S protein from HCoV-HKU1, HA protein from influenza A H3, HA protein from influenza A H1; HA protein from influenza A H7, HA protein from influenza B/Phu, and HA protein from influenza B/Bris. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2, S-RBD from SARS-CoV-2. S protein from SARS-CoV-2, S protein from SARS-CoV, S protein from MERS-CoV, S protein from HCoV-OC43, S protein from HCoV-HKU1, HA protein from influenza A H3, HA protein from influenza A H1; HA protein from influenza A H7, HA protein from influenza B/Phu, and HA protein from influenza B/Bris. In embodiments, the one or more detection reagents comprises ACE2. In embodiments, the kit further comprises a surface.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2. S protein from SARS-CoV-2, S protein from SARS-CoV, S protein from MERS-CoV, S protein from HCoV-OC43, S protein from HCoV-HKU1, HA protein from influenza A H3, HA protein from influenza A H1; HA protein from influenza A H7, HA protein from influenza B/Phu, HA protein from influenza B/Bris, or F protein from RSV; and (b) one or more detection reagents. In embodiments, the one or more antibody biomarkers comprises IgG, IgA, IgM, or a combination thereof. In embodiments, the IgG. IgA, and/or IgM is from a human, mouse, rat, ferret, minx, bat, or combination thereof. In embodiments, the one or more binding reagents comprises the N protein from SARS-CoV-2. S-NTD from SARS-CoV-2, S-RBD from SARS-CoV-2, S protein from SARS-CoV-2, S protein from SARS-CoV. S protein from MERS-CoV, S protein from HCoV-OC43, S protein from HCoV-HKU1, HA protein from influenza A/Hong Kong H3, HA protein from influenza A/Michigan H1; HA protein from influenza A/Shanghai H7, HA protein from influenza B/Phu, HA protein from influenza B/Bris, and F protein from RSV. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2. S-RBD from SARS-CoV-2, S protein from SARS-CoV-2, S protein from SARS-CoV, S protein from MERS-CoV. S protein from HCoV-OC43, S protein from HCoV-HKU1, HA protein from influenza A H3, HA protein from influenza A H1; HA protein from influenza A H7, HA protein from influenza B/Phu, HA protein from influenza B/Bris, and F protein from RSV. In embodiments, the one or more detection reagents comprises ACE2. In embodiments, the kit further comprises a surface. In embodiments, the surface comprises two assay plates, wherein each assay plate is a 96-well plate.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2, S protein from SARS-CoV-2, S protein from SARS-CoV. S protein from HCoV-OC43, S protein from HCoV-HKU1, S protein from HCoV-NL63, or S protein from HCoV-229E; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2, S-RBD from SARS-CoV-2, S protein from SARS-CoV-2, S protein from SARS-CoV. S protein from HCoV-OC43. S protein from HCoV-HKU1, S protein from HCoV-NL63, and S protein from HCoV-229E. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2, S-RBD from SARS-CoV-2, S protein from SARS-CoV-2. S protein from SARS-CoV, S protein from HCoV-OC43, S protein from HCoV-HKU1, S protein from HCoV-NL63, and S protein from HCoV-229E. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized HCoV-NL63 S protein, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S-NTD, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, the invention provides a kit comprising: (a) one or more binding reagents that specifically bind one or more antibody biomarkers, wherein each antibody biomarker specifically binds to: one or more of the N protein from SARS-CoV-2, S-RBD from SARS-CoV-2, S protein from SARS-CoV-2. S protein from SARS-CoV, S protein from MERS-CoV, S protein from HCoV-OC43, S protein from HCoV-HKU1, S protein from HCoV-NL63, or S protein from HCoV-229E; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-RBD from SARS-CoV-2, S protein from SARS-CoV-2, S protein from SARS-CoV, S protein from MERS-CoV, S protein from HCoV-OC43, S protein from HCoV-HKU1, S protein from HCoV-NL63, and S protein from HCoV-229E. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-RBD from SARS-CoV-2, S protein from SARS-CoV-2, S protein from SARS-CoV. S protein from MERS-CoV, S protein from HCoV-OC43, S protein from HCoV-HKU1, S protein from HCoV-NL63, and S protein from HCoV-229E. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments. Spot 1 of FIG. 39B comprises an immobilized SARS-CoV-2 S protein, Spot 2 of FIG. 39B comprises an immobilized S protein from HCoV-NL63, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized MERS-CoV S protein, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, the invention provides a kit comprising. (a) one or more binding reagents that specifically bind one or more antibody biomarkers, wherein each antibody biomarker specifically binds to: one or more of the N protein from SARS-CoV-2, S-RBD from SARS-CoV-2. S protein from SARS-CoV-2, S protein from SARS-CoV, S2 from SARS-CoV-2, S protein from HCoV-OC43, S protein from HCoV-HKU1, S protein from HCoV-NL63, or S protein from HCoV-229E; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-RBD from SARS-CoV-2, S protein from SARS-CoV-2, S protein from SARS-CoV, S2 from SARS-CoV-2, S protein from HCoV-OC43, S protein from HCoV-HKU1, S protein from HCoV-NL63, and S protein from HCoV-229E. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-RBD from SARS-CoV-2, S protein from SARS-CoV-2. S protein from SARS-CoV, S2 from SARS-CoV-2, S protein from HCoV-OC43, S protein from HCoV-HKU1, S protein from HCoV-NL63, and S protein from HCoV-229E. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S protein, Spot 2 of FIG. 39B comprises an immobilized S protein from HCoV-NL63, Spot 3 of FIG. 39B comprises an immobilized SARS-CoV-2 N protein, Spot 4 of FIG. 39B comprises an immobilized SARS-CoV S protein, Spot 6 of FIG. 39B comprises an immobilized SARS-CoV-2 S2, Spot 7 of FIG. 39B comprises an immobilized HCoV-HKU1 S protein, Spot 8 of FIG. 39B comprises an immobilized HCoV-OC43 S protein, Spot 9 of FIG. 39B comprises an immobilized HCoV-229E S protein, Spot 10 of FIG. 39B comprises an immobilized SARS-CoV-2 S-RBD, and Spot 5 of FIG. 39B comprises an immobilized BSA.

In embodiments, the invention provides a kit comprising: (a) one or more binding reagents that specifically bind one or more antibody biomarkers, wherein each antibody biomarker specifically binds to: one or more of an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2. In embodiments, the one or more detection reagents specifically binds to the antibody biomarker. In embodiments, the detection reagent specifically binds IgA, IgG, or IgM. In embodiments, the one or more detection reagents comprises an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized S protein from SARS-CoV-2. Spot 2 of FIG. 39B comprises an immobilized an S1 from HCoV-NL63, Spot 3 of FIG. 39B comprises an immobilized N protein from SARS-CoV-2, Spot 4 of FIG. 39B comprises an immobilized S1 from SARS-CoV, Spot 6 of FIG. 39B comprises an immobilized S1 from SARS-CoV-2, Spot 7 of FIG. 39B comprises an immobilized S1 from HCoV-HKU1, Spot 8 of FIG. 39B comprises an immobilized S1 from HCoV-OC43. Spot 9 of FIG. 39B comprises an immobilized S1 from HCoV-229E, Spot 10 of FIG. 39B comprises an immobilized S-RBD from SARS-CoV-2, and Spot 5 of FIG. 39B comprises an immobilized BSA.

In embodiments, the invention provides a kit comprising: (a) one or more binding reagents that specifically bind one or more antibody biomarkers, wherein each antibody biomarker specifically binds to: one or more of an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2. In embodiments, the one or more detection reagents specifically binds to the antibody biomarker. In embodiments, the detection reagent specifically binds IgA, IgG, or IgM. In embodiments, the one or more detection reagents comprises an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments. Spot 1 of FIG. 39B comprises an immobilized SARS-CoV-2 S protein, Spot 2 of FIG. 39B comprises an immobilized an N protein from HCoV-NL63, Spot 3 of FIG. 39B comprises an immobilized N protein from SARS-CoV-2, Spot 4 of FIG. 39B comprises an immobilized N protein from SARS-CoV, Spot 6 of FIG. 39B comprises an immobilized N protein from MERS-CoV, Spot 7 of FIG. 39B comprises an immobilized N protein from HCoV-HKU1, Spot 8 of FIG. 39B comprises an immobilized N protein from HCoV-OC43, Spot 9 of FIG. 39B comprises an N protein from HCoV-229E, Spot 10 of FIG. 39B comprises an immobilized S-RBD from SARS-CoV-2, and Spot 5 of FIG. 39B comprises an immobilized BSA.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2, S protein from SARS-CoV-2, S protein from SARS-CoV, S protein from HCoV-OC43, S protein from HCoV-HKU1, S protein from HCoV-NL63, S protein from HCoV-229E, HA protein from influenza A H3, HA protein from influenza A H1; HA protein from influenza A H7, HA protein from influenza B/Phu. HA protein from influenza B/Bris, or F protein from RSV; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2, S-RBD from SARS-CoV-2, S protein from SARS-CoV-2, S protein from SARS-CoV. S protein from HCoV-OC43. S protein from HCoV-HKU1, S protein from HCoV-NL63, S protein from HCoV-229E, HA protein from influenza A/Hong Kong H3, HA protein from influenza A/Michigan H1; HA protein from influenza A/Shanghai H7, HA protein from influenza B/Phu, HA protein from influenza B/Bris, and F protein from RSV. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2. S-RBD from SARS-CoV-2, S protein from SARS-CoV-2, S protein from SARS-CoV, S protein from HCoV-OC43, S protein from HCoV-HKU1, S protein from HCoV-NL63, S protein from HCoV-229E, HA protein from influenza A H3, HA protein from influenza A H1; HA protein from influenza A H7, HA protein from influenza B/Phu, HA protein from influenza B/Bris, and F protein from RSV. In embodiments, the one or more detection reagents comprises ACE2. In embodiments, the kit further comprises a surface. In embodiments, the surface comprises two assay plates, wherein each assay plate is a 96-well plate.

In embodiments, the invention provides a kit comprising. (a) one or more binding reagents that specifically bind one or more antibody biomarkers, wherein each antibody biomarker specifically binds to: one or more of the N protein from SARS-CoV-2, S-RBD from SARS-CoV-2. S protein from SARS-CoV-2, S protein from SARS-CoV, S protein from MERS-CoV, S protein from HCoV-OC43, S protein from HCoV-HKU1, S protein from HCoV-NL63, S protein from HCoV-229E, HA protein from influenza A H3, HA protein from influenza A H1; HA protein from influenza A H7. HA protein from influenza B/Phu. HA protein from influenza B/Bris, or F protein from RSV; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2. S-RBD from SARS-CoV-2, S protein from SARS-CoV-2, S protein from SARS-CoV, S protein from MERS-CoV, S protein from HCoV-OC43, S protein from HCoV-HKU1, S protein from HCoV-NL63, S protein from HCoV-229E, HA protein from influenza A/Hong Kong H3, HA protein from influenza A/Michigan H1; HA protein from influenza A/Shanghai H7, HA protein from influenza B/Phu, HA protein from influenza B/Bris, and F protein from RSV. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-RBD from SARS-CoV-2, S protein from SARS-CoV-2, S protein from SARS-CoV, S protein from MERS-CoV, S protein from HCoV-OC43. S protein from HCoV-HKU1, S protein from HCoV-NL63, S protein from HCoV-229E, HA protein from influenza A H3, HA protein from influenza A H1; HA protein from influenza A H7, HA protein from influenza B/Phu, HA protein from influenza B/Bris, and F protein from RSV. In embodiments, the one or more detection reagents comprises ACE2. In embodiments, the kit further comprises a surface. In embodiments, the surface comprises two assay plates, wherein each assay plate is a 96-well plate.

In embodiments, the invention provides a kit comprising: (a) one or more binding reagents that specifically bind one or more antibody biomarkers, wherein each antibody biomarker specifically binds to: one or more of the S protein from SARS-CoV-2, S-RBD from SARS-CoV-2, S-NTD from SARS-CoV-2, N protein from SARS-CoV-2, S protein from SARS-CoV, or S protein from MERS-CoV; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the S protein from SARS-CoV-2, S-RBD from SARS-CoV-2, S-NTD from SARS-CoV-2. N protein from SARS-CoV-2, S protein from SARS-CoV, and S protein from MERS-CoV. In embodiments, the one or more detection reagents specifically binds to the antibody biomarker. In embodiments, the detection reagent specifically binds IgA, IgG, or IgM. In embodiments, the one or more detection reagents comprises the S protein from SARS-CoV-2, S-RBD from SARS-CoV-2, S-NTD from SARS-CoV-2, N protein from SARS-CoV-2, S protein from SARS-CoV, and S protein from MERS-CoV. In embodiments, the one or more detection reagents comprises ACE2. In embodiments, the kit further comprises a surface.

In embodiments, the invention provides a kit comprising: (a) one or more binding reagents that specifically bind one or more antibody biomarkers, wherein each antibody biomarker specifically binds to: one or more of the HA protein from FluB/Brisbane/60/2008, HA protein from FluB/Phuket/3073/2013, HA protein from FluA/Michigan/45/2015 (H1N1), HA protein from FluA/HongKong/4801/2014 (H3N2), HA protein from FluA/Shanghai/2/2013 (H7N9), S protein from HCoV-HKU1, or S protein from HCoV-OC43; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the HA protein from FluB/Brisbane/60/2008, HA protein from FluB/Phuket/3073/2013, HA protein from FluA/Michigan/45/2015 (H1N1), HA protein from FluA/HongKong/4801/2014 (H3N2), HA protein from FluA/Shanghai/2/2013 (H7N9), S protein from HCoV-HKU1, and S protein from HCoV-OC43. In embodiments, the one or more detection reagents specifically binds to the antibody biomarker. In embodiments, the detection reagent specifically binds IgA, IgG, or IgM. In embodiments, the one or more detection reagents comprises the HA protein from FluB/Brisbane/60/2008, HA protein from FluB/Phuket/3073/2013, HA protein from FluA/Michigan/45/2015 (H1N1), HA protein from FluA/HongKong/4801/2014 (H3N2), HA protein from FluA/Shanghai/2/2013 (H7N9), S protein from HCoV-HKU1, and S protein from HCoV-OC43. In embodiments, the one or more detection reagents comprises ACE2. In embodiments, the kit further comprises a surface.

In embodiments, the invention provides a kit comprising: (a) one or more binding reagents that specifically bind one or more antibody biomarkers, wherein each antibody biomarker specifically binds to: one or more of the S protein from SARS-CoV-2, S-RBD from SARS-CoV-2, S-NTD from SARS-CoV-2, N protein from SARS-CoV-2, S protein from SARS-CoV, S protein from MERS-CoV, S protein from HCoV-HKU1, S protein from HCoV-OC43, or HA protein from FluA/HongKong/4801/2014 (H3N2); and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the S protein from SARS-CoV-2, S-RBD from SARS-CoV-2, S-NTD from SARS-CoV-2, N protein from SARS-CoV-2, S protein from SARS-CoV, S protein from MERS-CoV. S protein from HCoV-HKU1, S protein from HCoV-OC43, and HA protein from FluA/HongKong/4801/2014 (H3N2). In embodiments, the one or more detection reagents specifically binds to the antibody biomarker. In embodiments, the detection reagent specifically binds IgA, IgG, or IgM. In embodiments, the one or more detection reagents comprises the S protein from SARS-CoV-2, S-RBD from SARS-CoV-2, S-NTD from SARS-CoV-2, N protein from SARS-CoV-2, S protein from SARS-CoV, S protein from MERS-CoV, S protein from HCoV-HKU1, S protein from HCoV-OC43, and HA protein from FluA/HongKong/4801/2014 (H3N2). In embodiments, the one or more detection reagents comprises ACE2. In embodiments, the kit further comprises a surface.

In embodiments, the invention provides a kit comprising: (a) one or more binding reagents that specifically bind one or more antibody biomarkers, wherein each antibody biomarker specifically binds to one or more of the HA protein from influenza A H3, HA protein from influenza A H1, HA protein from influenza A H7, HA protein from influenza B/Phuket; or HA protein from influenza B/Brisbane; and (b) one or more detection reagent that specifically binds the one or more antibody biomarkers. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the HA protein from influenza B, HA protein from influenza A H1, HA protein from influenza A H3, and HA protein from influenza A H7. In embodiments, the kit further comprises a surface. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized influenza B/Brisbane HA protein. Spot 2 of FIG. 39B comprises an immobilized influenza A H7 (e.g., H7 Shanghai strain) HA protein, Spot 4 of FIG. 39B comprises an immobilized influenza A H1 (e.g., H1 Michigan strain) HA protein, Spot 8 of FIG. 39B comprises an immobilized influenza A H3 (e.g., H3/Hong Kong strain) protein, Spot 10 of FIG. 39B comprises an immobilized influenza B/Phuket HA protein, and Spots 3, 5, 6, 7, and 9 each comprises an immobilized BSA.

In embodiments, the invention provides a kit comprising: (a) one or more binding reagents that specifically bind one or more antibody biomarkers, wherein each antibody biomarker specifically binds to one or more of the HA protein from influenza A H3, HA protein from influenza A H1, HA protein from influenza A H7, HA protein from influenza B/Phuket, HA protein from influenza B/Brisbane, or F protein from RSV; and (b) one or more detection reagent that specifically binds the one or more antibody biomarkers. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the HA protein from influenza B/Phuket, the HA protein from influenza B/Brisbane, HA protein from influenza A/Michigan H1, HA protein from influenza A/Hong Kong H3, HA protein from influenza/Shanghai H7, and F protein from RSV. In embodiments, the one or more detection reagents specifically binds to the antibody biomarker. In embodiments, the detection reagent specifically binds IgA, IgG, or IgM. In embodiments, the one or more detection reagents comprises the HA protein from influenza B/Phuket, the HA protein from influenza B/Brisbane, HA protein from influenza A/Michigan H1, HA protein from influenza A/Hong Kong H3, HA protein from influenza/Shanghai H7, and F protein from RSV. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized influenza B/Brisbane HA protein, Spot 2 of FIG. 39B comprises an immobilized influenza A/Shanghai H7 HA protein, Spot 4 of FIG. 39B comprises an immobilized influenza A/Michigan H1 HA protein, Spot 7 of FIG. 39B comprises an immobilized RSV F protein, Spot 8 of FIG. 39B comprises an immobilized influenza A/Hong Kong H3 protein, Spot 10 of FIG. 39B comprises an immobilized influenza B/Phuket HA protein, and Spots 3, 5, 6, and 9 each comprises an immobilized BSA.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2. S protein from SARS-CoV-2, S protein from SARS-CoV, S protein from MERS-CoV, S protein from HCoV-OC43, S protein from HCoV-HKU1, S protein from NL63, S protein from HCoV-229E, or HA from influenza A H3; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2, S-RBD from SARS-CoV-2, S protein from SARS-CoV-2. S protein from SARS-CoV, S protein from MERS-CoV, S protein from HCoV-OC43, S protein from HCoV-HKU1, S protein from NL63, S protein from HCoV-229E, and HA from influenza A/Hong Kong H3. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2, S-RBD from SARS-CoV-2, S protein from SARS-CoV-2, S protein from SARS-CoV. S protein from MERS-CoV, S protein from HCoV-OC43. S protein from HCoV-HKU1, S protein from NL63, S protein from HCoV-229E, and HA from influenza A H3. In embodiments, the one or more detection reagents comprises ACE2. In embodiments, the kit further comprises a surface. In embodiments, the surface comprises two assay plates, wherein each assay plate is a 96-well plate.

In embodiments, the invention provides a kit comprising: (a) one or more binding reagents that specifically bind one or more antibody biomarkers, wherein each antibody biomarker specifically binds to: one or more of the N protein from SARS-CoV-2, S-NTD from SARS-CoV-2, S-RBD from SARS-CoV-2, S protein from SARS-CoV-2, S protein from SARS-CoV, S protein from MERS-CoV, S protein from HCoV-OC43, S protein from HCoV-HKU1, S protein from NL63, S protein from HCoV-229E, HA from influenza A H3, HA protein from influenza A H1, HA protein from influenza A H7, HA protein from influenza B/Phuket; HA protein from influenza B/Brisbane; or F protein from RSV; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, S-NTD from SARS-CoV-2. S-RBD from SARS-CoV-2, S protein from SARS-CoV-2, S protein from SARS-CoV, S protein from MERS-CoV, S protein from HCoV-OC43, S protein from HCoV-HKU1, S protein from NL63, S protein from HCoV-229E, HA from influenza A/Hong Kong H3, HA protein from influenza A/Michigan H1, HA protein from influenza A/Shanghai H7, HA protein from influenza B/Phuket; HA protein from influenza B/Brisbane; and F protein from RSV. In embodiments, the one or more detection reagents specifically binds to the antibody biomarker. In embodiments, the detection reagent specifically binds IgA, IgG, or IgM. In embodiments, the one or more detection reagents comprises the N protein from SARS-CoV-2, S-NTD from SARS-CoV-2, S-RBD from SARS-CoV-2, S protein from SARS-CoV-2, S protein from SARS-CoV, S protein from MERS-CoV, S protein from HCoV-OC43, S protein from HCoV-HKU1, S protein from NL63, S protein from HCoV-229E, HA from influenza A H3, HA protein from influenza A H1, HA protein from influenza A H7, HA protein from influenza B/Phuket; HA protein from influenza B/Brisbane; and F protein from RSV. In embodiments, the one or more detection reagents comprises ACE2. In embodiments, the kit further comprises a surface. In embodiments, the surface comprises two assay plates, wherein each assay plate is a 96-well plate.

In embodiments, the invention provides a kit comprising: (a) one or more binding reagents that specifically bind one or more antibody biomarkers, wherein each antibody biomarker specifically binds to: one or more of the N protein from SARS-CoV-2 or the S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2 and S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises the N protein from SARS-CoV-2 and S-RBD from SARS-CoV-2. In embodiments, the one or more detection reagents comprises ACE2. In embodiments, the kit further comprises a surface. In embodiments, the kit comprises one or more, two or more, three or more, or four or more surfaces. In embodiments, the kit comprises one or more surfaces, wherein each surface comprises 1, 2, 3, 4, 5, 6, 7, 8, 9, or 10 binding domains.

In embodiments, the invention provides a kit comprising: (a) one or more binding reagents that specifically bind a SARS-CoV-2 protein and a host inflammatory and/or tissue damage response biomarker; and (b) one or more detection reagents. In embodiments, the SARS-CoV-2 protein is SARS-CoV-2 S protein, SARS-CoV-2 N protein, or both. In embodiments, the host biomarker is GM-CSF, Granzyme A. Granzyme B, IFN-α2a, IFN-β, IFN-γ, IL-1β, IL-1RA, IL-4, IL-5, IL-6, IL-7, IL-8, IL-9, IL-12p70, IP-10, I-TAC, MCP-1, MCP-2, MCP-4, MDC, MIP-1α, MIP-1β, TNF-α, VEGF-A, or a combination thereof. In embodiments, the IgG, IgA, and/or IgM is from a human, mouse, rat, ferret, minx, bat, or combination thereof. In embodiments, the kit further comprises a surface. In embodiments, the kit comprises one or more, two or more, three or more, or four or more surfaces. In embodiments, the kit comprises one or more surfaces, wherein each surface comprises 1, 2, 3, 4, 5, 6, 7, 8, 9, or 10 binding domains.

In embodiments, the invention provides a kit comprising: (a) one or more binding reagents that specifically bind one or more antibody biomarkers and a host inflammatory and/or tissue damage response biomarker; and (b) one or more detection reagents. In embodiments, the antibody biomarker binds SARS-CoV-2 S protein, SARS-CoV-2 N protein, or both. In embodiments, the host biomarker is GM-CSF, Granzyme A, Granzyme B, IFN-α2a, IFN-β, IFN-γ, IL-1β, IL-1RA, IL-4, IL-5, IL-6, IL-7, IL-8, IL-9, IL-12p70, IP-10, I-TAC, MCP-1, MCP-2, MCP-4, MDC. MIP-1α, MIP-1β, TNF-α, VEGF-A, or a combination thereof. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the N protein from SARS-CoV-2, the S protein from SARS-CoV-2, or both. In embodiments, the one or more detection reagents specifically binds to the antibody biomarker. In embodiments, the one or more detection reagents specifically binds IgA, IgG, or IgM. In embodiments, the one or more detection reagents comprises the N protein from SARS-CoV-2, the S protein from SARS-CoV-2, or both. In embodiments, the one or more detection reagents comprises ACE2. In embodiments, the kit further comprises a surface. In embodiments, the kit comprises one or more, two or more, three or more, or four or more surfaces. In embodiments, the kit comprises one or more surfaces, wherein each surface comprises 1, 2, 3, 4, 5, 6, 7, 8, 9, or 10 binding domains.

In embodiments, the surface of the kits described herein comprises a multi-well assay plate. In embodiments, the surface comprises avidin or streptavidin. In embodiments, each binding reagent comprises biotin. In embodiments, the surface comprises a targeting agent. In embodiments, the kit further comprises a linking agent connected to a targeting agent complement. In embodiments, each binding reagent comprises a supplemental linking agent. In embodiments, the targeting agent and targeting agent complement comprise complementary oligonucleotides. In embodiments, the linking agent comprises avidin or streptavidin, and the supplemental linking agent comprises biotin. Targeting agents, targeting agent complements, linking agents, and supplemental linking agents are further described herein.

In embodiments, the invention provides a kit comprising: (a) one or more binding reagents, each binding reagent binding specifically to: (1) a viral component; (2) a host antibody biomarker; or (3) a host inflammatory and/or tissue damage response biomarker; and (b) one or more detection reagents, each detection reagent binding specifically to the viral component, host antibody biomarker, or the host inflammatory and/or tissue damage response biomarker. In embodiments, the detection reagent that binds to the host antibody biomarker binds IgA. IgG, or IgM. In embodiments, the detection reagent is ACE2. In embodiments, the viral component is a viral protein. In embodiments, the viral component is a viral nucleic acid. In embodiments, the virus is a coronavirus. In embodiments, the virus is SARS-CoV-2. In embodiments, the kit further comprises a surface.

In embodiments, the invention provides a combination of any of the kits described herein. In embodiments, the combination of kits is provided as a single kit, comprising the components of each of the individual kits.

In embodiments, the binding reagent is an antibody or antigen-binding fragment thereof. In embodiments, the detection reagent is an antibody or antigen-binding fragment thereof. In embodiments, any of the detection reagents described herein comprises a detectable label as described herein. In embodiments, the detection reagent comprises a nucleic acid probe as described herein. In embodiments, the kit comprises first and second detection reagents, and the first and second detection reagents respectively comprise first and second nucleic acid probes as described herein. In embodiments, the kit further comprises a reagent for conjugating the detection reagent to a detectable label or a nucleic acid probe.

In embodiments, the detection reagent is lyophilized. In embodiments, the detection reagent is provided in solution. In embodiments, the binding reagent is immobilized on the binding domain. In embodiments, the binding reagent is provided in solution. In embodiments, the reagents and other components of the kit are provided separately. In embodiments, they are provided separately according to their optimal shipping or storage temperatures.

Reagents and methods for immobilizing binding reagents to surfaces, e.g., via targeting agents/targeting agent complements, linking agents/supplemental linking agents, and bridging agents are described herein. In embodiments, the surface is a plate. In embodiments, the surface is a multi-well plate. Non-limiting examples of plates include the MSD9® SECTOR™ and MSD QUICKPLEX® assay plates. e.g., MSD® GOLD™ 96-well Small Spot Streptavidin plate. In embodiments, the surface is a particle. In embodiments, the particle comprises a paramagnetic bead. In embodiments, the surface is a cartridge. In embodiments, the surface comprises an electrode. In embodiments, the electrode is a carbon ink electrode.

In embodiments, the kit further comprises a calibration reagent. In embodiments, the calibration reagent comprises a known quantity of the virus, viral component, or biomarker as described herein. In embodiments, multiple calibration reagents comprise a range of concentrations of the virus, viral component, or biomarker. In embodiments, the multiple calibration reagents comprise concentrations of the virus, viral component, or biomarker near the upper and lower limits of quantitation for the immunoassay. In embodiments, the multiple concentrations of the calibration reagent span the entire dynamic range of the immunoassay. In embodiments, the calibration reagent comprises an antibody biomarker. In embodiments, the antibody biomarker is a neutralizing antibody as described herein. In embodiments, the neutralizing antibody is a monoclonal antibody. In embodiments, the calibration reagent comprises a neutralizing antibody that specifically binds the SARS-CoV S protein, the SARS-CoV-2 S protein, or both. In embodiments, the calibration reagent is derived from human serum known to contain one or more antibodies that specifically bind to one or more viral antigens described herein. In embodiments, the one or more antibodies is human IgG, human IgM, or a combination thereof. In embodiments, the calibration reagent comprises an antibody that specifically binds the SARS-CoV S protein, an antibody that specifically binds SARS-CoV-2 S-NTD, an antibody that specifically binds SARS-CoV-2 S protein, an antibody that specifically binds SARS-CoV-2 S-RBD, an antibody that specifically binds SARS-CoV-2 N protein, an antibody that specifically binds HCoV-OC43 S protein, an antibody that specifically binds HCoV-HKU1 S protein, an antibody that specifically binds MERS-CoV S protein, an antibody that specifically binds HCoV-NL63 S protein, an antibody that specifically binds HCoV-229E S protein, an antibody that specifically binds influenza A/Hong Kong H3 HA protein, an antibody that specifically binds influenza B/Brisbane HA protein, an antibody that specifically binds influenza A/Shanghai H7 HA protein, an antibody that specifically binds influenza A/Michigan H1 HA protein, an antibody that specifically binds influenza B/Phuket HA protein, and an antibody that specifically binds RSV pre-fusion F protein. In embodiments, the calibration reagent comprises an IgG that specifically binds to SARS-CoV-2 S protein, an IgG that specifically binds to SARS-CoV-2 N protein, an IgG that specifically binds to SARS-CoV-2 S-RBD, an IgM that specifically binds to SARS-CoV-2 S protein, an IgM that specifically binds to SARS-CoV-2 N protein, an IgM that specifically binds to SARS-CoV-2 S-RBD, an IgA that specifically binds to SARS-CoV-2 S protein, an IgA that specifically binds to SARS-CoV-2 N protein, and an IgA that specifically binds to SARS-CoV-2 S-RBD.

In embodiments, the calibration reagents are provided in the kit at the following concentrations: about 1 to about 10 BAU/mL of an IgG that specifically binds to SARS-CoV-2 S protein, about 0.1 to about 5 BAU/mL of an IgG that specifically binds to SARS-CoV-2 N protein, about 5 to about 20 BAU/mL of an IgG that specifically binds to SARS-CoV-2 S-RBD, about 0.1 to about 2 BAU/mL of an IgM that specifically binds to SARS-CoV-2 S protein, about 1 to about 5 BAU/mL of an IgM that specifically binds to SARS-CoV-2 N protein, about 0.1 to about 2 BAU/mL of an IgM that specifically binds to SARS-CoV-2 S-RBD, about 1 to about 5 BAU/mL of an IgA that specifically binds to SARS-CoV-2 S protein, about 1 to about 10 BAU/mL of an IgA that specifically binds to SARS-CoV-2 N protein, and about 0.1 to about 5 BAU/mL of an IgA that specifically binds to SARS-CoV-2 S-RBD. Concentrations of the calibration reagents provided herein are defined according to the “First WHO International Standard for anti-SARS-CoV-2 immunoglobulin” (NIBSC code: 20/136).

In embodiments, the calibration reagents are provided in the kit at the following concentrations: about 6.31 BAU/mL of an IgG that specifically binds to SARS-CoV-2 S protein, about 1.89 BAU/mL of an IgG that specifically binds to SARS-CoV-2 N protein, about 8.16 BAU/mL of an IgG that specifically binds to SARS-CoV-2 S-RBD, about 0.867 BAU/mL of an IgM that specifically binds to SARS-CoV-2 S protein, about 2.64 BAU/mL of an IgM that specifically binds to SARS-CoV-2 N protein, about 0.466 BAU/mL of an IgM that specifically binds to SARS-CoV-2 S-RBD, about 3.09 BAU/mL of an IgA that specifically binds to SARS-CoV-2 S protein, about 5.57 BAU/mL of an IgA that specifically binds to SARS-CoV-2 N protein, and about 1.56 BAU/mL of an IgA that specifically binds to SARS-CoV-2 S-RBD.

In embodiments, the calibration reagent is a positive control reagent. In embodiments, the calibration reagent is a negative control reagent. In embodiments, the positive or negative control reagent is used to provide a basis of comparison for the biological sample to be tested with the methods of the present invention. In embodiments, the positive control reagent comprises multiple concentrations of the virus, viral component, or biomarker. In embodiments, the positive control reagent comprises an antibody. In embodiments, the positive control reagent comprises human IgG, IgM, IgA, or a combination thereof. In embodiments, the positive control reagent comprises an antibody that specifically binds the SARS-CoV-2 S protein, SARS-CoV-2 N protein, SARS-CoV-2 S-RBD, or a combination thereof. In embodiments, the positive control reagent comprises an IgG that specifically binds to SARS-CoV-2 S protein, an IgG that specifically binds to SARS-CoV-2 N protein, an IgG that specifically binds to SARS-CoV-2 S-RBD, an IgM that specifically binds to SARS-CoV-2 S protein, an IgM that specifically binds to SARS-CoV-2 N protein, an IgM that specifically binds to SARS-CoV-2 S-RBD, an IgA that specifically binds to SARS-CoV-2 S protein, an IgA that specifically binds to SARS-CoV-2 N protein, and an IgA that specifically binds to SARS-CoV-2 S-RBD.

In embodiments, the positive control reagent is provided in the kit at the following concentrations: about 0.005 to about 1 BAU/mL of an IgG that specifically binds to SARS-CoV-2 S protein; about 0.001 to about 0.1 BAU/mL of an IgG that specifically binds to SARS-CoV-2 N protein; about 0.005 to about 1 BAU/mL of an IgG that specifically binds to SARS-CoV-2 S-RBD; about 0.001 to about 0.1 BAU/mL of an IgM that specifically binds to SARS-CoV-2 S protein; about 0.01 to about 0.1 BAU/mL of an IgM that specifically binds to SARS-CoV-2 N protein; about 0.001 to about 0.1 BAU/mL of an IgM that specifically binds to SARS-CoV-2 S-RBD; about 0.005 to about 0.5 BAU/mL of an IgA that specifically binds to SARS-CoV-2 S protein; about 0.005 to about 0.5 BAU/mL of an IgA that specifically binds to SARS-CoV-2 N protein; and about 0.001 to about 0.1 BAU/mL of an IgA that specifically binds to SARS-CoV-2 S-RBD. In embodiments, the positive control reagents are provided in the kit at the following concentrations: about 0.1504, about 0.0372, and about 0.0133 BAU/mL of an IgG that specifically binds to SARS-CoV-2 S protein; about 0.0457, about 0.0078, and about 0.0025 BAU/mL of an IgG that specifically binds to SARS-CoV-2 N protein; about 0.1952, about 0.0576, and about 0.0148 BAU/mL of an IgG that specifically binds to SARS-CoV-2 S-RBD; about 0.0187, about 0.0054, and about 0.0077 BAU/mL of an IgM that specifically binds to SARS-CoV-2 S protein; about 0.061, about 0.030, and about 0.0285 BAU/mL of an IgM that specifically binds to SARS-CoV-2 N protein; about 0.011, about 0.0047, and about 0.0068 BAU/mL of an IgM that specifically binds to SARS-CoV-2 S-RBD; about 0.0768, about 0.023, and about 0.0103 BAU/mL of an IgA that specifically binds to SARS-CoV-2 S protein; about 0.1414, about 0.0237, and about 0.0379 BAU/mL of an IgA that specifically binds to SARS-CoV-2 N protein; and about 0.0394, about 0.0131, and about 0.0085 BAU/mL of an IgA that specifically binds to SARS-CoV-2 S-RBD.

In embodiments, the calibration reagent is lyophilized. In embodiments, the calibration reagent is provided in solution. In embodiments, the calibration reagent is provided as a stock concentration that is 5×, 10×, 20×, 30×, 40×, 50×, 60×, 70×, 80×, 90×, 100×, 125×, 150× or higher fold concentrations of the highest working concentration of the calibration reagent. In embodiments, the kit further comprises a diluent for preparing multiple concentrations of the calibration reagent. In embodiments, the calibration reagent provided in the kit is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. In embodiments, the kit comprises multiple calibration reagents at multiple concentrations, e.g., two or more, three or more, four or more, or five or more concentrations. In embodiments, the multiple concentrations of calibration reagents are used to calculate a standard curve. In embodiments, the multiple concentrations of calibration reagents provide thresholds indicating low, medium, or high levels of the virus, viral component, or biomarker being measured.

In embodiments, the kit further comprises a sample collection device. In embodiments, the sample collection device is an applicator stick. In embodiments, the sample collection device is a swab. In embodiments, the sample collection device is a tissue scraper. In embodiments, the sample collection device is a vial or container for collecting a liquid sample.

In embodiments, the kit further comprises one or more of a buffer, e.g., assay buffer, reconstitution buffer, storage buffer, read buffer, wash buffer and the like; a diluent; a blocking solution; an assay consumable, e.g., assay modules, vials, tubes, liquid handling and transfer devices such as pipette tips, covers and seals, racks, labels, and the like; an assay instrument; and/or instructions for carrying out the assay.

In embodiments, the kit comprises lyophilized reagents, e.g., detection reagent and/or calibration reagent. In embodiments, the kit comprises one or more solutions to reconstitute the lyophilized reagents.

In embodiments, a kit comprising the components above include stock concentrations of the components that are 5×, 10×, 20×, 30×, 40×, 50×, 60×, 70×, 80×, 90×, 100×, 125×, 150× or higher fold concentrations of the working concentrations of the immunoassays herein.

In embodiments, the invention provides a kit for collecting a biological sample. In embodiments, the kit for collecting a biological sample can be provided to a subject for collecting the subject's own sample, e.g., saliva sample. The collected sample can then be provided by the subject, e.g., delivered in person or via postal service, to a laboratory for analysis. In embodiments, the kit further comprises an assay instrument. e.g., an assay cartridge and/or a cartridge reader, for the subject to analyze the collected sample. In embodiments, the kit comprises a sample collection device, e.g., an applicator stick, a swab, a tissue scraper, or a vial or container for collecting a liquid sample. In embodiments, the sample collection device comprises a straw for collecting a saliva sample. In embodiments, the sample collection device comprises a storage solution that stabilizes the sample. In embodiments, the sample collection device comprises a unique sample identifier, e.g., a barcode. In embodiments, the kit further comprises instructions for collecting the sample and/or for analyzing the sample in an assay instrument. In embodiments, the kit further comprises an absorbent material, e.g., a tissue. In embodiments, the kit further comprises a secondary container (e.g., a bag) to secure the sample collection device. In embodiments, the kit further comprises a pre-paid postage label or a pre-paid envelope or box for mailing the collected sample.

All references cited herein, including patents, patent applications, papers, textbooks and the like, and the references cited therein, to the extent that they are not already, are hereby incorporated herein by reference in their entirety.

### EXEMPLARY EMBODIMENTS

The following Group A, Group B. and Group C are exemplary embodiments of the inventions disclosed herein.

### Group A Exemplary Embodiments

A kit for detecting multiple antibody biomarkers of interest in a sample, the kit comprising, in one or more vials, containers, or compartments: (a) one or more surfaces comprising multiple binding domains, wherein each of: an N protein from SARS-CoV-2, an S protein SARS-CoV-2, an S-RBD from SARS-CoV-2, an S-NTD from SARS-CoV-2, an S protein from SARS-CoV, an S protein from MERS-CoV, an S protein from HCoV-HKU1, an S protein from HCoV-OC43, an HA protein from FluB/Brisbane, an HA protein from FluB/Phuket, an HA protein from FluA H1/Michigan, an HA protein from FluA H3/Hong Kong, and an HA protein from FluA H7/Shanghai strain is immobilized on a distinct binding domain on the one or more surfaces; and (b) one or more detection reagents, wherein the detection reagent is an antibody, a viral antigen, or a detectable competitor.

A method for detecting multiple antibody biomarkers of interest in a sample, the method comprising: (a) contacting the biological sample with a surface comprising multiple binding domains, wherein each of an N protein from SARS-CoV-2, an S protein SARS-CoV-2, an S-RBD from SARS-CoV-2, an S-NTD from SARS-CoV-2, an S protein from SARS-CoV, an S protein from MERS-CoV, an S protein from HCoV-HKU1, an S protein from HCoV-OC43, an HA protein from FluB/Brisbane, an HA protein from FluB/Phuket, an HA protein from FluA H1/Michigan, an HA protein from FluA H3/Hong Kong, and an HA protein from FluA H7/Shanghai strain is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with a detection reagent, wherein the detection reagent is an antibody, a viral antigen, or a detectable competitor; and (d) measuring concentration of the antibody biomarker in each binding complex.

A kit for detecting a viral infection immune response in a sample, the kit comprising, in one or more vials, containers, or compartments: (a) at least a first binding reagent for a viral antigen and at least a second binding reagent for a biomarker, wherein the first and second binding reagents are immobilized or capable of being immobilized on distinct binding domains on a surface, wherein the viral antigen is a SARS-CoV-2 N protein, a SARS-CoV-2 S protein, or a combination thereof, and wherein the biomarker is GM-CSF. Granzyme A, Granzyme B, IFN-α2a, IFN-β, IFN-γ, IL-1β, IL-1RA, IL-4, IL-5, IL-6, IL-7, IL-8, IL-9, IL-12p70, IP-10, I-TAC, MCP-1, MCP-2, MCP-4, MDC, MIP-1α, MIP-1β, TNF-α, VEGF-A, or a combination thereof, and (b) at least a first detection reagent for the viral antigen and at least a second detection reagent for the biomarker.

A multiplexed method for determining a viral infection immune response in a sample, the method comprising quantifying the amounts of at least one viral antigen and at least one biomarker in the biological sample, wherein the at least one viral antigen is a SARS-CoV-2 N protein, a SARS-CoV-2 S protein, or a combination thereof, and wherein the at least one biomarker is GM-CSF, Granzyme A, Granzyme B, IFN-α2a, IFN-β, IFN-γ, IL-1β, IL-1RA, IL-4, IL-5, IL-6, IL-7, IL-8, IL-9, IL-12p70, IP-10, I-TAC, MCP-1, MCP-2, MCP-4, MDC, MIP-1α, MIP-1β, TNF-α, VEGF-A, or a combination thereof, wherein the quantifying comprises: (a) contacting the biological sample with a surface comprising at least a first binding domain and a second binding domain, wherein the first binding domain comprises a binding reagent for the at least one viral antigen, and the second binding domain comprises a binding reagent for the at least one biomarker, thereby forming a binding complex in each binding domain; and (b) detecting the binding complex in each binding domain.

A kit for detecting an EV from a SARS-CoV-2 infected cell in a sample, the kit comprising, in one or more vials, containers, or compartments: (a) a first binding reagent that is immobilized or capable of being immobilized on a surface; and (b) a second binding reagent that comprises a nucleic acid probe; wherein one of the first or second binding reagents binds to an EV surface marker, and the other of the first or second binding reagents binds to a SARS-CoV-2 viral antigen.

A method for detecting an EV from a SARS-CoV-2 infected cell in a sample, the method comprising: (a) contacting the sample with (i) a first binding reagent that is immobilized or capable of being immobilized on a surface, and (ii) a second binding reagent that comprises a nucleic acid probe, wherein one of the first or second binding reagents binds to an EV surface marker, and the other of the first or second binding reagents binds to a SARS-CoV-2 viral antigen, thereby forming a binding complex on the surface comprising the EV and the first and second binding reagents; (b) extending the nucleic acid probe to form an extended oligonucleotide; and (c) measuring the amount of the extended oligonucleotide, thereby detecting the EV from the SARS-CoV-2 infected cell.

A kit for detecting an intact SARS-CoV-2 virus a sample, the kit comprising, in one or more vials, containers, or compartments: (a) a binding reagent that specifically binds a first viral antigen on a viral surface, wherein the binding reagent is immobilized or capable of being immobilized on distinct binding domains on a surface; (b) a first detection reagent that binds to a second viral antigen on the viral surface, wherein the first detection reagent comprises a first nucleic acid probe; and (c) a second detection reagent that binds to a third viral antigen on the viral surface, wherein the second detection reagent comprises a second nucleic acid probe.

A method for detecting an intact SARS-CoV-2 virus in a sample, the method comprising: (a) contacting the biological sample with (i) a binding reagent that specifically binds a first viral antigen on a viral surface, wherein the binding reagent is immobilized or capable of being immobilized on distinct binding domains on a surface; (ii) a first detection reagent that binds to a second viral antigen on the viral surface, wherein the first detection reagent comprises a first nucleic acid probe; and (iii) a second detection reagent that binds to a third viral antigen on the viral surface, wherein the second detection reagent comprises a second nucleic acid probe, thereby forming a binding complex on the surface comprising the intact SARS-CoV-2 virus, the binding reagent, and the first and second detection reagents; (b) using an extension process that requires the first and second nucleic acid probes to be in proximity, extending the second nucleic acid probe to form an extended oligonucleotide; and (c) measuring the amount of extended oligonucleotide, thereby detecting the intact SARS-CoV-2 virus in the biological sample.

A kit for detecting a SARS-CoV-2 nucleic acid in a sample, the kit comprising, in one or more vials, containers, or compartments: (a) an oligonucleotide binding reagent that comprises (i) a targeting agent complement (TAC); (ii) an amplification primer; (iii) a hybridization region comprising a complementary sequence to the SARS-CoV-2 nucleic acid; and (iv) an amplification blocker; and (b) a site-specific nuclease that forms a complex with the oligonucleotide binding reagent.

A method for detecting a SARS-CoV-2 nucleic acid in a sample, comprising: (a) contacting the sample with an oligonucleotide binding reagent, wherein the oligonucleotide binding reagent comprises: (i) a targeting agent complement (TAC); (ii) an amplification primer; (iii) a hybridization region comprising a complementary sequence to the SARS-CoV-2 nucleic acid; and (iv) an amplification blocker; (b) forming a binding complex comprising the SARS-CoV-2 nucleic acid and the oligonucleotide binding reagent; (c) contacting the binding complex with a site-specific nuclease that cleaves the oligonucleotide binding reagent to remove the amplification blocker therefrom, thereby generating a first cleaved oligonucleotide comprising the targeting agent complement and the amplification primer, wherein the first cleaved oligonucleotide is not bound to the SARS-CoV-2 nucleic acid; (d) immobilizing the first cleaved oligonucleotide to a detection surface comprising a targeting agent, wherein the targeting agent is a binding partner of the targeting agent complement; (e) extending the first cleaved oligonucleotide on the detection surface to form an extended oligonucleotide; and (f) detecting the extended oligonucleotide, thereby detecting the SARS-CoV-2 nucleic acid in the sample.

A kit for detecting a SARS-CoV-2 nucleic acid in a sample, the kit comprising, in one or more vials, containers, or compartments: (a) an oligonucleotide detection reagent that comprises (i) a targeting agent complement (TAC); (ii) an amplification primer; and (iii) an amplification blocker; and (b) a site-specific nuclease having collateral activity.

A method for detecting a SARS-CoV-2 nucleic acid in a sample, comprising: (a) contacting the sample with a site-specific nuclease comprising collateral cleavage activity and an oligonucleotide detection reagent, wherein the oligonucleotide detection reagent comprises: (i) a targeting agent complement (TAC); (ii) an amplification primer; and (iii) an amplification blocker; wherein the site-specific nuclease binds to the SARS-CoV-2 nucleic acid and collaterally cleaves the oligonucleotide detection reagent to remove the amplification blocker therefrom, thereby generating a first cleaved oligonucleotide comprising the targeting agent complement and the amplification primer; (b) immobilizing the first cleaved oligonucleotide to a detection surface comprising a targeting agent, wherein the targeting agent is a binding partner of the targeting agent complement; (c) extending the first cleaved oligonucleotide to form an extended oligonucleotide; and (d) detecting the extended oligonucleotide, thereby detecting the SARS-CoV-2 nucleic acid in the sample.

A kit for detecting a SARS-CoV-2 nucleic acid in a sample, the kit comprising, in one or more vials, containers, or compartments: (a) a targeting probe that is complementary to a first region of the SARS-CoV-2 nucleic acid in the sample, wherein the targeting probe comprises an oligonucleotide tag, and wherein the oligonucleotide tag is complementary to a binding reagent immobilized on a surface; (b) a detection probe that is complementary to a second region that is adjacent to the first region of the SARS-CoV-2 nucleic acid, wherein the detection probe comprises a detectable label.

A method for detecting a SARS-CoV-2 nucleic acid in a sample, comprising: (a) contacting the sample with: (i) a targeting probe that is complementary to a first region of the SARS-CoV-2 nucleic acid in the sample, wherein the targeting probe comprises an oligonucleotide tag; and (ii) a detection probe that is complementary to a second region that is adjacent to the first region of the SARS-CoV-2 nucleic acid, wherein the detection probe comprises a detectable label; (b) hybridizing the targeting and detection probes to the SARS-CoV-2 nucleic acid; (c) ligating the targeting and detection probes that hybridize with perfect complementarity to the first and second regions of the SARS-CoV-2 nucleic acid to form a ligated target complement comprising the oligonucleotide tag and the detectable label; (d) contacting the product of (c) with a surface comprising a binding reagent immobilized in one or more binding domains, wherein the binding reagent comprises an oligonucleotide complementary to the oligonucleotide tag; (e) forming a binding complex on the surface comprising the binding reagent and the ligated target complement; and (0) detecting the binding complex, thereby detecting the SARS-CoV-2 nucleic acid.

The kit or method of any one of the above exemplary embodiments of Group A, wherein the sample comprises a respiratory sample.

The kit or method of any one of the above exemplary embodiments of Group A, wherein the sample comprises wastewater.

The kit or method of any one of the above exemplary embodiments of Group A, wherein the sample is obtained from two or more individuals.

### Group B Exemplary Embodiments

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-NTD from SARS-CoV-2, an S-RBD from SARS-CoV-2, an S protein from SARS-CoV-2, and an S protein from SARS-CoV;
(ii) an N protein from SARS-CoV-2, an S-RBD from SARS-CoV-2, and an S protein from SARS-CoV-2;
(iii) a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2;
(iv) a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2;
(v) a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
(vi) a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
or
(vii) a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2
is immobilized on a distinct binding domain on the surface; and (b) one or more detection reagents, wherein each detection reagent is an antibody or a viral antigen.

A method for detecting multiple antibody biomarkers of interest in a sample, the method comprising: (a) contacting the biological sample with a surface comprising multiple binding domains, wherein each of:

(i) an N protein from SARS-CoV-2, an S-NTD from SARS-CoV-2, an S-RBD from SARS-CoV-2, an S protein from SARS-CoV-2, and an S protein from SARS-CoV;
(ii) an N protein from SARS-CoV-2, an S-RBD from SARS-CoV-2, and an S protein from SARS-CoV-2;
(iii) a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2;
(iv) a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2;
(v) a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
(vi) a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
or
(vii) a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with a detection reagent, wherein the detection reagent is an antibody or a viral antigen; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-RBD from SARS-CoV-2, and an S protein from SARS-CoV-2;
or
(ii) a wild-type S protein from SARS-CoV-2, an N protein from SARS-CoV-2, an S-RBD from SARS-CoV-2 strain 501Y.V2, and an S protein from SARS-CoV-2 strain 501Y.V2 is immobilized on a distinct binding domain on the surface; and (b) one or more detection reagents, wherein each detection reagent is an antibody or a viral antigen.

A method for detecting multiple antibody biomarkers of interest in a sample, the method comprising: (a) contacting the biological sample with a surface comprising multiple binding domains, wherein each of.

(i) an N protein from SARS-CoV-2, an S-RBD from SARS-CoV-2, and an S protein from SARS-CoV-2;
or
(ii) a wild-type S protein from SARS-CoV-2, an N protein from SARS-CoV-2, an S-RBD from SARS-CoV-2 strain 501Y.V2, and an S protein from SARS-CoV-2 strain 501Y.V2 is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with a detection reagent, wherein the detection reagent is an antibody or a viral antigen; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and an HA from influenza A/Hong Kong H3;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and an S protein from HCoV-229E;
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and an S protein from HCoV-229E;
(iv) an N protein from SARS-CoV-2, an S-RBD from SARS-CoV-2, an S protein from SARS-CoV-2, an S2 from SARS-CoV-2, an S protein from SARS-CoV, an S2 from SARS-CoV-2, an S protein from HCoV-OC43, an S protein from HCoV-HKU1, an S protein from HCoV-NL63, and an S protein from HCoV-229E;
(v) an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2;
or
(vi) an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2
is immobilized on a distinct binding domain on the surface; and (b) one or more detection reagents, wherein each detection reagent is an antibody or a viral antigen.

A method for detecting multiple antibody biomarkers of interest in a sample, the method comprising: (a) contacting the biological sample with a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and an HA from influenza A/Hong Kong H3;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and an S protein from HCoV-229E;
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and an S protein from HCoV-229E;
(iv) an N protein from SARS-CoV-2, an S-RBD from SARS-CoV-2, an S protein from SARS-CoV-2, an S2 from SARS-CoV-2, an S protein from SARS-CoV, an S2 from SARS-CoV-2, an S protein from HCoV-OC43, an S protein from HCoV-HKU1, an S protein from HCoV-NL63, and an S protein from HCoV-229E;
(v) an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2;
or
(vi) an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with a detection reagent, wherein the detection reagent is an antibody or a viral antigen; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
or
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza/Shanghai H7, an HA from influenza B/Phuket, an HA from influenza B/Brisbane, and an F protein from RSV
is immobilized on a distinct binding domain on the surface; and (b) one or more detection reagents, wherein each detection reagent is an antibody or a viral antigen.

A method for detecting multiple antibody biomarkers of interest in a sample, the method comprising: (a) contacting the biological sample with a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV:
or
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza/Shanghai H7, an HA from influenza B/Phuket, an HA from influenza B/Brisbane, and an F protein from RSV
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with a detection reagent, wherein the detection reagent is an antibody or a viral antigen; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of an HA protein from influenza A/Hong Kong H3, an HA protein from influenza A/Michigan H1, an HA protein from influenza A/Shanghai H7, an HA protein from influenza B/Phuket; an HA protein from influenza B/Brisbane; and an F protein from RSV is immobilized on a distinct binding domain on the surface; and (b) one or more detection reagents, wherein each detection reagent is an antibody or a viral antigen.

A method for detecting multiple antibody biomarkers of interest in a sample, the method comprising: (a) contacting the biological sample with a surface comprising multiple binding domains, wherein each of: an HA protein from influenza A/Hong Kong H3, an HA protein from influenza A/Michigan H1, an HA protein from influenza A/Shanghai H7, an HA protein from influenza B/Phuket; an HA protein from influenza B/Brisbane; and an F protein from RSV is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with a detection reagent, wherein the detection reagent is an antibody or a viral antigen; and (d) measuring concentration of the antibody biomarker in each binding complex.

A kit for determining a viral infection response in a sample, the kit comprising, in one or more vials, containers, or compartments: (a) a first binding reagent and a first detection reagent that specifically bind GFAP; (b) a second binding reagent and a second detection reagent that specifically bind Tau; and (c) a third binding reagent and a third detection reagent that specifically bind NF-L.

A multiplexed method for determining a viral infection response in a sample, the method comprising quantifying the amounts of GFAP, Tau. and NF-L in the sample, wherein the quantifying comprises: (a) contacting the sample with (i) a surface comprising at least first, second, and third binding domains, wherein the first binding domain comprises a first binding reagent for GFAP, the second binding domain comprises a second binding reagent for Tau, and the third binding domain comprises a third binding reagent for NF-L, thereby forming a binding complex in each binding domain; and (b) detecting the binding complex in each binding domain.

### Group C Exemplary Embodiments

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-NTD from SARS-CoV-2, an S-RBD from SARS-CoV-2, an S protein from SARS-CoV-2, and an S protein from SARS-CoV;
(ii) an N protein from SARS-CoV-2, an S-RBD from SARS-CoV-2, and an S protein from SARS-CoV-2;
(iii) a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2;
(iv) a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2;
(v) a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2:
(vi) a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
or
(vii) a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2
is immobilized on a distinct binding domain on the surface; and (b) an ACE2 detection reagent.

A method for detecting multiple antibody biomarkers of interest in a sample, the method comprising: (a) contacting the biological sample with a surface comprising multiple binding domains, wherein each of:

(i) an N protein from SARS-CoV-2, an S-NTD from SARS-CoV-2, an S-RBD from SARS-CoV-2, an S protein from SARS-CoV-2, and an S protein from SARS-CoV;
(ii) an N protein from SARS-CoV-2, an S-RBD from SARS-CoV-2, and an S protein from SARS-CoV-2;
(iii) a wild-type S protein from SARS-CoV-2, an Orf8 oligomer from SARS-CoV-2, an N protein from SARS-CoV-2, a Mem protein from SARS-CoV-2, an Orf7a protein from SARS-CoV-2, an Env protein from SARS-CoV-2, an Orf8 monomer from SARS-CoV-2, and an S-RBD from SARS-CoV-2;
(iv) a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2;
(v) a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
(vi) a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2;
or
(vii) a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with an ACE2 detection reagent; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-RBD from SARS-CoV-2, and an S protein from SARS-CoV-2;
or
(ii) a wild-type S protein from SARS-CoV-2, an N protein from SARS-CoV-2, an S-RBD from SARS-CoV-2 strain 501Y.V2, and an S protein from SARS-CoV-2 strain 501Y.V2
is immobilized on a distinct binding domain on the surface; and (b) an ACE2 detection reagent. A method for detecting multiple antibody biomarkers of interest in a sample, the method comprising: (a) contacting the biological sample with a surface comprising multiple binding domains, wherein each of:
(i) an N protein from SARS-CoV-2, an S-RBD from SARS-CoV-2, and an S protein from SARS-CoV-2;
or
(ii) a wild-type S protein from SARS-CoV-2, an N protein from SARS-CoV-2, an S-RBD from SARS-CoV-2 strain 501Y.V2, and an S protein from SARS-CoV-2 strain 501Y.V2
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with an ACE2 detection reagent; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and an HA from influenza A/Hong Kong H3;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and an S protein from HCoV-229E;
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and an S protein from HCoV-229E;
(iv) an N protein from SARS-CoV-2, an S-RBD from SARS-CoV-2, an S protein from SARS-CoV-2, an S2 from SARS-CoV-2, an S protein from SARS-CoV, an S2 from SARS-CoV-2, an S protein from HCoV-OC43, an S protein from HCoV-HKU1, an S protein from HCoV-NL63, and an S protein from HCoV-229E;
(v) an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2;
or
(vi) an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2
is immobilized on a distinct binding domain on the surface; and (b) an ACE2 detection reagent.

A method for detecting multiple antibody biomarkers of interest in a sample, the method comprising: (a) contacting the biological sample with a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, and an HA from influenza A/Hong Kong H3;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, and an S protein from HCoV-229E;
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, and an S protein from HCoV-229E;
(iv) an N protein from SARS-CoV-2, an S-RBD from SARS-CoV-2, an S protein from SARS-CoV-2, an S2 from SARS-CoV-2, an S protein from SARS-CoV, an S2 from SARS-CoV-2, an S protein from HCoV-OC43, an S protein from HCoV-HKU1, an S protein from HCoV-NL63, and an S protein from HCoV-229E;
(v) an S protein from SARS-CoV-2, an S1 from HCoV-NL63, an N protein from SARS-CoV-2, an S1 from SARS-CoV, an S1 from SARS-CoV-2, an S1 from HCoV-HKU1, an S1 from HCoV-OC43, an S1 from HCoV-229E, and an S-RBD from SARS-CoV-2;
or
(vi) an S protein from SARS-CoV-2, an N protein from HCoV-NL63, an N protein from SARS-CoV-2, an N protein from SARS-CoV, an N protein from MERS-CoV, an N protein from HCoV-HKU1, an N protein from HCoV-OC43, an N protein from HCoV-229E, and an S-RBD from SARS-CoV-2
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with an ACE2 detection reagent; and (d) measuring concentration of the antibody biomarker in each binding complex.

A kit for detecting multiple antibody biomarkers of interest in a sample, the kit comprising, in one or more vials, containers, or compartments: (a) a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
or
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza/Shanghai H7, an HA from influenza B/Phuket, an HA from influenza B/Brisbane, and an F protein from RSV
is immobilized on a distinct binding domain on the surface; and (b) an ACE2 detection reagent.

A method for detecting multiple antibody biomarkers of interest in a sample, the method comprising: (a) contacting the biological sample with a surface comprising multiple binding domains, wherein each of:

(i) an N protein from SARS-CoV-2, an S-NTD from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
(ii) an N protein from SARS-CoV-2, an S-NTD from SARS-CoV-2, an S-RBD from SARS-CoV-2, an S protein from SARS-CoV-2, an S protein from SARS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza A/Shanghai H7, an HA from influenza B/Phuket, an HA from influenza B/Brisbane, and an F protein from RSV;
or
(iii) an N protein from SARS-CoV-2, an S-RBD from SARS-CoV-2, an S protein from SARS-CoV-2, an S protein from SARS-CoV, an S protein from MERS-CoV, an S protein from HCoV-OC43, an S protein from HCoV-HKU1, an S protein from HCoV-NL63, an S protein from HCoV-229E, an HA from influenza A/Hong Kong H3, an HA from influenza A/Michigan H1, an HA from influenza/Shanghai H7, an HA from influenza B/Phuket, an HA from influenza B/Brisbane, and an F protein from RSV
is immobilized on a distinct binding domain on the surface; (b) forming a binding complex in each binding domain, wherein the binding complex comprises the viral antigen and an antibody biomarker that binds to the viral antigen; (c) contacting the binding complex in each binding domain with an ACE2 detection reagent; and (d) measuring concentration of the antibody biomarker in each binding complex.

### EXAMPLES

### Example 1. Serology Study for SARS-CoV-2

Kits for sample collection that include a sample collection tube or vial, which optionally include a straw, are provided to subjects to self-collect saliva according to instructions provided in the kit. The collected samples are then sent to the laboratory, refrigerated upon storage, and processed and assayed within 24 hours of arrival.

Levels of salivary IgG. IgM, and IgA antibodies are measured using multiplexes of viral antigens coated on plates using a two-step immunoassay. The plate optionally includes an antigen for a prevalent influenza strain (H1 Michigan) as one positive control. An assay for total levels of IgA provides another control. The negative control is bovine serum albumin (BSA). Intra-well positive and negative controls are used to assess validity of individual samples. The multiplex of viral proteins is summarized in Table 4.

TABLE 4
Spike (S)-ECD (S1 + S2)
Spike (S)-S1 Domain
Spike (S)-S2 Fusion Domain
Spike (S)-RBD
Nucleocapsid (N)-Full Length
Membrane (M)-Full Length
Envelope (E)-Full Length
Positive Control (H1 Michigan)
Positive Control (Total IgA)
Negative Control (BSA)

Saliva samples (25 μL/well) are added to assay diluent (25 μL/well) preloaded into the well. Each sample is loaded in duplicate on three plates designated for measurements of IgG, IgM, and IgA antibodies (total volume requirement for saliva=300 μL). After incubation for 1 hour to allow salivary antibodies to bind the immobilized antigens, wells are aspirated to remove sample and washed three times. A secondary detection antibody specific for human IgG, IgM, or IgA is added to the corresponding plates. The secondary antibody is labeled with an electrochemiluminescence (ECL) probe. After incubation for 1 hour, wells are aspirated to remove unbound secondary antibody and washed three times. Read buffer are added, and the plates are read. The entire process requires 3 hours with 20 minutes of hands on-time for each plate.

Among participants that test positive for COVID-19, it is expected that a transient increase in IgM will occur within days of symptoms followed shortly after by IgG and IgA. Whereas IgM and IgA levels are expected to return to undetectable levels, IgG levels are expected to persist as an indicator of immunity. For most of the participants that test negative for COVID-19, undetectable levels of antibodies to SARS-CoV-2 antigen are expected at all time points. However, some of the participants that test negative for COVID-19 may be false negative or may become infected at later times, in which case antibodies to SARS-CoV-2 should be present.

For data analysis, cut-offs for reactivity to various antigens are established. Since infected individuals may be asymptomatic, archived saliva (n=100) collected prior to December 2019 are used as non-exposed controls. The cut-off is set as the maximum level measured in the non-exposed control plus 2.5 times the standard deviation of the maximum level.

### Example 2. Serology Assay for SARS-CoV-2 Using Peptide Antigens

Peptide antigens are designed to minimize cross-reactivity and improve specificity in the serology assay. Peptide sequences were selected from portions of viral proteins that are predicted to be immunogenic based on similarities with SARS-CoV and bioinformatics analysis. A subset is shown below in Table 5. Approximately 10% of these linear peptides are expected to cross-react with antibodies that recognize the epitope on the full-length protein that contains the peptide sequence.

TABLE 5
Nucleoptein (N)-C-Term IDR (1-49)
Nucleoprotein (N)-N-Term IDR (340-390)
Nucleoprotein (N)-Mid IDR (192-220)
Membrane (M)-Mid IDR (182-216)

The peptide antigens are utilized in serology assays described in Example 1. Based on the results, the serology panel described in Example 1 can be expanded to include one or more peptide antigens that show reactivity with anti-SARS-CoV-2 antibodies in saliva of COVID-19 patients.

### Example 3. Additional Serology Assays

Additional multiplex panels of viral proteins are shown below in Table 6. These viral proteins are utilized in serology assays described in Example 1.

TABLE 6

| Panel | Antigens |
|---|---|
| 1 | Spike constructs: S trimer, S1 subunit, S2 subunit, S-RBD, S N-terminal |
| (SARS-CoV- | domain Other proteins: Nucleocapsid, Envelope, Membrane proteins |
| 2 Panel) | |
| 2 | Spike (S) trimer from: HKU1, NL63, 229E, OC43, SARS |
| (Pan CoV | and MERS coronavirus strains |
| Panel) | |
| 2A | One or more of: SARS-CoV-2 S trimer, S1 subunit, S2 subunit, S-RBD, |
| | S N-terminal domain, Nucleocapsid, Envelope, Membrane proteins; and |
| | One or more of: S trimer from: HKU1, NL63, 229E, OC43, SARS |
| | and MERS coronavirus strains |
| 3 | Hemagglutinin (HA) trimers from influenza B, H1, H3 |
| (Respiratory | and H7 strains; Fusion (F) protein from RSV |
| Infection | |
| Panel) | |
| 3A | One or more of: SARS-CoV-2 S trimer, S1 subunit, S2 subunit, S-RBD, |
| | S N-terminal domain, Nucleocapsid, Envelope, Membrane proteins; and |
| | One or more of Hemagglutinin (HA) trimers from influenza B, H1, H3 |
| | and H7 strains; Fusion (F) protein from RSV |
| 4 | One or more of: SARS-CoV-2 S tritner, S1 subunit, S2 subunit, S-RBD, |
| | S N-terminal domain, Nucleocapsid, Envelope, Membrane proteins; |
| | One or more of: S trimer from: HKU1, NL63, 229E, OC43, SARS |
| | and MERS coronavirus strains; and One or more |
| | of Hemagglutinin (HA) trimers from influenza B, H1, H3 |
| | and H7 strains; Fusion (F) protein from RSV |
| 5 | SARS-CoV-2 N protein, SARS-CoV-2 S-NTD, SARS-CoV-2 S-RBD, |
| (SARS-COV- | SARS-CoV-2 S protein, SARS-CoV S protein |
| 2 Panel) | Detection: IgA, IgG, IgM |
| 6 | SARS-CoV-2 N protein, SARS-CoV-2 S-NTD, SARS-CoV-2 S-RBD, |
| (SARS-CoV-2 | SARS-CoV-2 S protein, SARS-CoV S protein |
| Neutralization | Detection: ACE2 |
| Panel) | |
| 7 | SARS-CoV-2 N protein, SARS-CoV-2 S-NTD, SARS-CoV-2 S-RBD, |
| (Coronavirus | SARS-CoV-2 S protein, SARS-CoV S protein, MERS-CoV S protein, |
| Panel I) | HCoV-OC43 S protein, HCoV-HKU-I S protein, influenza A H3 HA |
| | protein Detection: IgA, IgG, IgM |
| 8 | SARS-CoV-2 N protein, SARS-CoV-2 S-NTD, SARS-CoV-2 S-RBD, |
| (Respiratory | SARS-CoV-2 S protein, SARS-CoV S protein, MERS-CoV S protein, |
| Panel 1A) | HCoV-OC43 S protein, HCoV-HKU-1 S protein, influenza A H3 HA |
| | protein, influenza A H1 HA protein, influenza A H7 HA protein, |
| | influenza B/Phu HA protein, influenza B/Bris HA protein |
| | Detection: IgA, IgG, IgM. |
| 9 | SARS-CoV-2 N protein, SARS-CoV-2 S-NTD, SARS-CoV-2 S-RBD, |
| (Respiratory | SARS-CoV-2 S protein, SARS-CoV S protein. MERS-CoV S protein, |
| Panel 1B) | HCoV-OC43 S protein, HCoV-HKU-1 S protein, influenza A H3 HA |
| | protein, influenza A HH HA protein, influenza A H7 HA protein, |
| | influenza B/Phu HA protein, influenza B/Bris HA protein, RSV F protein |
| | Detection: IgA, IgG, IgM. |
| 10 | SARS-CoV-2 N protein, SARS-CoV-2 S-NTD, SARS-CoV-2 S-RBD, |
| (Coronavirus | SARS-CoV-2 S protein, SARS-CoV S protein, HCoV-OC43 S protein. |
| Panel 2) | HCoV-HKU-1 S protein, HCoV-NL63 S protein, HCoV-229E S protein |
| | Detection: IgA, IgG, IgM. |
| 11 | S.ARS-CoV-2 N protein, SARS-CoV-2 S-NTD, SARS-CoV-2 S-RBD, |
| (Respiratory | SARS-CoV-2 S protein, SARS-CoV S protein, HCoV-OC43 S protein, |
| Panel 2) | HCoV-HKU-1 S protein, HCoV-NL63 S protein, HCoV-229E S protein, |
| | influenza A H3 HA protein, influenza A H1 HA protein, influenza A H7 |
| | HA protein, influenza B/Phu HA protein, influenza B/Bris HA protein, |
| | RSV F protein Detection: IgA, IgG, IgM. |
| 12 | SARS-CoV-2 S protein, SARS-CoV-2 S-RBD, SARS-CoV-2 S-NTD, |
| (Pandemic | SARS-CoV-2 N protein, SARS-CoV S protein, MERS-CoV S protein |
| CoV Panel) | |
| 13 | FluB/Brisbane/60/2008 HA protein, FluB/Phuket/3073/2013 HA protein, |
| (Circulating | FluA/Michigan/45/2015 (H1N1) HA protein, |
| CoV and | FluA/HongKong/4801/2014 (H3N2) HA protein, MA/Shanghai/2/2013 |
| Flu Panel) | (H7N9) HA protein, HCoV-HKU1 S protein, HCoV-OC43 S protein |
| 14 | SARS-CoV-2 S protein, SARS-CoV-2 S-RBD, SARS-CoV-2 S-NTD, |
| (Mixed Panel) | SARS-CoV-2 N protein, SARS-CoV S protein, MERS-CoV S protein, |
| | HCoV-HKU1 S protein, HCoV-OC43 S protein, |
| | FluAlHongKong/4801/2014 (H3N2) HA protein |
| 15 | SARS-CoV-2 N protein, SARS-CoV-2 S-RBD, SARS-CoV-2 S protein, |
| (Coronavirus | SARS-CoV S protein, MERS-CoV S protein, HCoV-OC43 S protein, |
| Panel 3) | HCoV-HKU-1 S protein, HCoV-NL63 S protein, HCoV-229E S protein |
| | Detection: IgA, IgG, IgM. |
| 16 | SARS-CoV-2 N protein, SARS-CoV-2 S-RBD, SARS-CoV-2 S protein, |
| (Coronavirus/ | SARS-CoV S protein, MERS-CoV S protein, HCoV-OC43 S protein, |
| Respiratory | HCoV-HKU-1 S protein, HCoV-NL63 S protein, HCoV-229E S protein, |
| Panel | influenza A H3 HA protein, influenza A H1 HA protein, influenza A H7 |
| 3) | HA protein, influenza B/Phu HA protein, influenza B/Bris HA protein, |
| | RSV F protein Detection: IgA, IgG, IgM |
| 17 | SARS-CoV-2 N protein, SARS-CoV-2 S-RBD, SARS-CoV-2 S protein, |
| (Coronavirus | SARS-CoV S protein, SARS-CoV-2 S2, HCoV-OC43 S protein, |
| Panel 4) | HCoV-HKU-1 S protein, HCoV-NL63 S protein, HCoV-229E S protein |
| | Detection: IgA, IgG, IgM |

The SARS-CoV-2 panel (Panel 1) described in Table 6 is used for vaccine development and validation. Panel 1 includes major vaccine epitopes and other SARS-CoV-2 proteins that can discriminate the effects of a vaccine and/or infection. Panel 1 is used for diagnosis of SARS-CoV-2 and epidemiological studies to assess disease spread and immunity.

The Pan CoV and Respiratory Infection panels (Panels 2 and 3) described in Table 6 are used for vaccine development, e.g., for a vaccine against all coronaviruses. Panel 2 includes antigens from common coronavirus infections, and Panel 3 includes antigens from other respiratory viruses. Panels 2 and 3 are used for diagnostics and epidemiological studies, e.g., to distinguish the SARS-CoV-2 antibody response from those of other respiratory virus infections.

### Example 4A. Serology Assay for SARS-CoV-2 on Patient Samples

A serology assay to detect antibodies against SARS-CoV-2 in patient samples was performed using the following viral antigen panel; SARS-CoV-2 N protein, SARS-CoV-2 S protein, SARS-CoV-2 S-RBD, SARS-CoV-2 S-NTD, SARS-CoV S protein, MERS-CoV S protein, HCoV-HKU1 S protein, HCoV-OC43 S protein, FluB (Brisbane strain) HA protein, FluB (Phuket strain) HA protein, FluA H1 (Michigan strain) HA protein, FluA H3 (Hong Kong strain) HA protein, and FluA H7 (Shanghai strain) HA protein. Bovine serum albumin (BSA) was used as a negative control.

The antigens include SARS-CoV-2 recombinant antigens and SARS-CoV antigens, which were not expected to be cross-reactive with SARS-CoV-2 due to the low incidence of SARS in the patients whose samples were tested in this study. The viral antigens were immobilized in a 96-well plate or within an assay cartridge. The detection reagents were anti-IgG and anti-IgM antibodies labeled with an electrochemiluminescence (ECL) label, which allowed measurement of isotype-specific reactivity to the antigens. The tested samples were serum obtained from COVID-19 positive patients (n=13) and normal (non-COVID-19) patient serum (n=9) (all sera were diluted 500-fold for testing).

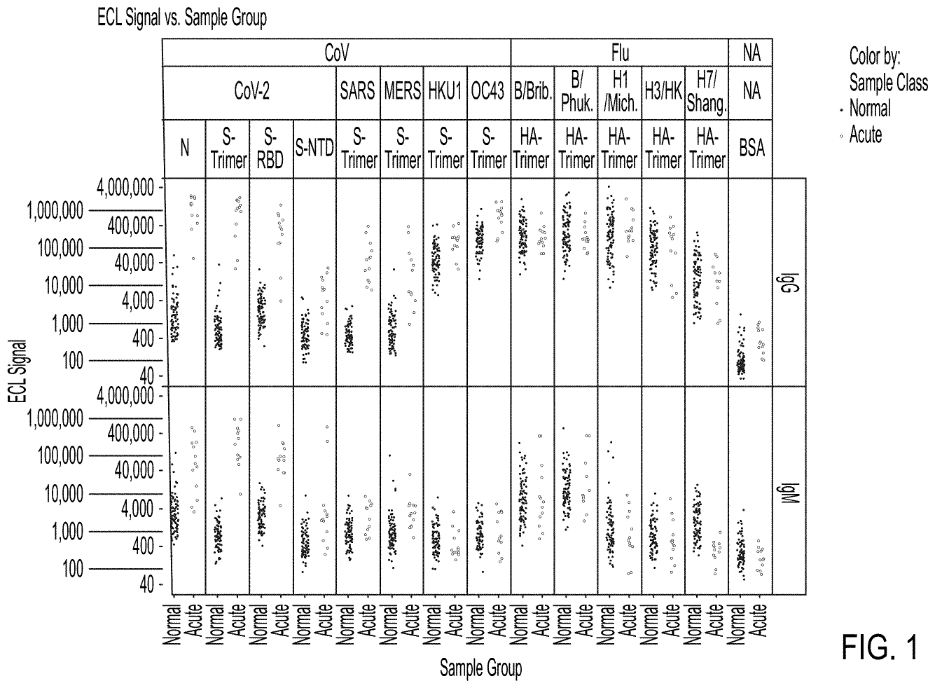

Results are shown in FIG. 1. IgG and IgM antibodies directed towards SARS-CoV-2 antigens were detected in COVID-19 patient serum samples, with minimal reactivity observed in normal serum samples. In contrast, immune reactivity to commonly circulating coronaviruses (HCoV-HKU1 and HCoV-OC43) and influenza viruses showed no separation between normal and COVID-19 patient samples, as expected.

### Example 4B. Serology Assay for SARS-CoV-2 with Monoclonal Antibody Sample

Performance of the serology assay was further evaluated using serial dilutions of a mixture of two humanized monoclonal antibodies: one with binding activity for SARS-CoV-2 N protein, and one with binding activity for the SARS-CoV-2 S protein or S-RBD. The viral antigen panel was the Pandemic CoV panel (panel 13) from Table 6.

Results in FIG. 29 shows the change in ECL signal for the different antigens in the panel. The ECL signal increased linearly for the targeted antigens, but remained at or near the baseline for the other antigens.

### Example 5A. Bridging Serology Assay for SARS-CoV-2 on Patient Samples

A bridging serology assay to detect SARS-CoV-2 antibodies was performed using the SARS-CoV-2 S-RBD antigen. The bridging serology assay used the simultaneous binding of antibodies to immobilized viral antigen and detection tag-labeled viral antigen, leading to a highly specific isotype-independent measurement of immune reactivity. The tested samples were from the same COVID-19 positive and normal patients as described in Example 4, except the samples were diluted 10-fold or 100-fold.

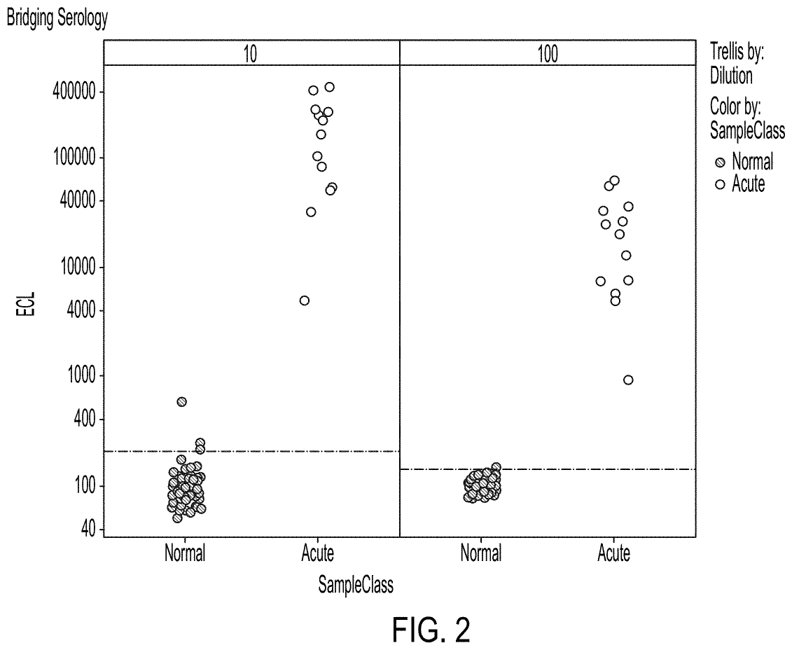

Results are shown in FIG. 2. For both 10-fold and 100-fold sample dilutions, clear separation of signal was observed between the COVID-19 patient and normal samples. High signals detected in the COVID-19 sera indicates the presence of anti-S-RBD antibodies.

### Example 5B. Bridging Serology Assay for SARS-CoV-2 on Patient Samples

Performance of the bridging serology assay described in Example 5A was further evaluated. FIG. 10 shows a titration curve in a bridging serology assay of a monoclonal antibody against SARS-CoV-2 S-RBD, confirming that presence of the antibody leads to formation of bridging complexes.

The tested patient samples in this Example included 96 negative samples, 54 “late positive” samples, and 57 “early positive” samples for SARS-CoV-2. Results are shown in FIG. 11, and the measured sensitivity and specificity (as point estimate and 95% confidence interval) are summarized in Table 7.

TABLE 7
Sensitivity and Specificity of Bridging Serology Assay

| | | |
|---|---|---|
| Specificity (Negative) | 99% | 95/96 samples |
| Sensitivity Late Positive) | 93% | 50/54 samples |
| Sensitivity (Early Positive) | 34% | 16/47 samples |

### Example 6A. Neutralization Serology Assay for SARS-CoV-2 on Patient Samples

As described herein, neutralization serology assays (also termed “competitive serology assay”) can allow assessment of the potential protective serological response present in the patient. A neutralization serology assay was performed to test SARS-CoV-2 antibody binding to immobilized SARS-CoV-2 S protein in the presence of the host cell protein receptor, ACE2, as competitor for the antigen. The ACE2 is labeled with a detection label. The tested samples were from the same COVID-19 positive and normal patients as described in Example 4, except the samples were diluted 10-fold or 100-fold.

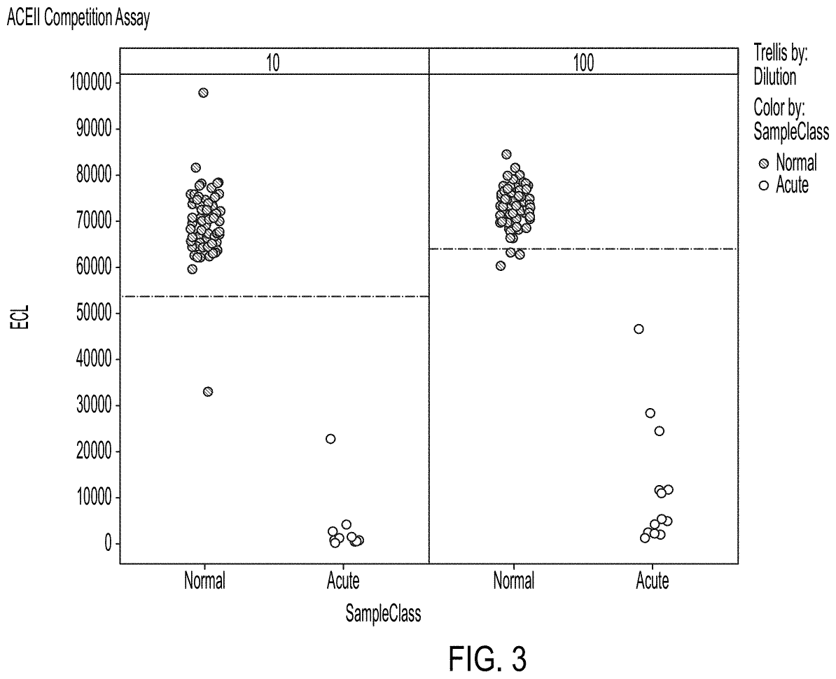

Results are shown in FIG. 3. For both the 10-fold and 100-fold sample dilutions, clear separation of signal was observed between the COVID-19 patients and normal samples. Lower signal detected in the COVID-19 sera indicates inhibition of the interaction between the SARS-CoV-2 S protein and its cognate receptor, ACE2, in COVID-19 patient samples.

### Example 6B. Neutralization Serology Assay for SARS-CoV-2 on Patient Samples

Performance of the neutralization serology assay described in Example 6A was further evaluated. FIG. 12 shows a titration curve in a neutralization serology assay of a monoclonal antibody that binds and inhibits the S protein from SARS-CoV and SARS-CoV-2. The titration curve indicates that the signal from labeled competitor, ACE2, decreased as expected when SARS-CoV S protein, SARS-CoV-2 S protein, or SARS-CoV-2 S-RBD were immobilized on the surface. However, the monoclonal antibody did not bind the S protein from HCoV-HKU1 or MERS-CoV (data not shown).

The tested patient samples in this Example are the same as in Example 5B. Results are shown in FIG. 13, and the measured sensitivity and specificity (as point estimate and 95% confidence interval) for immobilized SARS-CoV S protein, SARS-CoV-2 S protein, or SARS-CoV-2 S-RBD are summarized in Table 8.

TABLE 8
Sensitivity and Specificity of Neutralization Serology Assay

| | CoV-2 S protein | | COV S protein | | CoV-2 S-RBD | |
|---|---|---|---|---|---|---|
| Specialty | 98% | 94/96 | 99% | 95/96 | 97% | 93/96 |
| (Negative) | | samples | | samples | | samples |
| Sensitivity | 91% | 49/54 | 54% | 29/54 | 89% | 48/54 |
| (Late Positive) | | samples | | samples | | samples |
| Sensitivity | 28% | 13/47 | 13% | 6/47 | 28% | 13/47 |
| (Early Positive) | | samples | | samples | | samples |

### Example 6C. Neutralization Serology Assay for SARS-CoV-2 with Monoclonal Antibody Sample

Performance of the ACE2 neutralization serology assay was further evaluated using a serial dilution of a monoclonal antibody known to neutralize SARS-CoV. The viral antigen panel was the Mixed Panel (panel 15) from Table 6.

Results in FIG. 30 shows the change in ECL signal for the different antigens in the panel. At low antibody concentrations, labeled ACE2 bound to the antigens with an ACE2 receptor binding domain (SARS-CoV-2 S. SARS-CoV-2 S-RBD, and SARS-CoV S), which provided a high assay signal, but the ACE2 did not bind to antigens (including the S protein from other coronaviruses in the panel) that do not natively recognize ACE2. As the concentration of neutralizing antibody increased, the antibody bound to the target antigens and prevented their interaction with ACE2, causing a decrease in the assay signal.

### Example 6D. Neutralization Serology Assay for SARS-CoV-2 on Patient Samples

Performance of the neutralization serology assay described in Example 6A was further evaluated using samples from patients who had been tested to be negative, early positive, and late positive for COVID-19. FIG. 31 shows the results of a neutralization serology assay using the Mixed Panel (panel 15) from Table 6.

The labeled ACE2 competitor only generated significant assay signals on the antigens with known ACE2 binding activity (SARS-CoV-2 S and S-RBD, and SARS-CoV S). The results showed that the assay signals on the SARS-CoV-2 S and S-RBD antigens were significantly reduced (p<0.001) for the late infection group relative to the normal controls. The reduction in the geometric mean signal was 78-fold for S and 65-fold for S-RBD, and the ROC AUC values were 0.91 and 0.93, respectively. While FIG. 31 showed some visual evidence that some late infection subjects had some low levels of competitive cross-reactivity for the SARS-CoV S protein, the observed signal reduction was considerably less in magnitude than observed for the SARS-CoV-2 S protein.

### Example 6E. Neutralization Serology Assay for SARS-CoV-2 on Patient Samples

Performance of the neutralization serology assay described in Example 6A was further evaluated using serum samples that included 175 samples identified as negative for COVID-19 (collected prior to 2019); 40 samples from acute COVID-19 patients; and 76 samples from convalescent COVID-19 patients. Results are shown in FIG. 35. The left panel represents results obtained with a 1:10 sample dilution, and the right panel represents results obtained with a 1:100 sample dilution. The results show a clear separation between negative and convalescent samples, while acute samples showed some level of neutralizing antibodies, as expected. The specificity of the assay was 98.3%, and sensitivity was 94.7% for convalescent samples at 1:10 dilution.

### Example 7. Detection of SARS-CoV-2 Nucleocapsid Protein

A detection assay of the SARS-CoV-2 nucleocapsid (N) protein was performed with a radiation-inactivated SARS-CoV-2 viral preparation. Calibration curves using recombinant SARS-CoV-2 N protein were generated using two different detection assay formats: an immunoassay utilizing a detection reagent with a detectable label, comprising directly detecting the detectable label (Format 1); and an immunoassay utilizing a detection reagent with a nucleic acid probe, comprising extending the nucleic acid probe to form an extended sequence and contacting the extended sequence with a probe comprising a detectable label (Format 2).

The Format 1 assay was performed as follows: To each well of a 96-well plate containing immobilized anti-Nucleocapsid capture antibody, add 25 μL labeled anti-Nucleocapsid detection antibody labeled with SULFO-TAG™ ECL label, and 25 μL sample. Incubate for 1 hour at room temperature with shaking. Wash plate, add 150 μL ECL Read Buffer, and read plate. Total protocol time is approximately 1 hour.

The Format 2 assay was performed as follows: To each well of a 96-well plate containing immobilized anti-Nucleocapsid capture antibody, add 25 μL labeled anti-Nucleocapsid detection antibody labeled with nucleic acid probe, and 25 μL sample. Incubate for 1 hour at room temperature with shaking. Wash plate and add extension solution. Incubate for 15 minutes at room temperature with shaking. Wash plate and add detection solution. Incubate for 45 minutes at room temperature with shaking. Wash plate, add 150 μL ECL Read Buffer, and read plate. Total protocol time is approximately 2 hours.

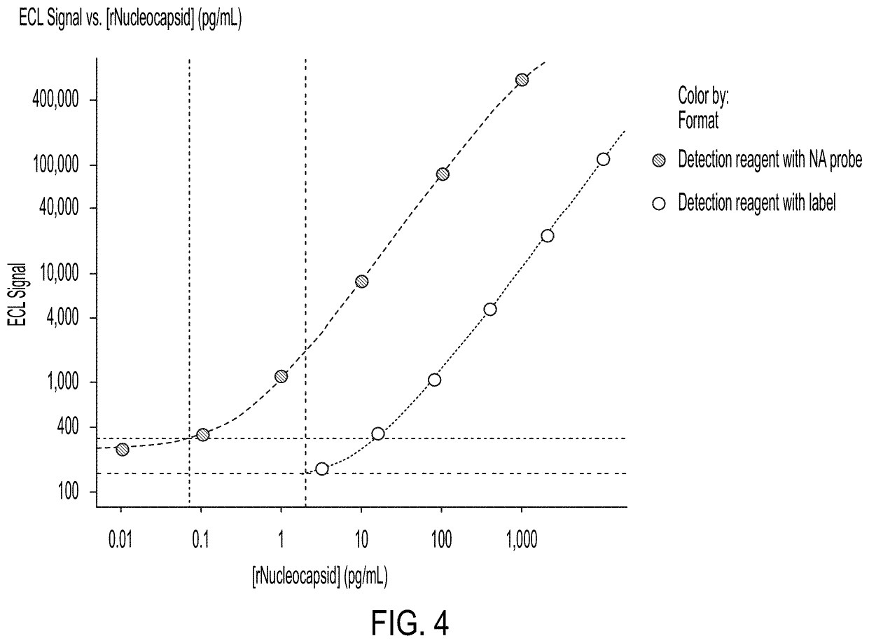

Results in FIG. 4 show the calibration curves of both formats, indicating that the “detection reagent with nucleic acid probe” format shows a 30-fold improvement in limit of detection compared with the “detection reagent with detectable label” format.

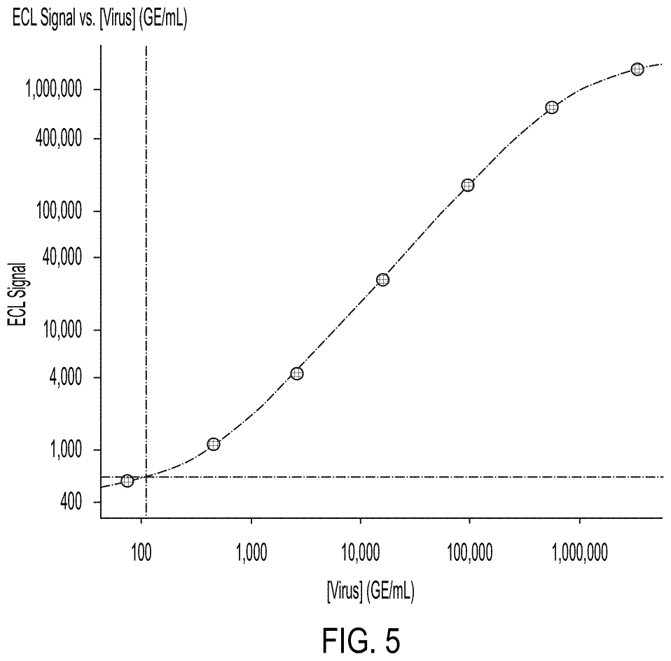

FIG. 5 shows a plot of the assay signal as a function of virus concentration, presented as genome equivalents per mL, in a radiation-inactivated SARS-CoV-2 viral preparation determined based on the calibration curves in FIG. 4. FIG. 5 shows a limit of detection of around 100 genomic equivalents per mL, demonstrating the capability to provide PCR-like performance in an immunoassay format without the problems of contamination and quantitation typically present in PCR assays.

### Example 8. Detection of SARS-CoV-2 Nucleocapsid and Spike Proteins in Clinical Samples

The two assay formats were used to test for SARS-CoV-2 N protein and SARS-CoV-2 S protein in extracted swab samples from two commercial vendors. The samples included 10 samples identified as negative and 30 samples identified as positive from PCR test.

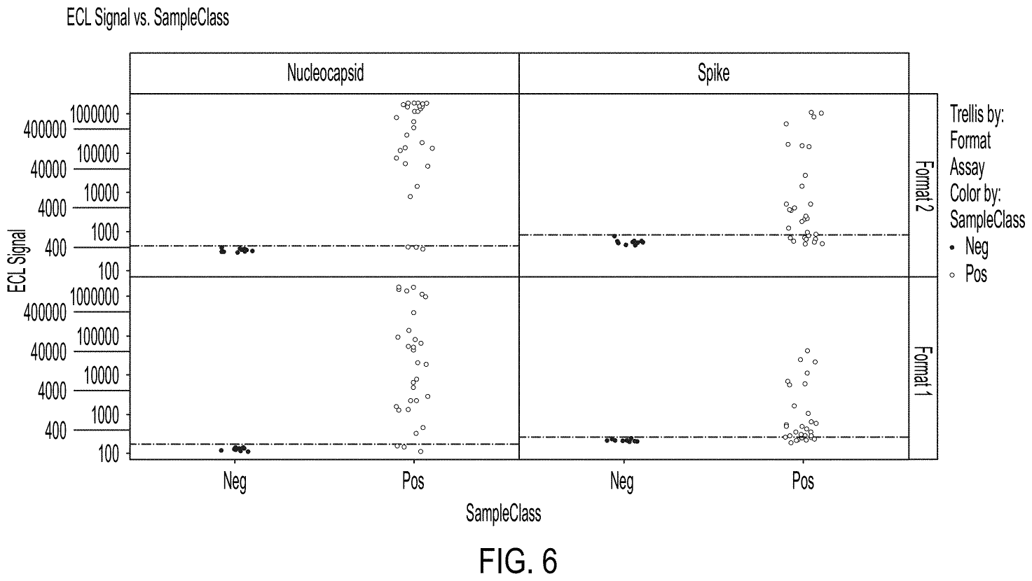

FIG. 6 shows the results from both assay formats. In the N protein assays, 27 of 30 putatively positive samples and 10 of 10 putatively negative samples were correctly identified. The point estimates and 95% confidence intervals for sensitivity and specificity were 90% (74%-99%) and 100% (69% to 100%), respectively. The true positive samples provided signals that ranged from 20-fold to 5000-fold above the assay threshold, thus demonstrating that the levels of protein in these swabs were easily detectable with the immunoassay. In the S protein assays, 22 of 30 putatively positive samples and 10 of 10 putatively negative samples were correctly identified, with sensitivity of 73% (54%-88%) and specificity of 100% (69%-100%).

### Example 9. SARS-CoV-2 Strain Typing Assay and SNP Detection

Multiplexed oligonucleotide ligation assays (OLA) were performed to detect the SARS-CoV-2 S and L strains, and to detect the SARS-CoV-2 S-D614 and S-D614G strains. A pair of targeting probes and a detection probe for each polymorphic site (locations 8782 and 28144 of the SARS-CoV-2 genome for S and L strain detection, and location 23403 of the SARS-CoV-2 genome for the S-D614 or S-D614G strain detection) were designed, to allow for single-base discrimination at the SNP site (3′ nucleotide of the targeting probe). The targeting probes have unique 5′ oligonucleotide tag sequences that are complementary to binding reagents on specific binding domains on a multi-well plate. The detection probes have a 5′ phosphate group for ligation and a 3′ biotin. OLA was used to ligate targeting and detection probes that aligned perfectly on the SARS-CoV-2 target nucleic acid. The ligated probes were then hybridized to the multi-well plate and detected by adding streptavidin-labeled SULFO-TAG™ and reading the signal with a plate reader.

Probes were designed against both the viral RNA strand and the cDNA strand that is synthesized upon reverse transcription. The assay was first performed with a synthetic DNA template that included the SNP sites and surrounding region (approximately 75 nucleotides). For viral RNA, one-step RT-PCR was performed to reverse transcribe and amplify the region surrounding each SNP site.

Several probe sets were tested to minimize off-target signal generation. Various ligation temperatures were also tested, as ligation temperature can also affect off-target signal generation. The probes that were determined to provide the best SNP determination are provided in Tables 10-12. The OLA conditions are provided in Table 9.

TABLE 9
OLA Cycling Conditions

| | | |
|---|---|---|
| 95° C. | 2 min | |
| 95° C. | 30 sec | × 30 cycles |
| 66° C. | 2 min | |
| 4° C. | Hold | |

TABLE 10
Probes

| Probe Name | Probe sequence |
|---|---|
| S 8782 | ACTGGTAACCCAGACATGATCGGTTATAACTA |
| Targeting probe | CCACCACGCTGA |
| | (SEQ ID NO: 1) |
| L 8782 | ACTCCCTGTGGGTGAGCTTAATGGTATAACTA |
| Targeting probr | CCACCACGCTGG |
| | (SEQ ID NO: 2) |
| 8782 | /5Phos/CTAAACCATGTGTCAAAATCAGCAT |
| Detection probe | G/3bio/ |
| | (SEQ ID NO: 5) |
| S 28144 | CATTTGGTCATTGGTTCAAGACGAGTTCCTGG |
| Targeting probe | CAATTAATTGTAAAAGGTG |
| | (SEQ ID NO: 3) |
| L 28144 | CTGGTCCGTTGTGGTCCTTCTAACGTTCCTGG |
| Targeting probe | CAATTAATTGTAAAAGGTA |
| | (SEQ ID NO: 4) |
| 28144 | /5Phos/AACAGGAAACTGTATAATTACCGAT |
| Detection probe | ATC/3Bio/ |
| | (SEQ ID NO: 6) |
| 23403A (S-D614) | CTAATAGCTCCTGTGCCCTCGTATGGGACTTC |
| Targeting probe | TGTGCAGTTAACA T |
| | (SEQ ID NO: 15) |
| 23403 (S-D614G) | AATCCGTCGACTAGCCTGAGAATTGGGACTTC |
| | TGTGCAGTTAACA C |
| | (SEQ ID NO: 16) |
| 23403 | /5Phos/CCTGATAAAGAACAGCAACCTGG/ |
| Detection probe | 3Bio/ |
| | (SEQ ID NO: 17) |

TABLE 11
Blocking Oligonucleotides

| Blocking | |
|---|---|
| Oligo Name | Blocking Oligonucleotide Sequence |
| 8782 Blocking | TATAACTACCACCACGCTGR |
| oligo 1 | (SEQ ID NO: 7) |
| 8782 Blocking | CTAAACCATGTGTCAAAATCAGCATG |
| oligo 2 | (SEQ ID NO: 8) |
| 28144 Blocking | GTTCCTGGCAATTAATTGTAAAAGGTR |
| oligo 1 | (SEQ ID NO: 9) |
| 28144 Blocking | AACAGGAAACTGTATAATTACCGATATC |
| oligo 2 | (SEQ ID NO: 10) |
| 23403 Blocking | GGGACTTCTGTGCAGTTAACA Y |
| oligo 1 | (SEQ ID NO: 18) |
| 23403 Blocking | CCTGATAAAGAACAGCAACCTGG |
| oligo 2 | (SEQ ID NO: 19) |

TABLE 12
Primers

| Prhner Name | Primer sequence |
|---|---|
| 8782 Froward primer | aggctattgatggtggtgtca |
| | (SEQ ID NO: 11) |
| 8782 Reverse primer | gcacgacaaaacccacttct |
| | (SEQ ID NO: 12) |
| 28144 Forward primer | cgtggatgaggctggttcta |
| | (SEQ ID NO: 13) |
| 28144 Reverse primer | tttcgctgattttggggtcc |
| | (SEQ ID NO: 14) |
| 23403 Forward primer | acactactgatgctgtccgt |
| | (SEQ ID NO: 20) |
| 23403 Reverse primer | gcccctattaaacagcctgc |
| | (SEQ ID NO: 21) |

The probes and assay conditions were tested with the following SARS-CoV-2 isolate: catalog number NR-52285 (deposited by the Centers for Disease Control and Prevention and obtained through BEI Resources, NIAID, NIH: Genomic RNA from SARS-Related Coronavirus 2, Isolate USA-WA1/2020). Based on analysis of this sequence deposited in NCBI for this isolate (GenBank ID: MN985325.1), this isolate should be categorized as S strain. Upon analysis of this isolate with the OLA and detection assay described in this Example, the genotype was confirmed to be the S strain, i.e., 8782T and 28144C. This isolate was also expected to be categorized as the S-D614 strain. The OLA and detection assay in this Example also correctly identified the genotype as the S-D614 strain, i.e., 23403A.

### Example 10. One-Step RT-PCR for Amplifying Viral Nucleic Acid

An exemplary protocol for reverse transcribing and amplifying a viral RNA (e.g., SARS-CoV-2 RNA) is described in this Example.

1) Extract RNA from a sample containing the virus of interest, e.g., SARS-CoV-2;

2) Prepare a Master Mix containing a single primer pair or multiplexed primer pairs and a one-step RT-PCR mix (e.g., from a commercially available source);

3) Add the sample RNA to the Master Mix:

4) Program a thermocycler to run the following steps (Table 13):

TABLE 13
Thermocycler Conditions

| Step | Cycles | Temp | Time |
|---|---|---|---|
| UNG incubation | 1 | 25° C. | 2 min |
| RT incubation | 1 | 50° C. | 15 min |
| Polymerase activation | 1 | 95° C. | 2 min |
| Amplification | 45 | 95° C. | 3 sec |
| | | 55° C. | 30 min |
| Hold | 1 | 4° C. | Hold |

The number of amplification cycles and annealing temperature can be adjusted based on the experiment (e.g., length of nucleic acid to be amplified).

The PCR product is ready to be tested with the nucleic acid detection methods described herein.

### Example 11. Serology Assay for SARS-CoV-2 on Patient Samples

A further serology assay to detect antibodies against SARS-CoV-2 in patient samples was performed using the following two viral antigen panels. Panel 1 included the SARS-CoV-2 N protein, SARS-CoV-2 S protein, SARS-CoV-2 S-RBD, SARS-CoV-2 S-NTD, SARS-CoV S protein, and MERS-CoV S protein. Bovine serum albumin (BSA) was used as a negative control. Panel 2 included the HCoV-HKU1 S protein, HCoV-OC43 S protein, FluA H1 (Michigan strain) HA protein, FluA H3 (Hong Kong strain) HA protein, FluA H7 (Shanghai strain) HA protein, FluB (Brisbane strain) HA protein, and FluB (Phuket strain) HA protein. Detection was performed using an anti-IgG antibody, in a similar manner as described above in Example 4.

The patient samples (N=99) were divided into three main classes: negative (“normal” sera collected pre-emergence of SARS-CoV-2); early infection (sera collected ≤7 days from a positive PCR test result; N=47); and late infection or convalescent (sera collected >7 days from a positive PCR test result; N=54). Patient samples were diluted 1:5000.

Results are shown in FIGS. 16A (Panel 1, IgG), 16B (Panel 2, IgG), and 34 (combined results for Panels 1 and 2, IgM). The viral antigens in Panel 1 showed a difference in IgG levels (based on ECL signal) between normal and positive patient samples, while the viral antigens in Panel 2 and the BSA negative control showed similar IgG levels across all three patient sample classes.

Serology assays were also performed using 1:500 dilution of patient samples and tested for IgG against SARS-CoV-2 S protein and SARS-CoV-2 N proteins. The assay cut-off thresholds were determined to maximize sensitivity and specificity for each antigen (only compared late infection and normal samples when setting the signal threshold). Assay results were based on a combined analysis, in which a sample is only deemed positive if IgG against both S protein and N protein were detected.

Results are shown in FIG. 17, with the sensitivity and specificity listed in Table 14.

TABLE 14
Serology Assay Sensitivity and Specificity

| | N protein | | S protein | | Combined | |
|---|---|---|---|---|---|---|
| Positives | Sens | Spec | Sens | Spec | Sens | Spec |
| Late Infection | 93% | 94% | 94% | 97% | 89% | 100% |
| Early Infection | 40% | 94% | 30% | 97% | 78% | 100% |
| All | 67% | 94% | 64% | 97% | 60% | 100% |

A further combined analysis of measured IgG levels against the SARS-CoV-2 S protein and S-RBD was performed. The combined sensitivity for early infection was 23%; the combined sensitivity for late infection was 91%, and the combined specificity was 99%.

### Example 12. Correlation of Different Serology Assay Formats

Results of the bridging serology assay in Example 5B and neutralization serology assay in Example 6B were plotted against results of the classical serology assay in Example 11 to determine the correlation between the different serology assay formats. The plot is shown in FIG. 18, indicating the results between different assay formats are well-correlated.

FIG. 32A shows the correlation of the indirect serology assays for IgG against SARS-CoV-2 S with four other serology assays (IgG against SARS-CoV-2 N, IgG against SARS-CoV-2 S-RBD. IgM against SARS-CoV-2 S, and ACE2 competitor assay). Elevated levels of IgG against SARS-CoV-2 S was associated with elevated levels of IgG against SARS-CoV-2 N (r²=0.86) and RBD (r²=0.87). Correlation was only observed if samples from infected patients were included in the analysis. Association between the assay signals was weak if the analysis was limited to just the naïve samples, in which case the r² values dropped to 0.07 and 0.17, respectively. A similar result was obtained by comparing the measured IgG and IgM response to SARS-CoV-2 S, which provided an r² value of 0.88 for the full sample set and 0.00 when limited to the naïve samples. Finally, generation of IgG antibodies that could bind SARS-CoV-2 S as measured by the indirect format was generally associated with the production of antibodies targeting the ACE2 binding site as measured by the ACE2 competition format (r²=0.87).

The weak correlation of signals for naïve samples provides a potential approach to improving assay performance by combining the results from multiple assays. For example, in the correlation plot for IgG against N vs. IgG against S (top left panel of FIG. 32A), there were some naïve samples that provided signals above the selected threshold for N but not S (top left area of panel) or S but not N (bottom right are of panel). The classification accuracy was further evaluated by requiring two assays to call a sample positive, as a method for reducing the impact of false positive signals for individual assays. FIG. 32B shows the classification performance for the possible pairings of the assays shown in FIG. 32A. In all four cases shown in the table in FIG. 32B, the combined assay result improved the specificity for classification of the naïve samples to 99% or 100% (0 or 1 false positive out of 95 samples). The improvement in specificity was accompanied by a small decrease in sensitivity: the sensitivities for classifying the late infection samples ranged from 89% to 91% for the two-assay combination compared to a range of 91% to 94% for the individual assays. In particular, combining the measurements of the IgG response to SARS-CoV-2 N and S proteins provided sensitivities of 28% and 89% for classifying the early and late infection samples, respectively, and a specificity of 100% for classifying the naïve samples.

### Example 13. SARS-CoV-2 Single Nucleotide Polymorphism (SNP) Assay

Ten (10) nasal swabs from COVID-19-positive patients and S-type reference material were tested with the oligonucleotide ligation assay (OLA) to detect the SARS-CoV-2 S and L strains at polymorphic sites 8782 and 28144, as described in Example 9.

Results are shown in FIGS. 19A-19D. An initial screening experiment using synthetic template DNA, based on the SARS-CoV-2 genome sequence, showed that SNPs at locations 8782 (T or C) and 28144 (C or T) were correctly identified using probes designed against the viral sequence (not cDNA) and a ligation temperature of 66° C. (FIG. 19A). The 8782T variant (FIG. 19B) and the 28144C variant (FIG. 19C), both associated with the S-strain, were identified in patient sample 2 and the S-strain control sample, while the remaining patient samples were identified to contain the L-strain. FIG. 19D summarizes the data from FIGS. 19B and 19C and shows the allele frequency from the sample set.

### Example 14. Serology and Host Biomarker Assays for SARS-CoV-2 Patient Samples

Serology assays and immunoassay for host biomarkers were performed on various patient sample sets: Sample Set 1A; plasma from six patients who had been hospitalized with COVID-19, with varying levels of RNAemia (ranging from undetectable to ˜1000 genomes/mL); Sample Set 1B: plasma from patients who were negative for COVID-19 from the same clinical source as Sample Set 1A; Sample Set 2A: twenty serum samples from patients known to be positive for COVID-19; Sample Set 2B: twenty plasma from samples from patients known to be positive for COVID-19; Sample Set 3 (control) included 28 plasma samples known to be negative for COVID-19 (obtained before emergence of COVID-19).

Sample Sets 1A, 1B, 2A, 2B, and 3 (serum/plasma samples) were tested with the following panels:

| Panel | Analyte (Host Biomarker or Antibody) |
|---|---|
| Proinflammatory | IFN-γ, IL-1β, 1L-2, IL-4, IL-6, IL-8, |
| Biomarkers Panel | IL-10, IL-12p70, IL-13, TNF-α |
| Chemokine | Eotaxin, Eotaxin-3, IL-8, |
| Biomarkers Panel | IL-8 (HA), IP-10, MCP-1, MCP-4, |
| | MDC, MIP-1α, MIP-1β, and TARC |
| Vascular Injury | CRP, ICAM-1, SAA, and VCAM-1 |
| Biomarkers Panel | |
| Angiogenesis | FGF (basic) PIGF, Tie-2, VEGF-A, |
| Biomarkers Panel | VEGF-C, VEGF-D, VEGFR-1/Fit-1 |
| Serology Panel | Binding: SARS-CoV-2 N protein, |
| (Coronavirus Panel 2 | SARS-CoV-2 S-NTD, |
| from Example 3) | SARS-CoV-2 S-RBD, |
| | SARS-CoV-2 S protein, |
| | SARS-CoV S protein, |
| | HCoV-OC43 S protein, |
| | HCoV-HKU-1 S protein, |
| | HCoV-NL63 S protein, |
| | HCoV-229E S protein, |
| | BSA (control) |
| | Detection: IgA, IgG, IgM |

Results for Sample Sets 1A, 1B, 2A, 2B, and 3 tested with the Serology Panel are shown in FIG. 20A. The Serology Panel results showed a clear distinction between Sample Sets 1A, 2A, and 2B (COVID-19 positive) from Sample Sets 1 B and 3 (COVID-19 negative), as indicated by high antibody binding to SARS-CoV-2 antigens.

Results for Sample Sets 1A, 1B, 2A, 2B, and 3 tested with the Proinflammatory Panel are shown in FIGS. 20B (measured concentration) and 20C (measured ECL signal). The results showed that levels of IL-2, IL-4, and IL-13 were close to background for all tested samples. Levels of IL-6, IL-8, IFN-γ, and IL-10 were high in positive plasma Sample Set 1A. Levels of IL-10 were high in the positive plasma Sample Sets 1A and 2A.

Results for Sample Sets 1A, 1B, 2A, 2B, and 3 tested with the Chemokine Panel are shown in FIGS. 20D (measured concentration) and 20E (measured ECL signal). The results showed that levels of IL-8 and Eotaxin-3 were close to background for all tested samples. The difference in the IL-8 results for the Chemokine Panel and Proinflammatory Panel may be attributed to assay-specific parameters. Levels of MIP-1a, MIP-1b, and IP-10 were high in positive plasma Sample Set 1A. Levels of MCP-1, TARC, and MCP-4 were high in all positive Sample Sets 1A, 2A, and 2B.

Results for Sample Sets 1A, 1B, 2A, 2B, and 3 tested with the Vascular Injury Panel are shown in FIGS. 20F (measured concentration) and 20G (measured ECL signal). The results showed that levels of CRP and SAA were very high (>100 μg/mL) in positive Sample Set 1A, and the measured ECL signal was very close to the upper limit of detection (1×10⁶ counts). Levels of ICAM-1 and VCAM-1 generally correlated with the CRP and SAA levels.

Results for Sample Sets 1A, 1B, 2A, 2B, and 3 tested with the Angiogenesis Panel are shown in FIGS. 20H (measured concentration) and 20I (measured ECL signal). The results showed that levels of Flt-1 and PIGF were high in positive plasma Sample Set 1A. Levels of bFGF were high in the positive plasma Sample Sets 1A and 2A. Levels of VEGF-A and VEGF-C were high all positive Sample Sets 1A, 2A, and 2B. Levels of Tie-2 was similar across all tested samples.

The ECL signals for the host biomarkers as described above were then correlated with the ECL signal from antibodies against SARS-CoV-2, as measured with the Serology Panel.

Results for the correlation of the Proinflammatory Panel biomarkers with antibodies against SARS-CoV-2 S protein (“anti-S-antibody”) are shown in FIG. 21A. The results showed that the correlations for most biomarkers were driven by positive Sample Set 1A. Of the proinflammatory biomarkers, IL-1β, which had high levels in positive Sample Sets 1A and 2A, had the strongest correlation with anti-S-antibody levels.

Results for the correlation of the Chemokine Panel biomarkers with anti-S-antibody are shown in FIG. 21B. The results showed that MCP-1, MCP-4, and TARC levels had good correlation with anti-S-antibody levels.

Results for the correlation of the Vascular Injury Panel with anti-S-antibody are shown in FIG. 21C. The results showed generally low correlation between the vascular injury biomarkers and anti-S-antibody.

Results for the correlation of the Angiogenesis Panel with anti-S-antibody are shown in FIG. 21D. The results showed that VEGF-A and VEGF-C levels had good correlation with anti-S-antibody levels. The other biomarker correlations were mainly driven by positive Sample Set 1A and were therefore weaker.

### Example 15. EV Assays for SARS-CoV-2 Patient Samples

Immunoassays for isolating and detecting EVs were performed with an EV sample, which included EVs purified by an IZON SEC column from the plasma of Sample Sets 1A (COVID-19 positive) and 1B (COVID-19 negative) in Example 14. Sample Set 1A contained Sample IDs 7-12, and Sample Set 1B contained Sample IDs 1-6. The EV samples were tested with the following panels:

| Panel | Binding Reagent Target |
|---|---|
| TSPAN Panel | CD81, CD9, CD63, IgG (control) |
| Spike/TSPAN Panel | SARS-CoV-2 S protein, IgG (control) |

The TSPAN Panel used binding and detection reagents that bind to common EV markers, known as tetraspanins (“TSPAN”). IgG was used as a binding reagent negative control known not to bind EVs. Results of the TSPAN Panel assay are shown in FIG. 22A. The results showed that both COVID-19 positive and negative samples, as well as the “Expi” positive EV control sample, all contained EVs with the common tetraspanin markers CD81, CD9, and CD63. Comparison between undiluted (“neat”) and 10-fold diluted samples showed that the ECL signals were also diluted linearly. There is no general trend in ECL signal or tetraspanin heterogeneity between COVID-19 positive and negative samples.

The Spike/TSPAN Panel used a binding reagent that binds to the SARS-CoV-2 S protein, and detection reagents that bind to tetraspanins. IgG was used as a binding reagent negative control. Results of the Spike/TSPAN Panel are shown in FIG. 22B. The results showed that only two samples, Sample ID 8 and Sample ID 10, displayed higher ECL signal when captured with the SARS-CoV-2 S protein binding reagent as compared to IgG control binding reagent.

The EV samples were then tested for the presence of SARS-CoV-2 spike (S) and nucleoprotein (N). Each sample was subjected to assay buffer with or without TRITON™ X-100, which is capable of lysing the EVs and allows for assessment of EV internal components. Results are shown in FIG. 23. Sample ID 6, which was labeled by the clinical source as negative for COVID-19, nonetheless showed ECL signal for S protein, and was determined as likely mislabeled by the sample provider. Results also showed that TRITON™ X-100 had little effect on the S or N protein ECL signals, indicating that it is unlikely that SARS-CoV-2 proteins are associated with EVs, or that the concentration of TRITON™ X-100 used was not sufficient to lyse the EVs. A further explanation is that the S and N proteins can be present in immune complexes that co-eluted with EVs during SEC purification, suggesting a possible method for isolating immune complexes.

### Example 16. Uniformity Testing of Immobilized Antigens for Serology Assay

96-well assay plates with immobilized viral antigens was tested for uniformity (i.e., quantity and reproducibility) of antigen immobilization. The viral antigens contained a 6×His tag (SEQ ID NO: 547). An anti-His6 antibody was labeled with an electrochemiluminescent (ECL) label using known methods and diluted in an assay diluent. The assay plates were blocked with blocking solution and washed with wash buffer. A 50 μL volume containing 3 μg/mL of ECL-labeled anti-His6 antibody was added to each well of the plate, and the plates were incubated for 2 hours with shaking. After washing, 150 μL of an ECL read buffer was added to each well, and the plate was read on an MSD SECTOR® 6000 plate reader.

The uniformity test described above was performed on a three-panel plate having wells coated with three different viral antigen panels: panel 1: SARS-CoV-2 S protein, N protein, S-NTD, and S-RBD, SARS-CoV S protein, and MERS-CoV S protein; panel 2: HCoV-HKU1 S protein. HCoV-OC43 S protein, FluA H7(Shanghai) HA protein, FluA H1(Michigan) HA protein, FluA H3(Hong Kong) HA protein, FluB(Bris) HA protein, and FluB(Phuket) HA protein; panel 3: SARS-CoV-2 S protein, N protein, S-NTD, and S-RBD, SARS-CoV S protein, MERS-CoV S protein, HCoV-HKU1 S protein, HCoV-OC43 S protein, and FluA H3(Hong Kong) HA protein.

Results for the three-paneled plate are shown in FIG. 24A. The average signals for the different His6 (SEQ ID NO: 547)-containing viral antigens were roughly 1000 to 10000-fold above the signal for a non-His6 (SEQ ID NO: 547) control array element (BSA), demonstrating efficient and comparable immobilization of all the antigens. The average intra-plate coefficient of variation (CV) was generally less than 10% for all viral antigens. Binding of labeled monoclonal antibodies against the individual protein antigens was used to confirm that the antigens were immobilized in the correct binding domains and were recognized by the appropriate antibodies, as shown in FIG. 24B.

A further uniformity test was performed on the three-panel plate described above, using a blend of serum samples from subjects known to be COVID-19 positive. Results for the three-paneled plate are shown in FIG. 25. Similar to the His6 (SEQ ID NO: 547) uniformity test, the average intra-plate coefficient of variation (CV) was generally less than 10% for all viral antigens, indicating the viral antigens were uniformly immobilized on the plate.

### Example 17. Uniformity Testing of Immobilized Antigens for Serology Assay—Multiple Lots

The uniformity test using anti-His antibody described in Example 16 was also performed for three different lots of plates coated with SARS-CoV-2 S protein, N protein, and S-RBD. Results are shown in FIG. 26. The average intra-plate coefficient of variation (CV) and the average inter-plate CV were both less than 10%, indicating the viral antigens were uniformly immobilized on the plates from all three lots.

The three lots of plates were also tested to confirm that the viral antigens were immobilized at the correct binding domains in the wells. Labeled monoclonal antibody against SARS-CoV-2 S protein was added at 10 ng/mL to evaluate immobilization of SARS-CoV-2 S protein and S-RBD; labeled monoclonal antibody against SARS-CoV-2 N protein was added at 1 ng/mL to evaluate immobilization of SARS-CoV-N protein. Results are shown in FIGS. 27A (specific ECL signal) and 27B (% specific binding). All binding domains had the expected reactivity with its corresponding antibody, as shown in the shaded areas. The plates from all three lots had comparable signals from the anti-N protein antibody on the binding domains immobilized with SARS-CoV-2 N protein. The plates from all three lots also had comparable signals from the anti-S protein antibody on the binding domains immobilized with SARS-CoV-2 S protein and S-RBD. The non-specific binding percentage was less than 0.24% for all the plate lots. Correlations between plate lots of the signals for each of the immobilized antigens are shown in FIGS. 28A-C (FIG. 28A: N protein; FIG. 28B: S protein; FIG. 28C: S-RBD). The R-value for all immobilized antigens across all three lots were greater than 0.99, indicating high correlation and uniformity of immobilization.

### Example 18. Evaluation of Assay Performance

The sensitivity and specificity of serology assays were determined for IgG and IgM using SARS-CoV-2 S, S-RBD, and N antigens (see Example 4B), and ACE2 competitor (neutralization) serology assays using SARS-CoV-2 S and S-RBD antigens (see Example 6D). ROC analysis was used to identify the threshold for each combination of format and antigen that maximized the sum of the specificity and sensitivity for separating the late infections from the normal controls. This threshold was then applied to both the early infection and late infection data sets.

Using the optimal thresholds, the different assays shown in FIG. 33 provided similar sensitivity and specificity values, with largely overlapping 95% confidence limits. All the assays provided point estimates for specificity that were greater than or equal to 95%, except for the measurement of IgM against SARS-CoV-2 S protein. Measurements of IgG using the indirect serology format provided the highest point estimates for sensitivity in classifying late infections (point estimates of sensitivity for S, RBD and N ranged from 93% to 94%). Point estimates for measurement of IgM using the indirect serology format or inhibitory antibodies using the ACE2 competition format were slightly lower ranging from 85% to 91%, with the best sensitivity in both formats provided by the SARS-CoV-2 S antigen (91% sensitivity in both the IgM and ACE2 formats). All the assays provided lower sensitivity for measuring early infection with point estimates for sensitivity ranging from 26% to 47%. There was no evidence that measurement of IgM provided statistically higher sensitivity for classification of the early infection samples, relative to measurement of IgG.

### Example 19. Protocol for Serology Assay

A serology assay provided herein measures IgG and IgM from human subjects to the following viral antigens from SARS-CoV-2: S, N, and S-RBD. Each of the viral antigens is immobilized on a distinct binding domain in a well of a 384-well assay plate, wherein the well contains four distinct binding domains. The fourth binding domain comprises an immobilized control protein, BSA, that does not bind human IgG or IgM. Antibodies (e.g., IgG and/or IgM), if present in the sample, bind to the viral antigens immobilized on the plate. Anti-human IgG or IgM detection antibodies conjugated with an ECL label are added, which bind to any antigen-bound antibodies. The ECL label is measured, and the intensity of the ECL signal is proportional to the amount of antibody present in the sample, thereby providing a quantitative measurement of antibodies in the sample that are specific to the different viral antigens.

An exemplary protocol for the serology assay can be conducted as follows:

Step 1:

Prepare assay plate. Add 50 μL/well of blocking solution to the plate. Seal the plate with an adhesive plate seal and incubate at room temperature without shaking for 1 hour. In the meantime, prepare calibrators, controls, and samples.

Calibrator preparation. The calibration reagents contain known quantities of IgG and IgM. Prepare a 7-point calibration curve, e.g., with 4-fold serial dilutions from a provided stock calibration reagent and diluent. The diluent solution is used as the “blank” calibrator.

Control preparation. Prepare three levels of controls, each containing a known concentration (e.g., in AU/mL) of human IgG and IgM to the three SARS-CoV-2 antigens (S, N, and S-RBD).

Sample preparation. Prepare the sample(s) by diluting with the diluent. Samples should be diluted between 500-fold and 5000-fold.

Step 2: Calibrators, controls, and sample addition. After incubating the plate with the blocking solution, wash the plate 3 times with at least 90 μL/well of wash buffer. Add 25 μL of diluted samples, calibrators, and controls to their designated wells in the plate. Seal the plate with an adhesive plate seal and incubate at room temperature with shaking (˜15100 rpm) for 4 hours.

In the meantime, prepare detection antibody solution by diluting the stock detection antibody solution to its specified working concentration.

Step 3: Detection antibody addition. After the sample incubation, wash the plate 3 times with at least 90 μL/well of wash buffer. Add 25 μL/well of the prepared detection antibody solution to the plate. Seal the plate with an adhesive plate seal and incubate at room temperature with shaking (˜1500 rpm) for 1 hour.

Step 4. Read buffer addition. After the detection antibody incubation, wash the plate 3 times with at least 90 μL/well of wash buffer. Add 40 μL/well of read buffer to the plate, and immediately following the addition, read the plate on an appropriate assay instrument.

### Example 20. Protocol for ACE2 Competitor Assay

An ACE2 competitive assay (also referred to as “neutralization assay”) provided herein measures the activity of circulating serum antibodies in human subjects (“blocking antibodies”) that block the binding of ACE2 to its cognate binding partner, e.g., the following viral antigens from SARS-CoV-2: S, N, and S-RBD. Each of the viral antigens is immobilized on a distinct binding domain in a well of a 384-well assay plate, wherein the well contains four distinct binding domains. The fourth binding domain comprises an immobilized control reagent, BSA, that does not bind human antibody. Blocking antibodies, if present, bind to the immobilized viral antigens. Human ACE2 protein conjugated with an ECL label is added, and in the absence of blocking antibodies in the sample, the labeled ACE2 binds to its binding partner on the plate. Elevated concentrations of blocking antibodies proportionally decrease the amount of ACE2 that binds. The ECL label is measured, and the intensity of the ECL signal is inversely proportional to the amount of antibody present in the sample, thereby providing a quantitative measurement of blocking antibody in the sample.

An exemplary protocol for the serology assay can be conducted as follows:

Step 1:

Prepare assay plate. Add 50 μL/well of blocking solution to the plate. Seal the plate with an adhesive plate seal and incubate at room temperature without shaking for 1 hour. In the meantime, prepare calibrators and samples.

Calibrator preparation. The calibration reagents contain known quantities of IgG and IgM. Prepare a 7-point calibration curve, e.g., with 4-fold serial dilutions from a provided stock calibration reagent and diluent. The diluent solution is used as the “blank” calibrator.

Sample preparation. Prepare the sample(s) by diluting with the diluent. Samples should be diluted between 10-fold and 100-fold.

Step 2: Calibrators, controls, and sample addition. After incubating the plate with the blocking solution, wash the plate 3 times with at least 90 μL/well of wash buffer. Add 15 μL of diluted samples, calibrators, and controls to their designated wells in the plate. Seal the plate with an adhesive plate seal and incubate at room temperature with shaking (˜1500 rpm) for 4 hours.

In the meantime, prepare ACE2 detection solution by diluting the stock ACE2 detection solution to its specified working concentration.

Step 3: ACE2 addition. After the sample incubation, wash the plate 3 times with at least 90 μL/well of wash buffer. Add 15 μL/well of the prepared ACE2 detection solution to the plate. Seal the plate with an adhesive plate seal and incubate at room temperature with shaking (˜1500 rpm) for 1 hour.

Step 4. Read buffer addition. After the detection antibody incubation, wash the plate 3 times with at least 90 μL/well of wash buffer. Add 40 μL/well of read buffer to the plate, and immediately following the addition, read the plate on an appropriate assay instrument.

### Example 21. Protocol for Two-Layer Pooling Strategy

An exemplary approach for performing a two-layered pooling strategy in a 96-well plate is illustrated in FIG. 38. Individual samples are provided in 8×10 blocks of wells (e.g., columns 3 to 12 of a 96-well plate). The first layer comprises a pooled sample size of 80. The second layer comprises a pooled sample size of 10. The following protocol is used:

1. Transfer samples to individual sample plates (Plate Format 1 (PF1) plates shown in FIG. 38). The individual samples are contained in the 80 wells from rows A-H and columns 3-12. Cover plate and store at 4° C.

2. Use 10 PF1 plates filled in (1) to create the pools containing 10 samples.

(i) Using a programmable multichannel pipettor containing 8 pipette tips, aspirate 20 μL from each column of the PF1 plate until 200 μL total is collected in each pipette tip (i.e., 20 μL have been collected from each sample in a row of the PF1 plate).

(ii) Dispense the collected 200 μL into the first open column of the Plate Format 2 (PF2) plate, as shown in FIG. 38.

(iii) Repeat (i) and (ii) until columns 3-12 of the PF2 plate are filled.

(iv) Cover plate and store at 4° C.

3. Use 8 PF2 plates filled in (2) to create the pools containing 80 samples.

(i) Using a programmable multichannel pipettor containing 10 pipette tips, aspirate 20 μL from each row of the PF2 plate until 160 μL is collected in each pipette tip (i.e., 20 μL have been collected from each sample in a column of the PF2 plate).

(ii) Dispense the collected 160 μL into the first open row of the Plate Format 3 (PF3) plate, as shown in FIG. 38.

(iii) Repeat the pipetting until columns 3-12 of the PF3 plate are filled.

(iv) Cover plate and store at 4° C.

The PF3 plates contain pooled samples comprising 80 individual samples. The PF3 plates can be used with the methods described herein, e.g., to detect virus, viral component, and/or biomarker.

### Example 22. Uniformity Testing of Immobilized Antigens for Serology Assay

The uniformity test using anti-His antibody described in Examples 16 and 17 was also performed for two batches containing a total of 12 plates coated with SARS-CoV-2 S protein, N protein, and S-RBD. The plates were 384-well plates, each well containing four distinct binding domains (“spots”) as described herein, e.g., in FIG. 39A. The overall results are shown in Table 15 and FIGS. 40-42. As indicated in Table 15, the plates passed the specification threshold of 18% intra-plate coefficient of variation (CV).

TABLE 15
Inter-plate Signal Specifications

| | | CoV-2 Spike | CoV-2 N | CoV-2 RBD |
|---|---|---|---|---|
| CV of | Value | 13.4% | 6.6% | 10.4% |
| intraplate | Specification | 18.0% | 18.0% | 18.0% |
| averages | Result | Pass | Pass | Pass |
| | Overall | Pass | | |

FIGS. 40A and 40B show the average intra-plate CV, maximum intra-plate CV, average uniformity metric, maximum uniformity metric, and mean ECL signal for the two batches of plates. FIG. 41 shows the mean ECL signals from the four spots for the two batches of plates, and FIG. 42 shows the ECL signals from each of the three antigens on the plates as a percentage of the inter-plate mean signal. These results further demonstrate that the antigens were uniformly immobilized on the plates.

### Example 23. SARS-CoV-2 Single Nucleotide Polymorphism (SNP) Assay

Singleplex and multiplex oligonucleotide ligation assays (OLA) were performed to detect single nucleotide polymorphisms (SNPs) at SARS-CoV-2 genome locations 8782 (C>T mutation), 11083 (G>T mutation), 23403 (A>G mutation), and 28144 (T>C mutation). A pair of targeting probes and detection probes for each polymorphic site were designed to allow for single-base discrimination at the SNP site, as described in Example 9. Taq DNA ligase was used to join the targeting probe and detection probe that aligned correctly on the sample. Fragments of unmodified template complements were added to prevent bridging of unligated probes. The OLA cycling conditions were: 2 minutes at 95° C., then 30 cycles of 30 seconds at 95° C. and 2 minutes at 65° C. The plates were blocked with a blocking solution for 30 minutes at 37° C. during the OLA cycling.

The plate was washed, and the ligated probes were hybridized to a binding reagent on a plate. In the singleplex format, each well only contained the binding reagents specific for one SNP. In the multiplex format, each well of contained ten binding domains (“spots”), wherein a unique binding reagents was immobilized in each spot, allowing for detection of up to five SNPs per well. The hybridization was performed in hybridization buffer (50 μL per well), with a one-hour incubation at 37° C.

The hybridized ligated probes were then detected by streptavidin-SULFO-TAG™ as described in Example 9. Briefly, following the hybridization, the plate was washed, and a detection solution was added (50 μL per well) and incubated for 30 minutes at 37° C. The plate was washed, and read buffer was added (150 μL), and the plate was then read using a plate reader.

Ten (10) nasal swabs from COVID-19-positive patients obtained from BOCA Biolistics (Deerfield Beach, Fla.) were tested using both the singleplex and multiplex OLA assay formats. RNA was extracted using the MAGMAX™ Viral/Pathogen Ultra Nucleic Acid Isolation Kit (Applied Biosystems). RT-PCR was conducted using site-specific primers using TAQPATH™ 1-step RT-qPCR Master Mix (Applied Biosystems). Reference strain RNAs were obtained from BEI Resources. NIAID, NIH: Genomic RNA from SARS-Related Coronavirus 2, Isolate USA-WA1/2020, NR-52285. GenBankMN985325, deposited by the Centers for Disease Control and Prevention; Genomic RNA from SARS-Related Coronavirus 2, Isolate Hong Kong/VM20001061/2020, NR-52388, GenBankMT547814, deposited by the University of Hong Kong.

Results from a test assay using synthetic oligonucleotide template containing the SNPs of interest are shown FIG. 43. The synthetic oligonucleotide templates showed high specificity for the appropriate allele using ligation temperature of 65° C.

Results from the singleplex assay format with patient samples are shown in FIG. 44. There was a high prevalence of the L strain type (8782C and 28144T) that also have the D614G mutation. Only one strain had the “asymptomatic” allele (11083T). Results were also compared to a fully sequenced reference strain, with all results matching the published sequences for these strains. Results from the multiplex assay format with patient samples are shown in FIG. 45 and were consistent with the results from the singleplex assays shown in FIG. 44.

Reproducibility of the multiplex assay was also assessed. The multiplex RT-PCR products from the samples shown in FIG. 45 were subjected to three separate multiplexed OLA reactions on three different days to determine the allele frequency reproducibility from the SNP assays. The mean, standard deviation (STD), coefficient of variation (CV), and total number (N) of readings are shown in Table 16 for the samples that either had the WT or mutant (MUT) allele for the given SNP. As shown in Table 16, all assays had good allele frequency reproducibility, and the SNP determinations for all ten samples and the reference strain were consistent between the runs for all three experiments.

TABLE 16
OLA Reproducibility

| | WT samples (WT allele) | | | | MUT samples (MUT allele) | | | |
|---|---|---|---|---|---|---|---|---|
| | | | | Total | | | | Total |
| Site | Mean | STD | CV | N | Mean | STD | CV | N |
| 8782 | 97.8 | 3.1 | 0.03 | 54 | 98.6 | 0.3 | 0.003 | 6 |
| 11083 | 99.3 | 2.8 | 0.03 | 54 | 98.1 | 1.0 | 0.01 | 6 |
| 23403 | 99.2 | 0.4 | 0.004 | 12 | 100.3 | 5.4 | 0.05 | 48 |
| 28144 | 96.3 | 10.0 | 0.1 | 54 | 98.7 | 0.8 | 0.01 | 6 |

### Example 24. Detection of SARS-CoV-2 Nucleocapsid Protein in Human Samples

The Format 2 assay described in Example 7 was used to test for SARS-CoV-2 N protein in the following samples: nasopharyngeal swabs from 12 patients who tested positive for COVID-19, nasopharyngeal swabs from 6 patients who tested negative for COVID-19, and normal (COVID-19 negative) human saliva, serum, and EDTA plasma. The samples were all tested without dilution. Assay results of the SARS-CoV-2 N protein concentration are shown in FIG. 46. The results for the negative nasopharyngeal swab and normal saliva, serum, and EDTA plasma were comparable, while the positive nasopharyngeal swab samples had significantly higher concentration of SARS-CoV-2 N protein.

The dilution linearity and spike recovery of the assay were also tested to determine whether the assay is affected by component(s) that may be present in the biological sample matrix (also known as the “sample matrix effect”). For dilution linearity, the normal human serum, EDTA plasma, saliva, and COVID-19 negative human nasopharyngeal swab samples were spiked with calibrator and tested at different dilutions. Percent recovery at each dilution level was normalized to the dilution-adjusted, neat concentration, and shown in FIG. 47.

For spike recovery, normal human serum. EDTA plasma, saliva, and COVID-19 negative human nasopharyngeal swab samples were spiked with calibrator at three levels. Spiked samples were tested neat. Percent recovery is shown in FIG. 48. Based on the results in FIGS. 47 and 48, samples may be diluted to reduce sample matrix effects.

### Example 25. SARS-CoV-2 Nucleic Acid Detection Assay—OLA

The oligonucleotide ligation assay (OLA) described in Examples 9, 13, and 23 was also used to detect SARS-CoV-2 nucleic acid. Targeting and detection probes were designed for the SARS-CoV-2 N1, N2, and N3 regions (described in Lu et al., Emerg Infect Dis 26(8):1654-1665 (2020)) and the human RPP30 gene as control. The targeting probes have unique 5′ oligonucleotide tag sequences that are complementary to binding reagents on specific binding domains on a multi-well plate. The detection probes have a 5′ phosphate group for ligation and a 3′ biotin. OLA was used to ligate targeting and detection probes that aligned perfectly on the SARS-CoV-2 target nucleic acid. The ligated probes were then hybridized to the multi-well plate and detected by adding streptavidin-labeled SULFO-TAG™ and reading the signal with a plate reader. The plates were 10-spot, 96-well plates with the spot layout as shown in FIG. 39B. The binding reagents were immobilized in the spots as follows: Spot 4: binding reagent for N1; Spot 5: binding reagent for N2, Spot 9: binding reagent for N3; Spot 10: binding reagent for RPP30. The remaining spots were blank.

Probes were designed against both the viral RNA strand and the cDNA strand that is synthesized upon reverse transcription. The assay was first performed with a synthetic DNA template that included the target region (SARS-CoV-2 N1, N2, N3, and human RPP30). For viral RNA, one-step RT-PCR was performed to reverse transcribe and amplify the region surrounding each target. The OLA conditions are provided in Table 17.

TABLE 17
OLA Cycling Conditions

| | | |
|---|---|---|
| 95° C. | 2 min | |
| 95° C. | 30 sec | × 30 cycles |
| 65° C. | 2 min | |
| 4° C. | Hold | |

Two sets of targeting and detection probes for each site were tested, designated as “OLA1” or “OLA2.” The probes sequences are shown in Tables 18 and 19.

TABLE 18
Probes

| Probe Name | Probe sequence |
|---|---|
| N1 Targeting probe OLA1 | GTGGCAACAGAAATCAGGTGGTGA cgaaatgcac cccgcattac |
| | (SEQ ID NO: 22) |
| N1 Detection probe OLA1 | /5phos/gtttggtgga ccctcagatt c/3bio/ |
| | (SEQ ID NO: 23) |
| N1 Targeting probe OLA2 | GTGGCAACAGAAATCAGGTGGTGA gaatctgagggtccaccaaac |
| | (SEQ ID NO: 24) |
| N1 Targeting probe OLA2 | TAGATGCCGCTGCAGGTATGGAAA gaatctgagggtccaccaaac |
| | (SEQ ID NO: 25) |
| N1 Detection probe OLA2 | /5phos/gtaatgcggggtgcatttcg/3bio/ |
| | (SEQ ID NO: 26) |
| N2 Targeting probe OLA1 | TAGATGCCGCTGCAGGTATGGAAA gc aaattgcaca atttgcccc |
| | (SEQ ID NO: 27) |
| N2 Detection probe OLA1 | /5Phos/c agcgcttcag cgttctt/3bio/ |
| | (SEQ ID NO: 28) |
| N2 Targeting probe OLA2 | TAGATGCCGCTGCAGGTATGGAAA gaacgctgaagcgctgg |
| | (SEQ ID NO: 29) |
| N2 Detection probe OLA2 | /5phos/gggcaaattgtgcaatttgcg/3bio/ |
| | (SEQ ID NO: 30) |
| N3 Targeting probe OLA1 | AGAGGACTGCTAAAGGTTTGTAGG ccaa aagay cacat tggcacc |
| | (SEQ ID NO: 31) |
| N3 Detection probe OLA1 | /5phos/cgc aatcctgcta acaatgctg/3bio/ |
| | (SEQ ID NO: 32) |
| N3 Targeting probe OLA2 | AGAGGACTGCTAAAGGTTTGTAGG gcattgttagcaggattgcgg |
| | (SEQ ID NO: 33) |
| N3 Detection probe OLA2 | /5phos/gtgccaatgtgrtcttttggtg/3bio/ |
| | (SEQ ID NO: 34) |
| RPP30 Targeting probe OLA1 | CGTACCATTGAATCTGGAGACCTT gagcgggtt ctgacctga |
| | (SEQ ID NO: 35) |
| RPP30 Detection probe OLA1 | /5phos/aggctctgcgc ggact/3bio/ |
| | (SEQ ID NO: 36) |
| RPP30 Targeting probe OLA2 | CGTACCATTGAATCTGGAGACCTT caagtccgcgcagagcc |
| | (SEQ ID NO: 37) |
| RPP30 Detection probe OLA2 | /5phos/ttcaggtcagaacccgct/3bio/ |
| | (SEQ ID NO: 38) |

TABLE 19
Blocking Oligonucleotides

| Blocking | Blocking |
|---|---|
| Oligonucleotide Name | Oligonucleotide Sequence |
| N1 Targeting OLA1 BO | cgaaatgcac cccgcattac |
| | (SEQ ID NO: 39) |
| N1 Detection OLA1 BO | gtttggtgga ccctcagatt c |
| | (SEQ ID NO: 40) |
| N1 Targeting OLA2 BO | gaatctgagggtccaccaaac |
| | (SEQ ID NO: 41) |
| N1 Detection OLA2 BO | gtaatgcggggtgcatttcg |
| | (SEQ ID NO: 42) |
| N2 Targeting OLA1 BO | gc aaattgcaca atttgcccc |
| | (SEQ ID NO: 43) |
| N2 Detection OLA1 BO | c agcgcttcag cgttctt |
| | (SEQ ID NO: 44) |
| N2 Targeting OLA2 BO | gaacgctgaagcgctgg |
| | (SEQ ID NO: 45) |
| N2 Detection OLA2 BO | gggcaaattgtgcaatttgcg |
| | (SEQ ID NO: 46) |
| N3 Targeting OLA1 BO | ccaa aagay cacat tggcaacc |
| | (SEQ ID NO: 47) |
| N3 Detection OLA1 BO | cgc aatcctgcta acaatgctg |
| | (SEQ ID NO: 48) |
| N3 Targeting OLA2 BO | gcattgttagcaggattgcgg |
| | (SEQ ID NO: 49) |
| N3 Detection OLA2 BO | gtgccaatgtgrtcttttggtg |
| | (SEQ ID NO: 50) |
| RPP30 Targeting OLA1 BO | gagcgggtt ctgacctga |
| | (SEQ ID NO: 51) |
| RPP30 Detection OLA1 BO | aggctctgcgc ggact |
| | (SEQ ID NO: 52) |
| RPP30 Targeting OLA2 BO | caagtccgcgcagagcc |
| | (SEQ ID NO: 53) |
| RPP30 Detection OLA2 BO | ttcaggtcagaacccgct |
| | (SEQ ID NO: 54) |

Primers for amplification of the SARS-CoV-2 N1, N2, N3 regions and human RPP30 are described in Lu et al., Emerg Infect Dis 26(8):1654-1665 (2020) and reproduced in Table 20.

TABLE 20
Amplification Primers

| Primer Name | Primer Sequence |
|---|---|
| N1 Forward | GACCCCAAAATCAGCGAAAT |
| | (SEQ ID NO: 67) |
| N1 Reverse | TCTGGTTACTGCCAGTTGAATCTG |
| | (SEQ ID NO: 68) |
| N2 Forward | TTACAAACATTGGCCGCAAA |
| | (SEQ ID NO: 69) |
| N2 Reverse | GCGCGACATTCCGAAGAA |
| | (SEQ ID NO: 70) |
| N3 Forward | GGGAGCCTTGAATACACCAAAA |
| | (SEQ ID NO: 71) |
| N3 Reverse | TGTAGCACGATTGCAGCATTG |
| | (SEQ ID NO: 72) |
| RPP30 Forward | AGATTTGGACCTGCGAGCG |
| | (SEQ ID NO: 73) |
| RPP30 Reverse | GAGCGGCTGTCTCCACAAGT |
| | (SEQ ID NO: 74) |

The OLA assays were performed on RNA extracted from swab samples of SARS-CoV-2 positive (n=15) or negative (n=6) patients. Detectable signals for all of the same targets were observed as from the results of a qRT-PCR assay. The negative control samples did not have appreciable signal, indicating good specificity to SARS-CoV-2. N1 and N3 showed the best results. RPP30 signals were high in all samples (SARS-CoV-2 positive and negative), which was expected and confirmed consistency in RNA extraction.

### Example 26. SARS-CoV-2 Nucleic Acid Detection Assay—Internal Detection Probe

A SARS-CoV-2 nucleic acid detection assay that amplifies regions of interest (N1, N2, and N3) and contacts the amplified regions with a single internal detection probe was developed. The forward primer was tagged with a 5′ oligonucleotide tag. The internal detection probes have a Y biotin. The SARS-CoV-2 N1, N2, and N3 regions and the human RPP30 gene (control) were amplified using multiplexed PCR, followed by hybridization of the PCR products with the internal detection probes to form hybridized. The hybridized products were then immobilized to the multi-well plate and detected by adding streptavidin-labeled SULFO-TAG™ and reading the signal with a plate reader. The plates were 10-spot, 96-well plates with the spot layout as shown in FIG. 39B. The binding reagents were immobilized in the spots as follows: Spot 8: binding reagent for N1; Spot 9: binding reagent for N2, Spot 10: binding reagent for N3; Spot 1: binding reagent for RPP30. The remaining spots were blank.

Sequences of the forward primers, reverse primers, and the internal detection probes are shown in Table 21.

TABLE 21
Primers and Internal Detection Probe Sequences

| Assay | Forward primer | Reverse Primer | Internal Probe |
|---|---|---|---|
| N1 | AATCCGTCGACTAGCCTGAGAATT | TCTGGTTACTGCCAGTTG | ACC CCG CAT TAC GTT TGG |
| | GACCCCAAAATCAGCGAAAT | AATCTG | TGG ACC/3Bio/ |
| | (SEQ ID NO: 55) | (SEQ ID NO: 56) | (SEQ ID NO: 57) |
| N2 | AGAGGACTGCTAAAGGTTTGTAGG | GCGCGACATTCCGAAGAA | ACA ATT TGC CCC CAG CGC |
| | TTACAAACATTGGCCGCAAA | (SEQ ID NO: 59) | TTC AG/3Bio/ |
| | (SEQ ID NO: 58) | | (SEQ ID NO: 60) |
| N3 | CGTACCATTGAATCTGGAGACCTT | TGTAGCACGATTGCAGCA | AYCACA TTG GCA CCC GCA |
| | GGGAGCCTTGAATACACCAAAA | TTG | ATC CTG/3Bio/ |
| | (SEQ ID NO: 61) | (SEQ ID NO: 62) | (SEQ ID NO: 63) |
| RPP30 | ACTGGTAACCCAGACATGATCGGT | GAGCGGCTGTCTCCACAA | TTC TGA CCT GAA GGC TCT |
| | AGATTTGGACCTGCGAGCG | GT | GCG CG/3Bio/ |
| | (SEQ ID NO: 64) | (SEQ ID NO: 65) | (SEQ ID NO: 66) |

### Example 27. Detection of SARS-CoV-2 in Wastewater Samples

Five wastewater samples (250 mL each) were obtained from a commercially available source. The samples were shipped and stored at 2 to 8° C.

### Example 27A. Detection of SARS-CoV-2 N Protein

The Format 2 assay described in Example 7 was used to test for SARS-CoV-2 N protein in the five wastewater samples, with PBS-T as a negative control (blank). The samples were assayed either without treatment (“non-processed”), spiked with heat-inactivated SARS-CoV-2, or concentrated using a 10K AMICON® spin filter. The concentration factor was approximately 40 to 55-fold, i.e., a 15 mL sample was concentrated to approximately 150 to 400 μL. The samples were also spiked with 1% TRITON® X-100 prior to the concentration step.

Results of the assay are shown in FIGS. 49A to 49C. As shown in FIG. 49A, one sample (Sample #3) showed a significantly higher signal after the concentration step. FIG. 49B shows that the spike recovery of the samples varied from 9% to 94%, indicating that the presence of human antibodies may interfere with analyte detection. FIG. 49C shows near-perfect recovery of the N-protein in the PBS-T control after the concentration step. However, no significant increase in signal was observed from the concentrated wastewater samples, possibly reflecting the presence of anti-N antibodies. Thus, serology assays were performed to characterize the amounts, specificities, and isotypes of anti-viral antibodies as follows.

### Example 27B. Serology Assay

A preliminary immunoassay was performed to determine the amounts of IgG and IgA in stool as compared to serum. Results are shown in FIG. 50, with the underlined numbers indicating the beginning of a linear drop in signal upon dilution. The results showed that a measurable amount of IgG is present in stool samples, and the concentration of IgA is significantly higher.

Immunoassays were performed on the non-processed wastewater samples after 1 day or 27 days of storage at 4° C. to determine the amount of total IgA, IgG, and IgM. Results are shown in FIGS. 51A to 51C. FIG. 51A shows a significant variation in the antibody signals between the samples, and that IgA, IgG, and IgM levels all decreased with longer storage time, likely due to degradation at 4° C. FIG. 51B shows that IgA signal decreased with dilution. FIG. 51C shows that IgA signal increased with concentration.

Serology assay panel #15 in Table 6, as described in Example 3, was used to test for antibodies in four of the wastewater samples (Samples #2 to #5). The wastewater samples were either non-processed or concentrated and stored for at least 30 days at 2 to 8° C. Results for the measured IgA concentrations are shown in FIG. 52A. As shown in FIG. 52A, three of the four tested samples (Sample #2, 3, and 5) showed a high IgA signal for antibodies against SARS-CoV-2 N protein. FIG. 52C shows correlation between signals for IgA and IgG antibodies against the SARS-CoV-2 antigens (S-NTD, N protein, S protein, and S-RBD) in six concentrated wastewater samples. The same serology assay panel was used to test for antibodies present in a stool sample that was collected from a healthy subject prior to the emergence of SARS-CoV-2. The stool sample was tested at 10× or 100× dilution. The results are in FIG. 52B and show a high signal for antibodies against antigens from circulating coronaviruses (HCoV-OC43, HCoV-229E, HCoV-HKU1, HCoV-NL63).

### Example 28. SARS-CoV-2 Strain Typing and SNP Detection

The oligonucleotide ligation assay (OLA) as described in Examples 9, 13, and 23 is performed to detect mutations of the SARS-CoV-2 S protein. SARS-CoV-2 strains and the associated SNPs are shown in Table 1A and include genome locations 21765-21770, 23063, 23604, 22132, 22206, 22917, 23012, 23664, 22813, 22812, 22227, 28932, 29645, 1059, 25563, 21991-21993, 23271, 23709, 24506, 24914, 241, 3037, 14408, 26144, 29095, 22865, and 22320. Two sets of targeting and detection probes (“OLA1” or “OLA2”) for each genome location were designed. Further, two sets of targeting (“US”) probes for each genome location, one for the reference strain (“WT”) and one for the variant, were designed. The same detection (“DS”) probe for each genome location was designed for both the reference strain and the variant. The sequences for the targeting and detection probes are shown in Table 22. The sequences of synthetic templates containing the SARS-CoV-2 genome regions of interest are shown in Table 23. The sequences for blocking oligonucleotides are shown in Table 24. The primers for amplifying the target regions are shown in Table 25.

The OLA as described above is also performed to detect single polynucleotide polymorphisms (SNPs) at SARS-CoV-2 genome locations 8782, 28144, 23403, and 11083. As described herein, SNPs at genome locations 8782 and 28144 differentiate the L and S strains of the SARS-CoV-2. The A>G SNP at genome location 23403 encodes the D614G mutation in the SARS-CoV-2 S protein. The G>T SNP at genome location 11083 is associated with asymptomatic infection by SARS-CoV-2. The sequences for the targeting and detection probes are shown in Table 26. The sequences of synthetic templates containing the SARS-CoV-2 genome regions of interest are shown in Table 27. The sequences for blocking oligonucleotides are shown in Table 28.

TABLE 22
Targeting and Detection Probes-S Protein Mutations

| | | Protein/ |
|---|---|---|
| Probe Name | Probe sequence | Amino Acid |
| 21765-21770 WT US Probe OLA1 | ACTCCCTGTGGGTGAGCTTAATGG gttac ttggttcat | S protein |
| | gctatacatg (SEQ ID NO: 75) | Δ69-70 |
| 21765-21770 Del US Probe OLA1 | ACTGGTAACCCAGACATGATCGGT ccaatgttac ttggttccat | |
| | gcta (SEQ ID NO: 76) | |
| 21765-21770 DS Probe OLA1 | /5Phos/tctctgggaccaatggtact aag/3bio/ | |
| | (SEQ ID NO: 77) | |
| 21765-21770 WT US Probe OLA2 | ACTCCCTGTGGGTGAGCTTAATGG ccattg gtcccagagacatgta | |
| | (SEQ ID NO: 78) | |
| 21765-21770 Del US Probe OLA2 | ACTGGTAACCCAGACATGATCGGT cttagtaccattg gtcccagaga | |
| | (SEQ ID NO: 79) | |
| 21765-21770 DS Probe OLA2 | /5Phos/tagcatggaaccaagtaacattgg/3Bio/ | |
| | (SEQ ID NO: 80) | |
| 23063A US Probe OLA1 | CATTTGGTCATTGGTTCAAGACGA aatcatatggt ttccaaccca | S protein |
| | cta (SEQ ID NO: 81) | N501Y |
| 23063T US Probe OLA1 | CTGGTCCGTTGTGGTCCTTCTAAC a atcatatggt ttccaaccca | |
| | ctt (SEQ ID NO: 82) | |
| 23063 DS Probe OLA1 | /5Phos/atggtgt tggttaccaa ccatac/3Bio/ | |
| | (SEQ ID NO: 83) | |
| 23063A US Probe OLA2 | CATTTGGTCATTGGTTCAAGACGA tatggttggtaaccaacaccatt | |
| | (SEQ ID NO: 84) | |
| 23063T US Probe OLA2 | CTGGTCCGTTGTGGTCCTTCTAAC tatggttggtaaccaacaccata | |
| | (SEQ ID NO: 85) | |
| 23063 DS Probe OLA2 | /5Phos/agtgggttggaaaccatatgat tg/3Bio/ | |
| | (SEQ ID NO: 86) | |
| 23604C US Probe OLA1 | GTGGCAACAGAAATCAGGTGGTGA ctag ttatcagact | S protein |
| | cagactaatt ctcc (SEQ ID NO: 87) | P681H |
| 23604A US Probe OLA1 | AGAGGACTGCTAAAGGTTTGTAGG ctag ttatcagact | |
| | cagactaatt ctca (SEQ ID NO: 88) | |
| 23604 DS Probe OLA1 | /5Phos/tcggcg ggcacgtag/3Bio/ (SEQ ID NO: 89) | |
| 23604C US Probe OLA2 | GTGGCAACAGAAATCAGGTGGTGA ctacgtgcccgccgag | |
| | (SEQ ID NO: 90) | |
| 23604A US Probe OLA2 | AGAGGACTGCTAAAGGTTTGTAGG ctacgtgcccgccgat | |
| | (SEQ ID NO: 91) | |
| 23604 DS Probe OLA2 | /5Phos/gagaattagtctgagtctgataa ctagc/3Bio/ | |
| | (SEQ ID NO: 92) | |
| 22132G US Probe OLA1 | CTGGTCCGTTGTGGTCCTTCTAAC ggaaaaca gggtaatttc | S protein |
| | aaaaatctta gg (SEQ ID NO: 93) | R190S |
| 22132T US Probe OLA1 | CATTTGGTCATTGGTTCAAGACGA ggaaaaca gggtaatttc | |
| | aaaaatctta gt (SEQ ID NO: 94) | |
| 22132 DS Probe OLA1 | /5Phos/gaattgt gtttaagaat attgatggtt attttaaaat | |
| | at/3Bio/ (SEQ ID NO: 95) | |
| 22132G US Probe OLA2 | CTGGTCCGTTGTGGTCCTTCTAAC taaaataaccatcaatattcttaa | |
| | acacaaattcc (SEQ ID NO: 96) | |
| 22132T US Probe OLA2 | CATTTGGTCATTGGTTCAAGACGA taaaataaccatcaatattcttaa | |
| | acacaaattca (SEQ ID NO: 97) | |
| 22132 DS Probe OLA2 | /5Phos/ctaagatttttgaaattaccctgtttcct/3Bio/ | |
| | (SEQ ID NO: 98) | |
| 22206A US Probe OLA1 | AATCCGTCGACTAGCCTGAGAATT acgccta ttaatttagt | S protein |
| | gcgtga (SEQ ID NO: 99) | D215G |
| 22206G US Probe OLA1 | CTAATAGCTCCTGTGCCCTCGTAT acgccta ttaatttagt | |
| | gcgtgg (SEQ ID NO: 100) | |
| 22206 US Probe OLA1 | /5Phos/tctc cctcagggtt tttcgg/3Bio/ | |
| | (SEQ ID NO: 101) | |
| 22206A US Probe OLA2 | AATCCGTCGACTAGCCTGAGAATT ccgaaaaaccctgagggagat | |
| | (SEQ ID NO: 102) | |
| 22206G US Probe OLA2 | CTAATAGCTCCTGTGCCCTCGTAT ccgaaaaaccctgagggagac | |
| | (SEQ ID NO: 103) | |
| 22206 DS Probe OLA2 | /5Phos/cacgc actaaattaataggcgtg/3Bio/ | |
| | (SEQ ID NO: 104) | |
| 22917T US Probe OLA1 | AGAGGACTGCTAAAGGTTTGTAGG attc taaggttggt | S protein |
| | ggtaattata attacct (SEQ ID NO: 105) | L452R |
| 22917G US Probe OLA1 | GTGGCAACAGAAATCAGGTGGTGA attc taaggttggt | |
| | ggtaattata attaccg (SEQ ID NO: 106) | |
| 22917 DS Probe OLA1 | /5Phos/gta tagattgttt aggaagtcta atctcaaac/3Bio/ | |
| | (SEQ ID NO: 107) | |
| 22917T US Probe OLA2 | AGAGGACTGCTAAAGGTTTGTAGG gtttgagattagacttcctaaaca | |
| | atcta taca (SEQ ID NO: 108) | |
| 22917G US Probe OLA2 | GTGGCAACAGAAATCAGGTGGTGA gtttgagattagacttcctaaaca | |
| | atcta tacc (SEQ ID NO: 109) | |
| 22917 DS Probe OLA2 | /5Phos/ggtaattataattaccaccaaccttagaat/3Bio/ | |
| | (SEQ ID NO: 110) | |
| 23012G US Probe OLA1 | ACTGGTAACCCAGACATGATCGGT agcacacctt gtaatggtgt tg | S protein |
| | (SEQ ID NO: 111) | E484K |
| 23012A US Probe OLA1 | ACTCCCTGTGGGTGAGCTTAATGG agcacacctt gtaatggtgt ta | |
| | (SEQ ID NO: 112) | |
| 23012 DS Probe OLA1 | /5Phos/aaggtttt aattgttact ttcctttaca atc/3Bio/ | |
| | (SEQ ID NO: 113) | |
| 23012G US Probe OLA2 | ACTGGTAACCCAGACATGATCGGT tgat tgtaaaggaaagtaacaat | |
| | taaaaccttc (SEQ ID NO: 114) | |
| 23012A US Probe OLA2 | ACTCCCTGTGGGTGAGCTTAATGG tgat tgtaaaggaaagtaacaat | |
| | taaaaccttt (SEQ ID NO: 115) | |
| 23012 DS Probe OLA2 | /5Phos/aacaccattacaaggtgtgcta/3Bio/ | |
| | (SEQ ID NO: 116) | |
| 23664C US Probe OLA1 | TAGATGCCGCTGCAGGTATGGAAA gcctacact atgtcacttg | S protein |
| | gtgc (SEQ ID NO: 117) | A701V |
| 23664T US Probe OLA1 | CGTACCATTGAATCTGGAGACCTT gcctacact atgtcacttg | |
| | gtgt (SEQ ID NO: 118) | |
| 23664 DS Probe OLA1 | /5Phos/agaaaa ttcagttgct tactctaata actct/3Bio/ | |
| | (SEQ ID NO: 119) | |
| 23664C US Probe OLA2 | TAGATGCCGCTGCAGGTATGGAAA agagttattagagtaagcaactga | |
| | attttctg (SEQ ID NO: 120) | |
| 23664T US Probe OLA2 | CGTACCATTGAATCTGGAGACCTT agagttattagagtaagcaactga | |
| | attttcta (SEQ ID NO: 121) | |
| 23664 DS Probe OLA2 | /5Phos/caccaagtgacatagtgtaggc/3Bio/ | |
| | (SEQ ID NO: 122) | |
| 22813G US Probe OLA1 | TAGATGCCGCTGCAGGTATGGAAA gctccagg gcaaactgga aMg | S protein |
| | (SEQ ID NO: 123) | K417N |
| 22813T US Probe OLA1 | CGTACCATTGAATCTGGAGACCTT gctccagg gcaaactgga aMt | |
| | (SEQ ID NO: 124) | |
| 22813 DS Probe OLA1 | /5Phos/attgctg attataatta taaattacca gatgatttta | |
| | c/3Bio/ (SEQ ID NO: 125) | |
| 22813G US Probe OLA2 | TAGATGCCGCTGCAGGTATGGAAA gtaaaatcatctggtaatttataa | |
| | ttataatcagcaatc (SEQ ID NO: 126) | |
| 22813T US Probe OLA2 | CGTACCATTGAATCTGGAGACCTT gtaaaatcatctggtaatttataa | |
| | ttataatcagcaata (SEQ ID NO: 127) | |
| 22813 DS Probe OLA2 | /5Phos/Kttccagtttgc cctggagc/3Bio/ | |
| | (SEQ ID NO: 128) | |
| 22812A US Probe OLA1 | CTGGTCCGTTGTGGTCCTTCTAAC gctccagg gcaaactgga aa | S protein |
| | (SEQ ID NO: 129) | K417T |
| 22812C US Probe OLA1 | CATTTGGTCATTGGTTCAAGACGA gctccagg gcaaactgga ac | |
| | (SEQ ID NO: 130) | |
| 22812 DS Probe OLA1 | /5Phos/Kattgctg attataatta taaattacca gatgatttta | |
| | c/3Bio/ (SEQ ID NO: 131) | |
| 22812A US Probe OLA2 | CTGGTCCGTTGTGGTCCTTCTAAC atcatctggtaatttataattata | |
| | atcagcaatMt (SEQ ID NO: 132) | |
| 22812C US Probe OLA2 | CATTTGGTCATTGGTTCAAGACGA atcatctggtaatttataattata | |
| | atcagcaatMg (SEQ ID NO: 133) | |
| 22812 DS Probe OLA2 | /5Phos/ttccagtttgc cctggagc/3Bio/ | |
| | (SEQ ID NO: 134) | |
| 22227C US Probe OLA1 | tc cctcagggtt tttcggc (SEQ ID NO: 227) | S protein |
| 22227T US Probe OLA1 | tc cctcagggtt tttcggt (SEQ ID NO: 228) | A222V |
| 22227 DS Probe OLA1 | /5Phos/ttt agaccattg gtagatttgc ca/3Bio/ | |
| | (SEQ ID NO: 229) | |
| 22227C US Probe OLA2 | ggcaaatctaccaatggttctaaag (SEQ ID NO: 230) | |
| 22227T US Probe OLA2 | ggcaaatctaccaatggttctaaaa (SEQ ID NO: 231) | |
| 22227 DS Probe OLA2 | /5Phos/ccgaaaaaccctgagggaga/3Bio/ | |
| | (SEQ ID NO: 232) | |
| 28932C US Probe OLA1 | cggtga tgctgctctt gc (SEQ ID NO: 233) | N protein |
| | | A220V |
| 28932T US Probe OLA1 | cggtga tgctgctctt gt (SEQ ID NO: 234) | |
| | /5Phos/tttgctgc tgcttgacag attg/3Bio/ | |
| 28932 DS Probe OLA1 | (SEQ ID NO: 235) | |
| 28932C US Probe OLA2 | caatctgtcaagcagcagcaaag (SEQ ID NO: 236) | |
| 28932T US Probe OLA2 | caatctgtcaagcagcagcaaaa (SEQ ID NO: 237) | |
| 28932 DS Probe OLA2 | /5Phos/caagagcagcatcaccgc/3Bio/ (SEQ ID NO: 238) | |
| 29645G US Probe OLA1 | tg aattctcgta actacatagc acaag (SEQ ID NO: 239) | Orf10 |
| 29645T US Probe OLA1 | tg aattctcgta actacatagc acaat (SEQ ID NO: 240) | V30L |
| 29645 DS Probe OLA1 | /5Phos/tagat gtagttaact ttaatctcac atagc/3Bio/ | |
| | (SEQ ID NO: 241) | |
| 29645G US Probe OLA2 | gctatgtgagattaaagttaactacatctac (SEQ ID NO: 242) | |
| 29645T US Probe OLA2 | gctatgtgagattaaagttaactacatctaa (SEQ ID NO: 243) | |
| 29645 DS Probe OLA2 | /5Phos/ttgtgctatgtagttacgagaattca/3Bio | |
| | (SEQ ID NO: 244) | |
| 1059C US Probe OLA1 | tt gaaattaaat tggcaaagaa atttgacac | Orf1a |
| | (SEQ ID NO: 245) | |
| 1059T US Probe OLA1 | tt gaaattaaat tggcaaagaa atttgacat | T265I |
| | (SEQ ID NO: 246) | |
| 1059 DS Probe OLA1 | /5Phos/c ttcaatgggg aatgtccaaa ttttg/3Bio/ | |
| | (SEQ ID NO: 247) | |
| 1059C US Probe OLA2 | aaaatttggacattccccattgaagg (SEQ ID NO: 248) | |
| 1059T US Probe OLA2 | aaaatttggacattccccattgaaga (SEQ ID NO: 249) | |
| 1059 DS Probe OLA2 | /5Phos/tgtcaaatttctttgccaatttaatttcaaa/3Bio/ | |
| | (SEQ ID NO: 250) | |
| 25563G US Probe OLA1 | g ttgcacttct tgctgttttt cag (SEQ ID NO: 251) | Orf3a |
| 25563T US Probe OLA1 | g ttgcacttct tgctgttttt cat (SEQ ID NO: 252) | Q57H |
| 25563 DS Probe OLA1 | /5Phos/agcgctt ccaaaatcat aaccct/3Bio/ | |
| | (SEQ ID NO: 253) | |
| 25563G US Probe OLA2 | gggttatgattttggaagcgctc (SEQ ID NO: 254) | |
| 25563T US Probe OLA2 | gggttatgattttggaagcgcta (SEQ ID NO: 255) | |
| 25563 DS Probe OLA2 | /5Phos/tgaaaaacagcaagaagtgcaac/3Bio/ | |
| | (SEQ ID NO: 256) | |
| 21991-21993 WT US Probe OLA1 | ttttgt aatgatccat ttttgggtgt tta (SEQ ID NO: 257) | S protein |
| 21991-21993 del US Probe OLA1 | aattttgt aatgatccat ttttgggtgt (SEQ ID NO: 258) | Y144 del |
| 21991-21993 DS US Probe OLA1 | /5Phos/ttaccac aaaaacaaca aaagttggat/3Bio/ | |
| | (SEQ ID NO: 259) | |
| 21991-21993 WT US Probe OLA2 | ccaacttttgttgtttttgtggtaataa (SEQ ID NO: 260) | |
| 21991-21993 del US Probe OLA2 | atccaacttttgttgtttttgtggtaa (SEQ ID NO: 261) | |
| 21991-21993 DS US Probe OLA2 | /5Phos/acacccaaaaatggatcattacaaaatt/3Bio/ | |
| | (SEQ ID NO: 262) | |
| 23271C US Probe OLA1 | cca acaatttggc agagacattg c (SEQ ID NO: 263) | S protein |
| 23271A US Probe OLA1 | cca acaatttggc agagacattg a (SEQ ID NO: 264) | A570D |
| 23271 DS Probe OLA1 | /5Phos/tgacactac tgatgctgtc cg/3Bio/ | |
| | (SEQ ID NO: 265) | |
| 23271C US Probe OLA2 | cggacagcatca gtagtgtcag (SEQ ID NO: 266) | |
| 23271A US Probe OLA2 | cggacagcatca gtagtgtcat (SEQ ID NO: 267) | |
| 23271 DS Probe OLA2 | /5Phos/caatgtctctgccaaattgttgga/3Bio/ | |
| | (SEQ ID NO: 268) | |
| 23709C US Probe OLA1 | tctaata actctattgc catacccac (SEQ ID NO: 269) | S protein |
| 23709T US Probe OLA1 | tctaata actctattgc catacccat (SEQ ID NO: 270) | T716I |
| 23709 DS Probe OLA1 | /5Phos/a aattttacta ttagtgttac cacagaaatt c/3Bio/ | |
| | (SEQ ID NO: 271) | |
| 23709C US Probe OLA2 | gaatttctgtggtaacactaatagtaaaattg (SEQ ID NO: 272) | |
| 23709T US Probe OLA2 | gaatttctgtggtaacactaatagtaaaattta (SEQ ID NO: 273) | |
| 23709 DS Probe OLA2 | /5Phos/tgggtatg gcaatagagttattagagtaa/3Bio/ | |
| | (SEQ ID NO: 274) | |
| 24506T US Probe OLA1 | gcaat ttcaagtgtt ttaaatgata tccttt | S protein |
| | (SEQ ID NO: 275) | |
| 24506G US Probe OLA1 | gcaat ttcaagtgtt ttaaatgata tccttg | S982A |
| | (SEQ ID NO: 276) | |
| 24506 DS Probe OLA1 | /5Phos/cacg tcttgacaaa gttgaggc/3Bio/ | |
| | (SEQ ID NO: 277) | |
| 24506T US Probe OLA2 | gcctcaactttgtcaagacgtga (SEQ ID NO: 278) | |
| 24506G US Probe OLA2 | gcctcaactttgtcaagacgtgc (SEQ ID NO: 279) | |
| 24506 DS Probe OLA2 | /5Phos/aaggatatcatttaaaacacttgaa attgca/3Bio/ | |
| | (SEQ ID NO: 280) | |
| 24914G US Probe OLA1 | gaattttt atgaaccaca aatcattact acag | S protein |
| | (SEQ ID NO: 281) | D1118H |
| 24914C US Probe OLA1 | gaattttt atgaaccaca aatcattact acac | |
| | (SEQ ID NO: 282) | |
| 24914 DS Probe OLA1 | /5Phos/acaaca catttgtgtc tggtaact/3Bio/ | |
| | (SEQ ID NO: 283) | |
| 24914G US Probe OLA2 | gttaccagacacaaatgtgttgtc (SEQ ID NO: 284) | |
| 24914C US Probe OLA2 | gttaccagacacaaatgtgttgtg (SEQ ID NO: 285) | |
| 24914 DS Probe OLA2 | /5Phos/tgtagtaatgatt tgtggttcataaaaattcc/3Bio/ | |
| | (SEQ ID NO: 286) | |
| 241C US Probe OLA1 | cga tcatcagcac atctaggtttc (SEQ ID NO: 287) | 5′ UTR |
| 241T US Probe OLA1 | cga tcatcagcac atctaggtttt (SEQ ID NO: 288) | |
| 241 DS Probe OLA1 | /5Phos/gtccgggtg tgaccgaaag/3Bio (SEQ ID NO: 289) | |
| 241C US Probe OLA2 | cggtcacacccggacg (SEQ ID NO: 290) | |
| 241T US Probe OLA2 | cggtcacacccggaca (SEQ ID NO: 291) | |
| 241 DS Probe OLA2 | /5Phos/aaacctagatgtgctgatgatcg/3Bio/ | |
| | (SEQ ID NO: 292) | |
| 3037C US Probe OLA1 | a ttggcttcac atatgtattg ttctttc (SEQ ID NO: 293) | nsp3 |
| 3037T US Probe OLA1 | a ttggcttcac atatgtattg ttctttt (SEQ ID NO: 294) | F924F |
| 3037 DS Probe OLA1 | /5Phos/tac cctccagatg aggatgaaga/3Bio/ | |
| | (SEQ ID NO: 295) | |
| 3037C US Probe OLA2 | cttcatcctcatctggagggtag (SEQ ID NO: 296) | |
| 3037T US Probe OLA2 | cttcatcctcatctggagggtaa (SEQ ID NO: 297) | |
| 3037 DS Probe OLA2 | /5Phos/aaagaacaatacatatgtgaagccaatt/3Bio/ | |
| | (SEQ ID NO: 298) | |
| 14408C US Probe OLA1 | ttttatt ctctacagtg ttcccacc (SEQ ID NO: 299) | RdRp |
| 14408T US Probe OLA1 | ttttatt ctctacagtg ttcccact (SEQ ID NO: 300) | P323L |
| 14408 DS Probe OLA1 | /5Phos/ta caagttttgg accactagtg aga/3Bio/ | |
| | (SEQ ID NO: 301) | |
| 14408C US Probe OLA2 | ctcactagtggtccaaaacttgtag (SEQ ID NO: 302) | |
| 14408T US Probe OLA2 | ctcactagtggtccaaaacttgtaa (SEQ ID NO: 303) | |
| 14408 DS Probe OLA2 | /5Phos/gtgggaacactgtagagaataaaact/3Bio/ | |
| | (SEQ ID NO: 304) | |
| 26144G US Probe OLA1 | gtccaaatt cacacaatcg acgg (SEQ ID NO: 305) | Orf3a |
| 26144T US Probe OLA1 | gtccaaatt cacacaatcg acgt (SEQ ID NO: 306) | G251V |
| 26144 DS Probe OLA1 | /5Phos/ttcatccgggagttgttaatccag/3Bio/ | |
| | (SEQ ID NO: 307) | |
| 26144G US Probe OLA2 | ctggattaacaactccggatgaac (SEQ ID NO: 308) | |
| 26144T US Probe OLA2 | ctggattaacaactccggatgaaa (SEQ ID NO: 309) | |
| 26144 DS Probe OLA2 | /5Phos/cgtcgattgtgtgaatttggaca/3Bio/ | |
| | (SEQ ID NO: 310) | |
| 29095C US Probe OLA1 | a agcatacacaat gtaacacaag ctttc (SEQ ID NO: 311) | N protein |
| 29095T US Probe OLA1 | a agcatacaat gtaacacaag ctttt (SEQ ID NO: 312) | F274F |
| 29095 DS Probe OLA1 | /5Phos/ggcag acgtggtcca gaa/3Bio/ (SEQ ID NO: 313) | |
| 29095C US Probe OLA2 | ctggaccacgtctgccg (SEQ ID NO: 314) | |
| 29095T US Probe OLA2 | ctggaccacgtctgcca (SEQ ID NO: 315) | |
| 29095 DS Probe OLA2 | /5Phos/aaagcttgtgttacattgtatgcttta/3Bio/ | |
| | (SEQ ID NO: 316) | |
| 22865G US Probe OLA1 | ca gatgatttta caggctgcgt tatag (SEQ ID NO: 317) | S protein |
| 22865T US Probe OLA1 | ca gatgatttta caggctgcgt tatat (SEQ ID NO: 318) | A435S |
| 22865 DS Probe OLA1 | /5Phos/cttgg aattctaaca atcttgattc taagg/3Bio/ | |
| | (SEQ ID NO: 319) | |
| 22865G US Probe OLA2 | cttagaatcaagattgttagaattccaagc (SEQ ID NO: 320) | |
| 22865T US Probe OLA2 | cttagaatcaagattgttagaattccaaga (SEQ ID NO: 321) | |
| 22865 DS Probe OLA2 | /5Phos/tataacgcagcctgtaaaatcatct/3Bio/ | |
| | (SEQ ID NO: 322) | |
| 22320A US Probe OLA1 | CGTACCATTGAATCTGGAGACCTT acataga agttatttga | S protein |
| | ctcctggtg a (SEQ ID NO: 533) | D253G |
| 22320G US Probe OLA1 | TAGATGCCGCTGCAGGTATGGAAA acataga agttatttga | |
| | ctcctggtg g (SEQ ID NO: 535) | |
| 22320 DS Probe OLA1 | /5Phos/ttcttcttca ggttggacag ctg/3Bio/ | |
| | (SEQ ID NO: 535) | |
| 22320A US Probe OLA2 | CGTACCATTGAATCTGGAGACCTT ag ctgtccaacc tgaagaagaa | |
| | t (SEQ ID NO: 536) | |
| 22320G US Probe OLA2 | TAGATGCCGCTGCAGGTATGGAAA ag ctgtccaacc tgaagaagaa | |
| | c (SEQ ID NO: 537) | |
| 22320 DS Probe OLA2 | /5Phos/caccaggag tcaaataact tctatgtaaa/3Bio/ | |
| | (SEQ ID NO: 538) | |

TABLE 23
Synthetic Templates S Protein Mutations

| | | Protein/ |
|---|---|---|
| | | Amino |
| Oligo Name | Sequence | Acid |
| 21765-21770 WT | cctttctttt ccaatgttac ttggttccat gcta | S protein |
| template | tacatg tctctgggac caatggtact aagaggtttg | Δ69-70 |
| strand | ataac (SEQ ID NO: 135) | |
| 21765-21770del | a cctttctttt ccaatgttac ttggttccat gcta | |
| template | tctctgggac caatggtact aagaggtttg | |
| strand | ataaccctgt (SEQ ID NO: 136) | |
| 21765-21770 WT | ttat caaacctctt agtaccattg gtcccagaga | |
| cDNA strand | catgta tagcatggaa ccaagtaaca ttggaaaaga | |
| | aaggt (SEQ ID NO: 137) | |
| 21765-21770del | agggttat caaacctctt agtaccattg gtcccagaga | |
| cDNA strand | tagcatggaa ccaagtaaca ttggaaaaga aaggtaa | |
| | (SEQ ID NO: 138) | |
| 23063A | ttact ttcctttaca atcatatggt ttccaaccca | S protein |
| template | ct a atggtgt tggttaccaa ccatacagag | N501Y |
| strand | tagtagtact (SEQ ID NO: 139) | |
| 23063T | ttact ttcctttaca atcatatggt ttccaaccca | |
| template | ct t atggtgt tggttaccaa ccatacagag | |
| strand | tagtagtact (SEQ ID NO: 140) | |
| 23063A cDNA | agtacta ctactctgta tggttggtaa ccaacaccat | |
| strand | t agtgggttgg aaaccatatg attgtaaagg | |
| | aaagtaa (SEQ ID NO: 141) | |
| 23063T cDNA | agtacta ctactctgta tggttggtaa ccaacaccat | |
| strand | a agtgggttgg aaaccatatg attgtaaagg | |
| | aaagtaa (SEQ ID NO: 142) | |
| 23604C | aggta tatgcgctag ttatcagact cagactaatt | S protein |
| template | ctc c tcggcg ggcacgtagt gtagctagtc | P681H |
| strand | aatccatcat (SEQ ID NO: 143) | |
| 23604A | aggta tatgcgctag ttatcagact cagactaatt | |
| template | ctc a tcggcg ggcacgtagt gtagctagtc | |
| strand | aatccatcat (SEQ ID NO: 144) | |
| 23604C cDNA | atgatggatt gactagctac actacgtgcc | |
| strand | cgccga g gagaattagt ctgagtctga | |
| | taactagcgc atatacct | |
| | (SEQ ID NO: 145) | |
| 23604A cDNA | atgatggatt gactagctac actacgtgcc cgccga | |
| strand | t gagaattagt ctgagtctga taactagcgc | |
| | atatacct (SEQ ID NO: 146) | |
| 22132G | accttg aaggaaaaca gggtaatttc aaaaatctta | S protein |
| template | g g gaatttgt gtttaagaat attgatggtt | R190S |
| strand | attttaaaatat (SEQ ID NO: 147) | |
| 22132T | accttg aaggaaaaca gggtaatttc aaaaatctta | |
| template | g t gaatttgt gtttaagaat attgatggtt | |
| strand | attttaaaatat (SEQ ID NO: 148) | |
| 22132G cDNA | ttttaaaat aaccatcaat attcttaaac acaaattc | |
| strand | c ctaagatttt tgaaattacc ctgttttcct | |
| | tcaaggt (SEQ ID NO: 149) | |
| 22132T cDNA | ttttaaaat aaccatcaat attcttaaac acaaattc | |
| strand | a ctaagatttt tgaaattacc ctgttttcct | |
| | tcaaggt (SEQ ID NO: 150) | |
| 22206A | at atattctaag cacacgccta ttaatttagt | S protein |
| template | gcgtg a tctc cctcagggtt tttcggcttt | D215G |
| strand | agaaccattg gta (SEQ ID NO: 151) | |
| 22206G | at atattctaag cacacgccta ttaatttagt | |
| template | gcgtg g tctc cctcagggtt tttcggcttt | |
| strand | agaaccattg gta (SEQ ID NO: 152) | |
| 22206A | taccaat ggttctaaag ccgaaaaacc | |
| cDNA | ctgagggaga t cacgc actaaattaa | |
| strand | taggcgtgtg cttagaatat at | |
| | (SEQ ID NO: 153) | |
| 22206G | taccaat ggttctaaag ccgaaaaacc | |
| cDNA | ctgagggaga c cacgc actaaattaa | |
| strand | taggcgtgtg cttagaatat at | |
| | (SEQ ID NO: 154) | |
| 22917T | a atcttgattc taaggttggt ggtaattata | S protein |
| template | attacc t gta tagattgttt aggaagtcta | L452R |
| strand | atctcaaacc tttt (SEQ ID NO: 155) | |
| 22917G | a atcttgattc taaggttggt ggtaattata | |
| template | attacc g gta tagattgttt aggaagtcta | |
| strand | atctcaaacc tttt (SEQ ID NO: 156) | |
| 22917T | aaaa ggtttgagat tagacttcct aaacaatcta | |
| cDNA | tac a ggtaattata attaccacca accttagaat | |
| strand | caagatt (SEQ ID NO: 157) | |
| 229170 | aaaa ggtttgagat tagacttcct aaacaatcta | |
| cDNA | tac c ggtaattata attaccacca accttagaat | |
| strand | caagatt (SEQ ID NO: 158) | |
| 23012G | aatcta tcaggccggt agcacacctt gtaatggtgt | S protein |
| template | t g aaggtttt aattgttact ttcctttaca | E484K |
| strand | atcatatggt (SEQ ID NO: 159) | |
| 23012A | aatcta tcaggccggt agcacacctt gtaatggtgt | |
| template | t a aaggtttt aattgttact ttcctttaca | |
| strand | atcatatggt (SEQ ID NO: 160) | |
| 23012G | ccatatg attgtaaagg aaagtaacaa ttaaaacctt | |
| cDNA | c aacaccatta caaggtgtgc taccggcctg | |
| strand | atagatt (SEQ ID NO: 161) | |
| 23012A | ccatatg attgtaaagg aaagtaacaa ttaaaacctt | |
| cDNA | t aacaccatta caaggtgtgc taccggcctg | |
| strand | atagatt (SEQ ID NO: 162) | |
| 23664C | agtc aatccatcat tgcctacact atgtcacttg | S protein |
| template | gtg c agaaaa ttcagttgct tactctaata | A701V |
| strand | actctattgc c (SEQ ID NO: 163) | |
| 23664T | agtc aatccatcat tgcctacact atgtcacttg | |
| template | gtg t agaaaa ttcagttgct tactctaata | |
| strand | actctattgc c (SEQ ID NO: 164) | |
| 23664C | ggcaata gagttattag agtaagcaac tgaattttct | |
| cDNA | g caccaagtga catagtgtag gcaatgatgg | |
| strand | attgact (SEQ ID NO: 165) | |
| 23664T | ggc-aata gagttattag agtaagcaac tgaattttct | |
| cDNA | a caccaagtga catagtgtag gcaatgatgg | |
| strand | attgact (SEQ ID NO: 166) | |
| 22813G | atgaa gtcagacaaa tcgctccagg gcaaactgga | S protein |
| template | aM g attgctg attataatta taaattacca | K417N |
| strand | gatgatttta (SEQ ID NO: 167) | |
| 22813T | atgaa gtcagacaaa tcgctccagg gcaaactgga | |
| template | aM t attgctg attataatta taaattacca | |
| strand | gatgatttta (SEQ ID NO: 168) | |
| 22813C | taaaatc atctggtaat ttataattat aatcagcaat | |
| cDNA | c Kttccagttt gccctggagc gatttgtctg | |
| strand | acttcat (SEQ ID NO: 169) | |
| 22813T | taaaatc atctggtaat ttataattat aatcagcaat | |
| cDNA | a Kttccagttt gccctggagc gatttgtctg | |
| strand | acttcat (SEQ ID NO: 170) | |
| 22812A | gatgaa gtcagacaaa tcgctccagg gcaaactgga | S protein |
| template | a a Kattgctg attataatta taaattacca | K417T |
| strand | gatgatttt (SEQ ID NO: 171) | |
| 22812C | gatgaa gtcagacaaa tcgctccagg gcaaactgga | |
| template | a c Kattgctg attataatta taaattacca | |
| strand | gatgatttt (SEQ ID NO: 172) | |
| 22812A | aaaatca tctggtaatt tataattata atcagcaatM | |
| cDNA | t ttccagtttg ccctggagcg atttgtctga | |
| strand | cttcatc (SEQ ID NO: 173) | |
| 22812C | aaaatca tctggtaatt tataattata atcagcaa | |
| cDNA | tM g ttccagtttg ccctggagcg atttgtctga | |
| strand | cttcatc (SEQ ID NO: 174) | |
| 22227C | a ttaatttagt gcgtgatctc cctcagggtt | S protein |
| template | tttcgg c ttt agaaccattg gtagatttgc | A222V |
| strand | caataggtat taac (SEQ ID NO: 323) | |
| 22227T | a ttaatttagt gcgtgatctc cctcagggtt | |
| template | tttcgg t ttt agaaccattg gtagatttgc | |
| strand | caataggtat taac (SEQ ID NO: 324) | |
| 22227C | gttaata cctattggca aatctaccaa tggttctaaa | |
| cDNA | g ccgaaaaacc ctgagggaga tcacgcacta | |
| strand | aattaat (SEQ ID NO: 325) | |
| 22227T | gttaata cctattggca aatctaccaa tggttctaaa | |
| cDNA | a ccgaaaaacc ctgagggaga tcacgcacta | |
| strand | aattaat (SEQ ID NO: 326) | |
| 28932C | gctaga a tggctggca atggcggtga tgctgctctt | N protein |
| template | g c tttgctgc tgcttgacag attgaaccag | A220V |
| strand | cttgagagc (SEQ ID NO: 327) | |
| 28932T | gctaga atggctggca atggcggtga tgctgctctt | |
| template | g t tttgctgc tgcttgacag attgaaccag | |
| strand | cttgagagc (SEQ ID NO: 328) | |
| 28932C | gctctca agctggttca atctgtcaag cagcagcaaa | |
| cDNA | g caagagcagc atcaccgcca ttgccagcca | |
| strand | ttctagc (SEQ ID NO: 329) | |
| 28932T | gctctca agctggttca atctgtcaag cagcagcaaa | |
| cDNA | a caagagcagc atcaccgcca ttgccagcca | |
| strand | ttctagc (SEQ ID NO: 330) | |
| 29645G | ctt gtgcagaatg aattctcgta actacatagc acaa | Orf10 |
| template | g tagat gtagttaact ttaatctcac atagcaatct | V30L |
| strand | tt (SEQ ID NO: 331) | |
| 29645T | ctt gtgcagaatg aattctcgta actacatagc acaa | |
| template | t tagat gtagttaact ttaatctcac atagcaatct | |
| strand | tt (SEQ ID NO: 332) | |
| 29645G | aaagatt gctatgtgag attaaagtta actacatcta | |
| cDNA | c ttgtgctatg tagttacgag aattcattct | |
| strand | gcacaag (SEQ ID NO: 333) | |
| 29645T | aaagatt gctatgtgag attaaagtta actacatcta | |
| cDNA | a ttgtgctatg tagttacgag aattcattct gcacaag | |
| strand | (SEQ ID NO: 334) | |
| 1059C | acacctttt gaaattaaat tggcaaagaa atttgaca | Orf1a |
| template | c c ttcaatgggg aatgtccaaa ttttgtattt | T265I |
| strand | ccctta (SEQ ID NO: 335) | |
| 1059T | acacctttt gaaattaaat tggcaaagaa atttgaca | |
| template | t c ttcaatgggg aatgtccaaa ttttgtattt | |
| strand | ccctta (SEQ ID NO: 336) | |
| 1059C | taaggga aatacaaaat ttggacattc cccattgaag | |
| cDNA | g tgtcaaattt ctttgccaat ttaatttcaa | |
| strand | aaggtgt (SEQ ID NO: 337) | |
| 1059T | taaggga aatacaaaat ttggacattc cccattgaag | |
| cDNA | a tgtcaaattt ctttgccaat ttaatttcaa | |
| strand | aaggtgt (SEQ ID NO: 338) | |
| 25563G | ggctt attgttggcg ttgcacttct tgctgttttt | Orf3aQ57H |
| template | ca g agcgctt ccaaaatcat aaccctcaaa | |
| strand | aagagatggc (SEQ ID NO: 339) | |
| 25563T | ggctt attgttggcg ttgcacttct tgctgttttt | |
| template | ca t agcgctt ccaaaatcat aaccctcaaa | |
| strand | aagagatggc (SEQ ID NO: 340) | |
| 25563G | gccatct ctttttgagg gttatgattt tggaagcgct | |
| cDNA | c tgaaaaacag caagaagtgc aacgccaaca | |
| strand | ataagcc (SEQ ID NO: 341) | |
| 25563T | gccatct ctttttgagg gttatgattt tggaagcgct | |
| cDNA | a tgaaaaacag caagaagtgc aacgccaaca | |
| strand | ataagcc (SEQ ID NO: 342) | |
| 21991-21993 | tgaatt tcaattttgt aatgatccat ttttgggtgt | S protein |
| WT | tta ttaccac aaaaacaaca aaagttggat | Y144 del |
| template | ggaaagtga (SEQ ID NO: 343) | |
| strand | | |
| 21991-21993 | gtgaatt tcaattttgt aatgatccat ttttgggtgt | |
| del | ttaccac aaaaacaaca aaagttggat ggaaagtgag | |
| template | t (SEQ ID NO: 344) | |
| strand | | |
| 21991-21993 | tcactt tccatccaac ttttgttgtt tttgtggtaa | |
| WT | taa acacccaaaa atggatcatt acaaaattga | |
| cDNA | aattca (SEQ ID NO: 345) | |
| strand | | |
| 21991-21993 | actcactt tccatccaac ttttgttgtt tttgtggtaa | |
| del | acacccaaaa atggatcatt acaaaattga aattcac | |
| cDNA | (SEQ ID NO: 346) | |
| strand | | |
| 23271C | aagtttc tgcctttcca acaatttggc agagacattg | S protein |
| template | c tgacactac tgatgctgtc cgtgatccac | A570D |
| strand | agacactt (SEQ ID NO: 347) | |
| 23271A | aagtttc tgcctttcca acaatUggc agagacattg | |
| template | a tgacactac tgatgctgtc cgtgatccac | |
| strand | agacactt (SEQ ID NO: 348) | |
| 23271C | aagtgtc tgtggatcac ggacagcatc agtagtgtca | |
| cDNA | gcaatgtctct gccaaattgt tggaaaggca | |
| strand | gaaactt (SEQ ID 140: 349) | |
| 23271A | aagtgtc tgtggatcac ggacagcatc agtagtgtca | |
| cDNA | t caatgtctct gccaaattgt tggaaaggca | |
| strand | gaaactt (SEQ ID NO: 350) | |
| 23709C | tcagttgct tactctaata actctattgc cataccca | S protein |
| template | c a aattttacta ttagtgttac cacagaaatt | T716I |
| strand | ctacca (SEQ ID NO: 351) | |
| 23709T | tcagttgct tactctaata actctattgc cataccca | |
| template | t a aattttacta ttagtgttac cacagaaatt | |
| strand | ctacca (SEQ ID NO: 352) | |
| 23709C | tggtaga atttctgtgg taacactaat agtaaaattt | |
| cDNA | g tgggtatggc aatagagtta ttagagtaag | |
| strand | caactga (SEQ ID 140: 353) | |
| 23709T | tggtaga atttctgtgg taacactaat agtaaaattt | |
| cDNA | a tgggtatggc aatagagtta ttagagtaag | |
| strand | caactga (SEQ ID NO: 354) | |
| 24506T | tt ttggtgcaat ttcaagtgtt ttaaatgata | S protein |
| template | tcctttcacg tcttgacaaa gttgaggctg | S982A |
| strand | aagtgcaaat att (SEQ ID NO: 355) | |
| 24506G | tt ttggtgcaat ttcaagtgtt ttaaatgata | |
| template | tcctt g cacg tcttgacaaa gttgaggctg | |
| strand | aagtgcaaat att (SEQ ID NO: 356) | |
| 24506T | aatattt gcacttcagc ctcaactttg tcaagacgtg | |
| cDNA | a aaggatatca tttaaaacac ttgaaattgc | |
| strand | accaaaa (SEQ ID 140: 357) | |
| 24506G | aatattt gcacttcagc ctcaactttg tcaagacgtg | |
| cDNA | c aaggatatca tttaaaacac ttgaaattgc | |
| strand | accaaaa (SEQ ID NO: 358) | |
| 24914G | acaa aggaattttt atgaaccaca aatcattact aca | S protein |
| template | g acaaca catttgtgtc tggtaactgt gatgttgtaa | D1118H |
| strand | t (SEQ ID NO: 359) | |
| 24914C | acaa aggaattttt atgaaccaca aatcattact aca | |
| template | c acaaca catttgtgtc tggtaactgt | |
| strand | gatgttgtaa t (SEQ ID NO: 360) | |
| 24914GcDNA | attacaa catcacagtt accagacaca aatgtgttgt | |
| strand | c tgtagtaatg atttgtggtt cataaaaatt | |
| | cctttgt (SEQ ID NO: 361) | |
| 24914C | attacaa catcacagtt accagacaca aatgtgttgt | |
| cDNA | g tgtagtaatg atttgtggtt cataaaaatt | |
| strand | cctttgt (SEQ ID NO: 362) | |
| 241C | gtccgtg ttgcagccga tcatcagcac atctaggttt | 5′ UTR |
| template | c gtccgggtg tgaccgaaag gtaagatgga | |
| strand | gagccttg (SEQ ID NO: 363) | |
| 241T | gtccgtg ttgcagccga tcatcagcac atctaggttt | |
| template | t gtccgggtg tgaccgaaag gtaagatgga | |
| strand | gagccttg (SEQ ID NO: 364) | |
| 241C | caaggct ctccatctta cctttcggtc acacccggac | |
| cDNA | g aaacctagat gtgctgatga tcggctgcaa | |
| strand | cacggac (SEQ ID NO: 365) | |
| 24IT | caaggct ctccatctta cctttcggtc acacccggac | |
| cDNA | a aaacctagat gtgctgatga tcggctgcaa | |
| strand | cacggac (SEQ ID NO: 366) | |
| 3037C | g tgagtttaaa ttggcttcac atatgtattg ttcttt | nsp3 |
| template | c tac cctccagatg aggatgaaga agaaggtgat | F924F |
| strand | tgtg (SEQ ID NO: 367) | |
| 3037T | g tgagtttaaa ttggcttcac atatgtattg | |
| template | ttcttt t tac cctccagatg aggatgaaga | |
| strand | agaaggtgat tgtg (SEQ ID NO: 368) | |
| 3037C | cacaatc accttcttct tcatcctcat | |
| cDNA | ctggagggta g aaagaacaat acatatgtga | |
| strand | agccaattta aactcac | |
| | (SEQ ID NO: 369) | |
| 3037T | cacaatc accttcttct tcatcctcat | |
| cDNA | ctggagggta a aaagaacaat acatatgtga | |
| strand | agccaattta aactcac (SEQ ID NO: 370) | |
| 14408C | gcaaacttta atgttttatt ctctacagtg | RdRp |
| template | ttcccac c ta caagttttgg accactagtg | P323L |
| strand | agaaaaatat ttgtt (SEQ ID NO: 371) | |
| 14408T | gcaaacttta atgttttatt ctctacagtg | |
| template | ttcccac t ta caagttttgg accactagtg | |
| strand | agaaaaatat ttgtt | |
| | (SEQ ID NO: 372) | |
| 14408C | aacaaat atttttctca ctagtggtcc | |
| cDNA | aaaacttgta g gtgggaacac tgtagagaat | |
| strand | aaaacattaa agtttgc | |
| | (SEQ ID NO: 373) | |
| 14408T | aacaaat atttttctca ctagtggtcc | |
| cDNA | aaaacttgta a gtgggaacac tgtagagaat | |
| strand | aaaacattaa agtttgc | |
| | (SEQ ID NO: 374) | |
| 261440 | gagc ctgaagaaca tgtccaaatt cacacaatcg | Orf3a |
| template | acg g ttcatc cggagttgtt aatccagtaa | G251V |
| strand | tggaaccaat t (SEQ ID NO: 375) | |
| 261447 | gagc ctgaagaaca tgtccaaatt cacacaatcg | |
| template | acg t ttcatc cggagttgtt aatccagtaa | |
| strand | tggaaccaat t (SEQ ID NO: 376) | |
| 26144G | aattggt tccattactg gattaacaac | |
| cDNA | tccggatgaa c cgtcgattgt gtgaatttgg | |
| strand | acatgttctt caggctc (SEQ ID NO: 377) | |
| 26144T | aattggt tccattactg gattaacaac | |
| cDNA | tccggatgaa a cgtcgattgt gtgaatttgg | |
| strand | acatgttctt caggctc | |
| | (SEQ ID NO: 378) | |
| 29095C | gta ctgccactaa agcatacaat gtaacacaag | N protein |
| template | cttt c ggcag acgtggtcca gaacaaaccc | F274F |
| strand | aaggaaattt tg (SEQ ID NO: 379) | |
| 29095T | gta ctgccactaa agcatacaat gtaacacaag | |
| template | cttt t ggcag acgtggtcca gaacaaaccc | |
| stnind | aaggaaattt tg (SEQ ID NO: 380) | |
| 29095C | caaaatt tccttgggtt tgttctggac | |
| cDNA | cacgtctgcc g aaagcttgtg ttacattgta | |
| strand | tgctttagtg gcagtac (SEQ ID NO: 381) | |
| 29095T | caaaatt tccttgggtt tgttctggac | |
| cDNA | cacgtctgcc a aaagcttgtg ttacattgta | |
| strand | tgctttagtg gcagtac (SEQ ID NO: 382) | |
| 22865G | tta taaattacca gatgatttta caggctgcgt | S protein |
| template | tata g cttgg aattctaaca atcttgattc | A435S |
| strand | taaggttggt gg (SEQ ID NO: 383) | |
| 22865T | tta taaattacca gatgatttta caggctgcgt | |
| template | tata t cttgg aattctaaca atcttgattc | |
| strand | taaggttggt gg (SEQ ID NO: 384) | |
| 22865G | ccaccaa ccttagaatc aagattgtta | |
| cDNA | gaattccaag c tataacgcag cctgtaaaat | |
| strand | catctggtaa tttataa (SEQ ID NO: 385) | |
| 22865T | ccaccaa ccttagaatc aagattgtta | |
| cDNA | gaattccaag a tataacgcag cctgtaaaat | |
| strand | catctggtaa tttataa (SEQ ID NO: 386) | |
| 22320A | ttacttgc tttacataga agttatttga | S protein |
| template | ctcctggtg a ttcttcttca ggttggacag | D253G |
| strand | ctggtgctgc agcttat (SEQ ID NO: 539) | |
| 22320G | ttacttgc tttacataga agttatttga | |
| template | ctcctggtg g ttcttcttca ggttggacag | |
| strand | ctggtgctgc agcttat (SEQ ID NO: 540) | |
| 22320A | ataagct gcagcaccag ctgtccaacc | |
| cDNA | tgaagaagaa t caccaggag tcaaataact | |
| strand | tctatgtaaa gcaagtaa (SEQ ID NO: 541) | |
| 22320G | ataagct gcagcaccag ctgtccaacc tgaagaagaa | |
| cDNA | c caccaggag tcaaataact tctatgtaaa | |
| strand | gcaagtaa (SEQ ID NO: 542) | |

TABLE 24
Blocking Oligonucleotides-S Protein Mutations

| B1ocking | | Protein/ |
|---|---|---|
| O1igo | | Amino |
| Name | Sequence | Acid |
| 21765-21770 | gttac ttggttccat | S protein |
| BO1-1 | gctatacatg | Δ69-70 |
| OLA1 | (SEQ ID NO: 175) | |
| 21765-21770 | ccaatgttac ttggttccat | |
| BO1-2 | gcta | |
| OLA1 | (SEQ ID NO: 176) | |
| 21765-21770 | tctctgggac caatggtact | |
| BO2 | aag (SEQ ID NO: 177) | |
| OLA1 | | |
| 21765-21770 | ccattg gtcccagagacatgta | |
| BO1-1 | (SEQ ID NO: 178) | |
| OLA2 | | |
| 21765-21770 | cttagtaccattg | |
| BO1-2 | gtcccagaga | |
| OLA2 | (SEQ ID NO: 179) | |
| 21765-21770 | tagcatggaaccaagtaac | |
| BO2 | attgg | |
| OLA2 | (SEQ ID NO: 180) | |
| 23063 | a atcatatggt ttccaaccca | S protein |
| BO1 | ct Y | N501Y |
| OLA1 | (SEQ ID NO: 181) | |
| 23063 | atggtgt tggttaccaa | |
| BO2 | ccatac | |
| OLA1 | (SEQ ID NO: 182) | |
| 23063 | tatggttggtaaccaac | |
| BO1 | accat Y | |
| OLA2 | (SEQ ID NO: 183) | |
| 23063 | agtgggttggaaaccata | |
| BO2 | tgat tg | |
| OLA2 | (SEQ ID NO: 184) | |
| 23604 | ctag ttatcagact | S protein |
| BO1 | cagactaatt ctc M | P68IH |
| OLA1 | (SEQ ID NO: 185) | |
| 23604 | tcggcg ggcacgtag | |
| BO2 | (SEQ ID NO: 186) | |
| OLA1 | | |
| 23604 | ctacgtgcccgccga B | |
| BO1 | (SEQ ID NO: 187) | |
| OLA2 | | |
| 23604 | gagaattagtctgagtctgataa | |
| BG2 | ctagc (SEQ ID NO: 188) | |
| OLA2 | | |
| 22132 | ggaaaaca gggtaatttc | S protein |
| BO1 | aaaaatctta gK | R190S |
| OLA1 | (SEQ ID NO: 189) | |
| 22132 | gaatttgt gtttaagaat | |
| BO2 | attgatggtt attttaaaat | |
| OLA1 | at (SEQ ID NO: 190) | |
| 22132 | taaaataaccatcaata | |
| BO1 | ttcttaaac acaaattc | |
| OLA2 | M (SEQ ID NO: 191) | |
| 22132 | ctaagatttttgaaattacc | |
| BO2 | ctgttttcct | |
| OLA2 | (SEQ ID NO: 192) | |
| 22206 | acgccta ttaatttagt | S protein |
| BO1 | gcgtg R | D215G |
| OLA1 | (SEQ ID NO: 193) | |
| 22206 | tctc cctcagggtt | |
| BO2 | tttcgg (SEQ ID NO: 194) | |
| OLA1 | | |
| 22206 | ccgaaaaaccctgagggaga | |
| BO1 | Y (SEQ ID NO: 195) | |
| OLA2 | | |
| 22206 | cacgc actaaatt | |
| BO2 | aataggcgtg | |
| OLA2 | (SEQ ID NO: 196) | |
| 22917 | attc taaggttggt | S protein |
| BO1 | ggtaattata attacc K | L452R |
| OLA1 | (SEQ ID NO: 197) | |
| 22917 | gta tagattgttt | |
| BO2 | aggaagtcta atctcaaac | |
| OLA1 | (SEQ ID NO: 198) | |
| 22917 | gtttgagattagacttccta | |
| BO1 | aacaatcta tac M | |
| OLA2 | (SEQ ID NO: 199) | |
| 22917 | ggtaattataattaccaccaa | |
| BO2 | ccttagaat | |
| OLA2 | (SEQ 1DNO: 200) | |
| 23012 | agcacacctt gtaatggtgt | S protein |
| BO1 | t R | E4S4K |
| OLA1 | (SEQ ID NO: 201) | |
| 23012 | aaggtttt aattgttact | |
| BO2 | ttcctttaca atc | |
| OLA1 | (SEQ ID NO: 202) | |
| 23012 | tgat tgtaaaggaaagtaa | |
| BO1 | caattaaaacctt Y | |
| OLA2 | (SEQ ID NO: 203) | |
| 23012 | aacaccattacaaggtgt | |
| BG2 | gcta | |
| OLA2 | (SEQ ID NO: 204) | |
| 23664 | gcctacact atgtcacttg | S protein |
| BO1 | gtg Y | A701V |
| OLA1 | (SEQ ID NO: 205) | |
| 23664 | agaaaa ttcagttgct | |
| BO2 | tactctaata actct | |
| OLA1 | (SEQ ID NO: 206) | |
| 23664 | agagttattagagtaagc | |
| BO1 | aactgaattttct R | |
| OLA2 | (SEQ ID NO: 207) | |
| 23664 | caccaagtgacatagt | |
| BO2 | gtaggc | |
| OLA2 | (SEQ ID NO: 208) | |
| 22813 | gctccagg gcaaactgga | S protein |
| BO1 | aM K | K417N |
| OLA1 | (SEQ ID NO: 209) | |
| 22813 | attgctg attataatta | |
| BO2 | taaattacca | |
| OLA1 | gatgatttta c | |
| | (SEQ ID NO: 210) | |
| 22813 | gtaaaatcatctggtaatt | |
| BO1 | tataattataatcagcaat | |
| OLA2 | M | |
| | (SEQ ID NO: 211) | |
| 22813 | Kttccagtttgc cctggagc | |
| BO2 | (SEQ ID NO: 212) | |
| OLA2 | | |
| 22812 | gctccagg gcaaactgga | S protein |
| BO1 | a M | K417T |
| OLA1 | (SEQ ID NO: 213) | |
| 22812 | Kattgctg attataatta | |
| BO2 | taaattacca gatgatttta | |
| OLA1 | c | |
| | (SEQ ID NO: 214) | |
| 22812 | atcatctggtaatttataa | |
| BO1 | ttataatcagcaatMK | |
| OLA2 | (SEQ ID NO: 215) | |
| 22812 | ttccagtttgc cctggagc | |
| BO2 | (SEQ ID NO: 216) | |
| OLA2 | | |
| 22227 | tc cctcagggtt | S protein |
| BO1 | tttcgg Y | A222V |
| OLA1 | (SEQ ID NO: 387) | |
| 22227 | ttt agaaccattg | |
| BO2 | gtagatttgc ca | |
| OLA1 | (SEQ ID NO: 388) | |
| 22227 | ggcaaatctaccaat | |
| BO1 | ggttctaaa R | |
| OLA2 | (SEQ ID NO: 389) | |
| 22227 | ccgaaaaaccctga | |
| BO2 | gggaga | |
| OLA2 | (SEQ ID NO: 390) | |
| 28932 | cggtga tgctgctctt | N protein |
| BO1 | g Y | A220V |
| OLA1 | (SEQ ID NO: 391) | |
| 28932 | tttgctgc tgcttgacag | |
| BO2 | attg | |
| OLA1 | (SEQ ID NO: 392) | |
| 28932 | caatctgtcaagcagcag | |
| BO1 | caaaR | |
| OLA2 | (SEQ ID NO: 393) | |
| 28932 | caagagcagcatcaccgc | |
| BO2 | (SEQ ID NO: 394) | |
| OLA2 | | |
| 29645 | tg aattctcgta | Orflo |
| BO1 | actacatagc acaa | V30L |
| OLA1 | K | |
| | (SEQ ID NO: 395) | |
| 29645 | tagat gtagttaact | |
| BO2 | ttaatctcac atagc | |
| OLA1 | (SEQ ID NO: 396) | |
| 29645 | gctatgtgagattaaa | |
| BO1 | gttaactacatcta | |
| OLA2 | M | |
| | (SEQ ID NO: 397) | |
| 29645 | ttgtgctatgtagtta | |
| BO2 | cgagaattca | |
| OLA2 | (SEQ ID NO: 398) | |
| 1059 | tt gaaattaaat | Orfla |
| BO1 | tggcaaagaa atttgaca | T265I |
| OLA1 | Y (SEQ ID NO: 399) | |
| 1059 | c ttcaatgggg | |
| BO2 | aatgtccaaa | |
| OLA1 | ttttg | |
| | (SEQ ID Ng: 400) | |
| 1059 | aaaatttggacattcc | |
| BO1 | ccattgaag R | |
| OLA2 | (SEQ ID NO: 401) | |
| 1059 | tgtcaaatttctttgc | |
| BO2 | caatttaatttcaaa | |
| OLA2 | (SEQ ID NO: 402) | |
| 25563 | g ttgcacttct | Orf3a |
| BO1 | tgctgttttt ca K | Q57H |
| OLA1 | (SEQ ID NO: 403) | |
| 25563 | agcgctt ccaaaatcat | |
| BO2 | aaccct | |
| OLA1 | (SEQ ID NO: 404) | |
| 25563 | gggttatgattttggaa | |
| BO1 | gcgct M | |
| OLA2 | (SEQ ID NO: 405) | |
| 25563 | tgaaaaacagcaa | |
| BO2 | gaagtgcaac | |
| GLA2 | (SEQ ID NO: 406) | |
| 21991- | ttttgt aatgatccat | S protein |
| 21993 | ttttgggtgt tta | Y144 del |
| BO1-1 | (SEQ ID NO: 407) | |
| OLA1 | | |
| 21991-21993 | aattttgt aatgatccat | |
| BO1-2 | ttttgggtgt | |
| OLA1 | (SEQ ID NO: 408) | |
| 21991-21993 | ttaccac aaaaacaaca | |
| BO2 | aaagttggat | |
| OLA1 | (SEQ ID NO: 409) | |
| 21991-21993 | ccaacttttgttgttt | |
| BO1-1 | ttgtggtaataa | |
| OLA2 | (SEQ ID NO: 410) | |
| 21991-21993 | atccaacttttgttgtt | |
| BO1-2OLA2 | tttgtggtaa | |
| | (SEQ ID NO: 411) | |
| 21991-21993 | /5Phos/acacccaaaaatgga | S protein |
| BO2 | tcattacaaaatt/3Bio/ | A570D |
| OLA2 | (SEQ ID NO: 412) | |
| 23271 | cca acaatttggc | |
| BO1 | agagacattg M | |
| OLA1 | (SEQ ID NO: 413) | |
| 23271 | tgacactac tgatgctgtc | |
| BO2 | cg (SEQ ID NO: 414) | |
| OLA1 | | |
| 23271 | cggacagcatca gtagtgtca | |
| BO1 | K (SEQ ID NO: 415) | |
| OLA2 | | |
| 23271 | Caatgtctctgccaaattgttg | |
| BO2 | ga (SEQ ID NO: 416) | |
| OLA2 | | |
| 23709 | tctaata actctattgc | S protein |
| BO1 | cataccca Y | T716I |
| OLA1 | (SEQ ID NO: 417) | |
| 23709 | a aattttacta ttagtgttac | |
| BO2 | cacagaaatt c | |
| OLA1 | (SEQ ID NO: 418) | |
| 23709 | gaatttctgtggtaacactaa | |
| BO1 | tagtaaaattt R | |
| OLA2 | (SEQ ID NO: 419) | |
| 23709 | tgggtatg gcaatagagtt | |
| BG2 | attagagtaa | |
| OLA2 | (SEQ ID NO: 420) | |
| 24506 | gcaat ttcaagtgtt | S protein |
| BO1 | ttaaatgata tcctt | S982A |
| OLA1 | K (SEQ ID NO: 421) | |
| 24506 | cacg tcttgacaaa | |
| BO2 | gttgaggc | |
| OLA1 | (SEQ ID NO: 422) | |
| 24506 | gcctcaactttgtcaag | |
| BO1 | acgtg M | |
| OLA2 | (SEQ ID NO: 423) | |
| 24506 | aaggatatcatttaaaa | |
| BO2 | cacttgaa attgca | |
| OLA2 | (SEQ ID NO;424) | |
| 24914 | gaattttt atgaaccaca | S protein |
| BO1 | aatcattact aca S | D1118H |
| OLA1 | (SEQ ID NO: 425) | |
| 24914 | acaaca catttgtgtc | |
| BO2 | tggtaact | |
| OLA1 | (SEQ ID NO: 426) | |
| 24914 | gttaccagacacaaatgt | |
| BO1 | gttgt S | |
| OLA2 | (SEQ ID NO: 427) | |
| 24914 | tgtagtttatgatt tgtg | |
| BO2 | gttcatttaaaattcc | |
| OLA2 | (SEQ ID NO: 428) | |
| 241 | cga tcatcagcac | 5′ UTR |
| BO1 | atctaggttt Y | |
| OLA1 | (SEQ ID NO: 429) | |
| 241 | gtccgggtg tgaccgaaag | |
| BO2 | (SEQ fD NO: 430) | |
| OLA1 | | |
| 241 | cggtcacacccggac R | |
| BO1 | (SEQ ID NO: 431) | |
| OLA2 | | |
| 241 | aaacctagatgtgctga | |
| BO2 | tgatcg (SEQ ID NO: 432) | |
| OLA2 | | |
| 3037 | a ttggcttcac | nsp3 |
| BO1 | atatgtattg | F924F |
| OLA1 | ttcttt Y | |
| | (SEQ ID NO: 433) | |
| 3037 | tac cctccagatg | |
| BO2 | aggatgaaga | |
| OLA1 | (SEQ ID NO: 434) | |
| 3037 | cttcatcctcatc | |
| BO1 | tggagggta R | |
| OLA2 | (SEQ ID NO: 435) | |
| 3037 | aaagaacaatacatatg | |
| BO2 | tgaagccaatt | |
| OLA2 | (SEQ ID NO: 436) | |
| 14408 | ttttatt ctctacagtg | RdRp |
| BO1 | ttcccac Y | P323L |
| OLA1 | (SEQ ID NO: 437) | |
| 14408 | ta caagttttgg | |
| BO2 | accactagtg aga | |
| OLA1 | (SEQ ID NO: 438) | |
| 14408 | ctcactagtggtccaaa | |
| BO1 | acttgta R | |
| OLA2 | (SEQ ID NO: 439) | |
| 14408 | gtgggaacac tgtagag | |
| BO2 | aataaaact | |
| OLA2 | (SEQ ID NO: 440) | |
| 26144 | gtccaaatt cacacaatcg | Orf3a |
| BO1 | acg K | G251V |
| OLA1 | (SEQ ID NO: 441) | |
| 26144 | ttcatc cggagttgtt | |
| BO2 | aatccag | |
| OLA1 | (SEQ ID NO: 442) | |
| 26144 | ctggattaacaactccg | |
| BO1 | gatgaa M | |
| OLA2 | (SEQ ID NO: 443) | |
| 26144 | cgtcgattgtgtgaat | |
| BO2 | ttggaca | |
| OLA2 | (SEQ ID NO: 444) | |
| 29095 | a agcatacaat | N protein |
| BO1 | gtaacacaag cttt Y | F274F |
| OLA1 | (SEQ ID NO: 445) | |
| 29095 | ggcag acgtggtcca | |
| BO2 | gaa (SEQ ID NO: 446) | |
| OLA1 | | |
| 29095 | ctggaccacgtctgcc | |
| BO1 | R (SEQ ID NO: 447) | |
| OLA2 | | |
| 29095 | aaagcttgtgttacattg | |
| BO2 | tatgcttta | |
| OLA2 | (SEQ ID NO: 448) | |
| 22865 | ca gatgatttta | S protein |
| BO1 | caggctgcgt tata K | A435S |
| OLA1 | (SEQ ID NO: 449) | |
| 22865 | cttgg aattctaaca | |
| BO2 | atcttgattc taagg | |
| OLA1 | (SEQ ID NO: 450) | |
| 22865 | cttagaatcaagattgt | |
| BO1 | tagaattccaag M | |
| OLA2 | (SEQ ID NO: 451) | |
| 22865 | tataacgcagcctgtaa | |
| BO2 | aatcatct | |
| OLA2 | (SEQ ID NO: 452) | |
| 22320 | acataga agttatttga | S protein |
| BO1 | ctcctggtg R | D253G |
| OLA1 | (SEQ ID NO: 543) | |
| 22320 | ttcttcttca | |
| BO2 | ggttggacag ctg | |
| OLA1 | (SEQ ID NO: 544) | |
| 22320 | ag ctgtccaacc | |
| BO1 | tgaagaagaa Y | |
| OLA2 | (SEQ ID NO: 545) | |
| 2232 | caccaggag tcaaataact | |
| BO2 | tctatgtaaa | |
| OLA2 | (SEQ ID NO: 546) | |

TABLE 25
Primers-S Protein Mutations

| | | Protein/ |
|---|---|---|
| | | Amplified |
| | | for |
| Oligo Name | Sequence | AA change |
| S panel long | acgtggt | S protein |
| range F | gtttatt | 69-70del- |
| primer 1 | accctga | A701V |
| (21661-23812) | c | |
| | (SEQ ID | |
| | NO: 217) | |
| S panel long | gctgcat | |
| range R | tcagttg | |
| primer 1 | aatcacc | |
| (21661-23812) | a | |
| | (SEQ ID | |
| | NO: 218) | |
| S panel long | TTCCAAT | S protein |
| range F | GTTACTT | 69-70del- |
| primer 2 | GGTTCCA | A701V |
| (21739-23707) | TGCT | |
| | (SEQ ID | |
| | NO: 219) | |
| S panel long | GGGTATG | |
| range R | GCAATAG | |
| primer 2 | AGTTATT | |
| (21739-23707) | AGAGTAA | |
| | GC | |
| | (SEQ ID | |
| | NO 220) | |
| S panel medium | ACATTCAA | S protein |
| F primer 1 | CTCAGGAC | 69-70del, |
| (21706-22341) | TTGTTCTT | R190S, |
| | ACC | D215G |
| | (SEQ ID | |
| | NO: 221) | |
| S panel medium | GCTGTCC | |
| R primer I | AACCTGA | |
| (21706-22341) | AGAAGAA | |
| | TCAC | |
| | (SEQ ID | |
| | NO: 222) | |
| S panel medium | CAGGAAGA | S protein |
| F primer 2 | GAATCAGC | K417N, |
| (22624-23321) | AACTGTGT | K417T, |
| | T | L452R, |
| | (SEQ ID | E484K, |
| | NO: 223) | N501Y |
| S panel medium | TGTCAAGA | |
| R primer 2 | ATCTCAAG | |
| (22624-23321) | TCTCTGTG | |
| | GAT | |
| | (SEQ ID | |
| | NO: 224) | |
| S panel medium | ATCAACTTA | S protein |
| F primer 3 | CTCCTACTT | D614G, |
| (23442-24103) | GGCGTGTT | P681H, |
| | (SEQ ID | A701V |
| | NO: 225) | |
| S panel medium | TCTAGCAGC | |
| R primer 3 | AATATCACC | |
| (23442-24103) | AAGGCAAT | |
| | (SEQ ID | |
| | NO: 226) | |
| S panel short | CGTGGTGTT | S protein |
| F primer 1 | TATTACCCT | 69-70 del |
| (21662-21918) | GACA | |
| | (SEQ ID | |
| | NO: 453) | |
| S panel short | ATAAGTAG | |
| R primer 1 | GGACTGGG | |
| (21662-21918) | TCTTCG | |
| | (SEQ ID | |
| | NO: 454) | |
| S panel short | GGATGGAAA | S protein |
| F primer 2 | GTGAGTTCA | R190S, D215G |
| (22017-22341) | GAGT | |
| | (SEQ ID | |
| | NO: 455) | |
| S panel short | GCTGTCCAA | |
| R primer 2 | CCTGAAGAA | |
| (22017-22341) | GAAT | |
| | (SEQ ID | |
| | NO 456) | |
| S panel short | CAGGCTGCG | S protein |
| F primer 3 | TTATAGCTT | L452R, |
| (22851-23128) | GGAA | E484K, |
| | (SEQ ID | N501Y |
| | NO: 457) | |
| S panel short | TGCTGGTGC | |
| R primer 3 | ATGTAGAAG | |
| (22851-23128) | TTCA | |
| | (SEQ ID | |
| | NO 458) | |
| S panel short | TGAGTGTGA | S protein |
| F primer 4 | CATACCCAT | P681H, |
| (23542-23813) | TGGT | A701V |
| | (SEQ ID | |
| | NO: 459) | |
| S panel short | TGCTGCATT | |
| R primer 4 | CAGTTGAAT | |
| (23542-23813) | CACC | |
| | (SEQ ID | |
| | NO: 460) | |
| S panel short | ACATTCAAC | S protein |
| F primer 5 | TCAGGACTT | 69-70 del |
| (21706-21873) | GTTCTTACC | |
| | (SEQ ID | |
| | NO: 461) | |
| S panel short | CAGCCTCTT | |
| R primer 5 | ATTATGTTA | |
| (21706-21873) | GACTTCTCA | |
| | GTG | |
| | (SEQ ID | |
| | NO: 462) | |
| S panel short | GTTGGATGG | S protein |
| F primer 6 | AAAGTGAGT | |
| (22014-22252) | TCAGAGTTT | |
| | (SEQ ID | |
| | NO: 463) | |
| S panel short R | TGGCA | R190S, |
| primer 6 | AATCT | D215G |
| (22014-22252) | ACCAA | |
| | TGGTT | |
| | CTAAA | |
| | GC | |
| | (SEQ ID | |
| | NO: 464) | |
| S panel short F | CAGGA | S protein |
| primer 7 | AGAGA | K417N, |
| (22624-22871) | ATCAG | K417T |
| | CAACT | |
| | GTGTT | |
| | (SEQ ID | |
| | NO: 465) | |
| S panel short R | TCCAA | |
| primer 7 | GCTAT | |
| (22624-22871) | AACGC | |
| | AGCCT | |
| | GTAA | |
| | (SEQ ID | |
| | NO: 466) | |
| S panel short F | CAATC | S protein |
| primer 8 | TTGAT | L452R, |
| (22879-23099) | TCTAA | E484K, |
| | GGTTG | N501Y |
| | GTGGT | |
| | (SEQ ID | |
| | NO: 467) | |
| S panel short R | GTACT | |
| primer 8 | ACTAC | |
| (22879-23099) | TCTGT | |
| | ATGGT | |
| | TGGTA | |
| | AC | |
| | (SEQ ID | |
| | NO: 468) | |
| S panel short F | TCCAC | S protein |
| primer 9 | AGACA | D614G |
| (23296-23647) | CTTGA | |
| | GATTC | |
| | TTGAC | |
| | AT | |
| | (SEQ ID | |
| | NO: 469) | |
| S panel short R | AACAC | |
| primer 9 | GCCAA | |
| (23296-23647) | GTAGG | |
| | AGTAA | |
| | GTTGA | |
| | T | |
| | (SEQ ID | |
| | NO: 470) | |
| S panel short F | GCTAG | S protein |
| primer 10 | TTATC | P681H, |
| (23576-23815) | AGACT | A701V |
| | CAGAC | |
| | TAATT | |
| | CTCCT | |
| | C | |
| | (SEQ ID | |
| | NO: 47t) | |
| S panel short R | ATTGC | |
| primer 10 | TGCAT | |
| (23576-23815) | TCAGT | |
| | TGAAT | |
| | CACCA | |
| | C | |
| | (SEQ ID | |
| | NO: 472) | |
| S panel short F | GTCAG | S protein |
| primer 11 | TGTGT | 69-70 del |
| (21600-21871) | TAATC | |
| | TTACA | |
| | ACCAG | |
| | AACTC | |
| | (SEQ ID | |
| | NO: 473) | |
| S panel short R | GCCTC | |
| primer 11 | TTATT | |
| (21600-21871) | ATGTT | |
| | AGACT | |
| | TCTCA | |
| | GTGGA | |
| | (SEQ ID | |
| | NO: 474) | |
| S panel short F | TGGAT | S protein |
| primer 12 | GGAAA | R190S, |
| (22016-22331) | GTGAG | D2I5G |
| | TTCAG | |
| | AGTTT | |
| | (SEQ ID | |
| | NO: 475) | |
| S panel short R | CTGAA | |
| primer 12 | GAAGA | |
| (22016-22331) | ATCAC | |
| | CAGGA | |
| | GTCAA | |
| | (SEQ ID | |
| | NO: 476) | |
| S panel short F | TACAG | S protein |
| primer 13 | GCTGC | L452R, |
| (22849-23134) | GTTAT | E484K, |
| | AGCTT | N50IY |
| | GGAA | |
| | (SEQ ID | |
| | NO: 477) | |
| S panel short R | AACAG | |
| primer 13 | TTGCT | |
| (22849-23134) | GGTGC | |
| | ATGTA | |
| | GAAG | |
| | (SEQ ID | |
| | NO: 478) | |
| S panel short F | TCATG | S protein |
| primer 14 | CAGAT | P681H, |
| (23434-23755) | CAACT | A701V |
| | TACTC | |
| | CTACT | |
| | TGG | |
| | (SEQ ID | |
| | NO: 479) | |
| S panel short R | CATAG | |
| primer 14 | ACACT | |
| (23434-23755) | GGTAG | |
| | AATTT | |
| | CTGTG | |
| | GTAAC | |
| | (SEQ ID | |
| | NO: 480) | |
| S panel short F | aacgc | S protein |
| primer 15 | cacca | K417N, |
| (22589-22860) | gattt | K417T |
| | gcatc | |
| | (SEQ ID | |
| | NO: 481) | |
| S panel short R | acgca | |
| primer 15 | gcctg | |
| (22589-22860) | taaaa | |
| | tcatc | |
| | t(SEQID | |
| | NO: 482) | |
| S panel short F | actgt | S protein |
| primer 16 | gttgc | K417N, |
| (22641-22865) | tgatt | K417T |
| | attct | |
| | gtc | |
| | (SEQ ID | |
| | NO: 483) | |
| S panel short R | ctata | |
| primer 16 | acgca | |
| (22641-22865) | gcctg | |
| | tnaaa | |
| | tca | |
| | (SEQ ID | |
| | NO: 484) | |
| S panel short F | aaact | S protein |
| primer 17 | tgtgc | K417N, |
| (22561 alt start) | ccttt | K417T |
| | tggtg | |
| | (SEQ ID | |
| | NO: 485) | |
| Full S gene | agggg | S Protien |
| F primer 1 | tactg | |
| | ctgtt | |
| | atgtc | |
| | t | |
| | (SEQ ID | |
| | NO: 486) | |
| Full S gene | gcagt | |
| R primer 1 | agcgc | |
| | gaaca | |
| | aaatc | |
| | (SEQ ID | |
| | NO: 487) | |
| Full S gene | atttg | S Protien |
| F primer 2 | acatg | |
| | agtaa | |
| | atttc | |
| | ccctt | |
| | (SEQ ID | |
| | NO: 488) | |
| Full S gene | gcatc | |
| R primer 2 | cttga | |
| | tttca | |
| | ccttg | |
| | c | |
| | (SEQ ID | |
| | NO: 489) | |
| Full S gene | ttaaa | S Protien |
| F primer 3 | ttaag | |
| | gggta | |
| | ctgct | |
| | gttat | |
| | g | |
| | (SEQ ID | |
| | NO: 490) | |
| Full S gene | cgcga | |
| R primer 3 | acaaa | |
| | atctg | |
| | aagga | |
| | gt | |
| | (SEQ ID | |
| | NO: 491) | |

TABLE 26
Targeting and Detection Probes-
Locations 8782, 28144, 23403, and 11083

| Probe Name | Probe sequence |
|---|---|
| S 8782 US | ACTGGTAACCCA |
| Probe OLA1 | GACATGATCGGT |
| | gctgattttgac |
| | acatggtttagt |
| | (SEQ ID NO: 492) |
| L 8782 US | ACTCCCTGTGGG |
| Probe OLA1 | TGAGCTTAATGG |
| | gctgattttgac |
| | acatggtttagc |
| | (SEQ ID NO: 493) |
| 8782 DS | /5phos/cagcgt |
| Probe OLA1 | ggtggtagttatt |
| | atact/3bto/ |
| | (SEQ ID NO: 494) |
| S 28144 US | CATTTGGTCATT |
| Probe OLA1 | GGTTCAAGACGA |
| | gatatcggtaat |
| | tatacagtttcc |
| | tgttc |
| | (SEQ ID NO: 495) |
| L 28144 US | CTGGTCCGTTGT |
| Probe OLA1 | GGTCCTTCTAAC |
| | gatatcggtaat |
| | tatacagtttcc |
| | tgttt |
| | (SEQ ID NO: 496) |
| 28144 DS | /5Phos/acctttt |
| Probe OLA1 | acaattaattgcca |
| | ggaac/3bio/ |
| | (SEQ ID NO: 497) |
| 23403A US | CTAATAGCTCCT |
| Probe OLA1 | GTGCCCTCGTAT |
| | CAGGTTGCTGT |
| | TCTTTATCAGG A |
| | (SEQ ID NO: 498) |
| 23403G US | AATCCGTCGACTA |
| Probe OLA1 | GCCTGAGAATT C |
| | AGGTTGCTGTTCT |
| | TTATCAGG G |
| | (SEQ ID NO: 499) |
| 23403 DS | /5Phos/TGTTAAC |
| Probe OLA1 | TGCACAGAAGTC |
| | CCT/3Bio/ |
| | (SEQ ID NO: 500) |
| 11083G US | GTGGCAACAGAAA |
| Probe OLA1 | TCAGGTGGTGA a |
| | gtactc aatggt |
| | cttt gttctttt |
| | tt ttg |
| | (SEQ ID NO: 501) |
| U083T US | AGAGGACT GCTA |
| Probe OLA1 | AAGGTTT GTAGG |
| | agtaclc aatg |
| | gtcttt gttctt |
| | tttt ttt |
| | (SEQ ID NO: 502) |
| 11083 DS | /5Phos/tatgaaa |
| Probe OLA1 | atgccttttt |
| | accttttgct |
| | a/3Bio/ |
| | (SEQ ID NO: 503) |
| 11083G US | GTGGCAACAGAAAT |
| Probe OLA2 | CAGGTGGTGA gca |
| | aaaggtaaaaaggc |
| | attttcatac |
| | (SEQ ID NO: 504) |
| 11083T US | AGAGGACTGCTAAAG |
| Probe OLA2 | GTTTGTAGG gcaaa |
| | aggtaaaaaggcatt |
| | ttcataa |
| | (SEQ ID NO: 505) |
| 11083 DS | /5Phos/aaaaaaaag |
| Probe OLA2 | aacaaagaccattga |
| | gtactc/3Bio/ |
| | (SEQ ID NO: 506) |

TABLE 27
Synthetic Templates-Locations
8782, 28144, 23403, and 11083

| Oligo Name | Sequence |
|---|---|
| S 8782 | ttgcta |
| Template | acaaacatgc |
| Strand | tgattttgac |
| | acatggttta |
| | gtcagcgtgg |
| | tggtagttat |
| | actaatgaca |
| | aagcttgcc |
| | (SEQ |
| | ID |
| | NO: |
| | 507) |
| L | ttgcta |
| 8782 | acaaacatgc |
| Template | tgattttgac |
| Strand | acatggttta |
| | gccagcgtgg |
| | tggtagttat |
| | actaatgaca |
| | aagcttgcc |
| | (SEQ |
| | ID |
| | NO: |
| | 508) |
| S | GGCAAGCTT |
| 8782 | TGTCATTAGT |
| cDNA | ATAACTACCA |
| Strand | CCACGCTGAC |
| | TAAACCATGT |
| | GTCAAAATCA |
| | GCATGTTTGT |
| | TAGCAA |
| | (SEQ |
| | ID |
| | NO: |
| | 509) |
| L | GGCAAGCTT |
| 8782 | TGTCATTAGT |
| cDNA | ATAACTACCA |
| Strand | CCACGCTGGC |
| | TAAACCATGT |
| | GTCAAAATCA |
| | GCATGTTTGT |
| | TAGCAA |
| | (SEQ |
| | ID |
| | NO: |
| | 510) |
| S | cagt |
| 28144 | acatcgatat |
| Template | cggtaattat |
| Strand | acagtttcct |
| | gttcaccttt |
| | tacaattaat |
| | tgccaggaac |
| | ctaaattggg |
| | t |
| | (SEQ |
| | ID |
| | NO: |
| | 511) |
| L | cagt |
| 28144 | acatcgatat |
| Template | cggtaattat |
| Strand | acagtttcct |
| | gtttaccttt |
| | tacaattaat |
| | tgccaggaac |
| | ctaaattggg |
| | t |
| | (SEQ |
| | ID |
| | NO: |
| | 512) |
| S | A |
| 28144 | CCCAAT |
| cDNA | TTAG |
| Strand | GTTCCTGGCA |
| | ATTAATTGTA |
| | AAAGGTGAAC |
| | AGGAAACTGT |
| | ATAATTACCG |
| | ATATCGATGT |
| | ACTG |
| | (SEQ |
| | ID |
| | NO: |
| | 513) |
| L | A |
| 28144 | CCCAATTTAG |
| cDNA | GTTCCTGGCA |
| Strand | ATTAATTGTA |
| | AAAGGTAAAC |
| | AGGAAACTGT |
| | ATAATTACCG |
| | ATATCGATGT |
| | ACTG |
| | (SEQ |
| | ID |
| | NO: |
| | 514) |
| 23403A | acaaa |
| Template | tacttctaac |
| Strand | caggttgctg |
| | ttctttatca |
| | gg a |
| | tgttaac |
| | tgcacagaag |
| | tccctgttgc |
| | tattcatgca |
| | (SEQ |
| | ID |
| | NO: |
| | 515) |
| 23403G | acaaa |
| Template | tacttctaac |
| Strand | caggttgctg |
| | ttctttatca |
| | gg g |
| | tgttaac |
| | tgcacagaag |
| | tccctgttgc |
| | tattcatgca |
| | (SEQ |
| | ID |
| | NO: |
| | 516) |
| 23403A | TGCATGAATA |
| cDNA | GCAACAGGGA |
| Strand | CTTCTGTGCA |
| | GTT |
| | AAC |
| | A |
| | T |
| | CC |
| | TGATAAAGAA |
| | CAGCAACCTG |
| | GTTAGAAGTA |
| | TTTGT |
| | (SEQ |
| | ID |
| | NO: |
| | 517) |
| 23403G | TGCATGAATA |
| cDNA | GCAACAGGGA |
| Strand | CTTCTGTGCA |
| | GTTAACACCC |
| | TGATAAAGAA |
| | CAGCAACCTG |
| | GTTAGAAGTA |
| | TTTGT |
| | (SEQ |
| | ID |
| | NO: |
| | 518) |
| 11083G | tactc |
| Template | cagagtactc |
| Strand | aatggtcttt |
| | gttctttttt |
| | tt g |
| | tatgaaa |
| | atgccttttt |
| | accttttgct |
| | atgggtatta |
| | (SEQ |
| | ID |
| | NO: |
| | 5I9) |
| 11083T | tactc |
| Template | cagagtactc |
| Strand | aatggtcttt |
| | gttctttttt |
| | tt t |
| | tatgaaa |
| | atgccttttt |
| | accttttgct |
| | atgggtatta |
| | (SEQ |
| | ID |
| | NO: |
| | 520) |
| 11083G | taatacccata |
| cDNA | gcaaaaggtaa |
| Strand | aaaggcatttt |
| | cata c |
| | aaaaaaaagaa |
| | caaagaccatt |
| | gagtactctgg |
| | acta |
| | (SEQ |
| | ID |
| | NO: |
| | 521) |
| 11083T | taatacccata |
| cDNA | gcaaaaggtaa |
| Strand | aaaggcatttt |
| | cata c |
| | aaaaaaaagaa |
| | caaagaccatt |
| | gagtactctgg |
| | acta |
| | (SEQ |
| | ID |
| | NO: |
| | 522) |

TABLE 28
Blocking Oligonucleotides-Locations
8782, 28144, 23403, and 11083

| Blocking | |
|---|---|
| Oligo Name | Sequence |
| 8782 US | gctgattttgaca |
| OLA1 BO | catggtttagY |
| | (SEQ ID NO: 523) |
| 8782 DS | cagcgtggtggt |
| OLA1 BO | agttatact |
| | (SEQ ID NO: 524) |
| 28144 US | gatatcggtaatta |
| OLA1 BO | tacagtttcctgttY |
| | (SEQ ID NO: 525) |
| 28144 DS | acettUacaatta |
| OLA1 BO | attgccaggaac |
| | (SEQ ID NO: 526) |
| 23403 US | CAGGTTGCTGTTC |
| OLA1 BO | TTTATCAGG R |
| | (SEQ ID NO: 527) |
| 23403 DS | TGTTAACTGCAC |
| OLA1 BO | AGAAGTCCCT |
| | (SEQ ID NO: 528) |
| 11083 US | agtactc aatggtcttt |
| OLA1 BO | gttctttttt tt K |
| | (SEQ ID NO: 529) |
| 11083 DS | tatgaaa atgccttttt |
| OLA1 BO | accttttgct a |
| | (SEQ ID NO: 530) |
| 11083 US | gcaaaaggtaaaaag |
| OLA2 BO | gcattttcata M |
| | (SEQ ID NO: 531) |
| 11083 DS | aaaaaaaagaacaaa |
| OLA2 BO | gaccattgagtactc |
| | (SEQ ID NO: 532) |

### Example 29. Assessment of Brain Injury Biomarkers in COVID-19 Patients

A novel ultrasensitive multiplexed assay panel measuring GFAP, total tau, and NF-L was developed, with higher assay sensitivity for tau than any currently available tau assay (e.g., from Quanterix). This is the first time that such assays have been multiplexed. Plasma samples were obtained from COVID-19 patients in an intensive care unit (ICU), hospitalized and not requiring intensive care (non-ICU), outpatients testing positive for COVID-19, and outpatients testing negative for COVID-19. The plasma samples were tested with the novel assay panel measuring levels of GFAP, total Tau, and NF-L. Results of the measured initial biomarker levels at the first hospital visit are shown in FIG. 53A. Results of the measured biomarker levels at 1, 2, 3, 4, and 5 weeks after the first hospital visit are shown in FIG. 53B.

GFAP and NF-L showed progressively higher median blood concentrations as severity of illness increased from outpatient treatment to hospitalization to intensive care. Median concentration of tau in blood was higher among COVID-19 patients hospitalized in an ICU compared to hospitalized COVID-19 patients not requiring intensive care or outpatients. COVID-19 patients treated as outpatients for mild to moderate disease had normal blood levels of GFAP, NF-L, and tau comparable to non-hospitalized controls without COVID-19. Among COVID-19 patients in the ICU, NF-L blood levels rose over 2 to 3 weeks and persisted for up to five weeks. Blood levels of GFAP and tau both peaked at approximately one week and steadily declined over the subsequent four weeks.

### Example 30. Assessment of Immune Checkpoint Biomarkers COVID-19 Patients

EDTA-treated plasma samples were obtained from subjects who were uninfected with COVID-19, subjects who had asymptomatic COVID-19 infection as determined by a positive PCR test, and subjects who were hospitalized from a COVID-19 infection. The hospitalized subjects were further classified as those having moderate disease: those having severe disease that required admission into the intensive care unit (ICU): or those having severe disease that required ICU admission who subsequently passed away. The plasma samples were obtained from the subjects with moderate disease at the time of hospital admission, and from subjects with severe disease at the time of ICU admission.

The plasma samples were tested on two multiplexed immunoassay panels for immune checkpoint biomarkers. Plasma samples were diluted 50-fold were used for an assay panel measuring CD78, CD28, CD40L, CTLA-4, GITR, LAG3, OX40, PD1, and TIGIT. Plasma samples were diluted 100-fold were used for an assay panel measuring Tie-2, gp130, and TIM-3.

Results of the measured biomarker levels are shown in FIG. 54. As shown in the results, CD27, CTLA-4, LAG3, OX40, PD1, TIGIT and TIM-3 were observed to be elevated in the subjects with severe disease relative to subjects with moderate disease or asymptomatic subjects.

### Example 31. Host Biomarker Assays in COVID-19 Patients

In order to identify biomarkers that may indicate active or recent SARS-CoV-2 infection, concentrations of various cytokines, acute phase reactants, and other biomarkers of inflammation and tissue damage were measured in nasal swabs, saliva, and serum obtained from patients with active or recent infection with SARS-CoV-2 as well as healthy controls without current or prior infection with SARS-CoV-2. The following biomarkers were measured: IFN-γ, IL-1β, IL-2, IL-4, IL-6, IL-8, IL-10, IL-12p70, IL-13, TNF-α, IL-1α, IL-5, IL-7. IL-12p40. IL-15, IL-16, IL-17A, GM-CSF, VEGF-A, TNFβ, IL-17A/F, IL-17-B, IL-17-C, IL-17-D, IL-1RA, IL-3, IL-9, TSLP, MCP-1, MCP-4, Eotaxin-1, Eotaxin-3, MIP1a, MIP1b, IP-10, MDC, TARC, bFGF, VEGF-A, VEGF-C, VEGF-D, sFlt-1, Tie-2, PIGF, E-selectin, P-selectin, Thrombomodulin, ICAM-3, lectin binding protein (LBP), CRP, SAA, ICAM-1, VCAM-1, MIP-3α, IFN-α, IFN-β, TAC, procalcitonin (PCT), Granzyme B, IFN-2α, CD20, CD5, and Flt3-L.

A general increase of cytokines, acute phase reactants, and other biomarkers of inflammation and tissue damage in nasal swabs and saliva was observed among patients with active infection with SARS-CoV-2 compared to healthy controls. In saliva, infected individuals had especially high levels for IL-1β, IL-10, IL-8, IL-6, IL-13, GM-CSF, IL-15, IL-17A, MDC, MCP-1, Flt-1, PIGF, VEGF-C, ICAM-3, Thrombomodulin, CRP, LBP, ICAM-1, SAA, FLT3L, and IL-2. In nasal swabs, infected individuals had especially high levels for IFN-γ, IL-7, IL-15, MCP-1, Flt-1, IFN-2α, IL-15. Notably, increases in these biomarkers were more prominent in saliva and nasal swabs than observed in serum. Thus, these biomarkers individually or in combination can be used to identify individuals with active infection with SARS-CoV-2 or other viruses based on testing of nasal swabs, saliva, and/or serum.

### Example 32. Clinical Characterization of Serology Assay Panels and Formats

Clinical characterization of assay panels and formats as shown below was carried out using 200 pre-2019 COVID-19-negative serum samples and 200 PCR-confirmed COVID-19-positive serum samples. The samples were grouped from time of serum collection relative to positive PCR test (0 to 14, 15 to 28, 29 to 56, or 57+ days post-diagnosis (Dx).

Assay Panels and Formats:

(1) Indirect (Classical) IgG Serology and ACE2 Competition:

Antigens in Panel A: SARS-CoV-2 N protein, S protein, and S-RBD;

Antigens in Panel B: Wild type (WT) SARS-CoV-2 N protein: S protein and S-RBD from WT SARS-CoV-2 and SARS-CoV-2 variants B.1.1.7. B.1.351, and P.1;

(2) Bridging Serology:

Antigens in Panel C: SARS-CoV-2 N protein and S-RBD.

Summary of Results for WT Antigens:

The WT antigens provided excellent clinical performance, even for early positive samples. The measured specificities for the different antigens/formats ranged from 98.5% to 100%, measured sensitivities for late positives (15+ days from Dx) ranged from 93.8% to 98.3%, measured sensitivities for early positives (≤14 days from Dx) ranged from 65.8% to 92.1%. Assay signals for the Bridging Serology assay format increased with time across the time groups, indicating the Bridging Serology assay format may measure affinity maturation of antibody responses over time.

All antigens and assay formats provided excellent separation of the negative and positive samples. While sensitivity for detecting early (≤14 day) infection was good, for most assay panels and formats there was a significant increase in concentration and sensitivity for samples collected >14 days after diagnosis. Interestingly, while antibody concentrations measured by the Indirect Serology and ACE2 Competition assay formats generally plateaued for time points longer than 14 days, concentrations measured by the Bridging Serology assay format showed significant increases with time across the measured time range. Since the Bridging Serology assay format requires two antibody-antigen binding interactions, it is generally biased towards measuring only high affinity antibodies and may be more sensitive in measuring changes in average antibody affinities as antibody responses mature over time.

The ROC curves showed near ideal separation of negative and late positive samples, as demonstrated by AUC values ranging from 0.983 to 0.992 (an AUC of 1 indicates perfect separation).

All assay formats and antigens showed excellent clinical performance. Point estimates for sensitivity for convalescent samples (15+ days post diagnosis) ranged from 93.8% to 98.3%, with the highest sensitivities obtained by the Indirect IgG Serology and ACE2 Competition assay formats using SARS-CoV-2 S protein as the antigen. Sensitivity during acute infection (0 to 14 days post diagnosis) was lower, which was expected since the antibody response is still developing during this period, but still quite good with point estimates ranging from 65.8% to 92.1%. The Indirect IgG Serology and ACE2 Competition assay formats with SARS-CoV-2 S protein as the antigen were also the most sensitive for detecting acute infection. The assays were very specific with point estimates for specificity ranging from 98.5% to 100%. The performance of all the assays was well within the requirements stated in the United States Food and Drug Administration (FDA) guidance or use of COVID-19 serology tests under an Emergency Use Authorization (EUA).

Summary of Results for SARS-CoV-2 Variants/Mutant Antigens:

Panels that include antigens from SARS-CoV-2 variant lineages can provide important tools for assessing the immunity of individuals to the different variants. Assays with the S-RBD antigen from the B.1.351 and P.1 variants, which contain the K417N/T or E484K mutations were performed, and the antibody response from the COVID-19-positive samples against the mutant S-RBD were lower as compared to the antibody response against S-RBD without the K417N/T or E484K mutations (i.e., wild type or B.1.1.7 variant). The difference in antibody responses to the wild type vs. mutant S-RBD was much larger when measured by the ACE2 Competition assay relative to the Indirect IgG Serology assay format. These results suggest that individuals infected with a wild type or wild type-like variant may have reduced ability to avoid infection with the B.1.351 or P.1 variants.

Antibody binding to antigens from the four variants was elevated in COVID-positive vs. COVID-negative subjects, although levels were generally lower for the variant antigens, in particular the S-RBD from the B.1.351 and P.1 variants. The difference may stem from the fact that the COVID-19 positive samples were collected in late 2020 and early 2021 in the US when the wild-type lineage was predominant and there was little evidence in the US of the mutations found in the variants. While the number of mutations in the variant antigens is small, at least some of the mutations are likely present in epitopes that are sufficiently immune-dominant to account for a measurable proportion of the antibody response.

1. A kit for detecting multiple antibody biomarkers of interest in a sample, the kit comprising, in one or more vials, containers, or compartments:
a. one or more surfaces comprising multiple binding domains, wherein each of:
an N protein from SARS-CoV-2, an S protein SARS-CoV-2, an S-RBD from SARS-CoV-2, an S-NTD from SARS-CoV-2, an S protein from SARS-CoV, an S protein from MERS-CoV, an S protein from HCoV-HKU1, an S protein from HCoV-OC43, an HA protein from FluB/Brisbane, an HA protein from FluB/Phuket, an HA protein from FluA H1/Michigan, an HA protein from FluA H3/Hong Kong, and an HA protein from FluA H7/Shanghai strain
is immobilized on a distinct binding domain on the one or more surfaces;
b. one or more detection reagents, wherein the detection reagent is an antibody, a viral antigen, or a detectable competitor.

2. A method for detecting multiple antibody biomarkers of interest in a sample, the method comprising:
a. contacting the biological sample with a surface comprising multiple binding domains, wherein each of:
an N protein from SARS-CoV-2, an S protein SARS-CoV-2, an S-RBD from SARS-CoV-2, an S-NTD from SARS-CoV-2, an S protein from SARS-CoV, an S protein from MERS-CoV, an S protein from HCoV-HKU1, an S protein from HCoV-OC43, an HA protein from FluB/Brisbane, an HA protein from FluB/Phuket, an HA protein from FluA H1/Michigan, an HA protein from FluA H3/Hong Kong, and an HA protein from FluA H7/Shanghai strain
is immobilized on a distinct binding domain on the surface;
b. forming a binding complex in each binding domain, wherein the binding complex comprises the viral antigen and an antibody biomarker that binds to the viral antigen;
c. contacting the binding complex in each binding domain with a detection reagent, wherein the detection reagent is an antibody, a viral antigen, or a detectable competitor; and
d. measuring concentration of the antibody biomarker in each binding complex.

3. A kit for detecting a viral infection immune response in a sample, the kit comprising, in one or more vials, containers, or compartments:
a. at least a first binding reagent for a viral antigen and at least a second binding reagent for a biomarker, wherein the first and second binding reagents are immobilized or capable of being immobilized on distinct binding domains on a surface, wherein the viral antigen is a SARS-CoV-2 N protein, a SARS-CoV-2 S protein, or a combination thereof, and wherein the biomarker is GM-CSF, Granzyme A, Granzyme B, IFN-α2a, IFN-β, IFN-γ, IL-10, IL-1RA, IL-4, IL-5, IL-6, IL-7, IL-8, IL-9, IL-12p70, IP-10, I-TAC, MCP-1, MCP-2, MCP-4, MDC, MIP-1α, MIP-1β, TNF-α, VEGF-A, or a combination thereof, and
b. at least a first detection reagent for the viral antigen and at least a second detection reagent for the biomarker.

4. A multiplexed method for determining a viral infection immune response in a sample, the method comprising:
quantifying the amounts of at least one viral antigen and at least one biomarker in the biological sample,
wherein the at least one viral antigen is a SARS-CoV-2 N protein, a SARS-CoV-2 S protein, or a combination thereof, and wherein the at least one biomarker is GM-CSF, Granzyme A, Granzyme B, IFN-α2a, IFN-β, IFN-γ, IL-1β, IL-1RA, IL-4, IL-5, IL-6, IL-7, IL-8, IL-9, IL-12p70, IP-10, I-TAC, MCP-1, MCP-2, MCP-4, MDC, MIP-1α, MIP-1β, TNF-α, VEGF-A, or a combination thereof,
wherein the quantifying comprises:
a. contacting the biological sample with a surface comprising at least a first binding domain and a second binding domain, wherein the first binding domain comprises a binding reagent for the at least one viral antigen, and the second binding domain comprises a binding reagent for the at least one biomarker, thereby forming a binding complex in each binding domain; and
b. detecting the binding complex in each binding domain.

5. A kit for detecting an EV from a SARS-CoV-2 infected cell in a sample, the kit comprising, in one or more vials, containers, or compartments:
a. a first binding reagent that is immobilized or capable of being immobilized on a surface; and
b. a second binding reagent that comprises a nucleic acid probe;
wherein one of the first or second binding reagents binds to an EV surface marker, and the other of the first or second binding reagents binds to a SARS-CoV-2 viral antigen.

6. A method for detecting an EV from a SARS-CoV-2 infected cell in a sample, the method comprising:
a. contacting the sample with (i) a first binding reagent that is immobilized or capable of being immobilized on a surface, and (ii) a second binding reagent that comprises a nucleic acid probe,
wherein one of the first or second binding reagents binds to an EV surface marker, and the other of the first or second binding reagents binds to a SARS-CoV-2 viral antigen, thereby forming a binding complex on the surface comprising the EV and the first and second binding reagents;
b. extending the nucleic acid probe to form an extended oligonucleotide; and
c. measuring the amount of the extended oligonucleotide, thereby detecting the EV from the SARS-CoV-2 infected cell.

7. A kit for detecting an intact SARS-CoV-2 virus a sample, the kit comprising, in one or more vials, containers, or compartments:
a. a binding reagent that specifically binds a first viral antigen on a viral surface, wherein the binding reagent is immobilized or capable of being immobilized on distinct binding domains on a surface;
b. a first detection reagent that binds to a second viral antigen on the viral surface, wherein the first detection reagent comprises a first nucleic acid probe; and
c. a second detection reagent that binds to a third viral antigen on the viral surface, wherein the second detection reagent comprises a second nucleic acid probe.

8. A method for detecting an intact SARS-CoV-2 virus in a sample, the method comprising:
a. contacting the biological sample with (i) a binding reagent that specifically binds a first viral antigen on a viral surface, wherein the binding reagent is immobilized or capable of being immobilized on distinct binding domains on a surface; (ii) a first detection reagent that binds to a second viral antigen on the viral surface, wherein the first detection reagent comprises a first nucleic acid probe; and (iii) a second detection reagent that binds to a third viral antigen on the viral surface, wherein the second detection reagent comprises a second nucleic acid probe, thereby forming a binding complex on the surface comprising the intact SARS-CoV-2 virus, the binding reagent, and the first and second detection reagents;
b. using an extension process that requires the first and second nucleic acid probes to be in proximity, extending the second nucleic acid probe to form an extended oligonucleotide; and
c. measuring the amount of extended oligonucleotide, thereby detecting the intact SARS-CoV-2 virus in the biological sample.

9. A kit for detecting a SARS-CoV-2 nucleic acid in a sample, the kit comprising, in one or more vials, containers, or compartments:
a. an oligonucleotide binding reagent that comprises (i) a targeting agent complement (TAC); (ii) an amplification primer; (iii) a hybridization region comprising a complementary sequence to the SARS-CoV-2 nucleic acid; and (iv) an amplification blocker; and
b. a site-specific nuclease that forms a complex with the oligonucleotide binding reagent.

10. A method for detecting a SARS-CoV-2 nucleic acid in a sample, comprising:
a. contacting the sample with an oligonucleotide binding reagent, wherein the oligonucleotide binding reagent comprises: (i) a targeting agent complement (TAC); (ii) an amplification primer; (iii) a hybridization region comprising a complementary sequence to the SARS-CoV-2 nucleic acid; and (iv) an amplification blocker;
b. forming a binding complex comprising the SARS-CoV-2 nucleic acid and the oligonucleotide binding reagent;
c. contacting the binding complex with a site-specific nuclease that cleaves the oligonucleotide binding reagent to remove the amplification blocker therefrom, thereby generating a first cleaved oligonucleotide comprising the targeting agent complement and the amplification primer, wherein the first cleaved oligonucleotide is not bound to the SARS-CoV-2 nucleic acid;
d. immobilizing the first cleaved oligonucleotide to a detection surface comprising a targeting agent, wherein the targeting agent is a binding partner of the targeting agent complement;
e. extending the first cleaved oligonucleotide on the detection surface to form an extended oligonucleotide; and
f. detecting the extended oligonucleotide, thereby detecting the SARS-CoV-2 nucleic acid in the sample.

11. A kit for detecting a SARS-CoV-2 nucleic acid in a sample, the kit comprising, in one or more vials, containers, or compartments:
a. an oligonucleotide detection reagent that comprises (i) a targeting agent complement (TAC); (ii) an amplification primer; and (iii) an amplification blocker; and
b. a site-specific nuclease having collateral activity.

12. A method for detecting a SARS-CoV-2 nucleic acid in a sample, comprising:
a. contacting the sample with a site-specific nuclease comprising collateral cleavage activity and an oligonucleotide detection reagent, wherein the oligonucleotide detection reagent comprises: (i) a targeting agent complement (TAC); (ii) an amplification primer; and (iii) an amplification blocker;
wherein the site-specific nuclease binds to the SARS-CoV-2 nucleic acid and collaterally cleaves the oligonucleotide detection reagent to remove the amplification blocker therefrom, thereby generating a first cleaved oligonucleotide comprising the targeting agent complement and the amplification primer;
b. immobilizing the first cleaved oligonucleotide to a detection surface comprising a targeting agent, wherein the targeting agent is a binding partner of the targeting agent complement;
c. extending the first cleaved oligonucleotide to form an extended oligonucleotide; and
d. detecting the extended oligonucleotide, thereby detecting the SARS-CoV-2 nucleic acid in the sample.

13. A kit for detecting a SARS-CoV-2 nucleic acid in a sample, the kit comprising, in one or more vials, containers, or compartments:
a. a targeting probe that is complementary to a first region of the SARS-CoV-2 nucleic acid in the sample, wherein the targeting probe comprises an oligonucleotide tag, and wherein the oligonucleotide tag is complementary to a binding reagent immobilized on a surface;
b. a detection probe that is complementary to a second region that is adjacent to the first region of the SARS-CoV-2 nucleic acid, wherein the detection probe comprises a detectable label.

14. A method for detecting a SARS-CoV-2 nucleic acid in a sample, comprising:
a. contacting the sample with: (i) a targeting probe that is complementary to a first region of the SARS-CoV-2 nucleic acid in the sample, wherein the targeting probe comprises an oligonucleotide tag; and (ii) a detection probe that is complementary to a second region that is adjacent to the first region of the SARS-CoV-2 nucleic acid, wherein the detection probe comprises a detectable label;
b. hybridizing the targeting and detection probes to the SARS-CoV-2 nucleic acid;
c. ligating the targeting and detection probes that hybridize with perfect complementarity to the first and second regions of the SARS-CoV-2 nucleic acid to form a ligated target complement comprising the oligonucleotide tag and the detectable label;
d. contacting the product of (c) with a surface comprising a binding reagent immobilized in one or more binding domains, wherein the binding reagent comprises an oligonucleotide complementary to the oligonucleotide tag;
e. forming a binding complex on the surface comprising the binding reagent and the ligated target complement; and
f. detecting the binding complex, thereby detecting the SARS-CoV-2 nucleic acid.

15. The kit of claim 1, wherein the sample comprises a respiratory sample.

16. The kit claim 1, wherein the sample comprises wastewater.

17. The kit of claim 1, wherein the sample is obtained from two or more individuals.

18. The method of claim 2, wherein the sample comprises a respiratory sample.

19. The method of claim 2, wherein the sample comprises wastewater.

20. The method of claim 2, wherein the sample is obtained from two or more individuals.
